(19) United States
(12) Patent Application Publication
(10) Pub. No.: US 2026/0193248 A1
(43) Pub. Date: Jul. 9, 2026

(54) CDK INHIBITORS AND METHODS OF USE THEREOF

(71) Applicant: Relay Therapeutics, Inc., Cambridge, MA (US)

(72) Inventors: Jing He, Cambridge, MA (US); Nicolas A. Pabon, Cambridge, MA (US); Lucian V. Dipietro, Cambridge, MA (US); Kevin David Raynor, Cambridge, MA (US); Jessica Pitch, Cambridge, MA (US); Thomas H. McLean, Cambridge, MA (US); Alexander M. Taylor, Cambridge, MA (US); Yuanchi Zhao, Cambridge, MA (US); Erich W. Baum, Cambridge, MA (US); Heike Schoenherr, Cambridge, MA (US); Paul Robert Fleming, Cambridge, MA (US)

(21) Appl. No.: 19/132,427

(22) PCT Filed: Dec. 1, 2023
(86) PCT No.: PCT/US2023/082156

§ 371 Date: May 23, 2025

Related U.S. Application Data
(60) Provisional application No. 63/385,905, filed on Dec. 2, 2022.
(60) Provisional application No. 63/578,835, filed on Aug. 25, 2023.
(60) Provisional application No. 63/590,346, filed on Oct. 13, 2023.

(51) Int. Cl. C07D 417/14; A61K 31/437; A61K 31/4375; A61K 31/4433; A61K 31/4436; A61K 31/4439; A61K 31/444; A61K 31/4545; A61K 31/4709; A61K 31/4725; A61K 31/496; A61K 31/4985; A61K 31/501; A61K 31/505; A61K 31/506; A61K 31/513; A61K 31/517; A61K 31/519; A61K 31/52; A61K 31/53; A61K 31/5377; A61K 31/538; A61K 31/5386; A61K 31/55; A61K 31/551; A61K 31/553; A61K 31/675; C07B 59/00; C07D 239/42; C07D 401/04; C07D 401/14; C07D 403/04; C07D 403/14; C07D 405/04; C07D 405/12; C07D 405/14; C07D 409/14; C07D 411/14; C07D 413/14; C07D 471/04; C07D 471/10; C07D 471/14; C07D 473/34; C07D 487/04; C07D 487/08; C07D 487/10; C07D 487/20; C07D 491/048; C07D 491/107; C07D 491/20; C07D 493/04; C07D 493/10; C07D 495/04; C07D 498/08; C07D 498/14; C07D 513/04; C07D 513/14; C07D 519/00; C07F 9/6558; C07F 9/6568

(52) U.S. Cl. CPC C07D 417/14; A61K 31/437; A61K 31/4375; A61K 31/4433; A61K 31/4436; A61K 31/4439; A61K 31/444; A61K 31/4545; A61K 31/4709; A61K 31/4725; A61K 31/496; A61K 31/4985; A61K 31/501; A61K 31/505; A61K 31/506; A61K 31/513; A61K 31/517; A61K 31/519; A61K 31/52; A61K 31/53; A61K 31/5377; A61K 31/538; A61K 31/5386; A61K 31/55; A61K 31/551; A61K 31/553; A61K 31/675; C07B 59/002; C07D 239/42; C07D 401/04; C07D 401/14; C07D 403/04; C07D 403/14; C07D 405/04; C07D 405/12; C07D 405/14; C07D 409/14; C07D 411/14; C07D 413/14; C07D 471/04; C07D 471/10; C07D 471/14; C07D 473/34; C07D 487/04; C07D 487/08; C07D 487/10; C07D 487/20; C07D 491/048; C07D 491/107; C07D 491/20; C07D 493/04; C07D 493/10; C07D 495/04; C07D 498/08; C07D 498/14; C07D 513/04; C07D 513/14; C07D 519/00; C07F 9/65586; C07F 9/65685

(57) ABSTRACT

The present disclosure relates to novel compounds and pharmaceutical compositions thereof, and methods for inhibiting the activity of CDK enzymes with the compounds and compositions of the disclosure. The present disclosure further relates to, but is not limited to, methods for treating disorders associated with CDK signaling with the compounds and compositions of the disclosure.

### CROSS-REFERENCE TO RELATED APPLICATIONS

This application claims the benefit of priority to U.S. Provisional Appl. No. 63/385,905, filed Dec. 2, 2022, U.S. Provisional Appl. No. 63/578,835, filed Aug. 25, 2023, and U.S. Provisional Appl. No. 63/590,346, filed Oct. 13, 2023, the entirety of each of which is hereby incorporated by reference.

### BACKGROUND

Mammalian cyclin-dependent kinases (CDKs) are a family of serine/threonine kinases that are regulated by direct binding to specific cyclin proteins. CDKs are mainly divided into two groups based on their cellular function: cell cycle regulators (CDK1, CDK2, CDK4, and CDK6) and gene transcription regulators (CDK7, CDK8, CDK9, CDK11, CDK12, and CDK13). Other CDKs (CDK3, CDK5, CDK10, CDK14, CDK16, CDK18, and CDK20) were discovered more recently but their biological function is poorly understood (Malumbres, M. et al. Nat Rev Cancer 2009; Chou J. et al. Cancer Discov 2020).

The role of CDKs has been well-established as the gatekeepers at each phase of cell cycle progression. Cell division post quiescence is initiated by mitogenic stimuli that signal through the MAPK pathway to upregulate Cyclin D, which binds and activates CDK4/6. The activated CDK4/6-Cyclin D complex directly mono-phosphorylates and partially inhibits retinoblastoma protein (RB), releasing E2F transcription factors to start the gene expression program necessary for DNA replication. Cyclin E is one of the E2F target genes, which forms a complex and activates CDK2 to further hyper-phosphorylate RB and fully release E2Fs. The activity of CDK2-Cyclin A complex mediates the final stages of DNA replication while the CDK1-Cyclin B and CDK1-Cyclin A complexes regulate the mitotic stage involving sister chromosome separation into two daughter cells (Malumbres, M. et al. Nat Rev Cancer 2009; Fassl A. et al. Science 2022).

Significant genetic evidence suggests CDK4/6-Cyclin D may be involved in cancer development and progression. In fact, the Cyclin D (CCND1) gene is frequently amplified and overexpressed in a variety of tumor types, including breast cancer (Sanchez-Vega F. et al. Cell 2018). The role of CDK4/6-Cyclin D in breast tumorigenesis was confirmed using in vivo genetic models showing Cyclin D overexpression was sufficient to induce mammary tumors in mice (Wang T C et al. Nature 1994). Conversely, genetic ablation of either Cyclin D or CDK4 abrogated mammary tumor development in mice driven by well-established oncogenes such as RAS and HER2 (Yu Q. et al. Nature 2001; Yu Q. et al. Cancer Cell 2006).

Intriguingly, a subset of breast cancers marked by luminal gene expression and hormone receptor positivity (HR+) were found to be exclusively dependent on CDK4-Cyclin D in preclinical studies (Finn R S, et al. Brest Cancer Res 2009).

### SUMMARY

In some embodiments, the present disclosure encompasses the recognition that there is a need for CDK-selective inhibitor compounds, e.g., CDK4-selective inhibitor compounds, and methods for treating cancers and other disorders with these compounds.

In some embodiments, the present disclosure provides a compound of formula I′:

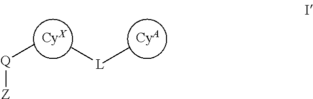

or a pharmaceutically acceptable salt thereof, wherein each of Cy^{A}, Cy^{X}, L, Q, and Z is as defined in embodiments and classes and subclasses herein.

In some embodiments, the present disclosure provides a compound of formula I:

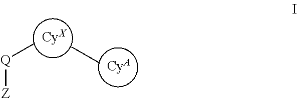

or a pharmaceutically acceptable salt thereof, wherein each of Cy^{A}, Cy^{X}, Q, and Z is as defined in embodiments and classes and subclasses herein.

In some embodiments, the present disclosure provides a compound of formula I-A or a pharmaceutically acceptable salt thereof.

In some embodiments, the present disclosure provides a compound of formula II, III, and IV; or a pharmaceutically acceptable salt thereof.

In some embodiments, the present disclosure provides a compound of formula II-A, II-B, II-C, II-D, II-E, II-F, II-G, II-H, II-I, II-J, II-K, II-L, II-M, II-N, II-O, II-P, II-Q, II-R, II-S, II-T, II-U, II-V, II-W, II-X, II-Y, II-Z, II-AA, II-BB, II-CC, II-DD, II-EE, II-FF, II-GG, II-HH, II-II, II-JJ, II-KK, II-LL, II-MM, II-NN, II-OO, II-PP, II-QQ, II-RR, II-SS, II-TT, II-UU, and II-VV; or a pharmaceutically acceptable salt thereof.

In some embodiments, the present disclosure provides a compound of formula II-A1, II-B1, II-C1, II-D1, II-E1, II-F1, II-G1, II-H1, II-I1, II-J1, II-K1, II-L1, II-M1, II-N1, II-O1, II-P1, and II-Q1; or a pharmaceutically acceptable salt thereof.

In some embodiments, the present disclosure provides a compound of formula II-A2, II-B2, II-C2, II-D2, II-E2, II-F2, II-G2, and II-H2; or a pharmaceutically acceptable salt thereof.

In some embodiments, the present disclosure provides a compound of formula V, VI, VII, VIII, IX, X, XI, XII, XIII, XIV, XV, XVI, XVII, XVIII, XIX, XX, XXI, and XXII; or a pharmaceutically acceptable salt thereof.

In some embodiments, the present disclosure provides a compound of formula XXIII, XXIV, XXV, XXVI, and XXVII; or a pharmaceutically acceptable salt thereof.

In some embodiments, the present disclosure provides a compound of formula XXVIII, XXIX, XXX, XXXI, XXXII, XXXIII, XXXIV, XXXV, XXXVI, XXXVII, XXXVIII, and XXXIX; or a pharmaceutically acceptable salt thereof.

In some embodiments, the present disclosure provides a compound of formula XL, XLI, XLII, and XLIII; or a pharmaceutically acceptable salt thereof.

In some embodiments, the present disclosure provides a compound of formula XLIV, XLV, XLVI, XLVII, XLVIII, XLIX, L, LI, LII, LIII, LIV, and LV; or a pharmaceutically acceptable salt thereof.

In some embodiments, the present disclosure provides a compound of formula LVI, LVII, LVIII, and LIX; or a pharmaceutically acceptable salt thereof.

In some embodiments, the present disclosure provides a compound of formula LX, LXI, LXII, LXIII, LXIV, LXV, LXVI, LXVII, LXVIII, LXIX, LXX, and LXXI; or a pharmaceutically acceptable salt thereof.

In some embodiments, the present disclosure provides a compound of formula LXXII, LXXIII, LXXIV, and LXXV; or a pharmaceutically acceptable salt thereof.

In some embodiments, the present disclosure provides a compound of formula LXXVI, LXXVII, LXXVIII, LXXIX, LXXX, LXXXI, LXXXII, LXXXIII, LXXXIV, LXXXV, LXXXVI, and LXXXVII; or a pharmaceutically acceptable salt thereof.

In some embodiments, the present disclosure provides a compound of formula LXXXVIII, LXXXIX, XC, and XCI; or a pharmaceutically acceptable salt thereof.

In some embodiments, the present disclosure provides a pharmaceutical composition comprising a compound of formula I′, or a pharmaceutically acceptable salt thereof, and a pharmaceutically acceptable carrier, adjuvant, or diluent. In some embodiments, the present disclosure provides a pharmaceutical composition comprising a compound of formula I, or a pharmaceutically acceptable salt thereof, and a pharmaceutically acceptable carrier, adjuvant, or diluent. In some embodiments, the present disclosure provides a pharmaceutical composition comprising a compound of the disclosure, for example, a compound of formula I-A, II, III, IV, II-A, II-B, II-C, II-D, II-E, II-F, II-G, II-H, II-I, II-J, II-K, II-L, II-M, II-N, II-O, II-P, II-Q, II-R, II-S, II-T, II-U, II-V, II-W, II-X, II-Y, II-Z, II-AA, II-BB, II-CC, II-DD, II-EE, II-FF, II-GG, II-HH, II-II, II-JJ, II-KK, II-LL, II-MM, II-NN, II-OO, II-PP, II-QQ, II-RR, II-SS, II-TT, II-UU, II-VV, II-A1, II-B1, II-C1, II-D1, II-E1, II-F1, II-G1, II-H1, II-I1, II-J1, II-K1, II-L1, II-M1, II-N1, II-O1, II-P1, II-Q1, II-A2, II-B2, II-C2, II-D2, II-E2, II-F2, II-G2, II-H2, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX XL, XLI, XLII, XLIII XLIV, XLV, XLVI, XLVII, XLVIII, XLIX, L, LI, LII, LIII, LIV, LV, LVI, LVII, LVIII, LIX, LX, LXI, LXII, LXIII, LXIV, LXV, LXVI, LXVII, LXVIII, LXIX, LXX, LXXI, LXXII, LXXIII, LXXIV, LXXV LXXVI, LXXVII, LXXVIII, LXXIX, LXXX, LXXXI, LXXXII, LXXXIII, LXXXIV, LXXXV, LXXXVI, LXXXVII, LXXXVIII, LXXXIX, XC, or XCI, or a pharmaceutically acceptable salt thereof, and a pharmaceutically acceptable carrier, adjuvant, or diluent.

In some embodiments, the present disclosure provides a method of treating a CDK4-mediated disorder comprising administering to a patient in need thereof a compound of formula I′, or composition comprising said compound. In some embodiments, the present disclosure provides a method of treating a CDK4-mediated disorder comprising administering to a patient in need thereof a compound of formula I, or composition comprising said compound. In some embodiments, the present disclosure provides a method of treating a CDK4-mediated disorder comprising administering to a patient in need thereof a compound of the disclosure, for example, a compound of formula I-A, II, III, IV, II-A, II-B, II-C, II-D, II-E, II-F, II-G, II-H, II-I, II-J, II-K, II-L, II-M, II-N, II-O, II-P, II-Q, II-R, II-S, II-T, II-U, II-V, II-W, II-X, II-Y, II-Z, II-AA, II-BB, II-CC, II-DD, II-EE, II-FF, II-GG, II-HH, II-II, II-JJ, II-KK, II-LL, II-MM, II-NN, II-OO, II-PP, II-QQ, II-RR, II-SS, II-T T, II-UU, II-VV, II-A1, II-B1, II-C1, II-D1, II-E1, II-F1, II-G1, II-H1, II-I1, II-J1, II-K1, II-L1, II-M1, II-N1, II-O1, II-P1, II-Q1, II-A2, II-B2, II-C2, II-D2, II-E2, II-F2, II-G2, II-H2, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX XL, XLI, XLII, XLIII XLIV, XLV, XLVI, XLVII, XLVIII, XLIX, L, LI, LII, LIII, LIV, LV, LVI, LVII, LVIII, LIX, LX, LXI, LXII, LXIII, LXIV, LXV, LXVI, LXVII, LXVIII, LXIX, LXX, LXXI, LXXII, LXXIII, LXXIV, LXXV LXXVI, LXXVII, LXXVIII, LXXIX, LXXX, LXXXI, LXXXII, LXXXIII, LXXXIV, LXXXV, LXXXVI, LXXXVII, LXXXVIII, LXXXIX, XC, or XCI, or a composition comprising said compound. In some embodiments, the present disclosure provides a method of treating a CDK4-mediated disorder comprising administering to a patient in need thereof a compound of the disclosure, for example, a compound of formula I-A, or composition comprising said compound.

In some embodiments, the present disclosure provides a process for providing a compound of formula I′, or synthetic intermediates thereof. In some embodiments, the present disclosure provides a process for providing a compound of formula I, or synthetic intermediates thereof. In some embodiments, the present disclosure provides a process for providing a compound of the disclosure, for example, a compound of formula I-A, II, III, IV, II-A, II-B, II-C, II-D, II-E, II-F, II-G, II-H, II-I, II-J, II-K, II-L, II-M, II-N, II-O, II-P, II-Q, II-R, II-S, II-T, II-U, II-V, II-W, II-X, II-Y, II-Z, II-AA, II-BB, II-CC, II-DD, II-EE, II-FF, II-GG, II-HH, II-II, II-JJ, II-KK, II-LL, II-MM, II-NN, II-OO, II-PP, II-QQ, II-RR, II-SS, II-TT, II-UU, II-VV, II-A1, II-B1, II-C1, II-D1, II-E1, II-F1, II-G1, II-H1, II-I1, II-J1, II-K1, II-L1, II-M1, II-N1, II-O1, II-P1, II-Q1, II-A2, II-B2, II-C2, II-D2, II-E2, II-F2, II-G2, II-H2, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX XL, XLI, XLII, XLIII XLIV, XLV, XLVI, XLVII, XLVIII, XLIX, L, LI, LII, LIII, LIV, LV, LVI, LVII, LVIII, LIX, LX, LXI, LXII, LXIII, LXIV, LXV, LXVI, LXVII, LXVIII, LXIX, LXX, LXXI, LXXII, LXXIII, LXXIV, LXXV LXXVI, LXXVII, LXXVIII, LXXIX, LXXX, LXXXI, LXXXII, LXXXIII, LXXXIV, LXXXV, LXXXVI, LXXXVII, LXXXVIII, LXXXIX, XC, or XCI, or synthetic intermediates thereof.

In some embodiments, the present disclosure provides a process for providing pharmaceutical compositions comprising compounds of formula I′. In some embodiments, the present disclosure provides a process for providing pharmaceutical compositions comprising compounds of formula I. In some embodiments, the present disclosure provides a process for providing pharmaceutical compositions comprising compounds of the disclosure, for example, a compound of formula I-A, II, III, IV, II-A, II-B, II-C, II-D, II-E, II-F, II-G, II-H, II-I, II-J, II-K, II-L, II-M, II-N, II-O, II-P, II-Q, II-R, II-S, II-T, II-U, II-V, II-W, II-X, II-Y, II-Z, II-AA, II-BB, II-CC, II-DD, II-EE, II-FF, II-GG, II-HH, II-IT, II-JJ, II-KK, II-LL, II-MM, II-NN, II-OO, II-PP, II-QQ, II-RR, II-SS, II-TT, II-UU, II-VV, II-A1, II-B1, II-C1, II-D1, II-E1, II-F1, II-G1, II-H1, II-I1, II-J1, II-K1, II-L1, II-M1, II-N1, II-O1, II-P1, II-Q1, II-A2, II-B2, II-C2, II-D2, II-E2, II-F2, II-G2, II-H2, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX XL, XLI, XLII, XLIII XLIV, XLV, XLVI, XLVII, XLVIII, XLIX, L, LI, LII, LIII, LIV, LV, LVI, LVII, LVIII, LIX, LX, LXI, LXII, LXIII, LXIV, LXV, LXVI, LXVII, LXVIII, LXIX, LXX, LXXI, LXXII, LXXIII, LXXIV, LXXV LXXVI, LXXVII, LXXVIII, LXXIX, LXXX, LXXXI, LXXXII, LXXXIII, LXXXIV, LXXXV, LXXXVI, LXXXVII, LXXXVIII, LXXXIX, XC, or XCI.

### DETAILED DESCRIPTION

### 1. General Description of Certain Embodiments

Compounds provided herein, and pharmaceutical compositions thereof, are useful as inhibitors of CDK4. In some embodiments, the present disclosure provides a compound of formula I′:

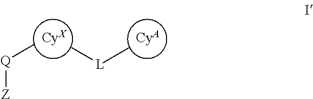

or a pharmaceutically acceptable salt thereof, wherein:
Q is L¹;
Z is H, halogen, or Cy^{B};
L is a covalent bond or a bivalent C₁₋₃ aliphatic group, wherein one carbon is optionally replaced by —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, —S(O)₂—, C₃₋₆ cycloalkylene, or 3-6 membered heterocycloalkylene having 1-2 heteroatoms independently selected from nitrogen, oxygen, and sulfur; wherein each of said C₃₋₆ cycloalkylene and 3-6 membered heterocycloalkylene is optionally substituted with one instance of R¹ or C₁₋₆ aliphatic;
Cy^{A} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{A} is substituted with m instances of R^{A};
Cy^{B} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{B} is substituted with n instances of R^{B};
Cy^{X} is phenyl, naphthyl, a 5-6 membered monocyclic heteroaryl ring having 1-4 heteroatoms independently selected from nitrogen, oxygen, phosphorus, and sulfur; an 8-12 membered bicyclic heteroaryl ring having 1-4 heteroatoms independently selected from nitrogen, oxygen, phosphorus, and sulfur; a 7-12 membered saturated, partially unsaturated, or aromatic bicyclic heterocyclic ring having 1-4 heteroatoms independently selected from nitrogen, oxygen, phosphorus, and sulfur; or a 10-15 membered saturated, partially unsaturated, or aromatic tricyclic heterocyclic ring having 1-6 heteroatoms independently selected from nitrogen, oxygen, phosphorus, and sulfur;
wherein Cy^{X} is substituted with u instances of L²-X⁰;
X⁰ is hydrogen, a halogen, oxo, CN, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{X};
Y is N or CH;
each instance of R^{A}, R^{B}, and R^{X} is independently R¹ or R², wherein R^{A} is substituted by q^{A} instances of R³, R^{B} is substituted by q^{B} instances of R³, and R^{X} is substituted with q^{X} instances of R³; or
   two instances of R^{A}, two instances of R^{B}, two instances of R^{X}, two instances of X⁰, an instance of X⁰ and an instance of R^{A}, or an instance of R^{A} and an instance of R^{X} are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₆ saturated or unsaturated, straight or branched hydrocarbon chain wherein one or two methylene units of the chain are optionally and independently replaced by —CH(R′)—, —C(R′)₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R or —P(O)R₂;
each instance of R² is independently C₁₋₇ aliphatic; —O—C₁₋₇ aliphatic; C₁₋₄ haloalkyl; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{X}, r, t, and u is independently 0, 1, 2, 3, or 4.

Compounds provided herein, and pharmaceutical compositions thereof, are useful as inhibitors of CDK4. In some embodiments, the present disclosure provides a compound of formula I:

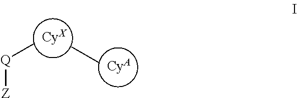

or a pharmaceutically acceptable salt thereof, wherein:
Q is L¹;
Z is H or Cy^{B};
Cy^{A} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{A} is substituted with m instances of R^{A};
Cy^{B} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{B} is substituted with n instances of R^{B};
Cy^{X} is

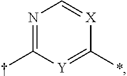

wherein [symbol] represents a bond to Q, and [symbol] represents a bond to Cy^{A};
X is N or CL²-X⁰;
X⁰ is hydrogen, a halogen, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{X};
Y is N or CH;
each instance of R^{A}, R^{B}, and R^{X} is independently R¹ or R², wherein R^{A} is substituted by q^{A} instances of R³, R^{B} is substituted by q^{B} instances of R³, and R^{X} is substituted with q^{X} instances of R³; or
   two instances of R^{A}, two instances of R^{B}, two instances of R^{X}, or an instance of R^{A} and an instance of R^{X} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; —O—C₁₋₇ aliphatic; C₁₋₄ haloalkyl; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{X}, r, and t is independently 0, 1, 2, 3, or 4.

### 2. Compounds and Definitions

Compounds described herein include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference. Chemical names, common names, and chemical structures may be used interchangeably to describe the same structure. If a chemical compound is referred to using both a chemical structure and a chemical name, and an ambiguity exists between the structure and the name, the structure predominates.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. A carbocycle can be, under certain circumstances, a bridged bicyclic or a fused ring such as, e.g., an ortho-fused carbocycle, a spirofused carbocycle, etc. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety, refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of compounds described herein, “aryl” refers to an aromatic ring system which includes, but is not limited to, phenyl, naphthyl, anthracyl and the like, which may bear one or more substituents. It will be appreciated that an “aryl” group can comprise carbon and heteroatom ring members.

The terms “heteroaryl” or “heteroaromatic”, unless otherwise defined, as used herein refers to a monocyclic aromatic 5-6 membered ring containing one or more heteroatoms, for example one to four heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

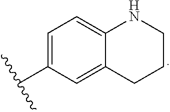 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings (e.g., an ortho-fused bicyclic or a spirofused bicyclic ring), and whose ring system includes one to four heteroatoms, such as nitrogen, oxygen, phosphorus, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

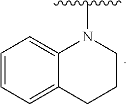 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

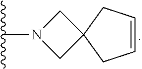 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

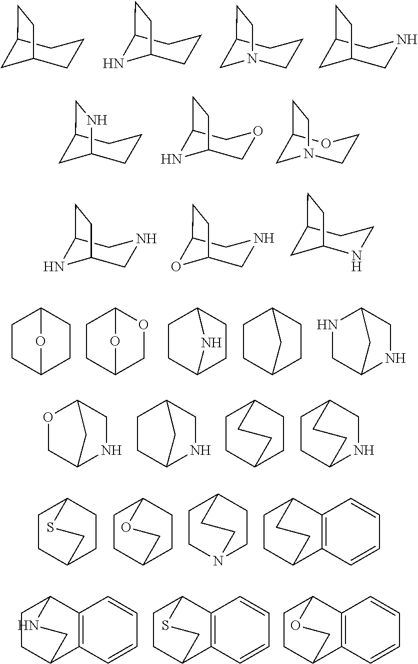

As described herein, compounds described herein may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂, —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{◯}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂₀R^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}. —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds described herein are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds described herein are within the scope of the disclosure. Such compounds are useful, for example, as analytical tools, as probes in biological assays, or as therapeutic agents in accordance with the present disclosure.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J. Pharmaceutics 1986, 33, 201-217; (each hereby incorporated by reference in its entirety).

Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient, when administered to a subject or population suffering from or susceptible to a disease, disorder, and/or condition in accordance with a therapeutic dosing regimen, to treat (e.g., effect beneficial or desired results, including clinical results) the disease, disorder, and/or condition. As such, the effective amount may be sufficient, e.g., to reduce or ameliorate the severity and/or duration of afflictions related to CDK4 signaling, or one or more symptoms thereof, prevent the advancement of conditions or symptoms related to afflictions related to CDK4 signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to CDK4 signaling activity or that may otherwise be relieved by the compounds and/or compositions of the disclosure.

### 3. Description of Exemplary Embodiments

In some embodiments, the present disclosure provides a compound of formula I:

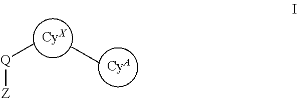

or a pharmaceutically acceptable salt thereof, wherein:
Q is L;
Z is H or Cy^{B};
Cy^{A} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{A} is substituted with m instances of R^{A};
Cy^{B} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{B} is substituted with n instances of R^{B}.
Cy^{X} is

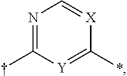

wherein [symbol] represents a bond to Q, and [symbol] represents a bond to Cy^{A};
X is N or CL²-X⁰;
X⁰ is hydrogen, a halogen, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{X};
Y is N or CH;
each instance of R^{A}, R^{B}, and R^{X} is independently R¹ or R², wherein R^{A} is substituted by q^{A} instances of R³, R^{B} is substituted by q^{B} instances of R³, and R^{X} is substituted with q^{X} instances of R³; or
   two instances of R^{A}, two instances of R^{B}, two instances of R^{X}, or an instance of R^{A} and an instance of R^{X} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; —O—C₁₋₇ aliphatic; C₁₋₄ haloalkyl; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{X}, r, and t is independently 0, 1, 2, 3, or 4.

In some embodiments, the present disclosure provides a compound of formula I-A:

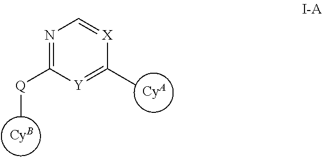

or a pharmaceutically acceptable salt thereof, wherein each of Cy^{A}, Cy^{B}, Q, X, and Y is as defined in embodiments and classes and subclasses herein.

In some embodiments, the present disclosure provides a compound of formula II, III, and IV:

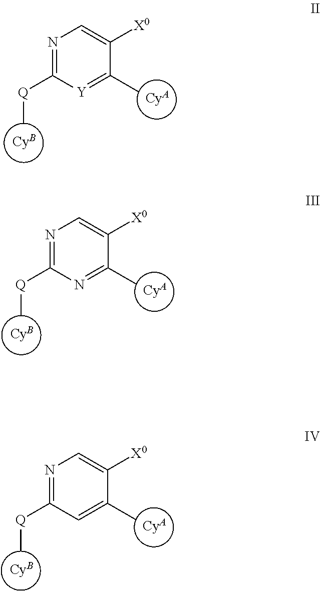

or a pharmaceutically acceptable salt thereof, wherein each of Cy^{A}, Cy^{B}, Q, X⁰, and Y is as defined in embodiments and classes and subclasses herein.

In some embodiments, the present disclosure provides a compound of formula II-A, II-B, II-C, II-D, II-E, II-F, II-G, II-H, II-I, II-J, II-K, II-L, II-M, II-N, II-O, II-P, II-Q, II-R, II-S, II-T, II-U, II-V, II-W, II-X, II-Y, II-Z, II-AA, II-BB, II-CC, II-DD, II-EE, II-FF, II-GG, II-HH, II-II, II-JJ, II-KK, II-LL, II-MM, II-NN, II-OO, II-PP, II-QQ, II-RR, II-SS, II-TT, II-UU, and II-VV:

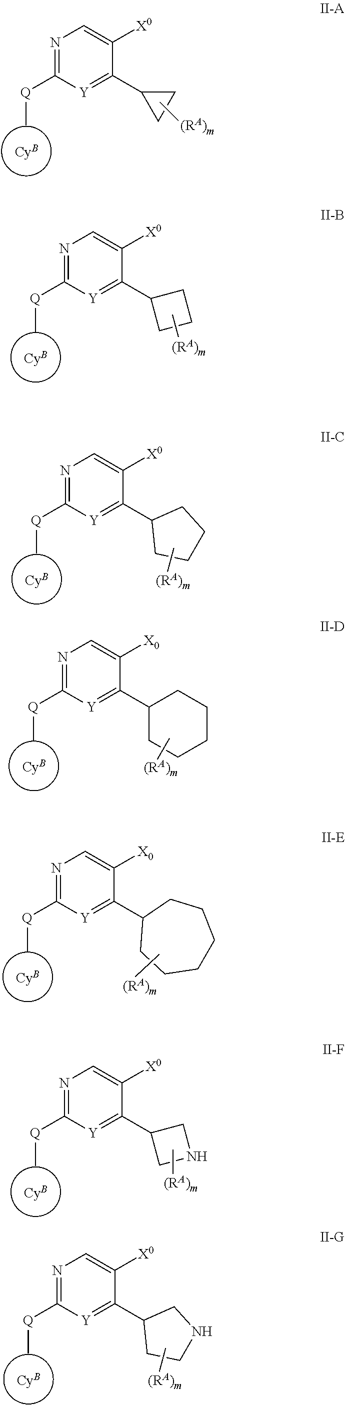

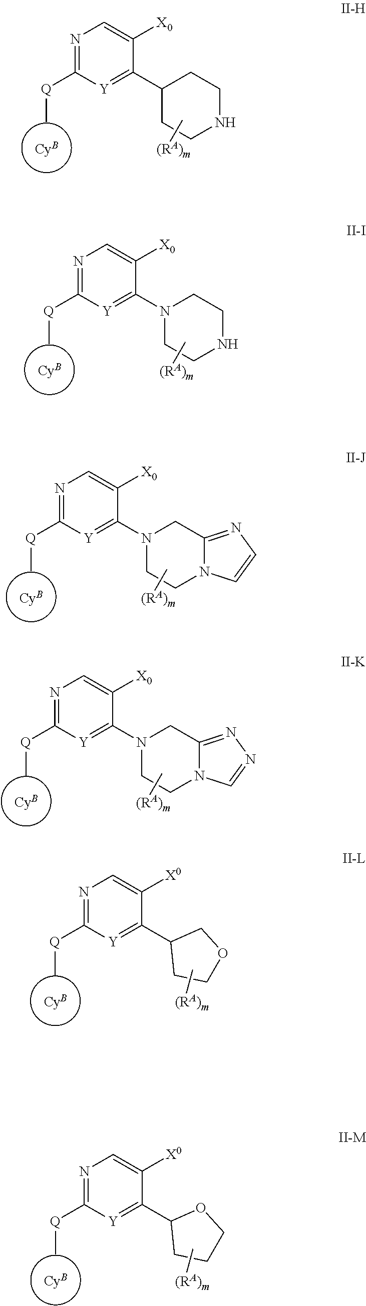

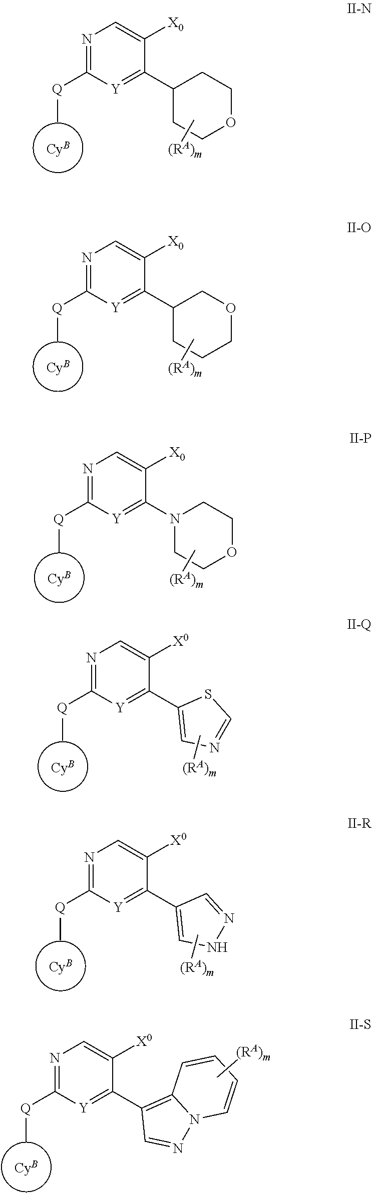

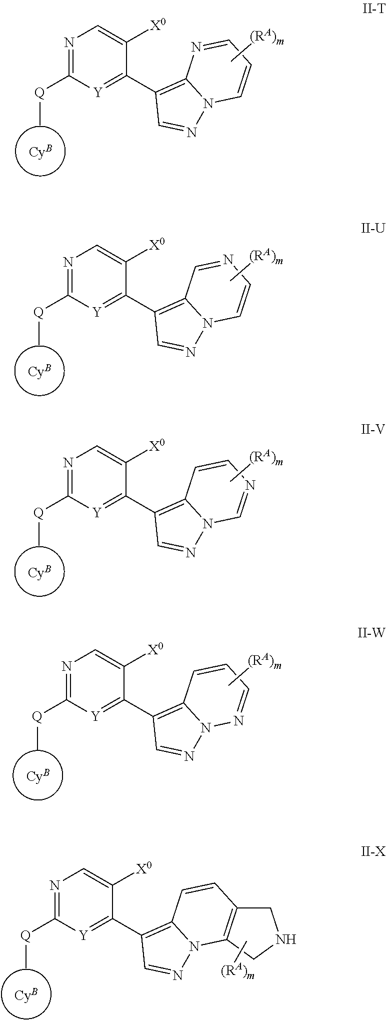

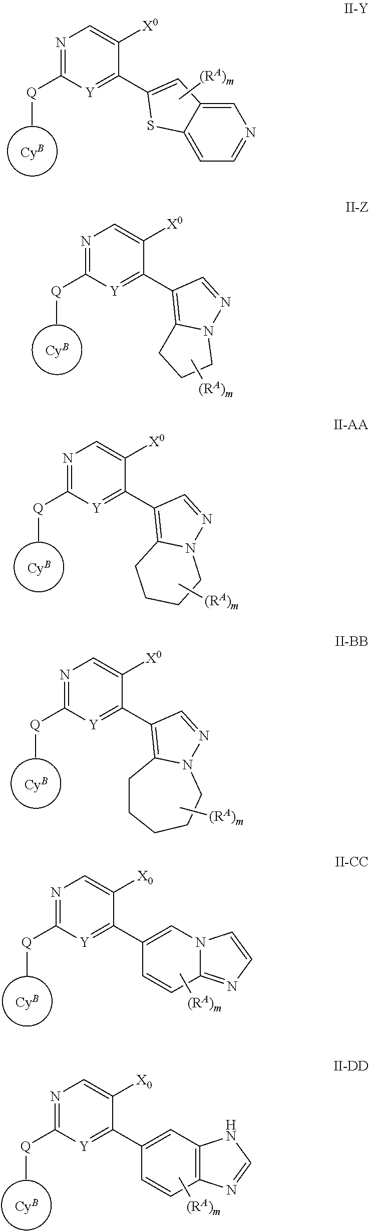

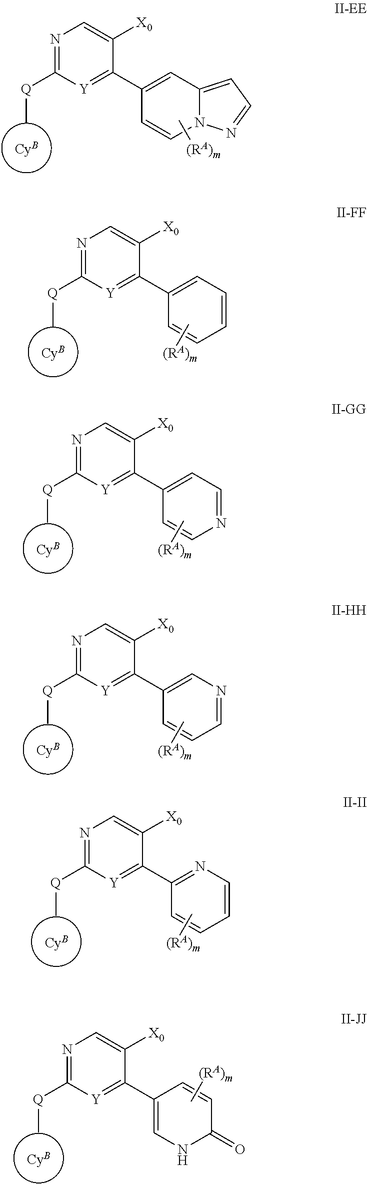

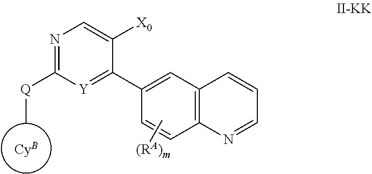

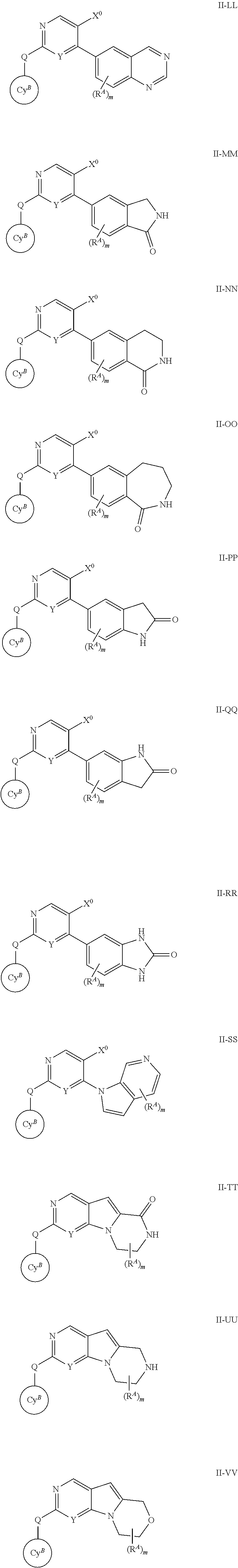

or a pharmaceutically acceptable salt thereof, wherein each of Cy^{B}, Q, X⁰, Y, R^{A} and m is as defined in embodiments and classes and subclasses herein.

In some embodiments, the present disclosure provides a compound of formula II-A1, II-B1, II-C1, II-D1, II-E1, II-F1, II-G1, II-H1, II-I1, II-J1, II-K1, II-L1, II-M1, II-N1, II-O1, II-P1, and II-Q1:

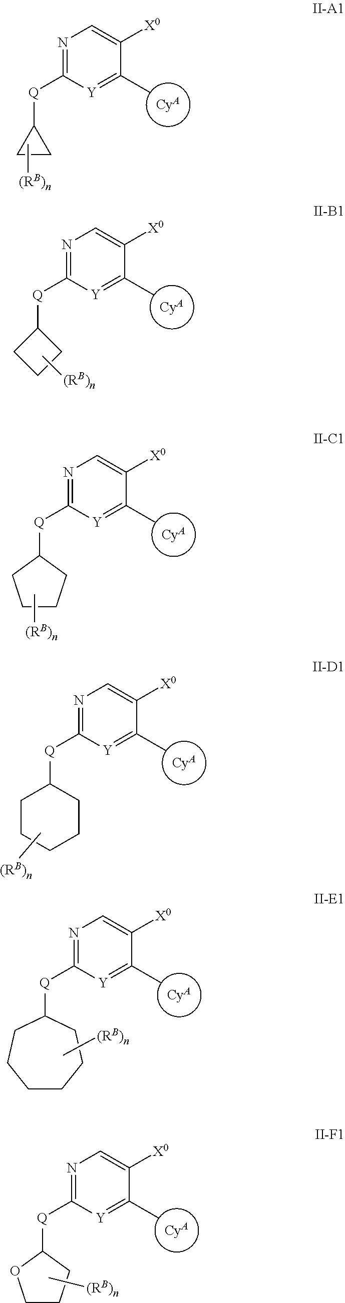

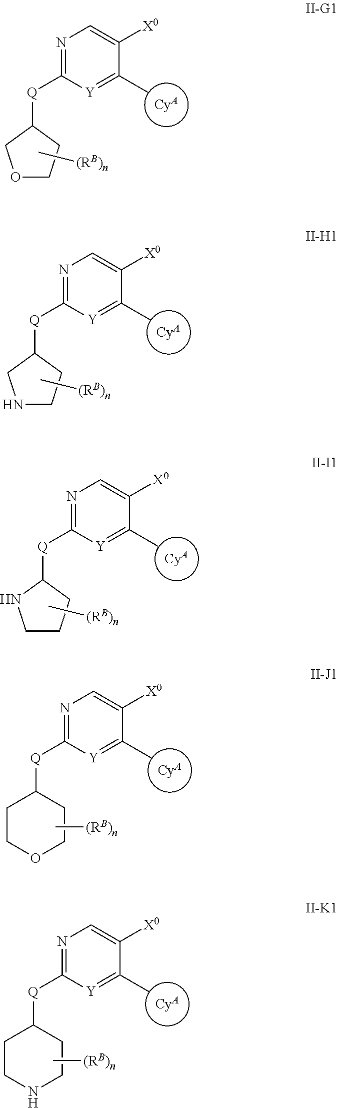

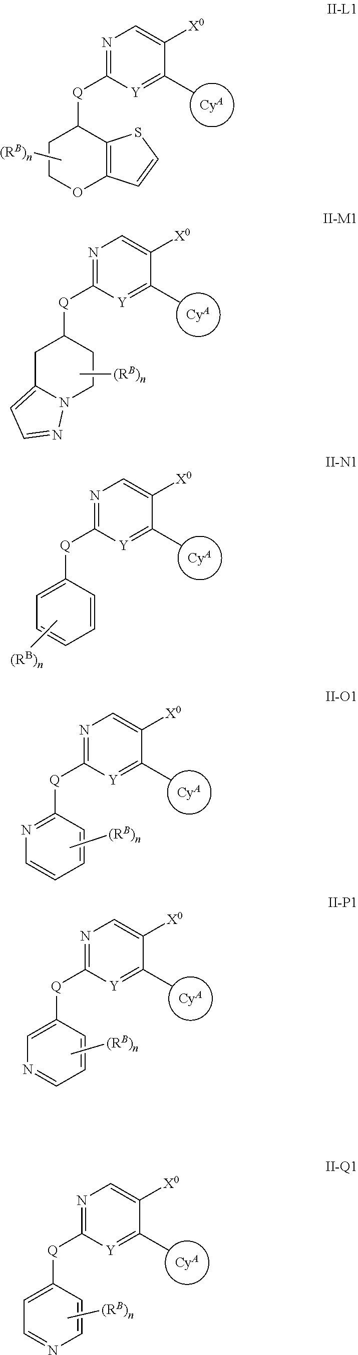

or a pharmaceutically acceptable salt thereof, wherein each of Cy^{A}, Q X⁰, Y, R^{B}, and n is as defined in embodiments and classes and subclasses herein.

In some embodiments, the present disclosure provides a compound of formula II-A2, II-B2, II-C2, II-D2, II-E2, II-F2, II-G2, and II-H2:

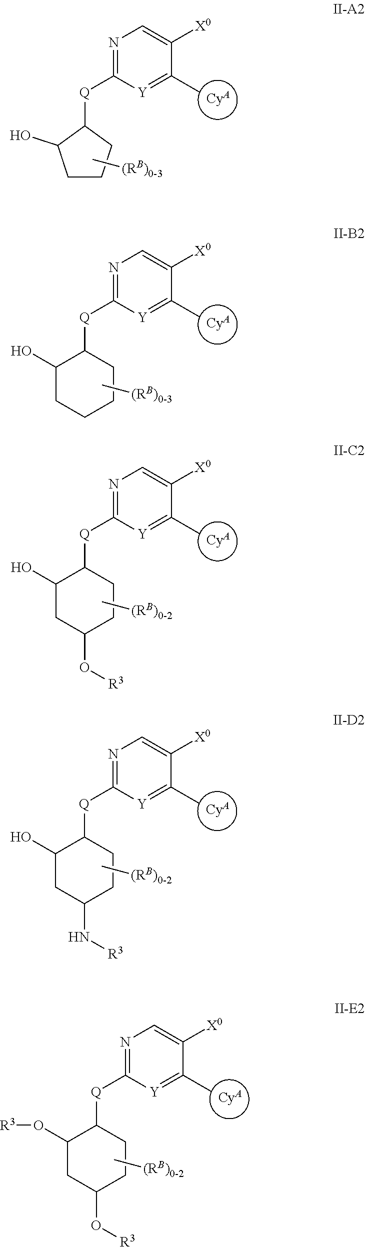

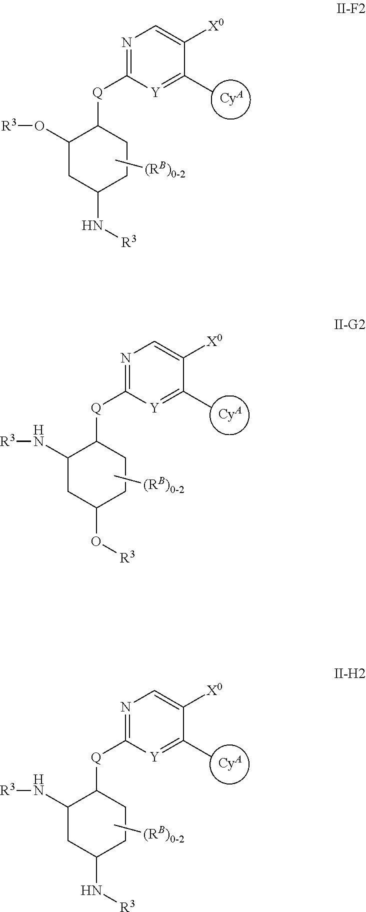

or a pharmaceutically acceptable salt thereof, wherein each of Cy^{A}, Q X⁰, Y, R^{B}, and R³ is as defined in embodiments and classes and subclasses herein.

In some embodiments, the present disclosure provides a compound of formula V, VI, VII, VIII, IX, X, XI, XII, XIII, XIV, XV, XVI, XVII, XVIII, XIX, XX, XXI, and XXII

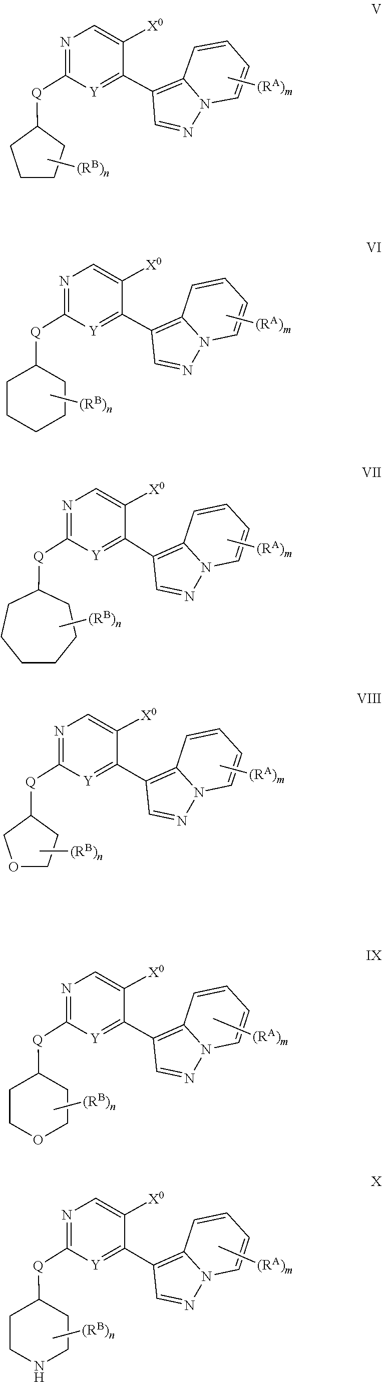

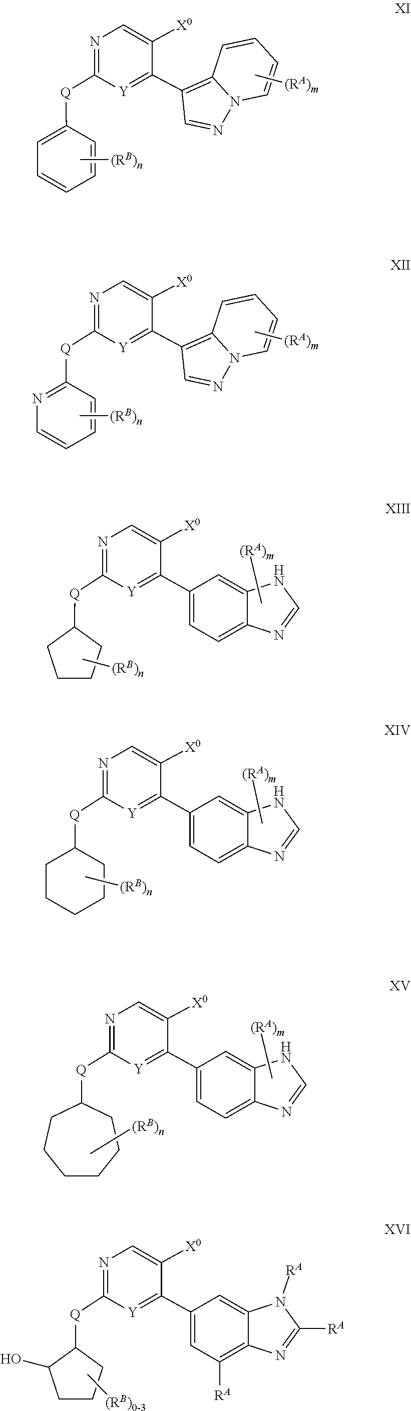

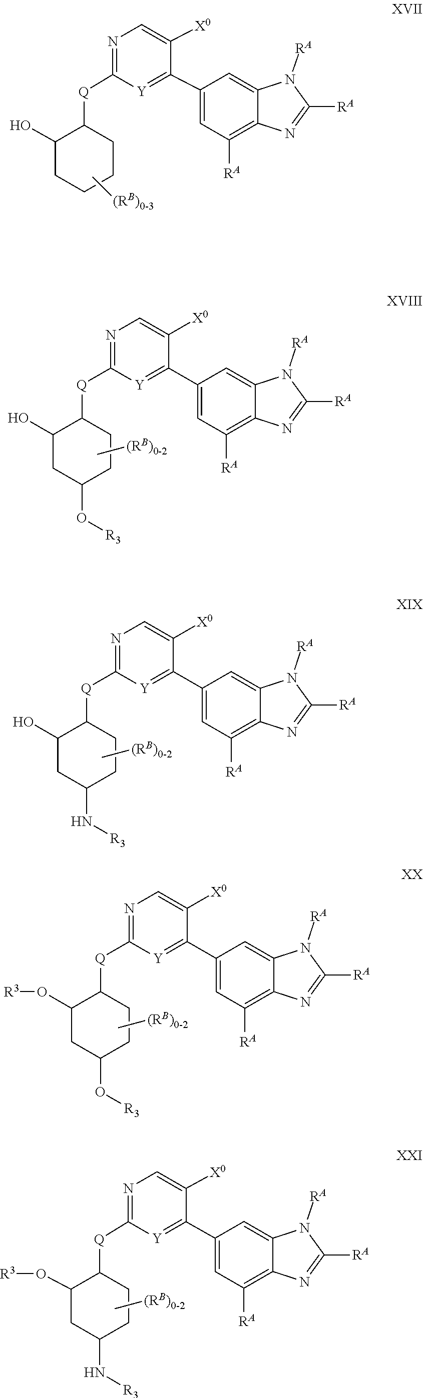

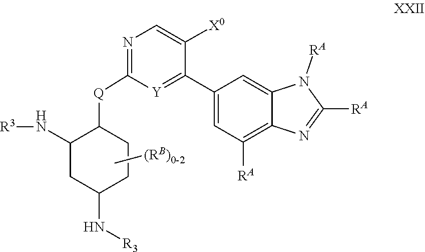

or a pharmaceutically acceptable salt thereof, wherein each of Q, X⁰, Y, R^{A}, R^{B}, R³, m, and n is as defined in embodiments and classes and subclasses herein.

In some embodiments, the present disclosure provides a compound of formula XXIII, XXIV, XXV, XXVI, and XXVII:

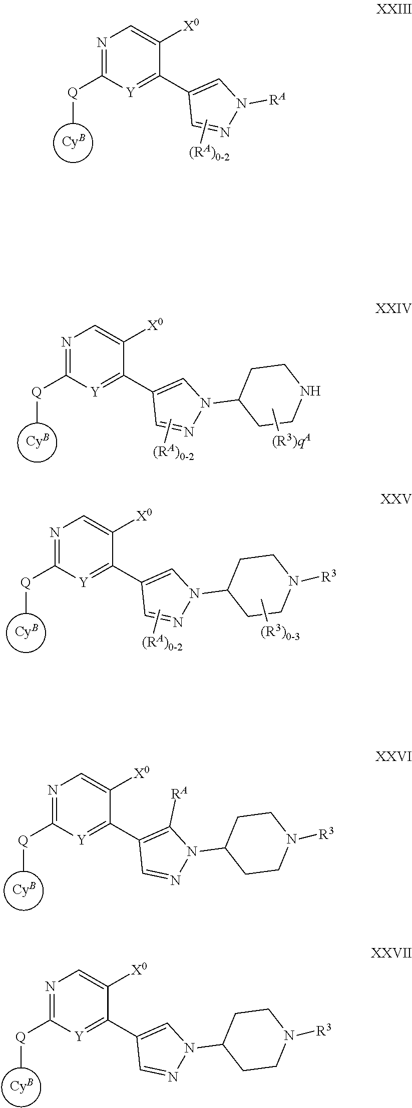

or a pharmaceutically acceptable salt thereof, wherein each of Cy^{B}, Q, X⁰, Y, R^{A}, R³, and q^{A} is as defined in embodiments and classes and subclasses herein.

In some embodiments, the present disclosure provides a compound of formula XXVIII, XXIX, XXX, XXXI, XXXII, XXXIII, XXXIV, XXXV, XXXVI, XXXVII, XXXVIII, and XXXIX:

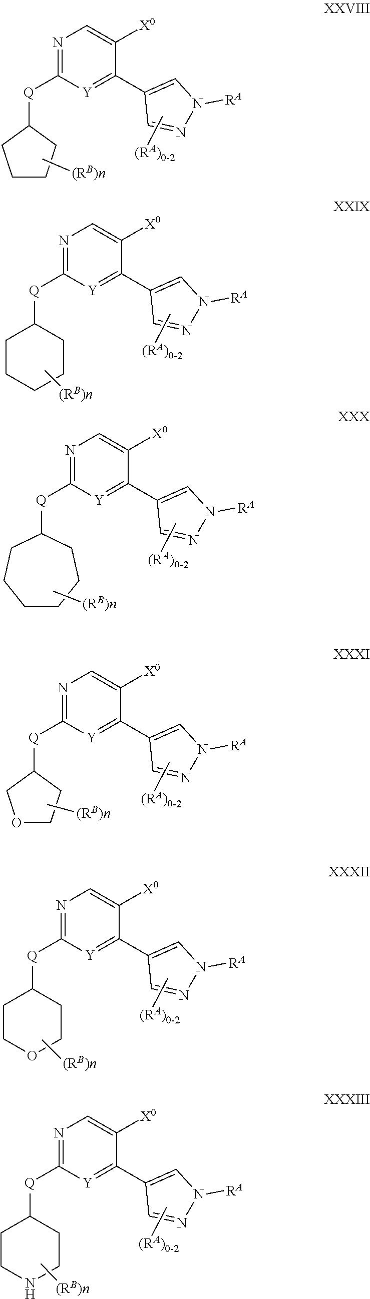

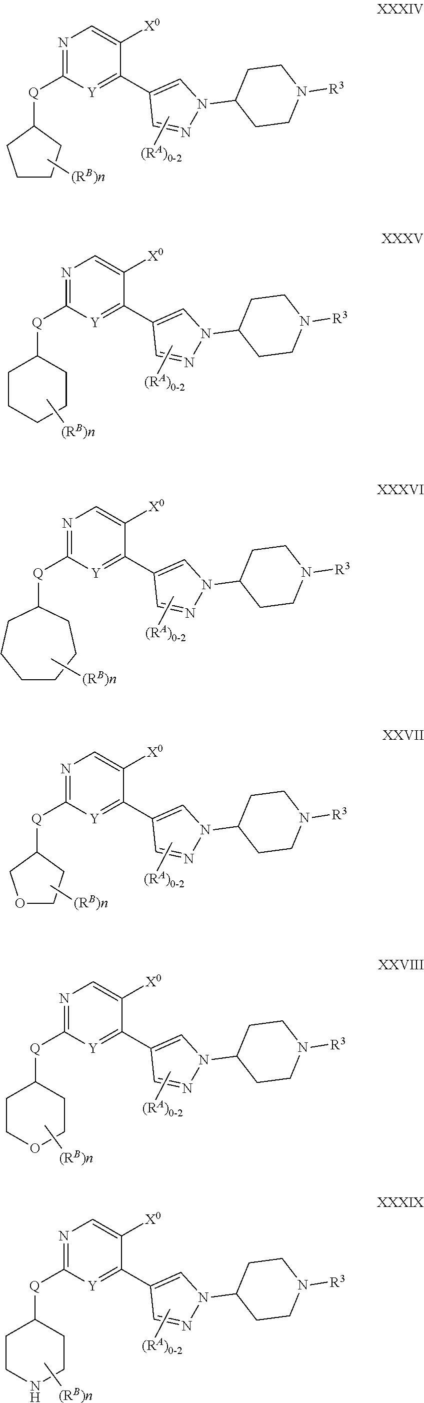

or a pharmaceutically acceptable salt thereof, wherein each of Q, X⁰, Y, R^{A}, R^{B}, R³, and n is as defined in embodiments and classes and subclasses herein.

In some embodiments, the present disclosure provides a compound of formula XL, XLI, XLII, and XLIII:

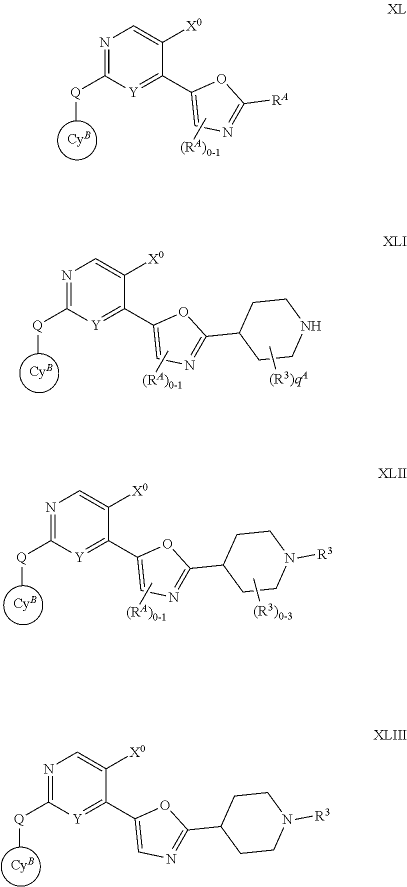

or a pharmaceutically acceptable salt thereof, wherein each of Cy^{B}, Q, X⁰, Y, R^{A}, R³, and q^{A} is as defined in embodiments and classes and subclasses herein.

In some embodiments, the present disclosure provides a compound of formula XLIV, XLV, XLVI, XLVII, XLVIII, XLIX, L, LI, LII, LIII, LIV, and LV:

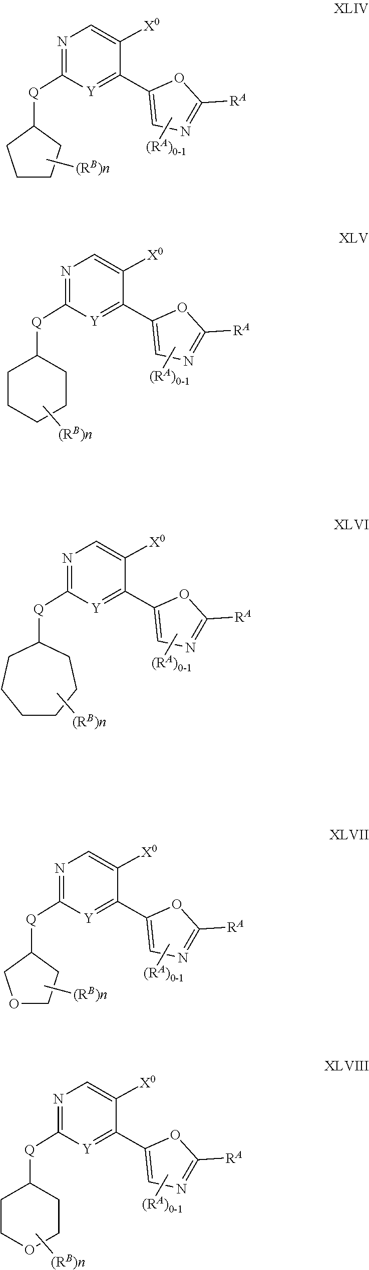

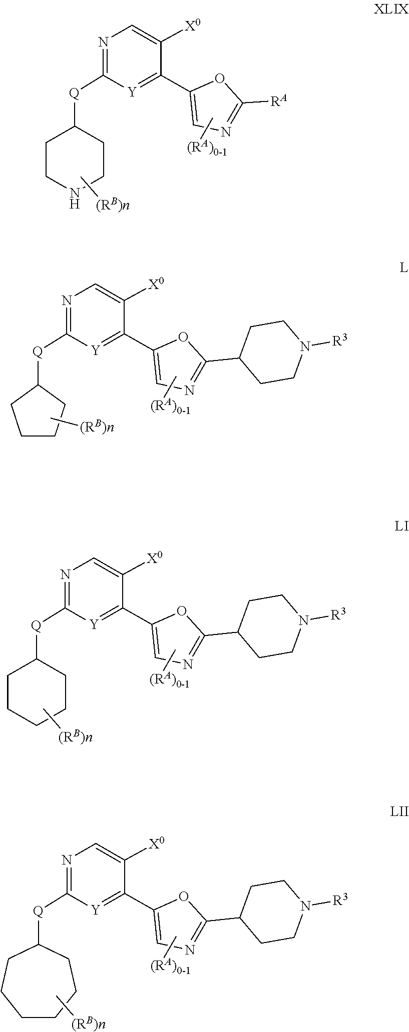

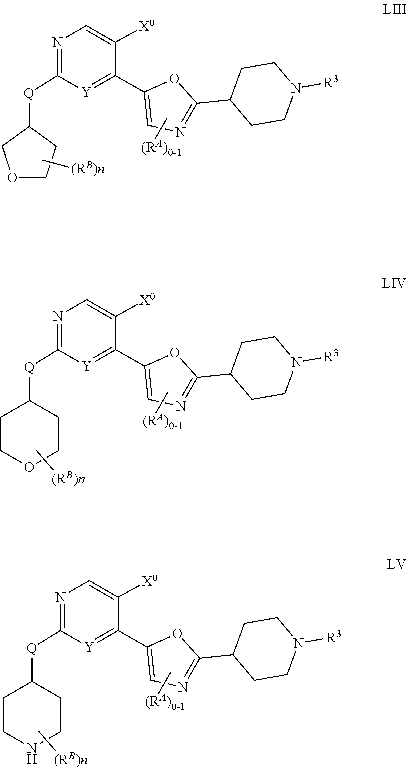

or a pharmaceutically acceptable salt thereof, wherein each of Q, X⁰, Y, R^{A}, R^{B}, R³, and n is as defined in embodiments and classes and subclasses herein.

In some embodiments, the present disclosure provides a compound of formula LVI, LVII, LVIII, and LIX:

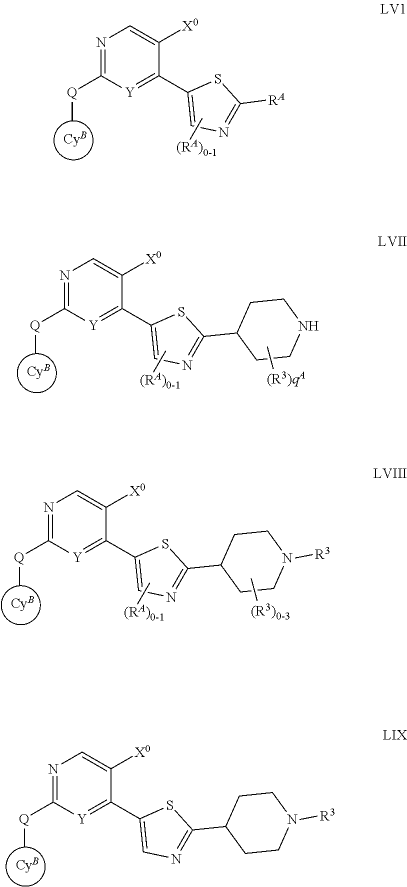

or a pharmaceutically acceptable salt thereof, wherein each of Cy^{B}, Q, X⁰, Y, R^{A}, R³, and q^{A} is as defined in embodiments and classes and subclasses herein.

In some embodiments, the present disclosure provides a compound of formula LX, LXI, LXII, LXIII, LXIV, LXV, LXVI, LXVII, LXVIII, LXIX, LXX, and LXXI:

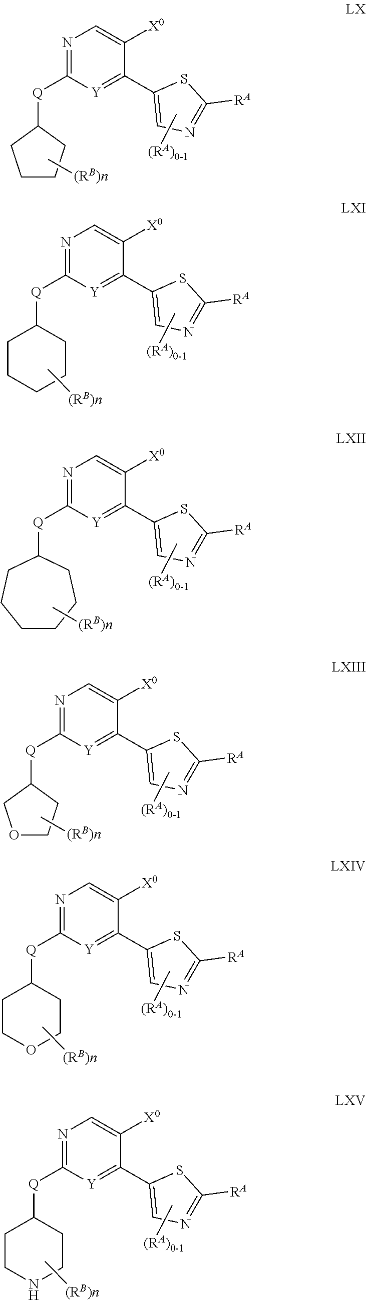

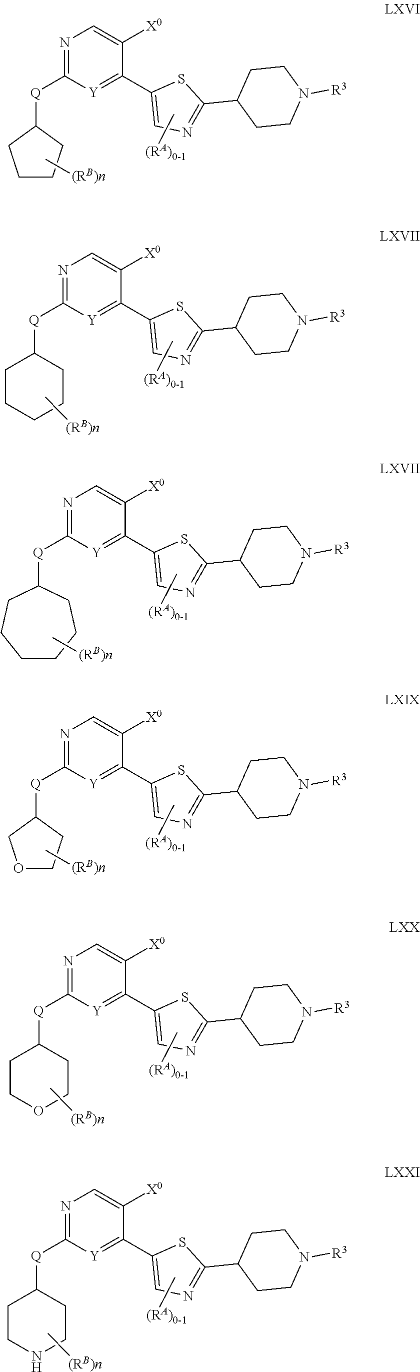

or a pharmaceutically acceptable salt thereof, wherein each of Q, X⁰, Y, R^{A}, R^{B}, R³, and n is as defined in embodiments and classes and subclasses herein.

In some embodiments, the present disclosure provides a compound of formula LXXII, LXXIII, LXXIV, and LXXV:

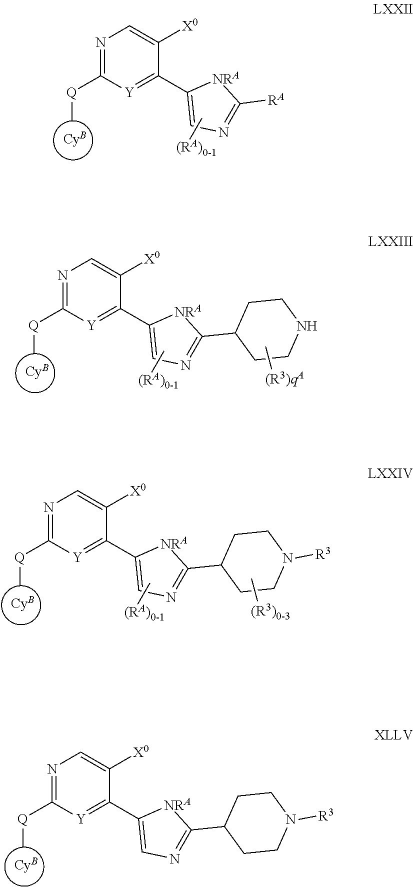

or a pharmaceutically acceptable salt thereof, wherein each of Cy^{B}, Q, X⁰, Y, R^{A}, R³, and q^{A} is as defined in embodiments and classes and subclasses herein.

In some embodiments, the present disclosure provides a compound of formula LXXVI, LXXVII, LXXVIII, LXXIX, LXXX, LXXXI, LXXXII, LXXXIII, LXXXIV, LXXXV, LXXXVI, and LXXXVII:

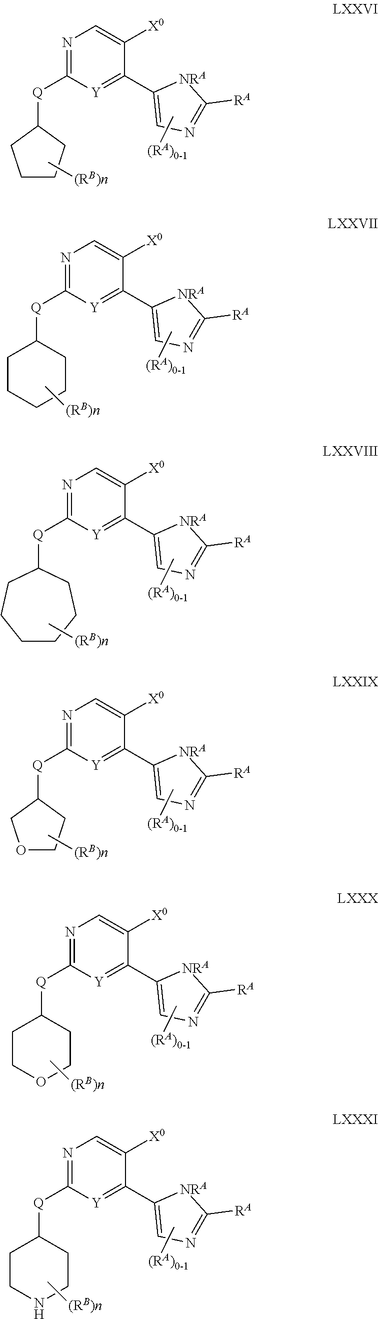

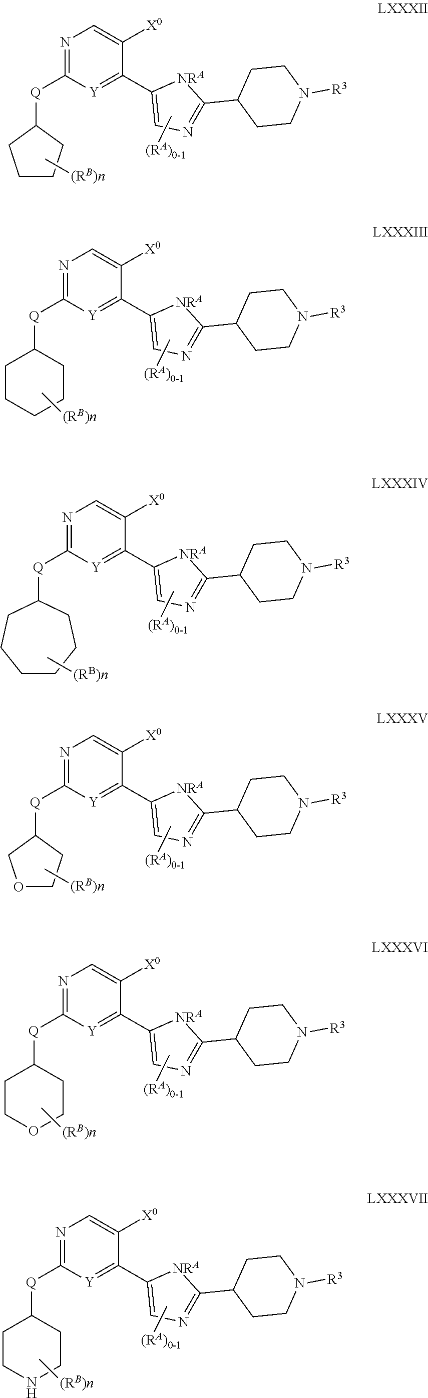

or a pharmaceutically acceptable salt thereof, wherein each of Q, X⁰, Y, R^{A}, R^{B}, R³, and n is as defined in embodiments and classes and subclasses herein.

In some embodiments, the present disclosure provides a compound of formula LXXXVIII, LXXXIX, XC, and XCI:

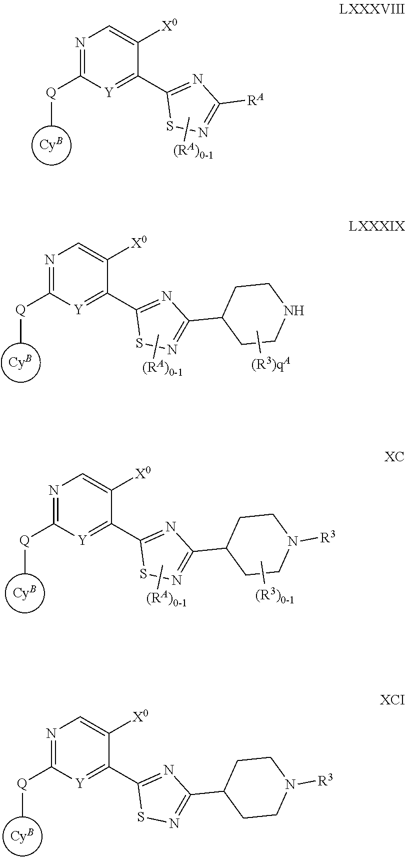

or a pharmaceutically acceptable salt thereof, wherein each of Cy^{B}, Q, X⁰, Y, R^{A}, R³, and q^{A} is as defined in embodiments and classes and subclasses herein.

As defined generally above, Cy^{A} is a phenyl; a 5-6 membered monocyclic heteroaryl ring having 1-4 heteroatoms independently selected from nitrogen, oxygen, phosphorus, and sulfur; an 8-11 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{A} is substituted with m instances of R^{A}.

In some embodiments, Cy^{A} is phenyl. In some embodiments, Cy^{A} is

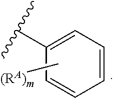

In some embodiments, Cy^{A} is a 3-8 membered saturated or partially unsaturated monocyclic carbocyclic ring, wherein Cy^{A} is substituted with m instances of R^{A}. In some embodiments, Cy^{A} is a 3-8 membered monocyclic saturated or partially unsaturated monocyclic carbocyclic ring, wherein Cy^{A} is substituted with m instances of R^{A}. In some embodiments, Cy^{A} is a cyclpropyl ring. In some embodiments, Cy^{A} is a cyclobutyl ring. In some embodiments, Cy^{A} is a cyclopentyl ring. In some embodiments, Cy^{A} is a cyclohexyl ring. In some embodiments, Cy^{A} is a cycloheptyl ring. In some embodiments, Cy^{A} is a cyclooctyl ring.

In some embodiments, Cy^{A} is

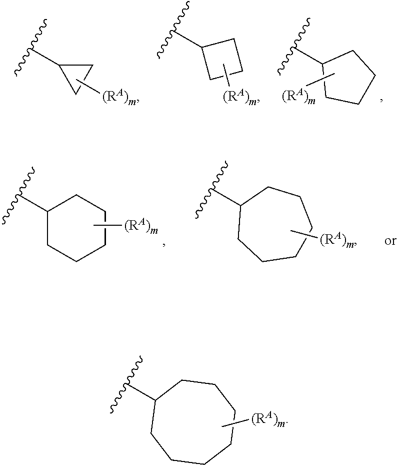

In some embodiments, Cy^{A} is

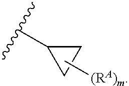

In some embodiments, Cy^{A} is

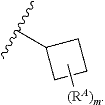

In some embodiments, Cy^{A} is

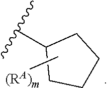

In some embodiments, Cy^{A} is

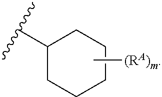

In some embodiments, Cy^{A} is

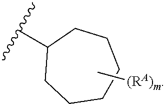

In some embodiments, Cy^{A} is

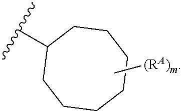

In some embodiments, Cy^{A} is a 5-6 membered monocyclic heteroaryl ring having 1-4 heteroatoms independently selected from nitrogen, oxygen, phosphorus, and sulfur, wherein Cy^{A} is substituted with m instances of R^{A}. In some embodiments, Cy^{A} is a 5-membered monocyclic heteroaryl ring having 1-4 heteroatoms independently selected from nitrogen, oxygen, phosphorus, and sulfur, wherein Cy^{A} is substituted with m instances of R^{A}. In some embodiments, Cy^{A} is a 6-membered monocyclic heteroaryl ring having 1-4 heteroatoms independently selected from nitrogen, oxygen, phosphorus, and sulfur, wherein Cy^{A} is substituted with m instances of R^{A}.

In some embodiments, Cy^{A} is

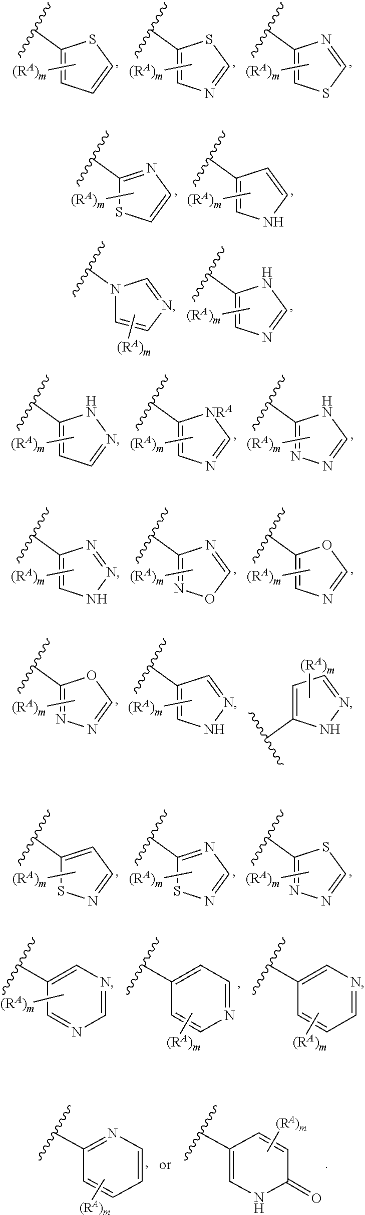

In some embodiments, Cy^{A} is

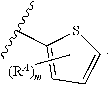

In some embodiments, Cy^{A} is

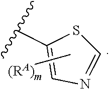

In some embodiments, Cy^{A} is

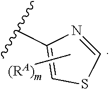

In some embodiments, Cy^{A} is

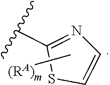

In some embodiments, Cy^{A} is

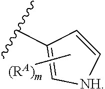

In some embodiments, Cy^{A} is

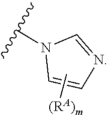

In some embodiments, Cy^{A} is

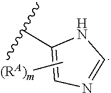

In some embodiments, Cy^{A} is

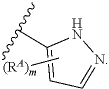

In some embodiments, Cy^{A} is

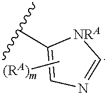

In some embodiments, Cy^{A} is

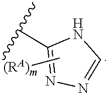

In some embodiments, Cy^{A} is

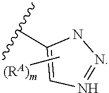

In some embodiments, Cy^{A} is

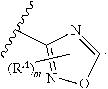

In some embodiments, Cy^{A} is

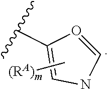

In some embodiments, Cy^{A} is

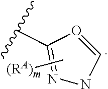

In some embodiments, Cy^{A} is

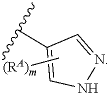

In some embodiments, Cy^{A} is

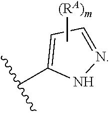

In some embodiments, Cy^{A} is

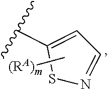

In some embodiments, Cy^{A} is

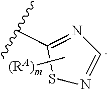

In some embodiments, Cy^{A} is

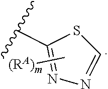

In some embodiments, Cy^{A} is

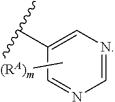

In some embodiments, Cy^{A} is

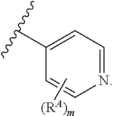

In some embodiments, Cy^{A} is

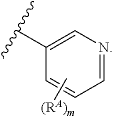

In some embodiments, Cy^{A} is

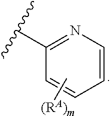

In some embodiments, Cy^{A} is

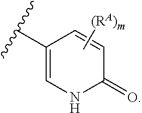

In some embodiments, Cy^{A} is

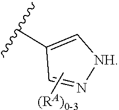

In some embodiments, Cy^{A} is

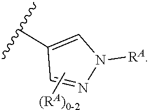

In some embodiments, Cy^{A} is

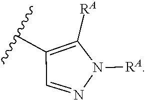

In some embodiments, Cy^{A} is

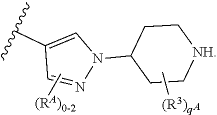

In some embodiments, Cy^{A} is

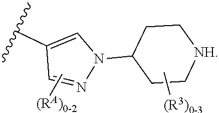

In some embodiments, Cy^{A} is

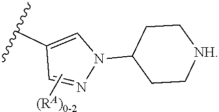

In some embodiments, Cy^{A} is

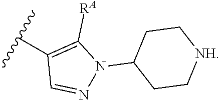

In some embodiments, Cy^{A} is

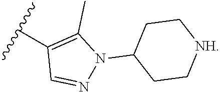 [SMILES: *c1cnn(C2CCNCC2)c1C]

In some embodiments, Cy^{A} is

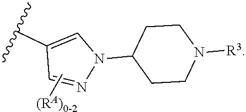

In some embodiments, Cy^{A} is

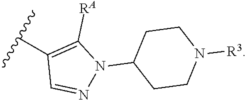

In some embodiments, Cy^{A} is

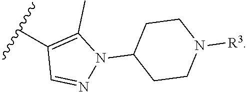

In some embodiments, Cy^{A} is

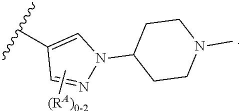

In some embodiments, Cy^{A} is

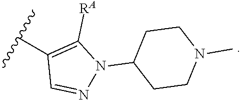

In some embodiments, Cy^{A} is

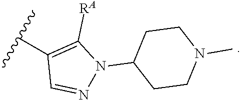

In some embodiments, Cy^{A} is

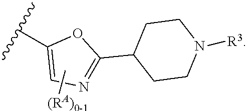

In some embodiments, Cy^{A} is

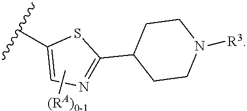

In some embodiments, Cy^{A} is

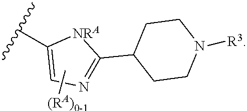

In some embodiments, Cy^{A} is

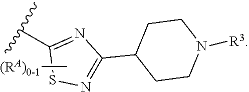

In some embodiments, Cy^{A} is

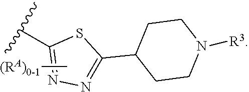

In some embodiments, Cy^{A} is an 8-11 membered bicyclic heteroaryl ring having 1-4 heteroatoms independently selected from nitrogen, oxygen, phosphorus, and sulfur, wherein Cy^{A} is substituted with m instances of R^{A}. In some embodiments, Cy^{A} is an 8-membered bicyclic heteroaryl ring having 1-4 heteroatoms independently selected from nitrogen, oxygen, phosphorus, and sulfur, wherein Cy^{A} is substituted with m instances of R^{A}. In some embodiments, Cy^{A} is a 9-membered bicyclic heteroaryl ring having 1-4 heteroatoms independently selected from nitrogen, oxygen, phosphorus, and sulfur, wherein Cy^{A} is substituted with m instances of R^{A}. In some embodiments, Cy^{A} is a 10-membered bicyclic heteroaryl ring having 1-4 heteroatoms independently selected from nitrogen, oxygen, phosphorus, and sulfur, wherein Cy^{A} is substituted with m instances of R^{A}. In some embodiments, Cy^{A} is an 11-membered bicyclic heteroaryl ring having 1-4 heteroatoms independently selected from nitrogen, oxygen, phosphorus, and sulfur, wherein Cy^{A} is substituted with m instances of R^{A}.

In some embodiments, Cy^{A} is

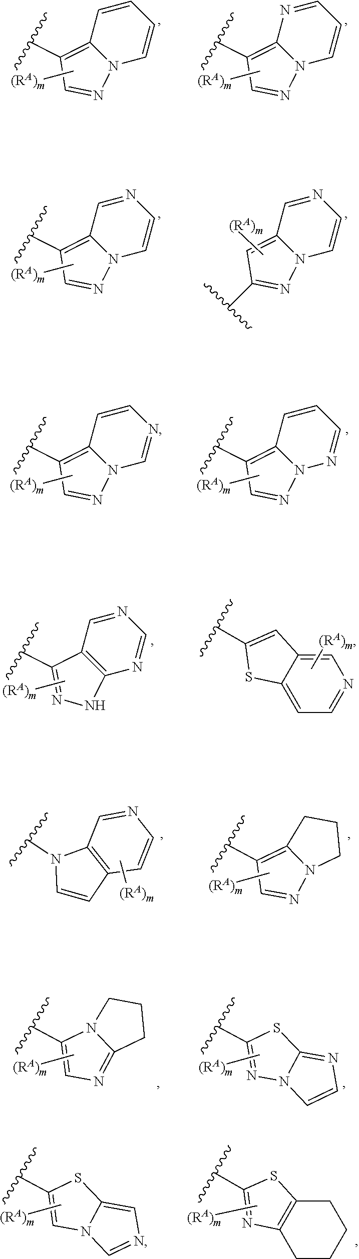

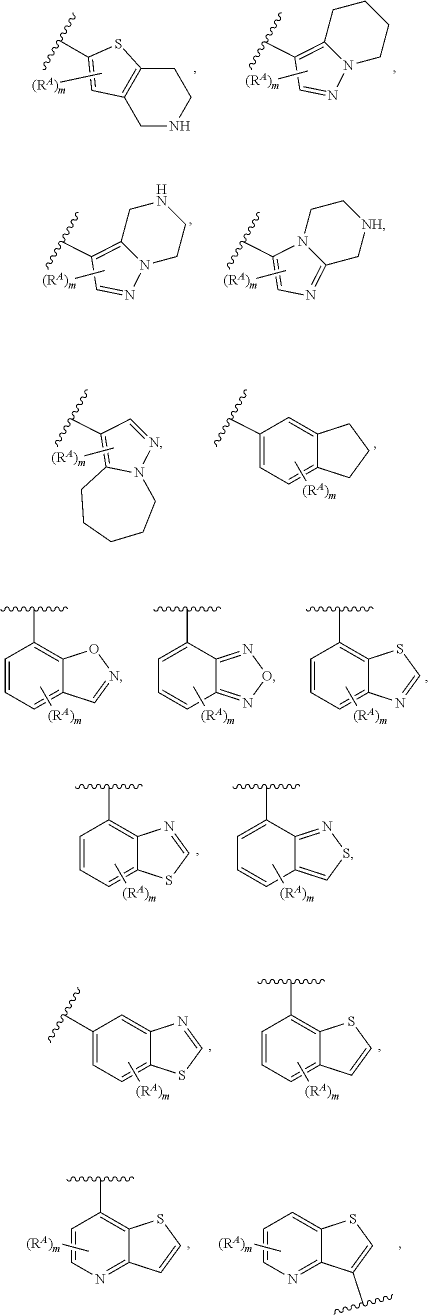

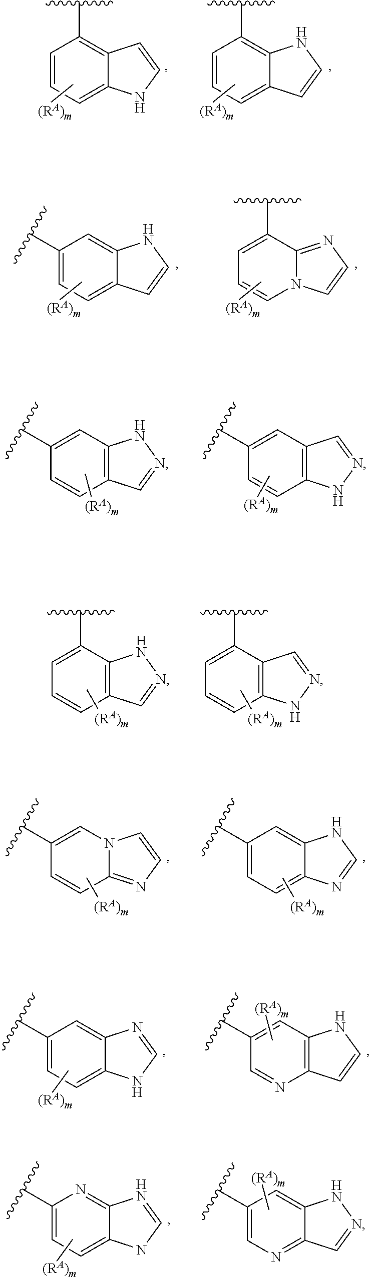

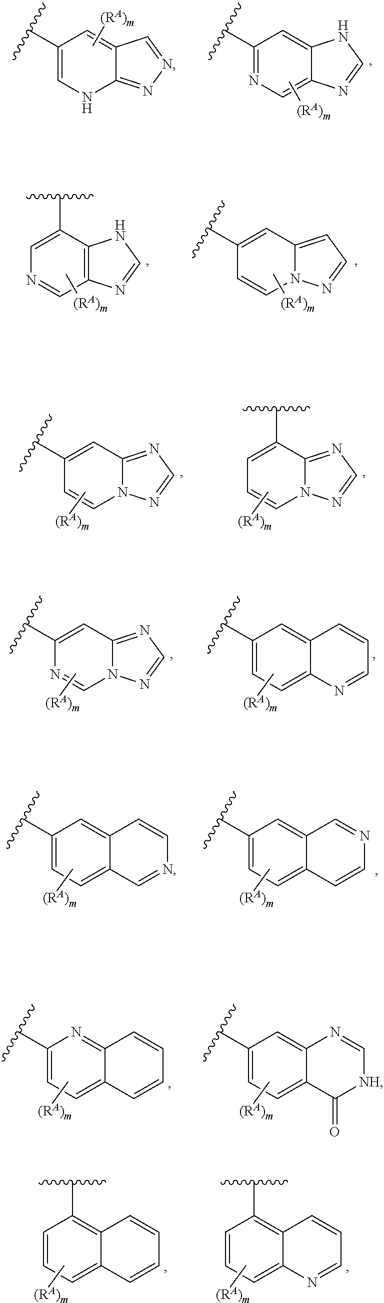

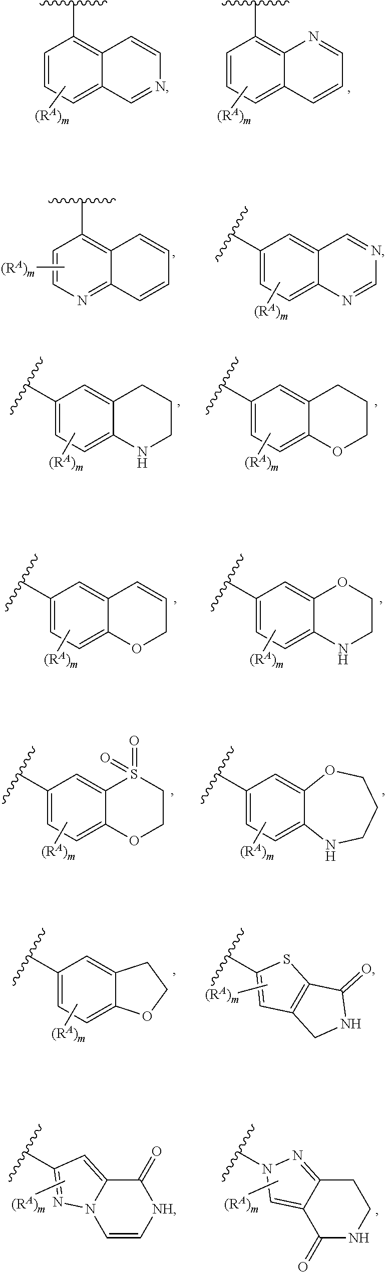

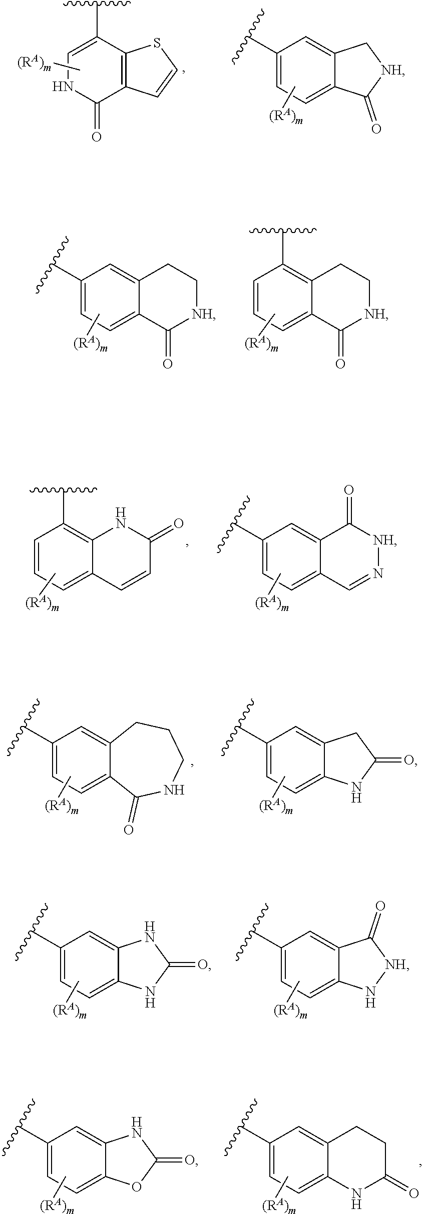

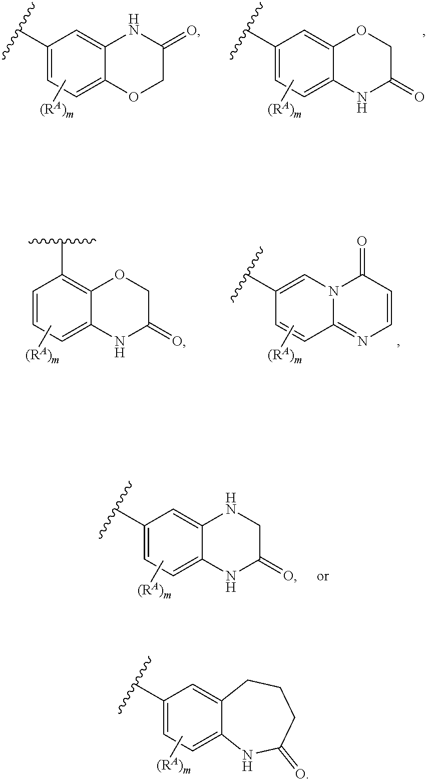

In some embodiments, Cy^{A} is

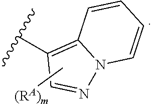

In some embodiments, Cy^{A} is

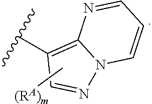

In some embodiments, Cy^{A} is

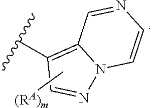

In some embodiments, Cy^{A} is

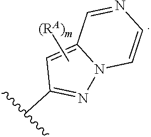

In some embodiments, Cy^{A} is

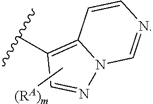

In some embodiments, Cy^{A} is

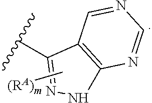

In some embodiments, Cy^{A} is

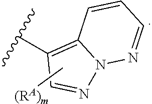

In some embodiments, Cy^{A} is

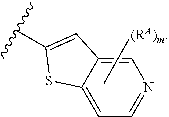

In some embodiments, Cy^{A} is

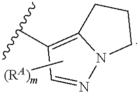

In some embodiments, Cy^{A} is

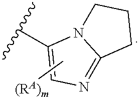

In some embodiments, Cy^{A} is

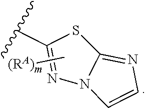

In some embodiments, Cy^{A} is

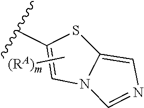

In some embodiments, Cy^{A} is

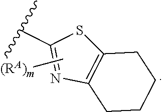

In some embodiments, Cy^{A} is

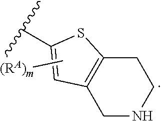

In some embodiments, Cy^{A} is

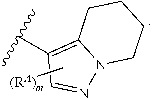

In some embodiments, Cy^{A} is

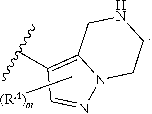

In some embodiments, Cy^{A} is

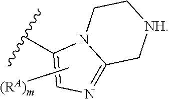

In some embodiments, Cy^{A} is

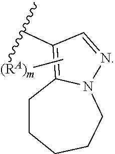

In some embodiments, Cy^{A} is

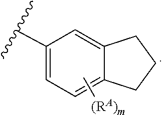

In some embodiments, Cy^{A} is

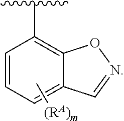

In some embodiments, Cy^{A} is

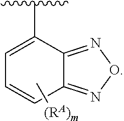

In some embodiments, Cy^{A} is

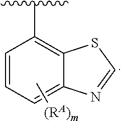

In some embodiments, Cy^{A} is

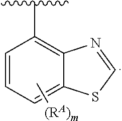

In some embodiments, Cy^{A} is

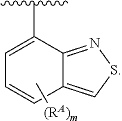

In some embodiments, Cy^{A} is

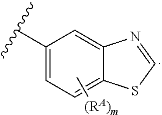

In some embodiments, Cy^{A} is

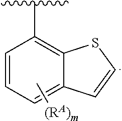

In some embodiments, Cy^{A} is

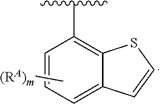

In some embodiments, Cy^{A} is

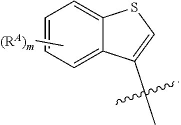

In some embodiments, Cy^{A} is

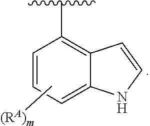

In some embodiments, Cy^{A} is

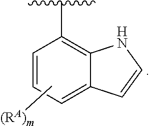

In some embodiments, Cy^{A} is

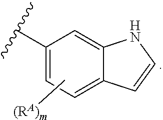

In some embodiments, Cy^{A} is

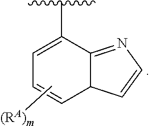

In some embodiments, Cy^{A} is

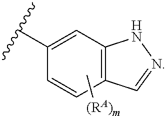

In some embodiments, Cy^{A} is

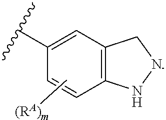

In some embodiments, Cy^{A} is

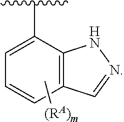

In some embodiments, Cy^{A} is

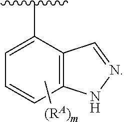

In some embodiments, Cy^{A} is

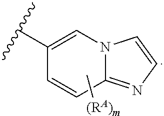

In some embodiments, Cy^{A} is

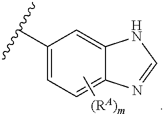

In some embodiments, Cy^{A} is

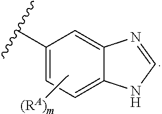

In some embodiments, Cy^{A} is

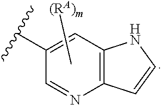

In some embodiments, Cy^{A} is

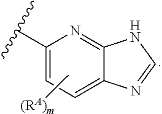

In some embodiments, Cy^{A} is

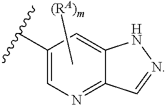

In some embodiments, Cy^{A} is

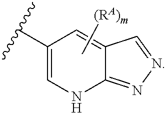

In some embodiments, Cy^{A} is

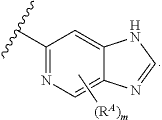

In some embodiments, Cy^{A} is

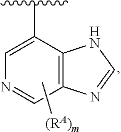

In some embodiments, Cy^{A} is

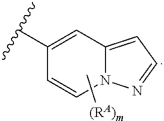

In some embodiments, Cy^{A} is

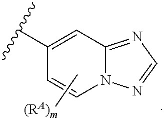

In some embodiments, Cy^{A} is

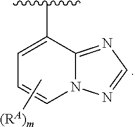

In some embodiments, Cy^{A} is

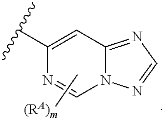

In some embodiments, Cy^{A} is

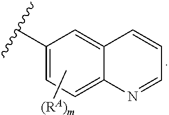

In some embodiments, Cy^{A} is

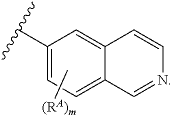

In some embodiments, Cy^{A} is

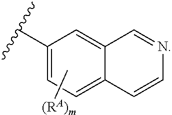

In some embodiments, Cy^{A} is

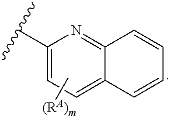

In some embodiments, Cy^{A} is

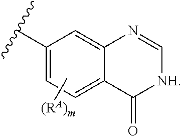

In some embodiments, Cy^{A} is

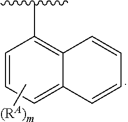

In some embodiments, Cy^{A} is

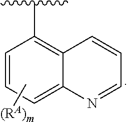

In some embodiments, Cy^{A} is

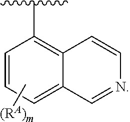

In some embodiments, Cy^{A} is

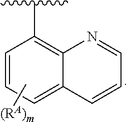

In some embodiments, Cy^{A} is

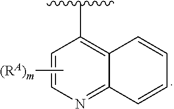

In some embodiments, Cy^{A} is

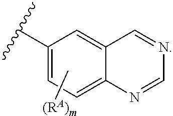

In some embodiments, Cy^{A} is

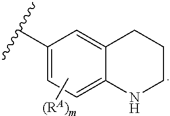

In some embodiments, Cy^{A} is

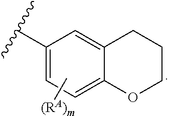

In some embodiments, Cy^{A} is

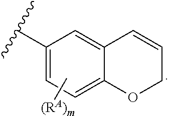

In some embodiments, Cy^{A} is

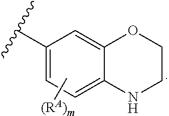

In some embodiments, Cy^{A} is

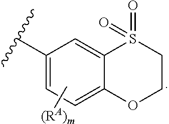

In some embodiments, Cy^{A} is

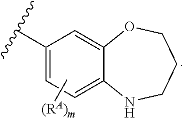

In some embodiments, Cy^{A} is

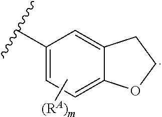

In some embodiments, Cy^{A} is

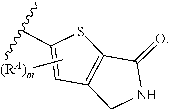

In some embodiments, Cy^{A} is

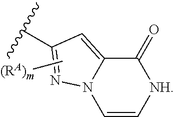

In some embodiments, Cy^{A} is

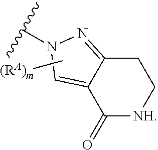

In some embodiments, Cy^{A} is

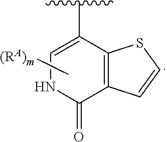

In some embodiments, Cy^{A} is

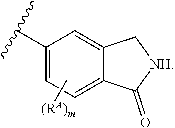

In some embodiments, Cy^{A} is

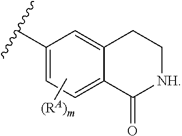

In some embodiments, Cy^{A} is

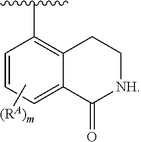

In some embodiments, Cy^{A} is

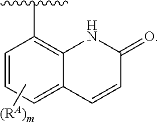

In some embodiments, Cy^{A} is

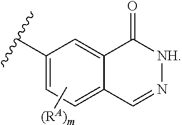

In some embodiments, Cy^{A} is

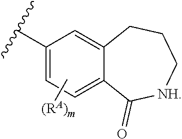

In some embodiments, Cy^{A} is

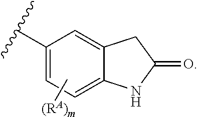

In some embodiments, Cy^{A} is

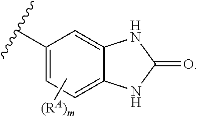

In some embodiments, Cy^{A} is

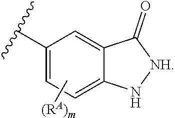

In some embodiments, Cy^{A} is

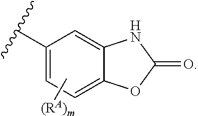

In some embodiments, Cy^{A} is

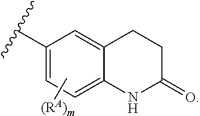

In some embodiments, Cy^{A} is

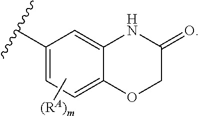

In some embodiments, Cy^{A} is

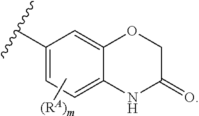

In some embodiments, Cy^{A} is

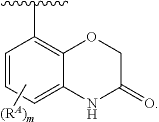

In some embodiments, Cy^{A} is

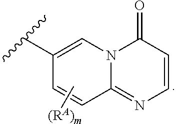

In some embodiments, Cy^{A} is

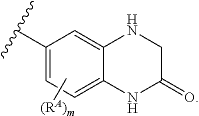

In some embodiments, Cy^{A} is

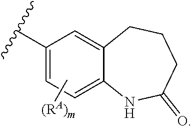

In some embodiments, Cy^{A} is

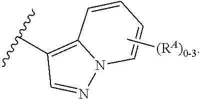

In some embodiments, Cy^{A} is

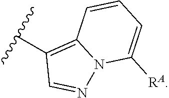

In some embodiments, Cy^{A} is

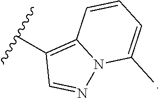 [SMILES: *c1cnn2c(C)cccc12]

In some embodiments, Cy^{A} is

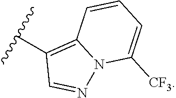 [SMILES: *c1cnn2c(C(F)(F)F)cccc12]

In some embodiments, Cy^{A} is

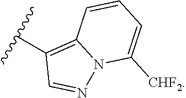 [SMILES: *c1cnn2c(C(F)F)cccc12]

In some embodiments, Cy^{A} is

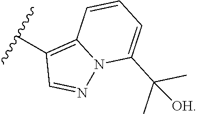 [SMILES: *c1cnn2c(C(C)(C)O)cccc12]

In some embodiments, Cy^{A} is

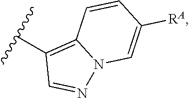

In some embodiments, Cy^{A} is

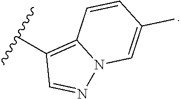 [SMILES: *c1cnn2cc(C)ccc12]

In some embodiments, Cy^{A} is

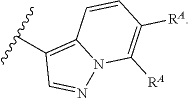

In some embodiments, Cy^{A} is

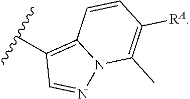

In some embodiments, Cy^{A} is

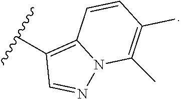 [SMILES: *c1cnn2c(C)c(C)ccc12]

In some embodiments, Cy^{A} is

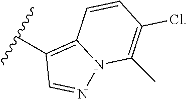 [SMILES: *c1cnn2c(C)c(Cl)ccc12]

In some embodiments, Cy^{A} is

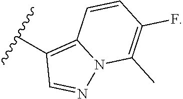 [SMILES: *c1cnn2c(C)c(F)ccc12]

In some embodiments, Cy^{A} is

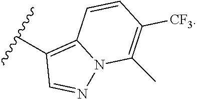 [SMILES: *c1cnn2c(C)c(C(F)(F)F)ccc12]

In some embodiments, Cy^{A} is

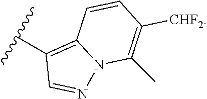 [SMILES: *c1cnn2c(C)c(C(F)F)ccc12]

In some embodiments, Cy^{A} is

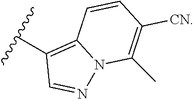 [SMILES: *c1cnn2c(C)c(C#N)ccc12]

In some embodiments, Cy^{A} is

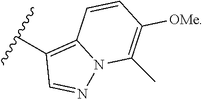 [SMILES: *c1cnn2c(C)c(OC)ccc12]

In some embodiments, Cy^{A} is

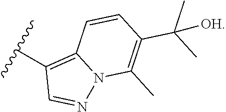 [SMILES: *c1cnn2c(C)c(C(C)(C)O)ccc12]

In some embodiments, Cy^{A} is

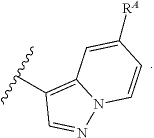

In some embodiments, Cy^{A} is

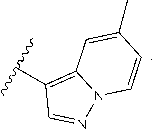 [SMILES: *c1cnn2ccc(C)cc12]

In some embodiments, Cy^{A} is

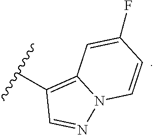 [SMILES: *c1cnn2ccc(F)cc12]

In some embodiments, Cy^{A} is

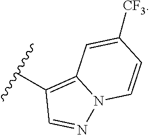 [SMILES: *c1cnn2ccc(C(F)(F)F)cc12]

In some embodiments, Cy^{A} is

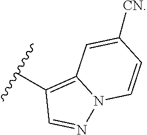 [SMILES: *c1cnn2ccc(C#N)cc12]

In some embodiments, Cy^{A} is

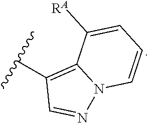

In some embodiments, Cy^{A} is

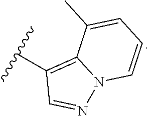 [SMILES: *c1cnn2cccc(C)c12]

In some embodiments, Cy^{A} is

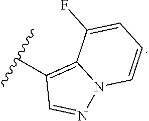 [SMILES: *c1cnn2cccc(F)c12]

In some embodiments, Cy^{A} is

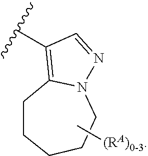

In some embodiments, Cy^{A} is

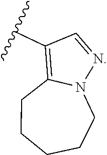 [SMILES: *c1cnn2c1CCCCC2]

In some embodiments, Cy^{A} is

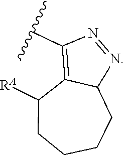

In some embodiments, Cy^{A} is

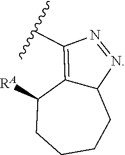

In some embodiments, Cy^{A} is

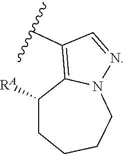

In some embodiments, Cy^{A} is

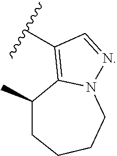 [SMILES: *c1cnn2c1[C@H](C)CCCC2]

In some embodiments, Cy^{A} is

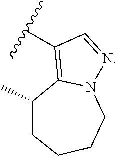 [SMILES: *c1cnn2c1[C@@H](C)CCCC2]

In some embodiments, Cy^{A} is

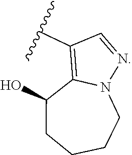 [SMILES: *c1cnn2c1[C@H](O)CCCC2]

In some embodiments, Cy^{A} is

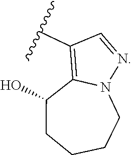 [SMILES: *c1cnn2c1[C@@H](O)CCCC2]

In some embodiments, Cy^{A} is

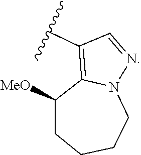 [SMILES: *c1cnn2c1[C@H](OC)CCCC2]

In some embodiments, Cy^{A} is

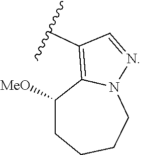 [SMILES: *c1cnn2c1[C@@H](OC)CCCC2]

In some embodiments, Cy^{A} is

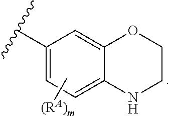

In some embodiments, Cy^{A} is

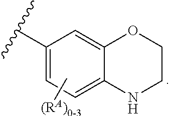

In some embodiments, Cy^{A} is

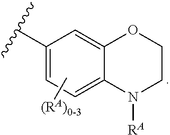

In some embodiments, Cy^{A} is

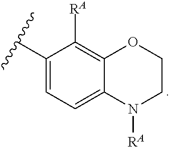

In some embodiments, Cy^{A} is

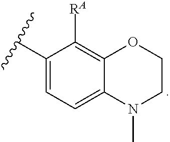

In some embodiments, Cy^{A} is

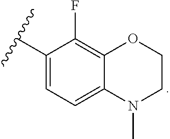 [SMILES: *c1ccc2c(c1F)OCCN2C]

In some embodiments, Cy^{A} is

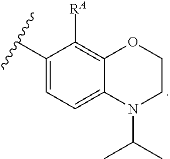

In some embodiments, Cy^{A} is

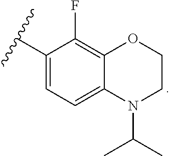 [SMILES: *c1ccc2c(c1F)OCCN2C(C)C]

In some embodiments, Cy^{A} is

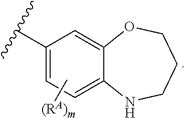

In some embodiments, Cy^{A} is

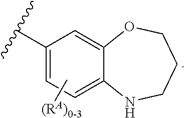

In some embodiments, Cy^{A} is

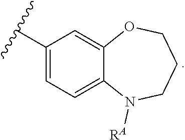

In some embodiments, Cy^{A} is

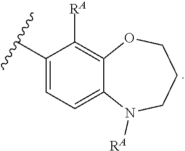

In some embodiments, Cy^{A} is

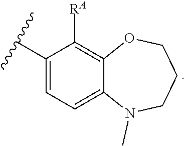

In some embodiments, Cy^{A} is

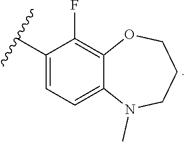 [SMILES: *c1ccc2c(c1F)OCCCN2C]

In some embodiments, Cy^{A} is

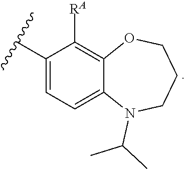

In some embodiments, Cy^{A}

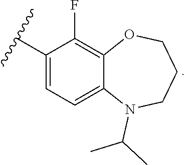 [SMILES: *c1ccc2c(c1F)OCCCN2C(C)C]

In some embodiments, Cy^{A} is

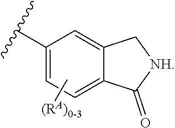

In some embodiments, Cy^{A} is

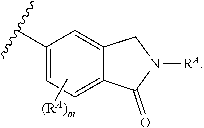

In some embodiments, Cy^{A} is

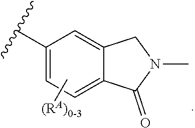

In some embodiments, Cy^{A} is

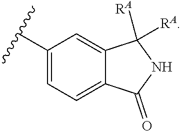

In some embodiments, Cy^{A} is

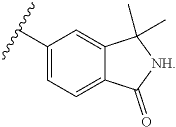 [SMILES: *c1ccc2c(c1)C(C)(C)NC2=O]

In some embodiments, Cy^{A} is an 8-11 membered bicyclic heteroaryl ring having 1-4 heteroatoms independently selected from nitrogen, oxygen, phosphorus, and sulfur, wherein Cy^{A} is substituted with at least 2 instances of R^{A}, wherein said 2 instances of R^{A} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³.

In some embodiments, Cy^{A} is

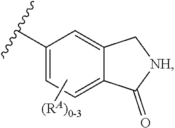

wherein two instances of R^{A} are taken together with their intervening atoms to form a 4-8 membered saturated, partially unsaturated or aromatic ring having 0-4 heteroatoms independently selected from nitrogen, oxygen, and sulfur, wherein said ring is substituted with r instances of R³.

In some embodiments, Cy^{A} is

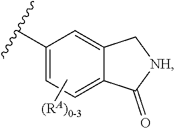

wherein two instances of R^{A} are taken together with their intervening atoms to form a 4-8 membered saturated ring, wherein said ring is substituted with r instances of R³. In some embodiments, Cy^{A} is

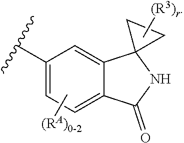

In some embodiments, Cy^{A} is

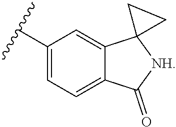 [SMILES: *c1ccc2c(c1)C1(CC1)NC2=O]

In some embodiments, Cy^{A}

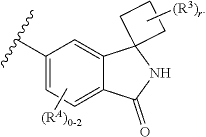

In some embodiments, Cy^{A} is

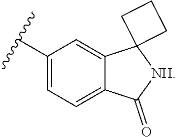 [SMILES: *c1ccc2c(c1)C1(CCC1)NC2=O]

In some embodiments, Cy^{A} is

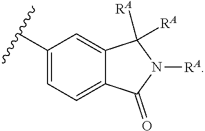

In some embodiments, Cy^{A} is

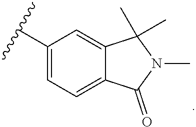 [SMILES: *c1ccc2c(c1)C(C)(C)N(C)C2=O]

In some embodiments, Cy^{A} is

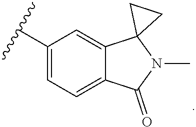 [SMILES: *c1ccc2c(c1)C1(CC1)N(C)C2=O]

In some embodiments, Cy^{A} is

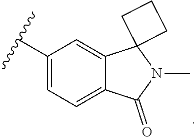 [SMILES: *c1ccc2c(c1)C1(CCC1)N(C)C2=O]

In some embodiments, Cy^{A} is

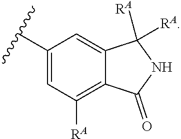

In some embodiments, Cy^{A} is

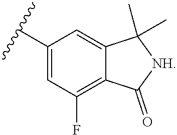 [SMILES: *c1cc(F)c2c(c1)C(C)(C)NC2=O]

In some embodiments, Cy^{A} is

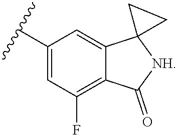 [SMILES: *c1cc(F)c2c(c1)C1(CC1)NC2=O]

In some embodiments, Cy^{A} is

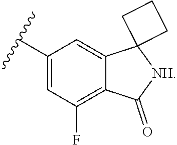 [SMILES: *c1cc(F)c2c(c1)C1(CCC1)NC2=O]

In some embodiments, Cy^{A} is

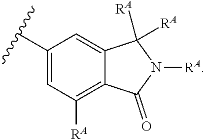

In some embodiments, Cy^{A} is

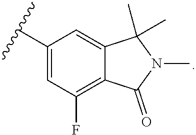 [SMILES: *c1cc(F)c2c(c1)C(C)(C)N(C)C2=O]

In some embodiments, Cy^{A} is

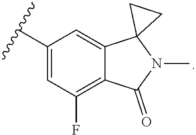 [SMILES: *c1cc(F)c2c(c1)C1(CC1)N(C)C2=O]

In some embodiments, Cy^{A} is

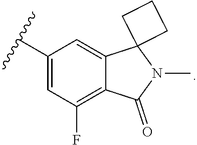 [SMILES: *c1cc(F)c2c(c1)C1(CCC1)N(C)C2=O]

In some embodiments, Cy^{A} is

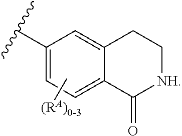

In some embodiments, Cy^{A} is

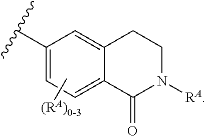

In some embodiments, Cy^{A} is

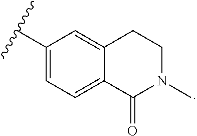 [SMILES: *c1ccc2c(c1)CCN(C)C2=O]

In some embodiments, Cy^{A} is

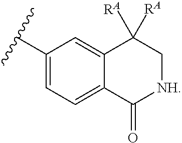

In some embodiments, Cy^{A} is

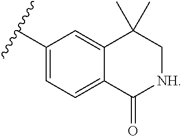 [SMILES: *c1ccc2c(c1)C(C)(C)CNC2=O]

In some embodiments, Cy^{A} is

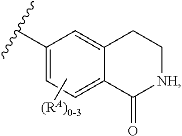

wherein two instances of R^{A} are taken together with their intervening atoms to form a 4-8 membered saturated, partially unsaturated or aromatic ring having 0-4 heteroatoms independently selected from nitrogen, oxygen, and sulfur, wherein said ring is substituted with r instances of R³.

In some embodiments, Cy^{A} is

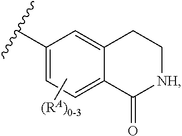

wherein two instances of R^{A} are taken together with their intervening atoms to form a 4-8 membered saturated ring, wherein said ring is substituted with r instances of R³. In some embodiments, Cy^{A} is

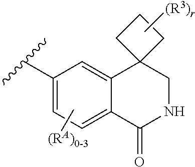

In some embodiments, Cy^{A} is

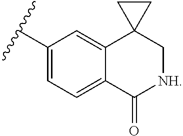 [SMILES: *c1ccc2c(c1)C1(CC1)CNC2=O]

In some embodiments, Cy^{A} is

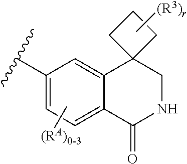

In some embodiments, Cy^{A} is

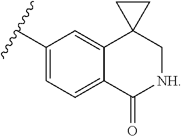 [SMILES: *c1ccc2c(c1)C1(CC1)CNC2=O]

In some embodiments, Cy^{A} is

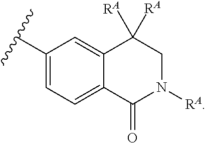

In some embodiments, Cy^{A} is

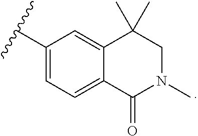 [SMILES: *c1ccc2c(c1)C(C)(C)CN(C)C2=O]

In some embodiments, Cy^{A} is

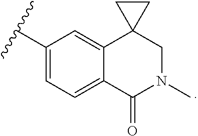 [SMILES: *c1ccc2c(c1)C1(CC1)CN(C)C2=O]

In some embodiments, Cy^{A} is

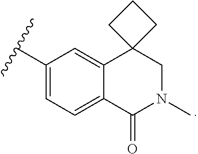 [SMILES: *c1ccc2c(c1)C1(CCC1)CN(C)C2=O]

In some embodiments, Cy^{A} is

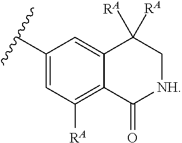

In some embodiments, Cy^{A} is

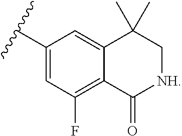 [SMILES: *c1cc(F)c2c(c1)C(C)(C)CNC2=O]

In some embodiments, Cy^{A} is

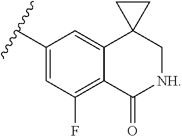 [SMILES: *c1cc(F)c2c(c1)C1(CC1)CNC2=O]

In some embodiments, Cy^{A} is

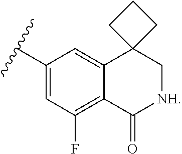 [SMILES: *c1cc(F)c2c(c1)C1(CCC1)CNC2=O]

In some embodiments, Cy^{A} is

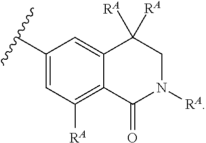

In some embodiments, Cy^{A} is

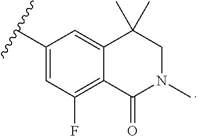 [SMILES: *c1cc(F)c2c(c1)C(C)(C)CN(C)C2=O]

In some embodiments, Cy^{A} is

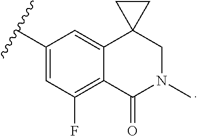 [SMILES: *c1cc(F)c2c(c1)C1(CC1)CN(C)C2=O]

In some embodiments, Cy^{A} is

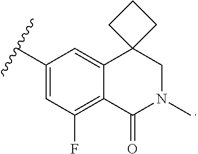 [SMILES: *c1cc(F)c2c(c1)C1(CCC1)CN(C)C2=O]

In some embodiments, Cy^{A} is

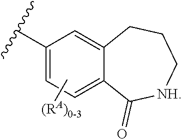

In some embodiments, Cy^{A} is

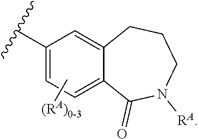

In some embodiments, Cy^{A} is

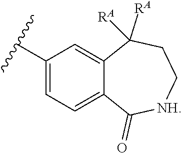

In some embodiments, Cy^{A} is

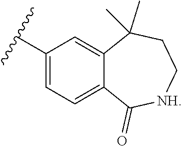 [SMILES: *c1ccc2c(c1)C(C)(C)CCNC2=O]

In some embodiments, Cy^{A} is

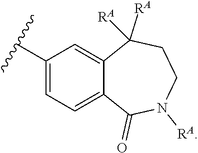

In some embodiments, Cy^{A} is

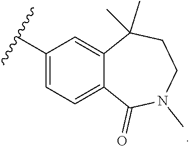 [SMILES: *c1ccc2c(c1)C(C)(C)CCN(C)C2=O]

In some embodiments, Cy^{A} is

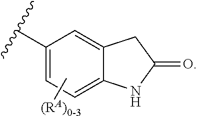

In some embodiments, Cy^{A} is

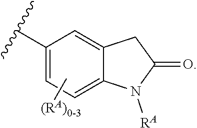

In some embodiments, Cy^{A} is

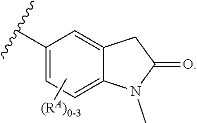

In some embodiments, Cy^{A} is

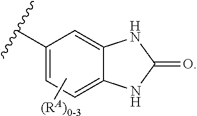

In some embodiments, Cy^{A} is

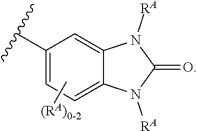

In some embodiments, Cy^{A} is

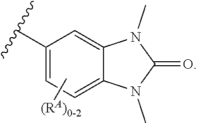

In some embodiments, Cy^{A} is

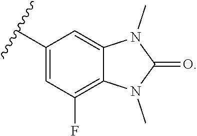 [SMILES: *c1cc(F)c2c(c1)n(C)c(=O)n2C]

In some embodiments, Cy^{A} is

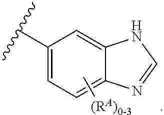

In some embodiments, Cy^{A} is

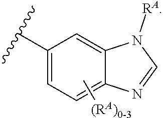

In some embodiments, Cy^{A} is

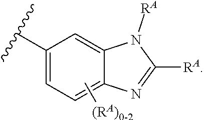

In some embodiments, Cy^{A} is

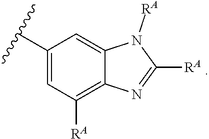

In some embodiments, Cy^{A} is

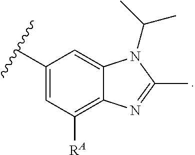

In some embodiments, Cy^{A} is

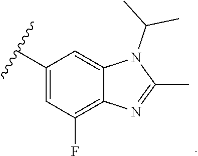 [SMILES: *c1cc(F)c2nc(C)n(C(C)C)c2c1]

In some embodiments, Cy^{A} is

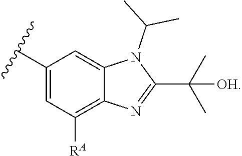

In some embodiments, Cy^{A} is

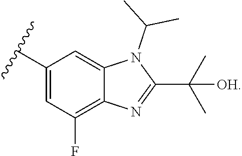 [SMILES: *c1cc(F)c2nc(C(C)(C)O)n(C(C)C)c2c1]

In some embodiments, Cy^{A} is

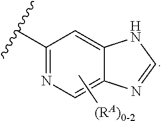

In some embodiments, Cy^{A} is

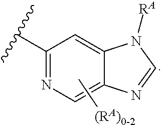

In some embodiments, Cy^{A} is

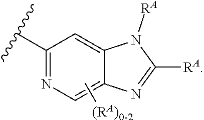

In some embodiments, Cy^{A} is

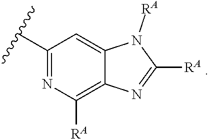

In some embodiments, Cy^{A} is

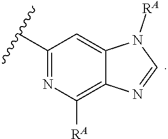

In some embodiments, Cy^{A} is

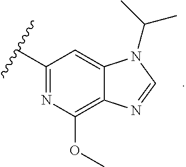 [SMILES: *c1cc2c(ncn2C(C)C)c(OC)n1]

In some embodiments, Cy^{A} is a 3-8 membered saturated or partially unsaturated monocyclic heterocyclic ring having 1-2 heteroatoms independently selected from nitrogen, oxygen, phosphorus, and sulfur. In some embodiments, Cy^{A} is a 3-membered saturated or partially unsaturated monocyclic heterocyclic ring having 1-2 heteroatoms independently selected from nitrogen, oxygen, phosphorus, and sulfur. In some embodiments, Cy^{A} is a 4-membered saturated or partially unsaturated monocyclic heterocyclic ring having 1-2 heteroatoms independently selected from nitrogen, oxygen, phosphorus, and sulfur. In some embodiments, Cy^{A} is a 5-membered saturated or partially unsaturated monocyclic heterocyclic ring having 1-2 heteroatoms independently selected from nitrogen, oxygen, phosphorus, and sulfur. In some embodiments, Cy^{A} is a 6-membered saturated or partially unsaturated monocyclic heterocyclic ring having 1-2 heteroatoms independently selected from nitrogen, oxygen, phosphorus, and sulfur. In some embodiments, Cy^{A} is a 7-membered saturated or partially unsaturated monocyclic heterocyclic ring having 1-2 heteroatoms independently selected from nitrogen, oxygen, phosphorus, and sulfur. In some embodiments, Cy^{A} is an 8-membered saturated or partially unsaturated monocyclic heterocyclic ring having 1-2 heteroatoms independently selected from nitrogen, oxygen, phosphorus, and sulfur.

In some embodiments, Cy^{A} is

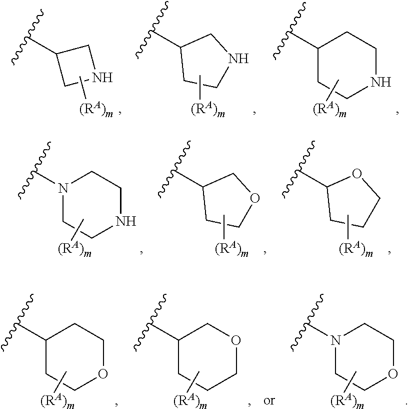

In some embodiments, Cy^{A} is

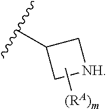

In some embodiments, Cy^{A} is

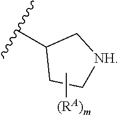

In some embodiments, Cy^{A} is

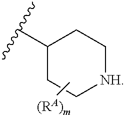

In some embodiments, Cy^{A} is

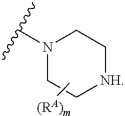

In some embodiments, Cy^{A} is

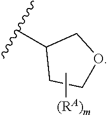

In some embodiments, Cy^{A} is

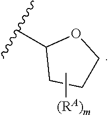

In some embodiments, Cy^{A} is

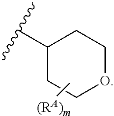

In some embodiments, Cy^{A} is

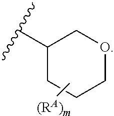

In some embodiments, Cy^{A} is

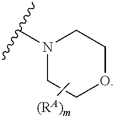

In some embodiments, Cy^{A} is

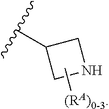

In some embodiments, Cy^{A} is

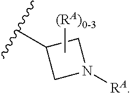

In some embodiments, Cy^{A} is

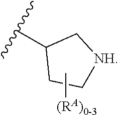

In some embodiments, Cy^{A} is

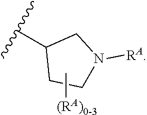

In some embodiments, Cy^{A} is

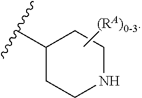

In some embodiments, Cy^{A} is

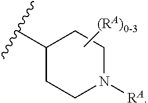

In some embodiments, Cy^{A} is

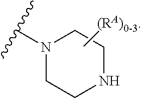

In some embodiments, Cy^{A} is

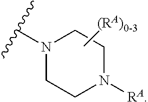

In some embodiments, Cy^{A} is a 7-12 membered saturated or partially unsaturated bicyclic heterocyclic ring having 1-4 heteroatoms independently selected from nitrogen, oxygen, phosphorus, and sulfur. In some embodiments, Cy^{A} is a 7-membered saturated or partially unsaturated bicyclic heterocyclic ring having 1-4 heteroatoms independently selected from nitrogen, oxygen, phosphorus, and sulfur. In some embodiments, Cy^{A} is an 8-membered saturated or partially unsaturated bicyclic heterocyclic ring having 1-4 heteroatoms independently selected from nitrogen, oxygen, phosphorus, and sulfur. In some embodiments, Cy^{A} is a 9-membered saturated or partially unsaturated bicyclic heterocyclic ring having 1-4 heteroatoms independently selected from nitrogen, oxygen, phosphorus, and sulfur. In some embodiments, Cy^{A} is a 10-membered saturated or partially unsaturated bicyclic heterocyclic ring having 1-4 heteroatoms independently selected from nitrogen, oxygen, phosphorus, and sulfur. In some embodiments, Cy^{A} is an 11-membered saturated or partially unsaturated bicyclic heterocyclic ring having 1-4 heteroatoms independently selected from nitrogen, oxygen, phosphorus, and sulfur.

In some embodiments, Cy^{A} is

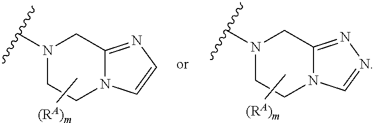

In some embodiments, Cy^{A} is

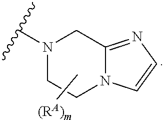

In some embodiments, Cy^{A} is

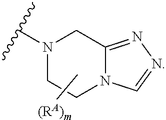

In some embodiments, Cy^{A} is a 10-14 membered tricyclic heteroaryl ring having 1-6 heteroatoms independently selected from nitrogen, oxygen, and sulfur. In some embodiments, Cy^{A} is a 10-14 membered aromatic or partially unsaturated tricyclic heteroaryl ring having 1-6 heteroatoms independently selected from nitrogen, oxygen, and sulfur. In some embodiments, Cy^{A} is a 10-14 membered partially unsaturated tricyclic heteroaryl ring having 1-6 heteroatoms independently selected from nitrogen, oxygen, and sulfur.

In some embodiments, Cy^{A} is a 10-membered tricyclic heteroaryl ring having 1-6 heteroatoms independently selected from nitrogen, oxygen, and sulfur. In some embodiments, Cy^{A} is an 11-membered tricyclic heteroaryl ring having 1-6 heteroatoms independently selected from nitrogen, oxygen, and sulfur. In some embodiments, Cy^{A} is a 12-membered tricyclic heteroaryl ring having 1-6 heteroatoms independently selected from nitrogen, oxygen, and sulfur. In some embodiments, Cy^{A} is a 13-membered tricyclic heteroaryl ring having 1-6 heteroatoms independently selected from nitrogen, oxygen, and sulfur. In some embodiments, Cy^{A} is a 14-membered tricyclic heteroaryl ring having 1-6 heteroatoms independently selected from nitrogen, oxygen, and sulfur.

In some embodiments, Cy^{A} is

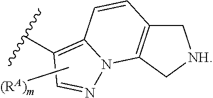

In some embodiments, Cy^{A} is

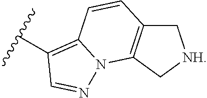 [SMILES: *c1cnn2c3c(ccc12)CNC3]

In some embodiments, Cy^{A} is

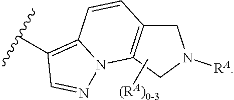

In some embodiments, Cy^{A} is

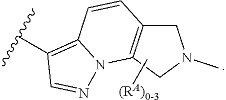

In some embodiments, Cy^{A} is

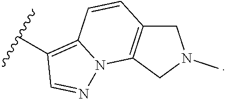 [SMILES: *c1cnn2c3c(ccc12)CN(C)C3]

In some embodiments, Cy^{A} is

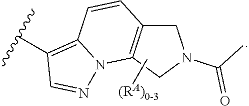

In some embodiments, Cy^{A} is

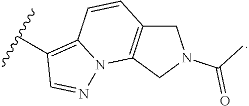 [SMILES: *c1cnn2c3c(ccc12)CN(C(C)=O)C3]

In some embodiments, Cy^{A} is

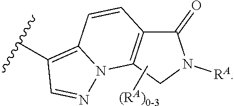

In some embodiments, Cy^{A} is

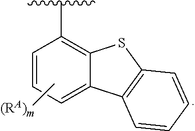

In some embodiments, Cy^{A} is

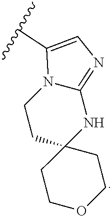

In some embodiments, Cy^{A} is

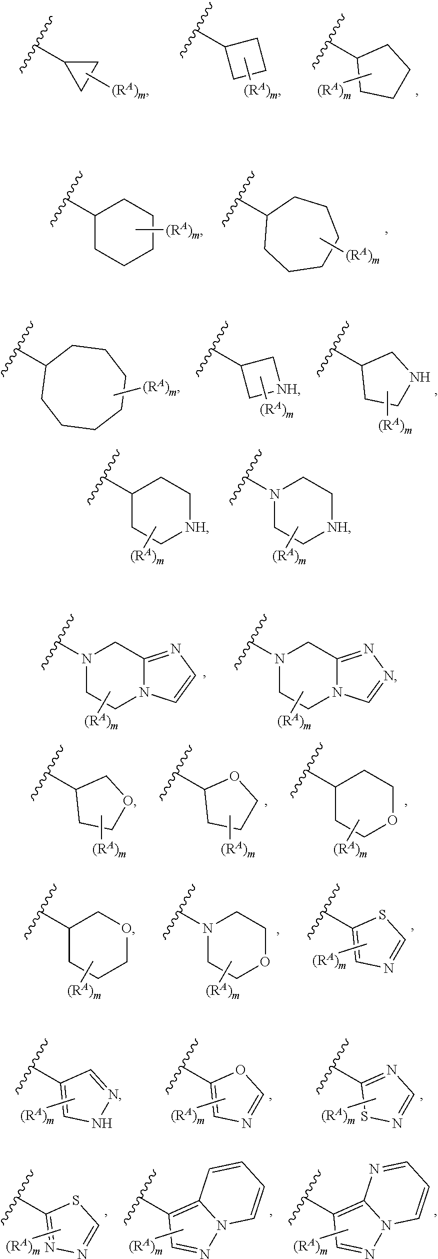

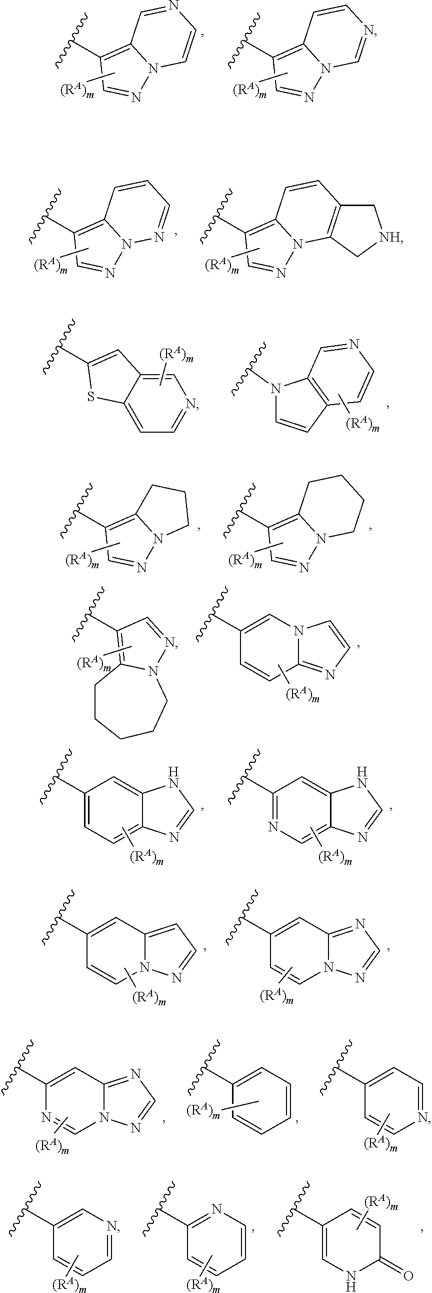

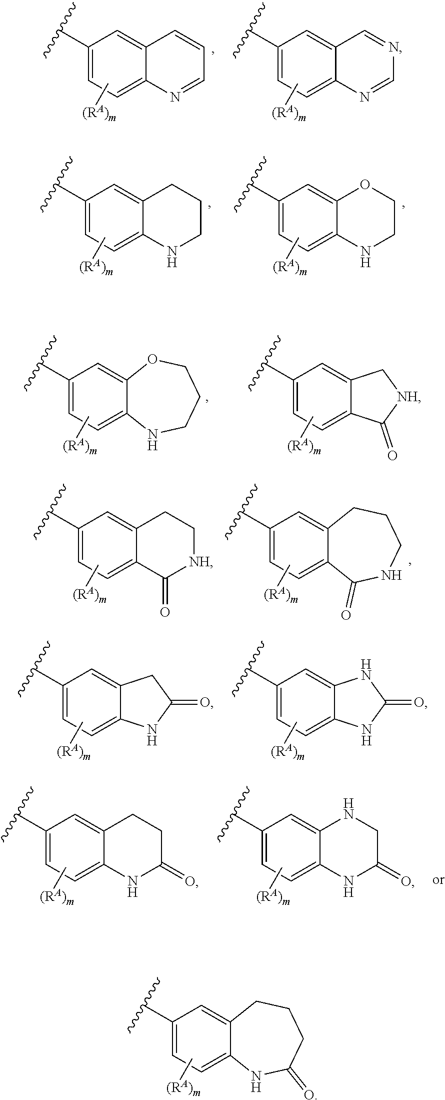

In some embodiments, Cy^{A} is selected from the groups depicted in the compounds in Table 1.

In some embodiments, two instances of R^{B} are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³.

In some embodiments, two instances of R^{B} are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³.

In some embodiments, two instances of R^{X} are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³.

In some embodiments, two instances of X⁰ are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³.

In some embodiments, an instance of X⁰ and an instance of R^{A} are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³.

In some embodiments, an instance of R^{A} and an instance of R^{X} are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³.

In some embodiments, L is a covalent bond or a bivalent C₁₋₃ aliphatic group, wherein one carbon is optionally replaced by —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, —S(O)₂—, C₃₋₆ cycloalkylene, or 3-6 membered heterocycloalkylene having 1-2 heteroatoms independently selected from nitrogen, oxygen, and sulfur; wherein each of said C₃₋₆ cycloalkylene and 3-6 membered heterocycloalkylene is optionally substituted with one instance of R¹ or C₁₋₆ aliphatic.

In some embodiments, L is a covalent bond. In some embodiments, L is ethynylene. In some embodiments, L is C₁₋₃ aliphatic, wherein one carbon is optionally replaced by —C(O)—. In some embodiments, L is C₁₋₃ aliphatic, wherein one carbon is optionally replaced by —O—. In some embodiments, L is C₁₋₃ aliphatic, wherein one carbon is optionally replaced by —NH—. In some embodiments, L is C₁₋₃ aliphatic. In some embodiments, L is CH₂. In some embodiments, L is —NH—. In some embodiments, L is —N(R^{L})—. In some embodiments, L is —NHC(O)—. In some embodiments, L is —N(R^{L})C(O)—. In some embodiments, L is —C(O)NH—. In some embodiments, L is —C(O)N(R^{L})—. In some embodiments, L is —NHS(O)₂—. In some embodiments, L is —N(R^{L})S(O)₂—. In some embodiments, L is —S(O)₂NH—. In some embodiments, L is —S(O)₂N(R^{L})—. In some embodiments, L is —O—. In some embodiments, L is —C(O)—. In some embodiments, L is —OC(O)—. In some embodiments, L is —C(O)O—. In some embodiments, L is —S—. In some embodiments, L is —S(O)—. In some embodiments, L is —S(O)₂—. In some embodiments, L is C₃₋₆ cycloalkylene that is optionally substituted with one instance of R¹ or C₁₋₆ aliphatic. In some embodiments, L is a 3-6 membered heterocycloalkylene having 1-2 heteroatoms independently selected from nitrogen, oxygen, and sulfur that is optionally substituted with one instance of R¹ or C₁₋₆ aliphatic.

In some embodiments, Cy^{B} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heterocyclic ring having 1-6 heteroatoms independently selected from nitrogen, oxygen, and sulfur; wherein Cy^{B} is substituted with n instances of R^{B}.

In some embodiments, Cy^{B} is phenyl substituted with n instances of R^{B}.

In some embodiments, Cy^{B} is a 5-6 membered monocyclic heteroaryl ring having 1-4 heteroatoms independently selected from nitrogen, oxygen, and sulfur, wherein Cy^{B} is substituted with n instances of R^{B}.

In some embodiments, Cy^{B} is a 5-membered monocyclic heteroaryl ring having 1-4 heteroatoms independently selected from nitrogen, oxygen, and sulfur, wherein Cy^{B} is substituted with n instances of R^{B}. In some embodiments, Cy^{B} is

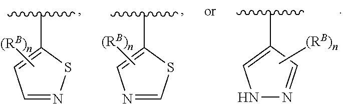

In some embodiments, Cy is

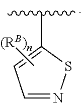

In some embodiments, Cy^{B} is

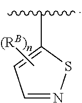

In some embodiments, Cy^{B} is

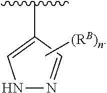

In some embodiments, Cy^{B} is a 6-membered monocyclic heteroaryl ring having 1-4 heteroatoms independently selected from nitrogen, oxygen, and sulfur, wherein Cy^{B} is substituted with n instances of R^{B}. In some embodiments, Cy^{B} is

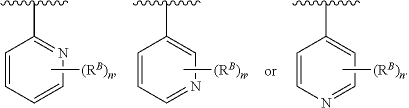

In some embodiments, Cy^{B} is

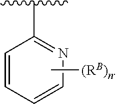

In some embodiments, Cy^{B} is

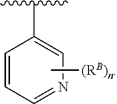

In some embodiments, Cy^{B} is

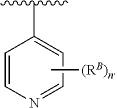

In some embodiments, Cy^{B} is

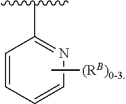

In some embodiments, Cy^{B} is

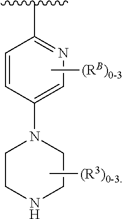

In some embodiments, Cy^{B} is

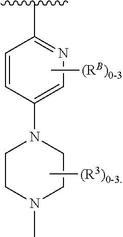

In some embodiments, Cy^{B} is

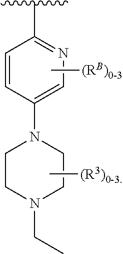

In some embodiments, Cy^{B} is

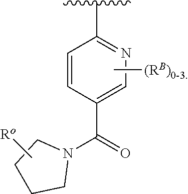

In some embodiments, Cy^{B} is

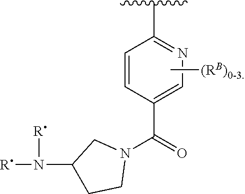

In some embodiments, Cy^{B} is

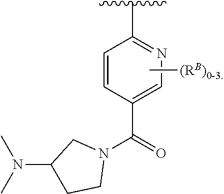

In some embodiments, Cy^{B} is

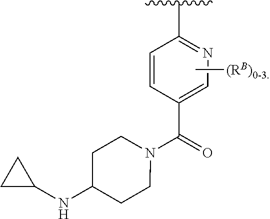

In some embodiments, Cy^{B} is

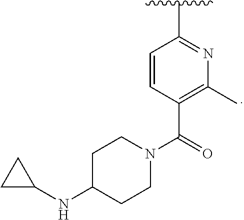 [SMILES: *c1ccc(C(=O)N2CCC(NC3CC3)CC2)c(C)n1]

In some embodiments, Cy^{B} is

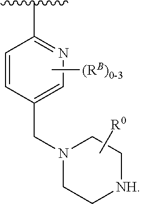

In some embodiments, Cy^{B} is

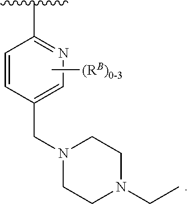

In some embodiments, Cy^{B} is an 8-10 membered bicyclic heteroaryl ring having 1-4 heteroatoms independently selected from nitrogen, oxygen, and sulfur, wherein Cy^{B} is substituted with n instances of R^{B}.

In some embodiments, Cy^{B} is

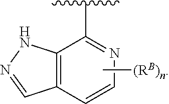

In some embodiments, Cy^{B} is a 3-8 membered saturated carbocyclic ring, wherein Cy^{B} is substituted with n instances of R^{B}. In some embodiments, Cy^{B} is a 3-7 membered saturated carbocyclic ring, wherein Cy^{B} is substituted with n instances of R^{B}. In some embodiments, Cy^{B} is a 4-7 membered saturated carbocyclic ring, wherein Cy^{B} is substituted with n instances of R^{B}. In some embodiments, Cy^{B} is a 5-7 membered saturated carbocyclic ring, wherein Cy^{B} is substituted with n instances of R^{B}. In some embodiments, Cy^{B} is a 5-6 membered saturated carbocyclic ring, wherein Cy^{B} is substituted with n instances of R^{B}. In some embodiments, Cy^{B} is a 4-6 membered saturated carbocyclic ring, wherein Cy^{B} is substituted with n instances of R^{B}.

In some embodiments, Cy^{B} is a cyclopropyl ring. In some embodiments, Cy^{B} is a cyclobutyl ring. In some embodiments, Cy^{B} is a cyclopentyl ring. In some embodiments, Cy^{B} is a cyclohexyl ring. In some embodiments, Cy^{B} is a cycloheptyl ring. In some embodiments, Cy^{B} is a cyclooctyl ring.

In some embodiments, Cy^{B} is

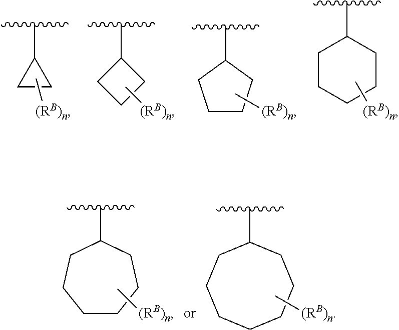

In some embodiments, Cy^{B} is

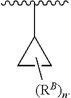

In some embodiments, Cy^{B} is

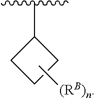

In some embodiments, Cy^{B} is

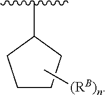

In some embodiments, Cy^{B} is

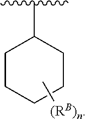

In some embodiments, Cy^{B} is

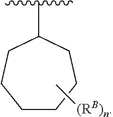

In some embodiments, Cy^{B} is

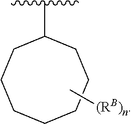

In some embodiments, Cy^{B} is

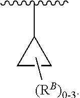

In some embodiments, Cy^{B} is

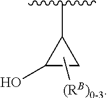

In some embodiments, Cy^{B} is

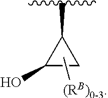

In some embodiments, Cy^{B} is

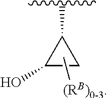

In some embodiments, Cy^{B}

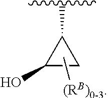

In some embodiments, Cy^{B} is

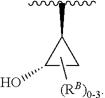

In some embodiments, Cy^{B} is

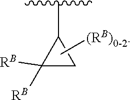

In some embodiments, Cy^{B} is

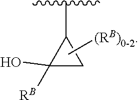

In some embodiments, Cy^{B} is

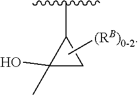

In some embodiments, Cy^{B} is

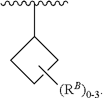

In some embodiments, Cy^{B} is

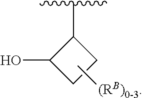

In some embodiments, Cy^{B} is

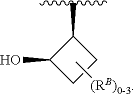

In some embodiments, Cy^{B} is

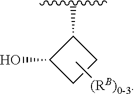

In some embodiments, Cy^{B} is

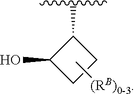

In some embodiments, Cy^{B} is

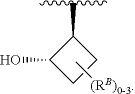

In some embodiments, Cy^{B} is

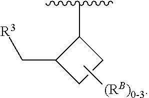

In some embodiments, Cy^{B} is

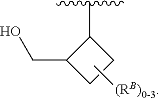

In some embodiments, Cy^{B} is

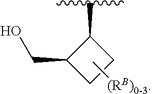

In some embodiments, Cy^{B} is

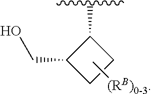

In some embodiments, Cy^{B} is

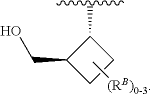

In some embodiments, Cy^{B} is

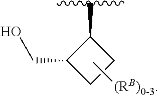

In some embodiments, Cy^{B} is

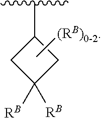

In some embodiments, Cy^{B} is

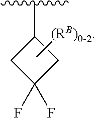

In some embodiments, Cy^{B} is

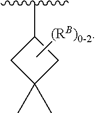

In some embodiments, Cy^{B} is

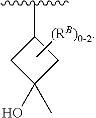

In some embodiments, Cy^{B} is

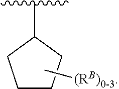

In some embodiments, Cy^{B} is

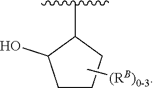

In some embodiments, Cy^{B} is

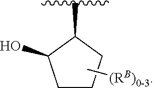

In some embodiments, Cy^{B} is

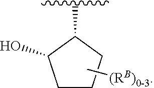

In some embodiments, Cy^{B} is

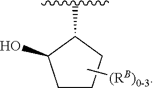

In some embodiments, Cy^{B} is

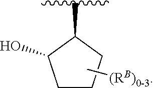

In some embodiments, Cy^{B} is

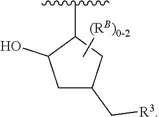

In some embodiments, Cy^{B} is

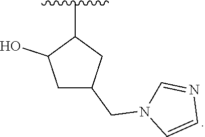 [SMILES: *C1CC(Cn2ccnc2)CC1O]

In some embodiments, Cy^{B} is

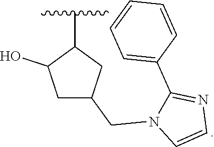 [SMILES: *C1CC(Cn2ccnc2-c2ccccc2)CC1O]

In some embodiments, Cy^{B} is

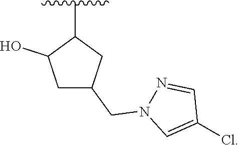 [SMILES: *C1CC(Cn2cc(Cl)cn2)CC1O]

In some embodiments, Cy^{B} is

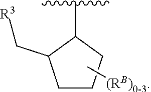

In some embodiments, Cy^{B} is

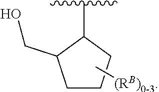

In some embodiments, Cy^{B} is

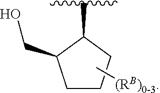

In some embodiments, Cy^{B} is

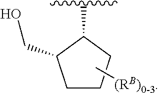

In some embodiments, Cy^{B} is

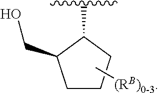

In some embodiments, Cy^{B} is

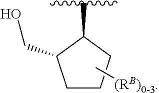

In some embodiments, Cy^{B} is

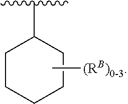

In some embodiments, Cy^{B} is

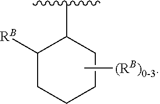

In some embodiments, Cy^{B} is

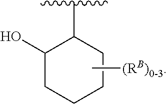

In some embodiments, Cy^{B} is

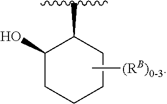

In some embodiments, Cy^{B} is

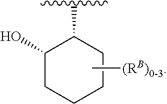

In some embodiments, Cy^{B} is

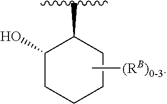

In some embodiments, Cy^{B} is

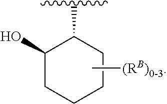

In some embodiments, Cy^{B} is

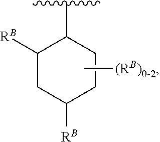

In some embodiments, Cy^{B} is

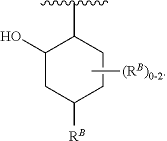

In some embodiments, Cy^{B} is

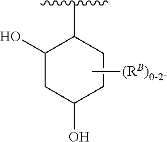

In some embodiments, Cy^{B} is

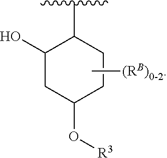

In some embodiments, Cy^{B} is

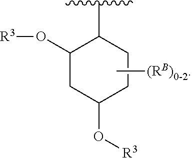

In some embodiments, Cy^{B} is

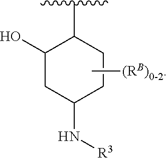

In some embodiments, Cy^{B} is

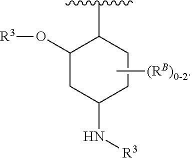

In some embodiments, Cy^{B} is

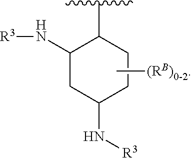

In some embodiments, Cy^{B} is

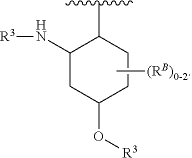

In some embodiments, Cy^{B} is

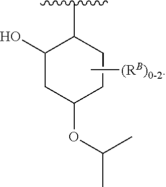

In some embodiments, Cy^{B} is

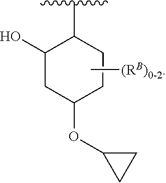

In some embodiments, Cy^{B} is

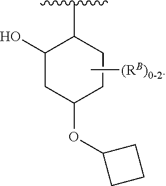

In some embodiments, Cy^{B} is

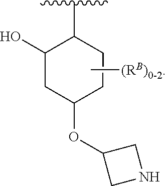

In some embodiments, Cy^{B} is

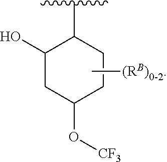

In some embodiments, Cy^{B} is

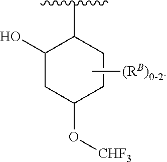

In some embodiments, Cy^{B} is

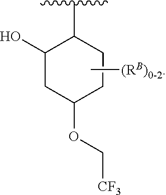

In some embodiments, Cy^{B} is

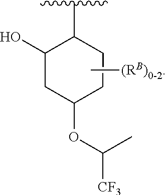

In some embodiments, Cy^{B} is

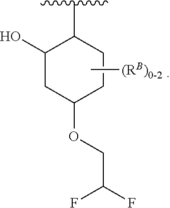

In some embodiments, Cy^{B} is

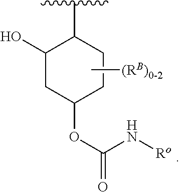

In some embodiments, Cy^{B} is

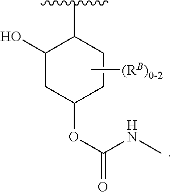

In some embodiments, Cy^{B} is

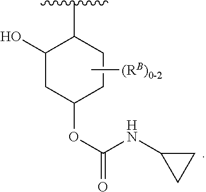

In some embodiments, Cy^{B} is

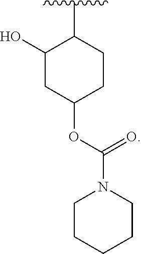 [SMILES: *C1CCC(OC(=O)N2CCCCC2)CC1O]

In some embodiments, Cy^{B} is

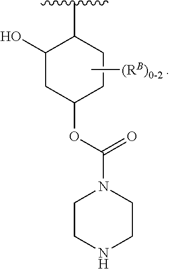

In some embodiments, Cy^{B} is

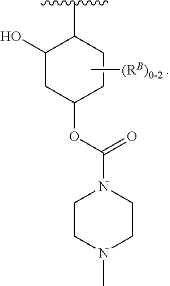

In some embodiments, Cy^{B} is

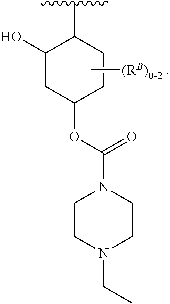

In some embodiments, Cy^{B} is

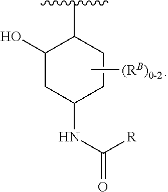

In some embodiments, Cy^{B} is

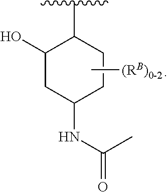

In some embodiments, Cy^{B} is

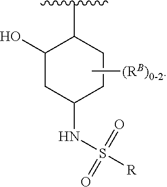

In some embodiments, Cy^{B} is

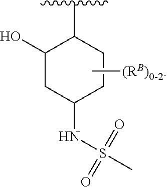

In some embodiments, Cy^{B} is

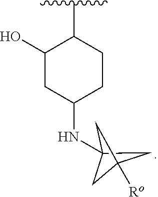

In some embodiments, Cy^{B} is

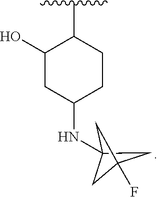 [SMILES: *C1CCC(NC23CC(F)(C2)C3)CC1O]

In some embodiments, Cy^{B} is

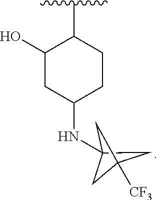 [SMILES: *C1CCC(NC23CC(C(F)(F)F)(C2)C3)CC1O]

In some embodiments, Cy^{B} is

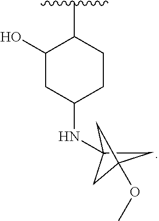 [SMILES: *C1CCC(NC23CC(OC)(C2)C3)CC1O]

In some embodiments, Cy^{B} is

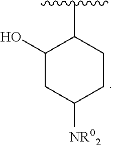

In some embodiments, Cy^{B} is

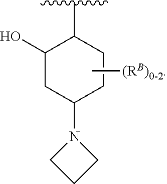

In some embodiments, Cy^{B} is

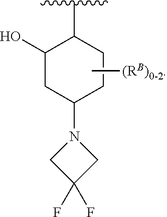

In some embodiments, Cy^{B} is

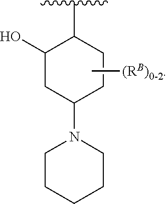

In some embodiments, Cy^{B} is

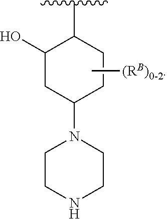

In some embodiments, Cy^{B} is

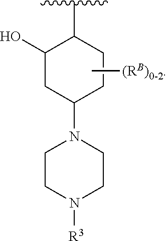

In some embodiments, Cy^{B} is

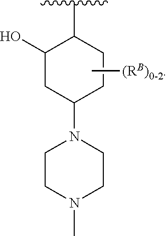

In some embodiments, Cy^{B} is

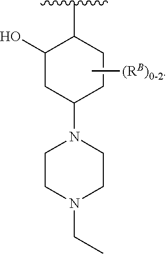

In some embodiments, Cy^{B} is

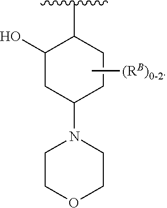

In some embodiments, Cy^{B} is

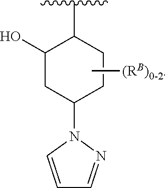

In some embodiments, Cy^{B} is

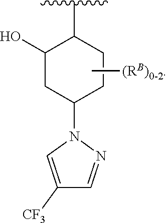

In some embodiments, Cy^{B} is

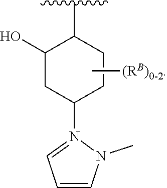

In some embodiments, Cy^{B} is

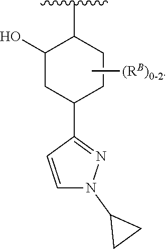

In some embodiments, Cy^{B} is

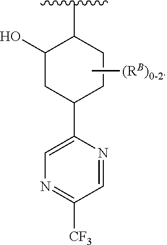

In some embodiments, Cy^{B} is

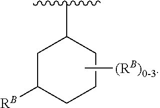

In some embodiments, Cy^{B} is

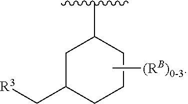

In some embodiments, Cy^{B} is

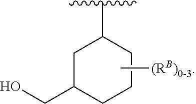

In some embodiments, Cy^{B} is

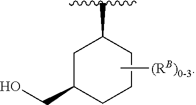

In some embodiments, Cy^{B} is

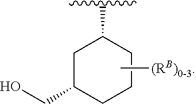

In some embodiments, Cy^{B} is

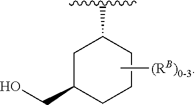

In some embodiments, Cy^{B} is

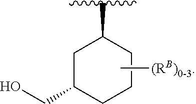

In some embodiments, Cy^{B} is

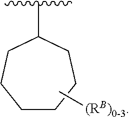

In some embodiments, Cy^{B} is

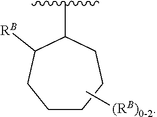

In some embodiments, Cy^{B} is

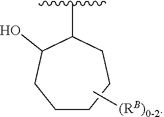

In some embodiments, Cy^{B} is

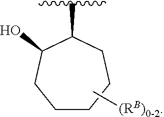

In some embodiments, Cy^{B} is

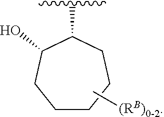

In some embodiments, Cy^{B} is

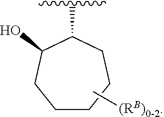

In some embodiments, Cy^{B} is

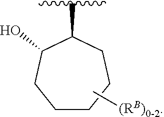

In some embodiments, Cy^{B} is

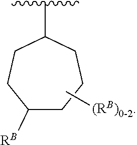

In some embodiments, Cy^{B} is

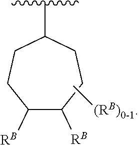

In some embodiments, Cy^{B} is

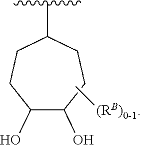

In some embodiments, Cy^{B} is

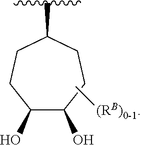

In some embodiments, Cy^{B} is

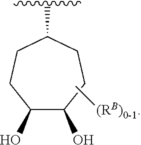

In some embodiments, Cy^{B} is

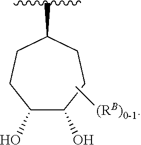

In some embodiments, Cy^{B} is

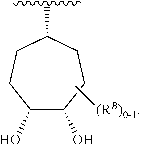

In some embodiments, Cy^{B} is

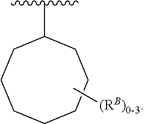

In some embodiments, Cy^{B} is

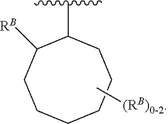

In some embodiments, Cy^{B} is

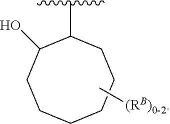

In some embodiments, Cy^{B} is

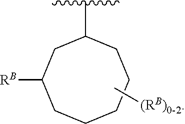

In some embodiments, Cy^{B} is

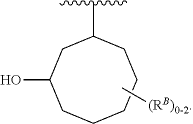

In some embodiments, Cy^{B} is

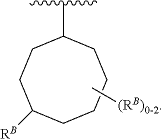

In some embodiments, Cy^{B} is

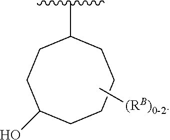

In some embodiments, Cy^{B} is

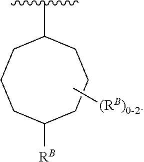

In some embodiments, Cy^{B} is

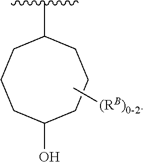

In some embodiments, Cy^{B} is

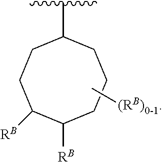

In some embodiments, Cy^{B} is

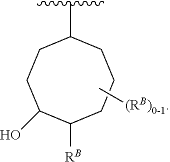

In some embodiments, Cy^{B} is a 3-8 membered saturated or partially unsaturated carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³. In some embodiments, Cy^{B} is a 3-7 membered saturated or partially unsaturated carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³. In some embodiments, Cy^{B} is a 4-7 membered saturated or partially unsaturated carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³. In some embodiments, Cy^{B} is a 5-7 membered saturated or partially unsaturated carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R^{B}. In some embodiments, Cy^{B} is a 5-6 membered saturated or partially unsaturated carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R^{B}. In some embodiments, Cy^{B} is a 4-6 membered saturated or partially unsaturated carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³.

In some embodiments, Cy^{B} is a 3-membered saturated or partially unsaturated monocyclic carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³.

In some embodiments, Cy^{B} is

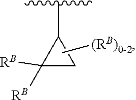

wherein two instances of R^{B} are taken together with their intervening atoms to form a 4-8 membered saturated or partially unsaturated ring having 0-4 heteroatoms independently selected from nitrogen, oxygen, phosphorus, and sulfur; wherein said ring is substituted with r instances of R³.

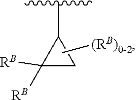

In some embodiments, Cy^{B} is R^{B}, wherein two instances of R^{B} are taken together with their intervening atoms to form a 4-8 membered saturated ring, wherein said ring is substituted with r instances of R³. In some embodiments, Cy^{B} is

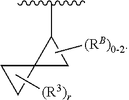

In some embodiments, Cy^{B} is

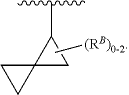

In some embodiments, Cy^{B} is

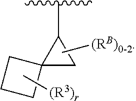

In some embodiments, Cy^{B} is

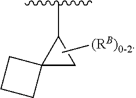

In some embodiments, Cy^{B} is

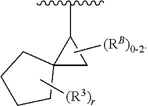

In some embodiments, Cy^{B} is

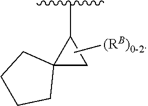

In some embodiments, Cy^{B} is a 4-membered saturated or partially unsaturated monocyclic carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³.

In some embodiments, Cy^{B} is

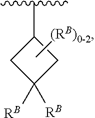

wherein two instances of R^{B} are taken together with their intervening atoms to form a 4-8 membered saturated or partially unsaturated ring having 0-4 heteroatoms independently selected from nitrogen, oxygen, phosphorus, and sulfur; wherein said ring is substituted with r instances of R³.

In some embodiments, Cy^{B} is

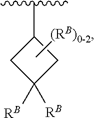

wherein two instances of R^{B} are taken together with their intervening atoms to form a 4-8 membered saturated ring, wherein said ring is substituted with r instances of R³. In some embodiments, Cy^{B} is

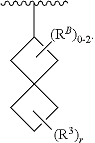

In some embodiments, Cy^{B} is

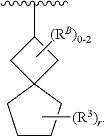

In some embodiments, Cy^{B} is

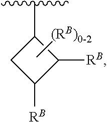

In some embodiments, Cy^{B} is

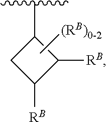

wherein two instances of R^{B} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³.

In some embodiments, Cy^{B} is

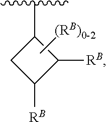

wherein two instances of R^{B} are taken together with their intervening atoms to form a 4-8 membered saturated ring, wherein said ring is substituted with r instances of R³. In some embodiments, Cy^{B} is

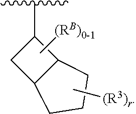

In some embodiments, Cy^{B} is

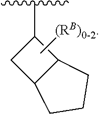

In some embodiments, Cy^{B} is

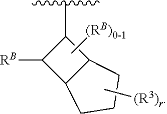

In some embodiments, Cy^{B} is

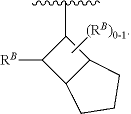

In some embodiments, Cy^{B} is

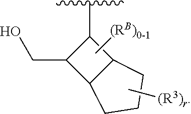

In some embodiments, Cy^{B} is

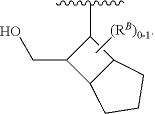

In some embodiments, Cy^{B} is a 5-membered saturated or partially unsaturated monocyclic carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³.

In some embodiments, Cy^{B} is

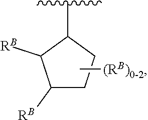

wherein two instances of R^{B} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³.

In some embodiments, Cy^{B} is

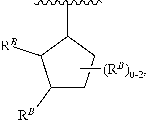

wherein two instances of R^{B} are taken together with their intervening atoms to form a 5-6 membered aromatic ring having 0-4 heteroatoms independently selected from nitrogen, oxygen, and sulfur; wherein said ring is substituted with r instances of R³. In some embodiments, Cy^{B} is

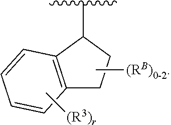

In some embodiments, Cy^{B} is

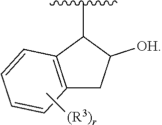

In some embodiments, Cy^{B} is

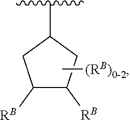

wherein two instances of R^{B} are taken together with their intervening atoms to form a 5-6 membered aromatic ring having 0-4 heteroatoms independently selected from nitrogen, oxygen, and sulfur; wherein said ring is substituted with r instances of R³. In some embodiments, Cy^{B} is

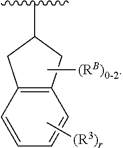

In some embodiments, Cy^{B} is

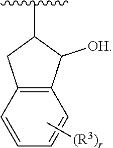

In some embodiments, Cy^{B} is a 6-membered saturated or partially unsaturated monocyclic carbocyclic ring, wherein Cy^{B} is substituted with at least 2 instances of R^{B}, wherein said two instances of R^{B} are taken together with their intervening atoms to form a 4-8 membered saturated, partially unsaturated or aromatic ring having 0-4 heteroatoms independently selected from nitrogen, oxygen, phosphorus, and sulfur; wherein said ring is substituted with r instances of R³.

In some embodiments, Cy^{B} is

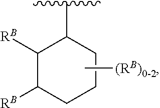

wherein two instances of R^{B} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³.

In some embodiments, Cy^{B} is

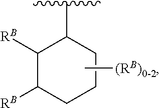

wherein two instances of R^{B} are taken together with their intervening atoms to form a 5-6 membered aromatic ring, wherein said ring is substituted with r instances of R³. In some embodiments, Cy^{B} is

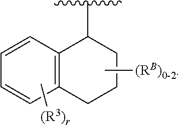

In some embodiments, Cy^{B} is

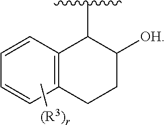

In some embodiments, Cy^{B} is

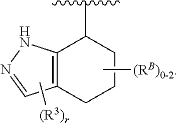

In some embodiments, Cy^{B} is a 5-8 membered saturated or partially unsaturated bridged bicyclic or fused carbocyclic ring. In some embodiments, Cy^{B} is a 5-8 membered saturated bridged bicyclic or fused carbocyclic ring. In some embodiments, Cy^{B} is a 6-7 membered saturated bridged bicyclic or fused carbocyclic ring. In some embodiments, Cy^{B} is a 7-8 membered saturated bridged bicyclic or fused carbocyclic ring.

In some embodiments, Cy^{B} is a 5-8 membered saturated or partially unsaturated fused carbocyclic ring. In some embodiments, Cy^{B} is

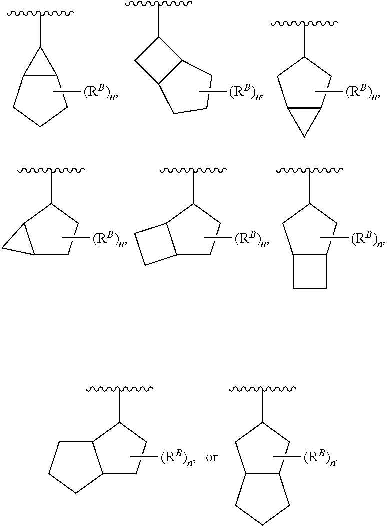

In some embodiments, Cy^{B} is

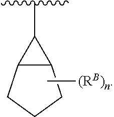

In some embodiments, Cy^{B} is

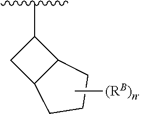

In some embodiments, Cy^{B} is

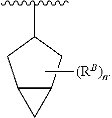

In some embodiments, Cy^{B} is

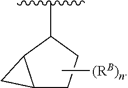

In some embodiments, Cy^{B} is

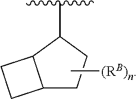

In some embodiments, Cy^{B} is

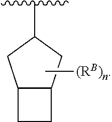

In some embodiments, Cy^{B} is

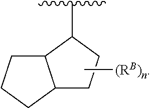

In some embodiments, Cy^{B} is

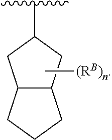

In some embodiments, Cy^{B} is a 5-8 membered saturated or partially unsaturated bridged carbocyclic ring. In some embodiments, Cy^{B} is

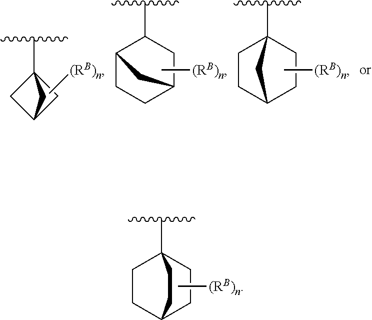

In some embodiments, Cy^{B} is

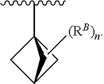

In some embodiments, Cy^{B} is

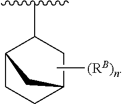

In some embodiments, Cy^{B} is

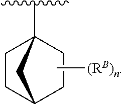

In some embodiments, Cy^{B} is

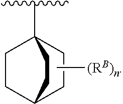

In some embodiments, Cy^{B} is a 5-8 membered saturated or partially unsaturated spirofused carbocyclic ring. In some embodiments, Cy^{B} is a 5-8 membered saturated spirofused carbocyclic ring. In some embodiments, Cy^{B} is a 6-7 membered saturated spirofused carbocyclic ring. In some embodiments, Cy^{B} is a 7-8 membered saturated spirofused carbocyclic ring. In some embodiments, Cy^{B} is

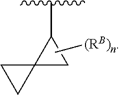

In some embodiments, Cy^{B} is

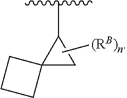

In some embodiments, Cy^{B} is

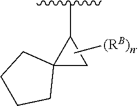

In some embodiments, Cy^{B} is

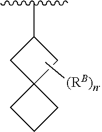

In some embodiments, Cy^{B} is

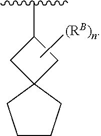

In some embodiments, Cy^{B} is a 3-7 membered partially unsaturated monocyclic carbocyclic ring. In some embodiments, Cy^{B} is a 5-6 membered partially unsaturated monocyclic carbocyclic ring. In some embodiments, Cy^{B} is

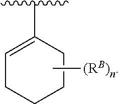

In some embodiments, Cy^{B} is

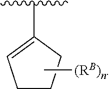

In some embodiments, Cy^{B} is a 3-8 membered saturated or partially unsaturated monocyclic heterocyclic ring having 1-2 heteroatoms independently selected from nitrogen, oxygen, phosphorus, and sulfur. In some embodiments, Cy^{B} is a saturated 3-7 membered monocyclic heterocyclic ring having 1-2 heteroatoms independently selected from nitrogen, oxygen, phosphorus, and sulfur. In some embodiments, Cy^{B} is a saturated 4-7 membered monocyclic heterocyclic ring having 1-2 heteroatoms independently selected from nitrogen, oxygen, phosphorus, and sulfur.

In some embodiments, Cy^{B} is a 4-membered monocyclic heterocyclic ring having 1-2 heteroatoms independently selected from nitrogen, oxygen, phosphorus, and sulfur. In some embodiments, Cy^{B} is 4-membered monocyclic heterocyclic ring having 1-2 heteroatoms independently selected from nitrogen and oxygen. In some embodiments, Cy^{B} is

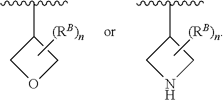

In some embodiments, Cy^{B} is

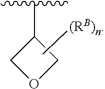

In some embodiments, Cy^{B} is

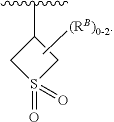

In some embodiments, Cy^{B} is

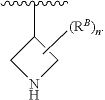

In some embodiments, Cy^{B} is a 5-membered monocyclic heterocyclic ring having 1-2 heteroatoms independently selected from nitrogen, oxygen, phosphorus, and sulfur. In some embodiments, Cy^{B} is

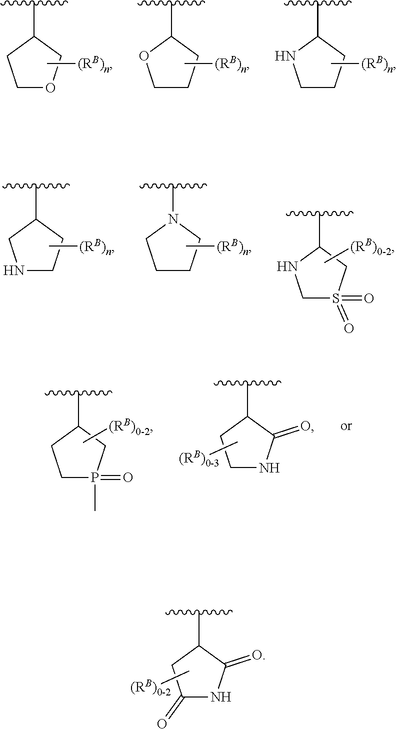

In some embodiments, Cy^{B} is

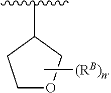

In some embodiments, Cy^{B} is

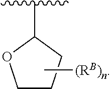

In some embodiments, Cy^{B} is

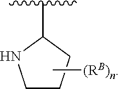

In some embodiments, Cy^{B} is

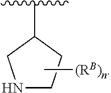

In some embodiments, Cy^{B} is

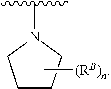

In some embodiments, Cy^{B} is

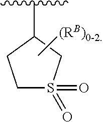

In some embodiments, Cy^{B} is

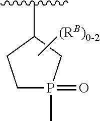

In some embodiments, Cy^{B} is

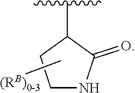

In some embodiments, Cy^{B} is

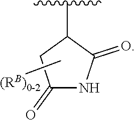

In some embodiments, Cy^{B} is a 6-membered monocyclic heterocyclic ring having 1-2 heteroatoms independently selected from nitrogen, oxygen, phosphorus, and sulfur. In some embodiments, Cy^{B} is a 6-membered monocyclic heterocyclic ring having 1-2 heteroatoms independently selected from nitrogen and oxygen. In some embodiments, Cy^{B} is

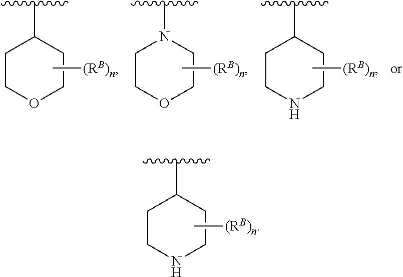

In some embodiments, Cy^{B} is

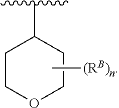

In some embodiments, Cy^{B} is

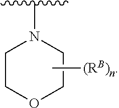

In some embodiments, Cy^{B} is

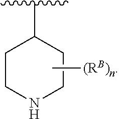

In some embodiments, Cy^{B} is H

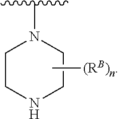

In some embodiments, Cy^{B} is

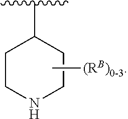

In some embodiments, Cy^{B} is

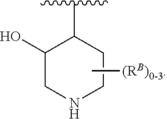

In some embodiments, Cy^{B} is

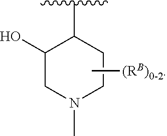

In some embodiments, Cy^{B} is

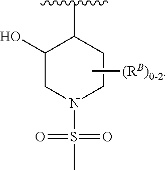

In some embodiments, Cy^{B} is

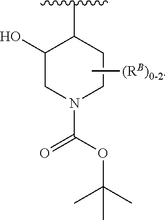

In some embodiments, Cy^{B} is

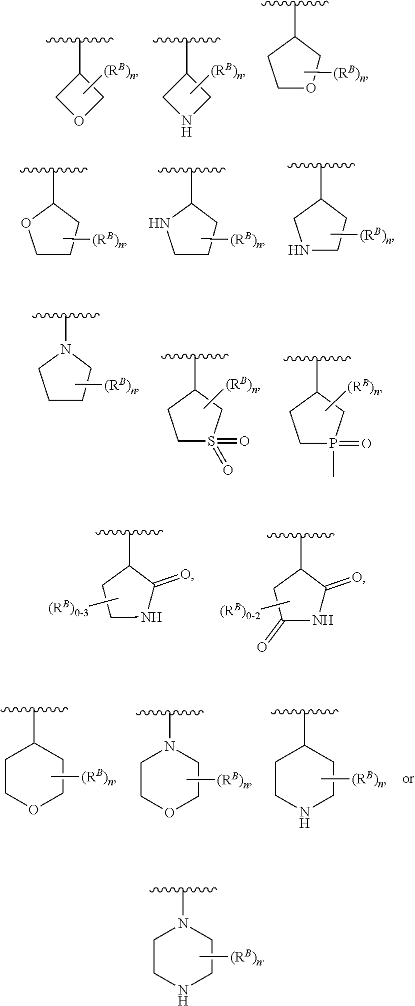

In some embodiments, Cy^{B} is a saturated or partially unsaturated 7-10 membered bicyclic heterocyclic ring having 1-4 heteroatoms independently selected from nitrogen, oxygen, phosphorus, and sulfur, wherein Cy^{B} is substituted with n instances of R^{B}.

In some embodiments, Cy^{B} is a saturated or partially unsaturated 7-10 membered bicyclic heterocyclic ring having 1-2 heteroatoms independently selected from nitrogen, oxygen, and sulfur, wherein Cy^{B} is substituted with n instances of R^{B}. In some embodiments, Cy^{B} is a saturated or partially unsaturated 7-8 membered bicyclic heterocyclic ring having 1-2 heteroatoms independently selected from nitrogen, oxygen, phosphorus, and sulfur, wherein Cy^{B} is substituted with n instances of R^{B}.

In some embodiments, Cy^{B} is a saturated 7-10 membered bridged bicyclic or fused heterocyclic ring having 1-4 heteroatoms independently selected from nitrogen, oxygen, and sulfur, wherein Cy^{B} is substituted with n instances of R^{B}. In some embodiments, Cy^{B} is a saturated 7-10 membered bridged bicyclic or fused heterocyclic ring having 1-2 heteroatoms independently selected from nitrogen, oxygen, and sulfur, wherein Cy^{B} is substituted with n instances of R^{B}. In some embodiments, Cy^{B} is a saturated 7-10 membered bridged bicyclic heterocyclic ring having 1-2 heteroatoms independently selected from nitrogen, oxygen, and sulfur, wherein Cy^{B} is substituted with n instances of R^{B}. In some embodiments, Cy^{B} is a saturated 7-8 membered bridged bicyclic heterocyclic ring having 1-2 heteroatoms independently selected from nitrogen, oxygen, and sulfur, wherein Cy^{B} is substituted with n instances of R^{B}.

In some embodiments, Cy^{B} is

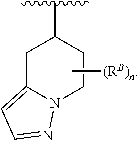

In some embodiments, Cy^{B} is

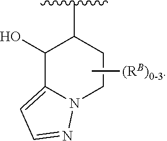

In some embodiments, Cy^{B} is

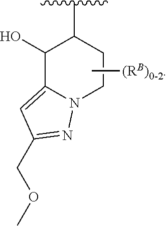

In some embodiments, Cy^{B} is

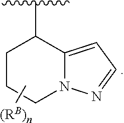

In some embodiments, Cy^{B} is

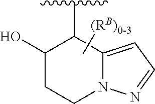

In some embodiments, Cy^{B} is

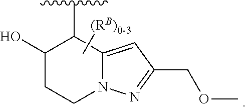

In some embodiments, Cy^{B} is

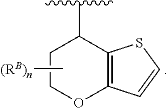

In some embodiments, Cy^{B} is

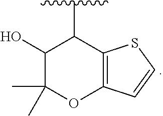 [SMILES: *C1c2sccc2OC(C)(C)C1O]

In some embodiments, Cy^{B} is

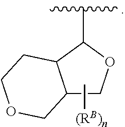

In some embodiments, Cy^{B} is

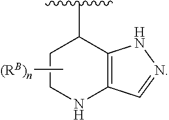

In some embodiments, Cy^{B} is

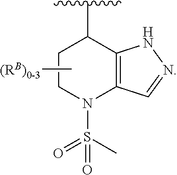

In some embodiments, Cy^{B} is a saturated or partially unsaturated 7-10 membered spirofused heterocyclic ring having 1-2 heteroatoms independently selected from nitrogen, oxygen, phosphorus, and sulfur, wherein Cy^{B} is substituted with n instances of R^{B}. In some embodiments, Cy^{B} is a saturated or partially unsaturated 7-9 membered spirofused heterocyclic ring having 1-2 heteroatoms independently selected from nitrogen, oxygen, phosphorus, and sulfur, wherein Cy^{B} is substituted with n instances of R^{B}.

In some embodiments, Cy^{B} is

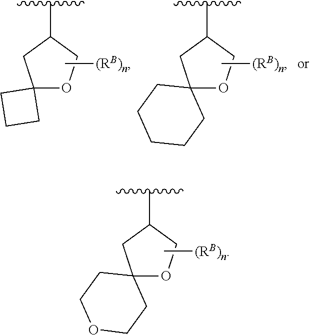

In some embodiments, Cy^{B} is

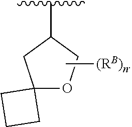

In some embodiments, Cy^{B} is

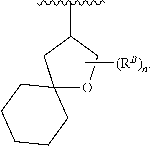

In some embodiments, Cy^{B} is

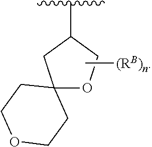

In some embodiments, Cy^{B} is

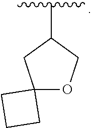 [SMILES: *C1COC2(CCC2)C1]

In some embodiments, Cy^{B} is

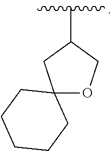 [SMILES: *C1COC2(CCCCC2)C1]

In some embodiments, Cy^{B} is

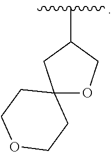 [SMILES: *C1COC2(CCOCC2)C1]

In some embodiments, Cy^{B} is selected from the groups depicted in the compounds in Table 1.

In some embodiments, Cy^{A} is phenyl, naphthyl, a 5-6 membered monocyclic heteroaryl ring having 1-4 heteroatoms independently selected from nitrogen, oxygen, phosphorus, and sulfur; an 8-12 membered bicyclic heteroaryl ring having 1-4 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; or a 10-15 membered saturated or partially unsaturated tricyclic heterocyclic ring having 1-6 heteroatoms independently selected from nitrogen, oxygen, phosphorus, and sulfur; wherein Cy^{X} is substituted with u instances of L²-X⁰.

In some embodiments, Cy^{X} is phenyl, wherein Cy^{X} is substituted with u instances of L²-X⁰. In some embodiments, Cy^{X} is naphthyl, wherein Cy^{X} is substituted with u instances of L²-X⁰. In some embodiments, Cy^{X} is a 5-6 membered monocyclic heteroaryl ring having 1-4 heteroatoms independently selected from nitrogen, oxygen, phosphorus, and sulfur, wherein Cy^{X} is substituted with u instances of L²-X⁰. In some embodiments, Cy^{X} is an 8-12 membered bicyclic heteroaryl ring having 1-4 heteroatoms independently selected from nitrogen, oxygen, phosphorus, and sulfur, wherein Cy^{X} is substituted with u instances of L²-X⁰. In some embodiments, Cy^{X} is a 7-12 membered saturated, partially unsaturated, or aromatic bicyclic heterocyclic ring having 1-4 heteroatoms independently selected from nitrogen, oxygen, phosphorus, and sulfur, wherein Cy^{X} is substituted with u instances of L²-X⁰. In some embodiments, Cy^{X} is a 10-15 membered saturated, partially unsaturated, or aromatic tricyclic heterocyclic ring having 1-6 heteroatoms independently selected from nitrogen, oxygen, phosphorus, and sulfur; wherein Cy^{X} is substituted with u instances of L²-X⁰.

In some embodiments, Cy^{X} is a 6 membered monocyclic heteroaryl ring having 1-4 heteroatoms independently selected from nitrogen, oxygen, phosphorus, and sulfur, wherein Cy^{X} is substituted with u instances of L²-X⁰. In some embodiments, Cy^{X} is a 13-15 membered saturated or partially unsaturated tricyclic heterocyclic ring having 1-6 heteroatoms independently selected from nitrogen, oxygen, phosphorus, and sulfur; wherein Cy^{X} is substituted with u instances of L²-X⁰.

In some embodiments, Cy^{X} is

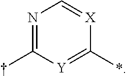

wherein [symbol] represents a bond to Q, and [symbol] represents a bond to Cy^{A}. In some embodiments, Cy^{X} is

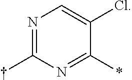

In some embodiments, Cy^{X} is

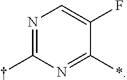

In some embodiments, Cy^{X} is

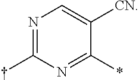

In some embodiments, Cy^{X} is

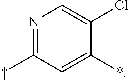

In some embodiments, Cy^{X} is

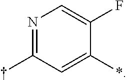

In some embodiments, Cy^{X} is

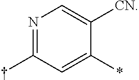

In some embodiments, Cy^{X} is

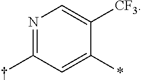

In some embodiments, Cy^{X} is

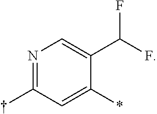

In some embodiments, Cy^{X} is

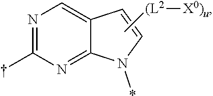

wherein [symbol] represents a bond to Q, and [symbol] represents a bond to Cy^{A}. In some embodiments Cy^{X} is

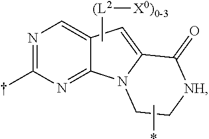

wherein [symbol] represents a bond to Q, and [symbol] represents a bond to Cy^{A}. In some embodiments Cy^{X} is

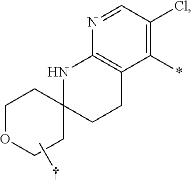

wherein [symbol] represents a bond to Q, and [symbol] represents a bond to Cy^{A}. In some embodiments Cy^{X} is

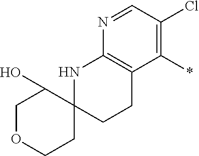

wherein [symbol] represents a bond to Cy^{A}. In some embodiments Cy^{X} is

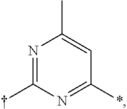

wherein [symbol] represents a bond to Q, and [symbol] represents a bond to Cy^{A}.

In some embodiments, Cy^{A} is selected from the groups depicted in the compounds in Table 1.

As defined generally above, X is N or CL²-X⁰. In some embodiments, X is N. In some embodiments, X is CL²-X⁰. In some embodiments, X (i.e., CL²-X⁰ taken together) is C—Cl. In some embodiments, X (i.e., CL²-X⁰ taken together) is C—F. In some embodiments, X (i.e., CL²-X⁰ taken together) is C—CH₃. In some embodiments, X (i.e., CL²-X⁰ taken together) is C—CHF₂. In some embodiments, X (i.e., CL²-X⁰ taken together) is C—CF₃.

In some embodiments, X is selected from the groups depicted in the compounds in Table 1.

In some embodiments, X⁰ is hydrogen, a halogen, oxo, CN, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{X}

In some embodiments, X⁰ is hydrogen, a halogen, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{N}.

In some embodiments, X⁰ is hydrogen. In some embodiments, X⁰ a halogen. In some embodiments, X⁰ is C₁₋₈ aliphatic substituted with p instances of R^{X}. In some embodiments, X⁰ is a saturated or partially unsaturated 3-14 membered monocyclic or bicyclic carbocyclic ring substituted with p instances of R^{X}. In some embodiments, X⁰ is phenyl substituted with p instances of R^{X}. In some embodiments, X⁰ is a 3-10 membered saturated or partially unsaturated monocyclic or bicyclic heterocyclic ring having 1-4 heteroatoms independently selected from nitrogen, oxygen, and sulfur, wherein said ring is substituted with p instances of R^{X}. In some embodiments, X⁰ is a 5-14 membered monocyclic or bicyclic heteroaryl ring having 1-4 heteroatoms independently selected from nitrogen, oxygen, and sulfur, wherein said ring is substituted with p instances of R^{X}.

In some embodiments, X⁰ is Cl. In some embodiments, X⁰ is F.

In some embodiments, X⁰ is selected from the groups depicted in the compounds in Table 1.

As defined generally above, Y is N or CH. In some embodiments, Y is N. In some embodiments, Y is CH.

In some embodiments, Y is selected from the groups depicted in the compounds in Table 1.

As defined generally above, Q is L¹, wherein L¹ is as defined in embodiments and classes and subclasses herein.

In some embodiments, Q is a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, —NH—, —N(R^{L})—, or —O—.

In some embodiments, Q is —NH—,

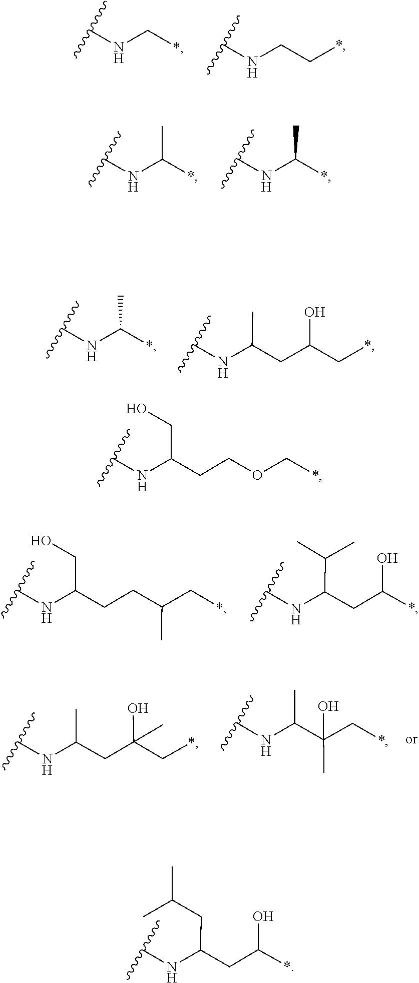

wherein

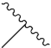

represents a covalent bond to Cy^{X} and [symbol] represents a covalent bond to Cy^{B} or Z. In some embodiments, Q is —NH—,

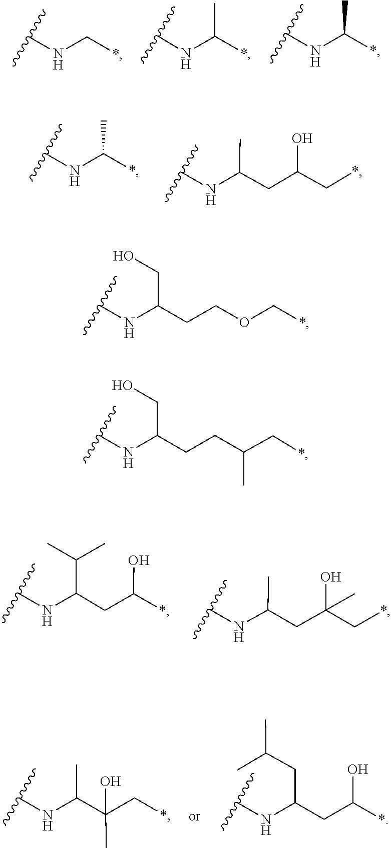

wherein

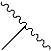

represents a covalent bond to Cy^{X} and [symbol] represents a covalent bond to Cy^{B}. In some embodiments, Q is —NH—,

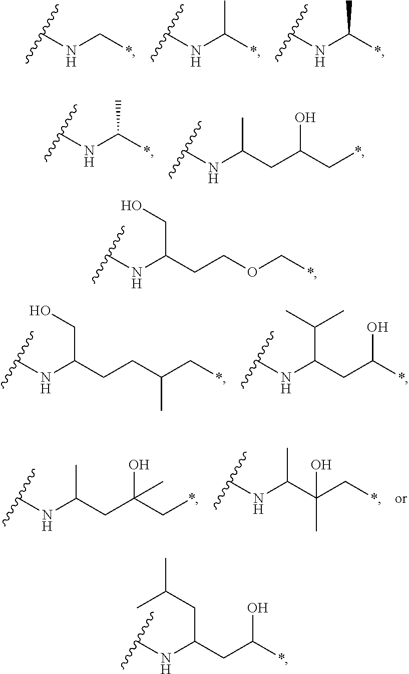

wherein

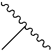

represents a covalent bond to Cy^{X} and [symbol] represents a covalent bond to Z.

In some embodiments, Q is —NH—,

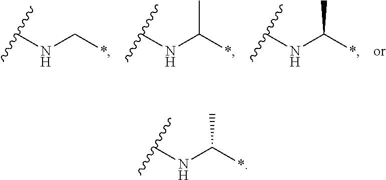

In some embodiments, Q is —NH—. In some embodiments, Q is

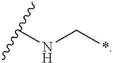

In some embodiments, Q is

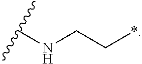

In some embodiments, Q is

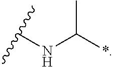

In some embodiments, Q is

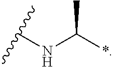

In some embodiments, Q is

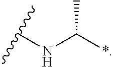

In some embodiments, Q is

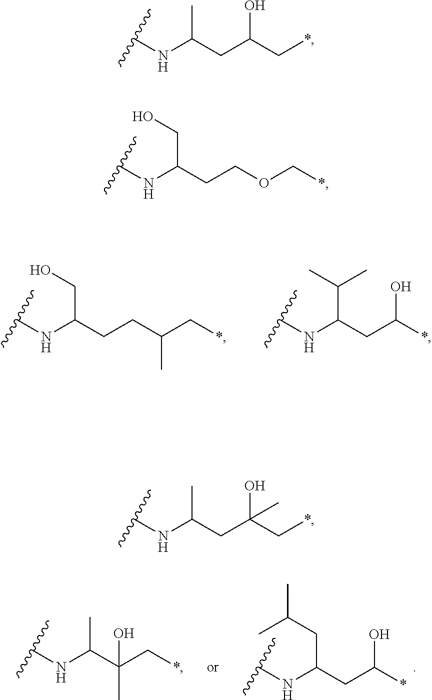

In some embodiments, Q is

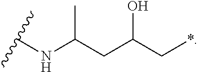

In some embodiments, Q is

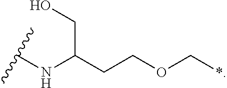

In some embodiments, Q is

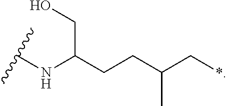

In some embodiments, Q is

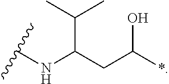

In some embodiments, Q is

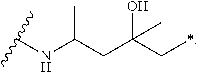

In some embodiments, Q is

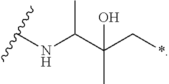

In some embodiments, Q is

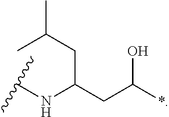

In some embodiments, Q is selected from the groups depicted in the compounds in Table 1.

In some embodiments, Z is halogen, Cy^{B} or H. In some embodiments, Z is Cy^{B} or H. In some embodiments, Z is Cy^{B} wherein Cy^{B} is as defined in embodiments and classes and subclasses herein. In some embodiments, Z is H.

In some embodiments, -Q-Z (i.e., Q and Z taken together) is

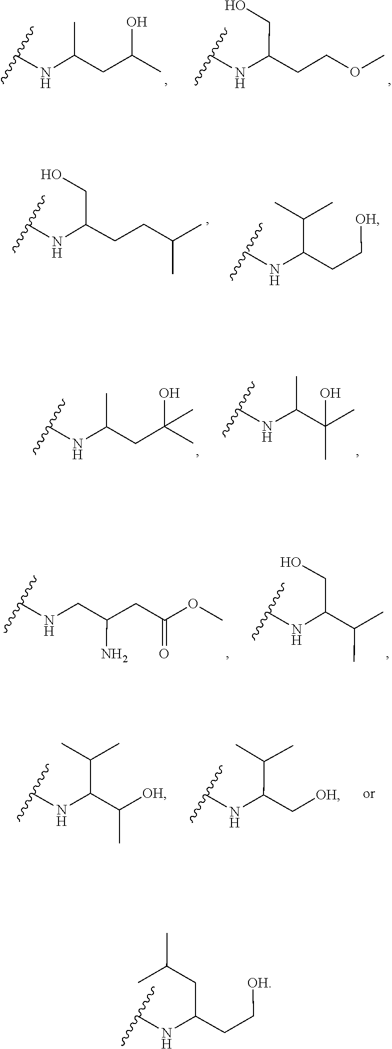

In some embodiments, -Q-Z is

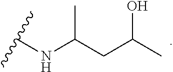 [SMILES: *NC(C)CC(C)O]

In some embodiments, -Q-Z is

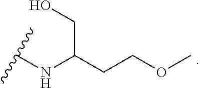 [SMILES: *NC(CO)CCOC]

In some embodiments, -Q-Z is

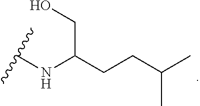 [SMILES: *NC(CO)CCC(C)C]

In some embodiments, -Q-Z is

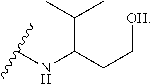 [SMILES: *NC(CCO)C(C)C]

In some embodiments, -Q-Z is

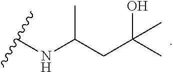 [SMILES: *NC(C)CC(C)(C)O]

In some embodiments, -Q-Z is

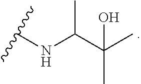 [SMILES: *NC(C)C(C)(C)O]

In some embodiments, -Q-Z is

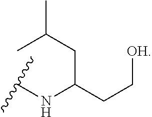 [SMILES: *NC(CCO)CC(C)C]

In some embodiments, -Q-Z is

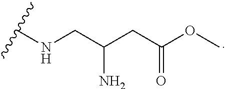 [SMILES: *NCC(N)CC(=O)OC]

In some embodiments, -Q-Z is

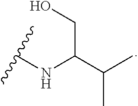 [SMILES: *NC(CO)C(C)C]

In some embodiments, -Q-Z is

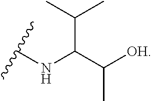 [SMILES: *NC(C(C)C)C(C)O]

In some embodiments, -Q-Z is

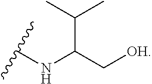 [SMILES: *NC(CO)C(C)C]

In some embodiments, Z is selected from the groups depicted in the compounds in Table 1.

As defined generally above, L¹ is a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic.

In some embodiments, L¹ is a covalent bond. In some embodiments, L¹ is a C₁₋₆ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L}) S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic.

In some embodiments, L¹ is a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, —NH—, —N(R^{L})—, or —O—.

In some embodiments, L¹ is a —NH—,

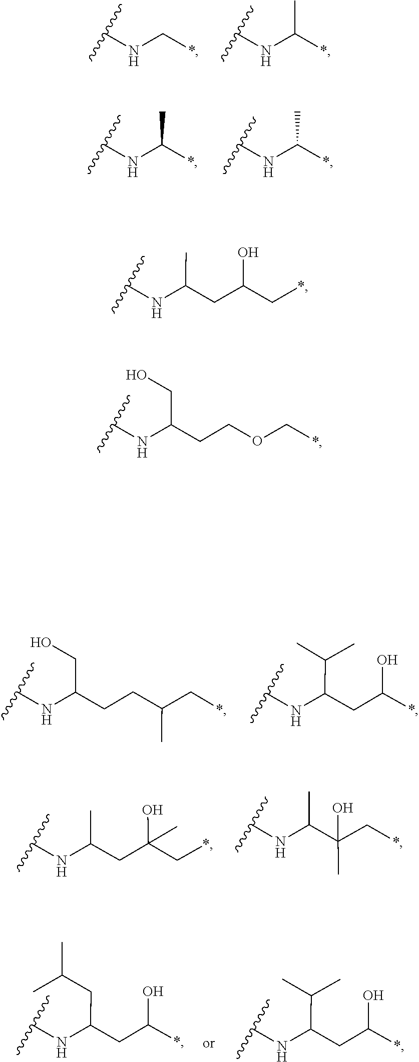

wherein

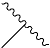

represents a covalent bond to Cy^{X} and [symbol] represents a covalent bond to Cy^{B} or Z.

In some embodiments, L is —NH—. In some embodiments, L¹ is

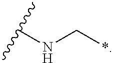

In some embodiments, L¹ is

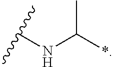

In some embodiments, L¹ is

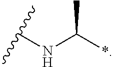

In some embodiments, L¹ is

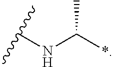

In some embodiments, L¹ is

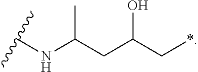

In some embodiments, L¹ is

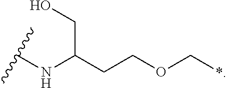

In some embodiments, L¹ is

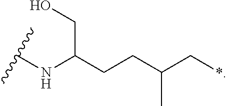

In some embodiments, L¹ is

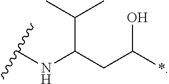

In some embodiments, L¹ is

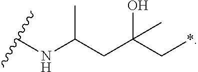

In some embodiments, L¹ is

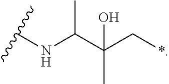

In some embodiments, L¹ is

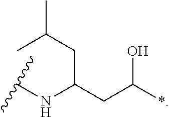

In some embodiments, L¹ is

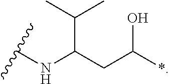

In some embodiments, L¹ is selected from the groups depicted in the compounds in Table 1.

As defined generally above, L² is a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic.

In some embodiments, L² is a covalent bond. In some embodiments, L² is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic. In some embodiments, L² is —CH₂—.

In some embodiments, each L² is selected from the groups depicted in the compounds in Table 1.

As defined generally above, each instance of R^{L} is independently R¹ or R², and is substituted by t instances of R³. In some embodiments, R^{L} is R¹. In some embodiments, R^{L} is R².

As defined generally above, each instance of R^{A}, R^{B}, and R, is independently R¹ or R² wherein R^{A} is substituted by q^{A} instances of R³, R^{B} is substituted by q^{B} instances of R³, and R^{C} is substituted by q^{X} instances of R³. In some embodiments, R^{A} is R¹. In some embodiments, R^{B} is R¹. In some embodiments, R^{X} is R¹. In some embodiments, R^{A} is R². In some embodiments, R^{B} is R². In some embodiments, R^{X} is R².

In some embodiments, each instance of R¹ (e.g., the R¹ group of R^{A}, the R¹ group of R^{B} or the R¹ group of R^{X}) is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or —P(O)R₂.

In some embodiments, each instance of R¹ (e.g., the R¹ group of R^{A}, the R¹ group of R^{B} or the R¹ group of R^{X}) is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.

In some embodiments, R¹ is oxo. In some embodiments, each R¹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.

In some embodiments, R¹ is halogen, —CN, or —NO₂. In some embodiments, R¹ is —OR, —SR, or —NR₂. In some embodiments, R¹ is —S(O)₂R, —S(O)₂NR₂, —S(O)R, —S(O)NR₂, —C(O)R, —C(O)OR, —C(O)NR₂, —C(NR)NR₂, or —C(O)N(R)OR. In some embodiments, R¹ is —S(O)₂R, —S(O)₂N(H)R, —S(O)R, —S(O)N(H)R, —C(O)R, —C(O)OR, —C(O)N(H)R, —C(NH)N(H)R, or —C(O)N(H)OR. In some embodiments, R¹ is —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R. In some embodiments, R¹ is —OC(O)R, —OC(O)N(H)R, —N(H)C(O)OR, —N(H)C(O)R, —N(H)C(NH)R, —N(H)C(O)NR₂, —N(H)C(NH)NR₂, —N(H)S(O)₂NR₂, —N(H)S(O)R, or —N(H)S(O)₂R. In some embodiments, R^{A} is halogen. In some embodiments, R^{B} is halogen. In some embodiments, R^{B} is —CN.

As defined generally above, each instance of R² (e.g., the R² group of R^{A}, the R² group of R^{B}, or the R² group of R^{X}) is independently C₁₋₇ aliphatic; —O—C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R² is C₁₋₇ aliphatic. In some embodiments, R² is —O—C₁₋₇ aliphatic. In some embodiments, R² is C₁₋₄ haloalkyl. In some embodiments, R² is phenyl. In some embodiments, R² is a 5-6 membered monocyclic heteroaryl ring having 1-4 heteroatoms independently selected from nitrogen, oxygen, and sulfur. In some embodiments, R² is an 8-10 membered bicyclic heteroaryl ring having 1-4 heteroatoms independently selected from nitrogen, oxygen, and sulfur. In some embodiments, R² is a 3-7 membered saturated or partially unsaturated monocyclic carbocyclic ring. In some embodiments, R² is a 3-7 membered saturated or partially unsaturated monocyclic heterocyclic ring having 1-2 heteroatoms independently selected from nitrogen, oxygen, and sulfur. In some embodiments, R² is a 7-12 membered saturated or partially unsaturated bicyclic heterocyclic ring having 1-4 heteroatoms independently selected from nitrogen, oxygen, and sulfur.

In some embodiments, R^{A} is C₁₋₇ aliphatic. In some embodiments, R^{A} is halogen. In some embodiments, R^{A} is F. In some embodiments, R^{A} is Cl. In some embodiments, R^{A} is —C(CH₃)₂OH. In some such embodiments, R^{A} is —CH₃. In some embodiments, R^{A} is —OR. In some embodiments, R^{A} is oxo. In some embodiments, R^{A} is —C(CH₃)₃. In some such embodiments, R^{A} is —CF₃. In some such embodiments, R^{A} is —CHF₂.

In some embodiments, R^{A} is a 3-7 membered saturated or partially unsaturated monocyclic heterocyclic ring having 1-2 heteroatoms independently selected from nitrogen, oxygen, and sulfur. In some embodiments, R^{A} is a 4-6 membered saturated or partially unsaturated monocyclic heterocyclic ring having 1-2 heteroatoms independently selected from nitrogen, oxygen, and sulfur. In some embodiments, R^{A} is a 5-6 membered saturated or partially unsaturated monocyclic heterocyclic ring having 1-2 heteroatoms independently selected from nitrogen, oxygen, and sulfur. In some embodiments, R^{A} is a 5-membered saturated or partially unsaturated monocyclic heterocyclic ring having 1-2 heteroatoms independently selected from nitrogen, oxygen, and sulfur. In some embodiments, R^{A} is a 6-membered saturated or partially unsaturated monocyclic heterocyclic ring having 1-2 heteroatoms independently selected from nitrogen, oxygen, and sulfur.

In some embodiments, R^{A} is

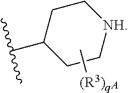

In some embodiments, R^{A} is

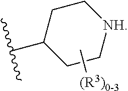

In some embodiments, R^{A} is

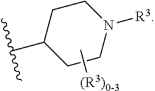

In some embodiments, R^{A} is

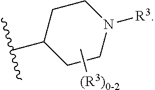

In some embodiments, R^{A} is

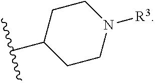

In some embodiments, R^{A} is

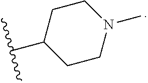 [SMILES: *C1CCN(C)CC1]

In some embodiments, R^{A} is

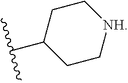 [SMILES: *C1CCNCC1]

In some embodiments, R^{A} is —NR₂. In some embodiments, R^{A} is —N(CH₃)₂.

In some embodiments, each R^{A} is selected from the groups depicted in the compounds in Table 1.

In some embodiments, R^{B} is C₁₋₇ aliphatic. In some embodiments, R^{B} is halogen. In some embodiments, R^{B} is F. In some embodiments, R^{B} is Cl. In some such embodiments, R^{B} is —CH₃. In some embodiments, R^{B} is selected from —CH₃, —CH₂CH₃, —CH(CH₃)₂,

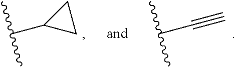

In some embodiments, R^{B} is C₁₋₇ aliphatic substituted with R³. In some embodiments, R^{B} is C₁₋₇ aliphatic substituted with R³, wherein R³ is —OR. In some embodiments, R^{B} is C₁₂ aliphatic substituted with R³, wherein R³ is —OR. In some embodiments, R^{B} is —CH₂OH. In some embodiments, R^{B} is oxo. In some embodiments, R^{B} is —OR, wherein R is C₁₋₆ aliphatic. In some embodiments, R^{B} is —OCH₃. In some embodiments, R^{B} is oxo. In some embodiments, R^{B} is —CN.

In some embodiments, R^{B} is —OH.

In some embodiments, each R^{B} is selected from the groups depicted in the compounds in Table 1.

In some embodiments, R^{X} is C₁₋₇ aliphatic. In some embodiments, R^{X} is halogen. In some embodiments, R^{X} is F. In some embodiments, R^{X} is Cl. In some such embodiments, R^{X} is —CH₃ or —C(CH₃)₃. In some embodiments, R^{X} is —CH₂C(CH₃)₃. In some embodiments, R^{X} is C₁₋₇ aliphatic substituted with R³. In some embodiments, R^{X} is C₁₋₇ aliphatic substituted with R³, wherein R³ is —OR. In some embodiments, R^{X} is C₁₋₂ aliphatic substituted with R³, wherein R³ is —OR. In some embodiments, R^{X} is —CH₂OCH₃. In some embodiments, R^{X} is

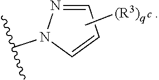

In some embodiments, R^{X} is —N(H)C(O)CH₃. In some embodiments, R^{X} is —C(O)OR. In some embodiments, R^{X} is —C(O)OR, wherein R is C₁₋₆ aliphatic. In some embodiments, R^{X} is —C(O)OCH₂CH₃. In some embodiments, R^{X} is C₁₋₇ aliphatic substituted with R³, wherein R³ is halogen. In some embodiments, R^{X} is C₁₋₇ aliphatic substituted with R³, wherein R³ is fluorine. In some embodiments, R^{X} is —CF₃. In some embodiments, R^{X} is oxo. In some embodiments, R^{X} is —OR substituted with R³. In some embodiments, R^{X} is —OR substituted with R³, wherein R is C₁₋₆ aliphatic and R³ is —OR. In some embodiments, R^{X} is —OCH₂CH₂OH.

In some embodiments, each R^{X} is selected from the groups depicted in the compounds in Table 1.

As defined generally above, each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R³ is oxo. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —S(O)NR₂. In some embodiments, R³ is —S(O)₂F. In some embodiments, R³ is —OS(O)₂F. In some embodiments, R³ is —C(O)R. In some embodiments, R³ is —C(O)OR. In some embodiments, R³ is —C(O)NR₂. In some embodiments, R³ is —C(NR)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(NR)R. In some embodiments, R³ is —N(R)C(O)NR₂. In some embodiments, R³ is —N(R)C(NR)NR₂. In some embodiments, R³ is —N(R)S(O)₂NR₂. In some embodiments, R³ is —N(R)S(O)R. In some embodiments, R³ is —N(R)S(O)₂R. In some embodiments, R³ is an optionally substituted C₁₋₆ aliphatic group. In some embodiments, R³ is an optionally substituted phenyl. In some embodiments, R³ is an optionally substituted 3-7 membered saturated or partially unsaturated heterocyclic ring having 1-2 heteroatoms independently selected from nitrogen, oxygen, and sulfur. In some embodiments, R³ is an optionally substituted 5-6 membered heteroaryl ring having 1-4 heteroatoms independently selected from nitrogen, oxygen, and sulfur.

In some embodiments, each R³ is independently halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, each R³ is independently halogen, —CN, or —NO₂.

In some embodiments, each R³ is independently —OR, —SR, or —NR₂.

In some embodiments, each R³ is independently —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, or —C(O)N(R)OR.

In some embodiments, each R³ is independently —S(O)₂R, —S(O)₂N(H)R, —S(O)R, —S(O)N(H)R, —S(O)₂F, —OS(O)₂F, —C(O)R, —C(O)OR, —C(O)N(H)R, —C(NH)NR₂, or —C(O)N(H)OR.

In some embodiments, each R³ is independently OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.

In some embodiments, each R³ is independently —OC(O)R, —OC(O)N(H)R, —N(H)C(O)OR, —N(H)C(O)R, —N(H)C(NH)R, —N(H)C(O)NR₂, —N(H)C(NH)NR₂, —N(H)S(O)₂NR₂, —N(H)S(O)R, or —N(H)S(O)₂R.

In some embodiments, each R³ is independently an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R³—CH₃.

In some embodiments, each R³ is selected from the groups depicted in the compounds in Table 1.

As defined generally above, each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur. In some embodiments, R is optionally substituted C₁₋₆ aliphatic. In some embodiments, R is an optionally substituted saturated or partially unsaturated 3-7 membered carbocyclic ring. In some embodiments, R is optionally substituted phenyl. In some embodiments, R is an optionally substituted 3-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur.

In some embodiments, each instance of R is independently hydrogen or C₁₋₆ alkyl. In some embodiments, each instance of R is independently hydrogen or methyl.

As defined generally above, m is 0, 1, 2, 3, or 4. In some embodiments, m is 0. In some embodiments, m is 1. In some embodiments, m is 2. In some embodiments, m is 3. In some embodiments, m is 4. In some embodiments, m is 0 or 1. In some embodiments, m is 0, 1, or 2. In some embodiments, m is 0, 1, 2, or 3. In some embodiments, m is 1 or 2. In some embodiments, m is 1, 2, or 3. In some embodiments, m is 1, 2, 3, or 4. In some embodiments, m is 2 or 3. In some embodiments, m is 2, 3, or 4. In some embodiments, m is 3 or 4. In some embodiments, m is selected from the values represented in the compounds in Table 1.

As defined generally above, n is 0, 1, 2, 3, or 4. In some embodiments, n is 0. In some embodiments, n is 1. In some embodiments, n is 2. In some embodiments, n is 3. In some embodiments, n is 4. In some embodiments, n is 0 or 1. In some embodiments, n is 0, 1, or 2. In some embodiments, n is 0, 1, 2, or 3. In some embodiments, n is 1 or 2. In some embodiments, n is 1, 2, or 3. In some embodiments, n is 1, 2, 3, or 4. In some embodiments, n is 2 or 3. In some embodiments, n is 2, 3, or 4. In some embodiments, n is 3 or 4. In some embodiments, n is selected from the values represented in the compounds in Table 1.

As defined generally above, p is 0, 1, 2, 3, or 4. In some embodiments, p is 0. In some embodiments, p is 1. In some embodiments, p is 2. In some embodiments, p is 3. In some embodiments, p is 4. In some embodiments, p is 0 or 1. In some embodiments, p is 0, 1, or 2. In some embodiments, p is 0, 1, 2, or 3. In some embodiments, p is 1 or 2. In some embodiments, p is 1, 2, or 3. In some embodiments, p is 1, 2, 3, or 4. In some embodiments, p is 2 or 3. In some embodiments, p is 2, 3, or 4. In some embodiments, p is 3 or 4. In some embodiments, p is selected from the values represented in the compounds in Table 1.

As defined generally above, q^{A} is 0, 1, 2, 3, or 4. In some embodiments, q^{A} is 0. In some embodiments, q^{A} is 1. In some embodiments, q^{A} is 2. In some embodiments, q^{A} is 3. In some embodiments, q^{A} is 4. In some embodiments, q^{A} is 0 or 1. In some embodiments, q^{A} is 0, 1, or 2. In some embodiments, q^{A} is 0, 1, 2, or 3. In some embodiments, q^{A} is 1 or 2. In some embodiments, q^{A} is 1, 2, or 3. In some embodiments, q^{A} is 1, 2, 3, or 4. In some embodiments, q^{A} is 2 or 3. In some embodiments, q^{A} is 2, 3, or 4. In some embodiments, q^{A} is 3 or 4. In some embodiments, q^{A} is selected from the values represented in the compounds in Table 1.

As defined generally above, q^{B} is 0, 1, 2, 3, or 4. In some embodiments, q^{B} is 0. In some embodiments, q^{B} is 1. In some embodiments, q^{B} is 2. In some embodiments, q^{B} is 3. In some embodiments, q^{B} is 4. In some embodiments, q^{B} is 0 or 1. In some embodiments, q^{B} is 0, 1, or 2. In some embodiments, q^{B} is 0, 1, 2, or 3. In some embodiments, q^{B} is 1 or 2. In some embodiments, q^{B} is 1, 2, or 3. In some embodiments, q^{B} is 1, 2, 3, or 4. In some embodiments, q^{B} is 2 or 3. In some embodiments, q^{B} is 2, 3, or 4. In some embodiments, q^{B} is 3 or 4. In some embodiments, q^{B} is selected from the values represented in the compounds in Table 1.

As defined generally above, q^{X} is 0, 1, 2, 3, or 4. In some embodiments, q^{X} is 0. In some embodiments, q^{X} is 1. In some embodiments, q^{X} is 2. In some embodiments, q^{X} is 3. In some embodiments, q^{X} is 4. In some embodiments, q^{X} is 0 or 1. In some embodiments, q^{X} is 0, 1, or 2. In some embodiments, q^{X} is 0, 1, 2, or 3. In some embodiments, q^{X} is 1 or 2. In some embodiments, q^{X} is 1, 2, or 3. In some embodiments, q^{X} is 1, 2, 3, or 4. In some embodiments, q^{X} is 2 or 3. In some embodiments, q^{X} is 2, 3, or 4. In some embodiments, q^{X} is 3 or 4. In some embodiments, q^{X} is selected from the values represented in the compounds in Table 1.

As defined generally above, r is 0, 1, 2, 3, or 4. In some embodiments, r is 0. In some embodiments, r is 1. In some embodiments, r is 2. In some embodiments, r is 3. In some embodiments, r is 4. In some embodiments, r is 0 or 1. In some embodiments, r is 0, 1, or 2. In some embodiments, r is 0, 1, 2, or 3. In some embodiments, r is 1 or 2. In some embodiments, r is 1, 2, or 3. In some embodiments, r is 1, 2, 3, or 4. In some embodiments, r is 2 or 3. In some embodiments, r is 2, 3, or 4. In some embodiments, r is 3 or 4. In some embodiments, r is selected from the values represented in the compounds in Table 1.

As defined generally above, t is 0, 1, 2, 3, or 4. In some embodiments, t is 0. In some embodiments, t is 1. In some embodiments, t is 2. In some embodiments, t is 3. In some embodiments, t is 4. In some embodiments, t is 0 or 1. In some embodiments, t is 0, 1, or 2. In some embodiments, t is 0, 1, 2, or 3. In some embodiments, t is 1 or 2. In some embodiments, t is 1, 2, or 3. In some embodiments, t is 1, 2, 3, or 4. In some embodiments, t is 2 or 3. In some embodiments, t is 2, 3, or 4. In some embodiments, t is 3 or 4. In some embodiments, t is selected from the values represented in the compounds in Table 1.

As defined generally above, u is 0, 1, 2, 3, or 4. In some embodiments, u is 0. In some embodiments, u is 1. In some embodiments, u is 2. In some embodiments, u is 3. In some embodiments, u is 4. In some embodiments, u is 0 or 1. In some embodiments, u is 0, 1, or 2. In some embodiments, u is 0, 1, 2, or 3. In some embodiments, u is 1 or 2. In some embodiments, u is 1, 2, or 3. In some embodiments, u is 1, 2, 3, or 4. In some embodiments, u is 2 or 3. In some embodiments, u is 2, 3, or 4. In some embodiments, u is 3 or 4. In some embodiments, u is selected from the values represented in the compounds in Table 1.

Examples of compounds described herein include those listed in the Tables and exemplification herein, or a pharmaceutically acceptable salt, stereoisomer, or mixture of stereoisomers thereof. In some embodiments, the present disclosure comprises a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

Lengthy table referenced here
US20260193248A1-20260709-T00001
Please refer to the end of the specification for access instructions.

In chemical structures in Table 1, above, when a stereocenter is depicted with a dashed or wedged bond and labeled “abs” (or unlabeled), the compound is essentially a single isomer at that stereocenter (rather than an equimolar mixture), and the absolute stereochemistry is as shown in the chemical structure. (See, for example, the structure of compound I-1.) When a stereocenter is depicted with a dashed or wedged bond and also labeled “or1” or “or2,” the compound is a single isomer at that stereocenter, but the absolute stereochemistry at that stereocenter has not been determined. (See, for example, the structure of compound I-36.) Similarly, where a compound name contains “rel-” it is a single isomer at the denoted stereocenter(s), but the absolute stereochemistry at those stereocenters has not been determined. When a stereocenter is depicted with a dashed or wedged bond and also labeled “and1” or “&1”, the compound is a mixture of two isomers at that stereocenter: the structure as drawn, and the isomer in which that stereogenic center has the opposite configuration. (See, for example, the structure of an intermediate in Example 15.)

Certain compounds depicted in Table 1, above, exist in solution at room temperature as non-interconverting atropisomers across a biaryl bond. When one of the atoms of a biaryl bond is labeled as “or1”, this signifies that the compound exists in solution at room temperature as non-interconverting atropisomers, and the compound is essentially a single atropisomer (rather than an equimolar mixture).

In some embodiments, the present disclosure provides a compound in Table 1, above, wherein the compound is denoted as having a Biochemical CDK4 Caliper IC₅₀ of “A”. In some embodiments, the present disclosure provides a compound in Table 1, above, wherein the compound is denoted as having a Biochemical CDK4 Caliper IC₅₀ of “A” or “B”. In some embodiments, the present disclosure provides a compound in Table 1, above, wherein the compound is denoted as having a Biochemical CDK4 Caliper IC₅₀ of “A” or “B” or “C”.

In some embodiments, the present disclosure provides a compound in Table 1, above, wherein the compound is denoted as having a Cell nanoBRET IC₅₀ of “A”. In some embodiments, the present disclosure provides a compound in Table 1, above, wherein the compound is denoted as having a Cell nanoBRET IC₅₀ of “A” or “B”. In some embodiments, the present disclosure provides a compound in Table 1, above, wherein the compound is denoted as having a Cell nanoBRET IC₅₀ of “A” or “B” or “C”.

### 4. General Methods of Providing the Present Compounds

The compounds described herein may be prepared or isolated in general by synthetic and/or semi-synthetic methods known to those skilled in the art for analogous compounds and by methods described in detail in the Examples, herein.

### 5. Uses, Formulation, and Administration

### Pharmaceutically Acceptable Compositions

According to another embodiment, the present disclosure provides a composition comprising a compound described herein, or a pharmaceutically acceptable derivative thereof, and a pharmaceutically acceptable carrier, adjuvant, or vehicle. In some embodiments, the present disclosure provides a pharmaceutical composition comprising a compound described herein, and a pharmaceutically acceptable carrier. The amount of compound in compositions described herein is such that is effective to measurably inhibit a CDK4 protein kinase, or a mutant thereof, in a biological sample or in a patient. In certain embodiments, the amount of compound in compositions described herein is such that it is effective to measurably inhibit a CDK4 protein kinase, or a mutant thereof, in a biological sample or in a patient. In certain embodiments, a composition described herein is formulated for administration to a patient in need of such composition. In some embodiments, a composition described herein is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound described herein that, upon administration to a recipient, is capable of providing, either directly or indirectly, a compound described herein or an inhibitorily active metabolite or residue thereof.

As used herein, the term “inhibitorily active metabolite or residue thereof” means that a metabolite or residue thereof is also an inhibitor of a CDK4 protein kinase, or a mutant thereof.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions described herein may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions described herein are formulated for oral administration. Such formulations may be administered with or without food. In some embodiments, pharmaceutically acceptable compositions described herein are administered without food. In other embodiments, pharmaceutically acceptable compositions described herein are administered with food.

The amount of compounds described herein that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

The precise dose to be employed in the compositions will also depend on the route of administration and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R″′R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R″′, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the inhibition of a kinase or a mutant thereof. In some embodiments, the kinase inhibited by the compounds and compositions described herein is a cyclin dependent kinase (CDK). In some embodiments, the kinase inhibited by the compounds and compositions described herein is one or more of CDK1, CDK2, CDK4, and CDK6. In some embodiments, the kinase inhibited by the compounds and compositions described herein is CDK4.

Compounds or compositions of the disclosure can be useful in applications that benefit from inhibition of CDK4 enzymes. For example, CDK4 inhibitors described herein are useful for the treatment of proliferative diseases generally.

Cyclin D and its associated kinase CDK4 are known to be factors in tumorigenesis and proliferation in many cancer types, including melanoma, upper gastrointestinal tract cancer, head and neck cancer, breast cancer, lung cancer, and bladder cancer (Cerami E et al. (2012) Cancer Discov 2 (5), 401-4; Gao J et al. (2013) Science Signaling 6 (269), 11). In breast cancer, cyclin D and CDK4 have been correlated with worse clinical outcomes (Lundgren K et al. (2012) Breast Cancer Res 14 (2), R57).

Studies in transgenic animals had shown that cyclin D1 deficiency strongly and specifically impairs mammary epithelial proliferation, suggesting a key role for the CDK4/6 pathway in this tissue (Sicinski P et al. (1995) Cell 82 (4), 621-30). Moreover, cyclin D1 and CDK4 are essential for the formation and growth of several murine mammary tumors (Yu Q et al. (2001) Nature 411 (6841), 1017-21; Yu Q et al. (2006) Cancer Cell 9 (1), 23-32; Jeselsohn R et al. (2010) Cancer Cell 17 (1), 65-76). In estrogen receptor-positive human breast cancer cell lines, combined inhibition of CDK4/6 and ER (with endocrine therapy) reduces tumor cell proliferation synergistically (Finn R S et al. (2009) Breast Cancer Res 11 (5), R77)

In addition, numerous cancers demonstrate amplifications of CDK4 (most notably liposarcoma and glioblastoma) (Cerami E et al. (2012) Cancer Discov 2 (5), 401-4.) Certain genomic translocations and specific gene mutations can also markedly increase cyclin D levels in tumor cells. The resultant increase in CDK4 activity might be expected to confer sensitivity to CDK4 inhibitors, and this has indeed been shown to be the case (Gong X et al. (2017) Cancer Cell 32 (6), 761-776 e6.)

The activity of a compound described herein as an inhibitor of an CDK kinase, for example, CDK4, or a mutant thereof, may be assayed in vitro, in vivo or in a cell line. In vitro assays include assays that determine inhibition of either the phosphorylation activity and/or the subsequent functional consequences, or ATPase activity of activated CDK4, or a mutant thereof. Alternative in vitro assays quantitate the ability of the inhibitor to bind to CDK4. Inhibitor binding may be measured by radiolabeling the inhibitor prior to binding, isolating the inhibitor/CDK4 complex and determining the amount of radiolabel bound. Alternatively, inhibitor binding may be determined by running a competition experiment where new inhibitors are incubated with CDK4 bound to known radioligands. Representative in vitro and in vivo assays useful in assaying an CDK4 inhibitor include those described and disclosed in the patent and scientific publications described herein. Detailed conditions for assaying a compound described herein as an inhibitor of CDK4, or a mutant thereof, are set forth in the Examples below.

### Treatment of Disorders

Provided compounds are inhibitors of CDK4 and are therefore useful for treating one or more disorders associated with activity of CDK4 or mutants thereof. Thus, in certain embodiments, the present disclosure provides a method of treating an CDK4-mediated disorder in a subject comprising administering a therapeutically effective amount of a compound described herein, or a pharmaceutically acceptable salt thereof, or a pharmaceutically acceptable composition of either of the foregoing, to a subject in need thereof. In certain embodiments, the present disclosure provides a method of treating an CDK4-mediated disorder in a subject comprising administering a therapeutically effective amount of a compound described herein, or a pharmaceutically acceptable composition thereof, to a subject in need thereof.

As used herein, the term “CDK4-mediated” disorders, diseases, and/or conditions means any disease or other deleterious condition in which CDK4 or a mutant thereof is known to play a role. Accordingly, another embodiment of the present disclosure relates to treating or lessening the severity of one or more diseases in which CDK4, or a mutant thereof, is known to play a role. Such CDK4-mediated disorders include but are not limited to proliferative disorders (e.g. cancer).

In some embodiments, the present disclosure provides a method for treating one or more disorders, wherein the disorders are selected from proliferative disorders and craniosynostotic syndromes, said method comprising administering to a patient in need thereof, a therapeutically effective amount of a compound of the present disclosure, or a pharmaceutically acceptable salt thereof, or a pharmaceutically acceptable composition of either of the foregoing. In some embodiments, the present disclosure provides a method for treating one or more disorders, wherein the disorders are selected from proliferative disorders and craniosynostotic syndromes, said method comprising administering to a patient in need thereof, a therapeutically effective amount of a compound of the present disclosure, or a pharmaceutically acceptable composition thereof.

In some embodiments, the disorder is associated with CDK4 signaling. CDK4 is known to have multiple upstream and downstream signaling pathways and inhibition of CDK4 can be used to treat disorders associated with aberrant signaling within those pathways. In some embodiments, the disorder is associated with cyclin D, cyclin D1, or retinoblastoma protein (R^{B}) signaling.

In some embodiments, the method of treatment comprises the steps of: i) identifying a subject in need of such treatment; (ii) providing a disclosed compound, or a pharmaceutically acceptable salt thereof, and (iii) administering said provided compound in a therapeutically effective amount to treat, suppress and/or prevent the disease state or condition in a subject in need of such treatment.

In some embodiments, the method of treatment comprises the steps of: i) identifying a subject in need of such treatment; (ii) providing a composition comprising a disclosed compound, or a pharmaceutically acceptable salt thereof, and (iii) administering said composition in a therapeutically effective amount to treat, suppress and/or prevent the disease state or condition in a subject in need of such treatment.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Proliferative Disorders

In some embodiments, the disorder is a proliferative disorder. In some embodiments, the proliferative disorder is cancer. In some embodiments, the proliferative disorder is ovarian cancer, breast cancer, lung cancer, colorectal cancer, or a combination thereof. In some embodiments, the proliferative disorder is a leukemia. In some embodiments, the proliferative disorder is breast cancer. In some embodiments, the proliferative disorder is a lung cancer. In some embodiments, the proliferative disorder is colorectal cancer.

In some embodiments, the proliferative disorder is breast cancer, prostate cancer, lung squamous cell carcinoma, thyroid cancer, gastric cancer, ovarian cancer, rectal cancer, endometrial carcinoma, non-small cell lung cancer, or bladder cancer. In some embodiments, the proliferative disorder is intrahepatic cholangiocarcinoma, hepatocellular carcinoma, breast cancer, prostate cancer, lung squamous cell carcinoma, thyroid cancer, gastric cancer, or ovarian cancer. In some embodiments, the proliferative disorder is gastric cancer, breast cancer, triple negative breast cancer, or rectal cancer. In some embodiments, the proliferative disorder is endometrial carcinoma, non-small cell lung cancer, lung squamous cell carcinoma, gastric cancer, breast cancer, or urothelial cancer.

In some embodiments, the disorder is ovarian cancer, endometrial cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, or pancreatic cancer. In some embodiments, the bladder cancer is urothelial carcinoma. In some embodiments, the liver cancer is hepatocellular carcinoma. In some embodiments, the lung cancer is lung squamous cell carcinoma or non-small cell lung cancer. In some embodiments, the laryngeal cancer is laryngeal squamous cell carcinoma. In some embodiments, the skin cancer is melanoma.

In some embodiments, the proliferative disorder is associated with a deregulation of CDK4 or cyclin D. In some embodiments, the deregulation of CDK4 is an overexpression of CDK4 or cyclin D. In some embodiments, the deregulation of cyclin D is an overexpression of CDK4 or cyclin D. In some embodiments, the proliferative disorder is associated with a deregulation of CDK4 and cyclin D. In some embodiments, the deregulation of CDK4 and cyclin D is an overexpression of CDK4 and cyclin D.

In some embodiments, the proliferative disorder is associated with one or more activating mutations in CDK4. In some embodiments, the activating mutation in CDK4 is a mutation to one or more of the intracellular kinase domain and the extracellular domain. In some embodiments, the activating mutation in CDK4 is a mutation to the intracellular kinase domain.

### Routes of Administration and Dosage Forms

The compounds and compositions, according to the methods described herein, may be administered using any amount and any route of administration effective for treating or lessening the severity of the disorder (e.g. a proliferative disorder or craniosynostotic syndrome). The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions described herein can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds described herein may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the present disclosure relates to a method of inhibiting protein kinase activity in a biological sample comprising the step of contacting said biological sample with a compound described herein, or a composition comprising said compound.

According to another embodiment, the present disclosure relates to a method of inhibiting activity of CDK4, or a mutant thereof, in a biological sample comprising the step of contacting said biological sample with a compound described herein, or a composition comprising said compound. In certain embodiments, the present disclosure relates to a method of reversibly inhibiting CDK4, or a mutant thereof, activity in a biological sample comprising the step of contacting said biological sample with a compound described herein, or a composition comprising said compound.

In another embodiment, the present disclosure provides a method of selectively inhibiting CDK4 over one or more of CDK1, CDK2, CDK5, CDK6, and CDK9. In some embodiments, a compound described herein is more than 5-fold selective over CDK1, CDK2, CDK5, CDK6, and CDK9. In some embodiments, a compound described herein is more than 10-fold selective over CDK1, CDK4, CDK5, CDK6, and CDK9. In some embodiments, a compound described herein is more than 50-fold selective over CDK1, CDK2, CDK5, CDK6, sand CDK9. In some embodiments, a compound described herein is more than 100-fold selective over CDK1, CDK2, CDK5, CDK6, and CDK9. In some embodiments, a compound described herein is more than 200-fold selective over CDK1, CDK2, CDK5, CDK6, and CDK9.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of CDK4 (or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound described herein, or a composition comprising said compound.

According to another embodiment, the present disclosure relates to a method of inhibiting activity of CDK4, or a mutant thereof, in a patient comprising the step of administering to said patient a compound described herein, or a composition comprising said compound. According to certain embodiments, the present disclosure relates to a method of reversibly inhibiting activity of one or more of CDK4, or a mutant thereof, in a patient comprising the step of administering to said patient a compound described herein, or a composition comprising said compound.

According to another embodiment, the present disclosure provides a method for treating a disorder mediated by CDK4, or a mutant thereof, in a patient in need thereof, comprising the step of administering to said patient a compound described herein or a pharmaceutically acceptable composition thereof. Such disorders are described in detail herein. In some embodiments, the present disclosure provides a method for treating a disorder mediated by CDK4, or a mutant thereof, in a patient in need thereof, comprising the step of administering to said patient a compound described herein or a pharmaceutically acceptable composition thereof, wherein the compound reversibly inhibits the CDK4, or a mutant thereof.

According to another embodiment, the present disclosure provides a method of inhibiting signaling activity of CDK4, or a mutant thereof, in a subject, comprising administering a therapeutically effective amount of a compound described herein, or a pharmaceutically acceptable composition thereof, to a subject in need thereof. In some embodiments, the present disclosure provides a method of inhibiting CDK4 signaling activity in a subject, comprising administering a therapeutically effective amount of a compound described herein, or a pharmaceutically acceptable composition thereof, to a subject in need thereof.

In some embodiments, the present disclosure provides a method for treating a disorder mediated by CDK4, or a mutant thereof, in a patient in need thereof, comprising the step of administering to said patient a compound described herein or a pharmaceutically acceptable composition thereof, wherein the compound reversibly inhibits the CDK4, or a mutant thereof.

The compounds described herein can also inhibit CDK4 function through incorporation into agents that catalyze the destruction of CDK4. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of CDK4 to the E3 ligase will thus result in the destruction of the CDK4 protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions described herein. As used herein, additional therapeutic agents that are normally administered to treat a particular disease, or condition, are known as “appropriate for the disease, or condition, being treated.”

Accordingly, in certain embodiments, the method of treatment comprises administering the compound or composition described herein in combination with one or more additional therapeutic agents. In certain other embodiments, the methods of treatment comprise administering the compound or composition described herein as the only therapeutic agent.

In some embodiments, the one or more additional therapeutic agents is selected from antibodies, antibody-drug conjugates, kinase inhibitors, immunomodulators, and histone deacetylase inhibitors. In some embodiments, the one or more additional therapeutic agent is selected from the following agents, or a pharmaceutically acceptable salt thereof: BCR-ABL inhibitors: e.g. imatinib, inilotinib, nilotinib, dasatinib, bosutinib, ponatinib, bafetinib, danusertib, saracatinib, PF03814735; ALK inhibitors (see Dardaei et al, 2018, NatMed.; 24(4):512-517): e.g. crizotinib, NVP-TAE684, ceritinib, alectinib, brigatinib, entrecinib, lorlatinib; BRAF inhibitors (see Prahallad et al, 2015, Cell Rep. 12, 1978-1985): e.g. vemurafenib, dabrafenib; FGFR inhibitors: e.g. infigratinib, dovitinib, erdafitinib, BLU-554, AZD4547; FLT3 inhibitors: e.g. sunitinib, midostaurin, tanutinib, sorafenib, lestaurtinib, quizartinib, and crenolanib, MEK Inhibitors (see Fedele et al, 2018, BioRxiv 307876; Torres-Ayuso et al, 2018, Cancer Discov. 8, 1210-1212; and Wong et al, 2016, Oncotarget. 2016 Oct. 4; 7(40): 65676-65695): e.g. trametinib, cobimetinib, binimetinib, selumetinib; ERK inhibitors: e.g. ulixertinib, MK-8353, LY-3214996; VEGF receptor inhibitors: e.g. bevacizumab, axitinib, aflibercept, brivanib, motesanib, pasireotide, sorafenib; Tyrosine kinase inhibitors: e.g. erlotinib, linifanib, sunitinib, pazopanib; Epidermal growth factor receptor (EGFR) inhibitors: gefitnib, osimertinib, cetuximab, panitumumab; HER2 receptor inhibitors: e.g. trastuzumab, neratinib, lapatinib, lapatinib; MET inhibitors: e.g. crizotinib, cabozantinib; CD20 antibodies: e.g. rituximab, tositumomab, ofatumumab; DNA Synthesis inhibitors: e.g. capecitabine, gemcitabine, nelarabine, hydroxycarbamide; Antineoplastic agents: e.g. oxaliplatin, cisplatin; HER dimerization inhibitors: e.g. pertuzumab; Human Granulocyte colony-stimulating factor (G-CSF) modulators: e.g. filgrastim, Immunomodulators: e.g. afutuzumab, lenalidomide, thalidomide, pomalidomide; CD40 inhibitors: e.g. dacetuzumab; Pro-apoptotic receptor agonists (PARAs): e.g. dulanermin; Heat Shock Protein (HSP) inhibitors: e.g. tanespimycin (17-allylamino-17-desmethoxygeldanamycin); Hedgehog antagonists: e.g. vismodegib; Proteasome inhibitors: e.g. bortezomib; PI3K inhibitors: e.g. pictilisib, dactolisib, buparlisib, taselisib, idelalisib, duvelisib, umbralisib; Phospholipase A2 inhibitors: e.g. anagrelide; BCL-2 inhibitors: e.g. venetoclax; Aromatase inhibitors: exemestane, letrozole, anastrozole, faslodex, tamoxifen; Topoisomerase I inhibitors: e.g. irinotecan, topotecan; Topoisomerase II inhibitors: e.g. etoposide, teniposide; mTOR inhibitors: e.g. temsirolimus, ridaforolimus, everolimus, sirolimus; Osteoclastic bone resorption inhibitors: e.g. zoledronic acid; CD33 Antibody Drug Conjugates: e.g. gemtuzumab ozogamicin; CD22 Antibody Drug Conjugates: e.g. inotuzumab ozogamicin; CD20 Antibody Drug Conjugates: e.g. ibritumomab tiuxetan; Somatostain analogs: e.g. octreotide; Interleukin-11 (IL-11): e.g. oprelvekin; Synthetic erythropoietin: e.g. darbepoetin alfa; Receptor Activator for Nuclear Factor κ B (RANK) inhibitors: e.g. denosumab; Thrombopoietin mimetic peptides: e.g. romiplostim; Cell growth stimulators: e.g. palifermin; Anti-Insulin-like Growth Factor-1 receptor (IGF-1R) antibodies: e.g. figitumumab; Anti-CSl antibodies: e.g. elotuzumab; CD52 antibodies: e.g. alemtuzumab; CTLA-4 inhibitors: e.g. tremelimumab, ipilimumab; PDI inhibitors: e.g. nivolumab, pembrolizumab, an immunoadhesin; e.g. pidilizumab, AMP-224; PDL1 inhibitors: e.g. MSB0010718C; YW243.55.S70, MPDL3280A; MEDI-4736, MSB-0010718C, or MDX-1105; LAG-3 inhibitors: e.g. BMS-986016; GITR agonists; GITR fusion proteins and anti-GITR antibodies; Histone deacetylase inhibitors (HDI): e.g. voninostat; Anti-CTLA4 antibodies: e.g. tremelimumab, ipilimumab; Alkylating agents: e.g. temozolomide, dactinomycin, melphalan, altretamine carmustine, bendamustine, busulfan, carboplatin, lomustine, cisplatin, chlorambucil, cyclophosphamide, dacarbazine, altretamine, ifosfamide, procarbazine, mechlorethamine, mustine and mechloroethamine, streptozocin, thiotepa; Biologic response modifiers: e.g. bacillus calmette-guerin, denileukin diftitox; Anti-tumor antibiotics: e.g. doxorubicin, bleomycin, daunorubicin, daunorubicin liposomal, mitoxantrone, epirubicin, idarubicin, mitomycin C; Anti-microtubule agents: e.g. estramustine, Cathepsin K inhibitors: e.g. odanacatib; Epothilone analogs: e.g. ixabepilone; TpoR agonists: e.g. eltrombopag; Anti-mitotic agents: e.g. docetaxel; Adrenal steroid inhibitors: e.g. aminoglutethimide; Anti-androgens: e.g. nilutamide; Androgen Receptor inhibitors: e.g. enzalutamide, abiraterone acetate, orteronel, galeterone, and seviteronel, bicalutamide, flutamide; Androgens: e.g. fluoxymesterone; CDK1 inhibitors: e.g. alvocidib, palbociclib, ribociclib, trilaciclib, abemaciclib; Gonadotropin-releasing hormone (GnRH) receptor agonists: e.g. leuprolide or leuprolide acetate; Taxane anti-neoplastic agents: e.g. cabazitaxel, larotaxel; 5-HTla receptor agonists: e.g. xaliproden; HPV vaccines: e.g. Cervarix® sold by GlaxoSmithKline, Gardasil® sold by Merck; Iron Chelating agents: e.g. deferasirox; Anti-metabolites: e.g. claribine, 5-fluorouracil, 6-thioguanine, pemetrexed, cytarabine, cytarabine liposomal, decitabine, hydroxyurea, fludarabine, floxuridine, cladribine, methotrexate, pentostatin; Bisphosphonates: e.g. pamidronate; Demethylating agents: e.g. 5-azacitidine, decitabine; Anti-tumor Plant Alkaloids: e.g. paclitaxel protein-bound; vinblastine, vincristine, vinorelbine, paclitaxel; Retinoids: e.g. alitretinoin, tretinoin, isotretinoin, bexarotene; Glucocorticosteroids: e.g. hydrocortisone, dexamethasone, prednisolone, prednisone, methylprednisolone; Cytokines: e.g. interleukin-2, interleukin-11 (oprevelkin), alpha interferon alfa (IFN-alpha); estrogen receptor downregulators: fulvestrant; Anti-estrogens: e.g. tamoxifen, toremifene; Selective estrogen receptor modulators (SERMs): e.g. raloxifene; Luteinizing hormone releasing hormone (LHRH) agonists: e.g. goserelin; Progesterones: e.g. megestrol; cytotoxic agents: arsenic trioxide, asparaginase (also known as L-asparaginase, Erwinia L-asparaginase; Anti-nausea drugs: e.g. NK-1 receptor antagonists (e.g. casopitant); Cytoprotective agents: e.g. amifostine, leucovorin; and Immune checkpoint inhibitors. The term “immune checkpoints” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta.

In some embodiments, the one or more additional therapeutic agent is selected from the following agents: anti-CDK4 antibodies; cytotoxic agents; Estrogen Receptor-targeted or other endocrine therapies, immune-checkpoint inhibitors, other CDK inhibitors, Receptor Tyrosine Kinase inhibitors, BRAF inhibitors, MEK inhibitors, PI3K inhibitors, SHP2 inhibitors, and SRC inhibitors. (See M. Katoh, Nat. Rev. Clin. Oncol. 2019, 16:105-122; Y. K. Chae, et al. Oncotarget 2017, 8:16052-16074; L. Formisano et al., Nat. Comm. 2019, 10:1373-1386; and references cited therein.)

The structure of the active compounds identified by code numbers, generic or trade names may be taken from the actual edition of the standard compendium “The Merck Index” or from databases, e.g. Patents International (e.g. IMS World Publications).

A compound described herein may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound described herein can be administered alone or in combination with one or more other therapeutic compounds, possible combination therapy taking the form of fixed combinations or the administration of a compound described herein and one or more other therapeutic compounds being staggered or given independently of one another, or the combined administration of fixed combinations and one or more other therapeutic compounds. A compound described herein can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from a provided compound-containing composition, as part of a multiple dosage regimen. Alternatively, those agents may be part of a single dosage form, mixed together with a compound described herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound described herein may be administered with another therapeutic agent simultaneously or sequentially in separate unit dosage forms or together in a single unit dosage form. Accordingly, the present disclosure provides a single unit dosage form comprising a compound described herein, an additional therapeutic agent, and a pharmaceutically acceptable carrier, adjuvant, or vehicle.

The amount of both a compound described herein and additional therapeutic agent (in those compositions which comprise an additional therapeutic agent as described above) that may be combined with the carrier materials to produce a single dosage form will vary depending upon the host treated and the particular mode of administration. Preferably, compositions described herein should be formulated so that a dosage of between 0.01-100 mg/kg body weight/day of a compound described herein can be administered.

In those compositions which comprise an additional therapeutic agent, that additional therapeutic agent and the compound described herein may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions described herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds described herein, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. The present disclosure also contemplates implantable devices coated with a compound described herein.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds described herein, the following general methods, and other methods known to one of ordinary skill in the art, can be applied to other classes and subclasses and species of each of these compounds, as described herein. Additional compounds described herein were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. In the general schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). The starting materials for the examples are either commercially available or are readily prepared by standard methods from known materials.

At least some of the compounds identified as “Intermediates” herein are contemplated as compounds of the disclosure.

### 4. Enumerated Embodiments

The following numbered embodiments, while non-limiting, are exemplary of certain aspects of the present disclosure:
1. A compound of formula I:

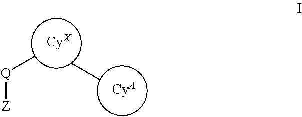

or a pharmaceutically acceptable salt thereof, wherein:
Q is L¹;
Z is H or Cy^{B};
Cy^{A} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{A} is substituted with m instances of R^{A};
Cy^{B} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, wherein Cy^{B} is substituted with n instances of R^{B}; Cy^{X} is

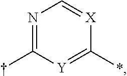

wherein [symbol] represents a bond to Q, and [symbol] represents a bond to Cy^{A};
X is N or CL²-X⁰;
X⁰ is hydrogen, a halogen, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{X};
Y is N or CH;
each instance of R^{A}, R^{B}, and R¹ is independently R¹ or R², wherein R^{A} is substituted by q^{A} instances of R³, R^{B} is substituted by q^{B} instances of R³, and R^{X} is substituted with q^{X} instances of R³; or
two instances of R^{A}, two instances of R^{B}, two instances of R^{X}, or an instance of R^{A} and an instance of R^{X} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; —O—C₁₋₇ aliphatic; C₁₋₄ haloalkyl; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{X}, r, and t is independently 0, 1, 2, 3, or 4.
2. The compound of embodiment 1, wherein Z is Cy^{B}.
3. The compound of embodiment 1, wherein Z is hydrogen.
4. The compound of any one of embodiments 1 to 3, wherein Q is a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, —NH—, —N(R^{L})—, or —O—.
5. The compound of any one of embodiments 1 to 4, wherein Q is —NH—,

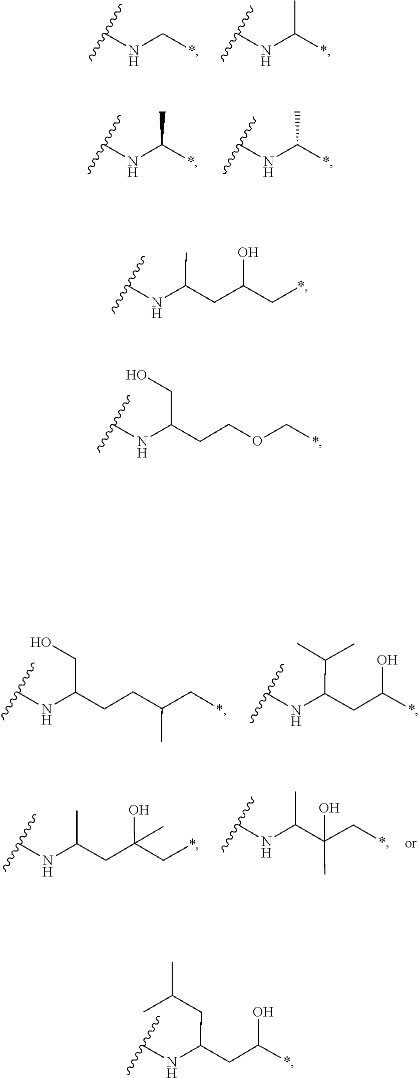

wherein

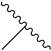

represents a covalent bond to Cy^{X} and [symbol] represents a covalent bond to Z.
6. The compound of embodiment 1, wherein the compound is of formula I-A:

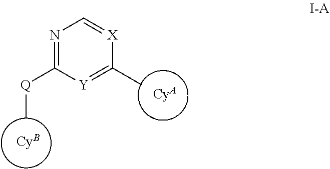

or a pharmaceutically acceptable salt thereof.
7. The compound of any one of embodiments 1-6, wherein Y is N.
8. The compound of any one of embodiments 1-6, wherein Y is CH.
9. The compound of any one of embodiments 1-8, wherein X is-CL²-X⁰.
10. The compound of any one of embodiments 1-9, wherein L² is a covalent bond.
11. The compound of embodiment 1, wherein the compound is of formula II, III, or IV:

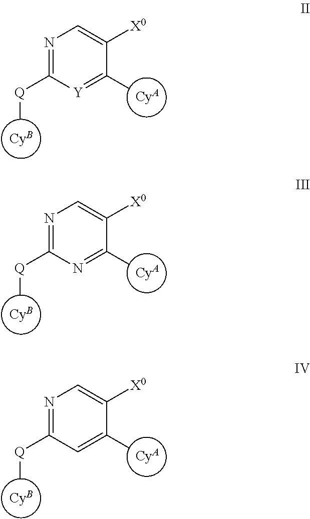

or a pharmaceutically acceptable salt thereof.
12. The compound of any one of embodiments 1-11, wherein Cy^{A} is an 8-10 membered bicyclic heteroaryl ring having 1-4 heteroatoms independently selected from nitrogen, oxygen, and sulfur.
13. The compound of any one of embodiments 1-11, wherein Cy^{A} is

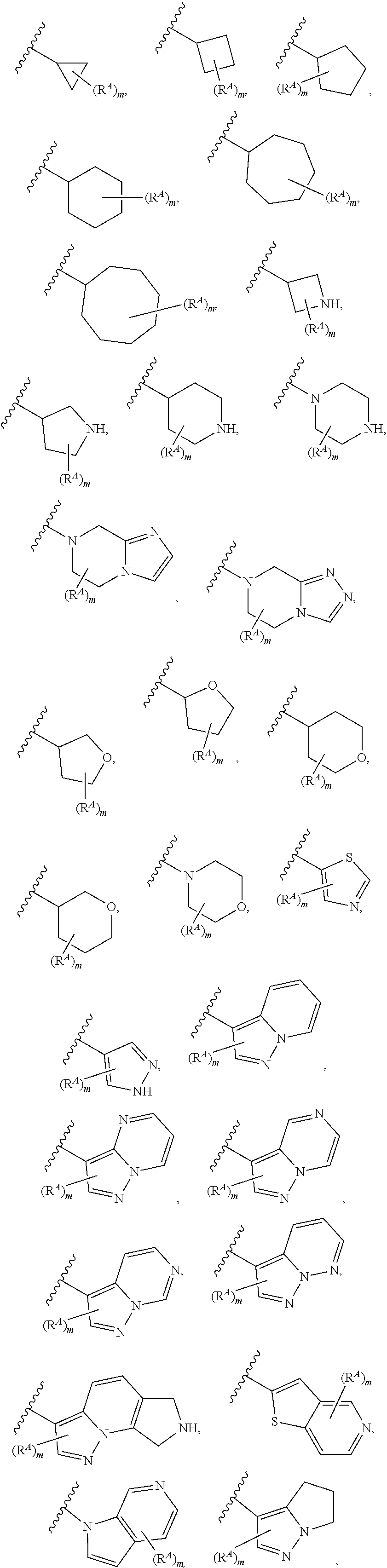

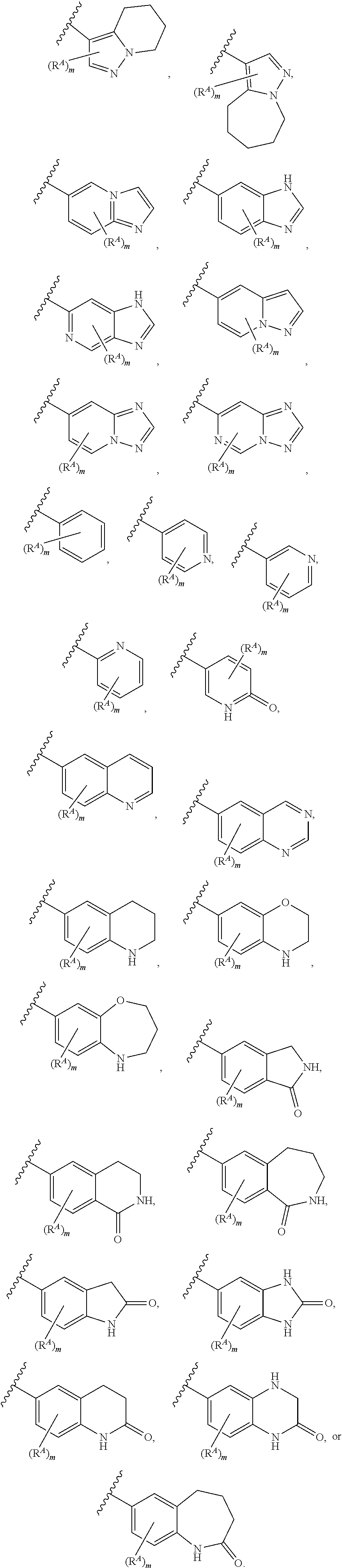

14. The compound of embodiment 1, wherein the compound is of formula II-A, II-B, II-C, II-D, II-E, II-F, II-G, II-H, II-II, II-J, II-K, II-L, II-M, II-N, II-O, II-P, II-Q, II-R, II-S, II-T, II-U, II-V, II-W, II-X, II-Y, II-Z, I-AA, I-BB, II-CC, I-DD, II-EE, II-FF, II-GG, II-HH, II-II, II-JJ, II-KK, II-LL, II-MM, II-NN, II-OO, II-PP, II-QQ, II-RR, II-SS, II-TT, II-UU, or II-VV:

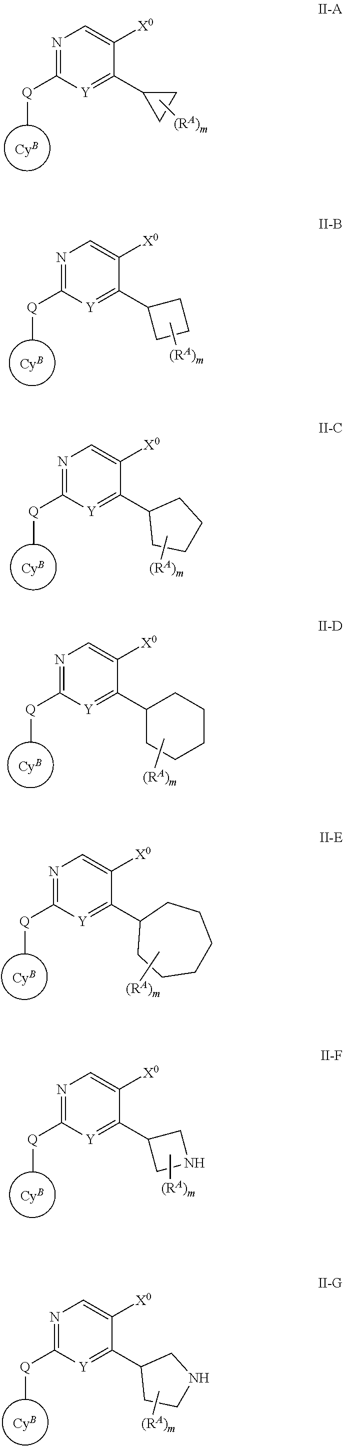

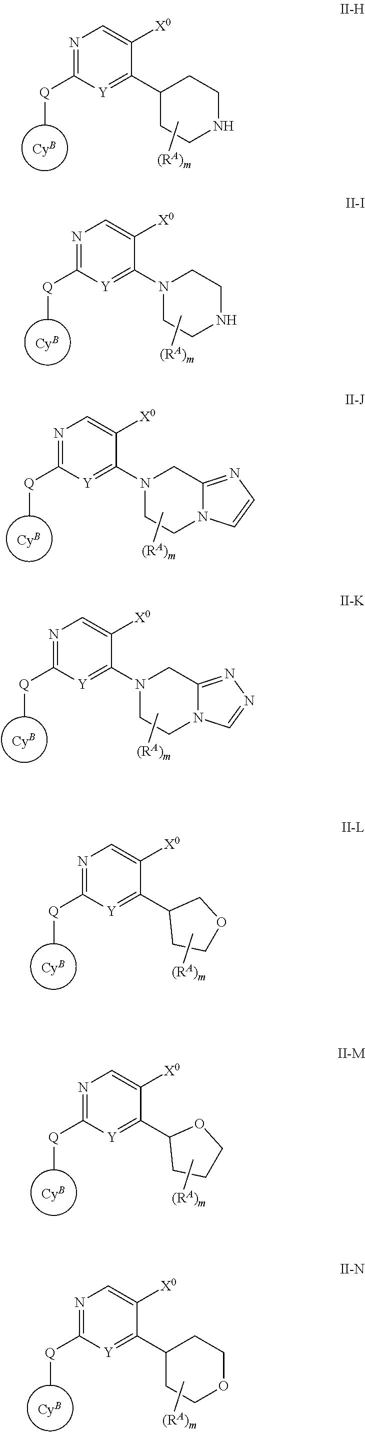

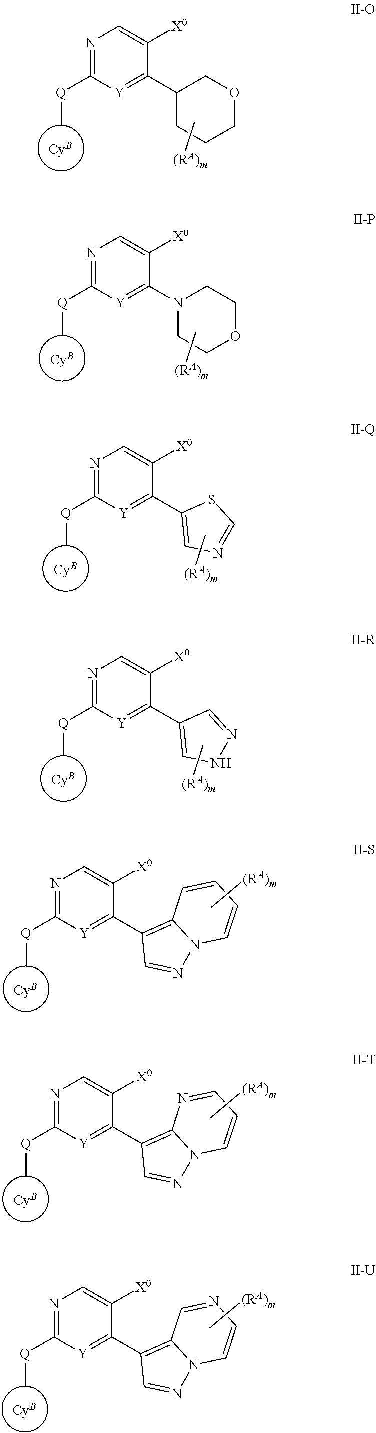

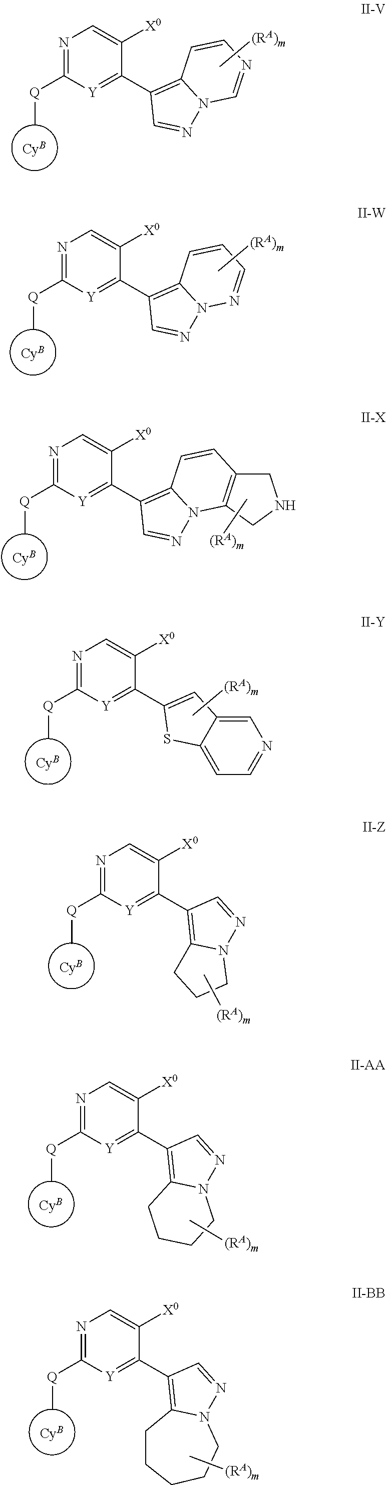

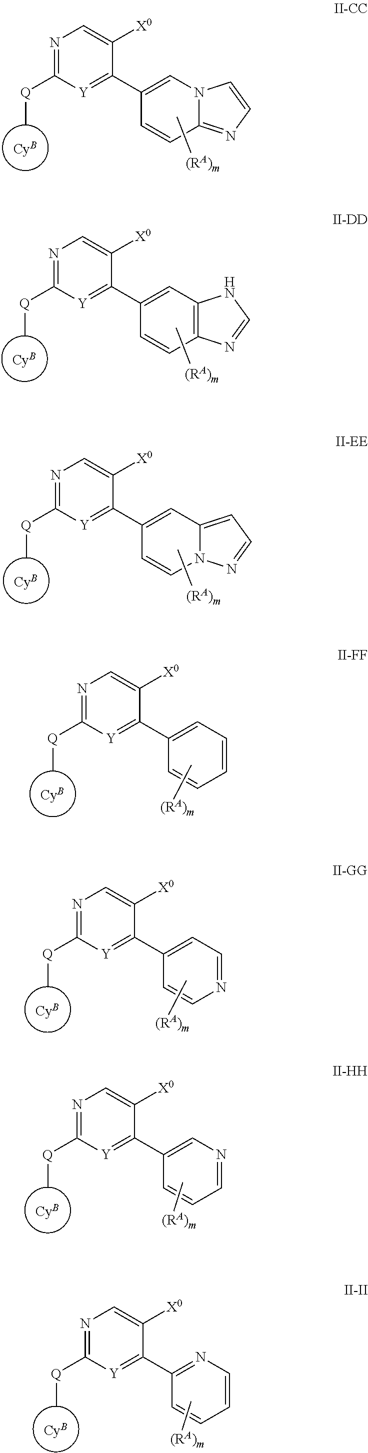

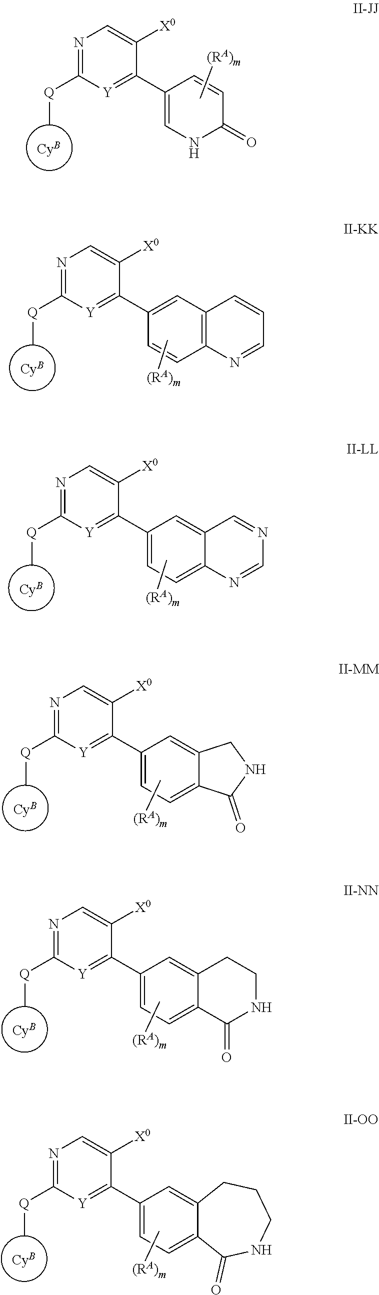

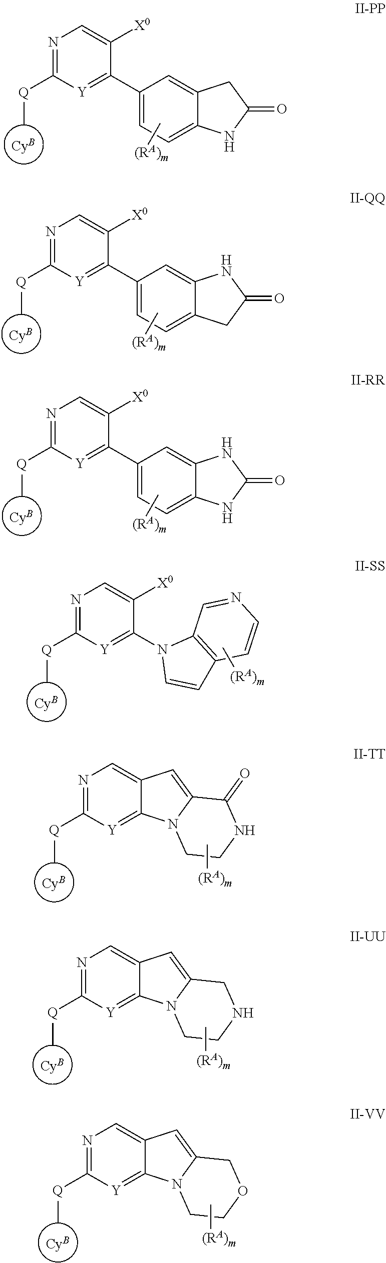

or a pharmaceutically acceptable salt thereof.
15. The compound of any one of embodiments 1-14, wherein Cy^{B} is a 3-8 membered saturated carbocyclic ring, wherein said ring is substituted with n instances of R^{B}.
16. The compound of any one of embodiments 1-14, wherein Cy^{B} is a 5-6 membered monocyclic heteroaryl ring having 1-4 heteroatoms independently selected from nitrogen, oxygen, and sulfur, wherein said ring is substituted with n instances of R^{B}.
17. The compound of embodiment 1, wherein the compound is of formula II-A1, II-B1, II-C1, II-D1, II-E1, II-F1, II-G1, II-H1, II-I1, II-J1, II-K1, II-L1, II-M1, II-N1, II-O1, II-P1, or II-Q1:

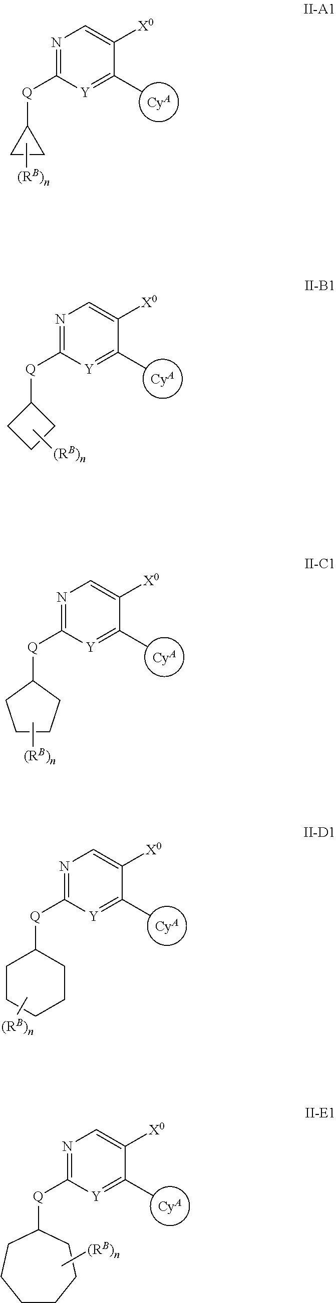

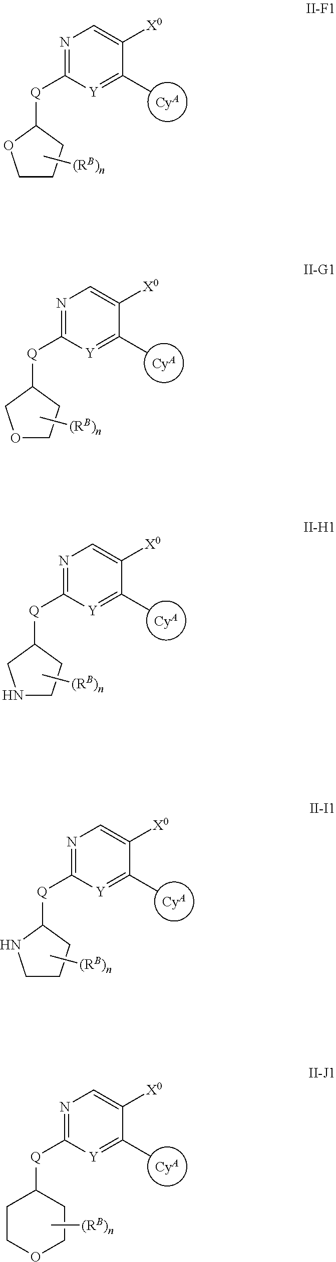

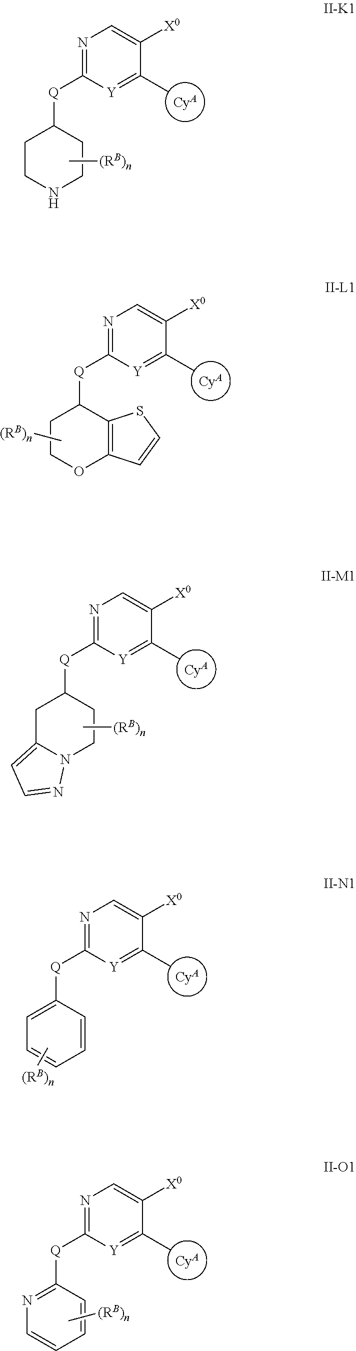

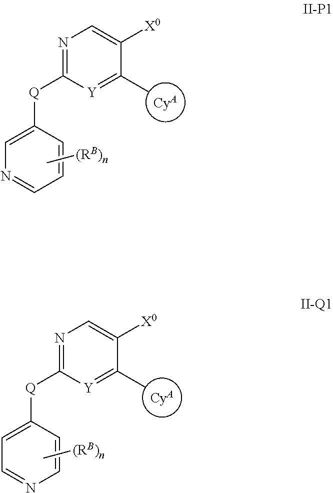

or a pharmaceutically acceptable salt thereof.
18. The compound of embodiment 1, wherein the compound is of formula II-A2, II-B2, II-C2, II-D2, II-E2, II-F2, II-G2, or II-H2:

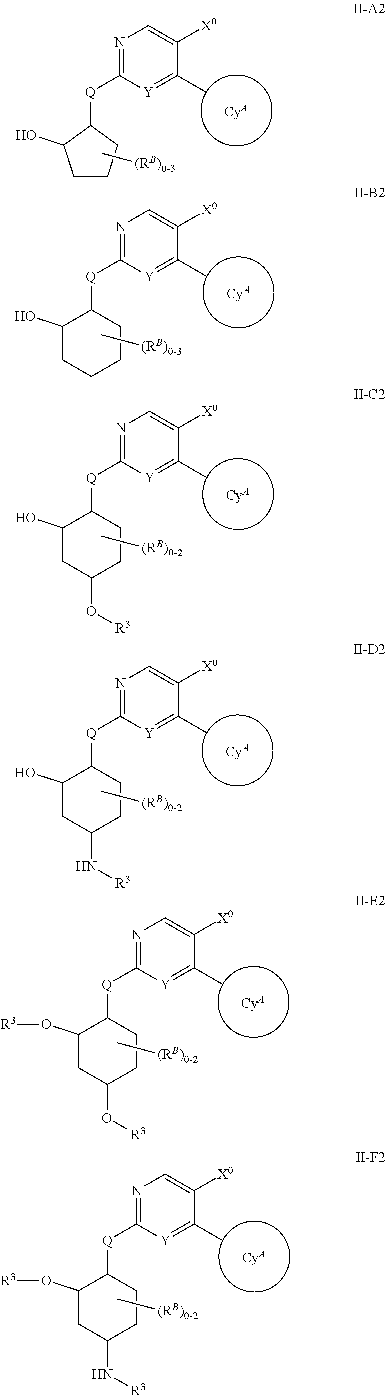

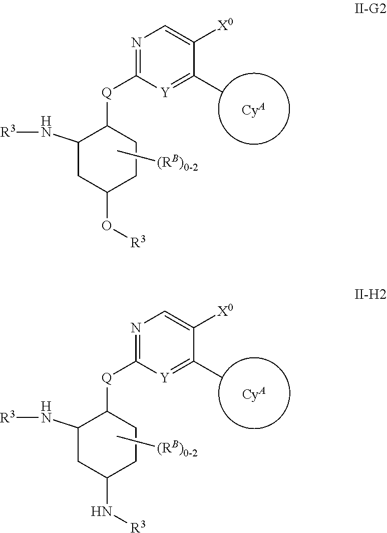

or a pharmaceutically acceptable salt thereof.
19. The compound of embodiment 1, wherein the compound is of formula V, VI, VII, VIII, IX, X, XI, XII, XIII, XIV, XV, XVI, XVII, XVIII, XIX, XX, XXI, or XXII:

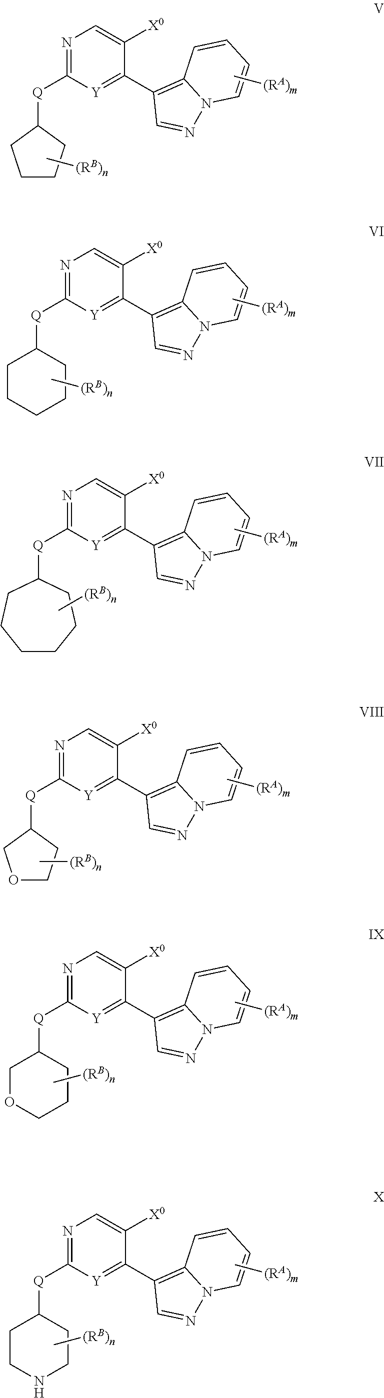

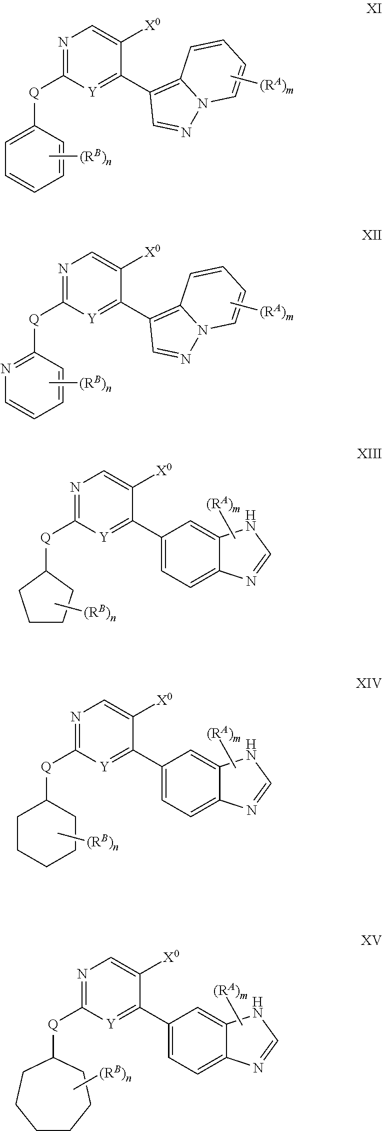

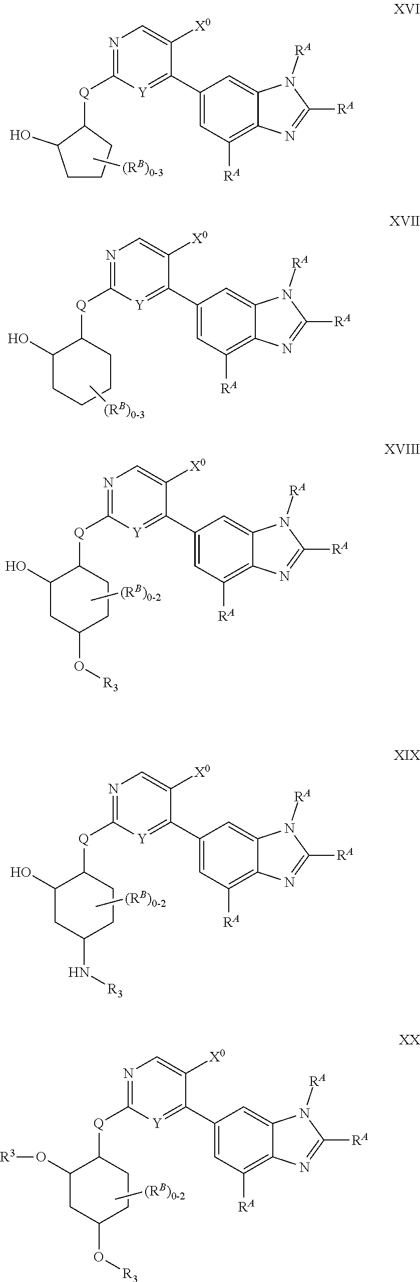

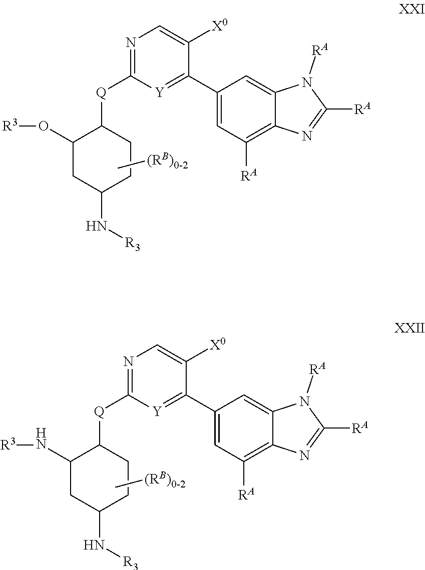

or a pharmaceutically acceptable salt thereof.
20. The compound of any one of embodiments 1-19, wherein X⁰ is halogen.
21. The compound of embodiment 20, wherein X⁰ is Cl.
22. The compound of embodiment 20, wherein X⁰ is F.
23. The compound of any one of embodiments 1-22, wherein Q is —NH—.
24. The compound of any one of embodiments 1-23, wherein at least one instance of R^{B} is oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.
25. The compound of any one of embodiments 1-23, wherein at least one instance of R^{B} is —OH.
26. The compound of any one of embodiments 1-25, wherein at least one instance of R^{A} is oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.
27. The compound of embodiment 26, wherein at least one instance of R^{A} is halogen.
28. The compound of any one of embodiments 1-25, wherein at least one instance of R^{A} is C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.
29. The compound of embodiment 28, wherein at least one instance of R^{A} is C₁₋₇ aliphatic substituted with q^{A} instances of R³.
30. The compound of any one of embodiment 1-29, wherein at least one instance of R³ is oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.
31. The compound of any one of embodiment 1-30, wherein at least one instance of R³ is halogen.
32. The compound of any one of embodiments 1-30, wherein at least one instance of R³ is —S(O)₂R, —S(O)₂NR₂, —C(O)OR, or —C(O)NR₂.
33. The compound of any one of embodiments 1-29, wherein at least one instance of R³ is an optionally substituted C₁₋₆ aliphatic.
34. The compound of embodiment 33, wherein at least one instance of R³ is methyl.
35. The compound of any one of embodiments 1-15, wherein the compound is selected from those depicted in Table 1, or a pharmaceutically acceptable salt thereof.
36. A pharmaceutical composition, comprising a compound of any one of embodiments 1-35, and a pharmaceutically acceptable carrier.
37. A method of inhibiting CDK4 signaling activity in a subject, comprising administering a therapeutically effective amount of a compound of any one of embodiments 1-35, or the pharmaceutical composition of embodiment 36, to a subject in need thereof.
38. A method of treating an CDK4-mediated disorder in a subject, comprising administering a therapeutically effective amount of a compound of any one of embodiments 1-35, or the pharmaceutical composition of embodiment 36, to a subject in need thereof.
39. A method of treating a disorder in a subject, wherein the disorder is ovarian cancer, endometrial cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, or pancreatic cancer, the method comprising administering to the subject a therapeutically effective amount of a compound of any one of embodiments 1-35, or the pharmaceutical composition of embodiment 36, to a subject in need thereof.
40. The method of embodiment 39, wherein the bladder cancer is urothelial carcinoma.
41. The method of embodiment 39, wherein the liver cancer is hepatocellular carcinoma.
42. The method of embodiment 39, wherein the lung cancer is lung squamous cell carcinoma or non-small cell lung cancer.
43. The method of embodiment 39, wherein the laryngeal cancer is laryngeal squamous cell carcinoma.
44. The method of embodiment 39, wherein the skin cancer is melanoma.

### Example 1

### (4-(cyclopropylamino)piperidin-1-yl)(6-((5-fluoro-4-(4-fluoro-1-isopropyl-2-methyl-1H-benzo[d]imidazol-6-yl)pyrimidin-2-yl)amino)-2-methylpyridin-3-yl)methanone (Compound I-17)

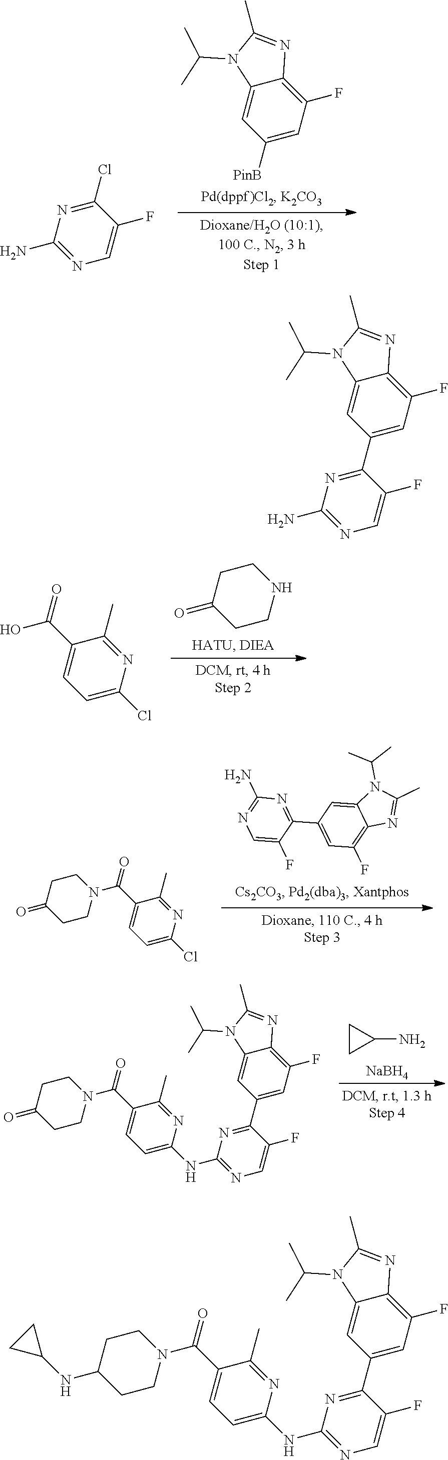

### 5-fluoro-4-(4-fluoro-1-isopropyl-2-methyl-1H-benzo[d]imidazol-6-yl)pyrimidin-2-amine

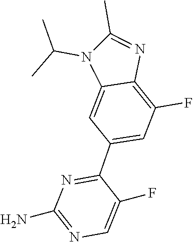 [SMILES: Cc1nc2c(F)cc(-c3nc(N)ncc3F)cc2n1C(C)C]

Step 1: 4-fluoro-1-isopropyl-2-methyl-6-(4,4,5,5-tetramethyl-1,3,2-dioxaborolan-2-yl)-1H-benzo[d]imidazole (2 g, 1 eq., 6 mmol), Pd(dppf)Cl₂ (0.5 g, 0.1 eq., 0.6 mmol) and K₂CO₃ (2 g, 2 eq., 0.01 mol) were added to a solution of 4-chloro-5-fluoropyrimidin-2-amine (465 mg, 0.5 eq., 3.15 mmol) in dioxane/H₂O (10:1, 10 mL) under nitrogen atmosphere. The reaction mixture was heated at 100° C. for 3 hours with vigorous stirring and then quenched with water. The precipitated solids were collected by filtration and washed with EtOAc (20 mL) to yield 5-fluoro-4-(4-fluoro-1-isopropyl-2-methyl-1H-benzo[d]imidazol-6-yl) pyrimidin-2-amine (1.4 g, 4.6 mmol, 70%) as a yellow solid. m/z (ES⁺) [M+H]⁺=304.20; HPLC tR=0.665 min.

### 1-(6-chloro-2-methylnicotinoyl)piperidin-4-one

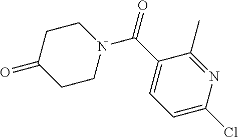 [SMILES: Cc1nc(Cl)ccc1C(=O)N1CCC(=O)CC1]

Step 2: To a solution of 6-chloro-2-methylnicotinic acid (1 g, 1 eq., 6 mmol) in DCM (50 mL) were added piperidin-4-one (0.9 g, 1.5 eq., 9 mmol), DIEA (2 mL, 12M, 4 eq., 0.02 mol), and HATU (2.2 g, 1 eq., 5.8 mmol). The mixture was stirred at 25° C. for 4 hours, and then concentrated in vacuo. The residue was purified by reverse-phase flash chromatography (column: C18 silica gel; mobile phase: ACN in water; gradient: 10% to 70% in 15 min; wavelength: 254 nm). The desired fractions were combined, and then concentrated in vacuo to afford 1-(6-chloro-2-methylnicotinoyl) piperidin-4-one (900 mg, 3.56 mmol, 60%) as a colorless liquid. m/z (ES⁺) [M+H]⁺=253.15; HPLC tR=0.648 min.

### 1-(6-((5-fluoro-4-(4-fluoro-1-isopropyl-2-methyl-1H-benzo[d]imidazol-6-yl) pyrimidin-2-yl) amino)-2-methylnicotinoyl) piperidin-4-one

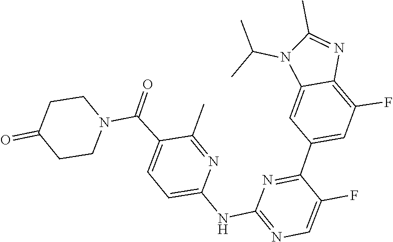 [SMILES: Cc1nc(Nc2ncc(F)c(-c3cc(F)c4nc(C)n(C(C)C)c4c3)n2)ccc1C(=O)N1CCC(=O)CC1]

Step 3: 5-fluoro-4-(4-fluoro-1-isopropyl-2-methyl-1H-benzo[d]imidazol-6-yl) pyrimidin-2-amine (401 mg, 1 eq., 1.32 mmol), Cs₂CO₃ (859 mg, 2 eq., 2.64 mmol), Xanphos (140 mg, 0.183 eq., 242 μmol) and Pd₂(dba)₃ (100 mg, 0.0826 eq., 109 μmol) were added to a solution of 1-(6-chloro-2-methylnicotinoyl) piperidin-4-one (334 mg, 1 eq., 1.32 mmol) in 1,4-dioxane (10 mL) under nitrogen atmosphere. The reaction mixture was heated at 110° C. for 4 hours with vigorous stirring and then concentrated in vacuo. The residue was purified by reverse-phase flash chromatography (column: C18 silica gel; mobile phase: ACN in water; gradient: 10% to 70% in 15 min; wavelength: 254 nm). The desired fractions were combined, and then concentrated in vacuo to afford 1-(6-((5-fluoro-4-(4-fluoro-1-isopropyl-2-methyl-1H-benzo[d]imidazol-6-yl) pyrimidin-2-yl) amino)-2-methylnicotinoyl) piperidin-4-one (160 mg, 308 mol, 23.3%) as a yellow solid. m/z (ES⁺) [M+H]⁺=520.45; HPLC tR=0.919 min.

### (4-(cyclopropylamino)piperidin-1-yl)(6-((5-fluoro-4-(4-fluoro-1-isopropyl-2-methyl-1H-benzo[d]imidazol-6-yl)pyrimidin-2-yl)amino)-2-methylpyridin-3-yl)methanone (Compound I-17)

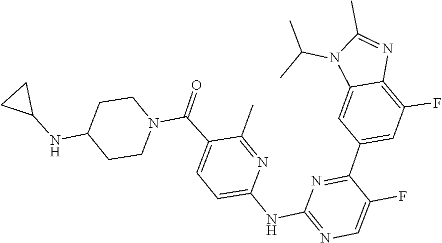 [SMILES: Cc1nc(Nc2ncc(F)c(-c3cc(F)c4nc(C)n(C(C)C)c4c3)n2)ccc1C(=O)N1CCC(NC2CC2)CC1]

Step 4: To a solution of 1-(6-((5-fluoro-4-(4-fluoro-1-isopropyl-2-methyl-1H-benzo[d]imidazol-6-yl)pyrimidin-2-yl)amino)-2-methylnicotinoyl)piperidin-4-one (160 mg, 1 eq., 308 μmol) in DCM (9 mL) was added cyclopropanamine (0.33 g, 0.48 mL, 12M, 19 eq., 5.8 mmol) at 25° C., and the resulting mixture was stirred at 25° C. for 30 min. Then, sodium borohydride (0.34 g, 30 eq., 9.24 mmol) was added to the mixture, which was stirred at 25° C. for 1.3 hours and then concentrated in vacuo. The resulting crude material was purified by preparative HPLC (column: XBridge Prep OBD C18 column, 30×150 mm, 5 μm; mobile phase A: water (10 mM NH₄HCO₃+0.1% NH₃·H₂O); mobile phase B: ACN; flow rate: 60 mL/min; gradient: 28% B to 50% B in 8 min, 50% B; wavelength: 220 nm; tR1 (min): 8.08;). The desired fractions were combined, and then lyophilized to afford (4-(cyclopropylamino)piperidin-1-yl)(6-((5-fluoro-4-(4-fluoro-1-isopropyl-2-methyl-1H-benzo[d]imidazol-6-yl)pyrimidin-2-yl)amino)-2-methylpyridin-3-yl)methanone (27.7 mg, 49.4 μmol, 16.0%) as a white amorphous solid. m/z (ES⁺) [M+H]⁺=561.25; HPLC tR=0.537 min. ¹H NMR (400 MHz, DMSO-d₆) 10.23 (s, 1H), 8.72 (d, J=3.8 Hz, 1H), 8.32 (d, J=1.3 Hz, 1H), 8.15 (d, J=8.5 Hz, 1H), 7.71 (dd, J=12.0, 1.3 Hz, 1H), 7.56 (d, J=8.5 Hz, 1H), 4.84 (h, J=7.0 Hz, 1H), 4.32 (d, J=12.9 Hz, 1H), 3.41 (d, J=13.4 Hz, 1H), 3.33 (s, 1H), 3.03 (dt, J=23.5, 12.1 Hz, 2H), 2.80 (s, 1H), 2.65 (s, 3H), 2.36 (s, 3H), 2.10 (s, 1H), 1.94 (d, J=12.1 Hz, 1H), 1.79 (d, J=9.7 Hz, 1H), 1.64 (d, J=6.9 Hz, 6H), 1.37-1.05 (m, 2H), 0.39 (d, J=6.2 Hz, 2H), 0.25-0.21 (m, 2H).

### Example 2

### (S)—N-(5-(3-(dimethylamino)pyrrolidin-1-yl)pyridin-2-yl)-5-fluoro-4-(4-fluoro-1-isopropyl-2-methyl-1H-benzo[d]imidazol-6-yl)pyrimidin-2-amine (Compound I-21)

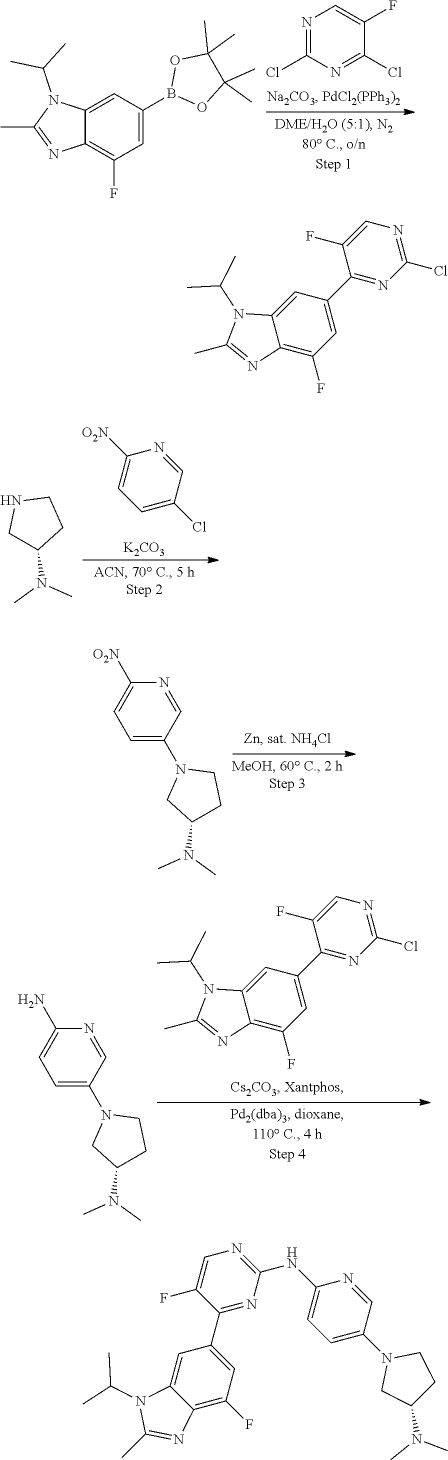

### 6-(2-chloro-5-fluoropyrimidin-4-yl)-4-fluoro-1-isopropyl-2-methyl-1H-benzo[d]imidazole

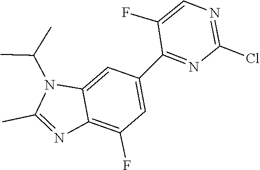 [SMILES: Cc1nc2c(F)cc(-c3nc(Cl)ncc3F)cc2n1C(C)C]

Step 1: To a mixture of 4-fluoro-1-isopropyl-2-methyl-6-(4,4,5,5-tetramethyl-1,3,2-dioxaborolan-2-yl)-1H-benzo[d]imidazole (1.00 g, 3.00 mmol), 2,4-dichloro-5-fluoropyrimidine (0.80 g, 5.00 mmol) and Na₂CO₃ (1.00 g, 9.00 mmol) in DME:H₂O=5:1 (12 mL) was added bis-(triphenylphosphine)palladium chloride (0.20 g, 0.30 mmol) under nitrogen atmosphere. The mixture was stirred at 80° C. for 16 hours. The reaction mixture was diluted with water (20 mL), and the aq. phase was extracted with EtOAc (3×20 mL). The combined organic layers were washed with brine, dried over sodium sulfate, filtered, and concentrated in vacuo. The crude product was re-crystallized. The precipitated solids were collected by filtration and washed with EtOAc to obtain 6-(2-chloro-5-fluoropyrimidin-4-yl)-4-fluoro-1-isopropyl-2-methyl-1H-benzo[d]imidazole (730 mg, 70%) as a yellow amorphous solid. m/z (ES⁺) [M+H]⁺=323.05; HPLC tR=1.098 min.

### (S)—N,N-dimethyl-1-(6-nitropyridin-3-yl)pyrrolidin-3-amine

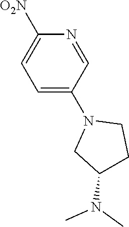 [SMILES: CN(C)[C@H]1CCN(c2ccc([N+](=O)[O-])nc2)C1]

Step 2: A round bottomed flask was charged with (S)—N,N-dimethylpyrrolidin-3-amine (500 mg, 4.38 mmol), 5-chloro-2-nitropyridine (1.04 g, 6.57 mmol), K₂CO₃ (2.42 g, 17.50 mmol), a stir bar, and ACN (10 mL), and then the solution was stirred for 5 hour at 70° C. The reaction mixture was concentrated in vacuo. The residue was purified by reverse-phase flash chromatography (column: C18 silica gel; mobile phase, ACN in water, 10% to 70% gradient in 15 min; wavelength: 254 nm. The desired fractions were combined, and then concentrated in vacuo to afford (S)—N,N-dimethyl-1-(6-nitropyridin-3-yl)pyrrolidin-3-amine (600 mg, 58.0%) as a yellow oil. m/z (ES⁺) [M+H]⁺=237.15; HPLC tR=0.417 min.

### (S)-5-(3-(dimethylamino)pyrrolidin-1-yl)pyridin-2-amine

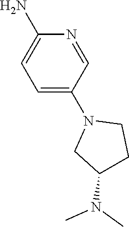

Step 3: A resealable reaction vial was charged with (S)—N,N-dimethyl-1-(6-nitropyridin-3-yl)pyrrolidin-3-amine (400 mg, 1.69 mmol), zinc (443 mg, 6.77 mmol), sat'd. aq. NH₄Cl (2 mL), MeOH (6 mL) and a stir bar, and then evacuated and purged with nitrogen three times. The mixture was stirred at 60° C. for 2 hours. The reaction mixture was filtered, the solid was washed with MeOH, and the filtrate was concentrated in vacuo. The residue was purified by reverse-phase flash chromatography (column: C18 silica gel; mobile phase, ACN in water, 10% to 80% gradient in 20 min; wavelength: 254 nm). The desired fractions were combined, and then concentrated in vacuo to afford (S)-5-(3-(dimethylamino)pyrrolidin-1-yl)pyridin-2-amine (200 mg, 57.3%) as a pink amorphous solid. m/z (ES⁺) [M+H]⁺=207.00; HPLC tR=0.495 min.

### (S)—N-(5-(3-(dimethylamino)pyrrolidin-1-yl)pyridin-2-yl)-5-fluoro-4-(4-fluoro-1-isopropyl-2-methyl-1H-benzo[d]imidazol-6-yl)pyrimidin-2-amine (Compound I-21)

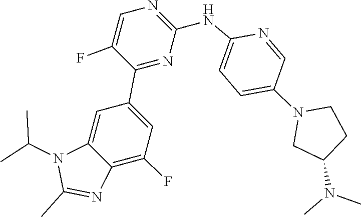 [SMILES: Cc1nc2c(F)cc(-c3nc(Nc4ccc(N5CC[C@H](N(C)C)C5)cn4)ncc3F)cc2n1C(C)C]

Step 4: A resealable reaction vial was charged with (S)-5-(3-(dimethylamino)pyrrolidin-1-yl)pyridin-2-amine (100 mg, 0.49 mmol), 6-(2-chloro-5-fluoropyrimidin-4-yl)-4-fluoro-1-isopropyl-2-methyl-1H-benzo[d]imidazole (156 mg, 0.49 mmol), 1,4-dioxane (6 mL), Cs₂CO₃ (316 mg, 0.97 mmol), Xantphos (28 mg, 0.045 mmol), Pd₂(dba)₃ (44 mg, 0.05 mmol) and a stir bar, and then evacuated and purged with nitrogen three times. The mixture was stirred at 110° C. for 4 hours and then concentrated in vacuo. The resulting crude material was purified by preparative HPLC (column: XBridge Shield RP18 OBD Column, 30×150 mm, 5 μm; mobile phase A: water (10 mM NH₄HCO₃); mobile phase B: ACN; flow rate: 60 mL/min; gradient: 32% B to 57% B in 8 min, 57% B; wavelength: 220 nm; tR1 (min): 7.45). The desired fractions were combined, and then lyophilized to afford (S)—N-(5-(3-(dimethylamino)pyrrolidin-1-yl)pyridin-2-yl)-5-fluoro-4-(4-fluoro-1-isopropyl-2-methyl-1H-benzo[d]imidazol-6-yl)pyrimidin-2-amine (55.8 mg, 23.4%) as a yellow amorphous solid. m/z (ES⁺) [M+H]⁺=493.35; HPLC tR=0.808 min. ¹H NMR (400 MHz, DMSO-d6) 9.57 (s, 1H), 8.59 (d, J=3.9 Hz, 1H), 8.26 (d, J=1.3 Hz, 1H), 7.96 (d, J=8.9 Hz, 1H), 7.73-7.63 (m, 2H), 7.02 (dd, J=9.0, 3.1 Hz, 1H), 4.83 (q, J=7.1 Hz, 2H), 3.45 (t, J=8.2 Hz, 1H), 3.38 (s, 1H), 3.26 (d, J=7.3 Hz, 1H), 3.06 (t, J=8.4 Hz, 1H), 2.86-2.79 (m, 1H), 2.64 (s, 3H), 2.21 (s, 6H), 2.16 (d, J=8.8 Hz, 1H), 1.81 (dd, J=21.1, 9.9 Hz, 1H), 1.62 (d, J=6.9 Hz, 6H).

### Example 3

### (1R,3R,4R)-4-((5-chloro-4-(4-fluoro-1-isopropyl-2-methyl-1H-benzo[d]imidazol-6-yl)pyrimidin-2-yl)amino)cyclohexane-1,3-diol (Compound I-34)

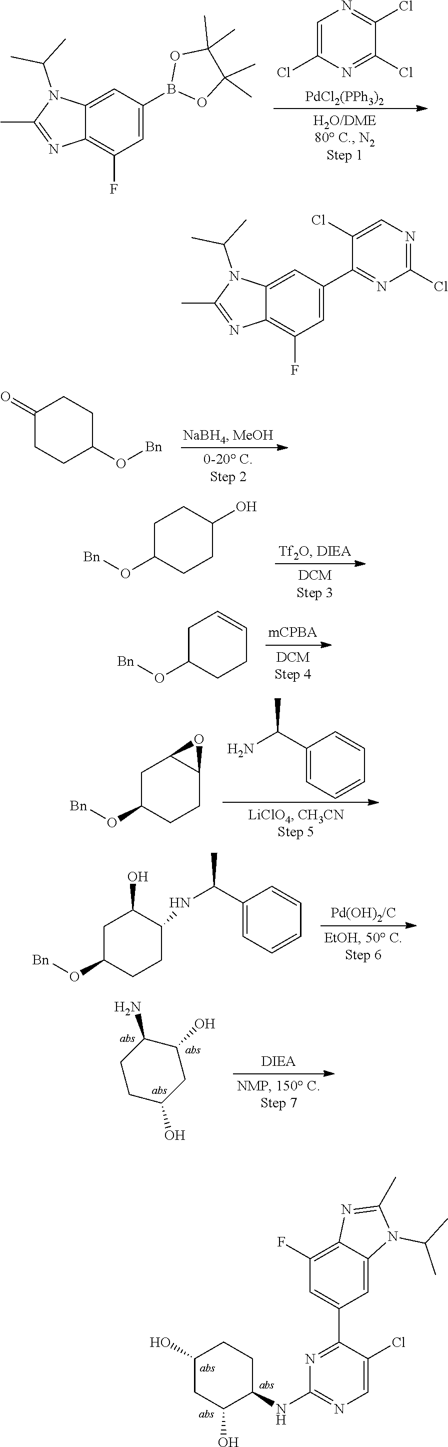

### 6-(2,5-dichloropyrimidin-4-yl)-4-fluoro-1-isopropyl-2-methyl-1H-benzo[d]imidazole

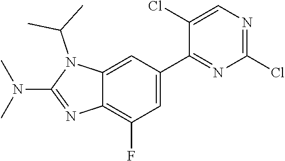 [SMILES: CC(C)n1c(N(C)C)nc2c(F)cc(-c3nc(Cl)ncc3Cl)cc21]

Step 1: A solution of 4-fluoro-1-isopropyl-2-methyl-6-(4,4,5,5-tetramethyl-1,3,2-dioxaborolan-2-yl)-1H-benzo[d]imidazole (3 g, 9 mmol), 2,4,5-trichloropyrimidine (2 g, 9 mmol), and sodium carbonate (3 g, 0.03 mol) in 1,2-dimethoxyethane and water was added to bis(triphenylphosphine)palladium(II) dichloride (0.3 g, 0.5 mmol). The reaction mixture was stirred at 80° C. under N₂ atmosphere for 15 hours. The precipitated solids were collected by filtration and washed with EtOAc (100 mL). This afforded 6-(2,5-dichloropyrimidin-4-yl)-4-fluoro-1-isopropyl-2-methyl-1H-benzo[d]imidazole (1.66 g, 50%) as a light yellow solid. m/z (ES⁺) [M+H]⁺=339.05; HPLC tR=0.759 min.

### 4-(benzyloxy)cyclohexan-1-ol

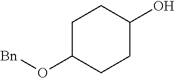 [SMILES: OC1CCC(OCc2ccccc2)CC1]

Step 2: To an ice-bath cooled solution of 4-(benzyloxy)cyclohexan-1-one (25 g, 0.12 mol) in MeOH (40 mL), was added NaBH₄ (5.6 g, 0.15 mol) in several portions during a period of 10 min, then the solution was stirred at 20° C. for 2 hours. The reaction mixture was diluted with H₂O (50 mL), and the aq. phase was extracted with EtOAc (3×100 mL). The combined organic layers were washed with brine, dried over sodium sulfate, filtered, and then concentrated in vacuo. The residue was purified by silica-gel chromatography, eluting with PE/EtOAc (30:1). The desired fractions were combined, and then concentrated in vacuo to afford 4-(benzyloxy)cyclohexan-1-ol (8 g, 32%) as a colorless oil. m/z (ES⁺) [M+H]⁺=207.05; HPLC tR=0.698 min.

### ((cyclohex-3-en-1-yloxy)methyl)benzene

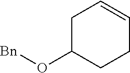 [SMILES: C1=CCC(OCc2ccccc2)CC1]

Step 3: To an ice-bath cooled solution of 4-(benzyloxy)cyclohexan-1-ol (10 g, 48 mmol) and DEA (9.4 g, 73 mmol) in DCM (250 mL), was added Tf₂O (10 g, 36 mmol) dropwise over 30 min and then the solution was stirred at 25° C. for 16 hours. Next, the mixture was concentrated under reduced pressure and the residue was purified with silica-gel chromatography, eluting with PE/EtOAc (50:1). The desired fractions were combined, and then concentrated in vacuo to afford ((cyclohex-3-en-1-yloxy)methyl)benzene (7.2 g, 79%) as a yellow oil. m/z (ES⁺) [M+ACN]⁺=230.20; HPLC tR=1.285 min.

### rac-(1R,3R,6S)-3-(benzyloxy)-7-oxabicyclo[4.1.0]heptane

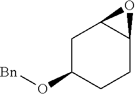 [SMILES: c1ccc(CO[C@@H]2CC[C@@H]3O[C@@H]3C2)cc1]

Step 4: To a solution of ((cyclohex-3-en-1-yloxy)methyl)benzene (7.2 g, 38 mmol) in DCM (50 mL) was added 3-chlorobenzoperoxoic acid (13 g, 76 mmol) at 0° C. and stirred for 2 hours. The reaction mixture was diluted with H₂O (50 mL), and the aq. phase was extracted with EtOAc (3×100 mL). The combined organic layers were washed with brine, dried over sodium sulfate, filtered, and then concentrated in vacuo. The residue was purified by silica-gel chromatography (15 g), eluting with PE/EtOAc 30:1 to afford the product as a mixture (4.2 g). The mixture was purified by flash chromatography, eluting with PE/EtOA (10:1). The desired fractions were combined, and then concentrated in vacuo to afford rac-(1R,3R,6S)-3-(benzyloxy)-7-oxabicyclo[4.1.0]heptane (1.1 g, 28%) as a colorless oil. m/z (ES⁺) [M+H]⁻=205.15; HPLC tR=1.061 min.

### (1R,2R,5R)-5-(benzyloxy)-2-(((S)-1-phenylethyl)amino)cyclohexan-1-ol

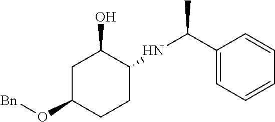 [SMILES: C[C@H](N[C@@H]1CC[C@@H](OCc2ccccc2)C[C@H]1O)c1ccccc1]

Step 5: A round-bottomed flask was charged with rac-(1R,3R,6S)-3-(benzyloxy)-7-oxabicyclo[4.1.0]heptane (400 mg, 1.96 mmol), 4A-MS (100 mg), lithium perchlorate (417 mg, 3.92 mmol) and (S)-1-phenylethan-1-amine (308 mg, 2.55 mmol) in ACN (3 mL) and a stir bar, the solution was stirred for 16 hour at 25° C. The reaction mixture was diluted with H₂O (50 mL), and the aq. phase was extracted with EtOAc (3×100 mL). The combined organic layers were washed with brine, dried over sodium sulfate, filtered, and then concentrated in vacuo. The resulting crude material was purified by preparative HPLC (column: XBridge Shield RP18 OBD Column, 30×150 mm, 5 μm; mobile phase A: water (10 mM NH₄HCO₃); mobile phase B: ACN; flow rate: 60 mL/min; gradient: 33% B to 58% B in 8 min, 58% B; wavelength: 220 nm & 254 nm; tR1 (min): 7.27; and column: XBridge Shield RP18 OBD Column, 30×150 mm, 5 μm). The desired fractions were combined, and then lyophilized to afford (1R,2R,5R)-5-(benzyloxy)-2-(((S)-1-phenylethyl)amino)cyclohexan-1-ol (100 mg, 50.0%) as a colorless oil. m/z (ES⁺) [M+H]⁻=326.20; HPLC tR=1.098 min.

### (1R,3R,4R)-4-aminocyclohexane-1,3-diol

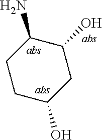

Step 6: A stirred mixture of (1R,2R,5R)-5-(benzyloxy)-2-(((S)-1-phenylethyl)amino)cyclohexan-1-ol (100 mg, 0.31 mmol) and Pd(OH)₂/C (43 mg, 0.31 mmol) in EtOH (8 mL) was treated with H₂ at 50° C. for 3 hours. The reaction mixture was filtered through a pad of Celite, the pad was washed with EtOH, and the filtrate was concentrated in vacuo. This afforded (1R,3R,4R)-4-aminocyclohexane-1,3-diol (50 mg, 87%) as a yellow oil. m/z (ES⁺) [M+H]⁺=132.10; HPLC tR=0.158 min.

### (1R,3R,4R)-4-((5-chloro-4-(4-fluoro-1-isopropyl-2-methyl-1H-benzo[d]536midazole-6-yl)pyrimidin-2-yl)amino)cyclohexane-1,3-diol (Compound I-34)

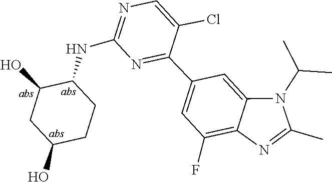 [SMILES: Cc1nc2c(F)cc(-c3nc(N[C@@H]4CC[C@@H](O)C[C@H]4O)ncc3Cl)cc2n1C(C)C]

Step 7: To a mixture of (1R,3R,4R)-4-aminocyclohexane-1,3-diol (60 mg, 0.46 mmol) and 6-(2,5-dichloropyrimidin-4-yl)-4-fluoro-1-isopropyl-2-methyl-1H-benzo[d]imidazole (0.16 g, 0.46 mmol) in NMP (6 mL) was added DIEA (0.18 g, 1.4 mmol). The mixture was stirred at 150° C. for 4 hours. The solution was purified by reverse-phase flash chromatography (column: C18 silica gel; mobile phase, ACN in water, 20% to 60% gradient in 15 min; wavelength: 254 nm). The desired fractions were combined, and then concentrated in vacuo to afford (1R,3R,4R)-4-((5-chloro-4-(4-fluoro-1-isopropyl-2-methyl-1H-benzo[d]imidazol-6-yl)pyrimidin-2-yl)amino)cyclohexane-1,3-diol (11 mg, 5.4%) as an off-white amorphous solid. m/z (ES⁺) [M+H]⁻=434.15; HPLC tR=1.492 min. ¹H NMR (400 MHz, DMSO-d6) 8.39 (s, 1H), 7.93 (s, 1H), 7.42 (s, 1H), 7.23 (s, 1H), 4.81 (p, J=6.9 Hz, 1H), 4.65 (t, J=6.4 Hz, 2H), 3.55 (s, 1H), 3.44 (s, 2H), 2.63 (s, 3H), 2.09 (d, J=11.6 Hz, 1H), 1.92 (s, 1H), 1.78 (s, 1H), 1.59 (d, J=6.9 Hz, 6H), 1.27-1.11 (m, 3H).

### Example 4

### 2′-(((3S,4R)-3-hydroxytetrahydro-2H-pyran-4-yl)amino)-7′,8′-dihydro-6′H-spiro[cyclohexane-1,9′-pyrazino[1′,2′:1,5]pyrrolo[2,3-d]pyrimidin]-6′-one (Compound I-22)

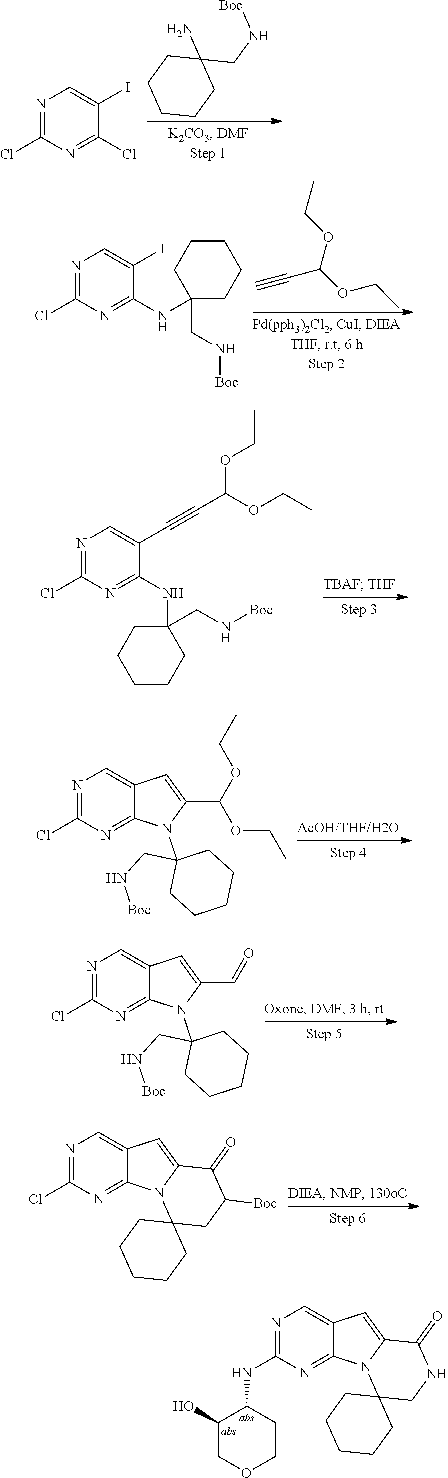

### tert-butyl ((1-((2-chloro-5-iodopyrimidin-4-yl)amino)cyclohexyl)methyl)carbamate

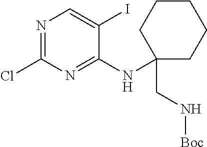 [SMILES: CC(C)(C)OC(=O)NCC1(Nc2nc(Cl)ncc2I)CCCCC1]

Step 1. To a solution of tert-butyl ((1-aminocyclohexyl)methyl)carbamate (5.24 g, 1 eq., 22.9 mmol) and K₂CO₃ (4.76 g, 1.5 eq., 34.4 mmol) in DMF (20 mL) was added 2,4-dichloro-5-iodopyrimidine (6.94 g, 1.1 eq., 25.2 mmol), and the resulting solution was stirred at 60° C. overnight. The mixture was diluted with EtOAc (100 mL) and washed with brine (3×120 mL). The organic phase was dried over anhydrous Na₂SO₄ and concentrated under reduced pressure. The residue was purified by silica-gel chromatography, eluting with PE/EtOAc (10:1). The desired fractions were combined, and then concentrated in vacuo to afford tert-butyl ((1-((2-chloro-5-iodopyrimidin-4-yl)amino)cyclohexyl)methyl)carbamate (2.8 g, 5.9 mmol, 26%, 98% purity) as a white solid. m/z (ES⁺) [M+H]⁺=466.95; HPLC tR=1.219 min.

### tert-butyl((1-((2-chloro-5-(3,3-diethoxyprop-1-yn-1-yl)pyrimidin-4-yl)amino)cyclohexyl)methyl)carbamate

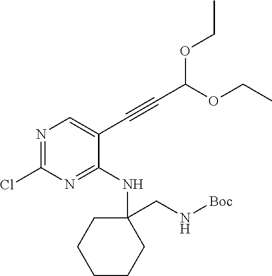 [SMILES: CCOC(C#Cc1cnc(Cl)nc1NC1(CNC(=O)OC(C)(C)C)CCCCC1)OCC]

Step 2. To a solution of tert-butyl ((1-((2-chloro-5-iodopyrimidin-4-yl)amino)cyclohexyl)methyl)carbamate (200 mg, 1 eq., 428 mol), 3,3-diethoxyprop-1-yne (93.4 mg, 1.7 eq., 728 mol) and N-ethyl-N-isopropylpropan-2-amine (95.8 mg, 1.73 eq., 741 mol) in THE (10 mL) was added copper(I) iodide (8.16 mg, 0.1 eq., 42.8 μmol) and Pd(Pph₃)₂Cl₂ (12.0 mg, 0.04 eq., 17.1 mol), and the resulting solution was stirred at r.t. for 6 h under N₂. The mixture was diluted with EtOAc (50 mL), then washed with brine (3×60 mL). The organic phase was dried over anhydrous Na₂SO₄ and concentrated under reduced pressure. The residue was purified by silica-gel chromatography, eluting with PE/EtOAc (10:1). The desired fractions were combined, and then concentrated in vacuo to afford tert-butyl ((1-((2-chloro-5-(3,3-diethoxyprop-1-yn-1-yl)pyrimidin-4-yl)amino)cyclohexyl)methyl)carbamate (180 mg, 368 mol, 86.0%, 95.6% purity) as a light yellow solid. m/z (ES⁺) [M+H]⁺=467.15; HPLC tR=1.204 min.

### tert-butyl((1-(2-chloro-6-(diethoxymethyl)-7H-pyrrolo[2,3-d]pyrimidin-7-yl)cyclohexyl)methyl)carbamate

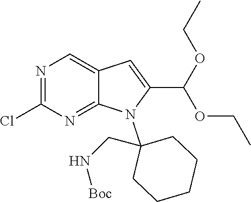 [SMILES: CCOC(OCC)c1cc2cnc(Cl)nc2n1C1(CNC(=O)OC(C)(C)C)CCCCC1]

Step 3. To a solution of tert-butyl ((1-((2-chloro-5-(3,3-diethoxyprop-1-yn-1-yl)pyrimidin-4-yl)amino)cyclohexyl)methyl)carbamate (300 mg, 1 eq., 642 μmol) in THF (20 mL) was added tetrabutylammonium fluoride (504 mg, 3 eq., 1.93 mmol), and the resulting solution was stirred at 60° C. for 4 hours. The mixture was diluted with EtOAc (100 mL) and washed with brine (3×120 mL). The organic phase was dried over anhydrous Na₂SO₄, then concentrated under reduced pressure. The crude product was purified by Prep-HPLC with the condition (column: Xselect CSH C18 OBD Column 30×150 mm 5 μm, n; mobile phase A: water (0.1% FA); mobile phase B: ACN; flow rate: 60 mL/min; gradient: 68% B to 78% B in 7 min; wavelength: 254; 220 nm; tR1 (min): 6.5). The desired fractions were combined, and then lyophilized to afford tert-butyl ((1-(2-chloro-6-(diethoxymethyl)-7H-pyrrolo[2,3-d]pyrimidin-7-yl)cyclohexyl)methyl)carbamate (140 mg, 0.28 mmol, 44%, 95% purity) as a light yellow solid. m/z (ES⁻) [M+H]⁺=467.15; HPLC tR=1.212 min.

### tert-butyl((1-(2-chloro-6-formyl-7H-pyrrolo[2,3-d]pyrimidin-7-yl)cyclohexyl)methyl)carbamate

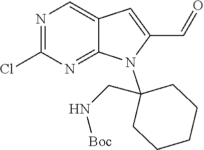 [SMILES: CC(C)(C)OC(=O)NCC1(n2c(C=O)cc3cnc(Cl)nc32)CCCCC1]

Step 4. To a solution of tert-butyl ((1-(2-chloro-6-(diethoxymethyl)-7H-pyrrolo[2,3-d]pyrimidin-7-yl)cyclohexyl)methyl)carbamate (70 mg, 1 eq., 0.15 mmol) in THF (2 mL) and H₂O (2 mL) was added acetic acid (27 mg, 3 eq., 0.45 mmol), and the resulting solution was stirred at 60° C. for 2 h. The mixture was concentrated under reduced pressure. The crude was diluted with EtOAc (20 mL) and washed with brine (3×30 mL). The organic phase was dried over anhydrous Na₂SO₄, then concentrated under reduced pressure to afford tert-butyl ((1-(2-chloro-6-formyl-7H-pyrrolo[2,3-d]pyrimidin-7-yl)cyclohexyl)methyl)carbamate (60 mg, 0.14 mmol, 91%, 89.1% purity) as a yellow solid. m/z (ES⁺) [M+H]⁺=393.10; HPLC tR=1.204 min.

### tert-butyl 2′-chloro-6′-oxo-6′H-spiro[cyclohexane-1,9′-pyrazino[1′,2′:1,5]pyrrolo[2,3-d]pyrimidine]-7′(8′H)-carboxylate

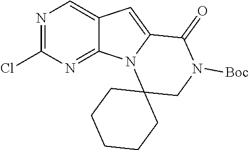 [SMILES: CC(C)(C)OC(=O)N1CC2(CCCCC2)n2c(cc3cnc(Cl)nc32)C1=O]

Step 5. To a mixture of tert-butyl ((1-(2-chloro-6-formyl-7H-pyrrolo[2,3-d]pyrimidin-7-yl)cyclohexyl)methyl)carbamate (38 mg, 1.5 eq., 98 mol) in DMF (6 mL) was added oxone (40 mg, 1 eq., 65 mol) at 25° C., and the resulting solution was stirred for 3 hours. The desired product was detected by LC/MS. The mixture was diluted with EtOAc (20 mL) and washed with brine (3×20 mL). The organic phase was dried over anhydrous Na₂SO₄ and concentrated under reduced pressure. The crude product tert-butyl 2′-chloro-6′-oxo-6′H-spiro[cyclohexane-1,9′-pyrazino[1,2′:1,5]pyrrolo[2,3-d]pyrimidine]-7′(8′H)-carboxylate (30 mg, 61 μmol, 94%, 80% purity) was used in the next step directly without further purification. m/z (ES⁺) [M+H]⁺=391.10; HPLC tR=1.159 min.

### 2′-(((3S,4R)-3-hydroxytetrahydro-2H-pyran-4-yl)amino)-7′,8′-dihydro-6′H-spiro[cyclohexane-1,9′-pyrazino[1′,2′:1,5]pyrrolo[2,3-d]pyrimidin]-6′-one (Compound I-22)

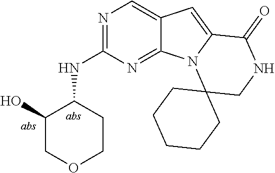 [SMILES: O=C1NCC2(CCCCC2)n2c1cc1cnc(N[C@@H]3CCOC[C@H]3O)nc12]

Step 6. To a solution of tert-butyl 2′-chloro-6′-oxo-6′H-spiro[cyclohexane-1,9′-pyrazino[1′,2′:1,5]pyrrolo[2,3-d]pyrimidine]-7′(8′H)-carboxylate (40 mg, 1 eq., 0.10 mmol) and N-ethyl-N-isopropylpropan-2-amine (0.13 g, 10 eq., 1.0 mmol) in NMP (3 mL) was added (3S,4R)-3-hydroxytetrahydro-2H-pyran-4-aminium chloride (63 mg, 4 eq., 0.41 mmol), and the resulting solution was stirred overnight at 130° C. The solution was diluted with EtOAc (20 mL) and washed with brine (3×30 mL). The resulting mixture was concentrated under reduced pressure. The crude product was purified by reverse-phase flash chromatography (column: C18 silica gel; mobile phase: ACN in water; gradient: 0% to 100% in 30 min; UV detector wavelength: 254 nm). The desired fractions were combined, and then concentrated in vacuo to afford 2′-(((3S,4R)-3-hydroxytetrahydro-2H-pyran-4-yl)amino)-7′,8′-dihydro-6′H-spiro[cyclohexane-1,9′-pyrazino[1′,2′:1,5]pyrrolo[2,3-d]pyrimidin]-6′-one (5.4 mg, 14 mol, 14%, 98.0% purity) as a white solid. m/z (ES⁺) [M+H]⁺=372.10; HPLC tR=0.537 min.

### Example 5

### (1R,2R)-2-((5-chloro-4-(4-fluoro-1-isopropyl-2-methyl-1H-benzo[d]imidazol-6-yl)pyrimidin-2-yl)amino)cyclohexan-1-ol (Compound I-32)

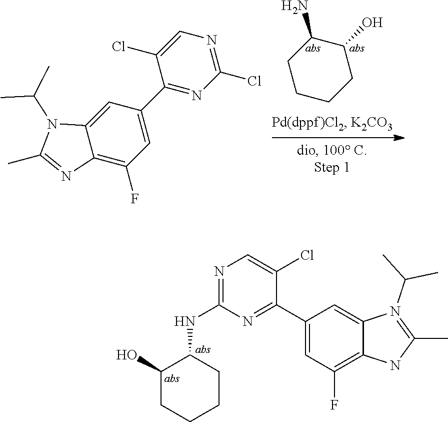

Step 1. (1R,2R)-2-aminocyclohexan-1-ol (122 mg, 1 eq., 1.06 mmol), K₂CO₃ (439 mg, 3 eq., 3.18 mmol) and Pd(dppf)Cl₂ (86.6 mg, 0.1 eq., 106 μmol) were added to a solution of 6-(2,5-dichloropyrimidin-4-yl)-4-fluoro-1-isopropyl-2-methyl-1H-benzo[d]imidazole (360 mg, 1 eq., 1.06 mmol) in 1,4-dioxane (10 mL) under nitrogen atmosphere. The reaction mixture was heated at 100° C. for 16 hours with vigorous stirring. The mixture was then concentrated in vacuo. The resulting crude material was purified by preparative HPLC (column: XBridge Prep OBD C18 Column, 30×150 mm, 5 μm; mobile phase A: water (10 mM NH₄HCO₃+0.1% NH₃·H₂O); mobile phase B: ACN; flow rate: 60 mL/min; gradient: 27% B to 57% B in 8 min, 57% B; wavelength: 220 nm; tR1 (min): 7.58). The desired fractions were combined, and then lyophilized to afford (1R,2R)-2-((5-chloro-4-(4-fluoro-1-isopropyl-2-methyl-1H-benzo[d]imidazol-6-yl)pyrimidin-2-yl)amino)cyclohexan-1-ol (77.6 mg, 0.18 mmol, 17%, 99% purity) as an off-white solid. m/z (ES⁺) [M+H]⁺=418.20; HPLC tR=0.701 min. ¹H NMR (400 MHz, DMSO-d₆) 8.38 (s, 1H), 7.92 (s, 1H), 7.32 (d, J=51.8 Hz, 2H), 4.80 (h, J=6.9 Hz, 1H), 4.59 (d, J=4.9 Hz, 1H), 3.60 (s, 1H), 3.41 (s, 1H), 2.62 (s, 3H), 2.17-1.80 (m, 2H), 1.58 (d, J=6.8 Hz, 7H), 1.20 (t, J=8.9 Hz, 4H).

### Example 6

### (4R,5R)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol and (4S,5S)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol (Compounds I-36 and I-37)

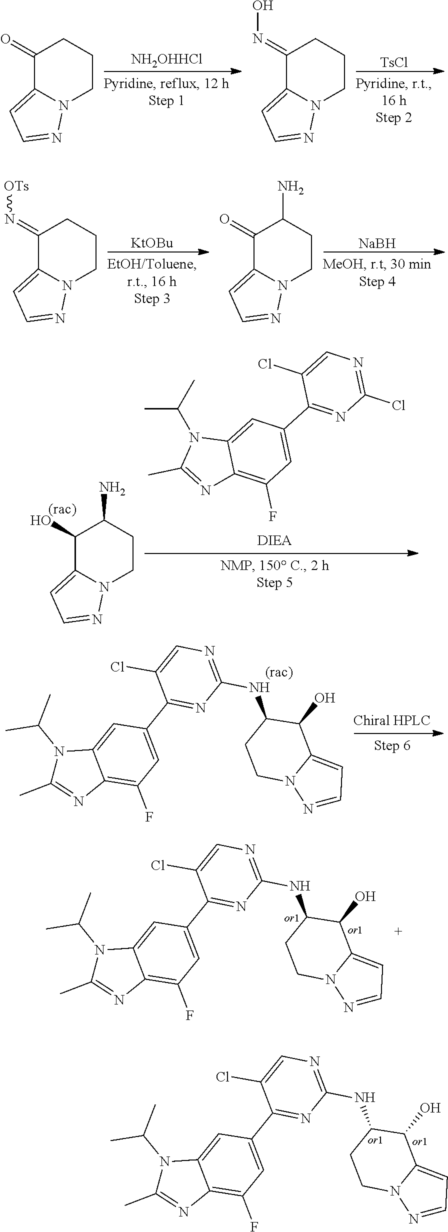

### (E)-6,7-dihydropyrazolo[1,5-a]pyridin-4 (5H)-one oxime

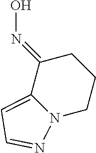 [SMILES: O/N=C1\CCCn2nccc21]

Step 1. To a solution of 6,7-dihydropyrazolo[1,5-a]pyridin-4 (5H)-one (5.0 g, 37 mmol) in pyridine (50 mL) was added hydroxylamine hydrochloride (2.6 g, 37 mmol), and the solution was stirred at 80° C. for 16 hours. The resulting mixture was concentrated under reduced pressure. The crude product was diluted with water (100 mL) and extracted with DCM (3×100 mL). The combined organic layer was dried over anhydrous Na₂SO₄ and then concentrated under reduced pressure to give (E)-6,7-dihydropyrazolo[1,5-a]pyridin-4 (5H)-one oxime (6.5 g, 37 mmol, 85%). m/z (ES⁺) [M+H]⁻=152.05; HPLC tR=0.404 min

### (E)-6,7-dihydropyrazolo[1,5-a]pyridin-4 (5H)-one O-tosyl oxime

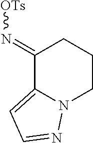 [SMILES: Cc1ccc(S(=O)(=O)ON=C2CCCn3nccc32)cc1]

Step 2. To a stirring solution of (E)-6,7-dihydropyrazolo[1,5-a]pyridin-4 (5H)-one oxime (5.5 g, 36 mmol) in anhydrous pyridine (100 mL) was added TsCl (6.9 g, 36 mmol), and the resulting solution was stirred at 25° C. for 48 hours. The reaction progress was monitored by LC/MS. Upon completion of reaction, the resulting mixture was concentrated under reduced pressure to give a crude product, which was further purified by flash chromatography (eluent: ACN in water; gradient: 0% to 100% in 20 min). The desired fractions were combined, and then concentrated in vacuo to afford the desired compound, (E)-6,7-dihydropyrazolo[1,5-a]pyridin-4 (5H)-one O-tosyl oxime (9 g, 0.03 mol, 80%, 97.2% purity). m/z (ES⁺) [M+H]⁺=306.05; HPLC tR=0.884 min

### 5-amino-6,7-dihydropyrazolo[1,5-a]pyridin-4 (5H)-one

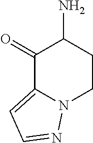 [SMILES: NC1CCn2nccc2C1=O]

Step 3. To a stirring solution of (E)-6,7-dihydropyrazolo[1,5-a]pyridin-4 (5H)-one O-tosyl oxime (5.0 g, 16 mmol) in EtOH (50 mL) and toluene (100 mL) was added potassium tert-butoxide (3.7 g, 33 mmol), and the resulting mixture was stirred at 25° C. for 2 hours. The reaction progress was monitored by LC/MS. Upon completion of reaction, the resulting mixture was filtered, and the residue was washed with hexane (3×5 mL). To the filtrate was added 36% HCl (2.5 mL) dropwise, and a white precipitate then formed. After the mixture had been stirred at r.t. for 16 hours, the solid was filtered off and then washed with acetone (2×5 mL) to afford the desired product, 5-amino-6,7-dihydropyrazolo[1,5-a]pyridin-4 (5H)-one (0.82 g, 5.4 mmol, 33%, 99.9% purity). m/z (ES⁺) [M+H]⁺=151.95; HPLC tR=0.475 min.

### rac-(4S,5S)-5-amino-4,5,6,7-tetrahydropyrazolo[1,5-a]pyridin-4-ol

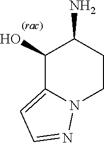 [SMILES: N[C@H]1CCn2nccc2[C@H]1O]

Step 4. To a solution of 5-amino-6,7-dihydropyrazolo[1,5-a]pyridin-4 (5H)-one (600 mg, 3.97 mmol) in MeOH (10 mL) was added NaBH₄ (225 mg, 5.95 mmol), and the mixture was stirred at r.t. for 30 min. The residue was purified by reverse-phase flash chromatography (column: C18 silica gel; mobile phase: ACN in water; gradient: 0% to 100% in 20 min; wavelength: 254 nm). The resulting liquid was concentrated under reduced pressure to afford rac-(4S,5S)-5-amino-4,5,6,7-tetrahydropyrazolo[1,5-a]pyridin-4-ol (260 mg, 1.56 mmol, 39.3%, 91.9% purity). m/z (ES⁺) [M+H]⁺=154.00; HPLC tR=0.262 min.

### rac-(4R,5R)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol

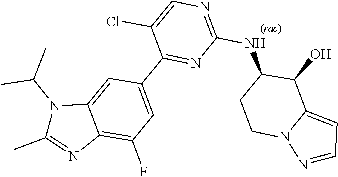 [SMILES: Cc1nc2c(F)cc(-c3nc(N[C@@H]4CCn5nccc5[C@@H]4O)ncc3Cl)cc2n1C(C)C]

Step 5. To a solution of rac-(4R,5R)-5-amino-4,5,6,7-tetrahydropyrazolo[1,5-a]pyridin-4-ol (100 mg, 653 mol) and 6-(2,5-dichloropyrimidin-4-yl)-4-fluoro-1-isopropyl-2-methyl-1H-benzo[d]imidazole (221 mg, 653 mol) in NMP (3 mL) was added N-ethyl-N-isopropylpropan-2-amine (844 mg, 6.53 mmol), and the resulting solution was stirred at 150° C. for 2 hours. The residue was purified by reverse-phase flash chromatography (column: C18 silica gel; mobile phase: water in ACN; gradient: 0% to 100% in 30 min; wavelength: 254 nm). The desired fractions were combined, and then concentrated in vacuo to afford rac-(4R,5R)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol (120 mg, 262 μmol, 40.1%, 99.4% purity) as a yellow solid. m/z (ES⁺) [M+H]⁻=456.10; HPLC tR=0.822 min.

### rel-(4R,5R)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol & rel-(4S,5S)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol

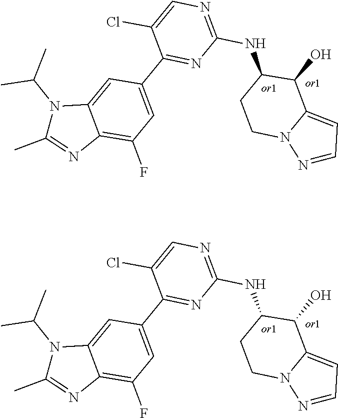 [SMILES: Cc1nc2c(F)cc(-c3nc(N[C@@H]4CCn5nccc5[C@@H]4O)ncc3Cl)cc2n1C(C)C ; Cc1nc2c(F)cc(-c3nc(N[C@H]4CCn5nccc5[C@H]4O)ncc3Cl)cc2n1C(C)C]

Step 6. The mixture rac-(4R,5R)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol (120 mg) was purified by chiral preparative HPLC (column: CHIRALPAK IG, 2×25 cm, 5 μm; mobile phase A: Hex (0.2% TEA) HPLC; mobile phase B: EtOH/DCM (1:1) HPLC; flow rate: 20 mL/min; gradient: 60% B to 60% B in 12 min; wavelength: 220 nm & 254 nm; tR1 (min): 4.59; tR2 (min): 7.73; sample solvent: EtOH/DCM (1:1) HPLC; injection volume: 1 mL; number of runs: 4). The desired fractions were combined, and then concentrated in vacuo to afford (tR1) rel-(4R,5R)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol (33.3 mg, 69.8 μmol, 26.5%, 95.5% purity) as a yellow solid. m/z (ES⁺) [M+H]⁺=456.15; HPLC tR=1.571 min ¹H NMR (400 MHz, DMSO-d6) 8.48 (s, 1H), 7.95 (s, 1H), 7.66 (s, 1H), 7.40 (d, J=1.8 Hz, 2H), 6.22 (d, J=1.8 Hz, 1H), 5.67 (d, J=6.5 Hz, 1H), 4.81 (dt, J=14.3, 7.0 Hz, 2H), 4.28 (dd, J=12.1, 6.2 Hz, 1H), 4.14 (d, J=8.2 Hz, 2H), 2.62 (s, 3H), 2.38 (d, J=12.0 Hz, 1H), 2.08 (s, 1H), 1.58 (d, J=6.8 Hz, 6H). Next, the same purification (tR2) afforded rel-(4S,5S)-5-((5-chloro-4-(4-fluoro-1-isopropyl-2-methyl-1H-benzo[d]imidazol-6-yl)pyrimidin-2-yl)amino)-4,5,6,7-tetrahydropyrazolo[1,5-a]pyridin-4-ol (37.5 mg, 81.8 mol, 62%, 99.4% purity) as a white solid. m/z (ES⁺) [M+H]⁺=456.10; HPLC tR=1.544 min ¹H NMR (400 MHz, DMSO-d₆) 8.48 (s, 1H), 7.97 (s, 1H), 7.67 (s, 1H), 7.40 (d, J=1.8 Hz, 2H), 6.22 (s, 1H), 5.69 (s, 1H), 4.87-4.75 (m, 2H), 4.29 (d, J=11.6 Hz, 1H), 4.12 (s, 2H), 2.62 (s, 3H), 2.35 (d, J=12.0 Hz, 1H), 2.07 (s, 1H), 1.58 (d, J=6.9 Hz, 6H).

### Example 7

### (1S,3R,4R)-4-((5-chloro-4-(4-fluoro-1-isopropyl-2-methyl-1H-benzo[d]imidazol-6-yl)pyrimidin-2-yl)amino)cyclohexane-1,3-diol (Compound I-95)

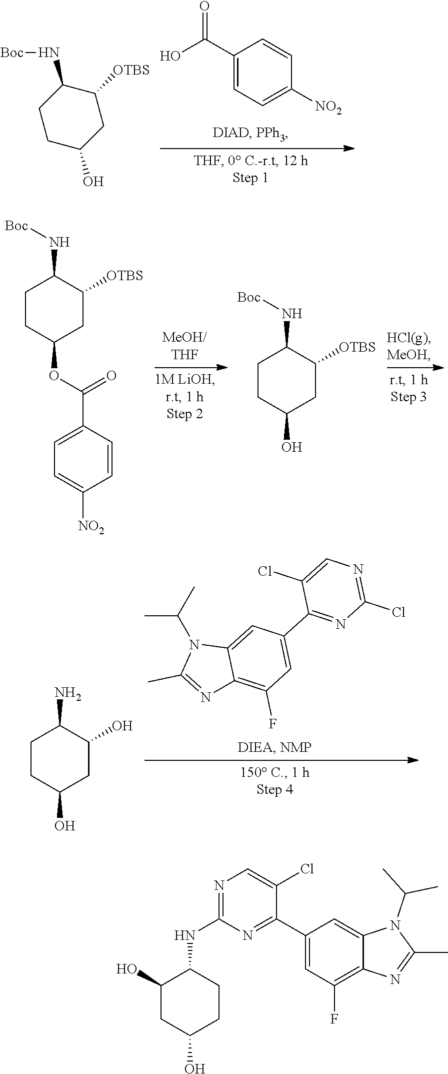

### (1S,3R,4R)-4-((tert-butoxycarbonyl)amino)-3-((tertbutyldimethylsilyl)oxy)cyclohexyl 4-nitrobenzoate

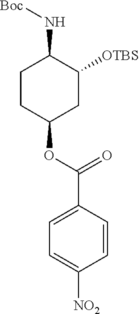 [SMILES: CC(C)(C)OC(=O)N[C@@H]1CC[C@H](OC(=O)c2ccc([N+](=O)[O-])cc2)C[C@H]1O[Si](C)(C)C(C)(C)C]

Step 1. To a mixture of tert-butyl ((1R,2R,4R)-2-((tert-butyldimethylsilyl)oxy)-4-hydroxycyclohexyl)carbamate (300 mg, 0.87 mmol), 4-nitrobenzoic acid (174 mg, 1.04 mmol) and Ph₃P (524 mg, 2.00 mmol) in THF (10 mL) was added DIAD (404 mg, 2.00 mmol) dropwise at 0° C. under nitrogen atmosphere. The mixture was slowly warmed to room temperature and stirred at 25° C. for 12 hours. The resulting mixture was diluted with H₂O (30 mL), and the aq. phase was extracted with EtOAc (3×30 mL). The combined organic layers were washed with brine, dried over sodium sulfate, filtered, and then concentrated in vacuo. The residue was purified by preparative TLC, eluting with PE/EtOAc (4:1). The desired fractions were combined, and then concentrated in vacuo to afford (1S,3R,4R)-4-((tert-butoxycarbonyl)amino)-3-((tertbutyldimethylsilyl)oxy)cyclohexyl 4-nitrobenzoate (220 mg, 51.2%) as an oil. m/z (ES⁺) [M+H]⁺=495.35; HPLC tR=1.327 min.

### tert-butyl ((1R,2R,4S)-2-((tert-butyldimethylsilyl)oxy)-4-hydroxycyclohexyl)carbamate

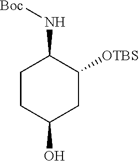 [SMILES: CC(C)(C)OC(=O)N[C@@H]1CC[C@H](O)C[C@H]1O[Si](C)(C)C(C)(C)C]

Step 2. A resealable reaction vial was charged with (1S,3R,4R)-4-((tert-butoxycarbonyl)amino)-3-((tert-butyldimethylsilyl)oxy)cyclohexyl 4-nitrobenzoate (220 mg, 0.45 mmol), LiOH (1.33 mL, 1.33 mmol), THF/MeOH (2 mL) and a stir bar, and then evacuated and purged with nitrogen three times, and the mixture was stirred for 1 hour at 25° C. The resulting mixture was diluted with H₂O (5 mL), then adjusted to pH 9~-10 with 1M LiOH, and the aq. phase was extracted with EtOAc (3×30 mL). The combined organic layers were washed with brine, dried over sodium sulfate, filtered, and then concentrated in vacuo. This afforded crude tert-butyl ((1R,2R,4S)-2-((tert-butyldimethylsilyl)oxy)-4-hydroxycyclohexyl)carbamate (158 mg, 99%) as an oil. m/z (ES⁺) [M+H]⁺=346.25; HPLC tR=1.160 min.

### (1S,3R,4R)-4-aminocyclohexane-1,3-diol

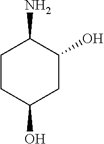

Step 3. A resealable reaction vial was charged with tert-butyl ((1R,2R,4S)-2-((tert-butyldimethylsilyl)oxy)-4-hydroxycyclohexyl)carbamate (70 mg, 0.20 mmol), MeOH (2 mL), 4 M HCl in dioxane (1 mL) and a stir bar, and then evacuated and purged with nitrogen three times, and the mixture was stirred for 1 hour at 25° C. The resulting mixture was concentrated in vacuo to afford (1S,3R,4R)-4-aminocyclohexane-1,3-diol (20 mg, 75%) as a white solid. m/z (ES⁺) [M+H]⁺=132.15; HPLC tR=0.168 min.

### (1S,3R,4R)-4-((5-chloro-4-(4-fluoro-1-isopropyl-2-methyl-1H-benzo[d]imidazol-6-yl)pyrimidin-2-yl)amino)cyclohexane-1,3-diol

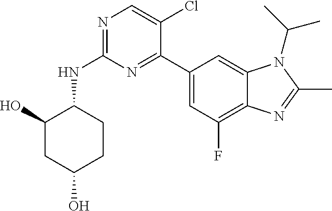 [SMILES: Cc1nc2c(F)cc(-c3nc(N[C@@H]4CC[C@H](O)C[C@H]4O)ncc3Cl)cc2n1C(C)C]

Step 4. A resealable reaction vial was charged with (1S,3R,4R)-4-aminocyclohexane-1,3-diol (20 mg, 0.15 mmol), 6-(2,5-dichloropyrimidin-4-yl)-4-fluoro-1-isopropyl-2-methyl-1H-benzo[d]imidazole (62 mg, 0.18 mmol), DIEA (120 mg, 0.91 mmol), NMP (1 mL) and a stir bar, and then evacuated and purged with nitrogen three times, and the mixture was stirred at 150° C. for 1 hour. The residue was purified by reverse-phase flash chromatography (column: C18 silica gel; mobile phase: ACN in water; gradient: 10% to 50% in 20 min; wavelength: 254 nm), and then concentrated in vacuo. The resulting crude material was purified by preparative HPLC (column: XBridge Prep OBD C18 Column, 30×150 mm, 5 μm; mobile phase A: water (10 mM NH₄HCO₃+0.1% NH₃·H₂O), mobile phase B: ACN; flow rate: 60 mL/min; gradient: 15% B to 40% B in 8 min, 40% B; wavelength: 220 nm; tR1 (min): 8.00; injection volume: 0.8 mL; number of runs: 2). The desired fractions were combined, and then lyophilized to afford (1S,3R,4R)-4-((5-chloro-4-(4-fluoro-1-isopropyl-2-methyl-1H-benzo[d]imidazol-6-yl)pyrimidin-2-yl)amino)cyclohexane-1,3-diol (28.4 mg, 43%) as a white amorphous solid. m/z (ES⁺) [M+H]⁺=434.10; HPLC tR=0.852 min. ¹H NMR (400 MHz, DMSO-d) δ 8.39 (s, 1H), 7.92 (s, 1H), 7.30 (s, 2H), 4.80 (p, J=6.8 Hz, 1H), 4.51 (d, J=5.1 Hz, 1H), 4.42 (d, J=2.9 Hz, 1H), 3.92 (s, 1H), 3.79 (s, 1H), 3.68-3.56 (m, 1H), 2.62 (s, 3H), 1.99-1.72 (m, 2H), 1.58 (d, J=6.9 Hz, 8H), 1.50-1.31 (m, 2H).

### Example 8

### (1S,3S,4S)-4-((5-chloro-4-(4-fluoro-1-isopropyl-2-methyl-1H-benzo[d]imidazol-6-yl) pyrimidin-2-yl)amino)-3-hydroxycyclohexylmethyl carbamate (Compound I-113)

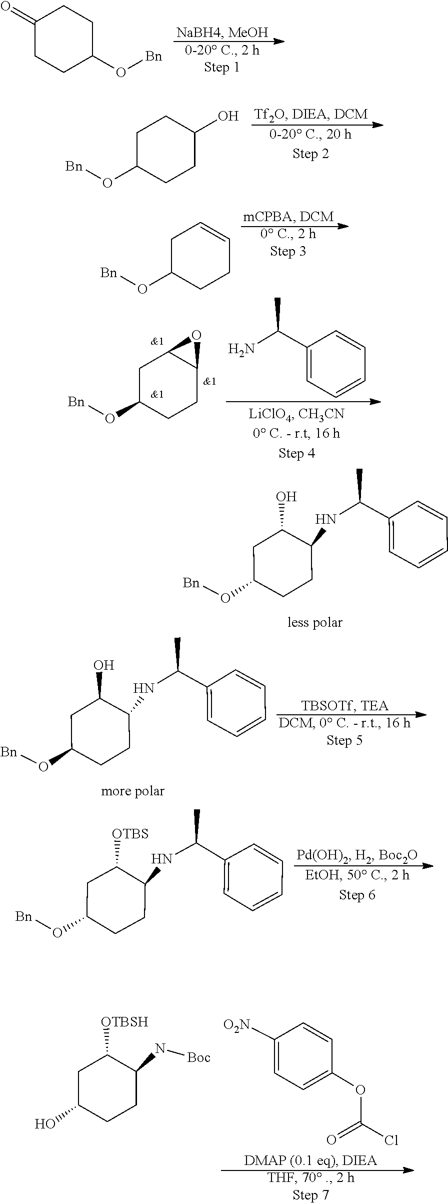

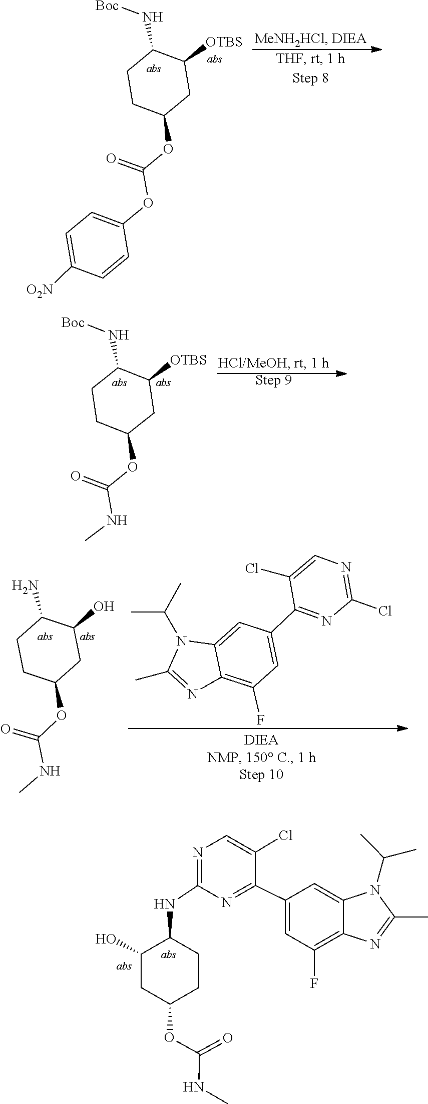

### 4-(benzyloxy) cyclohexan-1-ol

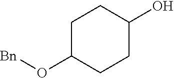 [SMILES: OC1CCC(OCc2ccccc2)CC1]

Step 1. To an ice-bath cooled solution of 4-(benzyloxy) cyclohexan-1-one (50 g, 0.24 mol) in MeOH (500 mL), was added NaBH₄ (14 g, 0.37 mol) in several portions during a period of 10 min, and the resulting solution was stirred at 20° C. for 2 hours. The reaction mixture was diluted with sat'd. aq. NH₄Cl (100 mL), and the aq. phase was extracted with EtOAc (3×600 mL). The combined organic layers were washed with brine, dried over sodium sulfate, filtered, and then concentrated in vacuo. The residue was purified by silica-gel chromatography, eluting with PE/EtOAc (30:1). The desired fractions were combined, and then concentrated in vacuo to afford 4-(benzyloxy) cyclohexan-1-ol (49 g, 97%) as a colorless oil. m/z (ES⁺) [M+H]⁺=207.10; HPLC tR=0.767 min.

### ((cyclohex-3-en-1-yloxy) methyl) benzene

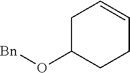 [SMILES: C1=CCC(OCc2ccccc2)CC1]

Step 2. A round-bottomed flask was charged with 4-(benzyloxy) cyclohexan-1-ol (49 g, 0.24 mol) in DCM (500 mL), DIEA (46 g, 0.36 mol) and a stir bar, and then evacuated and purged with nitrogen three times. Tf₂O (74 g, 0.26 mol) was added dropwise at 0° C., and the reaction was stirred at 0° C. for 30 min. The resulting mixture was stirred at 20° C. for 16 hours under nitrogen atmosphere, and then diluted with water (150 mL) at 0° C. The aq. phase was extracted with DCM (3×150 mL). The combined organic layers were washed with brine, dried over sodium sulfate, filtered, and then concentrated in vacuo. The crude product was purified by silica-gel chromatography (500 g column), eluting with PE/EtOAc (16:1). The desired fractions were combined, and then concentrated in vacuo to afford ((cyclohex-3-en-1-yloxy) methyl) benzene (37 g, 83%) as a yellow oil. m/z (ES⁺) [M+H]⁺=189.15; HPLC tR=1.285 min.

### rac-(1R,3R,6S)-3-(benzyloxy)-7-oxabicyclo[4.1.0]heptane

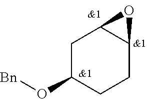 [SMILES: c1ccc(CO[C@@H]2CC[C@@H]3O[C@@H]3C2)cc1]

Step 3. To a solution of ((cyclohex-3-en-1-yloxy) methyl) benzene (39 g, 0.21 mol) in DCM (600 mL) was added m-CPBA (71 g, 0.41 mol) at 0° C., and the resulting mixture was stirred for 2 hours. The mixture was then diluted with H₂O (50 mL), and the aq. phase was extracted with EtOAc (3×200 mL). The combined organic layers were washed with brine, dried over sodium sulfate, filtered, and then concentrated in vacuo. The residue was purified by silica-gel chromatography (column: 60 g), eluting with PE/EtOAc (30:1) to afford a mixture of products (30 g). This mixture was purified by flash chromatography, eluting with PE/EtOAc (10:1). The desired fractions were combined, and then concentrated in vacuo to afford rac-(1R,3R,6S)-3-(benzyloxy)-7-oxabicyclo[4.1.0]heptane (4 g, 20%) as a colorless oil. m/z (ES⁺) [M+H]⁺=205.15; HPLC tR=1.002 min.

### (1R,2R,5R)-5-(benzyloxy)-2-(((S)-1-phenyl ethyl) amino) cyclohexan-1-ol and (1S,2S,5S)-5-(benzyloxy)-2-(((S)-1-phenylethyl) amino) cyclohexan-1-ol mixture

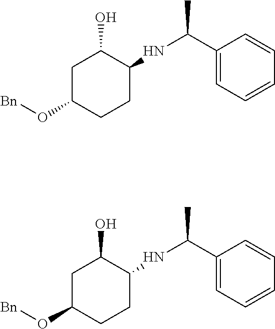 [SMILES: C[C@H](N[C@H]1CC[C@H](OCc2ccccc2)C[C@@H]1O)c1ccccc1 ; C[C@H](N[C@@H]1CC[C@@H](OCc2ccccc2)C[C@H]1O)c1ccccc1]

Step 4. To a mixture of rac-(1R,3R,6S)-3-(benzyloxy)-7-oxabicyclo [4.1.0]heptane (4.4 g, 22 mmol) and (S)-1-phenylethan-1-amine (3.5 g, 29 mmol) in ACN (35 mL) was added lithium perchlorate (4.6 g, 43 mmol) in portions at 0° C. under nitrogen atmosphere. The mixture was stirred at r.t. for 16 hours. Then, the mixture was diluted with H₂O (50 mL), and the aq. phase was extracted with EtOAc (3×100 mL). The combined organic layers were washed with brine, dried over sodium sulfate, filtered, and then concentrated in vacuo. This afforded the desired product as a mixture (total: 6.3 g) of (1R,2R,5R)-5-(benzyloxy)-2-(((S)-1-phenyl ethyl) amino) cyclohexan-1-ol and (1S,2S,5S)-5-(benzyloxy)-2-(((S)-1-phenylethyl) amino) cyclohexan-1-ol, which used directly in the next step without further purification. m/z (ES⁺) [M+H]⁺=326.15, HPLC tR=0.917 min.

### (1S,2S,4S)-4-(benzyloxy)-2-((tert-butyldimethylsilyl)oxy)-N—((S)-1-phenylethyl)cyclohexan-1-amine

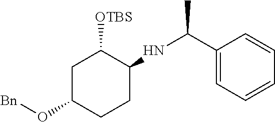 [SMILES: C[C@H](N[C@H]1CC[C@H](OCc2ccccc2)C[C@@H]1O[Si](C)(C)C(C)(C)C)c1ccccc1]

Step 5. To an ice-cold solution of a mixture (6.3 g, 19 mmol) of (1R,2R,5R)-5-(benzyloxy)-2-(((S)-1-phenyl ethyl) amino) cyclohexan-1-ol and (1S,2S,5S)-5-(benzyloxy)-2-(((S)-1-phenylethyl) amino) cyclohexan-1-ol and triethylamine (5.9 g, 58 mmol) in DCM (70 mL), was added TBSOTf (15 g, 58 mmol). The resulting mixture was stirred at r.t. for 16 hours. Next, the reaction mixture was diluted with H₂O (50 mL), and the aq. phase was extracted with EtOAc (3×100 mL). The combined organic layers were washed with brine, dried over sodium sulfate, filtered, and then concentrated in vacuo. The crude product was purified by silica-gel chromatography (column: 30 g), eluting with PE/EtOAc (gradient: 7:1 to 3:1). The desired fractions were combined, and then concentrated in vacuo to afford (1 S,2S,4S)-4-(benzyloxy)-2-((tert-butyldimethylsilyl)oxy)-N—((S)-1-phenylethyl)cyclohexan-1-amine (4.4 g, 94%) as a dark yellow oil. m/z (ES⁺) [M+H]⁻=440.35; HPLC tR=1.118 min. tert-butyl ((1S,2S,4S)-2-((tert-butyldimethylsilyl)oxy)-4-hydroxycyclohexyl)carbamate

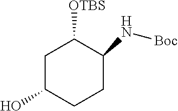 [SMILES: CC(C)(C)OC(=O)N[C@H]1CC[C@H](O)C[C@@H]1O[Si](C)(C)C(C)(C)C]

Step 6. A stirred mixture of (1S,2S,4S)-4-(benzyloxy)-2-((tert-butyldimethylsilyl) oxy)-N—((S)-1-phenylethyl) cyclo-hexan-1-amine (4.2 g, 9.6 mmol), (Boc)₂O (2.2 g, 10 mmol) and Pd(OH)₂ (4.2 g, 30 mmol) in EtOH (50 mL) was treated with H₂ at 50° C. for 2 hours. The resulting mixture was filtered through a pad of Celite, which was washed with EtOAc, and the filtrate was concentrated in vacuo. This afforded the crude product, tert-butyl ((1S,2S,4S)-2-((tert-butyldimethylsilyl)oxy)-4-hydroxycyclohexyl)carbamate (2.9 g, 88%), as a grey solid. m/z (ES⁺) [M+H]⁺=346.30; HPLC tR=1.135 min.

### tert-butyl ((1S,2S,4S)-2-((tert-butyldimethylsilyl) oxy)-4-(((4-nitrophenoxy) carbonyl) oxy) cyclohexyl) carbamate

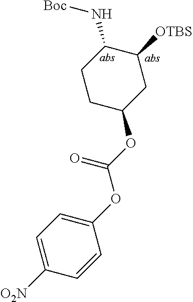 [SMILES: CC(C)(C)OC(=O)N[C@H]1CC[C@H](OC(=O)Oc2ccc([N+](=O)[O-])cc2)C[C@@H]1O[Si](C)(C)C(C)(C)C]

Step 7. To a mixture of tert-butyl ((1S,2S,4S)-2-((tert-butyldimethylsilyl) oxy)-4-hydroxycyclohexyl) carbamate (500 mg, 1.45 mmol), DMAP (18 mg, 145 mol) and DIEA (561 mg, 4.34 mmol) in THE (10 mL) was added a solution of 4-nitrophenyl chloroformate (583 mg, 2.89 mmol) dissolved in 1 mL THF, dropwise at 0° C. under nitrogen atmosphere. The mixture was stirred at 70° C. for 2 hours. Then, the mixture was diluted with H₂O (20 mL), and the aq. phase was extracted with EtOAc (3×30 mL). The combined organic layers were washed with brine, dried over sodium sulfate, filtered, and then concentrated in vacuo. The residue was purified by reverse-phase flash chromatography (column: C18 silica gel; mobile phase: ACN in water; gradient: 90% to 100% in 6 min; wavelength: 254 nm). The desired fractions were combined, and then concentrated in vacuo to afford tert-butyl ((1S,2S,4S)-2-((tert-butyldimethylsilyl) oxy)-4-(((4-nitrophenoxy) carbonyl) oxy) cyclohexyl) carbamate (495 mg, 67.0%) as a tan oil. m/z (ES⁺) [M-t-Bu+H]⁻=455.15; HPLC tR=1.548 min.

### (1S,3S,4S)-4-((tert-butoxycarbonyl) amino)-3-((tert-butyl dimethylsilyl)oxy)cyclohexyl methylcarbamate

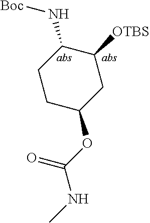 [SMILES: CNC(=O)O[C@H]1CC[C@H](NC(=O)OC(C)(C)C)[C@@H](O[Si](C)(C)C(C)(C)C)C1]

Step 8. To a solution of methylamine hydrochloride (128 mg, 1.90 mmol) in THE (7 mL), was added DIEA (368 mg, 2.85 mmol), and the resulting mixture was stirred at r.t. for 10 min. Next, tert-butyl((1S,2S,4S)-2-((tert-butyldimethylsilyl)oxy)-4-(((4-nitrophenoxy)carbonyl)oxy)cyclohexyl)carbamate (485 mg, 0.95 mmol) was added, and the mixture was stirred at r.t. for 1 hour. Then, the mixture was diluted with H₂O (15 mL) and treated with 5 mL 1M LiOH, and the aq. phase was extracted with EtOAc (3×20 mL). The combined organic layers were washed with brine, dried over sodium sulfate, filtered, and then concentrated in vacuo to afford the crude product, (1S,3S,4S)-4-((tert-butoxycarbonyl) amino)-3-((tert-butyldimethylsilyl)oxy)cyclohexyl methylcarbamate (330 mg, 86.3%), as a yellow solid. m/z (ES⁺) [M+Na]⁺=425.10; HPLC tR=1.383 min.

### (1S,3S,4S)-4-amino-3-hydroxycyclohexyl methylcarbamate

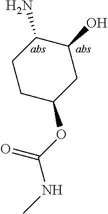

Step 9. To a solution of (1S,3S,4S)-4-((tert-butoxycarbonyl) amino)-3-((tert-butyldimethylsilyl) oxy)cyclohexyl methylcarbamate (160 mg, 397 μmol) in MeOH (2 mL) was added 4 M HCl in dioxane (2 mL), and the resulting mixture was stirred at r.t. for 1 hour. Concentration in vacuo afforded the crude product, (1S,3S,4S)-4-amino-3-hydroxycyclohexyl methylcarbamate (68 mg, 91%), as a yellow solid. m/z (ES⁺) [M+H]⁺=189.20; HPLC tR=0.143 min.

### (1S,3S,4S)-4-((5-chloro-4-(4-fluoro-1-isopropyl-2-methyl-1H-benzo[d]imidazol-6-yl) pyrimidin-2-yl)amino)-3-hydroxycyclohexylmethyl carbamate

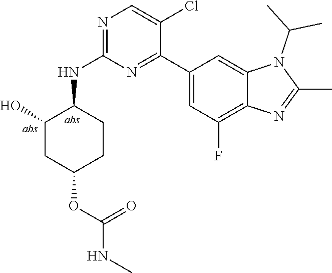 [SMILES: CNC(=O)O[C@H]1CC[C@H](Nc2ncc(Cl)c(-c3cc(F)c4nc(C)n(C(C)C)c4c3)n2)[C@@H](O)C1]

Step 10. To a solution of 6-(2,5-dichloropyrimidin-4-yl)-4-fluoro-1-isopropyl-2-methyl-1H-benzo[d]imidazole (0.12 g, 0.35 mmol) in NMP (4 mL) were added (1S,3S,4S)-4-amino-3-hydroxycyclohexyl methylcarbamate (63 mg, 0.33 mmol) and DIEA (0.17 g, 1.3 mmol), and the mixture was heated at 150° C. for 1 hour. The resulting crude material was purified by prep-HPLC (column: XBridge Prep OBD C18 Column, 30×150 mm, 5 μm; mobile phase A: water (10 mM NH₄HCO₃); mobile phase B: ACN; flow rate: 60 mL/min; gradient: 26% B to 48% B in 8 min, 48% B); lyophilization afforded the desired final product, (1S,3S,4S)-4-((5-chloro-4-(4-fluoro-1-isopropyl-2-methyl-1H-benzo[d]imidazol-6-yl) pyrimidin-2-yl)amino)-3-hydroxycyclohexylmethyl carbamate (33.4 mg, 20%), as an off-white solid. m/z (ES⁺) [M+H]⁺=491.15; HPLC tR=1.118 min. ¹H NMR (400 MHz, DMSO-d₆) 8.39 (s, 1H), 7.91 (s, 1H), 7.31 (d, J=39.7 Hz, 2H), 7.00-6.93 (m, 1H), 4.86-4.75 (m, 2H), 4.59-4.37 (m, 1H), 3.60 (s, 1H), 3.52 (s, 1H), 2.62 (s, 3H), 2.55 (d, J=4.6 Hz, 3H), 2.17 (d, J=11.7 Hz, 1H), 1.88 (s, 1H), 1.58 (d, J=6.9 Hz, 6H), 1.31 (ddt, J=35.9, 25.6, 13.1 Hz, 4H).

### Example 9

### 6-(5-chloro-2-(((3S,4R)-3-hydroxytetrahydro-2H-pyran-4-yl)amino)pyrimidin-4-yl)-4,4-dimethyl-3,4-dihydroisoquinolin-1(2H)-one (Compound I-116)

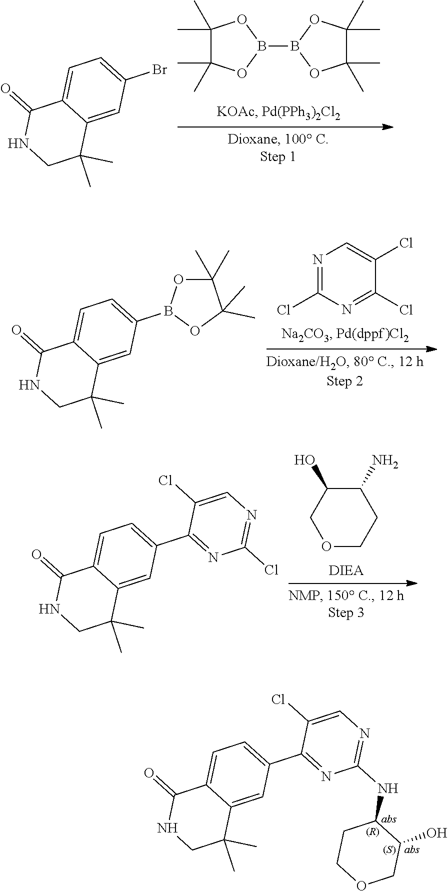

### 4,4-dimethyl-6-(4,4,5,5-tetramethyl-1,3,2-dioxaborolan-2-yl)-3,4-dihydroisoquinolin-1(2H)-one

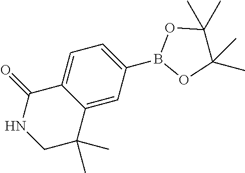 [SMILES: CC1(C)CNC(=O)c2ccc(B3OC(C)(C)C(C)(C)O3)cc21]

Step 1. To a solution of 6-bromo-4,4-dimethyl-3,4-dihydroisoquinolin-1(2H)-one (300 mg, 1.18 mmol) and bis(pinacolato)diboron (600 mg, 2.36 mmol) in 1,4-dioxane (3 mL) were added KOAc (348 mg, 3.54 mmol) and Pd(pph₃)₂Cl₂ (83 mg, 0.12 mmol), and the resulting mixture was stirred at 100° C. under a nitrogen atmosphere for 12 hours. The solution was purified by reverse-phase flash chromatography (column.: C18 silica gel; mobile phase: ACN in water, gradient: 10% to 50% in 20 min; wavelength: 254 nm). The desired fractions were combined, and then concentrated in vacuo to afford 4,4-dimethyl-6-(4,4,5,5-tetramethyl-1,3,2-dioxaborolan-2-yl)-3,4-dihydroisoquinolin-1(2H)-one (124 mg, 30%) as a black oil. m/z (ES⁺) [M+H]⁺=302.25; HPLC tR=1.018 min.

### 6-(2,5-dichloropyrimidin-4-yl)-4,4-dimethyl-3,4-dihydroisoquinolin-1(2H)-one

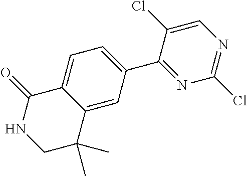 [SMILES: CC1(C)CNC(=O)c2ccc(-c3nc(Cl)ncc3Cl)cc21]

Step 2. To a solution of 4,4-dimethyl-6-(4,4,5,5-tetramethyl-1,3,2-dioxaborolan-2-yl)-3,4-dihydroisoquinolin-1(2H)-one (124 mg, 0.41 mmol) and 2,4,5-trichloropyrimidine (151 mg, 0.82 mmol) in 1,4-dioxane (2 mL) and H₂O (0.4 mL) were added Na₂CO₃ (131 mg, 1.24 mmol) and Pd(dppf)Cl₂ (30 mg, 0.04 mmol), and the resulting mixture was stirred at 80° C. under a nitrogen atmosphere for 12 hours. The solution was purified by reverse-phase flash chromatography (column: C18 silica gel; mobile phase, ACN in water, 0% to 100% gradient in 20 min; wavelength: 254 nm). The desired fractions were combined, and then concentrated in vacuo to afford 6-(2,5-dichloropyrimidin-4-yl)-4,4-dimethyl-3,4-dihydroisoquinolin-1(2H)-one (104 mg, 63%) as a black solid. m/z (ES⁺) [M+H]⁺=321.95; HPLC tR=0.858 min

### 6-(5-chloro-2-(((3S,4R)-3-hydroxytetrahydro-2H-pyran-4-yl)amino)pyrimidin-4-yl)-4,4-dimethyl-3,4-dihydroisoquinolin-1(2H)-one (Compound I-116)

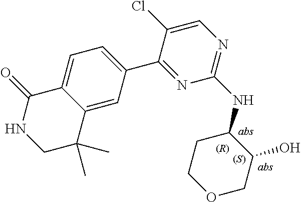

Step 3. A round-bottom flask was charged with 6-(2,5-dichloropyrimidin-4-yl)-4,4-dimethyl-3,4-dihydroisoquinolin-1(2H)-one (99 mg, 0.31 mmol), (3S,4R)-4-aminotetrahydro-2H-pyran-3-ol hydrochloride (57 mg, 0.37 mmol) and DIEA (120 mg, 0.92 mmol) in NMP (1 mL), and a stir bar, and the resulting solution was stirred at 150° C. for 12 hours. Then, the solution was purified by reverse-phase flash chromatography (column: C18 silica gel; mobile phase: ACN in water; gradient: 0% to 100% in 25 min; wavelength: 254 nm). The desired fractions were combined, and then concentrated in vacuo to afford 6-(5-chloro-2-(((3S,4R)-3-hydroxytetrahydro-2H-pyran-4-yl)amino)pyrimidin-4-yl)-4,4-dimethyl-3,4-dihydroisoquinolin-1(2H)-one (65.4 mg, 51%) as a yellow amorphous solid. m/z (ES⁺) [M+H]⁺=403.10; HPLC tR=1.318 min ¹H NMR (400 MHz, DMSO-d6) 8.43 (s, 1H), 8.09 (s, 1H), 7.96 (d, J=8.0 Hz, 1H), 7.70 (s, 2H), 7.51 (s, 1H), 4.92 (d, J=5.4 Hz, 1H), 3.81 (q, J=5.4 Hz, 3H), 3.51 (d, J=14.6 Hz, 1H), 3.31 (s, 1H), 3.23 (d, J=3.0 Hz, 2H), 3.02 (t, J=10.4 Hz, 1H), 1.92 (d, J=13.1 Hz, 1H), 1.59-1.44 (m, 1H), 1.37-1.21 (m, 6H).

### Example 10

### (1S,2S,5S)-2-((5-chloro-4-(4-fluoro-1-isopropyl-2-methyl-1H-benzo[d]imidazol-6-yl)pyrimidin-2-yl)amino)-5-isopropoxycyclohexan-1-ol (Compound I-117)

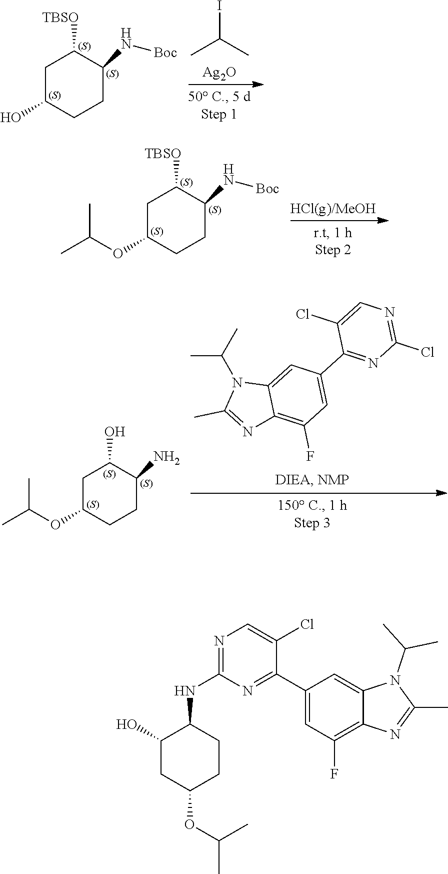

### tert-butyl ((1S,2S,4S)-2-((tert-butyldimethylsilyl)oxy)-4-isopropoxycyclohexyl)carbamate

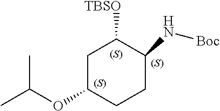

Step 1. A resealable reaction vial was charged with tert-butyl ((1S,2S,4S)-2-((tert-butyldimethylsilyl)oxy)-4-hydroxycyclohexyl)carbamate (400 mg, 1.16 mmol), Ag₂O (537 mg, 2.32 mmol), 2-iodopropane (5 mL) and a stir bar, and then evacuated and purged with nitrogen three times, and the resulting mixture was stirred at 50° C. for 5 days. The resulting crude material was purified by preparative HPLC (column: XBridge Shield RP18 OBD Column, 30×150 mm, 5 μm; mobile phase A: water (10 mM NH₄HCO₃+0.1% NH₃·H₂O); mobile phase B: ACN; flow rate: 60 mL/min; gradient: 57% B to 80% B in 8 min, 80% B; wavelength: 220 nm & 254 nm; tR1 (min): 7.17/7.67; injection volume: 0.6 mL; number of runs: 6). The desired fractions were combined, and then concentrated in vacuo to afford tert-butyl ((1S,2S,4S)-2-((tert-butyldimethylsilyl)oxy)-4-isopropoxycyclohexyl)carbamate (80 mg, 18%) as an oil. m/z (ES⁺) [M+H]⁺=388.35; HPLC tR=1.310 min.

### (1S,2S,5S)-2-amino-5-isopropoxycyclohexan-1-ol

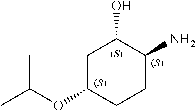

Step 2. A resealable reaction vial was charged with tert-butyl ((1S,2S,4S)-2-((tert-butyldimethylsilyl)oxy)-4-isopropoxycyclohexyl)carbamate (80 mg, 0.21 mmol), a mixture of MeOH and 4 M HCl in dioxane (1 mL) (1:1), and a stir bar, and then evacuated and purged with nitrogen three times, and the resulting mixture was stirred at 25° C. for 1 hour. Concentration in vacuo afforded (1S,2S,5S)-2-amino-5-isopropoxycyclohexan-1-ol (30 mg, 84%) as a white solid. m/z (ES⁺) [M+H]⁺=174.20; HPLC tR=0.185 min.

### (1S,2S,5S)-2-((5-chloro-4-(4-fluoro-1-isopropyl-2-methyl-1H-benzo[d]imidazol-6-yl)pyrimidin-2-yl)amino)-5-isopropoxycyclohexan-1-ol

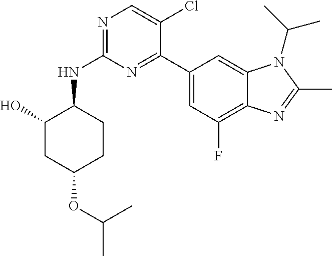 [SMILES: Cc1nc2c(F)cc(-c3nc(N[C@H]4CC[C@H](OC(C)C)C[C@@H]4O)ncc3Cl)cc2n1C(C)C]

Step 3. A resealable reaction vial was charged with (1S,2S,5S)-2-amino-5-isopropoxycyclohexan-1-ol (50 mg, 0.29 mmol), 6-(2,5-dichloropyrimidin-4-yl)-4-fluoro-1-isopropyl-2-methyl-1H-benzo[d]imidazole (0.10 g, 0.30 mmol), DIEA (0.22 g, 1.7 mmol), NMP (3 mL) and a stir bar, and then evacuated and purged with nitrogen three times, and the resulting mixture was stirred at 150° C. for 1 hour. The residue was purified by reverse-phase flash chromatography (column: C18 silica gel; mobile phase: ACN in water; gradient: 10% to 50% in 20 min; wavelength: 254 nm). The desired fractions were combined, and then concentrated in vacuo to afford a crude material, which was then purified by preparative HPLC (column: XBridge Prep OBD C18 Column, 30×150 mm, 5 μm; mobile phase A: water (10 mM NH₄HCO₃+0.1% NH₃·H₂O); mobile phase B: ACN; flow rate: 60 mL/min; gradient: 28% B to 57% B in 8 min, 57% B; wavelength: 220 nm; tR1 (min): 7.57; injection volume: 0.07 mL; number of runs: 3). The desired fractions were combined, and then lyophilized to afford (1S,2S,5S)-2-((5-chloro-4-(4-fluoro-1-isopropyl-2-methyl-1H-benzo[d]imidazol-6-yl)pyrimidin-2-yl)amino)-5-isopropoxycyclohexan-1-ol (100.2 mg, 73%) as a white amorphous solid. m/z (ES⁺) [M+H]⁺=476.10; HPLC tR=1.062 min ¹H NMR (400 MHz, DMSO-d₆) δ 8.39 (s, 1H), 7.92 (s, 1H), 7.38 (s, 1H), 7.25 (s, 1H), 4.81 (p, J=6.9 Hz, 1H), 4.69 (d, J=5.2 Hz, 1H), 3.68 (p, J=6.1 Hz, 1H), 3.56 (d, J=8.9 Hz, 1H), 3.45 (s, 1H), 3.30 (s, 1H), 2.62 (s, 3H), 2.14 (d, J=12.2 Hz, 1H), 2.08-1.74 (m, 2H), 1.58 (d, J=6.9 Hz, 6H), 1.28-1.11 (m, 3H), 1.06 (dd, J=6.1, 1.0 Hz, 6H).

### Example 11

### (3S,4R)-4-((4-(3-(tert-butyl)imidazo[1,2-a]pyridin-6-yl)-5-chloropyrimidin-2-yl)amino)tetrahydro-2H-pyran-3-ol Compound (I-158)

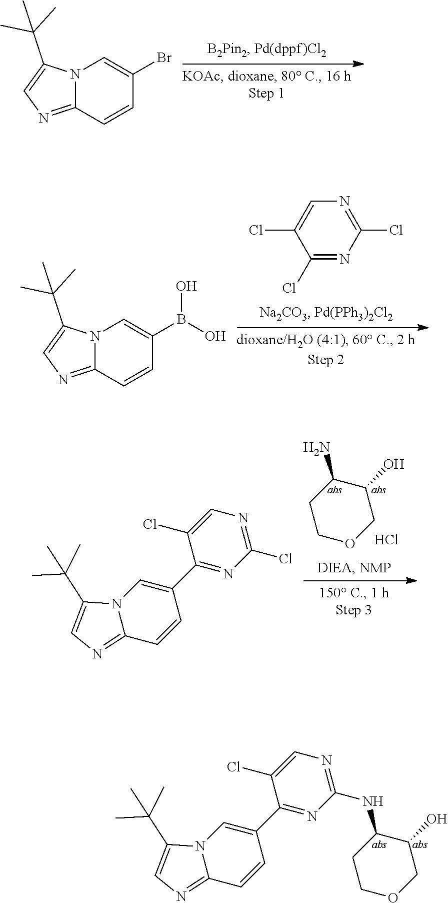

### (3-(tert-butyl)imidazo[1,2-a]pyridin-6-yl)boronic acid

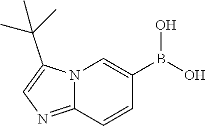 [SMILES: CC(C)(C)c1cnc2ccc(B(O)O)cn12]

Step 1. A resealable reaction vial was charged with 6-bromo-3-(tert-butyl)imidazo[1,2-a]pyridine (100 mg, 0.40 mmol) and bis(pinacolato)diboron (120 mg, 0.47 mmol) in 1,4-dioxane (4 mL), and a stir bar. Next were added potassium acetate (116 mg, 1.19 mmol) and PdCl₂(dppf) (28.9 mg, 0.04 mmol), and the resulting mixture was evacuated and purged with nitrogen three times, and then stirred at 80° C. under nitrogen atmosphere for 16 hours. The crude material was filtered through a pad of Celite, the pad was washed with 1,4-dioxane, and the filtrate was concentrated in vacuo to provide the title compound, which was used for the next step without any further purification. m/z (ES⁺) [M+H]⁺=219.05; HPLC tR=0.385 min.

### 3-(tert-butyl)-6-(2,5-dichloropyrimidin-4-yl)imidazo[1,2-a]pyridine

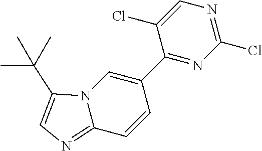 [SMILES: CC(C)(C)c1cnc2ccc(-c3nc(Cl)ncc3Cl)cn12]

Step 2. A resealable reaction vial was charged with crude (3-(tert-butyl)imidazo[1,2-a]pyridin-6-yl)boronic acid (86.5 mg, 0.40 mmol), 2,4,5-trichloropyrimidine (84.1 mg, 0.46 mmol), Na₂CO₃ (146 mg, 1.38 mmol) and Pd(PPh₃)₂Cl₂ (28 mg, 0.04 mmol), dioxane (4 mL), H₂O (1 mL) and a stir bar, and then evacuated and purged with nitrogen three times. The resulting mixture was stirred at 60° C. for 2 hours. The resulting crude material was purified by flash chromatography (mobile phase: acetonitrile/water; gradient: 50% to 90% in 15 min; wavelength: 254 nm). The desired fractions were combined, and then concentrated in vacuo to afford 3-(tert-butyl)-6-(2,5-dichloropyrimidin-4-yl)imidazo[1,2-a]pyridine (35 mg, 24%) as a yellow amorphous solid. m/z (ES⁺) [M+H]⁺=321.10; HPLC tR=0.910 min.

### (3S,4R)-4-((4-(3-(tert-butyl)imidazo[1,2-a]pyridin-6-yl)-5-chloropyrimidin-2-yl)amino)tetrahydro-2H-pyran-3-ol

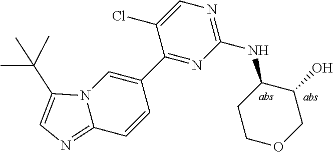 [SMILES: CC(C)(C)c1cnc2ccc(-c3nc(N[C@@H]4CCOC[C@H]4O)ncc3Cl)cn12]

Step 3. A resealable reaction vial was charged with 3-(tert-butyl)-6-(2,5-dichloropyrimidin-4-yl)imidazo[1,2-a]pyridine (30 mg, 0.09 mmol) and (3S,4R)-4-aminotetrahydro-2H-pyran-3-ol hydrochloride (14 mg, 0.09 mmol) in NMP (1 mL), a stir bar, and DIEA (60 mg, 0.47 mmol), and the resulting mixture was evacuated and purged with nitrogen three times, and then stirred at 150° C. under nitrogen atmosphere for 1 hour. The resulting crude material was purified by preparative HPLC (column: XBridge Prep OBD C18 Column, 30×150 mm, 5 μm; mobile phase A: mmol NH₄HCO₃+0.05% NH₃H₂O; mobile phase B: ACN; flow rate: 60 mL/min; gradient: 24% B to 49% B in 8 min, 49% B; wavelength: 254 nm; tR1 (min): 7.48; injection volume: 0.8 mL; number of runs: 2). The desired fractions were combined, and then lyophilized to afford (3S,4R)-4-((4-(3-(tert-butyl)imidazo[1,2-a]pyridin-6-yl)-5-chloropyrimidin-2-yl)amino)tetrahydro-2H-pyran-3-ol (5.3 mg, 14%) as an off-white solid. m/z (ES⁺) [M+H]⁺=402.10; HPLC tR=0.972 min. ¹H NMR (400 MHz, DMSO-d6) 8.99 (s, 1H), 8.44 (s, 1H), 7.71-7.52 (m, 3H), 7.44 (s, 1H), 4.96 (d, J=5.3 Hz, 1H), 3.89-3.77 (m, 3H), 3.51 (s, 1H), 3.27 (s, 1H), 3.02 (t, J=10.3 Hz, 1H), 1.95 (s, 1H), 1.48 (s, 10H).

### Example 12

### (4S,5S)-4-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-5-ol and (4R,5R)-4-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-5-ol (Compounds I-182 and I-183)

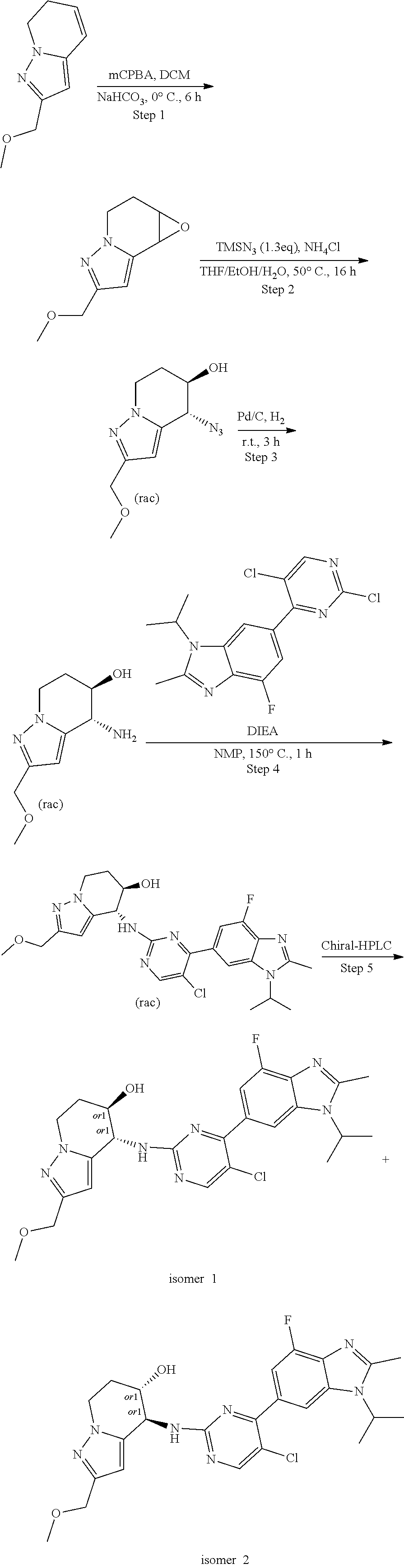

### 6-(methoxymethyl)-1a,2,3,7b-tetrahydrooxireno[2,3-c]pyrazolo[1,5-a]pyridine

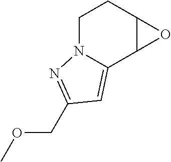

Step 1. To a mixture of 2-(methoxymethyl)-6,7-dihydropyrazolo[1,5-a]pyridine (260 mg, 1.58 mmol) in dichloromethane (5 mL) were added m-CPBA (820 mg, 4.75 mmol) and sodium bicarbonate (532 mg, 6.33 mmol) in portions at 0° C. under nitrogen atmosphere, and the resulting mixture was stirred at 0° C. for 6 hours. The crude residue was purified by silica-gel chromatography (60 g column; eluting with PE/EtOAc [4:5]). Concentration in vacuo afforded 6-(methoxymethyl)-1a,2,3,7b-tetrahydrooxireno[2,3-c]pyrazolo[1,5-a]pyridine (100 mg, 0.50 mmol, 32%, 90% purity) as a white solid. m/z (ES⁺) [M+H]⁺=181.10; HPLC tR=0.438 min.

### rac-(4R,5R)-4-azido-2-(methoxymethyl)-4,5,6,7-tetrahydropyrazolo[1,5-a]pyridin-5-ol

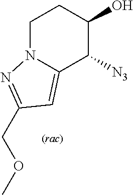 [SMILES: COCc1cc2n(n1)CC[C@@H](O)[C@@H]2N=[N+]=[N-]]

Step 2. To a solution of 6-(methoxymethyl)-1a,2,3,7b-tetrahydrooxireno[2,3-c]pyrazolo[1,5-a]pyridine (100 mg, 555 mol) in THF/EtOH/H₂O (1:2:0.25, 1.325 mL) were added azidotrimethylsilane (83.1 mg, 721 mol) and ammonium chloride (47.5 mg, 888 mol), and the resulting mixture was heated at 50° C. overnight. After cooling, the residue was purified by reverse-phase flash chromatography (column: C18 silica gel; mobile phase, acetonitrile in water, 10% to 50% gradient in 10 min; wavelength: 254 nm). The desired fractions were combined, and then concentrated in vacuo to afford rac-(4R,5R)-4-azido-2-(methoxymethyl)-4,5,6,7-tetrahydropyrazolo[1,5-a]pyridin-5-ol (40 mg, 0.14 mmol, 26%, 80% purity) as a white solid. m/z (ES⁺) [M+H]⁺=224.15; HPLC tR=0.843 min.

### rac-(4R,5R)-4-amino-2-(methoxymethyl)-4,5,6,7-tetrahydropyrazolo[1,5-a]pyridin-5-ol

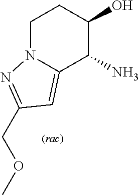

Step 3. A stirred mixture of rac-(4R,5R)-4-azido-2-(methoxymethyl)-4,5,6,7-tetrahydropyrazolo[1,5-a]pyridin-5-ol (40 mg, 0.18 mmol) and Pd/C (4.0 mg, 38 mol) in MeOH (5 mL) was treated with H₂ at 25° C. for 3 hours. The reaction mixture was filtered, and the filtrate was concentrated to afford rac-(4R,5R)-4-amino-2-(methoxymethyl)-4,5,6,7-tetrahydropyrazolo[1,5-a]pyridin-5-ol (60 mg, 0.15 mmol, 85%, 50% purity) as white solid. m/z (ES⁺) [M+H]⁺=198.10; HPLC tR=0.298 min.

### rac-((4R,5R)-4-((5-chloro-4-(4-fluoro-1-isopropyl-2-methyl-1H-benzo[d]imidazol-6-yl) pyrimidin-2-yl) amino)-2-(methoxymethyl)-4,5,6,7-tetrahydropyrazolo [1,5-a]pyridin-5-ol

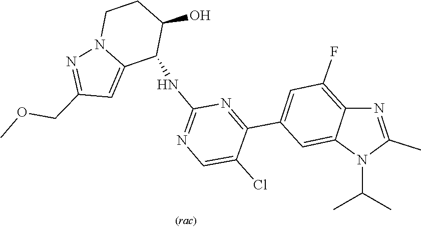

Step 4. To a solution of rac-(4R,5R)-4-amino-2-(methoxymethyl)-4,5,6,7-tetrahydropyrazolo[1,5-a]pyridin-5-ol (60 mg, 0.30 mmol) and 6-(2,5-dichloropyrimidin-4-yl)-4-fluoro-1-isopropyl-2-methyl-1H-benzo[d]imidazole (93.0 mg, 0.27 mmol) in NMP (1 mL) was added DIEA (39.0 mg, 53 μL, 0.30 mmol) at r.t. The solution was stirred at 150° C. for 1 hour. The mixture was cooled to r.t., and then purified by reverse-phase flash chromatography (column: C18 silica gel; mobile phase, acetonitrile in water, 10% to 50% gradient in 10 min; wavelength: 254 nm). The desired fractions were combined, and then concentrated in vacuo to afford rac-((4R,5R)-4-((5-chloro-4-(4-fluoro-1-isopropyl-2-methyl-1H-benzo[d]imidazol-6-yl) pyrimidin-2-yl) amino)-2-(methoxymethyl)-4,5,6,7-tetrahydropyrazolo [1,5-a]pyridin-5-ol (40 mg, 76 mol, 25%, 95% purity)) as a white solid. m/z (ES⁺) [M+H]⁺=500.05; HPLC tR=0.850 min.

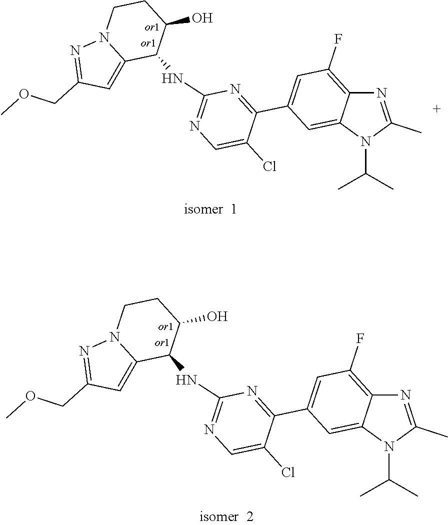

Step 5. The crude product obtained in Step 4 (50 mg) was purified by prep chiral HPLC (column: CHIRALPAK IG, 2×25 cm, 5 μm; mobile phase A: Hex (0.2% TEA) HPLC; mobile phase B: EtOH/DCM (1:1) HPLC; flow rate: 20 mL/min; gradient: isocratic; wavelength: 220 nm & 254 nm; tR1 (min): 7.97; sample solvent: EtOH/DCM (1:1) HPLC; injection volume: 0.7 mL; number of runs: 3) to afford rel-(4R,5R)-4-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-5-ol (15.2 mg, 30.3 μmol, 61%, 99.7% purity) as an off-white amorphous solid: m/z (ES⁺) [M+H]⁺=500.10; HPLC tR=0.883 min. ¹H NMR (400 MHz, DMSO-d6) 8.47 (s, 1H), 7.93 (d, J=8.0 Hz, 2H), 7.37 (d, J=11.7 Hz, 1H), 6.03 (s, 1H), 5.32 (s, 1H), 5.09 (s, 1H), 4.80 (p, J=6.9 Hz, 1H), 4.25 (s, 2H), 4.10 (q, J=6.2, 5.7 Hz, 3H), 3.21 (s, 3H), 2.62 (s, 3H), 2.32 (s, 1H), 2.02 (dt, J=14.2, 7.0 Hz, 1H), 1.57 (m, 6H) and rel-(4S,5S)-4-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-5-ol (15.6 mg, 30.8 mol, 62%, 98.7% purity) as an off-white amorphous solid: m/z (ES⁺) [M+H]⁺=500.10; HPLC tR=1.438 min. ¹H NMR (400 MHz, DMSO-d6) 8.49 (s, 1H), 7.93 (d, J=7.9 Hz, 2H), 7.37 (d, J=11.7 Hz, 1H), 6.03 (s, 1H), 5.32 (s, 1H), 5.09 (s, 1H), 4.80 (p, J=7.0 Hz, 1H), 4.25 (s, 2H), 4.10 (m, 3H), 3.21 (s, 3H), 2.62 (s, 3H), 2.32 (m, 1H), 2.02 (dt, J=14.2, 7.0 Hz, 1H), 1.57 (d, J=6.8 Hz, 6H).

### Example 13

### 6-(5-chloro-2-(((3S,4R)-3-hydroxytetrahydro-2H-pyran-4-yl)amino)pyrimidin-4-yl)-8-fluoro-4,4-dimethyl-3,4-dihydroisoquinolin-1(2H)-one (Compound I-201)

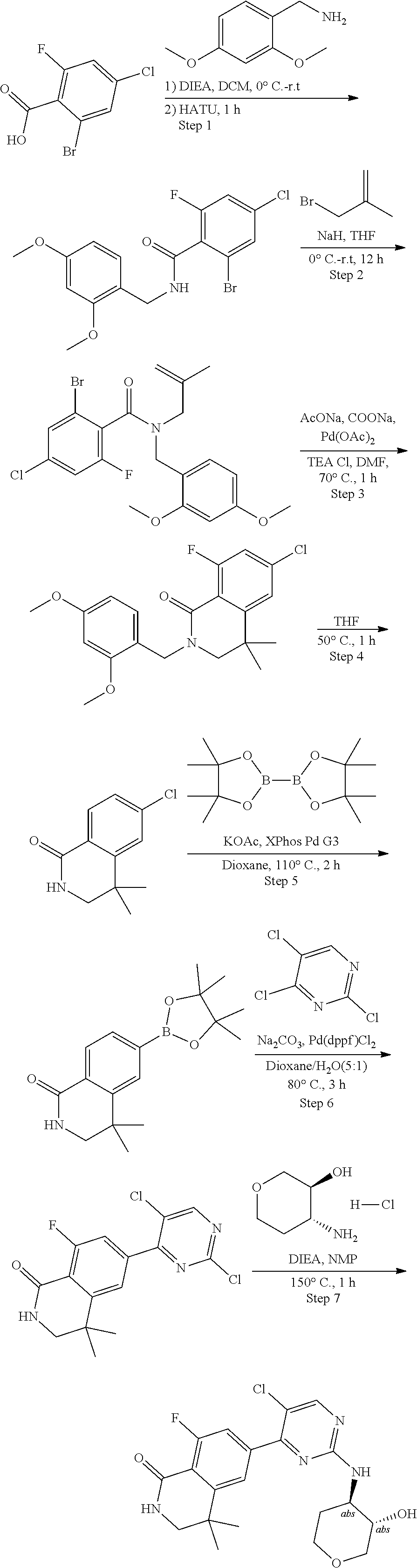

### 2-bromo-4-chloro-N-(2,4-dimethoxybenzyl)-6-fluorobenzamide

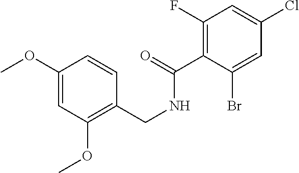 [SMILES: COc1ccc(CNC(=O)c2c(F)cc(Cl)cc2Br)c(OC)c1]

Step 1. To a mixture of 2-bromo-4-chloro-6-fluorobenzoic acid (2.00 g, 8.00 mmol) and DIEA (1.86 g, 14.4 mmol) in DCM (10 mL) was added HATU (2.73 g, 7.18 mmol) in portions at 25° C. under nitrogen atmosphere, and the resulting mixture was stirred at 25° C. for 30 min. Then, (2,4-dimethoxyphenyl)methanamine (0.80 g, 4.78 mmol) in DCM was added, and the reaction mixture was stirred at 25° C. for an additional 1 hour. The crude mixture was diluted with H₂O (15 mL), and the aq. phase was extracted with EtOAc (3×30 mL). The combined organic layers were washed with brine, dried over sodium sulfate, filtered, and then concentrated in vacuo. The residue was purified by reverse-phase flash chromatography (column: C18 silica gel; mobile phase, ACN in water; gradient: 0% to 100% in 20 min; wavelength: 254 nm). The desired fractions were combined, and then concentrated in vacuo to afford 2-bromo-4-chloro-N-(2,4-dimethoxybenzyl)-6-fluorobenzamide (1.15 g, 59.7%) as a colorless oil. m/z (ES⁺) [M+H]⁺=402.15; HPLC tR=1.149 min.

### 2-bromo-4-chloro-N-(2,4-dimethoxybenzyl)-6-fluoro-N-(2-methylallyl)benzamide

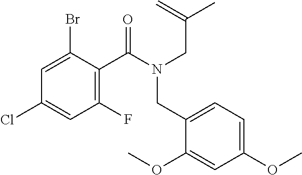 [SMILES: C=C(C)CN(Cc1ccc(OC)cc1OC)C(=O)c1c(F)cc(Cl)cc1Br]

Step 2. To a solution of 2-bromo-4-chloro-N-(2,4-dimethoxybenzyl)-6-fluorobenzamide (1.15 g, 2.86 mmol) in THF (15 mL) was added NaH (0.34 g, 60% Wt, 8.57 mmol) in portions at 0° C., and the resulting mixture was stirred for 30 min. Then, 3-bromo-2-methylprop-1-ene (0.46 g, 3.43 mmol) was added, and the reaction mixture was allowed to warm to 25° C., and then stirred for 12 hours. The crude mixture was diluted with ice water (15 mL), and the aq. phase was extracted with EtOAc (3×30 mL). The combined organic layers were washed with brine, dried over sodium sulfate, filtered, and then concentrated in vacuo. The residue was purified by reverse-phase flash chromatography (column: C18 silica gel; mobile phase, ACN in water; gradient: 0% to 100% in 20 min; wavelength: 254 nm). The desired fractions were combined, and then concentrated in vacuo to afford 2-bromo-4-chloro-N-(2,4-dimethoxybenzyl)-6-fluoro-N-(2-methylallyl)benzamide (0.86 g, 65.9%) as a colorless oil. m/z (ES⁺) [M+H]⁺=456.10; HPLC tR=1.268 min.

### 6-chloro-2-(3,5-dimethoxybenzyl)-8-fluoro-4,4-dimethyl-3,4-dihydroisoquinolin-1(2H)-one

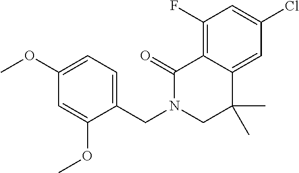 [SMILES: COc1ccc(CN2CC(C)(C)c3cc(Cl)cc(F)c3C2=O)c(OC)c1]

Step 3. To a solution of 2-bromo-4-chloro-N-(3,5-dimethoxybenzyl)-6-fluoro-N-(2-methylallyl)benzamide (1.60 g, 3.5 mmol) and sodium formate (0.26 g, 3.9 mmol) in DMF (4 mL), were added sodium acetate (0.63 g, 7.7 mmol), tetraethylammonium chloride (0.58 g, 3.5 mmol) and palladium diacetate (0.08 g, 0.35 mmol), and the resulting mixture was stirred at 70° C. under a nitrogen atmosphere for 1 hour. The reaction mixture was diluted with H₂O (15 mL), and the aq. phase was extracted with EtOAc (3×30 mL). The combined organic layers were washed with brine, dried over sodium sulfate, filtered, and then concentrated in vacuo. The residue was purified by preparative TLC, eluting with PE/EtOAc (4:1). The desired fractions were combined, and then concentrated in vacuo to afford 6-chloro-2-(3,5-dimethoxybenzyl)-8-fluoro-4,4-dimethyl-3,4-dihydroisoquinolin-1(2H)-one (1.5 g, 99%) as a yellow oil. m/z (ES⁺) [M+H]⁺=378.15; HPLC tR=1.048 min.

### 6-chloro-8-fluoro-4,4-dimethyl-3,4-dihydroisoquinolin-1(2H)-one

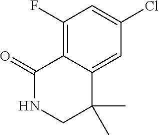

Step 4. A round-bottom flask was charged with 6-chloro-2-(2,4-dimethoxybenzyl)-8-fluoro-4,4-dimethyl-3,4-dihydroisoquinolin-1(2H)-one (460 mg, 1.22 mmol) in TFA (2 mL) and a stir bar, the solution was stirred at 50° C. for 1 hour. The mixture was quenched with sat'd. NaHCO₃ (aq.) and diluted with H₂O (15 mL), and then the aq. phase was extracted with EtOAc (3×30 mL). The combined organic layers were washed with brine, dried over sodium sulfate, filtered, and then concentrated in vacuo. The residue was purified by reverse-phase flash chromatography (column: C18 silica gel; mobile phase, ACN in water; gradient: 0% to 60% in 20 min; wavelength: 254 nm). The desired fractions were combined, and then concentrated in vacuo to afford 6-chloro-8-fluoro-4,4-dimethyl-3,4-dihydroisoquinolin-1(2H)-one (250 mg, 86%) as a white solid. m/z (ES⁺) [M+H]⁺=228.10; HPLC tR=0.675 min.

### 8-fluoro-4,4-dimethyl-6-(4,4,5,5-tetramethyl-1,3,2-dioxaborolan-2-yl)-3,4-dihydroisoquinolin-1(2H)-one

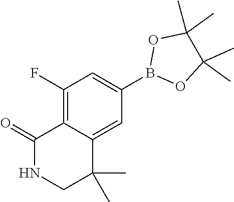 [SMILES: CC1(C)CNC(=O)c2c(F)cc(B3OC(C)(C)C(C)(C)O3)cc21]

Step 5. To a solution of 6-chloro-8-fluoro-4,4-dimethyl-3,4-dihydroisoquinolin-1(2H)-one (100 mg, 0.44 mmol) and B₂Pin₂ (223 mg, 0.88 mmol) in 1,4-dioxane (1 mL) were added KOAc (129 mg, 1.32 mmol) and XPhos Pd G3 (37.2 mg, 0.04 mmol), and the resulting mixture was stirred at 110° C. under a nitrogen atmosphere for 2 hours. Then, the mixture was diluted with H₂O (15 mL), and the aq. phase was extracted with EtOAc (3×30 mL). The combined organic layers were washed with brine, dried over sodium sulfate, filtered, and then concentrated in vacuo to afford crude 8-fluoro-4,4-dimethyl-6-(4,4,5,5-tetramethyl-1,3,2-dioxaborolan-2-yl)-3,4-dihydroisoquinolin-1(2H)-one (210 mg, 150%) as a black oil. m/z (ES⁺) [M+H]⁺=320.25; HPLC tR=1.135 min.

### 6-(2,5-dichloropyrimidin-4-yl)-8-fluoro-4,4-dimethyl-3,4-dihydroisoquinolin-1(2H)-one

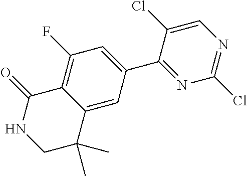 [SMILES: CC1(C)CNC(=O)c2c(F)cc(-c3nc(Cl)ncc3Cl)cc21]

Step 6. To a solution of 8-fluoro-4,4-dimethyl-6-(4,4,5,5-tetramethyl-1,3,2-dioxaborolan-2-yl)-3,4-dihydroisoquinolin-1(2H)-one (205 mg, 0.64 mmol) and 2,4,5-trichloropyrimidine (130 mg, 0.71 mmol) in 1,4-dioxane (2 mL) and H₂O (0.4 mL) were added Na₂CO₃ (204 mg, 1.93 mmol) and Pd(dppf)Cl₂ (47 mg, 0.06 mmol), and the resulting mixture was stirred at 80° C. under a nitrogen atmosphere for 3 hours. Then, the mixture was diluted with H₂O (15 mL), and the aq. phase was extracted with EtOAc (3×30 mL). The combined organic layers were washed with brine, dried over sodium sulfate, filtered, and then concentrated in vacuo. The residue was purified by reverse-phase flash chromatography (column: C18 silica gel; mobile phase, ACN in water; gradient: 0% to 100% in 20 min; wavelength: 254 nm). The desired fractions were combined, and then concentrated in vacuo to afford 6-(2,5-dichloropyrimidin-4-yl)-8-fluoro-4,4-dimethyl-3,4-dihydroisoquinolin-1(2H)-one (60 mg, 14%) as a black solid. m/z (ES⁺) [M+H]⁺=340.00; HPLC tR=0.732 min.

### 6-(5-chloro-2-(((3S,4R)-3-hydroxytetrahydro-2H-pyran-4-yl)amino)pyrimidin-4-yl)-8-fluoro-4,4-dimethyl-3,4-dihydroisoquinolin-1(2H)-one (Compound I-201)

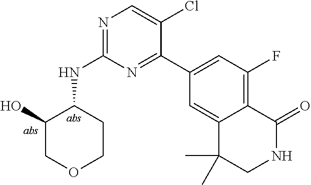 [SMILES: CC1(C)CNC(=O)c2c(F)cc(-c3nc(N[C@@H]4CCOC[C@H]4O)ncc3Cl)cc21]

Step 7. A round-bottom flask was charged with 6-(2,5-dichloropyrimidin-4-yl)-8-fluoro-4,4-dimethyl-3,4-dihydroisoquinolin-1(2H)-one (25 mg, 0.07 mmol), (3S,4R)-4-aminotetrahydro-2H-pyran-3-ol hydrochloride (12 mg, 0.08 mmol) and DIEA (28 mg, 0.22 mmol) in NMP (1 mL) and a stir bar, and the solution was stirred at 150° C. for 1 hour. The resulting mixture was diluted with H₂O (15 mL), and the aq. phase was extracted with EtOAc (3×30 mL). The combined organic layers were washed with brine, dried over sodium sulfate, filtered, and then concentrated in vacuo. The resulting crude material was purified by preparative HPLC (column: XBridge Shield RP18 OBD Column, 30×150 mm, 5 μm; mobile phase A: water (10 mM NH₄HCO₃+0.05% NH₃H₂O); mobile phase B: ACN; flow rate: 60 mL/min; gradient: 17% B to 38% B in 7 min, wavelength: 220 nm & 254 nm); tR1 (min): 7.0; number of runs: 2). The desired fractions were combined, and then lyophilized to afford 6-(5-chloro-2-(((3S,4R)-3-hydroxytetrahydro-2H-pyran-4-yl)amino)pyrimidin-4-yl)-8-fluoro-4,4-dimethyl-3,4-dihydroisoquinolin-1(2H)-one (11.9 mg, 38%) as a white amorphous solid. m/z (ES⁺) [M+H]⁺=421.05; HPLC tR=0.832 min. ¹H NMR (400 MHz, DMSO-d6) 8.44 (s, 1H), 8.15 (s, 1H), 7.50 (d, J=44.1 Hz, 3H), 4.91 (d, J=5.4 Hz, 1H), 3.81 (dd, J=11.9, 6.0 Hz, 3H), 3.49 (s, 1H), 3.29 (s, 1H), 3.17 (d, J=3.5 Hz, 2H), 3.03 (s, 1H), 1.93 (s, 1H), 1.55-1.39 (m, 1H), 1.29 (s, 6H).

### Example 14

### (4R,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol and (4S,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (Compounds I-213 and I-218)

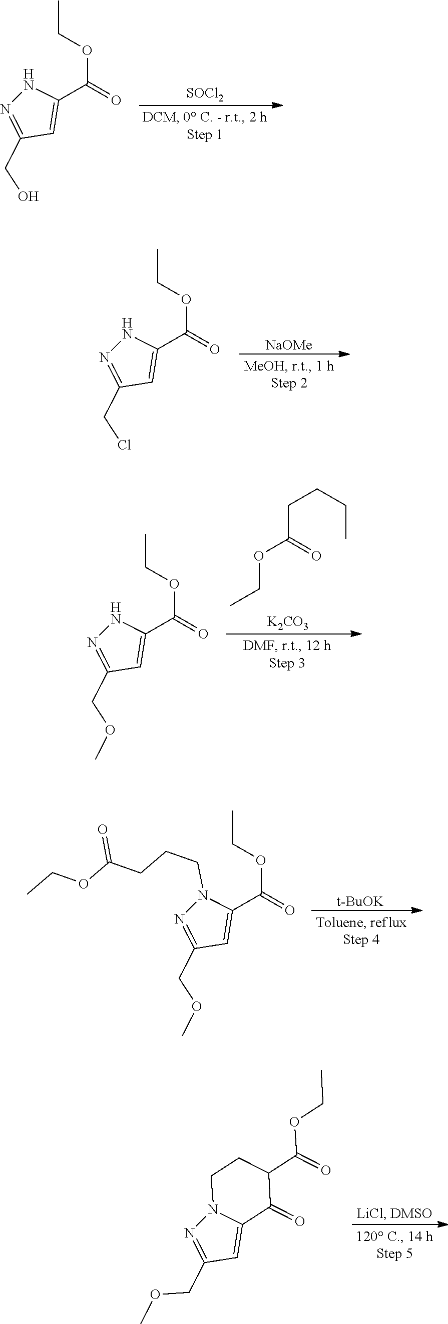

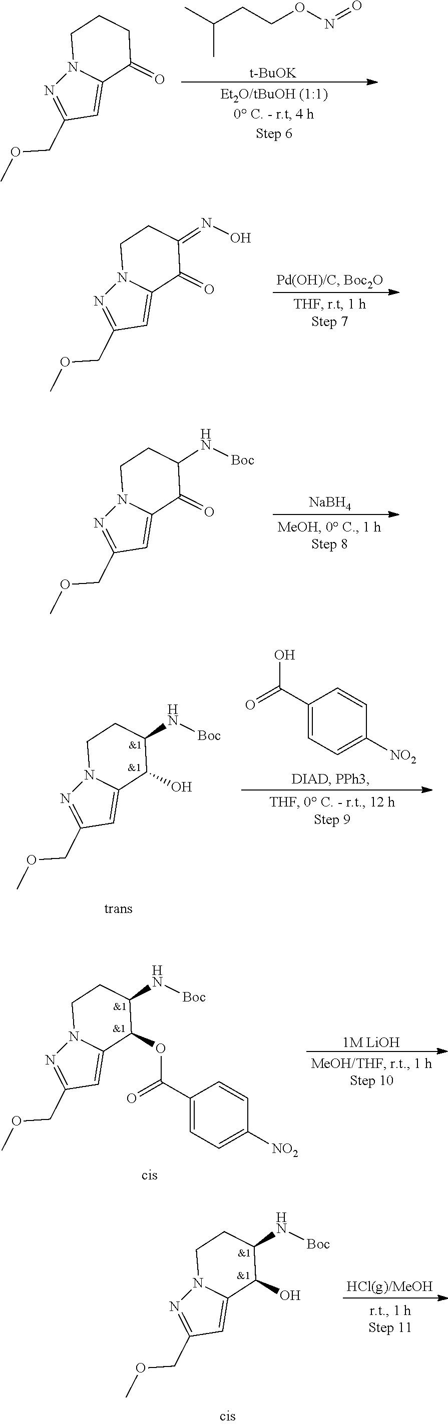

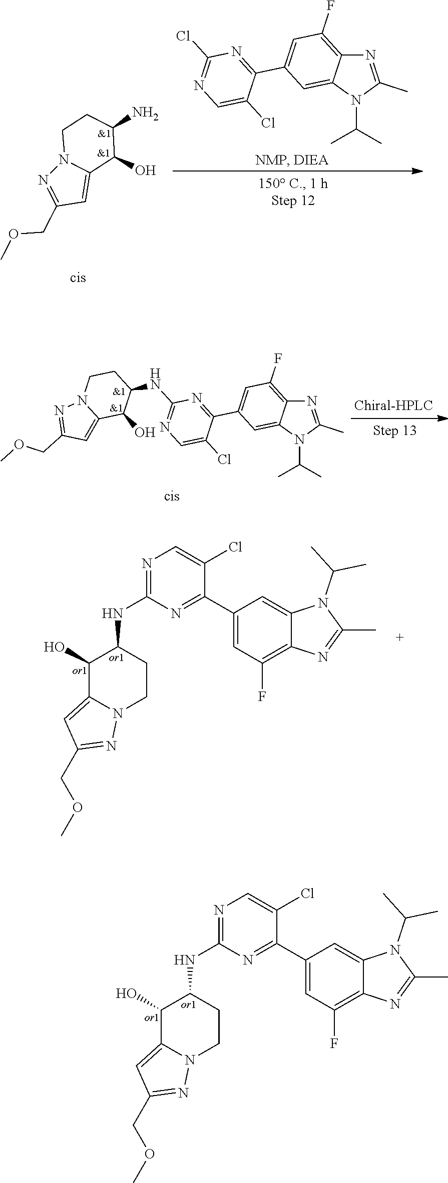

### ethyl 3-(chloromethyl)-1H-pyrazole-5-carboxylate

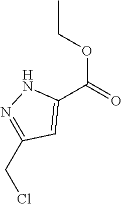

Step 1. To a mixture of ethyl 3-(hydroxymethyl)-1H-pyrazole-5-carboxylate (11.80 g, 69.30 mmol in DCM (150 mL) was added SOCl₂ (16.50 g, 139.00 mmol) dropwise at 0° C. under nitrogen atmosphere. The mixture was stirred at 25° C. for 2 hours, and then quenched with water (300 mL). The pH was adjusted to 6-7 with NaHCO₃ at 0° C. and the aq. phase was extracted with EtOAc (3×500 mL). The combined organic layers were dried over sodium sulfate, filtered, and then concentrated in vacuo to afford ethyl 3-(chloromethyl)-1H-pyrazole-5-carboxylate (15.00 g, 99%; crude) as a yellow oil. m/z (ES⁺) [M+H]⁺=189.10; HPLC tR=0.860 min.

### ethyl 3-(methoxymethyl)-1H-pyrazole-5-carboxylate

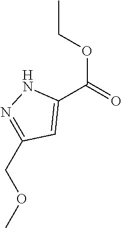

Step 2. To a mixture of ethyl 3-(chloromethyl)-1H-pyrazole-5-carboxylate (15.00 g, 80.00 mmol) in MeOH (180 mL) was added sodium methoxide (5.20 g, 95.00 mmol) in portions at 0° C. under nitrogen atmosphere. The mixture was stirred at 25° C. for 1 hour, and then quenched with water (50 mL). The pH was adjusted to 6-7 with sat'd. aq. NH₄Cl at 0° C., and the aq. phase was extracted with EtOAc (3×400 mL). The combined organic layers were dried over sodium sulfate, filtered, and then concentrated in vacuo. The crude product was purified by silica-gel chromatography, eluting with PE/EtOAc (3:1). The desired fractions were combined, and then concentrated in vacuo to afford ethyl 3-(methoxymethyl)-1H-pyrazole-5-carboxylate (13.50 g, 47%) (crude) as a yellow oil. m/z (ES⁺) [M+H]⁺=185.10; HPLC tR=0.572 min.

### ethyl 1-(4-ethoxy-4-oxobutyl)-3-(methoxymethyl)-1H-pyrazole-5-carboxylate

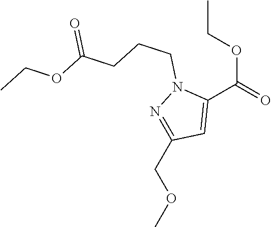 [SMILES: CCOC(=O)CCCn1nc(COC)cc1C(=O)OCC]

Step 3. A resealable reaction vial was charged with ethyl 3-(methoxymethyl)-1H-pyrazole-5-carboxylate (13.50 g, 73.30 mmol), ethyl 4-bromobutanoate (17.20 g, 88.00 mmol), K₂CO₃ (12.70 g, 91.60 mmol), DMF (160 mL) and a stir bar, and then evacuated and purged with nitrogen three times, and the resulting mixture was stirred at 25° C. for 12 hours. The mixture was diluted with water (600 mL) and the aq. phase was extracted with EtOAc (3×800 mL). The combined organic layers were washed with brine, dried over sodium sulfate, filtered, and then concentrated in vacuo. The crude product was purified by silica-gel chromatography, eluting with PE/EtOAc (3:1). The desired fractions were combined, and then concentrated in vacuo to afford ethyl 1-(4-ethoxy-4-oxobutyl)-3-(methoxymethyl)-1H-pyrazole-5-carboxylate (9.00 g, 40%) as a yellow oil. m/z (ES⁺) [M+H]⁺=299.10; HPLC tR=0.758 min.

### ethyl 2-(methoxymethyl)-4-oxo-4,5,6,7-tetrahydropyrazolo[1,5-a]pyridine-5-carboxylate

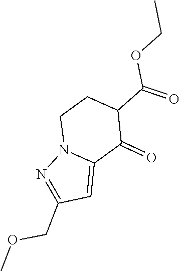 [SMILES: CCOC(=O)C1CCn2nc(COC)cc2C1=O]

Step 4. A resealable reaction vial was charged with ethyl 1-(4-ethoxy-4-oxobutyl)-3-(methoxymethyl)-1H-pyrazole-5-carboxylate (9.00 g, 0.03 mol), 2-methylpropan-2-olate potassium (5.00 g, 0.05 mol), toluene (100 mL) and a stir bar, and then evacuated and purged with nitrogen three times, and the resulting mixture was stirred for 30 min at 110° C. The reaction mixture was quenched with water, treated with sat'd. aq. NH₄Cl to adjust the pH to 6-7, and then the aq. phase was extracted with EtOAc (3×100 mL). The combined organic layers were dried over sodium sulfate, filtered, and then concentrated in vacuo to afford ethyl 2-(methoxymethyl)-4-oxo-4,5,6,7-tetrahydropyrazolo[1,5-a]pyridine-5-carboxylate (4.00 g, 50%) as a yellow oil. m/z (ES⁺) [M+H]⁺=253.05; HPLC tR=0.572 min.

### 2-(methoxymethyl)-6,7-dihydropyrazolo[1,5-a]pyridin-4 (5H)-one

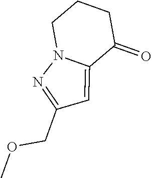

Step 5. A resealable reaction vial was charged with ethyl 2-(methoxymethyl)-4-oxo-4,5,6,7-tetrahydropyrazolo[1,5-a]pyridine-5-carboxylate (4.00 g, 0.02 mol), lithium chloride (1.00 g, 0.02 mol), DMSO (50 mL) and a stir bar, and then evacuated and purged with nitrogen three times, and the mixture was stirred at 120° C. for 14 hours. The resulting crude material was purified by flash chromatography (mobile phase: ACN/water; gradient: 0% to 30% in 15 min; wavelength: 254 nm). The desired fractions were combined, and then concentrated in vacuo to afford 2-(methoxymethyl)-6,7-dihydropyrazolo[1,5-a]pyridin-4 (5H)-one (1.90 g, 60%) as a black oil. m/z (ES⁺) [M+H]>=181.15; HPLC tR=0.318 min.

### (Z)-5-(hydroxyimino)-2-(methoxymethyl)-6,7-dihydropyrazolo[1,5-a]pyridin-4 (5H)-one

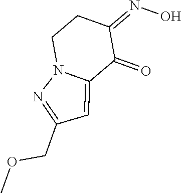

Step 6. A resealable reaction vial was charged with isopentyl nitrite (0.80 g, 7 mmol), 2-methylpropan-2-olate potassium (0.70 g, 7.00 mmol) in Et₂O (14 mL)/t-BuOH (14 mL), and a stir bar. Next, 2-(methoxymethyl)-6,7-dihydropyrazolo[1,5-a]pyridin-4 (5H)-one (1.00 g, 6.00 mmol) was added at 0° C., and the resulting mixture was stirred at 25° C. for 4 hours. The reaction mixture was quenched with water, the pH was adjusted to 5-7 with 1M HCl, and the aq. phase was extracted with EtOAc/THF (4:1; 3×80 mL). The combined organic layers were washed with brine, dried over sodium sulfate, filtered, and then concentrated in vacuo. The crude product was purified by silica-gel chromatography (120 g column), eluting with DCM/MeOH (10:1). The desired fractions were combined, and then concentrated in vacuo to afford (Z)-5-(hydroxyimino)-2-(methoxymethyl)-6,7-dihydropyrazolo[1,5-a]pyridin-4 (5H)-one (630 mg, 50%) as a yellow amorphous solid. m/z (ES⁺) [M+H]⁺=210.10; HPLC tR=0.461 min.

### tert-butyl (2-(methoxymethyl)-4-oxo-4,5,6,7-tetrahydropyrazolo[1,5-a]pyridin-5-yl)carbamate

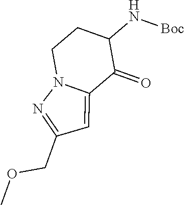 [SMILES: COCc1cc2n(n1)CCC(NC(=O)OC(C)(C)C)C2=O]

Step 7. A stirred mixture of (Z)-5-(hydroxyimino)-2-(methoxymethyl)-6,7-dihydropyrazolo[1,5-a]pyridin-4 (5H)-one (630 mg, 3.01 mmol), Boc₂O (131 mg, 602 μmol) and Pd(OH)₂/C (846 mg, 6.02 mmol) in THF (14 mL) was treated with H₂ at 25° C. for 1 hour. The reaction mixture was filtered through a pad of Celite, the pad was washed with EtOAc, and the filtrate was concentrated in vacuo to afford tert-butyl (2-(methoxymethyl)-4-oxo-4,5,6,7-tetrahydropyrazolo[1,5-a]pyridin-5-yl)carbamate (700 mg, 78.7%) as a yellow-green amorphous solid. m/z (ES⁺) [M+H]⁺=296.05; HPLC tR=0.903 min.

### rac-tert-butyl ((4S,5R)-4-hydroxy-2-(methoxymethyl)-4,5,6,7-tetrahydropyrazolo[1,5-a]pyridin-5-yl)carbamate

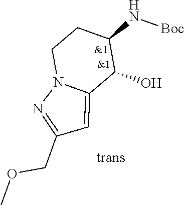 [SMILES: COCc1cc2n(n1)CC[C@@H](NC(=O)OC(C)(C)C)[C@@H]2O]

Step 8. A resealable reaction vial was charged with tert-butyl (2-(methoxymethyl)-4-oxo-4,5,6,7-tetrahydropyrazolo[1,5-a]pyridin-5-yl)carbamate (660 mg, 2.23 mmol) in MeOH (15 mL) and a stir bar, NaBH₄ (127 mg, 3.35 mmol) was added at 0° C., and the reaction was stirred at 0° C. for 1 hour. The reaction mixture was quenched with 10H₂O·Na₂SO₄. The resulting mixture was stirred at 0° C. for 30 min. The reaction mixture was filtered (through a pad of Celite), the pad was washed with DCM, and the filtrate was concentrated in vacuo. This afforded rac-tert-butyl ((4S,5R)-4-hydroxy-2-(methoxymethyl)-4,5,6,7-tetrahydropyrazolo[1,5-a]pyridin-5-yl)carbamate (630 mg, 66%) as a yellow-green amorphous solid. m/z (ES⁺) [M+H]⁺=298.20; HPLC tR=0.492 min.

### rac-(4R,5R)-5-((tert-butoxycarbonyl)amino)-2-(methoxymethyl)-4,5,6,7-tetrahydropyrazolo[1,5-a]pyridin-4-yl 4-nitrobenzoate

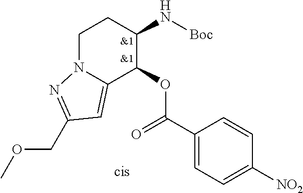 [SMILES: COCc1cc2n(n1)CC[C@@H](NC(=O)OC(C)(C)C)[C@H]2OC(=O)c1ccc([N+](=O)[O-])cc1]

Step 9. To a mixture of rac-tert-butyl ((4S,5R)-4-hydroxy-2-(methoxymethyl)-4,5,6,7-tetrahydropyrazolo[1,5-a]pyridin-5-yl)carbamate (600 mg, 2.02 mmol), 4-nitrobenzoic acid (405 mg, 2.42 mmol) and triphenylphosphane (1.22 g, 4.64 mmol) in THF (15 mL) was added DIAD (938 mg, 4.64 mmol) dropwise at 0° C. under nitrogen atmosphere. The mixture was allowed to slowly warm to r.t., and then stirred for 12 hours. The mixture was diluted with water (30 mL), and the aq. phase was extracted with EtOAc (3×60 mL). The combined organic layers were washed with brine, dried over sodium sulfate, filtered, and concentrated in vacuo. The resulting crude material was purified by flash chromatography (mobile phase: acetonitrile/water; gradient: 20% to 70% in 20 min; wavelength: 254 nm). The desired fractions were combined, and then concentrated in vacuo to afford rac-(4R,5R)-5-((tert-butoxycarbonyl)amino)-2-(methoxymethyl)-4,5,6,7-tetrahydropyrazolo[1,5-a]pyridin-4-yl 4-nitrobenzoate (600 mg, 31%) as a yellow amorphous solid. m/z (ES⁺) [M+H]⁺=447.10; HPLC tR=0.812 min.

### rac-tert-butyl ((4R,5R)-4-hydroxy-2-(methoxymethyl)-4,5,6,7-tetrahydropyrazolo[1,5-a]pyridin-5-yl)carbamate

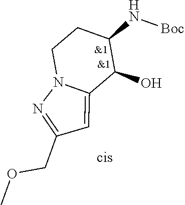 [SMILES: COCc1cc2n(n1)CC[C@@H](NC(=O)OC(C)(C)C)[C@H]2O]

Step 10. A resealable reaction vial was charged with rac-(4R,5R)-5-((tert-butoxycarbonyl)amino)-2-(methoxymethyl)-4,5,6,7-tetrahydropyrazolo[1,5-a]pyridin-4-yl 4-nitrobenzoate (580 mg, 1.30 mmol) and 1M LiOH (3.8 mL, 3.90 mmol) in THE (7 mL)/MeOH (7 mL) and a stir bar, and the resulting mixture was stirred at 20° C. for 1 hour. Then, the pH was adjusted to 9-10 with LiOH (1M), the mixture was diluted with water (15 mL), and the aq. phase was extracted with EtOAc (3×30 mL). The combined organic layers were washed with brine, dried over sodium sulfate, filtered, and then concentrated in vacuo to afford rac-tert-butyl ((4R,5R)-4-hydroxy-2-(methoxymethyl)-4,5,6,7-tetrahydropyrazolo[1,5-a]pyridin-5-yl)carbamate (480 mg, 76%) as a yellow amorphous solid. m/z (ES⁺) [M+H]⁺=298.20; HPLC tR=0.483 min.

### rac-(4R,5R)-5-amino-2-(methoxymethyl)-4,5,6,7-tetrahydropyrazolo[1,5-a]pyridin-4-ol

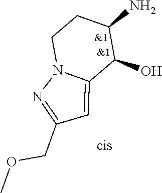

Step 11. A resealable reaction vial was charged with rac-tert-butyl ((4R,5R)-4-hydroxy-2-(methoxymethyl)-4,5,6,7-tetrahydropyrazolo[1,5-a]pyridin-5-yl)carbamate (300 mg, 1.01 mmol) in MeOH (1 mL), followed by 4 M HCl in 1,4-dioxane (1 mL), and a stir bar, and the resulting mixture was stirred at 20° C. for 1 hour. Concentration in vacuo afforded rac-(4R,5R)-5-amino-2-(methoxymethyl)-4,5,6,7-tetrahydropyrazolo[1,5-a]pyridine-4-ol (200 mg, 41%) as a yellow amorphous solid. m/z (ES⁺) [M+H]⁺=198.10; HPLC tR=0.160 min.

### rac-(4R,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol

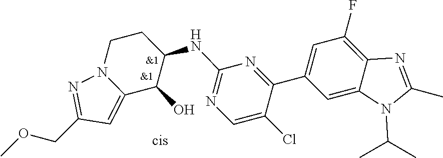

Step 12. A resealable reaction vial was charged with 6-(2-chloro-5-fluoropyrimidin-4-yl)-4-fluoro-1-isopropyl-2-methyl-1H-benzo[d]imidazole (312 mg, 0.92 mmol), rac-(4R,5R)-5-amino-2-(methoxymethyl)-4,5,6,7-tetrahydropyrazolo[1,5-a]pyridin-4-ol (200 mg, 1.01 mmol), DIEA (655 mg, 5.07 mmol), NMP (2 mL) and a stir bar, evacuated and purged with nitrogen three times, and then the resulting mixture was stirred at 150° C. for 1 hour. The resulting crude material was purified by preparative HPLC (column: XBridge Prep OBD C18 Column, 30×150 mm, 5 μm; mobile phase A: water (10 mM NH₄HCO₃)+0.05% NH H₂O; mobile phase B: ACN; flow rate: 60 mL/min; gradient: 19% B to 47% B in 7 min; wavelength: 254 nm & 220 nm; tR1 (min): 7.62). The desired fractions were combined, and then lyophilized to afford rac-(4R,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (90 mg, 18%) as a white solid. m/z (ES⁺) [M+H]⁻=500.10; HPLC tR=1.498 min.

### rel-(4S,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (Compound I-213)

### rel-(4R,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (Compound I-218)

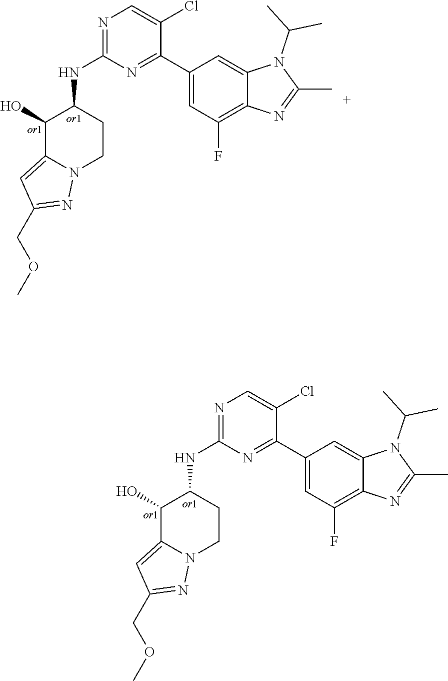 [SMILES: COCc1cc2n(n1)CC[C@H](Nc1ncc(Cl)c(-c3cc(F)c4nc(C)n(C(C)C)c4c3)n1)[C@@H]2O ; COCc1cc2n(n1)CC[C@@H](Nc1ncc(Cl)c(-c3cc(F)c4nc(C)n(C(C)C)c4c3)n1)[C@H]2O]

rac-(4R,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (90 mg, 0.18 mmol) was purified by chiral preparative HPLC (column: CHIRALPAK IC, 2×25 cm, m; mobile phase A: Hex (0.2% TEA); mobile phase B: EtOH/DCM [1:1]HPLC; flow rate: 20 mL/min; gradient: 40% B to 40% B in 15 min; wavelength: 220 nm & 254 nm; tR1 (min): 8.6; tR2 (min): 13.1; sample solvent: EtOH/DCM [1:1]; injection volume: 0.5 mL; number of runs: 3). The desired fractions were combined, and then lyophilized to afford rel-(4S,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (18.3 mg, 41%) as a white solid: m/z (ES⁺) [M+H]⁺=500.10; HPLC tR=0.898 min. ¹H NMR (400 MHz, DMSO-d6) 8.48 (s, 1H), 7.94 (s, 1H), 7.41 (d, J=11.6 Hz, 1H), 7.22 (d, J=7.0 Hz, 1H), 6.17 (s, 1H), 5.62 (d, J=5.8 Hz, 1H), 4.97 (s, 1H), 4.80 (p, J=6.9 Hz, 1H), 4.31-4.16 (m, 4H), 4.05 (dt, J=13.5, 6.8 Hz, 1H), 3.24 (s, 3H), 2.62 (s, 3H), 2.44 (d, J=5.5 Hz, 1H), 2.04 (d, J=13.0 Hz, 1H), 1.57 (d, J=7.1 Hz, 6H) and rel-(4R,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (32.1 mg, 71%) as a white solid: m/z (ES⁺) [M+H]⁺=500.15; HPLC tR=0.898 min. ¹H NMR (400 MHz, DMSO-d6) 8.48 (s, 1H), 7.94 (s, 1H), 7.41 (d, J=11.6 Hz, 1H), 7.23 (d, J=7.0 Hz, 1H), 6.17 (s, 1H), 5.62 (d, J=5.8 Hz, 1H), 4.97 (s, 1H), 4.80 (p, J=6.8 Hz, 1H), 4.30 (s, 2H), 4.26-4.14 (m, 2H), 4.05 (td, J=12.1, 5.1 Hz, 1H), 3.24 (s, 3H), 2.62 (s, 3H), 2.42 (dd, J=11.5, 5.8 Hz, 1H), 2.04 (d, J=13.1 Hz, 1H), 1.56 (dd, J=7.2, 3.4 Hz, 6H).

### Example 15

### (4R,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol and (4S,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (Compounds I-212 and I-214)

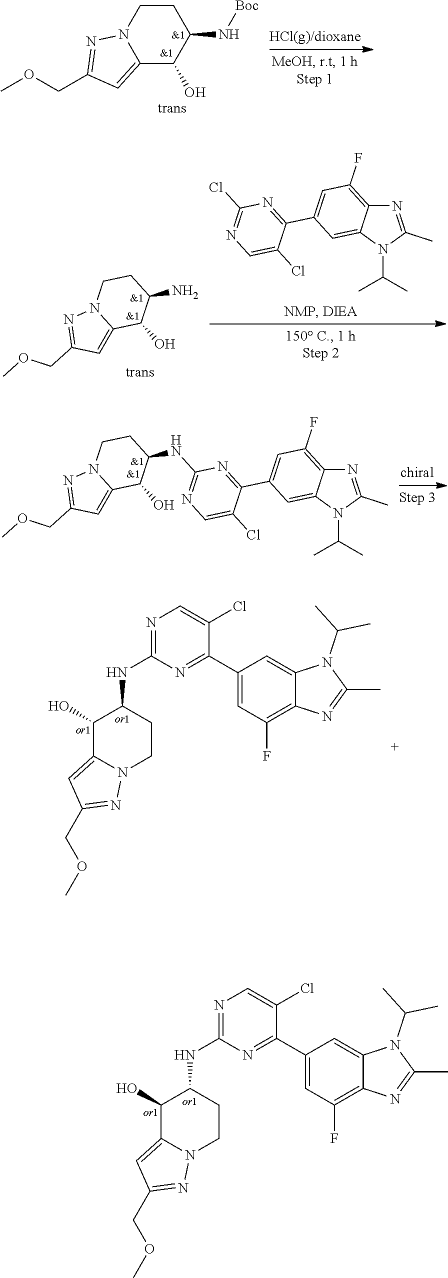

### rac-(4S,5R)-5-amino-2-(methoxymethyl)-4,5,6,7-tetrahydropyrazolo[1,5-a]pyridin-4-ol

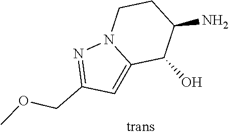

Step 1. A round-bottom flask was charged with rac-tert-butyl ((4S,5R)-4-hydroxy-2-(methoxymethyl)-4,5,6,7-tetrahydropyrazolo[1,5-a]pyridin-5-yl)carbamate (300 mg, 1.01 mmol) in MeOH (1.5 mL), followed by 4 M HCl in 1,4-dioxane (1.5 mL), and a stir bar, and the resulting solution was stirred at 25° C. for 1 hour. Concentration in vacuo afforded crude rac-(4S,5R)-5-amino-2-(methoxymethyl)-4,5,6,7-tetrahydropyrazolo[1,5-a]pyridin-4-ol (320 mg, 161%) as a yellow solid. m/z (ES⁺) [M+H]⁺=198.15; HPLC tR=0.160 min.

### rac-(4R,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol

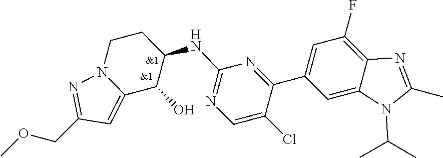

Step 2. A round-bottom flask was charged with rac-(4S,5R)-5-amino-2-(methoxymethyl)-4,5,6,7-tetrahydropyrazolo[1,5-a]pyridin-4-ol (0.32 g, 1.62 mmol), 6-(2,5-dichloropyrimidin-4-yl)-4-fluoro-1-isopropyl-2-methyl-1H-benzo[d]imidazole (0.55 g, 1.62 mmol) and N-ethyl-N-isopropylpropan-2-amine (1.26 g, 9.73 mmol) in NMP (2 mL), and a stir bar, and the resulting solution was stirred for 1 hour at 150° C. The crude product was purified by reverse-phase flash chromatography (column: C18 silica gel; mobile phase, ACN in water; gradient: 20% to 70% in 20 min; wavelength: 254 nm. The desired fractions were combined, and then concentrated in vacuo to afford rac-(4S,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (331 mg, 40%) as a brown solid. m/z (ES⁺) [M+H]⁺=500.15; HPLC tR=0.908 min.

### rel-(4R,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (Compound I-212)

### rel-(4S,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (Compound I-214)

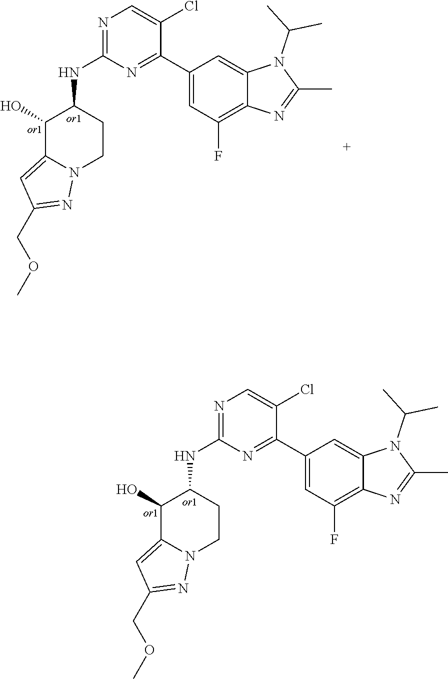 [SMILES: COCc1cc2n(n1)CC[C@H](Nc1ncc(Cl)c(-c3cc(F)c4nc(C)n(C(C)C)c4c3)n1)[C@H]2O ; COCc1cc2n(n1)CC[C@@H](Nc1ncc(Cl)c(-c3cc(F)c4nc(C)n(C(C)C)c4c3)n1)[C@@H]2O]

Step 3. The crude rac-(4R,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (331 mg, 0.66 mmol) from above was purified by chiral preparative HPLC (column: CHIRALPAK IC, 2×25 cm, 5 μm; mobile phase A: Hex (0.2% TEA); mobile phase B: EtOH/DCM [1:1]HPLC; flow rate: 20 mL/min; gradient: 60% B to 60% B in 13 min; wavelength: 220 nm & 254 nm; tR1 (min): 4.53; tR2 (min): 9.75; sample solvent: EtOH/DCM [1:1]; injection volume: 0.7 mL; number of runs: 5). The desired fractions were combined, and then lyophilized to afford rel-(4S,5R)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (74.8 mg, 149 mol, 45.3%) as a white solid: m/z (ES⁺) [M+H]⁺=500.20; HPLC tR=1.430 min ¹H NMR (400 MHz, DMSO-d₆) 8.47 (s, 1H), 7.94 (s, 1H), 7.64 (s, 1H), 7.40 (s, 1H), 6.17 (s, 1H), 5.66 (d, J=6.4 Hz, 1H), 4.79 (dq, J=14.7, 7.0 Hz, 2H), 4.29 (s, 3H), 4.14-3.98 (m, 2H), 3.24 (s, 3H), 2.62 (s, 3H), 2.34 (d, J=9.9 Hz, 1H), 2.04 (s, 1H), 1.57 (d, J=6.9 Hz, 6H) and rel-(4R,5S)-5-((5-chloro-4-(4-fluoro-1-isopropyl-2-methyl-1H-benzo[d]imidazol-6-yl)pyrimidin-2-yl)amino)-2-(methoxymethyl)-4,5,6,7-tetrahydropyrazolo[1,5-a]pyridin-4-ol (86.8 mg, 52.6%) as a white amorphous solid: m/z (ES⁺) [M+H]⁺=500.15; HPLC tR=0.912 min. ¹H NMR (400 MHz, DMSO-d₆) 8.47 (s, 1H), 7.94 (s, 1H), 7.65 (d, J=7.2 Hz, 1H), 7.40 (s, 1H), 6.17 (s, 1H), 5.67 (d, J=6.5 Hz, 1H), 4.79 (dq, J=14.6, 7.0 Hz, 2H), 4.33-4.18 (m, 3H), 4.17-3.99 (m, 2H), 3.24 (s, 3H), 2.62 (s, 3H), 2.37 (d, J=13.4 Hz, 1H), 2.04 (s, 1H), 1.57 (d, J=6.9 Hz, 6H).

### Example 16

### 6′-(5-chloro-2-(((3S,4R)-3-hydroxytetrahydro-2H-pyran-4-yl)amino)pyrimidin-4-yl)spiro[cyclobutane-1,1′-isoindolin]-3′-one (Compound I-223)

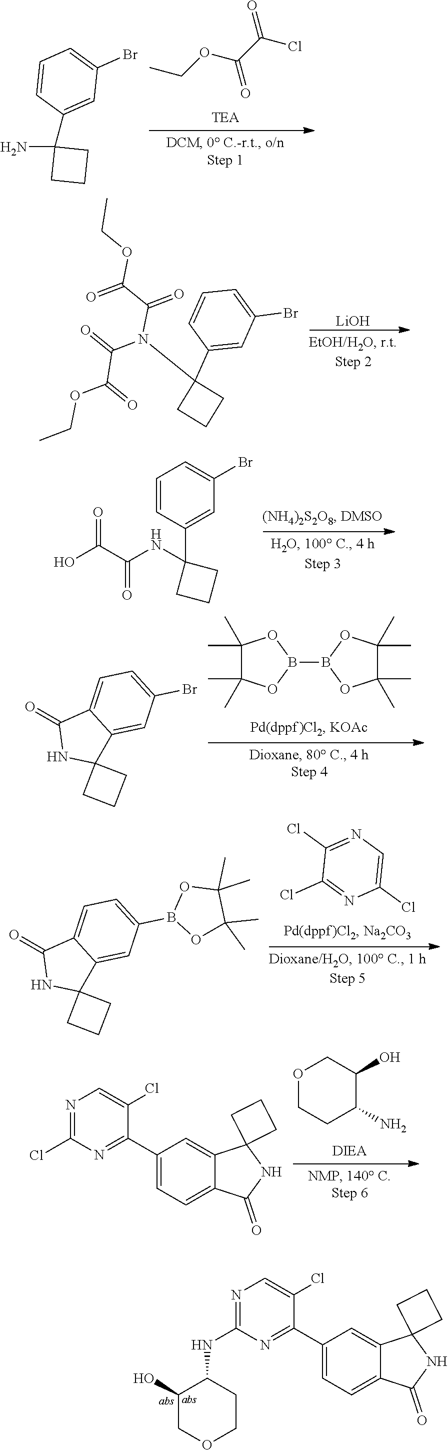

### diethyl 2,2′-((1-(3-bromophenyl)cyclobutyl)azanediyl)bis(2-oxoacetate)

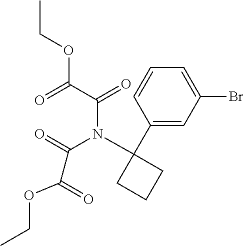 [SMILES: CCOC(=O)C(=O)N(C(=O)C(=O)OCC)C1(c2cccc(Br)c2)CCC1]

Step 1. To a solution of 1-(3-bromophenyl)cyclobutan-1-amine (540 mg, 1 eq., 2.39 mmol) in DCM (4 mL) was added triethylamine (725 mg, 999 μL, 3 eq., 7.16 mmol), and the resulting mixture was stirred at 0° C. for 5 minutes. Next, ethyl 2-chloro-2-oxoacetate (1.63 g, 5 eq., 11.9 mmol) was added dropwise at 0° C., and the reaction was stirred at r.t. overnight. The resulting mixture was concentrated under reduced pressure to afford crude diethyl 2,2′-((1-(3-bromophenyl)cyclobutyl)azanediyl)bis(2-oxoacetate) (750 mg, 1.76 mmol, 73.7%) as a yellow solid. m/z (ES⁺) [M+H+NH₃]⁺=443.05; HPLC tR=0.921 min.

### 2-((1-(3-bromophenyl)cyclobutyl)amino)-2-oxoacetic acid

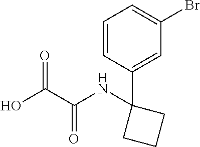 [SMILES: O=C(O)C(=O)NC1(c2cccc(Br)c2)CCC1]

Step 2. To a solution of diethyl 2,2′-((1-(3-bromophenyl)cyclobutyl)azanediyl)bis(2-oxoacetate) (750 mg, 1 eq., 1.76 mmol) in EtOH (8 mL) was added lithium hydroxide (169 mg, 4 eq., 7.04 mmol), and the mixture was stirred at 25° C. for 3 hours. No signs of starting material were found on LC/MS. The pH of the resulting solution was adjusted to 5-6 with aq. HCl, and then the solution was extracted with DCM. The organic layer was dried over sodium sulfate, and then concentrated under reduced pressure to afford crude 2-((1-(3-bromophenyl)cyclobutyl)amino)-2-oxoacetic acid (900 mg, 3.02 mmol, 172%) as a light yellow oil. m/z (ES⁻) [M−H]⁻=295.95, HPLC tR=0.510 min

### 6′-bromospiro[cyclobutane-1,1′-isoindolin]-3′-one

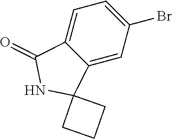 [SMILES: O=C1NC2(CCC2)c2cc(Br)ccc21]

Step 3. A solution of 2-((1-(3-bromophenyl)cyclobutyl)amino)-2-oxoacetic acid (900 mg, 25% Wt, 1 eq., 755 mol), (NH₄)₂S₂O₈ (861 mg, 5 eq., 3.77 mmol) in DMSO (6 mL) and water (0.3 mL) was stirred at 100° C. for 4 hours. The resulting solution was purified using flash chromatography (column: C18; mobile phase A: water; mobile phase B: ACN; flow rate: 60 mL/min; gradient: 30% B to 80% B in 40 min; wavelength: 220 nm & 254 nm), to afford 6′-bromospiro[cyclobutane-1,1′-isoindolin]-3′-one (70 mg, 0.28 mmol, 37%) as a yellow solid. m/z (ES⁺) [M+H]⁺=252.00; HPLC tR=0.701 min. ¹H NMR (400 MHz, DMSO-d6) 9.12 (s, 1H), 8.12 (d, J=1.7 Hz, 1H), 7.66 (dd, J=8.0, 1.6 Hz, 1H), 7.52 (d, J=8.0 Hz, 1H), 2.54 (dd, J=9.2, 2.5 Hz, 2H), 2.43 (ddd, J=13.6, 8.5, 3.9 Hz, 2H), 2.18 (dt, J=11.6, 8.7 Hz, 1H), 1.97 (dq, J=10.2, 4.6 Hz, 1H).

### 6′-(4,4,5,5-tetramethyl-1,3,2-dioxaborolan-2-yl)spiro[cyclobutane-1,1′-isoindolin]-3′-one

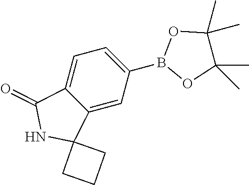 [SMILES: CC1(C)OB(c2ccc3c(c2)C2(CCC2)NC3=O)OC1(C)C]

Step 4. To a stirring solution of 6′-bromospiro[cyclobutane-1,1′-isoindolin]-3′-one (65 mg, 1 eq., 0.26 mmol), bis(pinacolato)diboron (98 mg, 1.5 eq., 0.39 mmol) and potassium acetate (76 mg, 3 eq., 0.77 mmol) in 1,4-dioxane (1 mL) was added Pd(dppf)Cl₂ (32 mg, 0.15 eq., 39 μmol), and the resulting solution was stirred at 80° C. under nitrogen atmosphere for 4 hours. The resulting mixture was filtered, and the filter cake was washed with 1,4-dioxane. The filtrate was concentrated under reduced pressure to afford crude 6′-(4,4,5,5-tetramethyl-1,3,2-dioxaborolan-2-yl)spiro[cyclobutane-1,1′-isoindolin]-3′-one (90 mg, 0.30 mmol, 120%) as a black solid. m/z (ES⁺) [M+H]⁺=300.15; HPLC tR=0.781 min.

### 6′-(2,5-dichloropyrimidin-4-yl)spiro[cyclobutane-1,1′-isoindolin]-3′-one

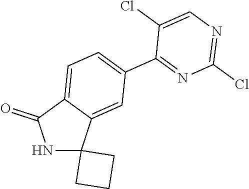 [SMILES: O=C1NC2(CCC2)c2cc(-c3nc(Cl)ncc3Cl)ccc21]

Step 5. To a stirring solution of crude 6′-(4,4,5,5-tetramethyl-1,3,2-dioxaborolan-2-yl)spiro[cyclobutane-1,1′-isoindolin]-3′-one (80 mg, 1 eq., 0.27 mmol), 2,4,5-trichloropyrimidine (74 mg, 1.5 eq., 0.40 mmol) and Na₂CO₃ (85 mg, 3 eq., 0.80 mmol) in 1,4-dioxane (4 mL) and water (0.8 mL) was added [1,1′-bis(diphenylphosphino)ferrocene]dichloropalladium(II) complex with dichloromethane (33 mg, 0.15 eq., 40 mol), and the resulting solution was stirred at 100° C. under nitrogen atmosphere for 1 hour. Next, the mixture was concentrated under reduced pressure, and the resulting residue was dissolved in DMF. The resulting solution was purified using flash chromatography (column: C18, mobile phase A: water; mobile phase B: ACN; flow rate: 60 mL/min; gradient: 20% B to 65% B in 40 min; wavelength: 220 nm & 254 nm). The desired fractions were combined, and then lyophilized to afford 6′-(2,5-dichloropyrimidin-4-yl)spiro[cyclobutane-1,1′-isoindolin]-3′-one (14 mg, 40 μmol, 15%, 92% purity) as a yellow solid. m/z (ES⁺) [M+H]⁻=320.10; HPLC tR=1.168 min.

### 6′-(5-chloro-2-(((3R,4S)-3-hydroxytetrahydro-2H-pyran-4-yl)amino)pyrimidin-4-yl)spiro[cyclobutane-1,1′-isoindolin]-3′-one

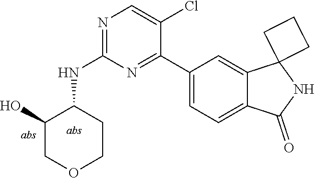 [SMILES: O=C1NC2(CCC2)c2cc(-c3nc(N[C@@H]4CCOC[C@H]4O)ncc3Cl)ccc21]

Step 6. A solution of 6′-(2,5-dichloropyrimidin-4-yl)spiro[cyclobutane-1,1′-isoindolin]-3′-one (12 mg, 1 eq., 37 mol), (3S,4R)-4-aminotetrahydro-2H-pyran-3-ol hydrochloride (4.9 mg, 1.1 eq., 42 μmol) and diisopropylethylamine (19 mg, 26 μL, 4 eq., 0.15 mmol) in NMP (0.5 mL) was stirred at 140° C. for 30 minutes, with monitoring by LC/MS. The resulting solution was purified by prep-HPLC (column: XBridge Shield RP18 OBD Column, 30×150 mm, 5 μm; mobile phase A: water (10 mM NH₄HCO₃+0.05% NH₃H₂O); mobile phase B: ACN; flow rate: 60 mL/min; gradient: 15% B to 40% B in 7 min; wavelength: 220 nm & 254 nm; tR1 (min): 6.97; number of runs: 1) to afford 6′-(5-chloro-2-(((3R,4S)-3-hydroxytetrahydro-2H-pyran-4-yl)amino)pyrimidin-4-yl)spiro[cyclobutane-1,1′-isoindolin]-3′-one (3.8 mg, 9.4 mol, 25%, 99.4% purity) as a light-pink amorphous solid. m/z (ES⁺) [M+H]⁺=401.05; HPLC tR=0.822 min. ¹H NMR (400 MHz, DMSO-d6) 9.17 (s, 1H), 8.45 (s, 1H), 8.16 (s, 1H), 7.80 (s, 1H), 7.71 (d, J=7.8 Hz, 1H), 7.55 (s, 1H), 4.95 (d, J=5.3 Hz, 1H), 3.81 (dt, J=10.9, 5.3 Hz, 3H), 3.48 (s, 1H), 3.31 (d, J=11.0 Hz, 1H), 3.04 (t, J=10.4 Hz, 1H), 2.70-2.53 (m, 2H), 2.45 (td, J=8.2, 4.0 Hz, 2H), 2.13 (dt, J=11.1, 8.4 Hz, 1H), 2.08-1.85 (m, 2H), 1.51 (tt, J=13.9, 7.1 Hz, 1H).

### Example 17

### (S)-5-(3-(dimethylamino)pyrrolidin-1-yl)pyridin-2-amine (Synthetic Intermediate)

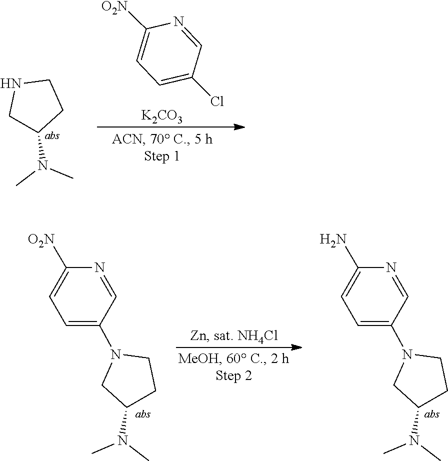

### (S)—N,N-dimethyl-1-(6-nitropyridin-3-yl)pyrrolidin-3-amine

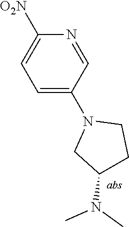 [SMILES: CN(C)[C@H]1CCN(c2ccc([N+](=O)[O-])nc2)C1]

Step 1. A round-bottom flask was charged with (S)—N,N-dimethylpyrrolidin-3-amine (500 mg, 4.38 mmol), 5-chloro-2-nitropyridine (1.04 g, 6.57 mmol), K₂CO₃ (2.42 g, 17.50 mmol) in ACN (10 mL) and a stir bar, and the resulting solution was stirred for 5 hours at 70° C. Then, the reaction mixture was concentrated in vacuo. The residue was purified by reverse-phase flash chromatography (column: C18 silica gel; mobile phase: ACN in water; gradient: 10% to 70% in 15 min; wavelength: 254 nm). The desired fractions were combined, and then lyophilized to afford (S)—N,N-dimethyl-1-(6-nitropyridin-3-yl)pyrrolidin-3-amine (600 mg, 58.0%) as a yellow oil. m/z (ES⁺) [M+H]⁺=237.15; HPLC tR=0.417 min.

### (S)-5-(3-(dimethylamino)pyrrolidin-1-yl)pyridin-2-amine

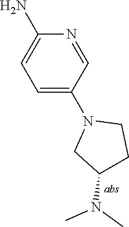

Step 2. A resealable reaction vial was charged with (S)—N,N-dimethyl-1-(6-nitropyridin-3-yl)pyrrolidin-3-amine (400 mg, 1.69 mmol), zinc (443 mg, 6.77 mmol), sat'd. NH₄Cl (2 mL), MeOH (6 mL) and a stir bar, before being evacuated and purged with nitrogen three times. The resulting mixture was stirred at 60° C. for 2 hours. Next, the reaction mixture was filtered, the solid was washed with MeOH, and the filtrate was concentrated in vacuo. The residue was purified by reverse-phase flash chromatography (column: C18 silica gel; mobile phase: ACN in water; gradient: 10% to 80% in 20 min; wavelength: 254 nm). Concentration in vacuo afforded (S)-5-(3-(dimethylamino)pyrrolidin-1-yl)pyridin-2-amine (200 mg, 57.3%) as a pink amorphous solid. m/z (ES⁺) [M+H]⁺=207.00; HPLC tR=0.495

### Example 18

### tert-butyl (5-(2,5-dichloropyrimidin-4-yl)-4-methylthiazol-2-yl)(methyl)carbamate (Synthetic Intermediate)

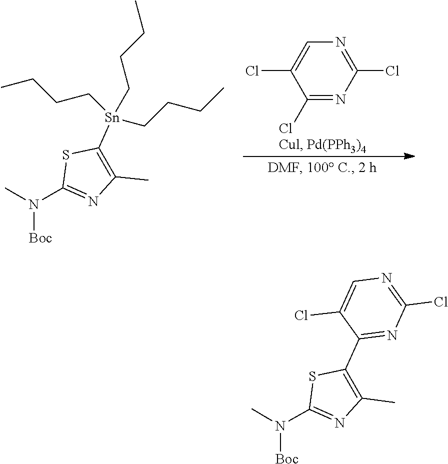

To a solution of tert-butyl methyl(4-methyl-5-(tributylstannyl)thiazol-2-yl) carbamate (500 mg, 1 eq., 966 μmol) in DMF (8 mL) were added 2,4,5-trichloropyrimidine (266 mg, 1.5 eq., 1.45 mmol) and copper(I) iodide (18.4 mg, 0.1 eq., 96.6 μmol). Next, nitrogen was bubbled through the reaction mixture for 1 minute, and then Pd(Ph₃P)₄ (112 mg, 0.1 eq., 96.6 μmol) was added. The resulting reaction mixture was heated at 100° C. with vigorous stirring for 2 hours. The reaction mixture was cooled, and then treated with water (20 mL). The organic layer was extracted with EtOAc, dried over sodium sulfate, and then concentrated in vacuo. The crude residue was purified by flash chromatography (mobile phase A: water; mobile phase B: ACN; flow rate: 60 mL/min; gradient: 30% B to 60% B in 9 min). The desired fractions were combined, and then concentrated in vacuo to afford the title compound, tert-butyl (5-(2,5-dichloropyrimidin-4-yl)-4-methylthiazol-2-yl)(methyl)carbamate (216 mg, 576 μmol, 59.6%), as a light-yellow solid. m/z (ES⁺) [M+H]⁺=375.05; HPLC tR=1.086 min.

### Example 19

### rac-(1R,3S)-3-(2-chloro-5-fluoropyrimidin-4-yl)cyclopentyl isopropylcarbamate (Synthetic Intermediate)

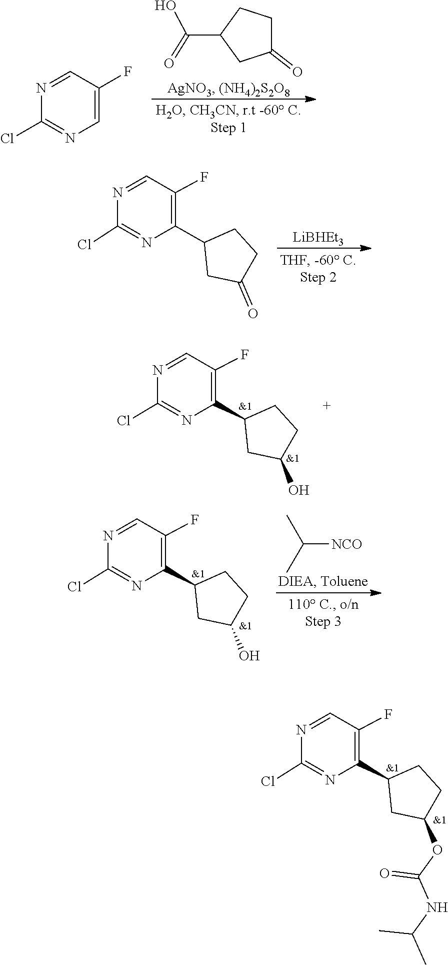

### 3-(2-chloro-5-fluoropyrimidin-4-yl)cyclopentan-1-one

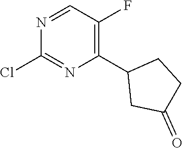 [SMILES: O=C1CCC(c2nc(Cl)ncc2F)C1]

Step 1. To a solution of 2-chloro-5-fluoropyrimidine (1.0 g, 1 eq., 7.5 mmol), 3-oxocyclopentane-1-carboxylic acid (2.9 g, 3 eq., 23 mmol) and (nitrooxy)silver (5.1 g, 4 eq., 30 mmol) in ACN (10 mL) and H₂O (10 mL) was added (NH₄)₂S₂O₈ (8.6 g, 5 eq., 38 mmol), and the resulting mixture was stirred at 60° C. for 2 hours. Next, the reaction mixture was quenched with concentrated aq. ammonium hydroxide (0.8 mL) and water (3.2 mL), diluted with brine, and filtered through Celite. The filtrate was then extracted with DCM (3×50 mL) and the organic extracts were dried over sodium sulfate, filtered, and then concentrated in vacuo. The residue was purified by flash chromatography (mobile phase A: heptane; mobile phase B: EtOAc; gradient: 0 to 50% B) to afford 3-(2-chloro-5-fluoropyrimidin-4-yl)cyclopentan-1-one (660 mg, 2.63 mmol, 35%, 85.6% purity) as a yellow oil. m/z (ES⁺) [M+H]⁺=215.05; HPLC tR=0.651 min.

### rac-(1R,3S)-3-(2-chloro-5-fluoropyrimidin-4-yl)cyclopentan-1-ol

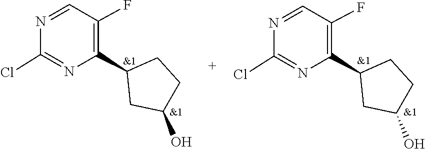 [SMILES: O[C@@H]1CC[C@H](c2nc(Cl)ncc2F)C1 ; O[C@H]1CC[C@H](c2nc(Cl)ncc2F)C1]

Step 2. A solution of 3-(2-chloro-5-fluoropyrimidin-4-yl) cyclopentan-1-one (750 mg, 1 eq., 3.49 mmol) in THE (20 mL) was degassed under vacuum and purged with dry nitrogen, and then cooled to −60° C. To this solution was added dropwise a 1.0 M solution of LiBHEt₃ (648 mg, 1.75 eq., 6.12 mmol) in THF, at a rate which maintained the internal temperature below −55° C. Next, then reaction mixture was stirred at −60° C. for 1.5 hours. Then, the mixture was quenched with sat'd. aq. NaHCO₃ (40 mL) in the temperature range of −40° C. to −30° C. To the mixture was then added dropwise hydrogen peroxide (30% aq., 1.6 g), the internal temperature was maintained between −10° C. and 0° C., and the mixture was stirred at 10° C. for 1 hour. Next, the mixture was extracted with EtOAc (3×50 mL), and the combined organic layers were washed with sat'd. aq. Na₂SO₃ (2×200 mL) and sat'd. aq. NaCl (2×200 mL), dried over anhydrous Na₂SO₄, and then concentrated in vacuo. The resulting residue was purified by silica gel column chromatography, eluting with DCM/MeOH (20:1), to afford crude product (250 mg, 75%). This crude product was purified by preparative HPLC to afford rac-(1R,3S)-3-(2-chloro-5-fluoropyrimidin-4-yl)cyclopentan-1-ol (160 mg, 739 μmol, 21.1%) as a yellow solid. m/z (ES⁺) [M+H]⁺=217.00; HPLC tR=0.629 min.

### rac-(1R,3S)-3-(2-chloro-5-fluoropyrimidin-4-yl)cyclopentyl isopropylcarbamate

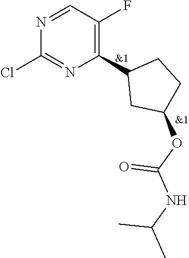

Step 3. To a solution of rac-(1R,3S)-3-(2-chloro-5-fluoropyrimidin-4-yl)cyclopentan-1-ol (140 mg, 1 eq., 646 μmol) and 2-isocyanatopropane (82.5 mg, 1.5 eq., 969 mol) in toluene (6 mL) was added DIEA (251 mg, 3 eq., 1.94 mmol), and the resulting solution was stirred at 110° C. overnight. Next, the mixture was diluted with EtOAc (10 mL) and washed with brine (3×20 mL), and the combined organic phases were dried over anhydrous Na₂SO₄, and then concentrated in vacuo to afford rac-(1R,3S)-3-(2-chloro-5-fluoropyrimidin-4-yl)cyclopentyl isopropylcarbamate (110 mg, 0.32 mmol, 50%, 89% purity). This crude product was used directly as an intermediate in further chemistry without any further purification. m/z (ES⁺) [M+H]⁺=302.05; HPLC tR=1.078 min.

### Example 20

### 6-(2,5-dichloropyrimidin-4-yl)-4-isopropylquinazoline (Synthetic Intermediate)

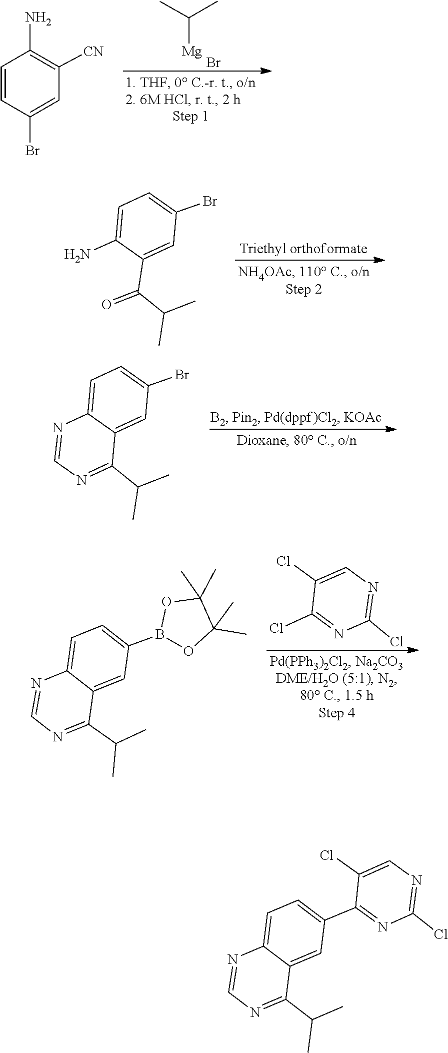

### 1-(2-amino-5-bromophenyl)-2-methylpropan-1-one

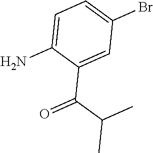

Step 1. To a solution of 2-amino-5-bromobenzonitrile (5 g, 0.03 mol) in THF (100 mL) was added dropwise 1M isopropylmagnesium bromide in THF (127 mL, 0.127 mol) at 0° C. under N₂ atmosphere. The reaction mixture was stirred at 0° C. for 30 min, then warmed to 25° C. and allowed to react for 16 hours. Next, a solution of aq. HCl (6 M, 25 mL) was added dropwise, and the reaction mixture was stirred for another 30 minutes until its pH was between 6 and 7. The reaction was quenched with water (20 mL) and extracted with EtOAc (3×40 mL), and the combined organic extracts were washed with brine (30 mL), dried over anhydrous Na₂SO₄, and then concentrated in vacuo. The residue was purified by flash chromatography (column: C18 silica gel; mobile phase: ACN in water; gradient: 10% to 50% in 10 min; wavelength: 220 nm) to afford 1-(2-amino-5-bromophenyl)-2-methylpropan-1-one (5 g, 0.02 mol, 80%, 95% purity) as a yellow solid. m/z (ES⁺) [M+H]⁺=241.85; HPLC tR=1.046 min.

### 6-bromo-4-isopropylquinazoline

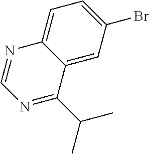

Step 2. A round-bottom flask was charged with 1-(2-amino-5-bromophenyl)-2-methylpropan-1-one (5 g, 0.02 mol), triethoxymethane (9 g, 0.06 mol), ammonium acetate (5 g, 0.06 mol) and a stir bar, and the resulting solution was stirred at 110° C. for 12 hours. The resulting mixture was concentrated in vacuo, and the crude residue was purified by silica gel column chromatography (100 g), eluting with PE/EtOAc (20:1) to afford 6-bromo-4-isopropylquinazoline (4.4 g, 17 mmol, 80%, 95% purity) as a light-yellow oil. m/z (ES⁺) [M+H]=250.85; HPLC tR=0.996 min.

### 4-isopropyl-6-(4,4,5,5-tetramethyl-1,3,2-dioxaborolan-2-yl)quinazoline

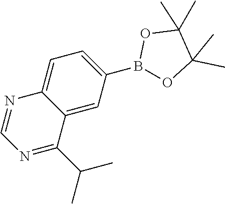 [SMILES: CC(C)c1ncnc2ccc(B3OC(C)(C)C(C)(C)O3)cc12]

Step 3. To a solution of 6-bromo-4-isopropylquinazoline (1 g, 4 mmol) and 4,4,4′,4′,5,5,5′,5′-octamethyl-2,2′-bi(1,3,2-dioxaborolane) (2 g, 6 mmol) in 1,4-dioxane (20 mL), were added PdCl₂(dppf)-CH₂Cl₂ adduct (0.3 g, 0.4 mmol) and potassium acetate (1 g, 0.01 mol), and the resulting mixture was stirred at 80° C. under nitrogen atmosphere for 16 hours. Next, the reaction was quenched with water (10 mL) and extracted with EtOAc (3×15 mL), and the combined organic extracts were washed with brine (20 mL), dried over anhydrous Na₂SO₄, and then concentrated in vacuo. The crude residue was purified by flash chromatography (column: C18 silica gel; mobile phase: ACN in water; gradient: 20% to 60% in 10 min; wavelength: 254 nm) to afford 4-isopropyl-6-(4,4,5,5-tetramethyl-1,3,2-dioxaborolan-2-yl)quinazoline (875 mg, 2.8 mmol, 70%, 95% purity) as an off-white solid. m/z (ES⁺) [M+H]⁺=299.10; HPLC tR=1.298 min.

### 6-(2,5-dichloropyrimidin-4-yl)-4-isopropylquinazoline

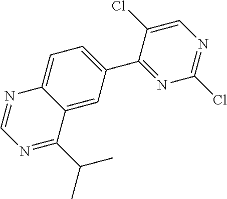 [SMILES: CC(C)c1ncnc2ccc(-c3nc(Cl)ncc3Cl)cc12]

Step 4. To a solution of 4-isopropyl-6-(4,4,5,5-tetramethyl-1,3,2-dioxaborolan-2-yl)quinazoline (375 mg, 1.26 mmol) and 2,4,5-trichloropyrimidine (277 mg, 1.51 mmol) in DME (2.5 mL) and H₂O (0.50 mL) were added sodium carbonate (400 mg, 3.77 mmol) and bis-(triphenylphosphino)-palladium chloride (88.3 mg, 126 μmol), and the resulting mixture was stirred at 80° C. under nitrogen atmosphere for 1.5 hours. The reaction was quenched with water (2 mL) and extracted with EtOAc (3×3 mL), and the combined organic extracts were washed with brine (10 mL), dried over anhydrous Na₂SO₄, and then concentrated in vacuo. The residue was purified by flash chromatography (column: C18 silica gel; mobile phase: ACN in water; gradient: 60% to 70% in 5 min; wavelength: 220 nm) to afford 6-(2,5-dichloropyrimidin-4-yl)-4-isopropylquinazoline (270 mg, 846 μmol, 67.3%, 100% purity) as a yellow solid. m/z (ES⁺) [M+H]⁺=318.95; HPLC tR=1.224 min.

### Example 21

### (S)-3-(5-chloro-2-(((3S,4R)-3-hydroxytetrahydro-2H-pyran-4-yl)amino)pyrimidin-4-yl)-5,6,7,8-tetrahydro-4H-pyrazolo[1,5-a]azepin-4-ol and (R)-3-(5-chloro-2-(((3S,4R)-3-hydroxytetrahydro-2H-pyran-4-yl)amino)pyrimidin-4-yl)-5,6,7,8-tetrahydro-4H-pyrazolo[1,5-a]azepin-4-ol (Compounds I-86 & I-87)

### ethyl 1-(5-methoxy-5-oxopentyl)-1H-pyrazole-5-carboxylate

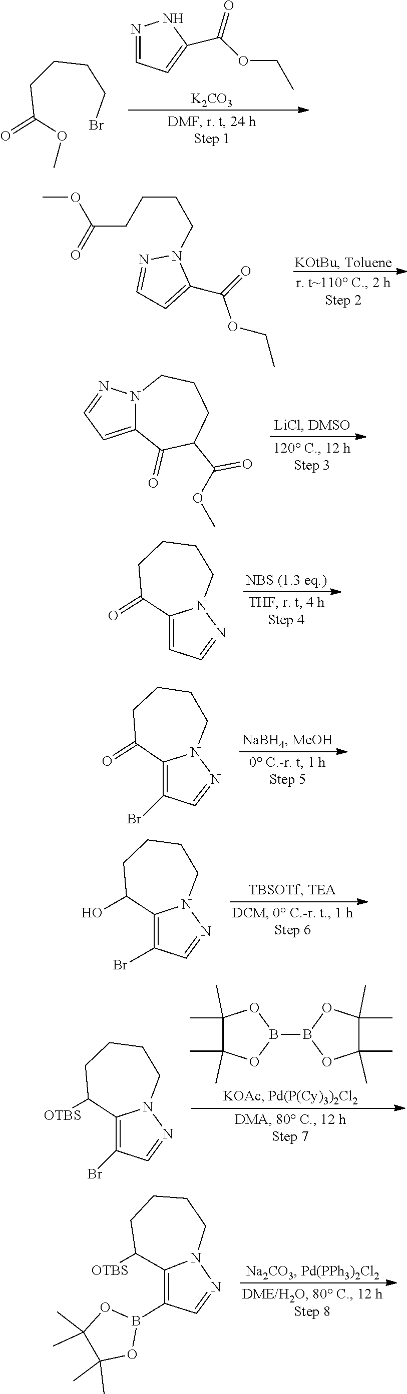

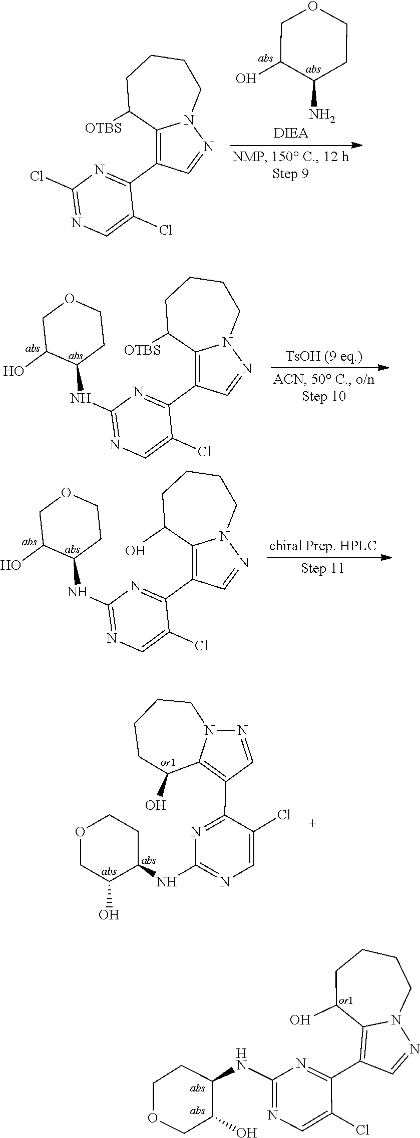

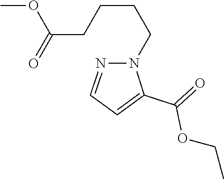 [SMILES: CCOC(=O)c1ccnn1CCCCC(=O)OC]

Step 1. To a mixture of ethyl 1H-pyrazole-5-carboxylate (10 g, 71 mmol) and K₂CO₃ (15 g, 0.11 mol) in DMF (100 mL) was added methyl 5-bromopentanoate (17 g, 86 mmol) in portions at 25° C. under nitrogen atmosphere, and the resulting mixture was stirred at 25° C. for 24 hours. Next, the mixture was diluted with H₂O (150 mL), and the aq. phase was extracted with EtOAc (3×300 mL). The combined organic layers were washed with brine, dried over sodium sulfate, filtered, and concentrated in vacuo. The residue was purified by silica gel chromatography, eluting with PE/EtOAc (8:1). The desired fractions were combined, and then concentrated in vacuo to afford crude ethyl 1-(5-methoxy-5-oxopentyl)-1H-pyrazole-5-carboxylate (11.2 g, 44.0 mmol) as a yellow oil. LC/MS: RT=0.767 min, m/z [M+H]⁺=255.15.

### 4-oxo-5,6,7,8-tetrahydro-4H-pyrazolo[1,5-a]azepine-5-carboxylate

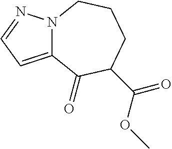

Step 2. A mixture of ethyl 1-(5-methoxy-5-oxopentyl)-1H-pyrazole-5-carboxylate (11.2 g, 44.0 mmol) and potassium 2-methylpropan-2-olate (7.94 g, 70.7 mmol) in toluene (130 mL) was stirred at 25° C. for 10 min, and then at 110° C. for 1.5 hours. The mixture was quenched with sat'd. aq. NH₄Cl, the pH was adjusted to 7 with 1 M HCl, and the aq. phase was extracted with EtOAc (3×300 mL). The combined organic layers were washed with brine, dried over sodium sulfate, filtered, and then concentrated in vacuo to afford methyl 4-oxo-5,6,7,8-tetrahydro-4H-pyrazolo[1,5-a]azepine-5-carboxylate (11 g, 53 mmol, 120%) as a yellow oil LC/MS: RT=1.008 min, m/z [M+H]⁺=209.05.

### 5,6,7,8-tetrahydro-4H-pyrazolo[1,5-a]azepin-4-one

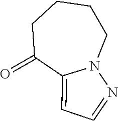 [SMILES: O=C1CCCCn2nccc21]

Step 3. A round-bottom flask was charged with a solution of methyl 4-oxo-5,6,7,8-tetrahydro-4H-pyrazolo[1,5-a]azepine-5-carboxylate (11 g, 53 mmol) and lithium chloride (3.4 g, 79 mmol) in DMSO (130 mL), and a stir-bar, and the solution was stirred at 120° C. for 12 hours. Next, the reaction mixture was diluted with H₂O (70 mL), and the aq. phase was extracted with EtOAc (3×150 mL) three times. The combined organic layers were washed with brine, dried over sodium sulfate, filtered, and then concentrated in vacuo. The resulting residue was purified by reverse-phase flash chromatography (column: C18 silica gel; mobile phase: ACN in water; gradient: 0% to 50% in 25 min; wavelength: 254 nm). The desired fractions were combined, and then concentrated in vacuo to afford 5,6,7,8-tetrahydro-4H-pyrazolo[1,5-a]azepin-4-one (1.8 g, 9.6 mmol, 18%, 80% purity) as a brown oil. LC/MS: RT=0.693 min, m/z [M+H]⁺=151.15.

### 3-bromo-5,6,7,8-tetrahydro-4H-pyrazolo[1,5-a]azepin-4-one

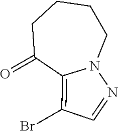 [SMILES: O=C1CCCCn2ncc(Br)c21]

Step 4. To a mixture of 5,6,7,8-tetrahydro-4H-pyrazolo[1,5-a]azepin-4-one (1.8 g, 12 mmol) in THE (20 mL) was added NBS (2.8 g, 16 mmol) in portions at 0° C. under nitrogen atmosphere, and the resulting mixture was stirred at 25° C. for 4 hours. Next, the mixture was diluted with H₂O (50 mL), and the aq. phase was extracted with EtOAc (3×100 mL). The combined organic layers were washed with brine, dried over sodium sulfate, filtered, and then concentrated in vacuo. The residue was purified by silica gel chromatography, eluting with PE/EtOAc (8:1). The desired fractions were combined, and then concentrated in vacuo to afford 3-bromo-5,6,7,8-tetrahydro-4H-pyrazolo[1,5-a]azepin-4-one (2.7 g, 12 mmol, 98%) as a brown oil. LC/MS: RT=0.893 min, m/z [M+H]⁺=229.00.

### 3-bromo-5,6,7,8-tetrahydro-4H-pyrazolo[1,5-a]azepin-4-ol

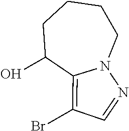 [SMILES: OC1CCCCn2ncc(Br)c21]

Step 5. To a mixture of 3-bromo-5,6,7,8-tetrahydro-4H-pyrazolo[1,5-a]azepin-4-one (2.7 g, 12 mmol) in MeOH (30 mL) was added NaBH₄ (0.49 g, 13 mmol) in portions at 0° C. under nitrogen atmosphere, and the resulting mixture was stirred at 25° C. for 1 hour. Next, sodium sulfate decahydrate was added, and the mixture was stirred for 120 minutes. The solid was filtered out, washed with EtOAc, and then the combined filtrates were concentrated in vacuo to afford 3-bromo-5,6,7,8-tetrahydro-4H-pyrazolo[1,5-a]azepin-4-ol (2.5 g, 12.0 mmol, 92%) as a colorless oil. LC/MS: RT=0.852 min, m/z [M+H]⁺=231.00.

### 3-bromo-4-((tert-butyldimethylsilyl)oxy)-5,6,7,8-tetrahydro-4H-pyrazolo[1,5-a]azepine

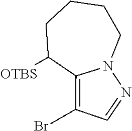 [SMILES: CC(C)(C)[Si](C)(C)OC1CCCCn2ncc(Br)c21]

Step 6. To a mixture of 3-bromo-5,6,7,8-tetrahydro-4H-pyrazolo[1,5-a]azepin-4-ol (1.25 g, 5.41 mmol) and TEA (1.64 g, 16.2 mmol) in DCM (15 mL) was added trifluoromethanesulfonic acid tert-butyldimethylsilyl ester (4.29 g, 16.2 mmol) dropwise at 0° C. under nitrogen atmosphere, and the resulting mixture was stirred at 25° C. for 1 hour. Next, the mixture was quenched with sat'd. aq. NaHCO₃ and diluted with H₂O (50 mL). The aq. phase was extracted with EtOAc (3×100 mL), and the combined organic layers were washed with brine, dried over sodium sulfate, filtered, and then concentrated in vacuo. The residue was purified by reverse-phase flash chromatography (column: C18 silica gel; mobile phase: ACN in water; gradient: 0% to 100% in 25 min; wavelength: 254 nm). The desired fractions were combined, and then concentrated in vacuo to afford 3-bromo-4-((tert-butyldimethylsilyl)oxy)-5,6,7,8-tetrahydro-4H-pyrazolo[1,5-a]azepine (1.28 g, 3.0 mmol, 55%, 80% purity) as a colorless oil. LC/MS: RT=1.368 min, m/z [M+H]⁺=345.10.

### 4-((tert-butyldimethylsilyl)oxy)-3-(4,4,5,5-tetramethyl-1,3,2-dioxaborolan-2-yl)-5,6,7,8-tetrahydro-4H-pyrazolo[1,5-a]azepine

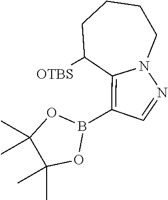 [SMILES: CC1(C)OB(c2cnn3c2C(O[Si](C)(C)C(C)(C)C)CCCC3)OC1(C)C]

Step 7. To a solution of 3-bromo-4-((tert-butyldimethylsilyl)oxy)-5,6,7,8-tetrahydro-4H-pyrazolo[1,5-a]azepine (1 g, 3 mmol) and 4,4,4′,4′,5,5,5′,5′-octamethyl-2,2′-bi(1,3,2-dioxaborolane) (1 g, 6 mmol) in DMA (10 mL) were added potassium acetate (0.9 g, 9 mmol) and dichloro(tricyclohexylphosphine)palladium (II) (0.2 g, 0.3 mmol), and the resulting mixture was stirred at 80° C. under a nitrogen atmosphere for 12 hours. Next, the reaction mixture was quenched with sat'd. aq. NaHCO₃ and diluted with H₂O (50 mL). The aq. phase was extracted with EtOAc (3×100 mL) three times, and the combined organic layers were washed with brine, dried over sodium sulfate, filtered, and then concentrated in vacuo. The residue was purified by reverse-phase flash chromatography (column: C18 silica gel; mobile phase: ACN in water; gradient: 0% to 100% in 25 min; wavelength: 254 nm). The desired fractions were combined, and then concentrated in vacuo to afford 4-((tert-butyldimethylsilyl)oxy)-3-(4,4,5,5-tetramethyl-1,3,2-dioxaborolan-2-yl)-5,6,7,8-tetrahydro-4H-pyrazolo[1,5-a]azepine (640 mg, 1.5 mmol, 50%, 94% purity) as a colorless oil. LC/MS: RT=1.867 min, m/z [M+H]⁺=393.20.

### 4-((tert-butyldimethylsilyl)oxy)-3-(2,5-dichloropyrimidin-4-yl)-5,6,7,8-tetrahydro-4H-pyrazolo[1,5-a]azepine

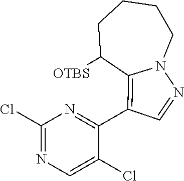 [SMILES: CC(C)(C)[Si](C)(C)OC1CCCCn2ncc(-c3nc(Cl)ncc3Cl)c21]

Step 8. To a solution of 4-((tert-butyldimethylsilyl)oxy)-3-(4,4,5,5-tetramethyl-1,3,2-dioxaborolan-2-yl)-5,6,7,8-tetrahydro-4H-pyrazolo[1,5-a]azepine (550 mg, 1.40 mmol) and 2,4,5-trichloropyrimidine (386 mg, 2.10 mmol) in DME (5 mL) and H₂O (1 mL), were added Na₂CO₃ (446 mg, 4.20 mmol) and bis-(triphenylphosphino)-palladium chloride (98.4 mg, 0.14 mmol), and the resulting mixture was stirred at 80° C. under a nitrogen atmosphere for 12 hours. The crude product was concentrated in vacuo, and the resulting residue was purified by reverse-phase flash chromatography (column: C18 silica gel; mobile phase: ACN in water; gradient: 0% to 100% in 25 min; wavelength: 254 nm). The desired fractions were combined, and then concentrated in vacuo to afford 4-((tert-butyldimethylsilyl)oxy)-3-(2,5-dichloropyrimidin-4-yl)-5,6,7,8-tetrahydro-4H-pyrazolo[1,5-a]azepine (310 mg, 0.60 mmol, 43%, 80% purity) as a brown solid. LC/MS: RT=1.393 min, m/z [M+H]⁺=413.15.

### (3S,4R)-4-((4-(4-((tert-butyldimethylsilyl)oxy)-5,6,7,8-tetrahydro-4H-pyrazolo[1,5-a]azepin-3-yl)-5-chloropyrimidin-2-yl)amino)tetrahydro-2H-pyran-3-ol

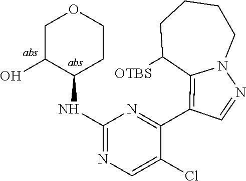 [SMILES: CC(C)(C)[Si](C)(C)OC1CCCCn2ncc(-c3nc(N[C@@H]4CCOCC4O)ncc3Cl)c21]

Step 9. A round-bottom flask was charged with a solution of 4-((tert-butyldimethylsilyl)oxy)-3-(2,5-dichloropyrimidin-4-yl)-5,6,7,8-tetrahydro-4H-pyrazolo[1,5-a]azepine (300 mg, 0.73 mmol), (3S,4R)-4-aminotetrahydro-2H-pyran-3-ol hydrochloride (123 mg, 0.80 mmol) and DIEA (281 mg, 2.18 mmol) in NMP (3 mL), and a stir bar, and the solution was stirred at 150° C. for 12 hours. The crude product was concentrated in vacuo, and the resulting residue was purified by reverse-phase flash chromatography (column: C18 silica gel; mobile phase: ACN in water; gradient: 0% to 100% in 20 min; wavelength: 254 nm). The desired fractions were combined, and then concentrated in vacuo to afford (3S,4R)-4-((4-(4-((tert-butyldimethylsilyl)oxy)-5,6,7,8-tetrahydro-4H-pyrazolo[1,5-a]azepin-3-yl)-5-chloropyrimidin-2-yl)amino)tetrahydro-2H-pyran-3-ol (260 mg, 0.42 mmol, 58%, 80% purity) as a brown solid. LC/MS: RT=1.202 min, m/z [M+H]⁺=494.35.

### 3-(5-chloro-2-(((3S,4R)-3-hydroxytetrahydro-2H-pyran-4-yl)amino)pyrimidin-4-yl)-5,6,7,8-tetrahydro-4H-pyrazolo[1,5-a]azepin-4-ol

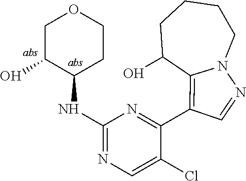 [SMILES: OC1CCCCn2ncc(-c3nc(N[C@@H]4CCOC[C@H]4O)ncc3Cl)c21]

Step 10. To a mixture of (3S,4R)-4-((4-(4-((tert-butyldimethylsilyl)oxy)-5,6,7,8-tetrahydro-4H-pyrazolo[1,5-a]azepin-3-yl)-5-chloropyrimidin-2-yl)amino)tetrahydro-2H-pyran-3-ol (260 mg, 0.53 mmol) in ACN (3 mL) was added p-toluenesulfonic acid (596 mg, 3.46 mmol) in portions at 0° C. under nitrogen atmosphere, and the resulting mixture was stirred at 50° C. for 12 hours. The crude product was concentrated in vacuo, and the resulting residue was purified by reverse-phase flash chromatography (column: C18 silica gel; mobile phase: ACN in water; gradient: 0% to 60% in 20 min; wavelength: 254 nm). The desired fractions were combined, and then concentrated in vacuo to afford 3-(5-chloro-2-(((3S,4R)-3-hydroxytetrahydro-2H-pyran-4-yl)amino)pyrimidin-4-yl)-5,6,7,8-tetrahydro-4H-pyrazolo[1,5-a]azepin-4-ol (82 mg, 0.15 mmol, 30%) as a colorless solid. LC/MS: RT=0.598 min, m/z [M+H]⁺=380.10.

### (S)-3-(5-chloro-2-(((3S,4R)-3-hydroxytetrahydro-2H-pyran-4-yl)amino)pyrimidin-4-yl)-5,6,7,8-tetrahydro-4H-pyrazolo[1,5-a]azepin-4-ol and (R)-3-(5-chloro-2-(((3S,4R)-3-hydroxytetrahydro-2H-pyran-4-yl)amino)pyrimidin-4-yl)-5,6,7,8-tetrahydro-4H-pyrazolo[1,5-a]azepin-4-ol

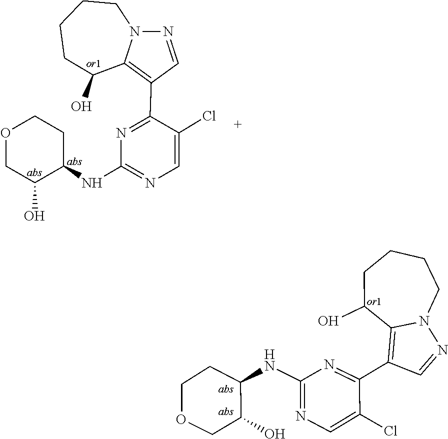 [SMILES: O[C@@H]1COCC[C@H]1Nc1ncc(Cl)c(-c2cnn3c2[C@@H](O)CCCC3)n1 ; OC1CCCCn2ncc(-c3nc(N[C@@H]4CCOC[C@H]4O)ncc3Cl)c21]

Step 11. The compound 3-(5-chloro-2-(((3S,4R)-3-hydroxytetrahydro-2H-pyran-4-yl)amino)pyrimidin-4-yl)-5,6,7,8-tetrahydro-4H-pyrazolo[1,5-a]azepin-4-ol (41 mg, 0.11 mmol) was purified by prep chiral HPLC (column: CHIRALPAK IG, 2×25 cm, 5 μm; mobile phase A: hexanes (0.2% TEA), HPLC grade; mobile phase B: EtOH/DCM (1:1), HPLC grade; flow rate: 20 mL/min; gradient: 50% B to 50% B in 14 min; wavelength: 220 nm & 254 nm; RT1 (min): 6.18; RT2 (min): 10.30; sample solvent: EtOH/DCM (1:1), HPLC grade; injection volume: 0.7 mL; number of runs: 3). The desired fractions were combined, and then concentrated in vacuo to afford I-86 (21.7 mg, 55 mol, 51%, 97% purity) as a white amorphous solid. LC/MS: RT=1.185 min, m/z [M+H]⁻=380.10. The desired fractions were combined, and then concentrated in vacuo to afford I-87 (24.2 mg, 63 μmol, 58%, 99% purity) as a white amorphous solid. LC/MS: RT=1.132 min, m/z [M+H]⁺=380.10.

### Example 22

### rac-(1R,2S,3S)-2-((tert-butyldimethylsilyl)oxy)-3-isopropoxycyclohexan-1-amine (Synthetic Intermediate)

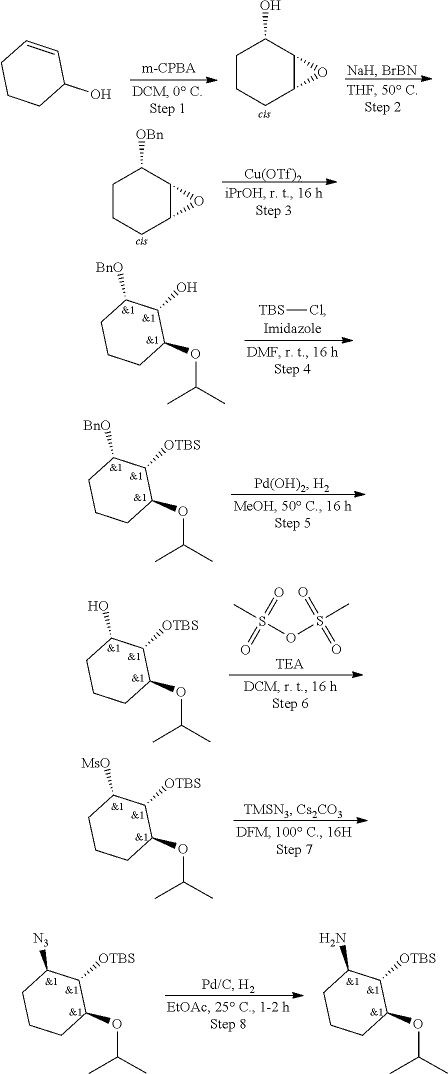

### cis-7-oxabicyclo[4.1.0]heptan-2-ol

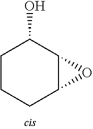

Step 1. A round-bottom flask was charged with cyclohex-2-en-1-ol (5 g, 0.05 mol), a stir bar and DCM (200 mL). Next, m-CPBA (40 g, 0.2 mol) was added at 0° C., and the solution was stirred at 0° C. for 3 hour. The reaction mixture was filtered through a pad of Celite, the pad was washed with DCM and then with hexane, and the combined filtrates were concentrated in vacuo to afford crude cis-7-oxabicyclo[4.1.0]heptan-2-ol (9.5 g, 83 mmol, 200%) as a white oil. This crude product was used in the next step without further purification. LC/MS: RT=0.100 min, m/z [M+H+ACN]⁺=156.05.

### cis-2-(benzyloxy)-7-oxabicyclo[4.1.0]heptane

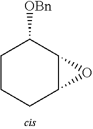 [SMILES: c1ccc(CO[C@H]2CCC[C@H]3O[C@@H]23)cc1]

Step 2. To a stirring solution of NaH (6.7 g, 60% wt, 0.17 mol) in THF (80 mL) at 0° C. was added (bromomethyl)benzene (16 g, 92 mmol). Next, crude cis-7-oxabicyclo[4.1.0]heptan-2-ol (9.5 g, 83 mmol) (crude) was added at 50° C., and the reaction was stirred at 50° C. for 16 hours with monitoring by LC/MS. The mixture was quenched with aq. NH₄Cl and extracted with EtOAc (3×200 mL), and the combined organic layers were washed with brine, dried over sodium sulfate, and then concentrated in vacuo. The crude product was purified by silica gel chromatography (10 g column), eluting with PE/EtOAc (5:1). The desired fractions were combined, and then concentrated in vacuo to afford cis-2-(benzyloxy)-7-oxabicyclo[4.1.0]heptane (4.3 g, 17 mmol, 20%, 80.81% purity) as a yellow oil. LC/MS: RT=0.880 min, m/z [M+H+ACN]⁺=246.15.

### rac-(1R,2R,6R)-2-(benzyloxy)-6-isopropoxycyclohexan-1-ol

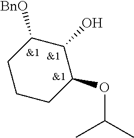 [SMILES: CC(C)O[C@H]1CCC[C@H](OCc2ccccc2)[C@@H]1O]

Step 3. A round-bottom flask was charged with cis-2-(benzyloxy)-7-oxabicyclo[4.1.0]heptane (4.3 g, 21 mmol), a solution of copper(II)trifluoromethanesulfonate (7.6 g, 21 mmol) in WA (40 mL), and a stir bar, and the resulting solution was stirred at 18° C. for 16 hours. The reaction mixture was diluted with water (40 mL), the aq. phase was extracted with EtOAc (3×60 mL), and the combined organic layers were washed with brine, dried over sodium sulfate, filtered, and then concentrated in vacuo. The crude product was purified by silica gel chromatography, eluting with PE/EtOAc (5:1). The desired fractions were combined, and then concentrated in vacuo to afford rac-(1R,2R,6R)-2-(benzyloxy)-6-isopropoxycyclohexan-1-ol (3.2 g, 10 mmol, 48%, 82.8% purity) as a white oil. LC/MS: RT=0.963 min, m/z [M+H]⁺=265.2.

### rac-(((1R,2R,6R)-2-(benzyloxy)-6-isopropoxycyclohexyl)oxy)(tert-butyl)dimethylsilane

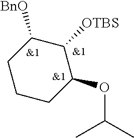 [SMILES: CC(C)O[C@H]1CCC[C@H](OCc2ccccc2)[C@@H]1O[Si](C)(C)C(C)(C)C]

Step 4. A round-bottom flask was charged with a solution of rac-(1R,2R,6R)-2-(benzyloxy)-6-isopropoxycyclohexan-1-ol (4 g, 0.02 mol) in DMF (40 mL), and a stir bar. Next, imidazole (1 g, 1 mL, 0.02 mol) and TBS-C₁ (2 g, 0.02 mol) were added at 0° C., and the resulting solution was stirred at 18° C. for 16 hours. The reaction mixture was diluted with water (40 mL), the aq. phase was extracted with EtOAc (3×60 mL), and the combined organic layers were washed with brine, dried over sodium sulfate, filtered, and then concentrated in vacuo. The crude product was purified by silica gel chromatography, eluting with PE/EtOAc (20:1). The desired fractions were combined, and then concentrated in vacuo to afford rac-(((1R,2R,6R)-2-(benzyloxy)-6-isopropoxycyclohexyl)oxy)(tert-butyl)dimethylsilane (5.2 g, 12 mmol, 80%, 84.68% purity) as a yellow oil. LC/MS: RT=1.538 min, m/z [M+H+Na]⁺=401.25.

### rac-(1R,2R,3R)-2-((tert-butyldimethylsilyl)oxy)-3-isopropoxycyclohexan-1-ol

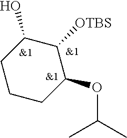 [SMILES: CC(C)O[C@H]1CCC[C@H](O)[C@@H]1O[Si](C)(C)C(C)(C)C]

Step 5. To a solution of rac-(((1R,2R,6R)-2-(benzyloxy)-6-isopropoxycyclohexyl)oxy)(tert-butyl)dimethylsilane (5 g, 0.01 mol) in MeOH (50 mL), was added palladium(II) dihydroxide (2 g, 0.01 mol) under nitrogen atmosphere. The mixture was reacted at 50° C. under hydrogen atmosphere for 16 hours, filtered through a Celite pad, and then concentrated under reduced pressure to afford crude rac-(1R,2R,3R)-2-((tert-butyldimethylsilyl)oxy)-3-isopropoxycyclohexan-1-ol (3.2 g, 11 mmol, 80%) as an off-yellow oil. LC/MS: RT=1.292 min, m/z [M+H]⁺=289.20.

### rac-(1R,2S,3R)-2-((tert-butyldimethylsilyl)oxy)-3-isopropoxycyclohexyl methanesulfonate

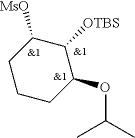 [SMILES: CC(C)O[C@H]1CCC[C@H](OS(C)(=O)=O)[C@@H]1O[Si](C)(C)C(C)(C)C]

Step 6. A round-bottom flask was charged with rac-(1R,2R,3R)-2-((tert-butyldimethylsilyl)oxy)-3-isopropoxycyclohexan-1-ol (1 g, 3 mmol), methanesulfonic anhydride (3 g, 0.02 mol), a solution of TEA (4 g, 0.03 mol) in DCM (20 mL), and a stir bar, and the resulting solution was stirred at 18° C. for 16 hours. Next, the reaction mixture was diluted with water (20 mL), the aq. phase was extracted with DCM (3×30 mL), and the combined organic layers were washed with brine, dried over sodium sulfate, filtered, and then concentrated in vacuo to afford crude rac-(1R,2S,3R)-2-((tert-butyldimethylsilyl)oxy)-3-isopropoxycyclohexyl methanesulfonate (1.3 g, 3.5 mmol, 100%) as a black oil. LC/MS: RT=1.288 min, m/z [M+H]⁺=367.25.

### rac-(((1R,2S,6R)-2-azido-6-isopropoxycyclohexyl)oxy)(tert-butyl)dimethylsilane

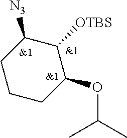 [SMILES: CC(C)O[C@H]1CCC[C@@H](N=[N+]=[N-])[C@@H]1O[Si](C)(C)C(C)(C)C]

Step 7. To a mixture of cesium carbonate (1.78 g, 5.46 mmol) and trimethylsilyl azide (471 mg, 4.09 mmol) in DMF (15 mL) was added rac-(1R,2S,3R)-2-((tert-butyldimethylsilyl)oxy)-3-isopropoxycyclohexyl methanesulfonate (500 mg, 1.36 mmol) in portions at 25° C. under nitrogen atmosphere. The mixture was stirred at 100° C. for 16 hours, with monitoring by LC/MS. Next, the mixture was cooled to r.t. and diluted with water (50 mL). The aq. phase was extracted with EtOAc (3×120 mL), and the combined organic layers were washed with brine, dried over sodium sulfate, filtered, and then concentrated in vacuo down to a volume of 10 mL. The resulting solution was used directly in the next step. LC/MS: RT=0.922 min, m/z [M+H]-=314.30.

### rac-(1R,2S,3S)-2-((tert-butyldimethylsilyl)oxy)-3-isopropoxycyclohexan-1-amine

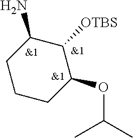 [SMILES: CC(C)O[C@H]1CCC[C@@H](N)[C@@H]1O[Si](C)(C)C(C)(C)C]

Step 8. To a solution of ac-(((1R,2S,6R)-2-azido-6-isopropoxycyclohexyl)oxy)(tert-butyl)dimethylsilane (400 mg, 1.28 mmol) in EtOAc (10 mL) was added Pd/C (40.7 mg, 383 mol) under nitrogen atmosphere, and the resulting mixture was reacted at 25° C. under hydrogen atmosphere for 1.5 hours. Next, the crude mixture was filtered through a Celite pad, and the filtrate was concentrated in vacuo to afford crude rac-(1R,2S,3S)-2-((tert-butyldimethylsilyl)oxy)-3-isopropoxycyclohexan-1-amine (310 mg, 1.08 mmol, 84.5%) as a white oil. LC/MS: RT=0.905 min, m/z [M+H]⁺=288.20.

### Example 23

### (3R,4R)-4-amino-1-isobutylcyclohexane-1,3-diol (Synthetic Intermediate)

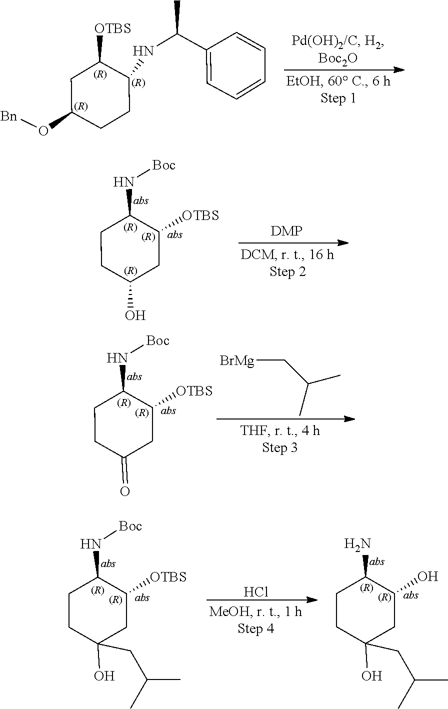

### tert-butyl ((1R,2R,4R)-2-((tert-butyldimethylsilyl)oxy)-4-hydroxycyclohexyl) carbamate

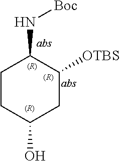

Step 1. Into a solution of (1R,2R,4R)-4-(benzyloxy)-2-((tert-butyldimethylsilyl)oxy)-N—((S)-1-phenylethyl) cyclohexan-1-amine (6 g, 0.01 mol) and Boc₂O (3 g, 0.01 mol) in EtOH (70 mL) was bubbled nitrogen (×3). Next, Pd(OH)₂/C (6 g, 0.04 mol) was added, and into the resulting mixture was bubbled hydrogen (×3). Then, the reaction mixture was stirred at 60° C. under hydrogen atmosphere for 6 hours, with monitoring by LC/MS. The mixture was filtered, and the filtrate was concentrated in vacuo to afford tert-butyl ((1R,2R,4R)-2-((tert-butyldimethylsilyl)oxy)-4-hydroxycyclohexyl) carbamate (4.2 g, 12 mmol, 90%) as a white solid. LC/MS: RT=0.908 min, m/z [M+H]⁺=346.20.

### tert-butyl ((1R,2R)-2-((tert-butyldimethylsilyl)oxy)-4-oxocyclohexyl)carbamate

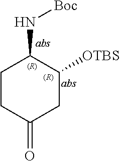

Step 2. To a mixture of tert-butyl ((1R,2R,4R)-2-((tert-butyldimethylsilyl)oxy)-4-hydroxycyclohexyl)carbamate (2 g, 6 mmol) in DCM (20 mL) was added Dess-Martin periodinane (6 g, 4 mL, 0.01 mol) in portions at 15° C. under nitrogen atmosphere, and the resulting mixture was stirred at 15° C. for 16 hours. Next, the mixture was diluted with H₂O (30 mL), the aq. phase was extracted with EtOAc (3×30 mL), and the combined organic layers were washed with brine, dried over sodium sulfate, filtered, and then concentrated in vacuo. The residue was purified by silica gel chromatography, eluting with PE/EtOAc (2:1). The desired fractions were combined, and then concentrated in vacuo to afford crude tert-butyl ((1R,2R)-2-((tert-butyldimethylsilyl)oxy)-4-oxocyclohexyl)carbamate (1.7 g, 4.9 mmol, 90%) as an off-yellow oil. LC/MS: RT=1.378 min, m/z [M+H-tBu]⁺=288.15.

### tert-butyl ((1R,2R)-2-((tert-butyldimethylsilyl)oxy)-4-hydroxy-4-isobutylcyclohexyl)carbamate

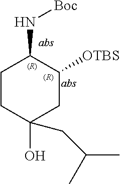

To a mixture of lanthanum(III) chloride bis(lithium chloride) (2.3 g, 7.0 mmol) in THE (20 mL) was added isobutylmagnesium bromide (1.1 g, 7.0 mmol) in portions at 0° C. under nitrogen atmosphere, and the resulting mixture was stirred at 15° C. for 3 hours. Next, tert-butyl ((1R,2R)-2-((tert-butyldimethylsilyl)oxy)-4-oxocyclohexyl)carbamate (1.6 g, 4.7 mmol) was added, and the reaction mixture was stirred at 15° C. for 4 hours. The solution was quenched with sat'd. (aq.) NH₄Cl, diluted with H₂O (30 mL) and the aq. phase was extracted with EtOAc (3×30 mL). The combined organic layers were washed with brine, dried over sodium sulfate, filtered, and then concentrated in vacuo. The solution was purified by reverse-phase flash chromatography (column: C18 silica gel; mobile phase: ACN in water; gradient: 60% to 90% in 15 min). The desired fractions were combined, and then lyophilized to afford crude tert-butyl ((1R,2R)-2-((tert-butyldimethylsilyl)oxy)-4-hydroxy-4-isobutylcyclohexyl)carbamate (450 mg, 1.12 mmol, 24%) as an off-white oil. LC/MS: RT=1.317 min, m/z [M+H]⁺=402.15.

### (3R,4R)-4-amino-1-isobutylcyclohexane-1,3-diol

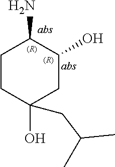

Step 4. To a mixture of tert-butyl ((1R,2R)-2-((tert-butyldimethylsilyl)oxy)-4-hydroxy-4-isobutylcyclohexyl) carbamate (440 mg, 1.10 mmol) was added a solution of hydrogen chloride in MeOH (1 mL, 4 N), and the resulting mixture was stirred at 15° C. for 1 hour. Concentration in vacuo afforded (3R,4R)-4-amino-1-isobutylcyclohexane-1,3-diol as a crude residue that was used directly in further chemistry, without any purification. LC/MS: RT=0.558 min, m/z [M+H]⁺=188.20.

### Example 24

### (R)-2-(6-(5-chloro-2-((4-(methylsulfonyl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol and (S)-2-(6-(5-chloro-2-((4-(methylsulfonyl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol (Compounds I-431 and I-435)

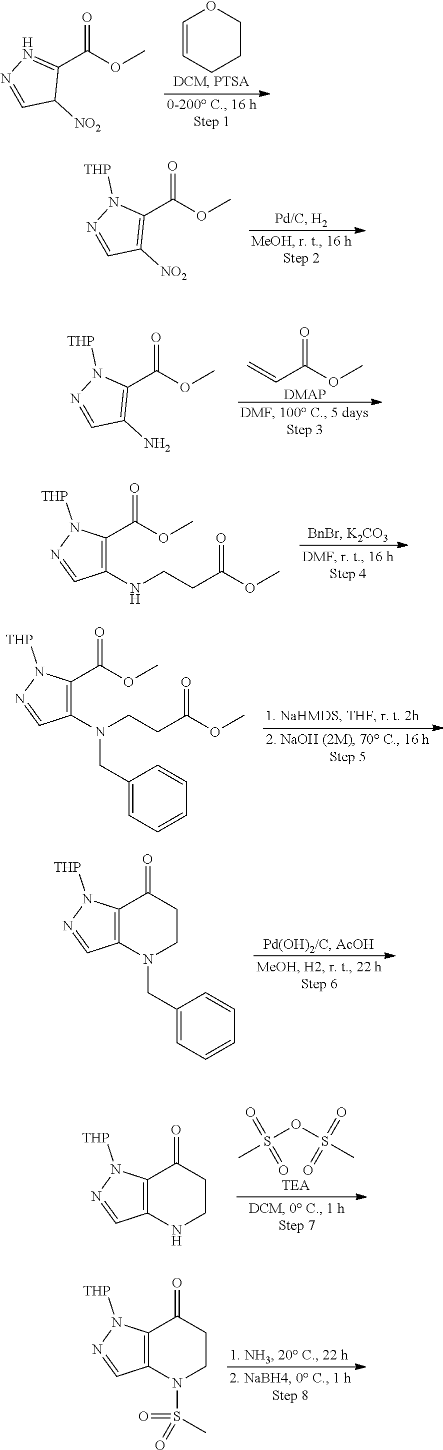

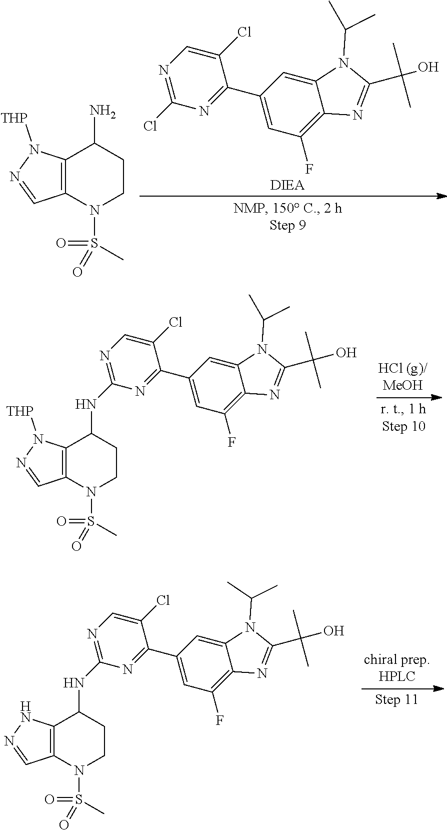

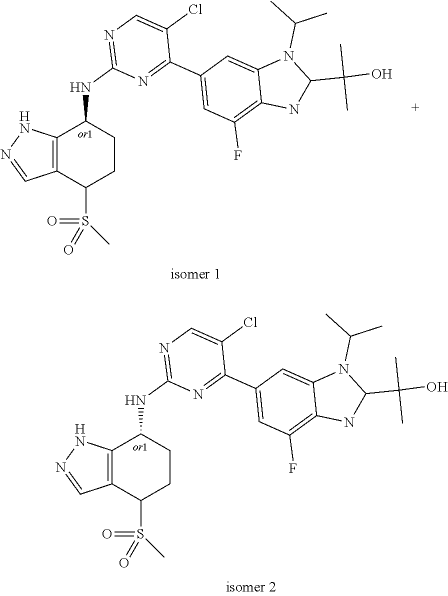 [SMILES: CC(C)N1c2cc(-c3nc(N[C@H]4CCC(S(C)(=O)=O)c5cn[nH]c54)ncc3Cl)cc(F)c2NC1C(C)(C)O ; CC(C)N1c2cc(-c3nc(N[C@@H]4CCC(S(C)(=O)=O)c5cn[nH]c54)ncc3Cl)cc(F)c2NC1C(C)(C)O]

### methyl 4-nitro-1-(tetrahydro-2H-pyran-2-yl)-1H-pyrazole-5-carboxylate

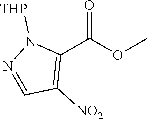

Step 1. To a mixture of methyl 4-nitro-1H-pyrazole-5-carboxylate (20 g, 0.12 mol) and Ts-OH (2.2 g, 0.012 mol) in DCM (200 mL) was added DHP (12 g, 0.14 mol) dropwise at 0° C. under nitrogen atmosphere, and the resulting mixture was stirred first at 0° C. for 1 h, and then at 20° C. for 16 hours. The crude product was purified by silica gel chromatography, eluting with PE/EtOAc (4:1). The desired fractions were combined, and then concentrated in vacuo to afford methyl 4-nitro-1-(tetrahydro-2H-pyran-2-yl)-1H-pyrazole-5-carboxylate (27 g, 99 mmol, 85%, 94% purity) as a yellow oil. LC/MS: RT=1.010 min, m/z [M+H]⁺=256.05.

### methyl 4-amino-1-(tetrahydro-2H-pyran-2-yl)-1H-pyrazole-5-carboxylate

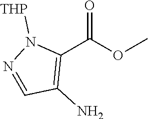

Step 2. To a solution of methyl 4-nitro-1-(tetrahydro-2H-pyran-2-yl)-1H-pyrazole-5-carboxylate (27 g, 0.11 mol) in MeOH (300 mL) was added Pd/C (10%, 3.4 g) under nitrogen atmosphere. Next, the mixture was hydrogenated at room temperature under hydrogen atmosphere for 16 hours. The reaction mixture was filtered through a pad of Celite, the pad was washed with EtOAc, and the filtrate was concentrated in vacuo to afford methyl 4-amino-1-(tetrahydro-2H-pyran-2-yl)-1H-pyrazole-5-carboxylate (25 g, 0.10 mol, 99%, 94% purity) as a purple oil. LC/MS: RT=0.663 min, m/z [M+H]⁺=226.05.

### methyl 4-((3-methoxy-3-oxopropyl) amino)-1-(tetrahydro-2H-pyran-2-yl)-1H-pyrazole-5-carboxylate

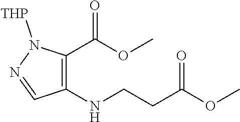

Step 3. To a mixture of methyl 4-amino-1-(tetrahydro-2H-pyran-2-yl)-1H-pyrazole-5-carboxylate (15 g, 67 mmol) and DMAP (0.81 g, 6.7 mmol) in DMF (150 mL) was added methyl acrylate (57 g, 670 mmol) dropwise at 20° C. under nitrogen atmosphere, and the resulting mixture was stirred at 100° C. for 5 days. Next, the mixture was diluted with water (300 mL), the aq. phase was extracted with EtOAc (3×500 mL), and the combined organic layers were washed with brine, dried over sodium sulfate, filtered, and then concentrated in vacuo. The crude product was purified by silica gel chromatography (column: 330 g), eluting with PE/EtOAc (2:1). The desired fractions were combined, and then concentrated in vacuo to afford methyl 4-((3-methoxy-3-oxopropyl) amino)-1-(tetrahydro-2H-pyran-2-yl)-1H-pyrazole-5-carboxylate (11.1 g, 27 mmol, 40%, 75% purity) as a yellow oil. LC/MS: RT=0.787 min, m/z [M+H]⁺=312.05.

### methyl 4-(benzyl(3-methoxy-3-oxopropyl)amino)-1-(tetrahydro-2H-pyran-2-yl)-1H-pyrazole-5-carboxylate

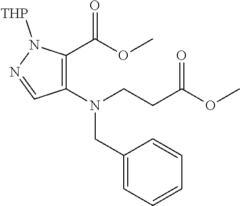

Step 4. To a mixture of methyl 4-((3-methoxy-3-oxopropyl)amino)-1-(tetrahydro-2H-pyran-2-yl)-1H-pyrazole-5-carboxylate (11 g, 35 mmol) and K₂CO₃ (9.8 g, 71 mmol) in DMF (110 mL), was added benzyl bromide (7.3 g, 42 mmol) dropwise at 20° C. under nitrogen atmosphere, and the resulting mixture was stirred at 20° C. for 16 hours. The mixture was diluted with water (150 mL), the aq. phase was extracted with EtOAc (3×250 mL) three times, and the combined organic layers were washed with brine, dried over sodium sulfate, filtered, and then concentrated in vacuo. The crude product was purified by silica gel chromatography, eluting with PE/EtOAc (3:1). The desired fractions were combined, and then concentrated in vacuo to afford methyl 4-(benzyl(3-methoxy-3-oxopropyl)amino)-1-(tetrahydro-2H-pyran-2-yl)-1H-pyrazole-5-carboxylate (13.3 g, 27 mmol, 77%, 82% Purity) as a yellow oil. LC/MS: RT=1.158 min, m/z [M+H]⁺=402.15.

### 4-benzyl-1-(tetrahydro-2H-pyran-2-yl)-1,4,5,6-tetrahydro-7H-pyrazolo[4,3-b]pyridin-7-one

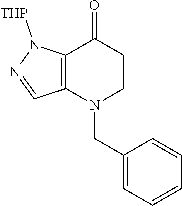

Step 5. To a mixture of methyl 4-(benzyl(3-methoxy-3-oxopropyl)amino)-1-(tetrahydro-2H-pyran-2-yl)-1H-pyrazole-5-carboxylate (6 g, 14.5 mmol) in THF (60 mL) was added NaHMDS (11 mL, 2 M in THF) dropwise at 20° C. under nitrogen atmosphere, and the resulting mixture was stirred at 20° C. for 2 hours. Next, the reaction was treated with NaOH (70 mL, 2 M in H₂O), and the resulting mixture was stirred at 70° C. for 16 hours. The reaction mixture was quenched with water, and its pH was adjusted to 7-8 with hydrochloric acid (2 M). The mixture was diluted with water (50 mL), the aq. phase was extracted with EtOAc (3×200 mL), and the combined organic layers were washed with brine, dried over sodium sulfate, filtered, and then concentrated in vacuo. The crude product was purified by silica gel chromatography, eluting with PE/EtOAc (2:1). The desired fractions were combined, and then concentrated in vacuo to afford 4-benzyl-1-(tetrahydro-2H-pyran-2-yl)-1,4,5,6-tetrahydro-7H-pyrazolo[4,3-b]pyridin-7-one (1.95 g, 6.26 mmol, 40%, 97% purity) as a yellow amorphous solid. LC/MS: RT=1.078 min, m/z [M+H]⁺=312.10.

### 1-(tetrahydro-2H-pyran-2-yl)-1,4,5,6-tetrahydro-7H-pyrazolo[4,3-b]pyridin-7-one

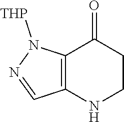

Step 6. To a solution of 4-benzyl-1-(tetrahydro-2H-pyran-2-yl)-1,4,5,6-tetrahydro-7H-pyrazolo[4,3-b]pyridin-7-one (1.95 g, 6.26 mmol) and acetic acid (5.26 g, 87.7 mmol) in MeOH (20 mL) was added palladium(II) hydroxide on carbon (20%, 264 mg) under nitrogen atmosphere, and the resulting mixture was hydrogenated at room temperature under hydrogen atmosphere for 22 hours. The reaction mixture was filtered through a pad of Celite, the pad was washed with EtOAc, and the filtrate was concentrated in vacuo to afford crude 1-(tetrahydro-2H-pyran-2-yl)-1,4,5,6-tetrahydro-7H-pyrazolo[4,3-b]pyridin-7-one (1.5 g, 4.7 mmol, 76%, 70% purity) as a yellow oil. LC/MS: RT=0.438 min, m/z [M+H]⁺=222.10.

### 4-(methylsulfonyl)-1,4,5,6-tetrahydro-7H-pyrazolo[4,3-b]pyridin-7-one

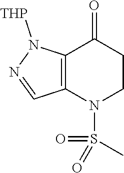

Step 7. To a mixture of 1-(tetrahydro-2H-pyran-2-yl)-1,4,5,6-tetrahydro-7H-pyrazolo[4,3-b]pyridin-7-one (1 g, 5 mmol) and TEA (1 g, 10 mmol) in DCM (10 mL), was added methanesulfonic anhydride (1 g, 7 mmol) dropwise at 0° C. under nitrogen atmosphere, and the resulting mixture was stirred at 0° C. for 1 hour. Next, the mixture was concentrated in vacuo, and then purified by reverse-phase flash chromatography (mobile phase ACN/water; gradient: 20% to 70% in 15 min, wavelength: 254 nm). The desired fractions were combined, and then concentrated in vacuo to afford 4-(methylsulfonyl)-1,4,5,6-tetrahydro-7H-pyrazolo[4,3-b]pyridin-7-one (600 mg, 2.3 mmol, 50%, 83% purity) as a yellow amorphous solid. LC/MS: RT=0.283 min, m/z [M+H]⁺=299.95.

### 4-(methylsulfonyl)-1-(tetrahydro-2H-pyran-2-yl)-4,5,6,7-tetrahydro-1H-pyrazolo[4,3-b]pyridin-7-amine

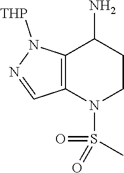

Step 8. A mixture of 4-(methylsulfonyl)-1-(tetrahydro-2H-pyran-2-yl)-1,4,5,6-tetrahydro-7H-pyrazolo[4,3-b]pyridin-7-one (850 mg, 2.84 mmol) and ammonia (10 mL, 7 M in MeOH) was stirred at 20° C. for 22 hours. Next, NaBH₄ (129 mg, 3.41 mmol) was added in portions at 0° C., and the resulting mixture was stirred at 0° C. for 1 hour. Next, the reaction mixture was quenched with 10H₂O·Na₂SO₄ at 0° C., and then stirred at 0° C. for 1 hour. Then, it was filtered through a pad of Celite, the pad was washed with MeOH, and the filtrate was concentrated in vacuo to afford crude 4-(methylsulfonyl)-1-(tetrahydro-2H-pyran-2-yl)-4,5,6,7-tetrahydro-1H-pyrazolo[4,3-b]pyridin-7-amine (820 mg, 1.4 mmol, 48%, 50% purity) as a yellow amorphous solid. LC/MS: RT=0.667 min, m/z [M+H]⁺=301.10.

### 2-(6-(5-chloro-2-((4-(methylsulfonyl)-1-(tetrahydro-2H-pyran-2-yl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol

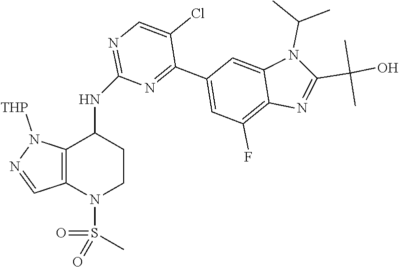

Step 9. To a mixture of 4-(methylsulfonyl)-1-(tetrahydro-2H-pyran-2-yl)-4,5,6,7-tetrahydro-1H-pyrazolo[4,3-b]pyridin-7-amine (800 mg, 50% wt, 1.33 mmol) and DIEA (516 mg, 3.99 mmol) in NMP (8 mL) was added 2-(6-(2,5-dichloropyrimidin-4-yl)-4-fluoro-1-isopropyl-1H-benzo[d]imidazol-2-yl)propan-2-ol (255 mg, 0.67 mmol) in portions at 20° C. under nitrogen atmosphere. The resulting crude material was purified by reverse-phase flash chromatography (mobile phase: ACN/water/0.1% aq. NH₄HCO₃, gradient: 20% to 70% in 15 min; wavelength: 254 nm). The desired fractions were combined, and then concentrated in vacuo to afford 2-(6-(5-chloro-2-((4-(methylsulfonyl)-1-(tetrahydro-2H-pyran-2-yl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol (140 mg, 0.15 mmol, 11%, 70% purity) as a yellow amorphous solid. LC/MS: RT=0.872 min, m/z [M+H]⁺=647.35.

### 2-(6-(5-chloro-2-((4-(methylsulfonyl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol

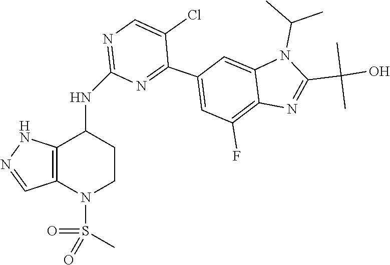 [SMILES: CC(C)n1c(C(C)(C)O)nc2c(F)cc(-c3nc(NC4CCN(S(C)(=O)=O)c5cn[nH]c54)ncc3Cl)cc21]

Step 10. To a mixture of 2-(6-(5-chloro-2-((4-(methylsulfonyl)-1-(tetrahydro-2H-pyran-2-yl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol (130 mg, 201 μmol) was added HCl (g)/MeOH (0.6 mL), and the resulting mixture was stirred at 20° C. for 1 hour. The mixture was diluted with water (4 mL), and its pH was adjusted to 8-9 with aq. Na₂CO₃. The aq. phase was extracted with EtOAc (3×20 mL) three times, and the combined organic layers were washed with brine, dried over sodium sulfate, filtered, and then concentrated in vacuo. The resulting crude material was purified by reverse-phase flash chromatography (mobile phase A: ACN; mobile phase B: water [0.1% NH₄HCO₃]; gradient: 20% to 70% in 15 min; wavelength: 254 nm). The desired fractions were combined, and then concentrated in vacuo to afford 2-(6-(5-chloro-2-((4-(methylsulfonyl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol (40 mg, 70 μmol, 35%, 98.3% purity) as a yellow amorphous solid. LC/MS: RT=0.998 min, m/z [M+H]⁺=563.15.

### rel-(R)-2-(6-(5-chloro-2-((4-(methylsulfonyl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol

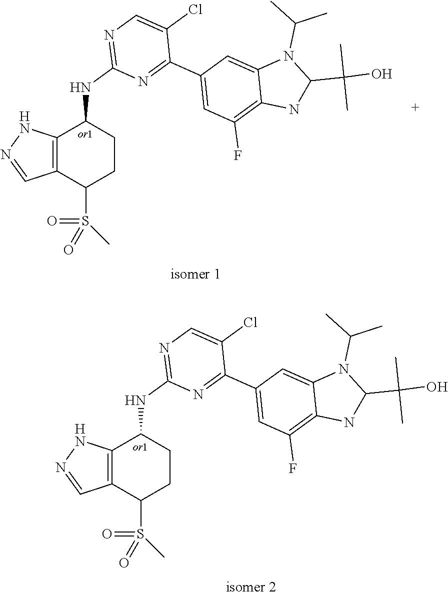 [SMILES: CC(C)N1c2cc(-c3nc(N[C@H]4CCC(S(C)(=O)=O)c5cn[nH]c54)ncc3Cl)cc(F)c2NC1C(C)(C)O ; CC(C)N1c2cc(-c3nc(N[C@@H]4CCC(S(C)(=O)=O)c5cn[nH]c54)ncc3Cl)cc(F)c2NC1C(C)(C)O]

Step 11. The resulting crude product from Step 10 was purified by chiral preparative HPLC (column: CHIRALPAK 1H, 2×25 cm, 5 μm; mobile phase A: hexanes (0.2% TEA), mobile phase B: EtOH/DCM (1:1) HPLC grade; flow rate: 20 mL/min; gradient: 30% B to 30% B in 14 min; wavelength: 220 nm & 254 nm; RTIsomer1 (min): 6.87, RTIsomer2 (min) 10.81; sample solvent: EtOH/DCM (1:1); injection volume: 0.7 mL; number of runs: 5). Isomer 1: The desired fractions were combined, and then lyophilized to afford rel-(R)-2-(6-(5-chloro-2-((4-(methylsulfonyl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol (14.6 mg, 24.1 μmol, 68%, 92.9% purity) as a white amorphous solid. LC/MS: RT=1.392 min, m/z [M+H]⁺=563.20. Isomer 2: The desired fractions were combined, and then lyophilized to afford rel-(R)-2-(6-(5-chloro-2-((4-(methylsulfonyl)-4,5,6,7-tetrahydro-1H-pyrazolo[4,3-b]pyridin-7-yl)amino)pyrimidin-4-yl)-4-fluoro-1-isopropyl-1H-benzo[d]imidazol-2-yl)propan-2-ol (11 mg, 19 mol, 55%, 99.3% purity) as a white amorphous solid. LC/MS: RT=0.998 min, m/z [M+H]⁺=563.15.

### Example 25

### 6-(2,5-dichloropyrimidin-4-yl)-4-fluoro-1-isopropyl-N,N-dimethyl-1H-benzo[d]imidazol-2-amine (Synthetic Intermediate)

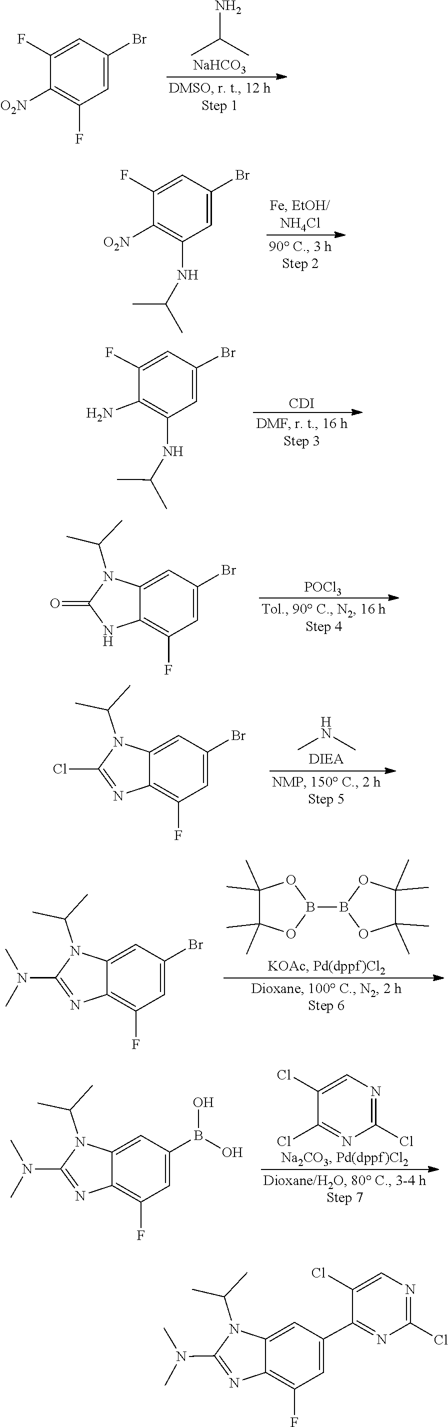

5-bromo-3-fluoro-N-isopropyl-2-nitroaniline

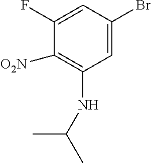

Step 1. A solution of 5-bromo-1,3-difluoro-2-nitrobenzene (10 g, 42 mmol) in DMSO (150 mL) was cooled in an ice bath and then treated with sodium bicarbonate (3.5 g, 42 mmol), and the resulting mixture was stirred. Next, propan-2-amine (2.5 g, 42 mmol) was slowly added dropwise, and then the reaction was allowed to warm up to r.t. and reacted overnight. The solids were filtered off, and the combined filtrates were concentrated in vacuo to afford crude 5-bromo-3-fluoro-N-isopropyl-2-nitroaniline (10 g, 30 mmol, 70%, 82% purity) as a light brown solid, which was used directly in the next step. LC/MS: RT=1.330 min, m/z [M+H]⁺=277.05.

### 5-bromo-3-fluoro-N1-isopropylbenzene-1,2-diamine

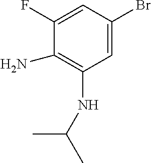

Step 2. A reaction vial was charged with a solution of 5-bromo-3-fluoro-N-isopropyl-2-nitroaniline (10 g, 36 mmol) in EtOH (200 mL) and water (50.0 mL), followed by iron (13 g, 0.23 mol) and ammonium chloride (4.4 g, 83 mmol). The resulting mixture was stirred at 90° C. under nitrogen atmosphere for 3 hours, with monitoring by LC/MS. The mixture was cooled to r.t., and then filtered through a pad of Celite, and the pad was washed with EtOH. The combined filtrates were concentrated in vacuo, the pH of the residue was adjusted to 7 with aq. NaHCO₃, and the aq. phase was extracted with EtOAc (3×200 mL). The combined organic layers were washed with brine, dried over sodium sulfate, filtered, and then concentrated in vacuo to afford crude 5-bromo-3-fluoro-N1-isopropylbenzene-1,2-diamine (8.5 g, 28 mmol, 76%, 80% purity) as a brown oil. LC/MS: RT=0.905 min, m/z [M+H]⁺=247.00.

### 6-bromo-4-fluoro-1-isopropyl-1,3-dihydro-2H-benzo[d]imidazol-2-one

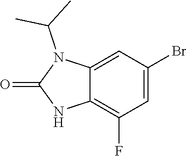

Step 3. A reaction vial was charged with 5-bromo-3-fluoro-N1-isopropylbenzene-1,2-diamine (8.5 g, 34 mmol) in DMF (32 mL), and a stir bar, and then CDI (5.6 g, 34 mmol) was added at r.t. The resulting mixture was stirred at 25° C. for 16 hours, with monitoring by LC/MS. The reaction mixture was diluted with water (50 mL), the aq. phase was extracted with EtOAc (3×100 mL) and the combined organic layers were washed with brine, dried over sodium sulfate, filtered, and then concentrated in vacuo to afford crude 6-bromo-4-fluoro-1-isopropyl-1,3-dihydro-2H-benzo[d]imidazol-2-one (9 g, 0.03 mol, 80%, 80% purity) as a brown oil, which was used directly in the next step. LC/MS: RT=0.901 min, m/z [M−H]—=270.90.

### 6-bromo-2-chloro-4-fluoro-1-isopropyl-1H-benzo[d]imidazole

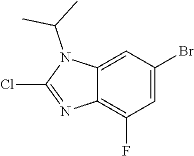

Step 4. To a mixture of 6-bromo-4-fluoro-1-isopropyl-1,3-dihydro-2H-benzo[d]imidazol-2-one (9 g, 0.03 mol) in toluene (45 mL) was added POCl₃ (45 mL) dropwise at 25° C. under nitrogen atmosphere, and the resulting mixture was stirred at 90° C. for 16 hours, with monitoring by LC/MS. Next, the mixture was cooled to r.t., and then poured into a pre-cooled (in an ice-bath) solution of sat'd. aq. sodium bicarbonate and EtOAc (150-200 mL). The resulting mixture was stirred for 10 minutes until it became clearly biphasic. The organic phase was concentrated in vacuo, and the resulting residue was purified by column chromatography, eluting with PE/EtOAc (20:1), to afford 6-bromo-2-chloro-4-fluoro-1-isopropyl-1H-benzo[d]imidazole (6.5 g, 19 mmol, 70%, 85% purity) as an off-white solid. LC/MS: RT=1.230 min, m/z [M+H]⁺=291.00.

### 6-bromo-4-fluoro-1-isopropyl-N,N-dimethyl-1H-benzo[d]imidazol-2-amine

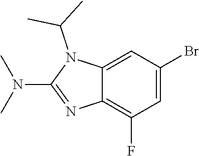 [SMILES: CC(C)n1c(N(C)C)nc2c(F)cc(Br)cc21]

Step 5. A round-bottom flask was charged with dimethylamine HCl (280 mg, 3.43 mmol), 6-bromo-2-chloro-4-fluoro-1-isopropyl-1H-benzo[d]imidazole (500 mg, 1.71 mmol), DIEA (887 mg, 6.86 mmol), NMW (3 mL) and a stir bar, and the resulting solution was stirred at 150° C. for 2 hours. Next, the solution was cooled to r.t., and then purified by reverse-phase flash chromatography (column: C18 silica gel; mobile phase: ACN in water; gradient: 10% to 80% in 20 min; wavelength: 254 nm). The desired fractions were combined, and then concentrated in vacuo to afford 6-bromo-4-fluoro-1-isopropyl-N,N-dimethyl-1H-benzo[d]imidazol-2-amine (500 mg, 1.5 mmol, 87%, 90% purity) as a light-yellow solid. LC/MS: RT=0.736 min, m/z [M+H]⁺=299.95. 4-fluoro-1-isopropyl-N,N-dimethyl-6-(4,4,5,5-tetramethyl-1,3,2-dioxaborolan-2-yl)-1H-benzo[d]imidazol-2-amine

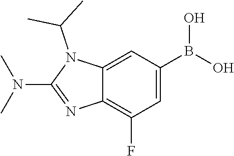 [SMILES: CC(C)n1c(N(C)C)nc2c(F)cc(B(O)O)cc21]

Step 6. To a stirring solution of 6-bromo-4-fluoro-1-isopropyl-N,N-dimethyl-1H-benzo[d]imidazol-2-amine (250 mg, 833 μmol), 4,4,4′,4′,5,5,5′,5′-octamethyl-2,2′-bi(1,3,2-dioxaborolane) (317 mg, 1.25 mmol) and potassium acetate (245 mg, 2.50 mmol) in 1,4-dioxane (5 mL) was added PdCl₂(dppf)-CH₂Cl₂ adduct (68.0 mg, 83.3 μmol), and the resulting solution was stirred at 100° C. under nitrogen atmosphere for 2 hours. The mixture was cooled to r.t. and then diluted with water (10 mL), and the aq. phase was extracted with EtOAc (3×15 mL). The combined organic layers were washed with brine, dried over sodium sulfate, filtered, and then concentrated in vacuo to afford crude 4-fluoro-1-isopropyl-N,N-dimethyl-6-(4,4,5,5-tetramethyl-1,3,2-dioxaborolan-2-yl)-1H-benzo[d]imidazol-2-amine (230 mg, 662 mol, 79.5%) as a brown oil. LC/MS: RT=0.713 min, m/z [M+H]⁺=348.20.

### 6-(2,5-dichloropyrimidin-4-yl)-4-fluoro-1-isopropyl-N,N-dimethyl-1H-benzo[d]imidazol-2-amine

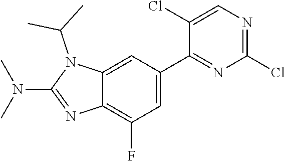 [SMILES: CC(C)n1c(N(C)C)nc2c(F)cc(-c3nc(Cl)ncc3Cl)cc21]

Step 7. To a stirring solution of 4-fluoro-1-isopropyl-N,N-dimethyl-6-(4,4,5,5-tetramethyl-1,3,2-dioxaborolan-2-yl)-1H-benzo[d]imidazol-2-amine (250 mg, 720 μmol), 2,4,5-trichloropyrimidine (158 mg, 864 μmol) and Na₂CO₃ (229 mg, 2.16 mmol) in 1,4-dioxane (3 mL) and water (0.6 mL) was added PdCl₂(dppf)-CH₂Cl₂ adduct (58.8 mg, 72.0 mol), and the resulting solution was stirred at 80° C. under nitrogen atmosphere for 4 hours. The resulting mixture was concentrated in vacuo, dissolved in DMF and the resulting solution was purified by flash chromatography (column: C18; mobile phase A: water, mobile phase B: ACN; flow rate: 60 mL/min; gradient: 0% B to 100% B in 40 min; wavelength: 220 nm & 254 nm). The desired fractions were combined, and then concentrated in vacuo to afford 6-(2,5-dichloropyrimidin-4-yl)-4-fluoro-1-isopropyl-N,N-dimethyl-1H-benzo[d]imidazol-2-amine (150 mg, 407 μmol, 56.6%) as a white solid. LC/MS: RT=0.793 min, m/z [M+H]⁺=368.00.

### Example 26

### 6-acetyl-2-chloro-8-cyclopentyl-5-methylpyrido[2,3-d]pyrimidin-7 (8H)-one (Synthetic Intermediate)

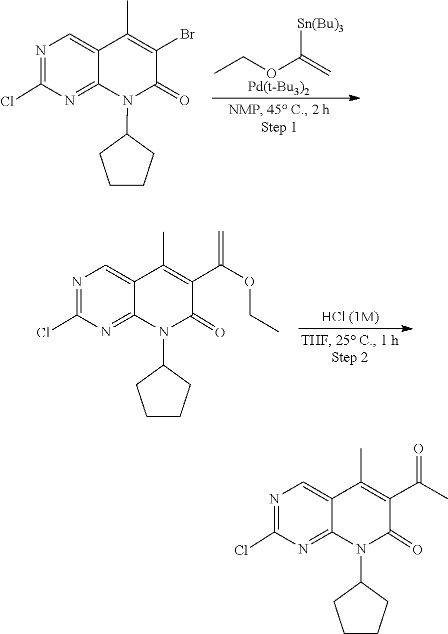

### 2-chloro-8-cyclopentyl-6-(1-ethoxyvinyl)-5-methylpyrido[2,3-d]pyrimidin-7 (8H)-one

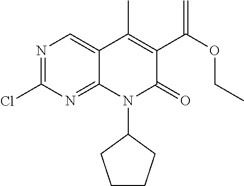 [SMILES: C=C(OCC)c1c(C)c2cnc(Cl)nc2n(C2CCCC2)c1=O]

Step 1. A round-bottom flask was charged with 6-bromo-2-chloro-8-cyclopentyl-5-methylpyrido[2,3-d]pyrimidin-7 (8H)-one (5 g, 1 eq., 0.01 mol), tributyl(1-ethoxyvinyl)stannane (6 g, 1.2 eq., 0.02 mol), Pd(t-Bu₃)₂ (0.3 g, 0.05 eq., 0.7 mmol), and a stir bar. Next, NMP (50 mL) was added, and the resulting solution was stirred at 45° C. for 2 hours. The reaction mixture was diluted with H₂O (30 mL), the aq. phase was extracted with EtOAc (3×60 mL), and the combined organic layers were washed with brine, dried over sodium sulfate, filtered, and then concentrated in vacuo. The resulting solution was purified by flash chromatography (column: using C18; mobile phase A: water; mobile phase B: ACN; flow rate: 60 mL/min; gradient: 0% B to 100% B in 40 min; wavelength: 220 nm & 254 nm). The desired fractions were combined, and then lyophilized to afford 2-chloro-8-cyclopentyl-6-(1-ethoxyvinyl)-5-methylpyrido[2,3-d]pyrimidin-7 (8H)-one (3 g, 9 mmol, 60%) as an off-white amorphous solid. m/z (ES⁺) [M+H]=334.00; HPLC tR=0.942 min.

### 6-acetyl-2-chloro-8-cyclopentyl-5-methylpyrido[2,3-d]pyrimidin-7 (8H)-one

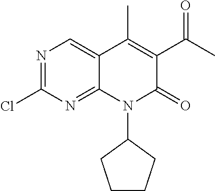 [SMILES: CC(=O)c1c(C)c2cnc(Cl)nc2n(C2CCCC2)c1=O]

Step 2. A round-bottom flask was charged with 2-chloro-8-cyclopentyl-6-(1-ethoxyvinyl)-5-methylpyrido[2,3-d]pyrimidin-7 (8H)-one (5 g, 1 eq., 0.01 mol), HCl (0.01 μL, 1M, 1 eq., 0.01 mol), and a stir bar. Next, THF (30 mL) was added, and the resulting solution was stirred at 25° C. for 1 hour. The mixture was concentrated in vacuo, and the resulting residue was purified by flash chromatography (column: using C18; mobile phase A: water; mobile phase B: ACN; flow rate: 60 mL/min; gradient: 0% B to 100% B in 40 min; wavelength: 220 nm & 254 nm). The desired fractions were combined, and then concentrated in vacuo to afford 6-acetyl-2-chloro-8-cyclopentyl-5-methylpyrido[2,3-d]pyrimidin-7 (8H)-one (2 g, 7 mmol, 40%) as an amorphous solid. m/z (ES⁺) [M+H]⁺=306.10; HPLC tR=0.872 min.

### Example 27

### N-(5-chloro-4-(4-fluoro-1-isopropyl-2-methyl-1H-benzo[d]imidazol-6-yl)pyrimidin-2-yl)-4-((4-ethylpiperazin-1-yl)methyl)-1H-indazol-7-amine (Compound I-406)

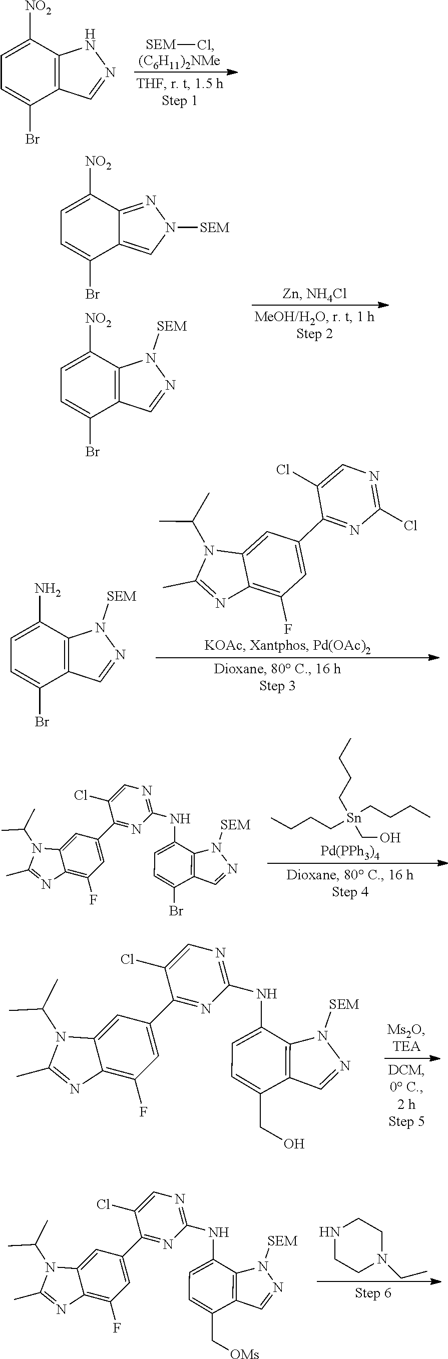

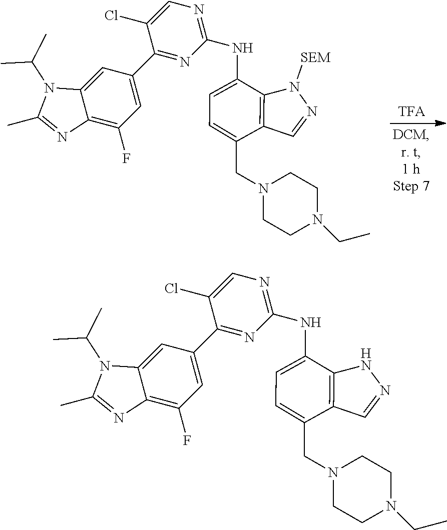

4-bromo-7-nitro-1-((2-(trimethylsilyl)ethoxy)methyl)-1H-indazole & 4-bromo-7-nitro-2-((2-(trimethylsilyl)ethoxy)methyl)-2H-indazole

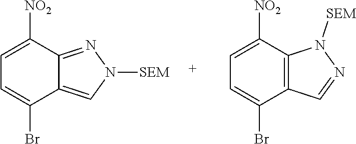 [SMILES: C[Si](C)(C)CCOCn1cc2c(Br)ccc([N+](=O)[O-])c2n1 ; C[Si](C)(C)CCOCn1ncc2c(Br)ccc([N+](=O)[O-])c21]

Step 1. A round-bottom flask was charged with 4-bromo-7-nitro-1H-indazole (1.5 g, 6.2 mmol), N-cyclohexyl-N-methylcyclohexanamine (2.5 g, 13 mmol), a solution of 2-(chloromethoxyethyl)trimethyl silane (2.2 g, 13 mmol) in THF (15 mL), and a stir bar, and the resulting solution was stirred at 25° C. for 1.5 hours. The mixture was quenched with sat'd. aq. NaHCO₃ and extracted with EtOAc. The combined organic phases were concentrated in vacuo, and the resulting residue was purified by reverse-phase flash chromatography (column: C18 silica gel; mobile phase: ACN in water; gradient: 30% to 90% in 20 min; wavelength: 254 nm). The desired fractions were combined, and then concentrated in vacuo to afford crude 4-bromo-7-nitro-1-((2-(trimethylsilyl)ethoxy)methyl)-1H-indazole (1.8 g, 4.8 mmol) as a yellow oil and crude 4-bromo-7-nitro-2-((2-(trimethylsilyl)ethoxy)methyl)-2H-indazole (0.34 g, 0.91 mmol) as yellow solid. LC/MS: RT=1.142 min, m/z [M+H]⁺=371.95.

### 4-bromo-1-((2-(trimethylsilyl)ethoxy)methyl)-1H-indazol-7-amine

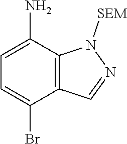 [SMILES: C[Si](C)(C)CCOCn1ncc2c(Br)ccc(N)c21]

Step 2. A round-bottom flask was charged with 4-bromo-7-nitro-1-((2-(trimethylsilyl)ethoxy)methyl)-1H-indazole (2.75 g, 7.39 mmol), zinc (2.41 g, 36.9 mmol) sat'd. aq. NH₄Cl (10 mL), MeOH (20 mL) and a stir bar, and the resulting solution was stirred at 25° C. for 1 hour. The solid was filtered off, the filtrate was concentrated in vacuo, and the resulting residue was purified by reverse-phase flash chromatography (column: C18 silica gel; mobile phase: MeOH in aq. NH₄HCO₃; gradient: 0% to 100% in 20 min; wavelength: 254 nm). The desired fractions were combined, and then concentrated in vacuo to afford 4-bromo-1-((2-(trimethylsilyl)ethoxy)methyl)-1H-indazol-7-amine (2.04 g, 5.96 mmol, 80.7%) as a brown oil. LC/MS: RT=1.527 min, m/z [M+H]⁺=342.10. 4-bromo-N-(5-chloro-4-(4-fluoro-1-isopropyl-2-methyl-1H-benzo[d]imidazol-6-yl)pyrimidin-2-yl)-1-((2-(trimethylsilyl)ethoxy)methyl)-1H-indazol-7-amine

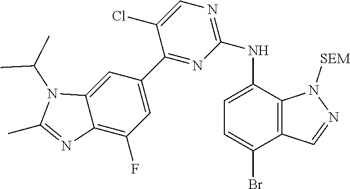 [SMILES: Cc1nc2c(F)cc(-c3nc(Nc4ccc(Br)c5cnn(COCC[Si](C)(C)C)c45)ncc3Cl)cc2n1C(C)C]

Step 3. To a solution of 4-bromo-1-((2-(trimethylsilyl)ethoxy)methyl)-1H-indazol-7-amine (1 g, 3 mmol) and 6-(2,5-dichloropyrimidin-4-yl)-4-fluoro-1-isopropyl-2-methyl-1H-benzo[d]imidazole (1 g, 3 mmol) in 1,4-dioxane (10 mL) were added KOAc (0.9 g, 9 mmol), xantphos (0.3 g, 0.6 mmol) and Pd(OAc)₂ (0.07 g, 0.3 mmol), and the resulting mixture was stirred at 80° C. under a nitrogen atmosphere for 16 hours. The reaction mixture was then concentrated in vacuo, and the resulting residue was purified by reverse-phase flash chromatography (column: C18 silica gel; mobile phase: ACN in water; gradient: 0% to 100% in 20 min; wavelength: 254 nm). The desired fractions were combined, and then concentrated in vacuo to afford 4-bromo-N-(5-chloro-4-(4-fluoro-1-isopropyl-2-methyl-1H-benzo[d]imidazol-6-yl)pyrimidin-2-yl)-1-((2-(trimethylsilyl)ethoxy)methyl)-1H-indazol-7-amine (0.94 g, 1.2 mmol, 40%, 80% purity) as a yellow solid. LC/MS: RT=1.618 min, m/z [M+H]⁺=644.30.

### (7-((5-chloro-4-(4-fluoro-1-isopropyl-2-methyl-1H-benzo[d]imidazol-6-yl)pyrimidin-2-yl)amino)-1-((2-(trimethylsilyl)ethoxy)methyl)-1H-indazol-4-yl)methanol

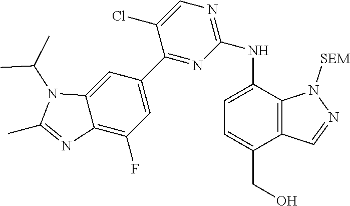 [SMILES: Cc1nc2c(F)cc(-c3nc(Nc4ccc(CO)c5cnn(COCC[Si](C)(C)C)c45)ncc3Cl)cc2n1C(C)C]

Step 4. To a solution of 4-bromo-N-(5-chloro-4-(4-fluoro-1-isopropyl-2-methyl-1H-benzo[d]imidazol-6-yl)pyrimidin-2-yl)-1-((2-(trimethylsilyl)ethoxy)methyl)-1H-indazol-7-amine (840 mg, 1.30 mmol) and (tributylstannyl)methanol (836 mg, 2.60 mmol) in 1,4-dioxane (9 mL) was added Pd(PPh₃)₄ (150 mg, 0.13 mmol), and the resulting mixture was stirred at 80° C. under a nitrogen atmosphere for 16 hours. The reaction mixture was then concentrated in vacuo, and the resulting residue was purified by reverse-phase flash chromatography (column: C18 silica gel; mobile phase: ACN in water; gradient: 0% to 100% in 20 min; wavelength: 254 nm). The desired fractions were combined, and then concentrated in vacuo to afford (7-((5-chloro-4-(4-fluoro-1-isopropyl-2-methyl-1H-benzo[d]imidazol-6-yl)pyrimidin-2-yl)amino)-1-((2-(trimethylsilyl)ethoxy)methyl)-1H-indazol-4-yl)methanol (640 mg, 0.86 mmol, 66%, 80% purity) as a yellow oil. LC/MS: RT=0.900 min, m/z [M+H]⁺=596.25.

### (7-((5-chloro-4-(1-ethyl-4-fluoro-2-methyl-1H-benzo[d]imidazol-6-yl)pyrimidin-2-yl)amino)-1-((2-(trimethylsilyl)ethoxy)methyl)-1H-indazol-4-yl)methyl methanesulfonate

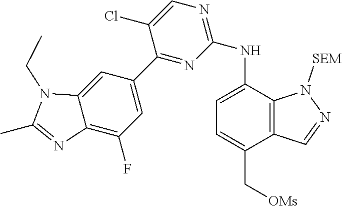 [SMILES: CCn1c(C)nc2c(F)cc(-c3nc(Nc4ccc(COS(C)(=O)=O)c5cnn(COCC[Si](C)(C)C)c45)ncc3Cl)cc21]

Step 5. A round-bottom flask was charged with (7-((5-chloro-4-(4-fluoro-1-isopropyl-2-methyl-1H-benzo[d]imidazol-6-yl)pyrimidin-2-yl)amino)-1-((2-(trimethylsilyl)ethoxy)methyl)-1H-indazol-4-yl)methanol (200 mg, 0.33 mmol), methanesulfonic anhydride (58.4 mg, 0.33 mmol), TEA (102 mg, 1.01 mmol), DCM (2 mL) and a stir bar, and the resulting solution was stirred at 0° C. for 2 hours. The crude reaction solution was used directly in the next step, without any purification. Note that the desired product could not be detected by LC/MS. N-(5-chloro-4-(4-fluoro-1-isopropyl-2-methyl-1H-benzo[d]imidazol-6-yl)pyrimidin-2-yl)-4-((4-ethylpiperazin-1-yl)methyl)-1-((2-(trimethylsilyl)ethoxy)methyl)-1H-indazol-7-amine

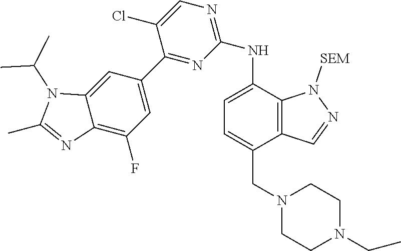 [SMILES: CCN1CCN(Cc2ccc(Nc3ncc(Cl)c(-c4cc(F)c5nc(C)n(C(C)C)c5c4)n3)c3c2cnn3COCC[Si](C)(C)C)CC1]

Step 6. 1-ethylpiperazine (203 mg, 1.78 mmol) was added directly to the crude reaction solution from the previous step, and the resulting mixture was stirred at 25° C. for 1 hour. The reaction mixture was then concentrated in vacuo, and the resulting residue was purified by reverse-phase flash chromatography (column: C18 silica gel; mobile phase: ACN in water; gradient: 0% to 100% in 20 min; wavelength: 254 nm). The desired fractions were combined, and then concentrated in vacuo to afford N-(5-chloro-4-(4-fluoro-1-isopropyl-2-methyl-1H-benzo[d]imidazol-6-yl)pyrimidin-2-yl)-4-((4-ethylpiperazin-1-yl)methyl)-1-((2-(trimethylsilyl)ethoxy)methyl)-1H-indazol-7-amine (152 mg, 0.18 mmol, 59%, 80% purity) as a yellow solid. LC/MS: RT=0.780 min, m/z [M+H]⁺=692.25. N-(5-chloro-4-(4-fluoro-1-isopropyl-2-methyl-1H-benzo[d]imidazol-6-yl)pyrimidin-2-yl)-4-((4-ethylpiperazin-1-yl)methyl)-1H-indazol-7-amine (Compound I-406)

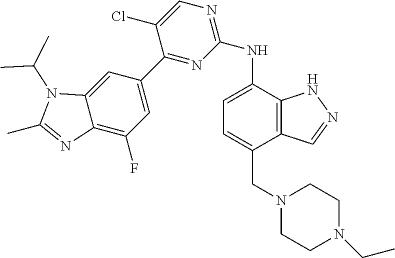 [SMILES: CCN1CCN(Cc2ccc(Nc3ncc(Cl)c(-c4cc(F)c5nc(C)n(C(C)C)c5c4)n3)c3[nH]ncc23)CC1]

Step 7. A round-bottom flask was charged with N-(5-chloro-4-(4-fluoro-1-isopropyl-2-methyl-1H-benzo[d]imidazol-6-yl)pyrimidin-2-yl)-4-((4-ethylpiperazin-1-yl)methyl)-1-((2-(trimethylsilyl)ethoxy) methyl)-1H-indazol-7-amine (142 mg, 205 μmol), TFA (1 mL), DCM (1 mL) and a stir bar, and the resulting solution was stirred at 25° C. for 1 hour. The reaction mixture was then concentrated in vacuo, and the resulting residue was purified by preparative HPLC (column: XBridge Prep OBD C18 Column, 30×150 mm, 5 μm; mobile phase A: water (10 mM NH₄HCO₃)+0.05% NH₃—H₂O, mobile phase B: ACN; flow rate: 60 mL/min; gradient: 24% B to 51% B in 7 min; wavelength: 220 nm & 254 nm; RT1 (min): 7.33). The desired fractions were combined, and then lyophilized to afford N-(5-chloro-4-(4-fluoro-1-isopropyl-2-methyl-1H-benzo[d]imidazol-6-yl)pyrimidin-2-yl)-4-((4-ethylpiperazin-1-yl)methyl)-1H-indazol-7-amine (22.7 mg, 19.0%, 96.4% purity) as a white amorphous solid. by LC/MS: RT=1.118 min, m/z [M+H]⁺=562.20.

### Example 28

### 7-(2,5-dichloropyrimidin-4-yl)-1-isopropyl-3,4-dihydroquinolin-2 (1H)-one (Synthetic Intermediate)

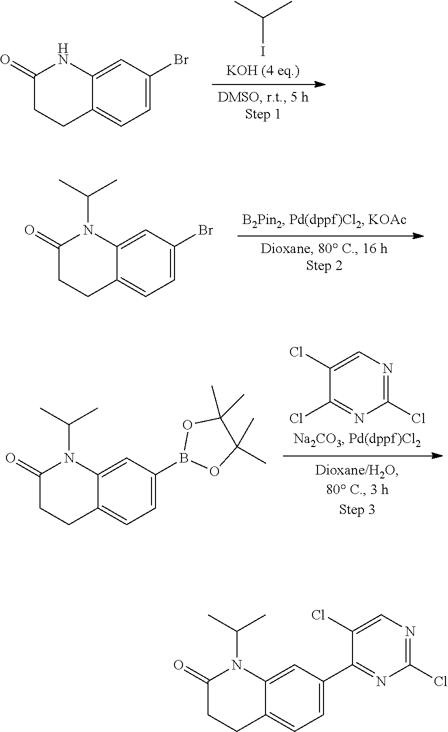

### 7-bromo-1-isopropyl-3,4-dihydroquinolin-2 (1H)-one

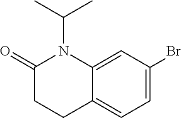

Step 1. A round-bottomed flask was charged with 7-bromo-3,4-dihydroquinolin-2 (1H)-one (2.5 g, 1 eq., 11 mmol), DMSO (5 mL), 2-iodopropane (5.6 g, 3 eq., 33 mmol), KOH (2.46 g, 4 eq., 44 mmol) and a stir bar, and the resulting solution was stirred at 25° C. for 5 hours. The reaction mixture was diluted with water (60 mL), the aq. phase was extracted with EtOAc (3×15 mL), and the combined organic layers were washed with brine, dried over sodium sulfate, filtered, and then concentrated in vacuo. The resulting residue was purified by reverse-phase flash chromatography (column: C18 silica gel; mobile phase: ACN in water; gradient: 30% to 60% in 45 min; wavelength: 254 nm). The desired fractions were combined, and then concentrated in vacuo to afford 7-bromo-1-isopropyl-3,4-dihydroquinolin-2 (1H)-one (1.5 g, 5.6 mmol, 51%) as a yellow oil. m/z (ES⁺) [M+H]⁺=268.05; HPLC tR=1.068 min.

### 1-isopropyl-7-(4,4,5,5-tetramethyl-1,3,2-dioxaborolan-2-yl)-3,4-dihydroquinolin-2 (1H)-one

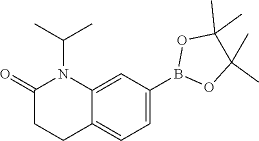 [SMILES: CC(C)N1C(=O)CCc2ccc(B3OC(C)(C)C(C)(C)O3)cc21]

Step 2. A resealable reaction vial was charged with 7-bromo-1-isopropyl-3,4-dihydroquinolin-2 (1H)-one (1.49 g, 1 eq., 5.56 mmol), 1,4-dioxane (5 mL), B₂Pin₂ (2.12 g, 1.5 eq., 8.33 mmol), KOAc (1.64 g, 3 eq., 16.7 mmol), Pd(dppf)Cl₂ (454 mg, 0.1 eq., 556 μmol) and a stir bar, and the resulting solution was evacuated, and then purged with nitrogen three times. The mixture was stirred at 80° C. for 16 hours, and then concentrated in vacuo. The resulting residue was diluted with water (10 mL), the aq. phase was extracted with EtOAc (3×10 mL), and the combined organic layers were washed with brine, dried over sodium sulfate, filtered, and then concentrated in vacuo to afford 1-isopropyl-7-(4,4,5,5-tetramethyl-1,3,2-dioxaborolan-2-yl)-3,4-dihydroquinolin-2 (1H)-one (1.51 g, 4.2 mmol, 75%, 87% purity) as a brown amorphous solid. m/z (ES⁺) [M+H]⁺=316.20; HPLC tR=1.135 min.

### 7-(2,5-dichloropyrimidin-4-yl)-1-isopropyl-3,4-dihydroquinolin-2 (1H)-one

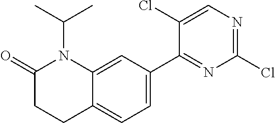 [SMILES: CC(C)N1C(=O)CCc2ccc(-c3nc(Cl)ncc3Cl)cc21]

Step 3. A resealable reaction vial was charged with 1-isopropyl-7-(4,4,5,5-tetramethyl-1,3,2-dioxaborolan-2-yl)-3,4-dihydroquinolin-2 (1H)-one (87% purity) (500 mg, 1 eq., 1.59 mmol), 1,4-dioxane/H₂O (3 mL), 2,4,5-trichloropyrimidine (407 mg, 1.4 eq., 2.22 mmol), Na₂CO₃ (168 mg, 1 eq., 1.59 mmol), Pd(dppf)Cl₂ (1.30 g, 1 eq., 1.59 mmol), and a stir bar, and the resulting mixture was evacuated, and then purged with nitrogen three times. The mixture was stirred at 80° C. for 3 hours, and then concentrated in vacuo. The resulting residue was diluted with water (2 mL), the aq. phase was extracted with EtOAc (3×5 mL), and the combined organic layers were washed with brine, dried over sodium sulfate, filtered, and then concentrated in vacuo. The resulting residue was purified by reverse-phase flash chromatography (column: C18 silica gel; mobile phase: ACN in water; gradient: 30% to 70% in 45 min; wavelength: 254 nm). The desired fractions were combined, and then concentrated in vacuo to afford 7-(2,5-dichloropyrimidin-4-yl)-1-isopropyl-3,4-dihydroquinolin-2 (1H)-one (340 mg, 1.01 mmol, 63.8%) as a yellow amorphous solid. m/z (ES⁺) [M+H]⁺=336.10; HPLC tR=1.086 min.

### Example 29

### 4-(3-(2,5-dichloropyrimidin-4-yl)pyrazolo[1,5-a]pyridin-6-yl)morpholine (Synthetic Intermediate)

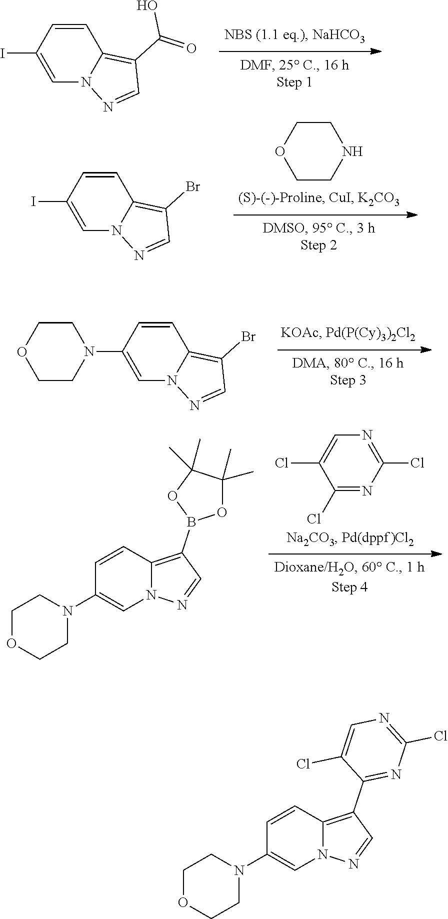

### 3-bromo-6-iodopyrazolo[1,5-a]pyridine

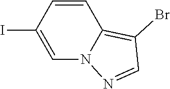 [SMILES: Brc1cnn2cc(I)ccc12]

Step 1. A solution of 6-iodopyrazolo[1,5-a]pyridine-3-carboxylic acid (990 mg, 1 eq., 3.44 mmol), N-bromosuccinimide (673 mg, 321 μL, 1.1 eq., 3.78 mmol) and sodium bicarbonate (722 mg, 334 μL, 2.5 eq., 8.59 mmol) in DMF (25 mL) was stirred at r.t. overnight, with monitoring by LC/MS. The reaction mixture was quenched with water, the aq. phase was extracted with EtOAc, and the organic layer was washed with brine, dried over Na₂SO₄, and concentrated in vacuo. The resulting residue was dissolved in DMF, and then purified by flash chromatography (column: C18; mobile phase A: water; mobile phase B: ACN; flow rate: 60 mL/min; gradient: 0% B to 100% B in 40 min; wavelength: 220 nm & 254 nm). The desired fractions were combined, and then concentrated in vacuo to afford 3-bromo-6-iodopyrazolo[1,5-a]pyridine (1 g, 2 mmol, 70%, 80% purity) as a yellow solid. LC/MS: RT=0.848 min, m/z [M+H]⁺=322.80.

### 4-(3-bromopyrazolo[1,5-a]pyridin-6-yl)morpholine

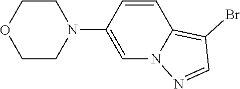 [SMILES: Brc1cnn2cc(N3CCOCC3)ccc12]

Step 2. To a solution of 3-bromo-6-iodopyrazolo[1,5-a]pyridine (600 mg, 1 eq., 1.86 mmol), morpholine (243 mg, 1.5 eq., 2.79 mmol) and (S)-(−)-proline (42.8 mg, 31.7 μL, 0.2 eq., 372 mol) in DMSO (15 mL) were added K₂CO₃ (514 mg, 2 eq., 3.72 mmol) and CuI (35.4 mg, 0.1 eq., 186 mol), and the resulting mixture was stirred at 95° C. under a nitrogen atmosphere for 3 hours. Next, the reaction mixture was diluted with water (60 mL), the aq. phase was extracted with EtOAc (100 mL), and the combined organic layers were washed with brine, dried over sodium sulfate, filtered, and then concentrated in vacuo. The resulting residue was purified by flash chromatography (column: C18; mobile phase A: water; mobile phase B: ACN; gradient: 0% B to 100% B in 20 min; wavelength: 254 nm). The desired fractions were combined, and then concentrated in vacuo to afford 4-(3-bromopyrazolo[1,5-a]pyridin-6-yl)morpholine (150 mg, 0.43 mmol, 23%, 80% purity) as a green solid. LC/MS: RT=1.048 min, m/z [M+H]⁺=281.95.

### 4-(3-(4,4,5,5-tetramethyl-1,3,2-dioxaborolan-2-yl)pyrazolo[1,5-a]pyridin-6-yl)morpholine

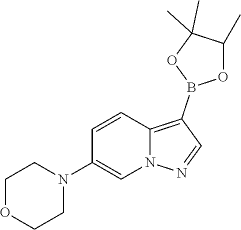 [SMILES: CC1OB(c2cnn3cc(N4CCOCC4)ccc23)OC1(C)C]

Step 3. To a stirring solution of 4-(3-bromopyrazolo[1,5-a]pyridin-6-yl)morpholine (150 mg, 1 eq., 532 mol), 4,4,4′,4′,5,5,5′,5′-octamethyl-2,2′-bi(1,3,2-dioxaborolane) (270 mg, 2 eq., 1.06 mmol) and potassium acetate (157 mg, 3 eq., 1.59 mmol) in DMA (5 mL) was added Pd(P(Cy)₃)₂Cl₂ (39.2 mg, 0.1 eq., 53.2 μmol), and the resulting solution was stirred at 80° C. under nitrogen atmosphere for 16 hours. The resulting mixture was filtered, the filter cake was washed with DCM, and the filtrate was concentrated in vacuo. The resulting residue was dissolved in DMF, and then purified by flash chromatography (column: C18, mobile phase A: water; mobile phase B: ACN; flow rate: 60 mL/min; gradient: 0% B to 100% B in 40 min; wavelength: 220 nm & 254 nm). The desired fractions were combined, and then concentrated in vacuo to afford 4-(3-(4,4,5,5-tetramethyl-1,3,2-dioxaborolan-2-yl)pyrazolo[1,5-a]pyridin-6-yl)morpholine (70 mg, 0.20 mmol, 37%, 92.7% Purity) as a yellow solid. LC/MS: RT=0.850 min, m/z [M+H]⁺=330.05.

### 4-(3-(2,5-dichloropyrimidin-4-yl)pyrazolo[1,5-a]pyridin-6-yl)morpholine

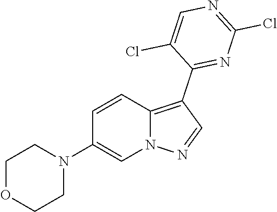 [SMILES: Clc1ncc(Cl)c(-c2cnn3cc(N4CCOCC4)ccc23)n1]

Step 4. To a stirring solution of 4-(3-(4,4,5,5-tetramethyl-1,3,2-dioxaborolan-2-yl)pyrazolo[1,5-a]pyridin-6-yl)morpholine (60 mg, 1 eq., 0.18 mmol), 2,4,5-trichloropyrimidine (50 mg, 1.5 eq., 0.27 mmol) and Na₂CO₃ (58 mg, 3 eq., 0.55 mmol) in 1,4-dioxane (3 mL) and water (0.8 mL) was added PdCl₂(dppf)-CH₂Cl₂ adduct (22 mg, 0.15 eq., 27 μmol), and the resulting solution was stirred at 60° C. under nitrogen atmosphere for 1 hour. Next, the reaction mixture was concentrated in vacuo. The resulting residue was dissolved in DMF, and then purified by flash chromatography (column: C18; mobile phase A: water; mobile phase B: ACN; flow rate: 60 mL/min; gradient: 0% B to 100% B in 40 min; wavelength: 220 nm & 254 nm). The desired fractions were combined, and then concentrated in vacuo to afford 4-(3-(2,5-dichloropyrimidin-4-yl)pyrazolo[1,5-a]pyridin-6-yl)morpholine (14 mg, 26 μmol, 15%, 66.2% purity) as a yellow solid. LC/MS: RT=0.875 min, m/z [M+H]⁺=349.95.

### Example 30

### 2-(5-chloro-2-(((3S,4R)-3-hydroxytetrahydro-2H-pyran-4-yl) amino) pyridin-4-yl)-7-cyclopropylpyrazolo[1,5-a]pyrazin-4 (5H)-one (Compound I-927)

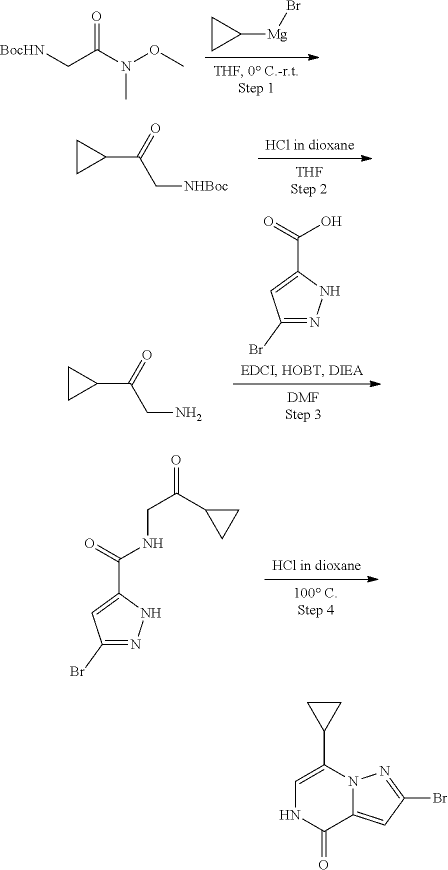

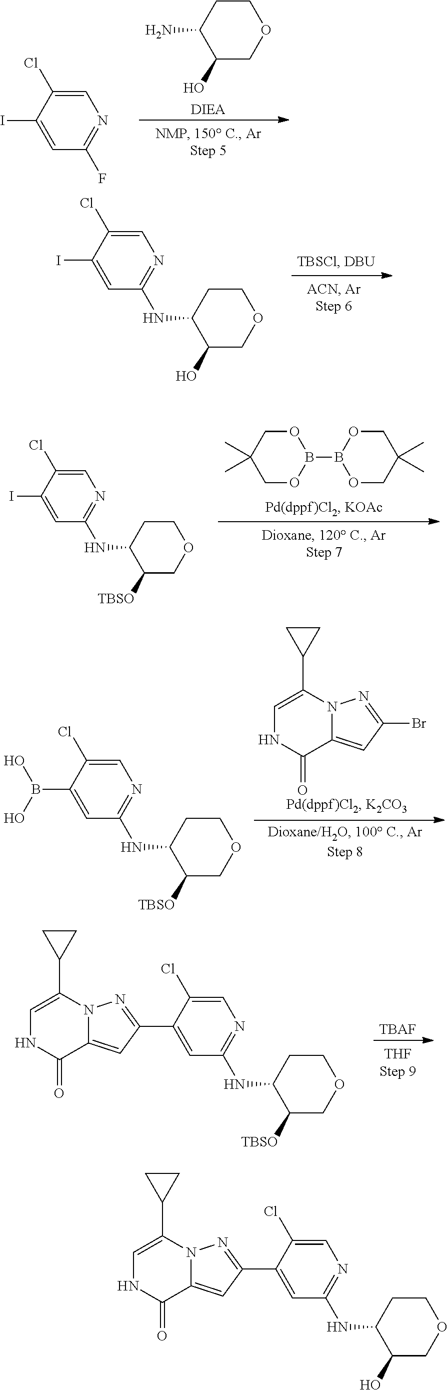

### tert-butyl (2-cyclopropyl-2-oxoethyl) carbamate

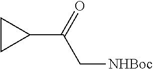 [SMILES: CC(C)(C)OC(=O)NCC(=O)C1CC1]

Step 1. A solution of tert-butyl (2-(methoxy(methyl)amino)-2-oxoethyl) carbamate (5 g, 1 eq., 0.02 mol) and cyclopropyl magnesium bromide (10 g, 70 mL, 1M, 3 eq., 0.07 mol) in THE (50 mL) was stirred at 25° C. for 16 hours. The reaction was quenched with sat'd. aq. NH₄Cl at 0° C., the aq. layer was extracted with EtOAc (3×100 mL), and the combined organic layers were concentrated in vacuo. The resulting residue was purified by silica-gel column chromatography, eluting with PE/EtOAc (1:1). The desired fractions were combined, and then concentrated in vacuo to afford tert-butyl (2-cyclopropyl-2-oxoethyl) carbamate (2 g, 0.01 mol, 40%) as a yellow oil. LC/MS: RT=0.933 min, m/z [M-Boc]⁺=100.25.

### 2-amino-1-cyclopropylethan-1-one

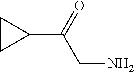

Step 2. A solution of tert-butyl (2-cyclopropyl-2-oxoethyl) carbamate (2 g, 1 eq., 0.01 mol) in a mixture of 4M HCl/dioxane (20 mL) and THE (20 mL) was stirred at 25° C. for 1 hour. The resulting mixture was concentrated in vacuo to afford crude 2-amino-1-cyclopropylethan-1-one (1.2 g, 12 mmol) as a yellow solid. LC/MS: RT=0.104 min, m/z [M+H]⁺=100.15.

### 3-bromo-N-(2-cyclopropyl-2-oxoethyl)-1H-pyrazole-5-carboxamide

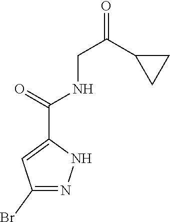 [SMILES: O=C(NCC(=O)C1CC1)c1cc(Br)n[nH]1]

Step 3. To a stirring solution of 2-amino-1-cyclopropylethan-1-one (1.2 g, 1 eq., 12 mmol) and 3-bromo-1H-pyrazole-5-carboxylic acid (3.5 g, 1.5 eq., 18 mmol) in DMF (12 mL) were added DIEA (16 g, 10 eq., 0.12 mol) and HATU (5.5 g, 1.2 eq., 15 mmol) in portions at 20° C. The resulting mixture was stirred at 25° C. for 1 hour. The reaction was quenched with water (30 mL) and the resulting mixture was extracted with EtOAc (3×30 mL). The combined organic extracts were washed with brine (3×30 mL), dried over anhydrous Na₂SO₄, and then concentrated in vacuo. The resulting residue was purified by preparative TLC, eluting with PE/EtOAc (1:1). The desired fractions were combined, and then concentrated in vacuo to afford 3-bromo-N-(2-cyclopropyl-2-oxoethyl)-1H-pyrazole-5-carboxamide (470 mg, 1.73 mmol, 14%) as a yellow solid. LC/MS: RT=0.421 min, m/z [M+H]⁺=271.95.

### 2-bromo-7-cyclopropylpyrazolo[1,5-a]pyrazin-4 (5H)-one

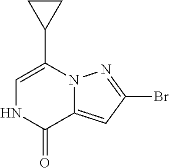 [SMILES: O=c1[nH]cc(C2CC2)n2nc(Br)cc12]

Step 4. A solution of 3-bromo-N-(2-cyclopropyl-2-oxoethyl)-1H-pyrazole-5-carboxamide (470 mg, 1 eq., 1.73 mmol) in HCl/dioxane (5 mL) was stirred at 100° C. for 12 hours. Next, the pH was adjusted to 7 with sat'd. (aq.) NaHCO₃, and the resulting mixture was extracted with EtOAc (3×5 mL). The combined organic layers were washed with brine (3×5 mL), dried over anhydrous Na₂SO₄, filtered, and then concentrated in vacuo to afford 2-bromo-7-cyclopropylpyrazolo[1,5-a]pyrazin-4 (5H)-one (420 mg, 1.65 mmol, 95.7%) as a brown solid. LC/MS: RT=0.603 min, m/z [M+H]⁺=254.00.

### (3S,4R)-4-((5-chloro-4-iodopyridin-2-yl) amino) tetrahydro-2H-pyran-3-ol

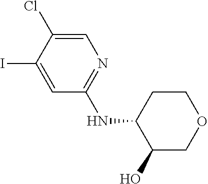 [SMILES: O[C@@H]1COCC[C@H]1Nc1cc(I)c(Cl)cn1]

Step 5. To a stirring solution of 5-chloro-2-fluoro-4-iodopyridine (2 g, 1 eq., 8 mmol) and (3S,4R)-4-aminotetrahydro-2H-pyran-3-ol (1 g, 1.2 eq., 9 mmol) in NMP (20 mL) was added DIEA (3 g, 3 eq., 0.02 mol) in portions at 25° C. under argon atmosphere, and the resulting mixture was stirred at 150° C. under argon atmosphere for 12 hours. Next, the resulting mixture was diluted with H₂O (100 mL), extracted with EtOAc (3×50 mL), and the combined organic layers were washed with sat'd. aq. NaCl (3×50 mL), dried over anhydrous Na₂SO₄, filtered, and then concentrated in vacuo. The resulting residue was purified by silica-gel column chromatography, eluting with PE/EtOAc (2:1). The desired fractions were combined, and then concentrated in vacuo to afford (3S,4R)-4-((5-chloro-4-iodopyridin-2-yl) amino) tetrahydro-2H-pyran-3-ol (2 g, 6 mmol, 70%) as a yellow solid. LC/MS: RT=0.749 min, m/z [M+H]⁺=354.9.

### N-((3S,4R)-3-((tert-butyldimethylsilyl) oxy) tetrahydro-2H-pyran-4-yl)-5-chloro-4-iodopyridin-2-amine

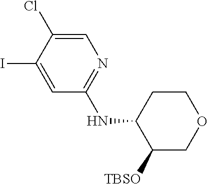 [SMILES: CC(C)(C)[Si](C)(C)O[C@@H]1COCC[C@H]1Nc1cc(I)c(Cl)cn1]

Step 6. To a stirring solution of (3S,4R)-4-((5-chloro-4-iodopyridin-2-yl) amino) tetrahydro-2H-pyran-3-ol (510 mg, 1 eq., 1.44 mmol) and DBU (657 mg, 3 eq., 4.32 mmol) in ACN (5 mL) was added TBSCl (542 mg, 2.5 eq., 3.60 mmol) in portions at 25° C. under argon atmosphere, and the resulting mixture was stirred at 20° C. under argon atmosphere for 12 hours. Next, the reaction mixture was diluted with H₂O (5 mL), the aq. phase was extracted with EtOAc (3×10 mL), and the combined organic layers were washed with sat'd. aq. NaCl (3×10 mL), dried over anhydrous Na₂SO₄, filtered, and then concentrated in vacuo. The resulting residue was purified by silica-gel chromatography, eluting with PE/EtOAc (9:1). The desired fractions were combined, and then concentrated in vacuo to afford N-((3S,4R)-3-((tert-butyldimethylsilyl) oxy) tetrahydro-2H-pyran-4-yl)-5-chloro-4-iodopyridin-2-amine (500 mg, 1.07 mmol, 74.1%) as a yellow solid. LC/MS: RT=1.599 min, m/z [M+H]⁺=469.55.

### (2-(((3S,4R)-3-((tert-butyldimethylsilyl) oxy) tetrahydro-2H-pyran-4-yl) amino)-5-chloropyridin-4-yl) boronic acid

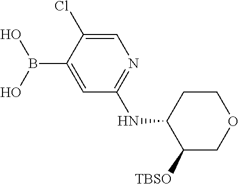 [SMILES: CC(C)(C)[Si](C)(C)O[C@@H]1COCC[C@H]1Nc1cc(B(O)O)c(Cl)cn1]

Step 7. To a stirring solution of N-((3S,4R)-3-((tert-butyldimethylsilyl) oxy) tetrahydro-2H-pyran-4-yl)-5-chloro-4-iodopyridin-2-amine (500 mg, 1 eq., 1.07 mmol) and KOAc (314 mg, 3 eq., 3.20 mmol) in dioxane (5 mL) were added Pd(dppf)Cl₂ (43.5 mg, 0.05 eq., 53.3 μmol) and 5,5,5′,5′-tetramethyl-2,2′-bi(1,3,2-dioxaborinane) (313 mg, 1.3 eq., 1.39 mmol) in portions at 25° C. under argon atmosphere, and the resulting mixture was stirred at 120° C. under argon atmosphere for 12 hours. Next, the reaction mixture was diluted with H₂O (10 mL), the aq. phase was extracted with EtOAc (3×10 mL), and the combined organic layers were washed with sat'd. aq. NaCl (3×10 mL), dried over anhydrous Na₂SO₄, filtered, and then concentrated in vacuo. The resulting residue was purified by silica-gel chromatography, eluting with DCM/MeOH (10:1). The desired fractions were combined, and then concentrated in vacuo to afford (2-(((3S,4R)-3-((tert-butyldimethylsilyl) oxy) tetrahydro-2H-pyran-4-yl) amino)-5-chloropyridin-4-yl) boronic acid (240 mg, 621 mol, 58.2%) as a yellow solid. LC/MS: RT=1.133 min, m/z [M+H]⁺=387.20.

### 2-(2-(((3S,4R)-3-((tert-butyldimethylsilyl) oxy) tetrahydro-2H-pyran-4-yl) amino)-5-chloropyridin-4-yl)-7-cyclopropylpyrazolo[1,5-a]pyrazin-4 (5H)-on

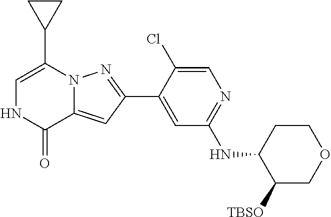 [SMILES: CC(C)(C)[Si](C)(C)O[C@@H]1COCC[C@H]1Nc1cc(-c2cc3c(=O)[nH]cc(C4CC4)n3n2)c(Cl)cn1]

Step 8. To a stirring solution of (2-(((3S,4R)-3-((tert-butyldimethylsilyl) oxy) tetrahydro-2H-pyran-4-yl) amino)-5-chloropyridin-4-yl) boronic acid (150 mg, 1 eq., 388 μmol) and K₂CO₃ (161 mg, 3 eq., 1.16 mmol) in dioxane (2 mL) and water (0.4 mL) were added Pd(dppf)Cl₂ (31.7 mg, 0.1 eq., 38.8 μmol) and 2-bromo-7-cyclopropylpyrazolo[1,5-a]pyrazin-4 (5H)-one (98.5 mg, 1 eq., 388 mol) in portions at 25° C. under argon atmosphere, and the resulting mixture was stirred at 100° C. under argon atmosphere for 2 hours. The resulting mixture was diluted with water (20 mL), the aq. phase was extracted with EtOAc (3×20 mL), and the combined organic layers were washed with brine (3×20 mL), dried over anhydrous Na₂SO₄, filtered, and then concentrated in vacuo. The resulting residue was purified by silica-gel chromatography, eluting with PE/EtOAc (1:1). The desired fractions were combined, and then concentrated in vacuo to afford 2-(2-(((3S,4R)-3-((tert-butyldimethylsilyl) oxy) tetrahydro-2H-pyran-4-yl) amino)-5-chloropyridin-4-yl)-7-cyclopropylpyrazolo[1,5-a]pyrazin-4 (5H)-one (100 mg, 0.15 mmol, 37%, 75% purity) as a yellow solid. LC/MS: RT=1.349, m/z [M+H]⁺=516.10.

### 2-(5-chloro-2-(((3S,4R)-3-hydroxytetrahydro-2H-pyran-4-yl) amino) pyridin-4-yl)-7-cyclopropylpyrazolo[1,5-a]pyrazin-4 (5H)-one

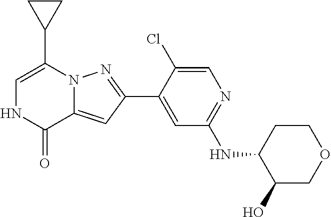 [SMILES: O=c1[nH]cc(C2CC2)n2nc(-c3cc(N[C@@H]4CCOC[C@H]4O)ncc3Cl)cc12]

Step 9. A solution of 2-(2-(((3S,4R)-3-((tert-butyldimethylsilyl) oxy) tetrahydro-2H-pyran-4-yl) amino)-5-chloropyridin-4-yl)-7-cyclopropylpyrazolo[1,5-a]pyrazin-4 (5H)-one (75 mg, 1 eq., 0.15 mmol) and TBAF (76 mg, 0.29 mL, 1M, 2 eq., 0.29 mmol) in THE (1 mL) was stirred at 20° C. for 2 hours. The resulting mixture was diluted with water (10 mL), the aq. phase was extracted with EtOAc (3×10 mL), and the combined organic layers were washed with brine (3×10 mL), dried over anhydrous Na₂SO₄, filtered, and then concentrated in vacuo. The resulting residue was purified by silica-gel chromatography, eluting with PE/EtOAc (1:1). The desired fractions were combined, and then concentrated in vacuo to afford crude 2-(5-chloro-2-(((3S,4R)-3-hydroxytetrahydro-2H-pyran-4-yl) amino) pyridin-4-yl)-7-cyclopropylpyrazolo[1,5-a]pyrazin-4 (5H)-one (50 mg) as a white solid. This crude product was purified by preparative HPLC (column: XBridge Shield RP18 OBD Column, 30×150 mm, 5 μm; mobile phase A: water (10 mM NH₄HCO₃+0.05% NH₃—H₂O); mobile phase B: ACN; flow rate: 60 mL/min; gradient: 21% B to 31% B in 10 min; wavelength: 220 nm & 254 nm; RT1 (min): 9.1). The desired fractions were combined, and then concentrated in vacuo to afford 2-(5-chloro-2-(((3S,4R)-3-hydroxytetrahydro-2H-pyran-4-yl) amino) pyridin-4-yl)-7-cyclopropylpyrazolo[1,5-a]pyrazin-4 (5H)-one (6.9 mg, 17 μmol, 12%) as a white solid.

### Example 31

### 2′-(2,5-dichloropyridin-4-yl)-5′,6′-dihydrospiro[cyclopropane-1,7′-pyrazolo[4,3-c]pyridin]-4′(2′H)-one (Synthetic Intermediate)

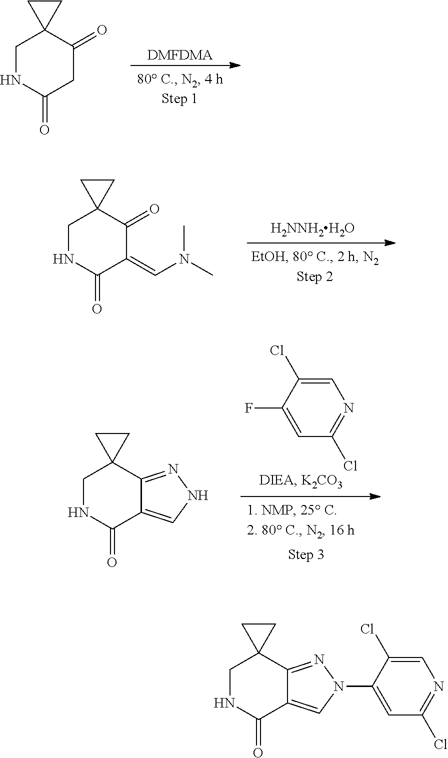

### (E)-7-((dimethylamino)methylene)-5-azaspiro[2.5]octane-6,8-dione

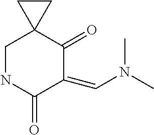

Step 1. A solution of 5-azaspiro [2.5]octane-6,8-dione (200 mg, 1 eq., 1.44 mmol) in DMFDMA (2 mL) was stirred at 80° C. under nitrogen atmosphere for 4 hours, with monitoring by LC/MS. Next, the resulting mixture was concentrated under in vacuo to afford crude (E)-7-((dimethylamino)methylene)-5-azaspiro[2.5]octane-6,8-dione (333 mg, 1.71 mmol) as a yellow oil. LC/MS: RT=0.480 min, m/z [M+H]⁺=195.10.

### 5′,6′-dihydrospiro[cyclopropane-1,7′-pyrazolo[4,3-c]pyridin]-4′(2′H)-one

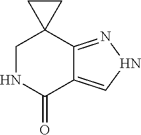 [SMILES: O=C1NCC2(CC2)c2n[nH]cc21]

Step 2. A mixture of (E)-7-((dimethylamino)methylene)-5-azaspiro [2.5]octane-6,8-dione (324 mg, 1 eq., 1.67 mmol) and hydrazine hydrate (104 mg, 102 μL, 80% wt., 1 eq., 1.67 mmol) in EtOH (1 mL) was stirred at 80° C. under nitrogen atmosphere for 2 hours, with monitoring by LC/MS. Next, the reaction mixture was concentrated in vacuo, and the resulting residue was purified by silica-gel chromatography, eluting with DCM/MeOH (10:1). The desired fractions were combined, and then concentrated in vacuo to afford 5′,6′-dihydrospiro[cyclopropane-1,7′-pyrazolo[4,3-c]pyridin]-4′(2′H)-one (200 mg, 1.23 mmol, 73.5%) as a white solid. LC/MS: RT=0.373 min, m/z [M+H]⁺=164.10.

### 2′-(2,5-dichloropyridin-4-yl)-5′,6′-dihydrospiro[cyclopropane-1,7′-pyrazolo[4,3-c]pyridin]-4′(2′H)-one

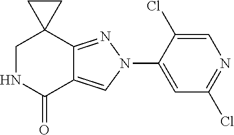 [SMILES: O=C1NCC2(CC2)c2nn(-c3cc(Cl)ncc3Cl)cc21]

Step 3. To a stirring mixture of 5′,6′-dihydrospiro[cyclopropane-1,7′-pyrazolo[4,3-c]pyridin]-4′(2′H)-one (100 mg, 1 eq., 613 mol) and 2,5-dichloro-4-fluoropyridine (112 mg, 1.1 eq., 674 μmol) in NMP (1 mL) were added DIEA (238 mg, 320 μL, 3 eq., 1.84 mmol) and K₂CO₃ (254 mg, 3 eq., 1.84 mmol) at 25° C., and the resulting mixture was stirred at 80° C. under nitrogen atmosphere for 16 hours, with monitoring by LC/MS. The resulting residue was purified by silica-gel chromatography, eluting with DCM/MeOH (10:1). The desired fractions were combined, and then concentrated in vacuo to afford 2′-(2,5-dichloropyridin-4-yl)-5′,6′-dihydrospiro[cyclopropane-1,7′-pyrazolo[4,3-c]pyridin]-4′(2′H)-one (200 mg, 647 μmol, crude) as a white solid. LC/MS: RT=1.000 min, m/z [M+H]⁺=309.00.

### Example 32

### (3S,4R)-4-((5-chloro-4-(1-methyl-5′,6′-dihydrospiro[piperidine-4,7′-pyrrolo[1,2-a]imidazol]-3′-yl)pyrimidin-2-yl)amino)tetrahydro-2H-pyran-3-ol formate (I-935)

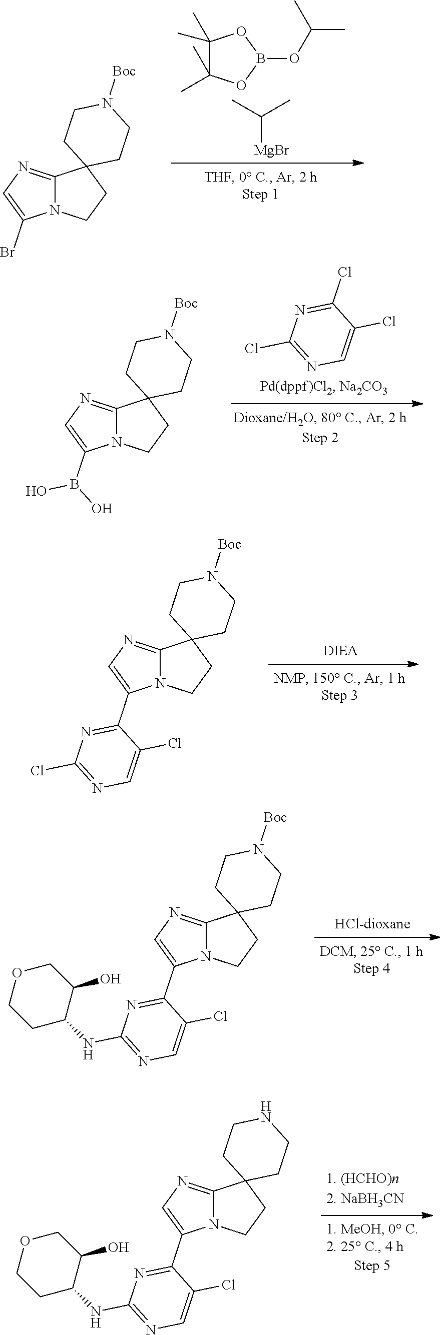

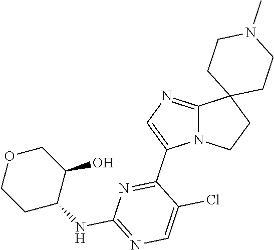 [SMILES: CN1CCC2(CC1)CCn1c(-c3nc(N[C@@H]4CCOC[C@H]4O)ncc3Cl)cnc12]

### (1-(tert-butoxycarbonyl)-5′,6′-dihydrospiro[piperidine-4,7′-pyrrolo[1,2-a]imidazol]-3′-yl)boronic acid

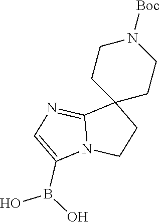 [SMILES: CC(C)(C)OC(=O)N1CCC2(CC1)CCn1c(B(O)O)cnc12]

Step 1. To a stirring solution of tert-butyl 3′-bromo-5′,6′-dihydrospiro[piperidine-4,7′-pyrrolo[1,2-a]imidazole]-1-carboxylate (400 mg, 1 eq., 1.12 mmol) and 2-isopropoxy-4,4,5,5-tetramethyl-1,3,2-dioxaborolane (627 mg, 3 eq., 3.37 mmol) in THF (1 mL) was added isopropylmagnesium bromide (827 mg, 4.32 mL, 1.3 molar, 5 eq., 5.61 mmol) in portions at 0° C. under argon atmosphere, and the resulting mixture was stirred at 0° C. under argon atmosphere for 2 hours. Next, the reaction mixture was diluted with aq. NH₄Cl (2 mL), and the aq. phase was extracted with EtOAc (3×5 mL). The combined organic layers were washed with brine (3×3 mL), dried over anhydrous Na₂SO₄, filtered, and then concentrated in vacuo to afford crude (1-(tert-butoxycarbonyl)-5′,6′-dihydrospiro[piperidine-4,7′-pyrrolo[1,2-a]imidazol]-3′-yl)boronic acid (470 mg, 1.25 mmol, crude) as a yellow solid. LC/MS: RT=0.622 min, m/z [M+H]⁺=322.1.

### tert-butyl 3′-(2,5-dichloropyrimidin-4-yl)-5′,6′-dihydrospiro[piperidine-4,7′-pyrrolo[1,2-a]imidazole]-1-carboxylate

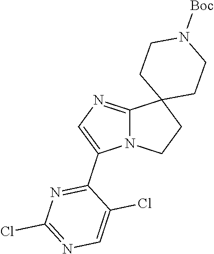 [SMILES: CC(C)(C)OC(=O)N1CCC2(CC1)CCn1c(-c3nc(Cl)ncc3Cl)cnc12]

Step 2. To a stirring solution of (1-(tert-butoxycarbonyl)-5′,6′-dihydrospiro[piperidine-4,7′-pyrrolo[1,2-a]imidazol]-3′-yl)boronic acid (590 mg, 1 eq., 1.84 mmol) and 2,4,5-trichloropyrimidine (337 mg, 1 eq., 1.84 mmol) in dioxane (6 mL) and water (0.6 mL) were added Pd(dppf)Cl₂ (150 mg, 0.1 eq., 184 μmol) and Na₂CO₃ (584 mg, 3 eq., 5.51 mmol) in portions at 25° C. under argon atmosphere, and the resulting mixture was stirred at 80° C. under argon atmosphere for 2 hours. Next, the reaction mixture was diluted with water (20 mL), the aq. phase was extracted with EtOAc (3×20 mL), and the combined organic layers were washed with brine (3×20 mL), dried over anhydrous Na₂SO₄, filtered, and then concentrated in vacuo. The resulting residue was purified by silica-gel chromatography, eluting with PE/EtOAc (1:1). The desired fractions were combined, and then concentrated in vacuo to afford tert-butyl 3′-(2,5-dichloropyrimidin-4-yl)-5′,6′-dihydrospiro[piperidine-4,7′-pyrrolo[1,2-a]imidazole]-1-carboxylate (310 mg, 731 μmol, 39.8%) as a yellow solid. LC/MS: RT=1.042 min, m/z [M+H]⁺=424.10.

### tert-butyl 3′-(5-chloro-2-(((3S,4R)-3-hydroxytetrahydro-2H-pyran-4-yl)amino)pyrimidin-4-yl)-5′,6′-dihydrospiro[piperidine-4,7′-pyrrolo[1,2-a]imidazole]-1-carboxylate

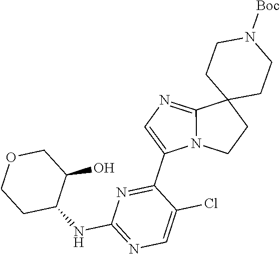 [SMILES: CC(C)(C)OC(=O)N1CCC2(CC1)CCn1c(-c3nc(N[C@@H]4CCOC[C@H]4O)ncc3Cl)cnc12]

Step 3. To a stirring solution of tert-butyl 3′-(2,5-dichloropyrimidin-4-yl)-5′,6′-dihydrospiro[piperidine-4,7′-pyrrolo[1,2-a]imidazole]-1-carboxylate (300 mg, 1 eq., 707 μmol) and (3S,4R)-4-aminotetrahydro-2H-pyran-3-ol (248 mg, 3 eq., 2.12 mmol) in NMP (3 mL) was added DIEA (274 mg, 3 eq., 2.12 mmol) in portions at 25° C. under argon atmosphere, and the resulting mixture was stirred at 150° C. under argon atmosphere for 1 hour, with monitoring by LC/MS. Next, the reaction mixture was diluted with water (10 mL), the aq. phase was extracted with EtOAc (3×10 mL), and the combined organic layers were washed with brine (3×10 mL), dried over anhydrous Na₂SO₄, filtered, and then concentrated in vacuo. The resulting residue was purified by silica-gel chromatography, eluting with PE/EtOAc (1:1). The desired fractions were combined, and then concentrated in vacuo to afford tert-butyl 3′-(5-chloro-2-(((3S,4R)-3-hydroxytetrahydro-2H-pyran-4-yl)amino)pyrimidin-4-yl)-5′,6′-dihydrospiro[piperidine-4,7′-pyrrolo[1,2-a]imidazole]-1-carboxylate (300 mg, 594 mol, 84.0%) as a yellow solid LC/MS: RT=0.742 min, m/z [M+H]⁺=505.25.

### (3S,4R)-4-((5-chloro-4-(5′,6′-dihydrospiro[piperidine-4,7′-pyrrolo[1,2-a]imidazol]-3′-yl)pyrimidin-2-yl)amino)tetrahydro-2H-pyran-3-ol

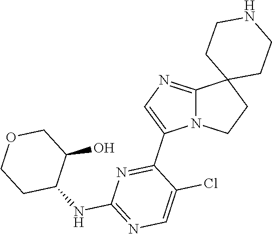 [SMILES: O[C@@H]1COCC[C@H]1Nc1ncc(Cl)c(-c2cnc3n2CCC32CCNCC2)n1]

Step 4. A solution of tert-butyl 3′-(5-chloro-2-(((3S,4R)-3-hydroxytetrahydro-2H-pyran-4-yl)amino)pyrimidin-4-yl)-5′,6′-dihydrospiro[piperidine-4,7′-pyrrolo[1,2-a]imidazole]-1-carboxylate (150 mg, 1 eq., 297 μmol) in HCl-dioxane (2 mL) and DCM (2 mL) was stirred at 25° C. for 1 hour. Next, the pH was brought to 8 with sat'd. aq. NaHCO₃, and the aq. phase was extracted with EtOAc (3×10 mL). The combined organic layers were washed with brine (3×10 mL), dried over anhydrous Na₂SO₄, filtered, and then concentrated in vacuo to afford (3S,4R)-4-((5-chloro-4-(5′,6′-dihydrospiro[piperidine-4,7′-pyrrolo[1,2-a]imidazol]-3′-yl)pyrimidin-2-yl)amino)tetrahydro-2H-pyran-3-ol (120 mg, 296 mol, crude) as a yellow solid. LC/MS: RT=0.543, m/z [M+H]⁺=405.10.

### (3S,4R)-4-((5-chloro-4-(1-methyl-5′,6′-dihydrospiro[piperidine-4,7′-pyrrolo[1,2-a]imidazol]-3′-yl)pyrimidin-2-yl)amino)tetrahydro-2H-pyran-3-ol formate

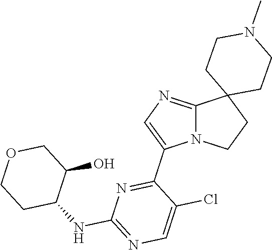 [SMILES: CN1CCC2(CC1)CCn1c(-c3nc(N[C@@H]4CCOC[C@H]4O)ncc3Cl)cnc12]

Step 5. To a stirring solution of (3S,4R)-4-((5-chloro-4-(5′,6′-dihydrospiro[piperidine-4,7′-pyrrolo[1,2-a]imidazol]-3′-yl)pyrimidin-2-yl)amino)tetrahydro-2H-pyran-3-ol (120 mg, 1 eq., 296 μmol) in MeOH (2 mL) was added paraformaldehyde (40.0 mg, 1.5 eq., 445 μmol) in portions at 0° C. The resulting mixture was stirred at 25° C. for 4 hours. Next, the resulting mixture was cooled to 0° C., NaBH₃CN (37.2 mg, 2 eq., 593 mol) was added in portions over 1 min, and the resulting mixture was then stirred at 25° C. for 2 hours. The crude product was purified by preparative HPLC (column: XSelect CSH Fluoro Phenyl, 30×150 mm, 5 μm; mobile phase A: water (0.1% formic acid); mobile phase B: MeOH; flow rate: 60 mL/min; gradient: 3% B to 20% B in 10 min, wavelength: 220 nm & 254 nm; RT1 (min): 8.14). The desired fractions were combined, and then concentrated in vacuo to afford (3S,4R)-4-((5-chloro-4-(1-methyl-5′,6′-dihydrospiro[piperidine-4,7′-pyrrolo[1,2-a]imidazol]-3′-yl)pyrimidin-2-yl)amino)tetrahydro-2H-pyran-3-ol formate (3.7 mg, 7.9 μmol, 2.7%, 99.5% purity) as a white solid. LC/MS: RT=0.683 min, m/z [M+H]⁺=419.15.

### Example 33

### (1R,3R,4R)-4-((5-chloro-4-(1-(1-methylpiperidin-4-yl)-1H-pyrazol-4-yl) pyrimidin-2-yl) amino)-3-hydroxycyclohexyl methylcarbamate (I-474)

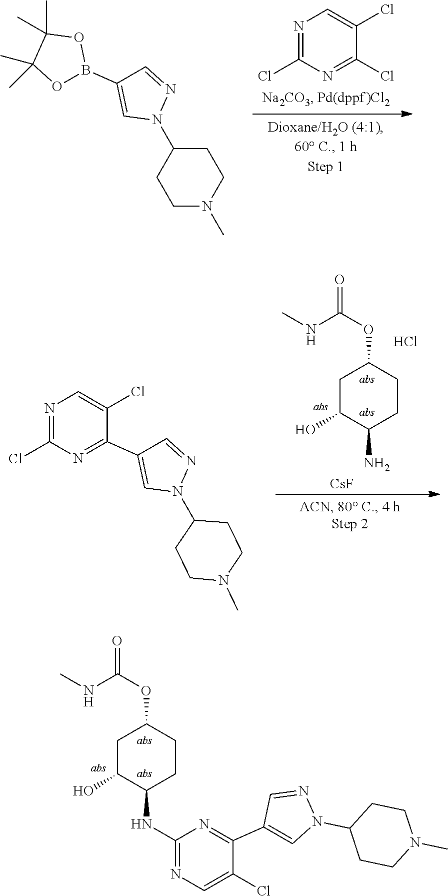

### 2,5-dichloro-4-(1-(1-methylpiperidin-4-yl)-1H-pyrazol-4-yl) pyrimidine

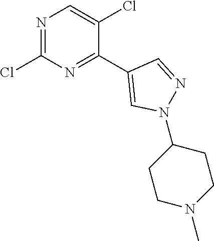 [SMILES: CN1CCC(n2cc(-c3nc(Cl)ncc3Cl)cn2)CC1]

Step 1. To a solution of 1-methyl-4-(4-(4,4,5,5-tetramethyl-1,3,2-dioxaborolan-2-yl)-1H-pyrazol-1-yl) piperidine (1 g, 1 eq., 3 mmol) in dioxane/H₂O (4:1, 10 mL) were added 2,4,5-trichloropyrimidine (0.8 g, 1.3 eq., 4 mmol) and Na₂CO₃ (1 g, 3 eq., 0.01 mol). Through the resulting reaction mixture was then bubbled nitrogen for 1 minute. Next, PdCl₂(dppf)-CH₂Cl₂ adduct (0.3 g, 0.1 eq., 0.3 mmol) was added, and the resulting reaction mixture was heated at 60° C. for 1 hour with vigorous stirring. The mixture was diluted with H₂O (10 mL), the aq. phase was extracted with EtOAc (3×20 mL), and the combined organic layers were washed with brine, dried over sodium sulfate, filtered, and then concentrated in vacuo. The crude residue was purified by flash chromatography (mobile phase A: water (10 mM NH₄HCO₃), mobile phase B: MeOH; flow rate: 90 mL/min; gradient: 100% B to 100% B in 10 min). The desired fractions were combined, and then concentrated in vacuo to afford 2,5-dichloro-4-(1-(1-methylpiperidin-4-yl)-1H-pyrazol-4-yl) pyrimidine (580 mg, 1.86 mmol, 50%) as a brown solid. LC/MS: RT=0.780 min, m/z [M+H]⁺=312.20.

### (1R,3R,4R)-4-((5-chloro-4-(1-(1-methylpiperidin-4-yl)-1H-pyrazol-4-yl) pyrimidin-2-yl) amino)-3-hydroxycyclohexyl methylcarbamate

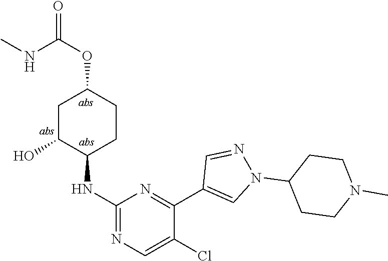

Step 2. A solution of 2,5-dichloro-4-(1-(1-methylpiperidin-4-yl)-1H-pyrazol-4-yl) pyrimidine (50 mg, 1 eq., 0.16 mmol), cesium fluoride (97 mg, 4 eq., 0.64 mmol) and (1R,3R,4R)-4-amino-3-hydroxycyclohexyl methylcarbamate hydrochloride (43 mg, 1.2 eq., 0.19 mmol; see Example 8) in acetonitrile (1.5 mL) was stirred at 80° C. for 4 hours. Next, the reaction mixture was concentrated in vacuo. The resulting residue was dissolved in DMSO, and then purified by preparative HPLC (column: XBridge Prep OBD C18 Column, 30×150 mm, 5 μm; mobile phase A: water (10 mM NH₄HCO₃)+0.05% NH₃·H₂O; mobile phase B: ACN; Flow rate: 60 mL/min; Gradient: 13% B to 40% B in 7 min; wavelength: 220 nm & 254 nm; RT1 (min): 7.63). The desired fractions were combined, and then lyophilized to afford (1R,3R,4R)-4-((5-chloro-4-(1-(1-methylpiperidin-4-yl)-1H-pyrazol-4-yl) pyrimidin-2-yl) amino)-3-hydroxycyclohexyl methylcarbamate (15.7 mg, 33.4 mol, 21%, 98.8% purity) as an off-white amorphous solid. LC/MS: RT=1.425 min, m/z [M+H]⁺=464.15.

### Example 34

### (3S,4R)-4-((10-(piperidin-4-yl)-6,7-dihydropyrazolo[1,5-d]pyrimido[4,5-f][1,4]oxazepin-2-yl)amino)tetrahydro-2H-pyran-3-ol (I-1052) & (3S,4R)-4-((10-(1-methylpiperidin-4-yl)-6,7-dihydropyrazolo[1,5-d]pyrimido[4,5-f][1,4]oxazepin-2-yl)amino)tetrahydro-2H-pyran-3-ol (I-1051)

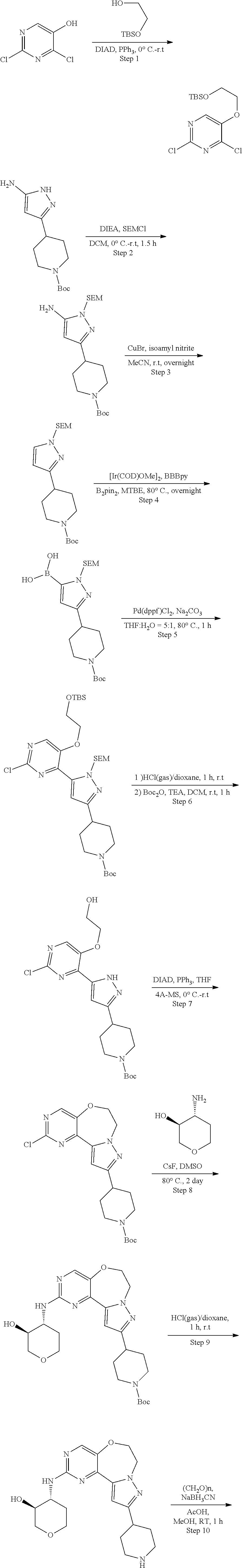

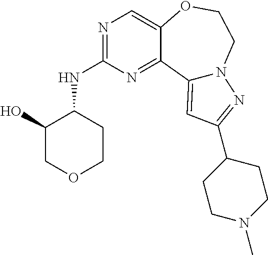 [SMILES: CN1CCC(c2cc3n(n2)CCOc2cnc(N[C@@H]4CCOC[C@H]4O)nc2-3)CC1]

### 5-(2-((tert-butyldimethylsilyl)oxy)ethoxy)-2,4-dichloropyrimidine

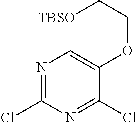 [SMILES: CC(C)(C)[Si](C)(C)OCCOc1cnc(Cl)nc1Cl]

Step 1. To a mixture of 2,4-dichloropyrimidin-5-ol (2 g, 0.01 mol) and 2-((tert-butyldimethylsilyl)oxy)ethan-1-ol (2 g, 10 mmol), Triphenylphosphine (6 g, 20 mmol) in THE (20 mL), DIAD (5 g, 20 mmol) was added dropwise at 0° C. under nitrogen atmosphere. The mixture was stirred for 25° C. at 2 hours. The resulting mixture was concentrated under vacuum. The residue was purified by reversed-phase flash chromatography with the following conditions: column, C18 silica gel; mobile phase, MeCN in Water, 30% to 90% gradient in 30 min; detector, UV 254 nm to afford 5-(2-((tert-butyldimethylsilyl)oxy)ethoxy)-2,4-dichloropyrimidine (2 g, 50%) as a white amorphous solid. m/z (ES⁺) [M+H]⁺=323.10; HPLC tR=1.030 min.

### tert-butyl 4-(5-amino-1-((2-(trimethylsilyl)ethoxy)methyl)-1H-pyrazol-3-yl)piperidine-1-carboxylate

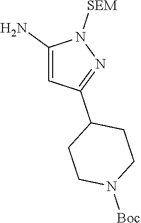 [SMILES: CC(C)(C)OC(=O)N1CCC(c2cc(N)n(COCC[Si](C)(C)C)n2)CC1]

Step 2. To a solution of tert-butyl 4-(5-amino-1H-pyrazol-3-yl)piperidine-1-carboxylate (9 g, 30 mmol) and DIEA (0.01 kg, 100 mmol) in DCM (100 mL) was added dropwise 2-(Chloromethoxyethyl)trimethyl silane (6 g, 40 mmol) at 0° C. under N₂ atmosphere. The reaction mixture was stirred at 25° C. for 1.5 hours. The reaction was quenched with water at rt. The resulting mixture was extracted with DCM (3×200 ml). The combined organic layers were washed with brine (1×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 10% to 90% gradient in 10 min; detector, UV 254 nm to afford tert-butyl 4-(5-amino-1-((2-(trimethylsilyl)ethoxy)methyl)-1H-pyrazol-3-yl)piperidine-1-carboxylate (6 g, 40%) as a yellow amorphous solid. m/z (ES⁺) [M+H]⁺=397.30; HPLC tR=0.830 min.

### tert-butyl 4-(1-((2-(trimethylsilyl)ethoxy)methyl)-1H-pyrazol-3-yl)piperidine-1-carboxylate

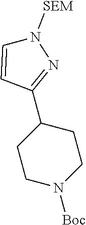 [SMILES: CC(C)(C)OC(=O)N1CCC(c2ccn(COCC[Si](C)(C)C)n2)CC1]

Step 3. A round bottom flask was charged with tert-butyl 4-(5-amino-1-((2-(trimethylsilyl)ethoxy)methyl)-1H-pyrazol-3-yl)piperidine-1-carboxylate (6 g, 10 mmol) and Isoamyl nitrite (3 g, 20 mmol), Cuprous bromide (2 g, 10 mmol) in MeCN (50 mL) and a stirbar, the solution was stirred for overnight at 20° C. The residue was purified by reversed-phase flash chromatography with the following conditions: column, C18 silica gel; mobile phase, MeCN in Water, 10% to 90% gradient in 10 min; detector, UV 254 nm to afford tert-butyl 4-(1-((2-(trimethylsilyl)ethoxy)methyl)-1H-pyrazol-3-yl)piperidine-1-carboxylate (1.5 g, 26%) a yellow solid. Compound 6: m/z (ES⁺) [M+H]⁺=382.25; HPLC tR=1.063 min.

### (3-(1-(tert-butoxycarbonyl)piperidin-4-yl)-1-((2-(trimethylsilyl)ethoxy)methyl)-1H-pyrazol-5-yl)boronic acid

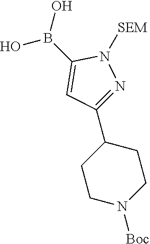 [SMILES: CC(C)(C)OC(=O)N1CCC(c2cc(B(O)O)n(COCC[Si](C)(C)C)n2)CC1]

Step 4. To a solution of tert-butyl 4-(1-((2-(trimethylsilyl)ethoxy)methyl)-1H-pyrazol-3-yl)piperidine-1-carboxylate (1.5 g, 5 mmol) and 4,4,4′,4′,5,5,5′,5′-octamethyl-2,2′-bi(1,3,2-dioxaborolane) (3 g, 10 mmol) in tert-Butyl methyl ether (20 mL), 4,4′-di-tert-butyl-2,2′-bipyridine (0.1 g, 0.5 mmol) and Dimethyliridium(1+) methanolate-(1Z,5Z)-1,5-cyclooctadiene (0.3 g, 0.5 mmol) were added. After stirring for 14 hours at 80° C. under a nitrogen atmosphere, the resulting mixture was filtered, the filter cake was washed with DCM (1×1 200 mL). The filtrate was concentrated under reduced pressure. The residue was purified by reversed-phase flash chromatography with the following conditions: column, C18 silica gel; mobile phase, MeCN in Water, 10% to 90% gradient in 10 min; detector, UV 254 nm to afford (3-(1-(tert-butoxycarbonyl)piperidin-4-yl)-1-((2-(trimethylsilyl)ethoxy)methyl)-1H-pyrazol-5-yl)boronic acid (1.5 g, 60%) as a brown amorphous oil. m/z (ES⁺) [M+H]⁺=426.30; HPLC tR=0.988 min.

### tert-butyl 4-(5-(5-(2-((tert-butyldimethylsilyl)oxy)ethoxy)-2-chloropyrimidin-4-yl)-1-((2-(trimethylsilyl)ethoxy)methyl)-1H-pyrazol-3-yl)piperidine-1-carboxylate

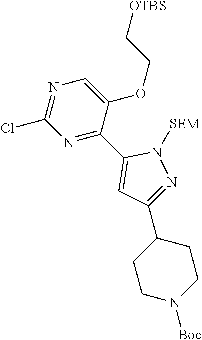 [SMILES: CC(C)(C)OC(=O)N1CCC(c2cc(-c3nc(Cl)ncc3OCCO[Si](C)(C)C(C)(C)C)n(COCC[Si](C)(C)C)n2)CC1]

Step 5. To a solution of 5-(2-((tert-butyldimethylsilyl)oxy)ethoxy)-2,4-dichloropyrimidine (1.5 g, 5.6 mmol) and (3-(1-(tert-butoxycarbonyl)piperidin-4-yl)-1-((2-(trimethylsilyl)ethoxy)methyl)-1H-pyrazol-5-yl)boronic acid (1.2 g, 2.8 mmol) in THF (50 mL)/Water (10 mL), Na₂CO₃ (0.90 g, 8.5 mmol) and Bis-(triphenylphosphino)-palladous chloride (0.20 g, 0.28 mmol) were added. After stirring for 80° C. at 1 hour under a nitrogen atmosphere. The resulting mixture was filtered, the filter cake was washed with DCM (1×1 300 ml). The filtrate was concentrated under reduced pressure. The residue was purified by reversed-phase flash chromatography with the following conditions: column, C18 silica gel; mobile phase, MeCN in Water, 10% to 50% gradient in 10 min; detector, UV 254 nm to afford tert-butyl 4-(5-(5-(2-((tert-butyldimethylsilyl)oxy)ethoxy)-2-chloropyrimidin-4-yl)-1-((2-(trimethylsilyl)ethoxy)methyl)-1H-pyrazol-3-yl)piperidine-1-carboxylate (990 mg, 47%) as a white amorphous solid. m/z (ES⁺) [M+H]⁺=668.50; HPLC tR=1.288 min.

### 4-(5-(2-chloro-5-(2-hydroxyethoxy)pyrimidin-4-yl)-1H-pyrazol-3-yl)piperidine-1-carboxylate

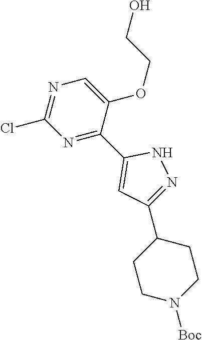 [SMILES: CC(C)(C)OC(=O)N1CCC(c2cc(-c3nc(Cl)ncc3OCCO)[nH]n2)CC1]

Step 6. A first round bottom flask was charged with tert-butyl 4-(5-(5-(2-((tert-butyldimethylsilyl)oxy)ethoxy)-2-chloropyrimidin-4-yl)-1-((2-(trimethylsilyl)ethoxy)methyl)-1H-pyrazol-3-yl)piperidine-1-carboxylate (990 mg, 1.48 mmol) and hydrogen chloride (20 ml, 4 molar, 80 mmol) in 1,4-Dioxane (100 mL) and a stirbar, the solution was stirred for 1 hour at 20° C. The resulting mixture was concentrated under vacuum to afford crude 2-((2-chloro-4-(3-(piperidin-4-yl)-1H-pyrazol-5-yl)pyrimidin-5-yl)oxy)ethan-1-ol (480 mg, 100%) as a yellow amorphous solid. m/z (ES⁺) [M+H]⁺=324.00; HPLC tR=0.506 min.

A second round bottom flask was charged with 2-((2-chloro-4-(3-(piperidin-4-yl)-1H-pyrazol-5-yl)pyrimidin-5-yl)oxy)ethan-1-ol (480 mg, 1.48 mmol) and TEA (3.00 g, 29.6 mmol), Di-tert-butyl dicarbonate (485 mg, 2.22 mmol) in DCM (100 mL) and a stirbar, the solution was stirred for 1 hour at 20° C. The resulting mixture was concentrated under vacuum. The residue was purified by reversed-phase flash chromatography with the following conditions: column, C18 silica gel; mobile phase, MeCN in Water, 10% to 100% gradient in 10 min; detector, UV 254 nm to afford tert-butyl 4-(5-(2-chloro-5-(2-hydroxyethoxy)pyrimidin-4-yl)-1H-pyrazol-3-yl)piperidine-1-carboxylate (250 mg, 36%) as a white amorphous solid. m/z (ES⁺) [M+H]⁺=424.20; HPLC tR=0.863 min.

### tert-butyl 4-(2-chloro-6,7-dihydropyrazolo[1,5-d]pyrimido[4,5-f][1,4]oxazepin-10-yl)piperidine-1-carboxylate

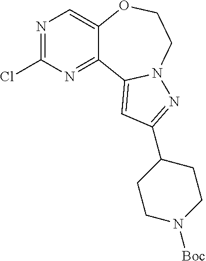 [SMILES: CC(C)(C)OC(=O)N1CCC(c2cc3n(n2)CCOc2cnc(Cl)nc2-3)CC1]

Step 7. A mixture of tert-butyl 4-(5-(2-chloro-5-(2-hydroxyethoxy)pyrimidin-4-yl)-1H-pyrazol-3-yl)piperidine-1-carboxylate (440 mg, 1.04 mmol) and Triphenylphosphine (545 mg, 2.08 mmol) and 4A-MS in THE (10 mL) under nitrogen atmosphere was stirred for 10 min at 0° C. Then to the above mixture DIAD (420 mg, 2.08 mmol) in THF (2 mL) was added into, and the resulting reaction was stirred for another 2 hours at r.t. The filtrate was concentrated under reduced pressure. The residue was purified by reversed-phase flash chromatography with the following conditions: column, C18 silica gel; mobile phase, MeCN in Water, 10% to 90% gradient in 10 min; detector, UV 254 nm to afford tert-butyl 4-(2-chloro-6,7-dihydropyrazolo[1,5-d]pyrimido[4,5-f][1,4]oxazepin-10-yl)piperidine-1-carboxylate (310 mg, 66%) as a white amorphous solid. m/z (ES⁺) [M+H]⁺=406.05; HPLC tR=0.870 min.

### tert-butyl 4-(2-(((3S,4R)-3-hydroxytetrahydro-2H-pyran-4-yl)amino)-6,7-dihydropyrazolo [1,5-d]pyrimido[4,5-f][1,4]oxazepin-10-yl)piperidine-1-carboxylate

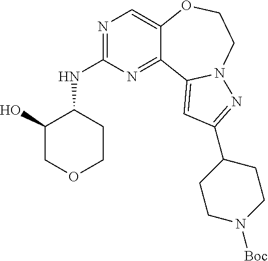 [SMILES: CC(C)(C)OC(=O)N1CCC(c2cc3n(n2)CCOc2cnc(N[C@@H]4CCOC[C@H]4O)nc2-3)CC1]

Step 8. A round bottom flask was charged with tert-butyl 4-(2-chloro-6,7-dihydropyrazolo[1,5-d]pyrimido[4,5-f][1,4]oxazepin-10-yl)piperidine-1-carboxylate (310 mg, 0.764 mmol) and (3S,4R)-4-aminotetrahydro-2H-pyran-3-ol (179 mg, 1.53 mmol), CsF (580 mg, 3.82 mmol) in DMSO (1.5 mL) and a stirbar, the solution was stirred for 2 day at 80° C. The resulting mixture was concentrated under vacuum. The residue was purified by reversed-phase flash chromatography with the following conditions: column, C18 silica gel; mobile phase, MeCN in Water, 10% to 90% gradient in 10 min; detector, UV 254 nm to afford tert-butyl 4-(2-(((3 S,4R)-3-hydroxytetrahydro-2H-pyran-4-yl)amino)-6,7-dihydropyrazolo [1,5-d]pyrimido[4,5-f][1,4]oxazepin-10-yl)piperidine-1-carboxylate (220 mg, 56%) as a colorless oil. m/z (ES⁺) [M+H]⁻=487.25; HPLC tR=0.898 min.

### (3S,4R)-4-((10-(piperidin-4-yl)-6,7-dihydropyrazolo[1,5-d]pyrimido[4,5-f][1,4]oxazepin-2-yl)amino)tetrahydro-2H-pyran-3-ol

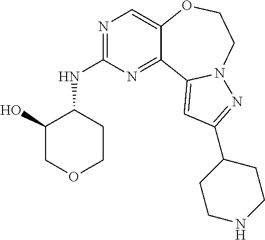 [SMILES: O[C@@H]1COCC[C@H]1Nc1ncc2c(n1)-c1cc(C3CCNCC3)nn1CCO2]

Step 9. A bottomed round flask was charged with tert-butyl 4-(2-(((3S,4R)-3-hydroxytetrahydro-2H-pyran-4-yl)amino)-6,7-dihydropyrazolo[1,5-d]pyrimido[4,5-f][1,4]oxazepin-10-yl)piperidine-1-carboxylate (100 mg, 0.206 mmol) and HCl (5 ml, 4 molar, 20 mmol) in 1,4-Dioxane (5 mL) and a stirbar, the solution was stirred for 1 hour at 20° C. The resulting mixture was concentrated under vacuum. The residue was purified by reverse flash chromatography with the following conditions: column, C18 silica gel; mobile phase, MeOH 100% gradient in 10 min; detector, UV 254 nm to afford crude (3S,4R)-4-((10-(piperidin-4-yl)-6,7-dihydropyrazolo[1,5-d]pyrimido[4,5-f][1,4]oxazepin-2-yl)amino)tetrahydro-2H-pyran-3-ol (38 mg, 48%). The crude (3S,4R)-4-((10-(piperidin-4-yl)-6,7-dihydropyrazolo[1,5-d]pyrimido[4,5-f][1,4]oxazepin-2-yl)amino)tetrahydro-2H-pyran-3-ol (8 mg, 0.020 mmol) was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 7% B to 25% B in 7 min; Wavelength: 254 nm/220 nm nm; RT1 (min): 6.55). Lyophilization yielded (3S,4R)-4-((10-(piperidin-4-yl)-6,7-dihydropyrazolo[1,5-d]pyrimido[4,5-f][1,4]oxazepin-2-yl)amino)tetrahydro-2H-pyran-3-ol (5.6 mg, 70.0%) as a white solid. m/z (ES⁺) [M+H]⁺=387.25; HPLC tR=3.879 min. ¹H NMR (400 MHz, DMSO-d₆) δ 8.17 (s, 1H), 6.82 (d, J=17.2 Hz, 2H), 4.96 (d, J=5.2 Hz, 1H), 4.62-4.56 (m, 2H), 4.41-4.35 (m, 2H), 3.95-3.60 (m, 3H), 3.56-3.36 (m, 2H), 3.18-2.92 (m, 3H), 2.87-2.52 (m, 3H), 1.89 (dd, J=69.6, 12.9 Hz, 3H), 1.54-1.37 (m, 3H).

### (3S,4R)-4-((10-(1-methylpiperidin-4-yl)-6,7-dihydropyrazolo[1,5-d]pyrimido[4,5-f][1,4]oxazepin-2-yl)amino)tetrahydro-2H-pyran-3-ol

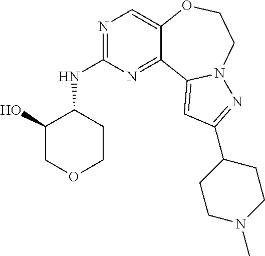 [SMILES: CN1CCC(c2cc3n(n2)CCOc2cnc(N[C@@H]4CCOC[C@H]4O)nc2-3)CC1]

Step 10. To a solution of (3S,4R)-4-((10-(piperidin-4-yl)-6,7-dihydropyrazolo[1,5-d]pyrimido[4,5-f][1,4]oxazepin-2-yl)amino)tetrahydro-2H-pyran-3-ol (70 mg, 0.18 mmol), Paraformaldehyde (54 mg, 1.8 mmol), Acetic acid (1.1 mg, 0.018 mmol) in MeOH (3 mL) was added NaCNBH₃ (0.11 g, 1.8 mmol) in portions at 0° C. under N₂ atmosphere. The reaction mixture was stirred at 0° C. for 2 min. The mixture was stirred for 2 hours at 20° C. The resulting mixture was concentrated under vacuum. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 10% B to 30% B in 7 min; Wavelength: 254 nm/220 nm nm; RT1 (min): 7.48). Lyophilization yielded (3S,4R)-4-((10-(1-methylpiperidin-4-yl)-6,7-dihydropyrazolo[1,5-d]pyrimido[4,5-f][1,4]oxazepin-2-yl)amino)tetrahydro-2H-pyran-3-ol (29.2 mg, 40%) as a white solid. m/z (ES⁺) [M+H]⁺=401.25; HPLC tR=1.056 min. ¹H NMR (400 MHz, DMSO-d₆) δ 8.18 (s, 1H), 6.90-6.77 (m, 2H), 4.96 (d, J=5.1 Hz, 1H), 4.62-4.55 (m, 2H), 4.45-4.28 (m, 2H), 3.89-3.67 (m, 3H), 3.49 (dq, J=9.3, 4.6 Hz, 1H), 3.39 (s, 1H), 3.07 (t, J=10.3 Hz, 1H), 2.80 (dd, J=9.1, 6.1 Hz, 2H), 2.55 (s, 1H), 2.17 (s, 3H), 1.95 (td, J=11.6, 2.4 Hz, 3H), 1.90-1.81 (m, 2H), 1.71-1.55 (m, 2H), 1.44 (qd, J=11.5, 4.5 Hz, 1H).

### Example 35

### (3S,4R)-4-((4-(6-(4-amino-4-methylpiperidin-1-yl)pyrazolo[1,5-a]pyridin-3-yl)-5-chloropyrimidin-2-yl)amino)tetrahydro-2H-pyran-3-ol (I-979)

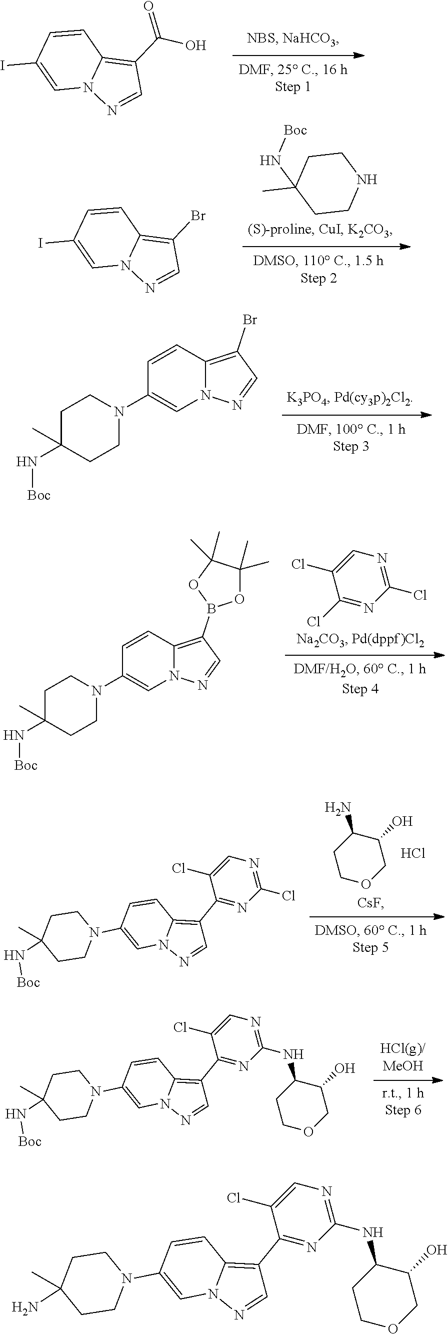

### 3-bromo-6-iodopyrazolo[1,5-a]pyridine

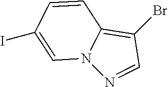 [SMILES: Brc1cnn2cc(I)ccc12]

Step 1. To a mixture of 6-iodopyrazolo[1,5-a]pyridine-3-carboxylic acid (3 g, 0.01 mol) in DMF (60 mL) was added sodium bicarbonate (2 g, 0.03 mol) and NBS (2 g, 0.01 mol) in portions at 25° C. The mixture was stirred for 16 hours at 25° C. The solution was purified by reverse flash chromatography with the following conditions: column, C18 silica gel; mobile phase, ACN in water, 10% to 100% gradient in 20 min; detector, UV 254 nm. Concentration in vacuo resulted in 3-bromo-6-iodopyrazolo[1,5-a]pyridine (1.8 g, 5.0 mmol, 50% yield, 90% purity) as a light yellow solid. m/z (ES⁺) [M+H]⁺=322.80; HPLC tR=0.847 min.

### tert-butyl (1-(3-bromopyrazolo[1,5-a]pyridin-6-yl)-4-methylpiperidin-4-yl)carbamate

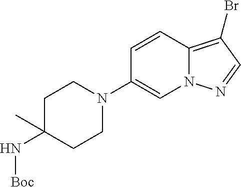 [SMILES: CC1(NC(=O)OC(C)(C)C)CCN(c2ccc3c(Br)cnn3c2)CC1]

Step 2. To a mixture of 3-bromo-6-iodopyrazolo[1,5-a]pyridine (1.8 g, 5.6 mmol), (S)-(−)-Proline (0.13 g, 0.11 mL, 1.1 mmol) and tert-butyl (4-methylpiperidin-4-yl)carbamate (1.8 g, 8.4 mmol) in DMSO (60 mL) was added CuI (0.11 g, 0.56 mmol) and K₂CO₃ (1.5 g, 11 mmol) in portions at 25° C. under nitrogen atmosphere. The mixture was stirred for 1.5 hours at 110° C. The mixture was cooled to 25° C. The reaction mixture was diluted with water (200 mL), and the aqueous phase was extracted with EA (300 mL) three times. The combined organic layers were washed with brine, dried over sodium sulfate, filtered, and concentrated in vacuo. The residue was purified by reverse flash chromatography with the following conditions: column, C18 silica gel; mobile phase, ACN in water, 10% to 100% gradient in 30 min; detector, UV 254 nm. Concentrated in vacuo resulted in tert-butyl (1-(3-bromopyrazolo[1,5-a]pyridin-6-yl)-4-methylpiperidin-4-yl)carbamate (1 g, 2 mmol, 40%, 90% Purity) as an off-white solid. m/z (ES⁺) [M+3H]⁺=411.05; HPLC tR=1.121 min.

### tert-butyl (4-methyl-1-(3-(4,4,5,5-tetramethyl-1,3,2-dioxaborolan-2-yl)pyrazolo[1,5-a]pyridin-6-yl)piperidin-4-yl)carbamate

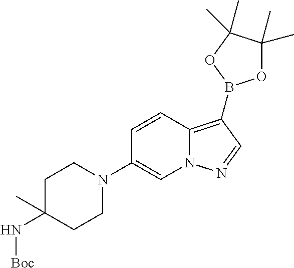 [SMILES: CC1(NC(=O)OC(C)(C)C)CCN(c2ccc3c(B4OC(C)(C)C(C)(C)O4)cnn3c2)CC1]

Step 3. A resealable reaction vial was charged with tert-butyl (1-(3-bromopyrazolo[1,5-a]pyridin-6-yl)-4-methylpiperidin-4-yl)carbamate (1 g, 2 mmol) in DMF (5 mL), Bis(pinacolato)diborane (2 g, 6 mmol), K₂CO₃ (1 g, 7 mmol), and a stirbar before being evacuated and purged with nitrogen three times. Dichloro(tricyclohexylphosphine)palladium (II) (0.3 g, 0.4 mmol) was added at 25° C. The resulting mixture was stirred at 100° C. for 1 hour under nitrogen atmosphere. The mixture was cooled to 25° C. The solution was purified by reverse flash chromatography with the following conditions: column, C18 silica gel; mobile phase, ACN in water, 10% to 100% gradient in 25 min; detector, UV 254 nm. Concentrated in vacuo resulted in (tert-butyl (4-methyl-1-(3-(4,4,5,5-tetramethyl-1,3,2-dioxaborolan-2-yl)pyrazolo[1,5-a]pyridin-6-yl)piperidin-4-yl)carbamate (308 mg, 675 μmol, 30%)) as an off-white solid. m/z (ES⁺) [M+H]⁺=457.20; HPLC tR=0.942 min.

### tert-butyl (1-(3-(2,5-dichloropyrimidin-4-yl)pyrazolo[1,5-a]pyridin-6-yl)-4-methylpiperidin-4-yl)carbamate

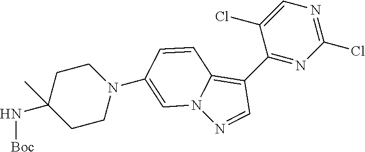 [SMILES: CC1(NC(=O)OC(C)(C)C)CCN(c2ccc3c(-c4nc(Cl)ncc4Cl)cnn3c2)CC1]

Step 4. A resealable reaction vial was charged with tert-butyl (4-methyl-1-(3-(4,4,5,5-tetramethyl-1,3,2-dioxaborolan-2-yl)pyrazolo[1,5-a]pyridin-6-yl)piperidin-4-yl)carbamate (240 mg, 526 mol) in DMF (5 mL) and Water (1 mL), 2,4,5-trichloropyrimidine (116 mg, 631 mol), Na₂CO₃ (167 mg, 1.58 mmol), and a stirbar before being evacuated and purged with nitrogen three times. Pd(dppf)Cl₂ (38.5 mg, 52.6 mol) was added at 25° C. The resulting mixture was stirred at 60° C. for 1 hour under nitrogen atmosphere. The mixture was cooled to 25° C. and purified by reverse flash chromatography with the following conditions: column, C18 silica gel; mobile phase, ACN in water, 10% to 100% gradient in 20 min; detector, UV 254 nm. Concentrated in vacuo resulted in (tert-butyl (1-(3-(2,5-dichloropyrimidin-4-yl)pyrazolo[1,5-a]pyridin-6-yl)-4-methylpiperidin-4-yl)carbamate (140 mg, 0.23 mmol, 45%, 80% Purity)) as an off-white solid. m/z (ES⁺) [M+H]⁺=477.10; HPLC tR=1.241 min.

### tert-butyl (1-(3-(5-chloro-2-(((3S,4R)-3-hydroxytetrahydro-2H-pyran-4-yl)amino)pyrimidin-4-yl)pyrazolo[1,5-a]pyridin-6-yl)-4-methylpiperidin-4-yl)carbamate

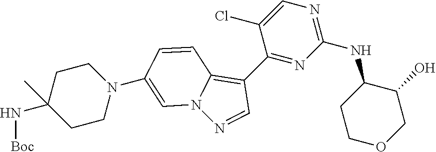 [SMILES: CC1(NC(=O)OC(C)(C)C)CCN(c2ccc3c(-c4nc(N[C@@H]5CCOC[C@H]5O)ncc4Cl)cnn3c2)CC1]

Step 5. A round bottom flask was charged with tert-butyl (1-(3-(2,5-dichloropyrimidin-4-yl)pyrazolo[1,5-a]pyridin-6-yl)-4-methylpiperidin-4-yl)carbamate (140 mg, 293 μmol), (3S,4R)-4-aminotetrahydro-2H-pyran-3-ol hydrochloride (67.6 mg, 440 μmol), CsF (223 mg, 1.47 mmol) and a stirbar. DMSO (3 mL) was added, and the solution was stirred for 1 hour at 60° C. The mixture was cooled to 25° C. and purified by reverse flash chromatography with the following conditions: column, C18 silica gel; mobile phase, ACN in water, 10% to 100% gradient in 20 min; detector, UV 254 nm. Concentrated in vacuo resulted in (tert-butyl (1-(3-(5-chloro-2-(((3S,4R)-3-hydroxytetrahydro-2H-pyran-4-yl)amino)pyrimidin-4-yl)pyrazolo[1,5-a]pyridin-6-yl)-4-methylpiperidin-4-yl)carbamate (80 mg, 0.14 mmol, 46%, 95% Purity)) as a white solid. m/z (ES⁺) [M+H]⁻=558.50; HPLC tR=0.738 min.

### (3S,4R)-4-((4-(6-(4-amino-4-methylpiperidin-1-yl)pyrazolo[1,5-a]pyridin-3-yl)-5-chloropyrimidin-2-yl)amino)tetrahydro-2H-pyran-3-ol

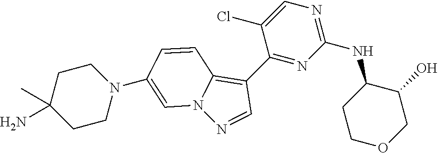 [SMILES: CC1(N)CCN(c2ccc3c(-c4nc(N[C@@H]5CCOC[C@H]5O)ncc4Cl)cnn3c2)CC1]

Step 6. A round bottomed flask was charged with tert-butyl (1-(3-(5-chloro-2-(((3S,4R)-3-hydroxytetrahydro-2H-pyran-4-yl)amino)pyrimidin-4-yl)pyrazolo[1,5-a]pyridin-6-yl)-4-methylpiperidin-4-yl)carbamate (75 mg, 0.13 mmol) and a stirbar. MeOH (1 mL) and 4M HCl in 1,4-diox. (1 mL) were added, and the solution was stirred for 1 hour at 25° C. The reaction mixture was concentrated in vacuo. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 13% B to 40% B in 7 min; Wavelength: 254 nm/220 nm nm; RT1 (min): 7.47). Lyophilization yielded (3S,4R)-4-((4-(6-(4-amino-4-methylpiperidin-1-yl)pyrazolo[1,5-a]pyridin-3-yl)-5-chloropyrimidin-2-yl)amino)tetrahydro-2H-pyran-3-ol (32.0 mg, 69.8 μmol, 52%, 99.9% Purity) as an off-white solid. m/z (ES⁺) [M+H]⁺=458.20; HPLC tR=0.818 min. ¹H NMR (400 MHz, DMSO-d6) 8.78 (s, 1H), 8.60 (s, 1H), 8.29-8.16 (m, 2H), 7.56-7.29 (s, 2H), 5.01 (d, J=5.2 Hz, 1H), 3.94-3.74 (m, 3H), 3.59-3.47 (m, 2H), 3.21 (t, J=5.5 Hz, 4H), 3.10 (s, 1H), 1.99 (s, 1H), 1.74 (s, 2H), 1.59-1.51 (dt, J=13.0, 4.8 Hz, 5H), 1.10 (s, 3H).

### Example 36

### (3R,4R)-4-((5-chloro-4-(2-(1-methylpiperidin-4-yl)oxazol-5-yl)pyrimidin-2-yl)amino)-1-(methylsulfonyl)piperidin-3-ol (I-972)

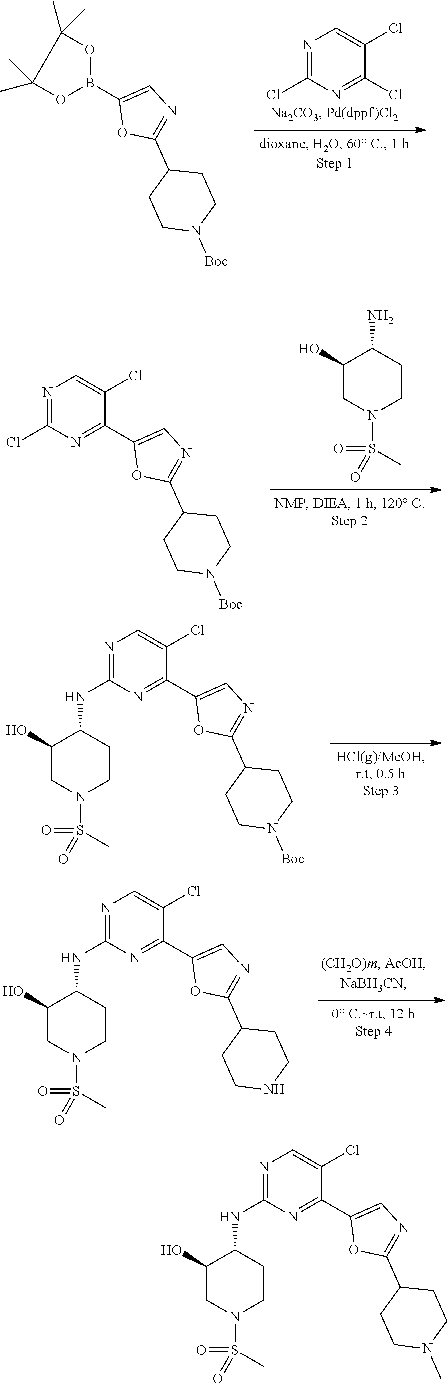

### tert-butyl 4-(5-(2,5-dichloropyrimidin-4-yl)oxazol-2-yl)piperidine-1-carboxylate

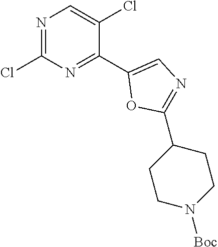 [SMILES: CC(C)(C)OC(=O)N1CCC(c2ncc(-c3nc(Cl)ncc3Cl)o2)CC1]

Step 1. A resealable reaction vial was charged with 2,4,5-trichloropyrimidine (800 mg, 4.36 mmol), tert-butyl 4-(5-(4,4,5,5-tetramethyl-1,3,2-dioxaborolan-2-yl)oxazol-2-yl)piperidine-1-carboxylate (2.47 g, 1.5 Eq, 6.54 mmol), Na₂CO₃ (1.39 g, 13.1 mmol), [1,1′-Bis(diphenylphosphino)ferrocene]dichloropalladium(II)ComplexWithDichloromethane (356 mg, 436 mol), 1,4-Dioxane (8 mL), H₂O (2 mL) and a stirbar before being evacuated and purged with nitrogen three times, and the mixture was stirred for 1 h at 60° C. The reaction mixture was concentrated and purified by Prep-TLC (PE/EA; ratio: 1/1) to afford tert-butyl 4-(5-(2,5-dichloropyrimidin-4-yl)oxazol-2-yl)piperidine-1-carboxylate (180 mg, 8.3%) as a yellow oil. m/z (ES⁺) [M+H]⁺=399.10; HPLC tR=0.873 min.

### tert-butyl 4-(5-(5-chloro-2-(((3R,4R)-3-hydroxy-1-(methylsulfonyl)piperidin-4-yl)amino)pyrimidin-4-yl)oxazol-2-yl)piperidine-1-carboxylate

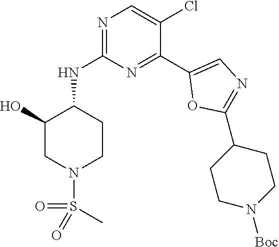 [SMILES: CC(C)(C)OC(=O)N1CCC(c2ncc(-c3nc(N[C@@H]4CCN(S(C)(=O)=O)C[C@H]4O)ncc3Cl)o2)CC1]

Step 2. A round bottom flask was charged with (3R,4R)-4-amino-1-(methylsulfonyl)piperidin-3-ol (99.2 mg, 511 μmol), tert-butyl 4-(5-(2,5-dichloropyrimidin-4-yl)oxazol-2-yl)piperidine-1-carboxylate (170 mg, 426 mol), DIEA (275 mg, 2.13 mmol), NMP (1 mL) and a stirbar before being evacuated and purged with nitrogen three times, and the solution was stirred for 1 h at 120° C. The reaction mixture was purified by reverse flash chromatography with the following conditions: column, C18 silica gel; mobile phase, MeCN in water, 0% to 100% gradient in 30 min; detector, UV 254 nm, Concentration in vacuo resulted in tert-butyl 4-(5-(5-chloro-2-(((3R,4R)-3-hydroxy-1-(methylsulfonyl)piperidin-4-yl)amino)pyrimidin-4-yl)oxazol-2-yl)piperidine-1-carboxylate (80 mg, 34%) as a colorless oil. m/z (ES⁺) [M+H]⁺=557.25; HPLC tR=0.846 min.

### (3R,4R)-4-((5-chloro-4-(2-(piperidin-4-yl)oxazol-5-yl)pyrimidin-2-yl)amino)-1-(methylsulfonyl)piperidin-3-ol

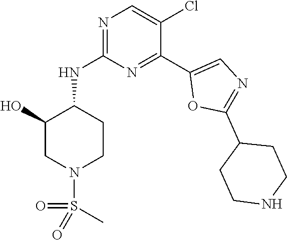 [SMILES: CS(=O)(=O)N1CC[C@@H](Nc2ncc(Cl)c(-c3cnc(C4CCNCC4)o3)n2)[C@H](O)C1]

Step 3. A round bottomed flask was charged with tert-butyl 4-(5-(5-chloro-2-(((3R,4R)-3-hydroxy-1-(methylsulfonyl)piperidin-4-yl)amino)pyrimidin-4-yl)oxazol-2-yl)piperidine-1-carboxylate (70 mg, 0.13 mmol), Hydrogen chloride (3.1 mL, 4 molar in dioxane), MeOH (3 mL) and a stirbar before being evacuated and purged with nitrogen three times, and the solution was stirred for 1 h at 25° C. The solution was concentrated. The mixture was quenched with saturated NaHCO₃(aq.). The solution was concentrated under vacuum to get the (3R,4R)-4-((5-chloro-4-(2-(piperidin-4-yl)oxazol-5-yl)pyrimidin-2-yl)amino)-1-(methylsulfonyl)piperidin-3-ol (150 mg, 91% purity, crude) as a white solid. The crude product was used in the next step directly without further purification. m/z (ES⁺) [M+H]⁺=457.20; HPLC tR=0.523 min.

### (3R,4R)-4-((5-chloro-4-(2-(1-methylpiperidin-4-yl)oxazol-5-yl)pyrimidin-2-yl)amino)-1-(methylsulfonyl)piperidin-3-ol

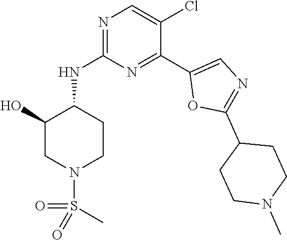 [SMILES: CN1CCC(c2ncc(-c3nc(N[C@@H]4CCN(S(C)(=O)=O)C[C@H]4O)ncc3Cl)o2)CC1]

Step 4. A resealable reaction vial was charged with (3R,4R)-4-((5-chloro-4-(2-(piperidin-4-yl)oxazol-5-yl)pyrimidin-2-yl)amino)-1-(methylsulfonyl)piperidin-3-ol (145 mg, 317 μmol), sodium cyanotrihydroborate (59.8 mg, 952 μmol), acetic acid (19.1 mg, 317 μmol), Paraformaldehyde (28.6 mg, 952 mol), MeOH (2 mL) and a stirbar before being evacuated and purged with nitrogen three times, and the mixture was stirred for 12 h at 25° C. The resulting crude material was purified by Pre-HPLC (Column: XBridge Prep OBD C18 Column, 30×150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 13% B to 38% B in 8 min; Wavelength: 220 nm nm; RT1 (min): 7.4). Lyophilization yielded (3R,4R)-4-((5-chloro-4-(2-(1-methylpiperidin-4-yl)oxazol-5-yl)pyrimidin-2-yl)amino)-1-(methylsulfonyl)piperidin-3-ol (7.3 mg, 4.9%) as a white solid. m/z (ES⁺) [M+H]⁺=471.15; HPLC tR=0.775 min. ¹H NMR (400 MHz, DMSO, 23° C.) δ 8.42 (s, 1H), 8.01 (s, 1H), 7.55 (s, 1H), 5.18-5.24 (m, 1H), 3.76 (s, 1H), 3.60 (d, J=10.0 Hz, 2H), 3.48 (d, J=12.2 Hz, 1H), 2.74-2.91 (m, 7H), 2.64 (s, 1H), 2.18 (s, 3H), 2.02 (d, J=11.3 Hz, 5H), 1.72-1.84 (m, 2H), 1.52 (d, J=11.5 Hz, 1H).

### Example 37

### (3S,4R)-4-((5-chloro-4-(2-(1-methylpiperidin-4-yl)oxazol-5-yl)pyrimidin-2-yl)amino)tetrahydro-2H-pyran-3-ol (I-931)

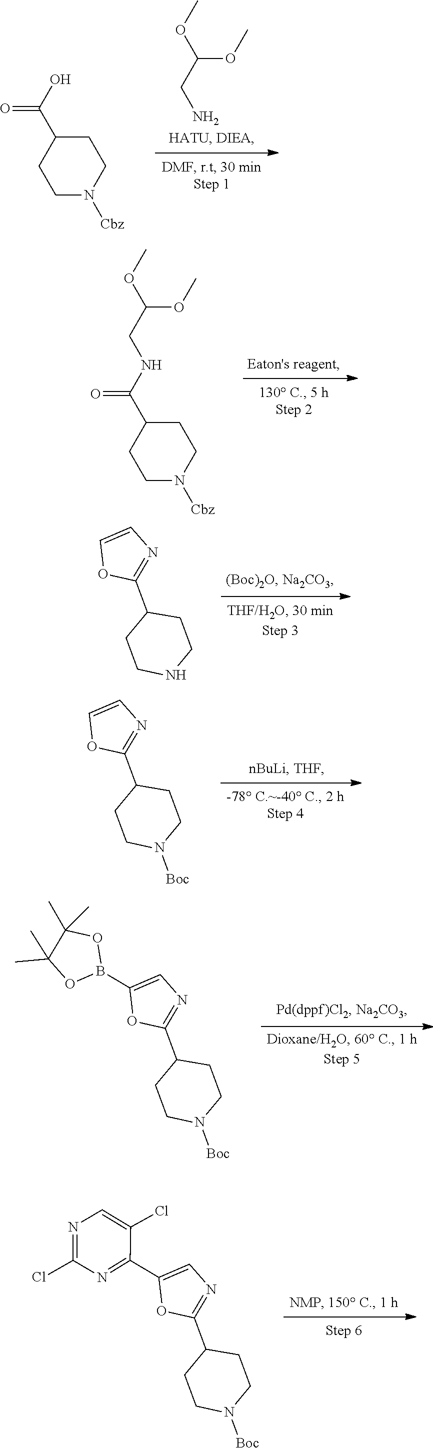

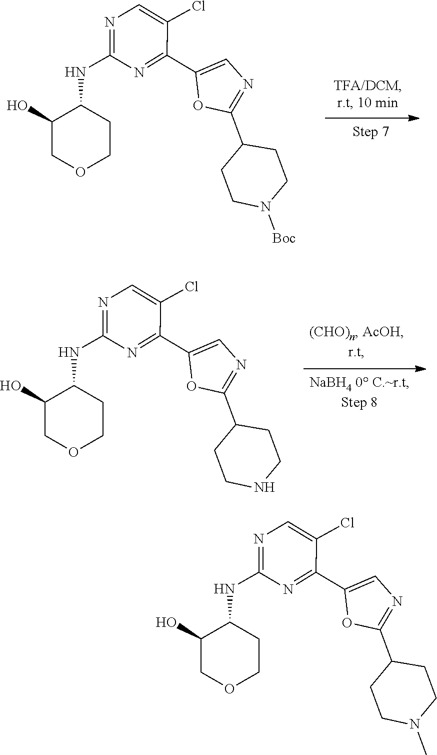

### benzyl 4-((2,2-dimethoxyethyl)carbamoyl)piperidine-1-carboxylate

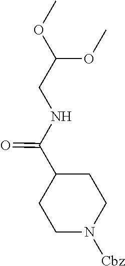 [SMILES: COC(CNC(=O)C1CCN(C(=O)OCc2ccccc2)CC1)OC]

Step 1. To a mixture of 1-((benzyloxy)carbonyl)piperidine-4-carboxylic acid (20 g, 76 mmol) and 2,2-dimethoxyethan-1-amine (8.8 g, 84 mmol) and DIEA (20 g, 0.15 mol) in DMF (200 mL) was added HATU (32 g, 84 mmol) in portions at 25° C. under nitrogen atmosphere. The mixture was stirred for 30 min at 25° C. The solution was purified by reverse flash chromatography with the following conditions: column, C18 silica gel; mobile phase, MeCN in water, 40% to 80% gradient in 20 min; detector, UV 254 nm. Concentration in vacuo resulted in benzyl 4-((2,2-dimethoxyethyl)carbamoyl)piperidine-1-carboxylate (27 g, 91%) as a white solid. m/z (ES⁺) [M+Na]⁺=373.00; HPLC tR=0.642 min.

### 2-(piperidin-4-yl)oxazole

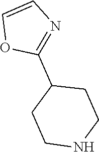 [SMILES: c1coc(C2CCNCC2)n1]

Step 2. A round bottom flask was charged with benzyl 4-((2,2-dimethoxyethyl)carbamoyl)piperidine-1-carboxylate (10 g, 29 mmol), 39394-84-8 (100 mL) and a stirbar before being evacuated and purged with nitrogen three times, and the mixture was stirred for 5 h at 130° C. The residue was quenched with water, then adjusted to pH 10-12 with NaOH at 0° C., and the aqueous phase was extracted with EA (100 mL) three times, and the aqueous phase was extracted with EA/THF(1:4)(100 mL) three times The combined organic layers were washed with brine, dried over sodium sulfate, filtered and concentrated in vacuo. Concentration in vacuo resulted in 2-(piperidin-4-yl)oxazole (1.1 g, 25%) as a yellow oil. m/z (ES⁺) [M+H]⁺=153.30; HPLC tR=0.343 min.

### tert-butyl 4-(oxazol-2-yl)piperidine-1-carboxylate

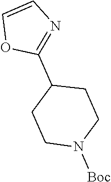 [SMILES: CC(C)(C)OC(=O)N1CCC(c2ncco2)CC1]

Step 3. A resealable reaction vial was charged with 2-(piperidin-4-yl)oxazole (1.1 g, 7.2 mmol), Boc₂O (1.9 g, 8.7 mmol), Na₂CO₃ (0.92 g, 8.7 mmol), THF/H₂O (10 mL) and a stirbar before being evacuated and purged with nitrogen three times, and the mixture was stirred for 30 min at 25° C. The solution was purified by reverse flash chromatography with the following conditions: column, C18 silica gel; mobile phase, MeCN in water, 10% to 50% gradient in 20 min; detector, UV 254 nm. Concentration in vacuo resulted in tert-butyl 4-(oxazol-2-yl)piperidine-1-carboxylate (800 mg, 44%) as a colorless oil. m/z (ES⁺) [M-tBu+H]⁺=197.10; HPLC tR=0.938 min.

### tert-butyl 4-(5-(4,4,5,5-tetramethyl-1,3,2-dioxaborolan-2-yl)oxazol-2-yl)piperidine-1-carboxylate

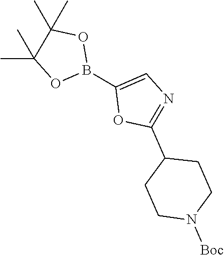 [SMILES: CC(C)(C)OC(=O)N1CCC(c2ncc(B3OC(C)(C)C(C)(C)O3)o2)CC1]

Step 4. To a mixture of tert-butyl 4-(oxazol-2-yl)piperidine-1-carboxylate (1.3 g, 5.2 mmol) in THF (10 mL) was added nBuLi (5.2 mL, 13 mmol) dropwise at −78° C. under nitrogen atmosphere. The mixture was stirred for 20 min at −78° C. prior addition of 2-isopropoxy-4,4,5,5-tetramethyl-1,3,2-dioxaborolane (1.9 g, 10 mmol). The mixture was stirred for 2 h at −40° C. The solution was purified by reverse flash chromatography with the following conditions: column, C18 silica gel; mobile phase, MeCN in water, 10% to 50% gradient in 20 min; detector, UV 254 nm. Concentration in vacuo resulted in tert-butyl 4-(5-(4,4,5,5-tetramethyl-1,3,2-dioxaborolan-2-yl)oxazol-2-yl)piperidine-1-carboxylate (1.3 g, 67%) as a white solid. m/z (ES⁺) [M+H]⁺=379.35; HPLC tR=0.545 min.

### tert-butyl 4-(5-(2,5-dichloropyrimidin-4-yl)oxazol-2-yl)piperidine-1-carboxylate

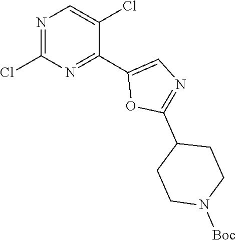 [SMILES: CC(C)(C)OC(=O)N1CCC(c2ncc(-c3nc(Cl)ncc3Cl)o2)CC1]

Step 5. A resealable reaction vial was charged with tert-butyl 4-(5-(4,4,5,5-tetramethyl-1,3,2-dioxaborolan-2-yl)oxazol-2-yl)piperidine-1-carboxylate (2 g, 5 mmol), 2,4,5-trichloropyrimidine (1 g, 8 mmol), Na₂CO₃ (2 g, 0.02 mol), Pd(dppf)Cl₂ (0.4 g, 0.5 mmol), 1,4-Dioxane/H₂O (20 mL) and a stirbar before being evacuated and purged with nitrogen three times, and the mixture was stirred for 1 h at 60° C. The mixture was purified by reverse flash chromatography with the following conditions: column, C18 silica gel; mobile phase, MeCN in water, 10% to 50% gradient in 20 min; detector, UV 254 nm. Concentration in vacuo resulted in tert-butyl 4-(5-(2,5-dichloropyrimidin-4-yl)oxazol-2-yl)piperidine-1-carboxylate (1 g, 50%) as a white solid. m/z (ES⁺) [M+H]⁺=399.15; HPLC tR=0.883 min.

### tert-butyl 4-(5-(5-chloro-2-(((3S,4R)-3-hydroxytetrahydro-2H-pyran-4-yl)amino)pyrimidin-4-yl)oxazol-2-yl)piperidine-1-carboxylate

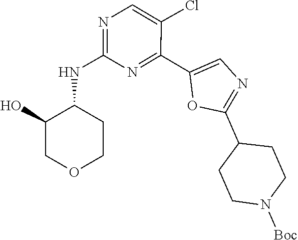 [SMILES: CC(C)(C)OC(=O)N1CCC(c2ncc(-c3nc(N[C@@H]4CCOC[C@H]4O)ncc3Cl)o2)CC1]

Step 6. A resealable reaction vial was charged with tert-butyl 4-(5-(2,5-dichloropyrimidin-4-yl)oxazol-2-yl)piperidine-1-carboxylate (500 mg, 1.25 mmol), (3S,4R)-4-aminotetrahydro-2H-pyran-3-ol (161 mg, 1.38 mmol), DIEA (809 mg, 6.26 mmol), NMP (4 mL) and a stirbar before being evacuated and purged with nitrogen three times, and the mixture was stirred for 1 h at 150° C. The solution was purified by reverse flash chromatography with the following conditions: column, C18 silica gel; mobile phase, MeCN in water, 10% to 50% gradient in 20 min; detector, UV 254 nm. Concentration in vacuo resulted in tert-butyl 4-(5-(5-chloro-2-(((3S,4R)-3-hydroxytetrahydro-2H-pyran-4-yl)amino)pyrimidin-4-yl)oxazol-2-yl)piperidine-1-carboxylate (520 mg, 86.5%) as a white solid. m/z (ES⁺) [M+H]⁺=480.20; HPLC tR=0.820 min.

### (3S,4R)-4-((5-chloro-4-(2-(piperidin-4-yl)oxazol-5-yl)pyrimidin-2-yl)amino)tetrahydro-2H-pyran-3-ol

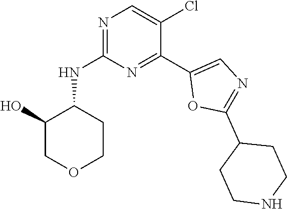 [SMILES: O[C@@H]1COCC[C@H]1Nc1ncc(Cl)c(-c2cnc(C3CCNCC3)o2)n1]

Step 7. A resealable reaction vial was charged with tert-butyl 4-(5-(5-chloro-2-(((3S,4R)-3-hydroxytetrahydro-2H-pyran-4-yl)amino)pyrimidin-4-yl)oxazol-2-yl)piperidine-1-carboxylate (500 mg, 1.04 mmol), DCM/TFA (5 mL) and a stirbar before being evacuated and purged with nitrogen three times, and the mixture was stirred for 10 min at 25° C. The mixture was purified by reverse flash chromatography with the following conditions: column, C18 silica gel; mobile phase, MeCN in water, 10% to 50% gradient in 20 min; detector, UV 254 nm. Concentration in vacuo resulted in (3S,4R)-4-((5-chloro-4-(2-(piperidin-4-yl)oxazol-5-yl)pyrimidin-2-yl)amino)tetrahydro-2H-pyran-3-ol (240 mg, 60.7%) as a white solid. m/z (ES⁺) [M+H-56]⁺=380.15; HPLC tR=0.545 min.

### (3S,4R)-4-((5-chloro-4-(2-(1-methylpiperidin-4-yl)oxazol-5-yl)pyrimidin-2-yl)amino)tetrahydro-2H-pyran-3-ol

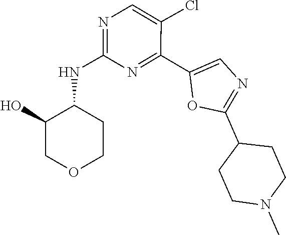 [SMILES: CN1CCC(c2ncc(-c3nc(N[C@@H]4CCOC[C@H]4O)ncc3Cl)o2)CC1]

Step 8 To a mixture of (3S,4R)-4-((5-chloro-4-(2-(piperidin-4-yl)oxazol-5-yl)pyrimidin-2-yl)amino)tetrahydro-2H-pyran-3-ol (200 mg, 0.53 mmol) and AcOH (3.16 mg, 0.05 mmol) in MeOH (3 mL) was added Paraformaldehyde (395 mg, 13.2 mmol) in portions at 25° C. under nitrogen atmosphere. The mixture was stirred for 1 h at 0° C. prior addition of NaBH₄ (299 mg, 7.90 mmol). The mixture was stirred for 30 min at 25° C. The reaction mixture was diluted with H₂O (50 mL), and the aqueous phase was extracted with EA (100 mL) three times. The combined organic layers were washed with brine, dried over sodium sulfate, filtered and concentrated in vacuo. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: MEOH; Flow rate: 60 mL/min mL/min; Gradient: 11% B to 37% B in 7 min; Wavelength: 254 nm/220 nm nm; RT1 (min): 7.38). Lyophilization yielded (3S,4R)-4-((5-chloro-4-(2-(1-methylpiperidin-4-yl)oxazol-5-yl)pyrimidin-2-yl)amino)tetrahydro-2H-pyran-3-ol (14.7 mg, 7.09%) as an amorphous white solid. m/z (ES⁺) [M+H]⁺=394.10; HPLC tR=0.698 min. ¹H NMR (400 MHz, DMSO-d6) δ 8.41 (s, 1H), 8.01 (s, 1H), 7.53 (s, 1H), 4.94 (s, 1H), 3.80 (dt, J=11.1, 5.3 Hz, 3H), 3.49 (s, 1H), 3.03 (t, J=10.4 Hz, 1H), 2.88 (s, 1H), 2.77 (d, J=10.9 Hz, 2H), 2.18 (s, 3H), 2.12-1.95 (m, 5H), 1.89 (s, 1H), 1.77 (qd, J=11.4, 3.6 Hz, 2H), 1.48 (qd, J=12.0, 4.5 Hz, 1H).

### Example 38

### (3S,4R)-4-((2-(1-methylpiperidin-4-yl)-4,6-dihydrothiazolo [5′,4′:5,6]oxepino[4,3-d]pyrimidin-9-yl)amino)tetrahydro-2H-pyran-3-ol (I-1047)

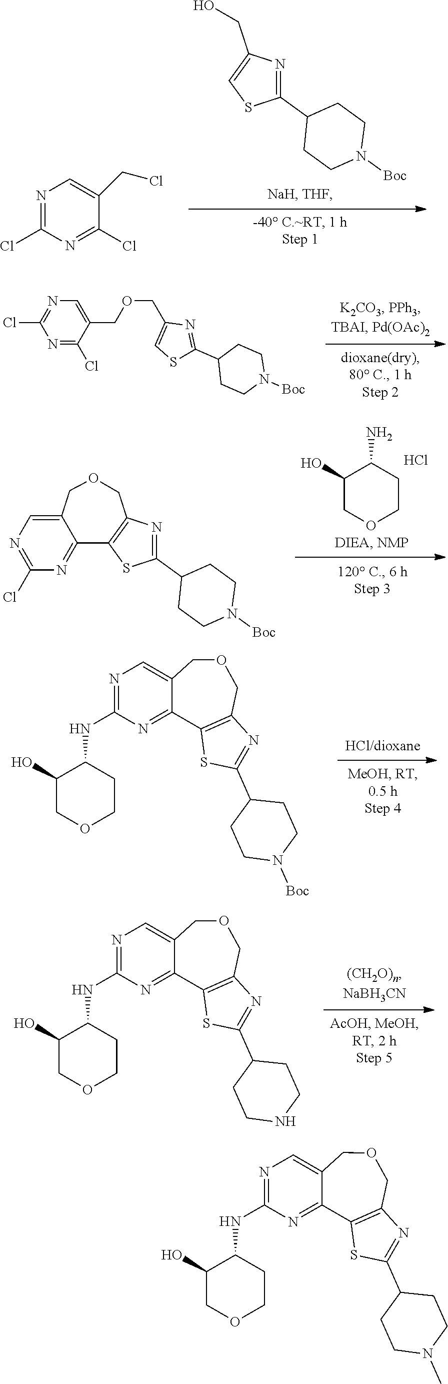

### tert-butyl 4-(4-(((2,4-dichloropyrimidin-5-yl) methoxy) methyl) thiazol-2-yl) piperidine-1-carboxylate

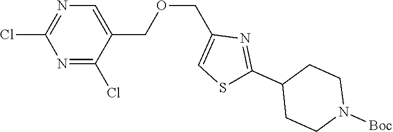 [SMILES: CC(C)(C)OC(=O)N1CCC(c2nc(COCc3cnc(Cl)nc3Cl)cs2)CC1]

Step 1. To a solution of tert-butyl 4-(4-(hydroxymethyl)thiazol-2-yl)piperidine-1-carboxylate (500 mg, 1.68 mmol) in THF (8 mL), NaH (101 mg, 60% Wt, 2.51 mmol) was added at 0° C. under nitrogen atmosphere. Then the mixture was stirred for 30 minutes. The above solution was added dropwise to a solution of 2,4-dichloro-5-(chloromethyl)pyrimidine (397 mg, 2.01 mmol) in THF (2 mL) at −40° C. under nitrogen atmosphere, then the mixture was risen up to RT and stirred for 1 h. The mixture was quenched with saturated NH₄C₁(aq.) and purified by reverse flash chromatography with the following conditions: column, C18 silica gel; mobile phase, MeCN in water, 30% to 90% gradient in 25 min; detector, UV 254 nm. Concentration in vacuo resulted in tert-butyl 4-(4-(((2,4-dichloropyrimidin-5-yl) methoxy) methyl) thiazol-2-yl) piperidine-1-carboxylate (257 mg, 559 mol, 33.4%) as a yellow oil. m/z (ES⁺) [M+H]⁺=459.15; HPLC tR=0.988 min.

### tert-butyl 4-(9-chloro-4,6-dihydrothiazolo [5′,4′:5,6]oxepino[4,3-d]pyrimidin-2-yl) piperidine-1-carboxylate

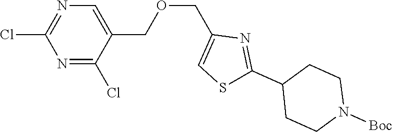 [SMILES: CC(C)(C)OC(=O)N1CCC(c2nc(COCc3cnc(Cl)nc3Cl)cs2)CC1]

Step 2. K₂CO₃ (120 mg, 871 mol), Tetra-n-Butylammoniumlodide (161 mg, 435 mol) and Ph₃P (22.8 mg, 87.1 μmol) were added to a solution of tert-butyl 4-(4-(((2,4-dichloropyrimidin-5-yl) methoxy) methyl) thiazol-2-yl) piperidine-1-carboxylate (200 mg, 435 mol) in 1,4-Dioxane(dry) (4 mL). After bubbling nitrogen through the reaction mixture for 1 minutes, PdOAc₂ (9.77 mg, 43.5 μmol) was added. The reaction mixture was heated at 80° C. for 1 hour with N₂. The reaction was worked up by addition of 8 mL water and extraction with ethyl acetate (5 mL) three times, dried over Na₂SO₄ and evaporated in vacuo. The crude product obtained was purified by Prep-TLC (eluted with a 1/1 petroleum ether/ethyl acetate). After solvent evaporation afforded tert-butyl 4-(9-chloro-4,6-dihydrothiazolo [5′,4′:5,6]oxepino[4,3-d]pyrimidin-2-yl) piperidine-1-carboxylate (80 mg, 0.19 mmol, 43%) as an orange solid. m/z (ES⁺) [M+H]⁺=423.10; HPLC tR=0.863 min.

### tert-butyl 4-(9-(((3S,4R)-3-hydroxytetrahydro-2H-pyran-4-yl) amino)-4,6-dihydrothiazolo [5′,4′: 5,6]oxepino[4,3-d]pyrimidin-2-yl) piperidine-1-carboxylate

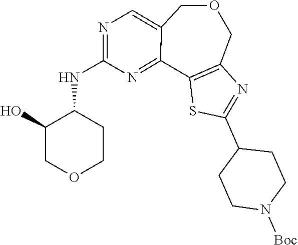 [SMILES: CC(C)(C)OC(=O)N1CCC(c2nc3c(s2)-c2nc(N[C@@H]4CCOC[C@H]4O)ncc2COC3)CC1]

Step 3. To a solution of tert-butyl 4-(9-chloro-4,6-dihydrothiazolo [5′,4′:5,6]oxepino[4,3-d]pyrimidin-2-yl) piperidine-1-carboxylate (65 mg, 0.15 mmol) in NMP (1.4 mL), (3S,4R)-4-aminotetrahydro-2H-pyran-3-ol hydrochloride (47 mg, 0.31 mmol) and DIEA (99 mg, 0.77 mmol) were added. The mixture was heated under 120° C. for 6 h. After cooling, the mixture was purified by flash (Mobile Phase A: Water, Mobile Phase B: ACN; Flow rate: 50 mL/min; Gradient: 40% B to 65% B in 9 min); After solvent evaporation afforded tert-butyl 4-(9-(((3S,4R)-3-hydroxytetrahydro-2H-pyran-4-yl) amino)-4,6-dihydrothiazolo [5′,4′: 5,6]oxepino[4,3-d]pyrimidin-2-yl) piperidine-1-carboxylate (60 mg, 0.12 mmol, 78%) as a yellow solid. m/z (ES⁺) [M+H]⁺=504.15, HPLC tR=0.818 min.

### (3S,4R)-4-((2-(piperidin-4-yl)-4,6-Dihydrothiazolo[5′,4′:5,6]oxepino[4,3-d]pyrimidin-9-yl) amino)tetrahydro-2H-pyran-3-ol

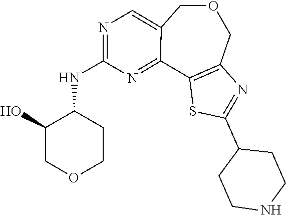 [SMILES: O[C@@H]1COCC[C@H]1Nc1ncc2c(n1)-c1sc(C3CCNCC3)nc1COC2]

Step 4. To a solution of tert-butyl 4-(9-(((3S,4R)-3-hydroxytetrahydro-2H-pyran-4-yl) amino)-4,6-dihydrothia zolo [5′,4′:5,6]oxepino[4,3-d]pyrimidin-2-yl) piperidine-1-carboxylate (55 mg, 0.11 mmol) in MeOH (0.5 mL), HCl/dioxane (0.5 mL, 4N) was added. The mixture was stirred at room temperature for 0.5 hour. The solution was concentrated and adjusted PH=8 by NaHCO₃ aqueous solution. After solvent evaporation afforded (3S,4R)-4-((2-(piperidin-4-yl)-4,6-Dihydrothiazolo[5′,4′:5,6]oxepino[4,3-d]pyrimidin-9-yl) amino)tetrahydro-2H-pyran-3-ol (100 mg) crude as a colorless solid. m/z (ES⁺) [M+H]⁺=404.25; HPLC tR=0.105 min.

### (3S,4R)-4-((2-(1-methylpiperidin-4-yl)-4,6-dihydrothiazolo [5′,4′:5,6]oxepino[4,3-d]pyrimidin-9-yl)amino)tetrahydro-2H-pyran-3-ol

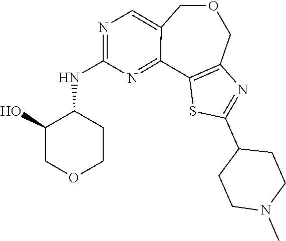 [SMILES: CN1CCC(c2nc3c(s2)-c2nc(N[C@@H]4CCOC[C@H]4O)ncc2COC3)CC1]

Step 5. To a solution of (3S,4R)-4-((2-(piperidin-4-yl)-4,6-dihydrothiazolo[5′,4′:5,6]oxepino[4,3-d]pyrimidin-9-yl)amino)tetrahydro-2H-pyran-3-ol (40 mg, 99 mol, crude) in MeOH (2 mL), Paraformaldehyde (45 mg, 1.5 mmol), Sodium cyanoborohydride (62 mg, 0.99 mmol) and 1 drop of AcOH were added. The mixture was stirred at room temperature for 2 h. The crude residue was purified by Prep-HPLC (Column: YMC-Actus Triart C18, 19×250 mm, 5 μm; Mobile Phase A: Water (10 mmol/L NH₄HCO₃)+0.05% NH₃·H₂O, Mobile Phase B: ACN; Flow rate: 25 mL/min mL/min; Gradient: 33% B to 58% B in 7 min; Wavelength: 254 nm/220 nm nm; RT1 (min): 7.07); Lyophilization yielded (3S,4R)-4-((2-(1-methylpiperidin-4-yl)-4,6-dihydrothiazolo [5′,4′:5,6]oxepino[4,3-d]pyrimidin-9-yl)amino)tetrahydro-2H-pyran-3-ol (14.6 mg, 35.0 μmol, 35%) as a white solid. [M+H]⁺=418.10; HPLC tR=0.567 min. ¹H NMR (400 MHz, DMSO-d) δ 8.14 (s, 1H), 7.13 (d, J=7.8 Hz, 1H), 5.07 (s, 2H), 4.94 (d, J=5.3 Hz, 1H), 4.63 (s, 2H), 3.86-3.73 (m, 3H), 3.49 (s, 1H), 3.34 (s, 1H), 3.04 (t, J=10.4 Hz, 1H), 2.91 (ddt, J=11.7, 7.5, 3.6 Hz, 1H), 2.86-2.79 (m, 2H), 2.18 (s, 3H), 1.99 (td, J=11.3, 9.3, 2.6 Hz, 5H), 1.76-1.63 (m, 2H), 1.53-1.39 (m, 1H).

### Example 39

### (3S,4R)-4-((5-chloro-4-(4-methyl-3-(1-methylpiperidin-4-yl)isothiazol-5-yl)pyrimidin-2-yl)amino)tetrahydro-2H-pyran-3-ol (I-1044)

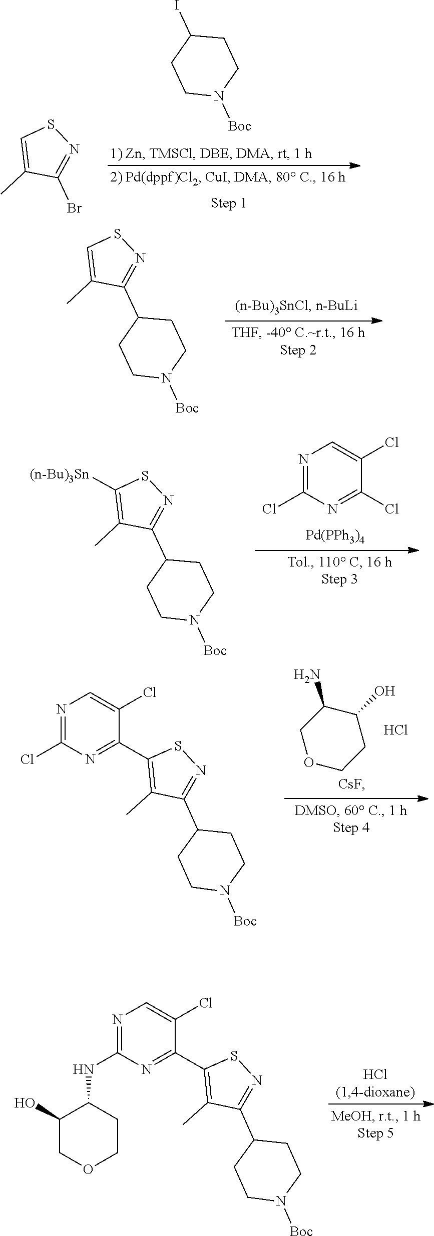

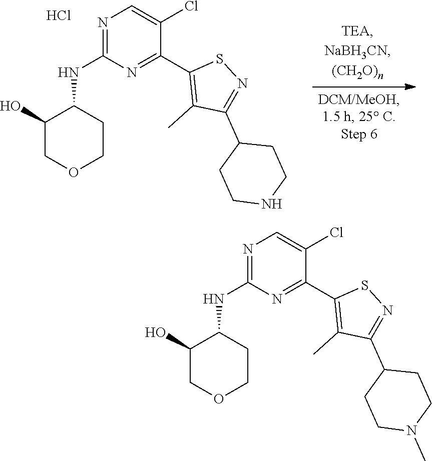

### tert-butyl 4-(4-methylisothiazol-3-yl)piperidine-1-carboxylate

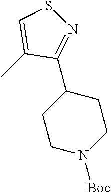 [SMILES: Cc1csnc1C1CCN(C(=O)OC(C)(C)C)CC1]

Step 1. A round bottom flask was charged with zinc (404 mg, 6.18 mmol), DMA (7 mL) and a stirbar before being evacuated and purged with nitrogen three times. 1,2-dibromoethane (52.8 mg, 281 μmol) and TMS-C₁ (30.5 mg, 281 μmol) were added. The mixture was stirred at 20° C. for 30 min. Tert-butyl 4-iodopiperidine-1-carboxylate (1.31 g, 4.21 mmol) in DMA (7 mL) was added dropwise into the mixture. The mixture was stirred at 20° C. for 2 hours to result in Zn-reagent. A resealable reaction vial was charged with 3-bromo-4-methylisothiazole (500 mg, 2.81 mmol), Pd(dppf)Cl₂ (205 mg, 281 mol), CuI (53.5 mg, 281 mol), DMA (7 mL) and a stirbar before being evacuated and purged with nitrogen three times. The Zn-reagent was filtered and the filtrate was added into the above solution. The mixture was stirred at 80° C. for 16 hours. The mixture was diluted with water (100 mL), and the aqueous phase was extracted with EA (30 mL) three times. The combined organic layers were washed with brine, dried over sodium sulfate, filtered and concentrated. The residue was purified by Flash (acetonitrile/water). Concentration in vacuo. The resulting crude material was purified by prep-TLC (PE/EA=4:1). Concentration in vacuo resulted in tert-butyl 4-(4-methylisothiazol-3-yl)piperidine-1-carboxylate (110 mg, 390 mol, 13.9%) as a colorless oil. m/z (ES⁺) [M+H]⁺=283.05; HPLC tR=1.021 min.

### tert-butyl 4-(4-methyl-5-(tributylstannyl)isothiazol-3-yl)piperidine-1-carboxylate

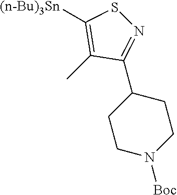

Step 2. A resealable reaction vial was charged with tert-butyl 4-(4-methylisothiazol-3-yl)piperidine-1-carboxylate (110 mg, 390 μmol), THE (2 mL) and a stirbar before being evacuated and purged with nitrogen three times. N-butyllithium (2.5M in hexane, 0.47 mL, 1.17 mmol) was added dropwise at −40° C. The mixture was stirred at −40° C. for 30 min. Tributylchlorostannane (761 mg, 2.34 mmol) was added dropwise at −40° C. The mixture was stirred at 20° C. for 16 hours. The reaction mixture was quenched with sat. NH₄Cl and purified by Flash (acetonitrile/water). Concentration in vacuo yielded tert-butyl 4-(4-methyl-5-(tributylstannyl)isothiazol-3-yl)piperidine-1-carboxylate (65 mg, 0.11 mmol, 29%) as a colorless oil. m/z (ES⁺) [M+H]⁺=573.35; HPLC tR=1.350 min.

### tert-butyl 4-(5-(2,5-dichloropyrimidin-4-yl)-4-methylisothiazol-3-yl)piperidine-1-carboxylate

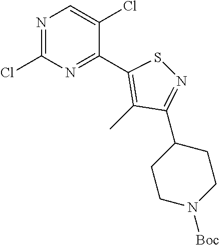 [SMILES: Cc1c(C2CCN(C(=O)OC(C)(C)C)CC2)nsc1-c1nc(Cl)ncc1Cl]

Step 3. A resealable reaction vial was charged with tert-butyl 4-(4-methyl-5-(tributylstannyl)isothiazol-3-yl)piperidine-1-carboxylate (60 mg, 0.10 mmol) in Toluene (1 mL), 2,4,5-trichloropyrimidine (29 mg, 0.16 mmol), and a stirbar before being evacuated and purged with nitrogen three times. Pd(Ph₃P)₄ (24 mg, 21 μmol) was added at 25° C. The resulting mixture was stirred at 110° C. for 16 hours under nitrogen atmosphere. The mixture was cooled to 25° C. The reaction mixture was concentrated in vacuo. The residue was purified by reverse flash chromatography with the following conditions: column, C18 silica gel; mobile phase, ACN in water, 30% to 100% gradient in 20 min; detector, UV 254 nm. Concentrated in vacuo to afford tert-butyl 4-(5-(2,5-dichloropyrimidin-4-yl)-4-methylisothiazol-3-yl)piperidine-1-carboxylate (25 mg, 52 mol, 50%, 90% Purity) as a white solid. m/z (ES⁺) [M+H-tBu]⁺=372.95; HPLC tR=1.218 min.

### tert-butyl 4-(5-(5-chloro-2-(((3S,4R)-3-hydroxytetrahydro-2H-pyran-4-yl)amino)pyrimidin-4-yl)-4-methylisothiazol-3-yl)piperidine-1-carboxylate

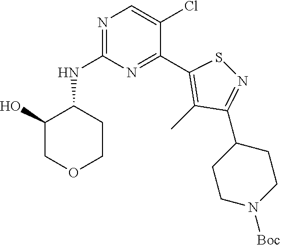 [SMILES: Cc1c(C2CCN(C(=O)OC(C)(C)C)CC2)nsc1-c1nc(N[C@@H]2CCOC[C@H]2O)ncc1Cl]

Step 4. A round bottom flask was charged with tert-butyl 4-(5-(2,5-dichloropyrimidin-4-yl)-4-methylisothiazol-3-yl)piperidine-1-carboxylate (25 mg, 58 mol), (3S,4R)-4-aminotetrahydro-2H-pyran-3-ol hydrochloride (13 mg, 87 μmol), CsF (44 mg, 0.29 mmol) and a stirbar. DMSO (1 mL) was added, and the solution was stirred for 1 hour at 60° C. The mixture was cooled to 25° C. The reaction mixture was diluted with water (2 mL), and the aqueous phase was extracted with EA (6 mL) three times. The combined organic layers were washed with brine, dried over sodium sulfate, filtered, and concentrated in vacuo. The residue was purified by Prep-TLC (ACN/EA; ratio: 1/1) to afford tert-butyl 4-(5-(5-chloro-2-(((3S,4R)-3-hydroxytetrahydro-2H-pyran-4-yl)amino)pyrimidin-4-yl)-4-methylisothiazol-3-yl)piperidine-1-carboxylate (24 mg, 45 μmol, 77%, 95% Purity) as an off-white solid. m/z (ES⁺) [M+H]⁺=510.08; HPLC tR=0.775 min.

### (3S,4R)-4-((5-chloro-4-(4-methyl-3-(piperidin-4-yl)isothiazol-5-yl)pyrimidin-2-yl)amino)tetrahydro-2H-pyran-3-ol hydrochloride

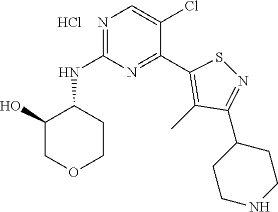 [SMILES: Cc1c(C2CCNCC2)nsc1-c1nc(N[C@@H]2CCOC[C@H]2O)ncc1Cl ; Cl]

Step 5. A round bottomed flask was charged with tert-butyl 4-(5-(5-chloro-2-(((3S,4R)-3-hydroxytetrahydro-2H-pyran-4-yl)amino)pyrimidin-4-yl)-4-methylisothiazol-3-yl)piperidine-1-carboxylate (32 mg, 63 μmol) and a stirbar. MeOH (0.5 mL) and 4M HCl in 1,4-dioxane (0.5 mL) was added, and the solution was stirred for 1 hour at 25° C. The reaction mixture was concentrated in vacuo to afford crude (3S,4R)-4-((5-chloro-4-(4-methyl-3-(piperidin-4-yl)isothiazol-5-yl)pyrimidin-2-yl)amino)tetrahydro-2H-pyran-3-ol hydrochloride (28 mg, 63 μmol, 100%) which was used in the next step directly. m/z (ES⁺) [M+H]⁺=410.20; HPLC tR=0.647 min.

### (3S,4R)-4-((5-chloro-4-(4-methyl-3-(1-methylpiperidin-4-yl)isothiazol-5-yl)pyrimidin-2-yl)amino)tetrahydro-2H-pyran-3-ol

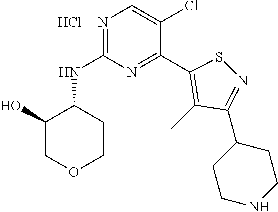 [SMILES: Cc1c(C2CCNCC2)nsc1-c1nc(N[C@@H]2CCOC[C@H]2O)ncc1Cl ; Cl]

Step 6. To a stirred solution of crude (3S,4R)-4-((5-chloro-4-(4-methyl-3-(piperidin-4-yl)isothiazol-5-yl)pyrimidin-2-yl)amino)tetrahydro-2H-pyran-3-ol hydrochloride (28 mg, 63 μmol) in DCM (1 mL) and MeOH (0.2 mL) was added TEA (25 mg, 0.25 mmol) and Paraformaldehyde (5.7 mg, 0.19 mmol). To the reaction was added Sodium cyanoborohydride (12 mg, 0.19 mmol) under 0° C. The reaction was stirred at 25° C. for 1.5 hours. The reaction mixture was concentrated in vacuo. The resulting crude material was purified by Pre-HPLC (Column: Waters Xbridge C18 OBD Column 30×150 mm 5 μm; Mobile Phase A: Water (10 mmol/L NH₄HCO₃+0.5% NH₃H₂O), Mobile Phase B: ACN; Flow rate: 60 mL/min mL/min; Gradient: 17% B to 42% B in 7 min; Wavelength: 254/220 nm; RT1 (min): 6.13). Lyophilization yielded (3S,4R)-4-((5-chloro-4-(4-methyl-3-(1-methylpiperidin-4-yl)isothiazol-5-yl)pyrimidin-2-yl)amino)tetrahydro-2H-pyran-3-ol (10.7 mg, 25.0 μmol, 40% yield, 98.9% purity) as an off-white solid. m/z (ES⁺) [M+H]⁺=424.15; HPLC tR=1.042 min. ¹H NMR (400 MHz, DMSO-d6) 8.48 (s, 1H), 7.60 (d, J=8.2 Hz, 1H), 4.93 (d, J=5.2 Hz, 1H), 3.80 (m, 3H), 3.47 (s, 1H), 3.29 (s, 1H), 3.01 (t, J=10.5 Hz, 1H), 2.86 (d, J=11.4 Hz, 3H), 2.27 (s, 3H), 2.20 (s, 3H), 1.99 (s, 2H), 1.89 (d, J=12.8 Hz, 1H), 1.81 (d, J=8.6 Hz, 4H), 1.47 (m, 1H).

### Example 40

### (3S,4R)-4-((5-chloro-4-(2-((3R,4R)-1,3-dimethylpiperidin-4-yl)thiazol-5-yl)pyrimidin-2-yl)amino)tetrahydro-2H-pyran-3-ol (I-1037)

### (3S,4R)-4-((5-chloro-4-(2-((3S,4S)-1,3-dimethylpiperidin-4-yl)thiazol-5-yl)pyrimidin-2-yl)amino)tetrahydro-2H-pyran-3-ol (I-1038)

### (3S,4R)-4-((5-chloro-4-(2-((3S,4R)-1,3-dimethylpiperidin-4-yl)thiazol-5-yl)pyrimidin-2-yl)amino)tetrahydro-2H-pyran-3-ol (I-1039), &

### (3S,4R)-4-((5-chloro-4-(2-((3R,4S)-1,3-dimethylpiperidin-4-yl)thiazol-5-yl)pyrimidin-2-yl)amino)tetrahydro-2H-pyran-3-ol (I-1040)

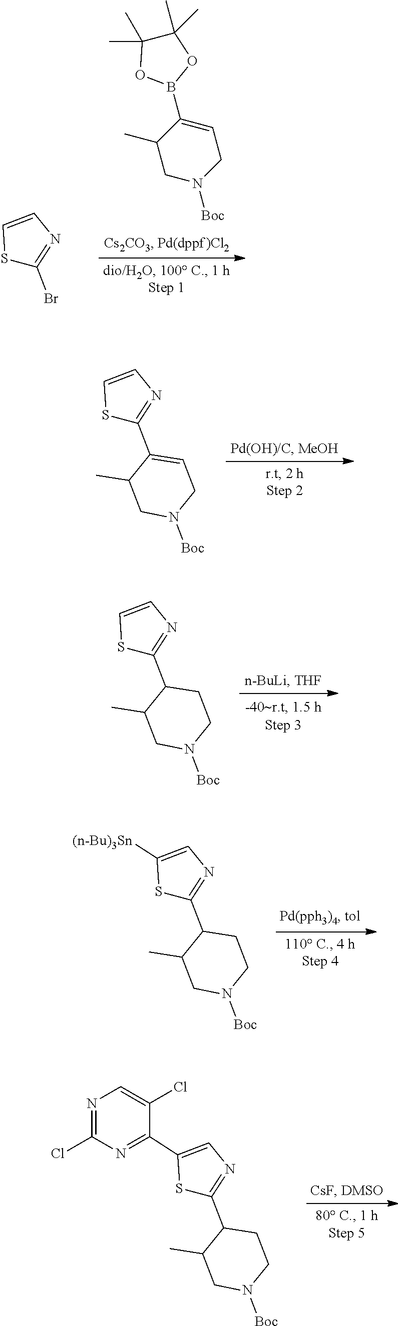

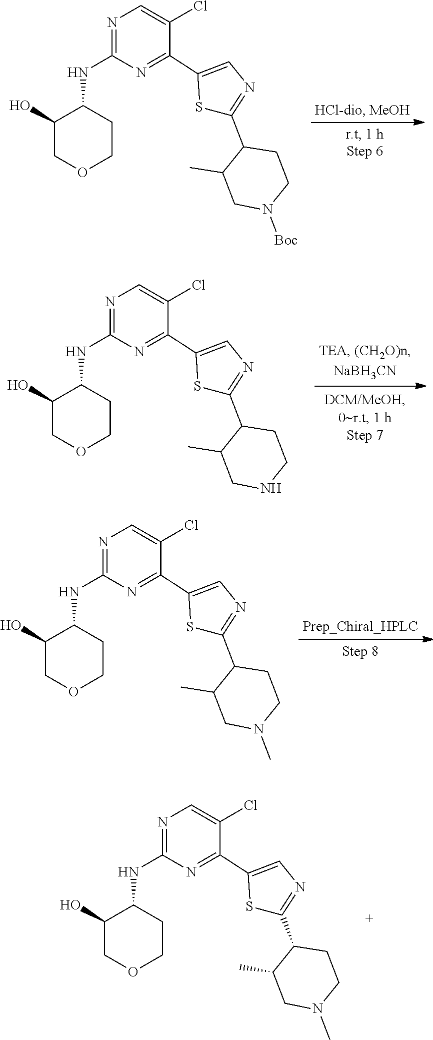 [SMILES: CC1CN(C(=O)OC(C)(C)C)CCC1c1ncc(-c2nc(N[C@@H]3CCOC[C@H]3O)ncc2Cl)s1 ; CC1CNCCC1c1ncc(-c2nc(N[C@@H]3CCOC[C@H]3O)ncc2Cl)s1 ; CC1CN(C)CCC1c1ncc(-c2nc(N[C@@H]3CCOC[C@H]3O)ncc2Cl)s1 ; C[C@H]1CN(C)CC[C@H]1c1ncc(-c2nc(N[C@@H]3CCOC[C@H]3O)ncc2Cl)s1]

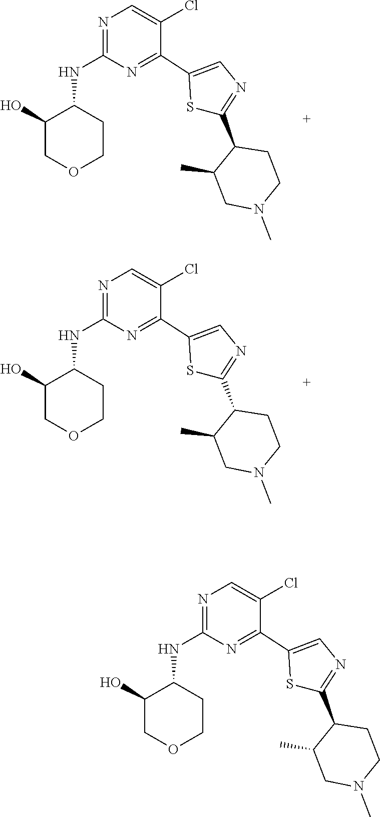 [SMILES: C[C@@H]1CN(C)CC[C@@H]1c1ncc(-c2nc(N[C@@H]3CCOC[C@H]3O)ncc2Cl)s1 ; C[C@@H]1CN(C)CC[C@H]1c1ncc(-c2nc(N[C@@H]3CCOC[C@H]3O)ncc2Cl)s1 ; C[C@H]1CN(C)CC[C@@H]1c1ncc(-c2nc(N[C@@H]3CCOC[C@H]3O)ncc2Cl)s1]

### tert-butyl 3-methyl-4-(thiazol-2-yl)-3,6-dihydropyridine-1(2H)-carboxylate

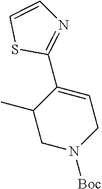 [SMILES: CC1CN(C(=O)OC(C)(C)C)CC=C1c1nccs1]

Step 1. To a solution of tert-butyl 3-methyl-4-(4,4,5,5-tetramethyl-1,3,2-dioxaborolan-2-yl)-3,6-dihydropyridine-1(2H)-carboxylate (2 g, 1 Eq, 6 mmol) in 1,4-Dioxane (1 mL) and H₂O (0.25 mL) were added 2-bromothiazole (1 g, 1.3 Eq, 8 mmol), Cs₂CO₃ (6 g, 3 Eq, 0.02 mol) and Pd(dppf)Cl₂CH₂Cl₂ (0.5 g, 0.1 Eq, 0.6 mmol) under nitrogen atmosphere. The reaction mixture is heated at 100° C. for 1 hour with vigorous stirring. The solution was concentrated under vacuum. The residue was purified by reverse flash chromatography with the following conditions: column, C18 silica gel; mobile phase, MeCN in water, 10% to 70% gradient in 15 min; detector, UV 254 nm to get tert-butyl 3-methyl-4-(thiazol-2-yl)-3,6-dihydropyridine-1(2H)-carboxylate (1.2 g, 3.9 mmol, 60% yield, 90% purity) as a yellow oil. m/z (ES⁺) [M+H]⁺=281.20; HPLC tR=1.023 min.

### tert-butyl 3-methyl-4-(thiazol-2-yl)piperidine-1-carboxylate

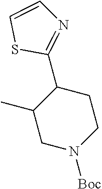 [SMILES: CC1CN(C(=O)OC(C)(C)C)CCC1c1nccs1]

Step 2. A solution of tert-butyl 3-methyl-4-(thiazol-2-yl)-3,6-dihydropyridine-1(2H)-carboxylate (1.2 g, 1 Eq, 4.3 mmol) in MeOH (15 mL) was added Pd/C (0.14 g, 10% Wt, 0.03 Eq, 0.13 mmol) under nitrogen atmosphere before bubbling H₂ through the reaction mixture for 3 times. The mixture was stirred at 25° C. for 2 hours with H₂. The reaction mixture was filtered through a pad of Celite*, the pad was washed with EA, and the filtrate was concentrated in vacuum to get tert-butyl 3-methyl-4-(thiazol-2-yl)piperidine-1-carboxylate (1.1 g, 3.5 mmol, 82% yield, 90% purity) as a yellow oil. m/z (ES⁺) [M+H]⁺=283.15; HPLC tR=0.813 min.

### tert-butyl 3-methyl-4-(5-(tributylstannyl)thiazol-2-yl)piperidine-1-carboxylate

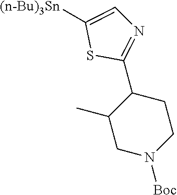

Step 3. To a solution of tert-butyl 3-methyl-4-(thiazol-2-yl)piperidine-1-carboxylate (1.1 g, 1 Eq, 3.9 mmol) in THE (15 mL) was added n-butyllithium (0.75 g, 4.7 mL, 2.5 molar, 3 Eq, 12 mmol) at −40° C. and stirred at −40° C. for 30 min. Then tributylchlorostannane (7.6 g, 6 Eq, 23 mmol) was added, the mixture was stirred at 25° C. for 1 hour. The mixture was quenched with sat. NH₄Cl and concentrated under vacuum. The residue was purified by reverse flash chromatography with the following conditions: column, C18 silica gel; mobile phase, MeCN in water, 50% to 100% gradient in 15 min; detector, UV 254 nm to get tert-butyl 3-methyl-4-(5-(tributylstannyl)thiazol-2-yl)piperidine-1-carboxylate (1.9 g, 3.0 mmol, 77%, 90% Purity) as a yellow oil. m/z (ES⁺) [M+H]⁺=573.10; HPLC tR=1.017 min.

### tert-butyl 4-(5-(2,5-dichloropyrimidin-4-yl)thiazol-2-yl)-3-methylpiperidine-1-carboxylate

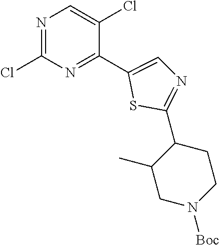 [SMILES: CC1CN(C(=O)OC(C)(C)C)CCC1c1ncc(-c2nc(Cl)ncc2Cl)s1]

Step 4. To a solution of tert-butyl 3-methyl-4-(5-(tributylstannyl)thiazol-2-yl)piperidine-1-carboxylate (1.9 g, 1 Eq, 3.3 mmol) in Toluene (1 mL) were added 2,4,5-trichloropyrimidine (0.67 g, 1.1 Eq, 3.7 mmol) and Pd(Ph₃P)₄ (0.38 g, 0.1 Eq, 0.33 mmol) under nitrogen atmosphere. The reaction mixture is heated at 110° C. for 4 hours with vigorous stirring. The solution was concentrated under vacuum. The residue was purified by reverse flash chromatography with the following conditions: column, C18 silica gel; mobile phase, MeCN in water, 10% to 70% gradient in 15 min; detector, UV 254 nm to get tert-butyl 4-(5-(2,5-dichloropyrimidin-4-yl)thiazol-2-yl)-3-methylpiperidine-1-carboxylate (1.1 g, 2.3 mmol, 69% yield, 90% purity) as a yellow oil. m/z (ES⁺) [M+H-tBu]⁺=373.05; HPLC tR=1.030 min.

### tert-butyl 4-(5-(5-chloro-2-(((3S,4R)-3-hydroxytetrahydro-2H-pyran-4-yl)amino)pyrimidin-4-yl)thiazol-2-yl)-3-methylpiperidine-1-carboxylate

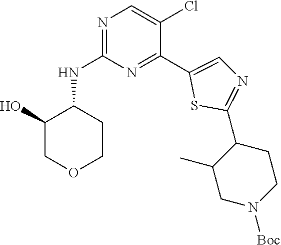 [SMILES: CC1CN(C(=O)OC(C)(C)C)CCC1c1ncc(-c2nc(N[C@@H]3CCOC[C@H]3O)ncc2Cl)s1]

Step 5. To a solution of tert-butyl 4-(5-(2,5-dichloropyrimidin-4-yl)thiazol-2-yl)-3-methylpiperidine-1-carboxylate (600 mg, 1 Eq, 1.40 mmol) in DMSO (1 mL) was added (3S,4R)-4-aminotetrahydro-2H-pyran-3-ol (180 mg, 1.1 Eq, 1.54 mmol), CsF (1.06 g, 5 Eq, 6.99 mmol). The mixture was stirred at 80° C. for 1 hour. The reaction mixture was diluted with water (5 mL), and the aqueous phase was extracted with EA (10 mL) three times. The combined organic layers were washed with brine, dried over sodium sulfate, filtered, and concentrated in vacuum. The residue was purified by reverse flash chromatography with the following conditions: column, C18 silica gel; mobile phase, MeCN in water, 10% to 70% gradient in 15 min; detector, UV 254 nm to get tert-butyl 4-(5-(5-chloro-2-(((3S,4R)-3-hydroxytetrahydro-2H-pyran-4-yl)amino)pyrimidin-4-yl)thiazol-2-yl)-3-methylpiperidine-1-carboxylate (550 mg, 1.0 mmol, 73%, 95% Purity) as a yellow solid. m/z (ES⁺) [M+Na]⁺=532.25; HPLC tR=0.955 min.

### (3S,4R)-4-((5-chloro-4-(2-(3-methylpiperidin-4-yl)thiazol-5-yl)pyrimidin-2-yl)amino)tetrahydro-2H-pyran-3-ol

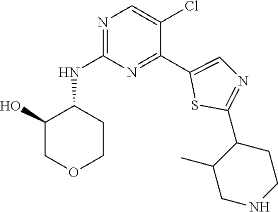 [SMILES: CC1CNCCC1c1ncc(-c2nc(N[C@@H]3CCOC[C@H]3O)ncc2Cl)s1]

Step 6. A solution of tert-butyl 4-(5-(5-chloro-2-(((3S,4R)-3-hydroxytetrahydro-2H-pyran-4-yl)amino)pyrimidin-4-yl)thiazol-2-yl)-3-methylpiperidine-1-carboxylate (530 mg, 1 Eq, 1.04 mmol) in HCl-dioxane (4 mL, 4 molar) and MeOH (2 mL) was stirred at 25° C. for 1 hour. The solution was concentrated under vacuum to get (3S,4R)-4-((5-chloro-4-(2-(3-methylpiperidin-4-yl)thiazol-5-yl)pyrimidin-2-yl)amino)tetrahydro-2H-pyran-3-ol (550 mg, 0.94 mmol, 90%, 70% Purity) as a white solid. m/z (ES⁺) [M+H]⁺=410.15; HPLC tR=0.663 min.

### (3S,4R)-4-((5-chloro-4-(2-(1,3-dimethylpiperidin-4-yl)thiazol-5-yl)pyrimidin-2-yl)amino)tetrahydro-2H-pyran-3-ol

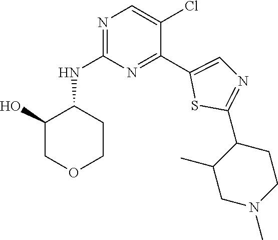 [SMILES: CC1CN(C)CCC1c1ncc(-c2nc(N[C@@H]3CCOC[C@H]3O)ncc2Cl)s1]

Step 7. To a solution of (3S,4R)-4-((5-chloro-4-(2-(3-methylpiperidin-4-yl)thiazol-5-yl)pyrimidin-2-yl)amino)tetrahydro-2H-pyran-3-ol (420 mg, 1 Eq, 1.02 mmol) in DCM (10 mL) and MeOH (2 mL) was added (CH₂O)n (92.3 mg, 3 Eq, 3.07 mmol), sodium cyanoborohydride (322 mg, 298 μL, 5 Eq, 5.12 mmol), TEA (415 mg, 571 μL, 4 Eq, 4.10 mmol) at 0° C. The mixture was stirred at 25° C. for 1 hour. The solution was concentrated under vacuum. The resulting crude material was purified by Pre-HPLC (Column: XBridge Prep OBD C18 Column, 19×250 mm, 5 μm; Mobile Phase A: Water (10 mmol/L NH₄HCO₃+0.05% NH₃H₂O), Mobile Phase B: MEOH; Flow rate: 25 mL/min mL/min; Gradient: 52% B to 68% B in 10 min; Wavelength: 254 nm/220 nm nm; RT1 (min): 9.5; Number Of Runs: 9). Lyophilization yielded (3S,4R)-4-((5-chloro-4-(2-(1,3-dimethylpiperidin-4-yl)thiazol-5-yl)pyrimidin-2-yl)amino)tetrahydro-2H-pyran-3-ol (160 mg, 0.36 mmol, 35%, 95% Purity) as an off-white solid. m/z (ES⁺) [M+H]⁺=424.15; HPLC tR=0.622 min.

### (3S,4R)-4-((5-chloro-4-(2-((3R,4R)-1,3-dimethylpiperidin-4-yl)thiazol-5-yl)pyrimidin-2-yl)amino)tetrahydro-2H-pyran-3-ol

### (3S,4R)-4-((5-chloro-4-(2-((3S,4S)-1,3-dimethylpiperidin-4-yl)thiazol-5-yl)pyrimidin-2-yl)amino)tetrahydro-2H-pyran-3-ol

### (3S,4R)-4-((5-chloro-4-(2-((3S,4R)-1,3-dimethylpiperidin-4-yl)thiazol-5-yl)pyrimidin-2-yl)amino)tetrahydro-2H-pyran-3-ol, &

### (3S,4R)-4-((5-chloro-4-(2-((3R,4S)-1,3-dimethylpiperidin-4-yl)thiazol-5-yl)pyrimidin-2-yl)amino)tetrahydro-2H-pyran-3-ol

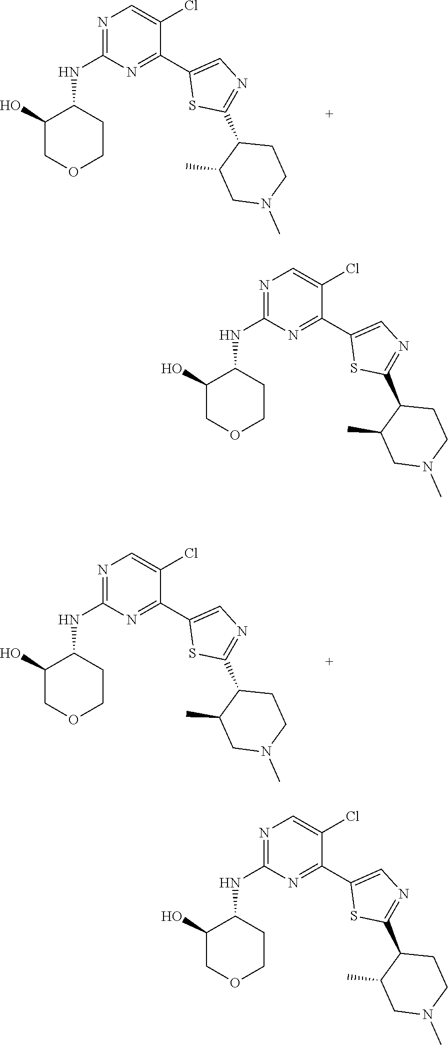

Step 8. A first portion of the resulting material was purified by chiral Pre-HPLC (Column: CHIRALPAK IE, 2×25 cm, 5 μm; Mobile Phase A: Hex(0.5% 2M NH₃-MeOH), Mobile Phase B: EtOH:DCM=1:1-HPLC; Flow rate: 20 mL/min; Gradient: 40% B to 40% B in 18 min; Wavelength: 220/254 nm; Sample Solvent: EtOH:DCM=1:1-HPLC; Injection Volume: 2.0 mL).

Elution at peak 1, and lyophilization yielded (3S,4R)-4-((5-chloro-4-(2-((3R,4R)-1,3-dimethylpiperidin-4-yl)thiazol-5-yl)pyrimidin-2-yl)amino)tetrahydro-2H-pyran-3-ol (I-1037, 5.1 mg, 12 mol, 3.2% yield, 99.9% Purity) as an off-white solid. m/z (ES⁺) [M+H]⁺=424.15; HPLC tR=1.042 min. ¹H NMR (400 MHz, DMSO-d₆) 8.68 (s, 1H), 8.41 (s, 1H), 7.46 (s, 1H), 4.93 (d, J=5.3 Hz, 1H), 3.82 (dd, J=11.4, 5.7 Hz, 3H), 3.50 (s, 1H), 3.34 (s, 1H), 3.24 (s, 1H), 3.05 (d, J=10.6 Hz, 1H), 2.86 (s, 1H), 2.67 (p, J=1.8 Hz, 1H), 2.35-2.11 (m, 5H), 1.96 (d, J=54.0 Hz, 4H), 1.47 (qd, J=11.9, 4.4 Hz, 1H), 0.82 (d, J=6.9 Hz, 3H).

Elution at peak 2, and lyophilization yielded (3S,4R)-4-((5-chloro-4-(2-((3S,4S)-1,3-dimethylpiperidin-4-yl)thiazol-5-yl)pyrimidin-2-yl)amino)tetrahydro-2H-pyran-3-ol (I-1038, 3.7 mg, 8.7 μmol, 2.3% yield, 99.9% Purity) as an off-white solid. m/z (ES⁺) [M+H]⁺=424.15; HPLC tR=1.033 min. ¹H NMR (400 MHz, DMSO-d₆) 8.68 (s, 1H), 8.41 (s, 1H), 7.46 (s, 1H), 4.93 (d, J=5.3 Hz, 1H), 3.82 (dd, J=11.6, 5.8 Hz, 3H), 3.50 (s, 2H), 3.21 (s, 1H), 3.04 (s, 1H), 2.86 (s, 1H), 2.71-2.63 (m, 1H), 2.22 (d, J=30.6 Hz, 5H), 2.11-1.78 (m, 4H), 1.47 (tt, J=12.0, 6.2 Hz, 1H), 0.82 (d, J=6.9 Hz, 3H).

A second portion of the resulting material was purified by chiral Pre-HPLC (Column: CHIRAL ART Cellulose-SC, 2×25 cm, 5 μm; Mobile Phase A: Hex(0.5% 2M NH₃-MeOH)—-HPLC, Mobile Phase B: ETOH:DCM=11-HPLC; Flow rate: 20 mL/min; Gradient: isocratic 30; Wavelength: 220/254 nm; Sample Solvent: EtOH:DCM=1:1-HPLC; Injection Volume: 0.5 mL).

Elution at peak 1, and lyophilization yielded (3S,4R)-4-((5-chloro-4-(2-((3S,4R)-1,3-dimethylpiperidin-4-yl)thiazol-5-yl)pyrimidin-2-yl)amino)tetrahydro-2H-pyran-3-ol (I-1039, 47.6 mg, 110 μmol, 29.2% yield, 98.3% Purity) as an off-white solid. m/z (ES⁺) [M+H]⁺=424.10; HPLC tR=1.242 min. ¹H NMR (400 MHz, DMSO-d₆) 8.65 (s, 1H), 8.41 (s, 1H), 7.45 (s, 1H), 4.93 (d, J=5.4 Hz, 1H), 3.82 (dd, J=10.8, 5.0 Hz, 3H), 3.51 (s, 1H), 3.41 (s, 1H), 3.06 (d, J=10.8 Hz, 1H), 2.85 (dd, J=11.1, 4.6 Hz, 2H), 2.71-2.60 (m, 1H), 2.19 (s, 3H), 2.03-1.86 (m, 4H), 1.84-1.63 (m, 2H), 1.55-1.41 (m, 1H), 0.76 (d, J=6.5 Hz, 3H).

Elution at peak 2, and lyophilization yielded (3S,4R)-4-((5-chloro-4-(2-((3R,4S)-1,3-dimethylpiperidin-4-yl)thiazol-5-yl)pyrimidin-2-yl)amino)tetrahydro-2H-pyran-3-ol (I-1040, 56.4 mg, 0.13 mmol, 35% yield, 99% purity) as an off-white solid. m/z (ES⁺) [M+H]⁺=424.10; HPLC tR=1.258 min. ¹H NMR (400 MHz, DMSO-d₆) 8.66 (s, 1H), 8.41 (s, 1H), 7.45 (s, 1H), 4.92 (d, J=5.3 Hz, 1H), 3.82 (dd, J=11.2, 5.5 Hz, 3H), 3.50 (d, J=11.2 Hz, 1H), 3.41 (s, 1H), 3.09-2.99 (m, 1H), 2.90-2.79 (m, 2H), 2.70-2.59 (m, 1H), 2.19 (s, 3H), 2.01-1.86 (m, 4H), 1.80 (td, J=12.3, 3.6 Hz, 1H), 1.67 (t, J=11.1 Hz, 1H), 1.48 (qd, J=12.0, 4.5 Hz, 1H), 0.76 (d, J=6.5 Hz, 3H).

### Example 41

### (3R,4R)-4-((5-chloro-4-(2-(1-methylpiperidin-4-yl)oxazol-5-yl)pyridin-2-yl)amino)-1-(methylsulfonyl)piperidin-3-ol (I-1030)

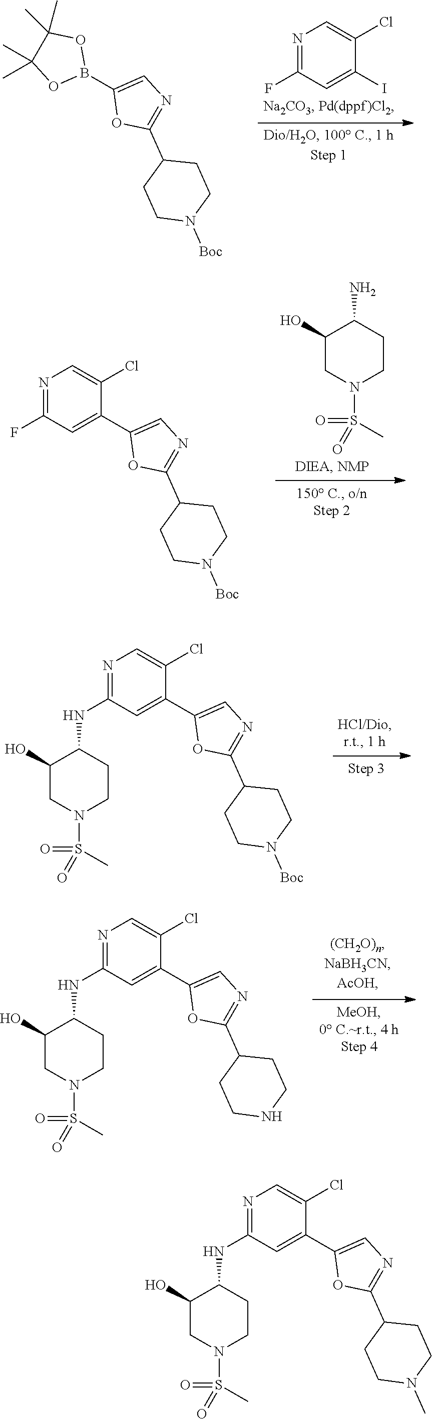

### tert-butyl 4-(5-(5-chloro-2-fluoropyridin-4-yl)oxazol-2-yl)piperidine-1-carboxylate

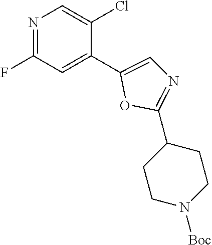 [SMILES: CC(C)(C)OC(=O)N1CCC(c2ncc(-c3cc(F)ncc3Cl)o2)CC1]

Step 1. A resealable reaction vial was charged with tert-butyl 4-(5-(4,4,5,5-tetramethyl-1,3,2-dioxaborolan-2-yl)oxazol-2-yl)piperidine-1-carboxylate (1.00 g, 2.64 mmol), 5-chloro-2-fluoro-4-iodopyridine (817 mg, 3.17 mmol), PdCl₂(dppf)-CH₂Cl₂ (648 mg, 793 mol), Na₂CO₃ (841 mg, 7.93 mmol), 1,4-Dioxane (0.8 mL) and Water (0.2 mL) and a stirbar before being evacuated and purged with nitrogen three times, and the mixture was stirred for 1 hour at 100° C. The solution was purified by reverse flash chromatography with the following conditions: column, C18 silica gel; mobile phase, MeCN in water, 30% to 100% gradient in 20 min; detector, UV 254 nm. Concentration in vacuo resulted in tert-butyl 4-(5-(5-chloro-2-fluoropyridin-4-yl)oxazol-2-yl)piperidine-1-carboxylate (308 mg, 30.5%) as a yellow solid. m/z (ES⁺) [M+H]⁺=382.15; HPLC tR=0.953 min.

### tert-butyl 4-(5-(2-((tert-butoxycarbonyl) ((3R,4R)-3-((tert-butyldimethylsilyl)oxy)-1-(methylsulfonyl)piperidin-4-yl)amino)-5-chloropyridin-4-yl)oxazol-2-yl)piperidine-1-carboxylate

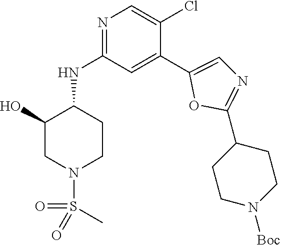 [SMILES: CC(C)(C)OC(=O)N1CCC(c2ncc(-c3cc(N[C@@H]4CCN(S(C)(=O)=O)C[C@H]4O)ncc3Cl)o2)CC1]

Step 2. A round bottomed flask was charged with tert-butyl 4-(5-(5-chloro-2-fluoropyridin-4-yl)oxazol-2-yl)piperidine-1-carboxylate (150 mg, 0.393 mmol), DEA (152 mg, 1.18 mmol), (3R,4R)-4-amino-1-(methylsulfonyl)piperidin-3-ol (153 mg, 0.786 mmol), NMP (0.3 mL) and a stirbar before being evacuated and purged with nitrogen three times, and the solution was stirred for overnight at 150° C. The mixture was purified by reverse flash chromatography with the following conditions: column, C18 silica gel; mobile phase, MeCN in water, 50% to 100% gradient in 20 min; detector, UV 254 nm. Concentration in vacuo resulted in tert-butyl 4-(5-(2-((tert-butoxycarbonyl) ((3R,4R)-3-((tert-butyldimethylsilyl)oxy)-1-(methylsulfonyl)piperidin-4-yl)amino)-5-chloropyridin-4-yl)oxazol-2-yl)piperidine-1-carboxylate (75 mg, 25%) as a yellow solid. m/z (ES⁺) [M+H]⁺=556.35; HPLC tR=0.767 min.

### (3R,4R)-4-((5-chloro-4-(2-(piperidin-4-yl)oxazol-5-yl)pyridin-2-yl)amino)-1-(methylsulfonyl)piperidin-3-ol

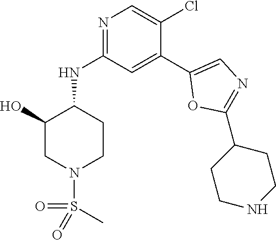 [SMILES: CS(=O)(=O)N1CC[C@@H](Nc2cc(-c3cnc(C4CCNCC4)o3)c(Cl)cn2)[C@H](O)C1]

Step 3. A bottomed round flask was charged with tert-butyl 4-(5-(5-chloro-2-(((3R,4R)-3-hydroxy-1-(methylsulfonyl)piperidin-4-yl)amino)pyridin-4-yl)oxazol-2-yl)piperidine-1-carboxylate (70 mg, 0.13 mmol) in HCl/Dioxane (1 mL) and a stirbar, the solution was stirred for 1 hour at 25° C. The reaction was quenched with NaHCO₃, After filtration, the filtrate was concentrated under reduced pressure. To afford (3R,4R)-4-((5-chloro-4-(2-(piperidin-4-yl)oxazol-5-yl)pyridin-2-yl)amino)-1-(methylsulfonyl)piperidin-3-ol (57 mg, 99%) as a brown solid. m/z (ES⁺) [M+H]⁺=456.25; HPLC tR=0.447 min.

### (3R,4R)-4-((5-chloro-4-(2-(1-methylpiperidin-4-yl)oxazol-5-yl)pyridin-2-yl)amino)-1-(methylsulfonyl)piperidin-3-ol

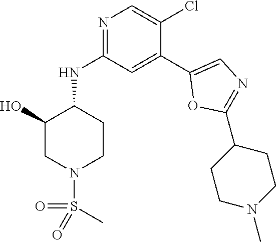 [SMILES: CN1CCC(c2ncc(-c3cc(N[C@@H]4CCN(S(C)(=O)=O)C[C@H]4O)ncc3Cl)o2)CC1]

Step 4. To a mixture of (3R,4R)-4-((5-chloro-4-(2-(piperidin-4-yl)oxazol-5-yl)pyridin-2-yl)amino)-1-(methylsulfonyl)piperidin-3-ol (55 mg, 0.12 mmol) and formaldehyde (36 mg, 1.2 mmol), MeOH (1 mL) and acetic acid (2.2 mg, 2.1 μL, 36 μmol) was added sodium cyanotrihydroborate (76 mg, 1.2 mmol) in portions at 0° C. The mixture was stirred for 4 hours at 25° C. The resulting crude material was purified by Pre-HPLC (Column: XBridge Prep OBD C18 Column, 19×250 mm, 5 μm; Mobile Phase A: Water (10 mmol/LNH₄HCO₃+0.05% NH₃H₂O), Mobile Phase B: MEOH; Flow rate: 25 mL/min mL/min; Gradient: 43% B to 63% B in 10 min; Wavelength: 254 nm/220 nm nm; RT1 (min): 9.5; Number Of Runs: 3). Lyophilization yielded (3R,4R)-4-((5-chloro-4-(2-(1-methylpiperidin-4-yl)oxazol-5-yl)pyridin-2-yl)amino)-1-(methylsulfonyl)piperidin-3-ol (5.6 mg, 9.7%) as a white solid. m/z (ES⁺) [M+H]⁺=470.10; HPLC tR=1.692 min. ¹H NMR (400 MHz, DMSO-d₆) 8.07 (s, 1H), 7.80 (s, 1H), 6.95 (d, J=5.1 Hz, 2H), 5.24 (d, J=4.4 Hz, 1H), 3.75 (s, 1H), 3.59-3.49 (m, 2H), 3.43 (d, J=11.5 Hz, 1H), 2.90 (s, 5H), 2.79-2.66 (m, 3H), 2.18 (s, 3H), 2.12-1.98 (m, 5H), 1.84-1.74 (m, 2H), 1.42 (dd, J=12.0, 8.1 Hz, 1H).

### Example 42

### (3S,4R)-4-((5-chloro-4-(6-(1-methylpiperidin-4-yl)pyrazolo[1,5-b]pyridazin-3-yl)pyrimidin-2-yl)amino)tetrahydro-2H-pyran-3-ol (I-980)

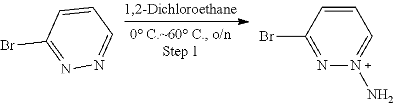 [SMILES: Brc1cccnn1 ; N[n+]1cccc(Br)n1]

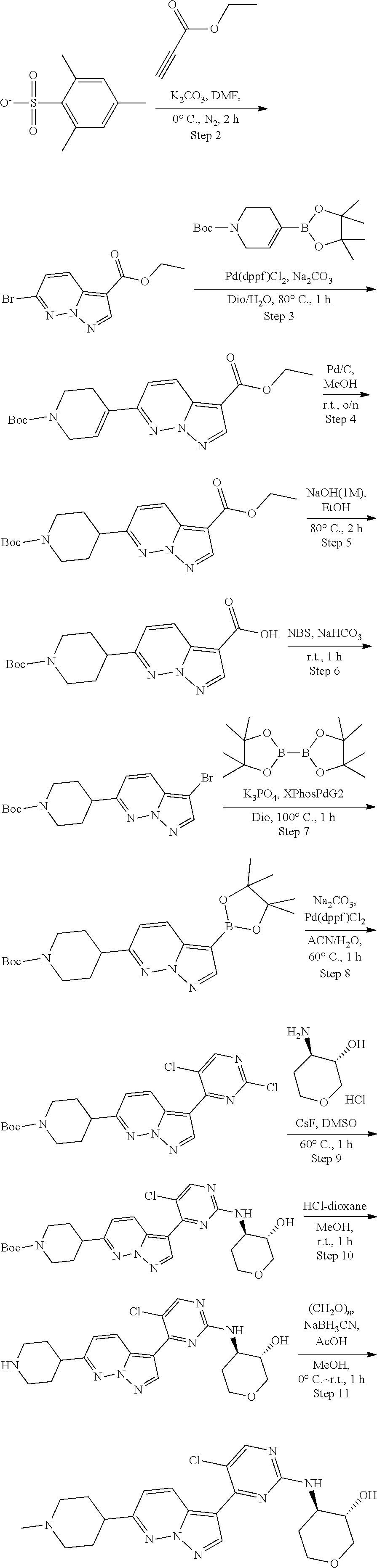

### 1-amino-3-bromopyridazin-1-ium 2,4,6-trimethylbenzenesulfonate

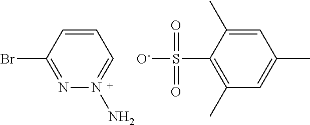

Step 1. To a solution of 3-bromopyridazine (2 g, 0.01 mol) in 1,2-Dichloroethane (50 mL) was added the solution of O-(mesitylsulfonyl)hydroxylamine (5 g, 0.03 mol) in 1,2-Dichloroethane (50 mL) at 25° C. Then the reaction mixture was allowed to stir 16 hours at 20° C. The mixture was concentrated in vacuo to give the crude compound. The crude product was filtered to give the 1-amino-3-bromopyridazin-1-ium 2,4,6-trimethylbenzenesulfonate (15 g, 300%) crude as black solid. m/z (ES⁺) [M+H]⁺=174.0; HPLC tR=0.138 min.

### ethyl 6-bromopyrazolo[1,5-b]pyridazine-3-carboxylate

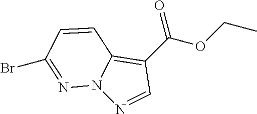

Step 2. A round bottom flask was charged with 1-amino-3-bromopyridazin-1-ium 2,4,6-trimethylbenzenesulfonate (15 g, 40 mmol) and ethyl propiolate (3.50 g, 36 mmol) and K₂CO₃ (17 g, 120 mmol) in DMF (50 mL) and a stirbar, the solution was stirred for 1 hour at 0° C. The reaction was quenched with H₂O and extracted with MTBE (3×100 mL). The combined organic layers were washed with brine (100 mL), dried over anhydrous Na₂SO₄. After filtration, the filtrate was concentrated under reduced pressure. The residue was purified by silica gel column chromatography (eluted with PE/EA; ratio: 15/1) to afford ethyl 6-bromopyrazolo[1,5-b]pyridazine-3-carboxylate (620 mg, 5.7%) as a white solid. m/z (ES⁺) [M+H]⁺=270.00; HPLC tR=0.997 min.

### ethyl 6-(1-(tert-butoxycarbonyl)-1,2,3,6-tetrahydropyridin-4-yl)pyrazolo[1,5-b]pyridazine-3-carboxylate

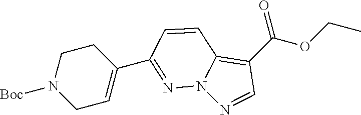 [SMILES: CCOC(=O)c1cnn2nc(C3=CCN(C(=O)OC(C)(C)C)CC3)ccc12]

Step 3. A round bottom flask was charged with ethyl 6-bromopyrazolo[1,5-b]pyridazine-3-carboxylate (520 mg, 1.93 mmol) and tert-butyl 4-(4,4,5,5-tetramethyl-1,3,2-dioxaborolan-2-yl)-3,6-dihydropyridine-1(2H)-carboxylate (893 mg, 2.89 mmol) in 1,4-Dioxane (5 mL) and Water (1 mL) and a stirbar. To the above mixture was added PdCl₂(dppf)-CH₂Cl₂adduct (314 mg, 0.38 mmol) in portions at room temperature under nitrogen atmosphere. The solution was stirred for 1 hour at 80° C. The reaction was diluted with H₂O, extracted with EA (3×10 mL). The combined organic layers were washed with brine (1×10 mL), dried over anhydrous Na₂SO₄. After filtration, the filtrate was concentrated under reduced pressure. The residue was purified by silica gel column chromatography (eluted with PE/EA; ratio: 3/1) to afford ethyl 6-(1-(tert-butoxycarbonyl)-1,2,3,6-tetrahydropyridin-4-yl)pyrazolo[1,5-b]pyridazine-3-carboxylate (700 mg, 97.9%) as a yellow solid. m/z (ES⁺) [M+H]⁺=373.25; HPLC tR=1.138 min.

### ethyl 6-(1-(tert-butoxycarbonyl)piperidin-4-yl)pyrazolo[1,5-b]pyridazine-3-carboxylate

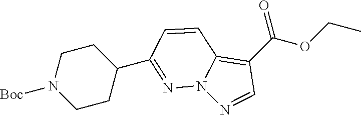 [SMILES: CCOC(=O)c1cnn2nc(C3CCN(C(=O)OC(C)(C)C)CC3)ccc12]

Step 4. To a solution of ethyl 6-(1-(tert-butoxycarbonyl)-1,2,3,6-tetrahydropyridin-4-yl)pyrazolo[1,5-b]pyridazine-3-carboxylate (700 mg, 1.88 mmol) in MeOH (10 mL), 10% Pd/C (70 mg) was added under nitrogen atmosphere. The mixture was hydrogenated at room temperature for 20 hour under hydrogen atmosphere. After filtration, the filtrate was concentrated under reduced pressure to afford ethyl 6-(1-(tert-butoxycarbonyl)piperidin-4-yl)pyrazolo[1,5-b]pyridazine-3-carboxylate (550 mg, 78.1%) as a yellow solid. m/z (ES⁺) [M+H]⁺=375.20; HPLC tR=1.113 min.

### 6-(1-(tert-butoxycarbonyl)piperidin-4-yl)pyrazolo[1,5-b]pyridazine-3-carboxylic acid

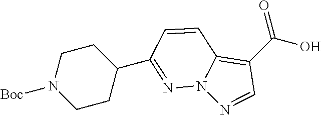 [SMILES: CC(C)(C)OC(=O)N1CCC(c2ccc3c(C(=O)O)cnn3n2)CC1]

Step 5. Around bottom flask was charged with ethyl 6-(1-(tert-butoxycarbonyl)piperidin-4-yl)pyrazolo[1,5-b]pyridazine-3-carboxylate (500 mg, 1.34 mmol) and NaOH (107 mg, 2.67 mL, 1 molar in H₂O, 2.67 mmol) in EtOH (5 mL) and a stirbar, the solution was stirred for 2 hour at 80° C. The mixture was acidified to pH 5 with HCl (1M). The resulting mixture was extracted with DCM (3×3 mL). The combined organic layers were washed with brine (1×3 mL), dried over anhydrous Na₂SO₄, After filtration, the filtrate was concentrated under reduced pressure to afford 6-(1-(tert-butoxycarbonyl)piperidin-4-yl)pyrazolo[1,5-b]pyridazine-3-carboxylic acid (550 mg, 119%) crude as a yellow solid. m/z (ES⁺) [M+Na]⁺=369.05; HPLC tR=0.518 min.

### tert-butyl 4-(3-bromopyrazolo[1,5-b]pyridazin-6-yl)piperidine-1-carboxylate

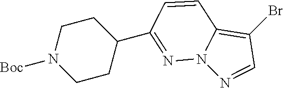 [SMILES: CC(C)(C)OC(=O)N1CCC(c2ccc3c(Br)cnn3n2)CC1]

Step 6. A round bottom flask was charged with 6-(1-(tert-butoxycarbonyl)piperidin-4-yl)pyrazolo[1,5-b]pyridazine-3-carboxylic acid (554 mg, 1.60 mmol) and sodium bicarbonate (403 mg, 4.80 mmol) and NBS (313 mg, 1.76 mmol) in DMF (2 mL) and a stirbar, the solution was stirred for 1 hour at 25° C. The reaction was quenched with H₂O and extracted with EA (3×3 mL). The combined organic layers were washed with brine (1×5 mL), dried over anhydrous Na₂SO₄. After filtration, the filtrate was concentrated under reduced pressure. The residue was purified by silica gel column chromatography (eluted with PE/EA; ratio: 3/1) to afford tert-butyl 4-(3-bromopyrazolo[1,5-b]pyridazin-6-yl)piperidine-1-carboxylate (300 mg, 49.2%) as a yellow solid. m/z (ES⁺) [M+Na]=403.10; HPLC tR=1.130 min.

### tert-butyl 4-(3-(4,4,5,5-tetramethyl-1,3,2-dioxaborolan-2-yl)pyrazolo[1,5-b]pyridazin-6-yl)piperidine-1-carboxylate

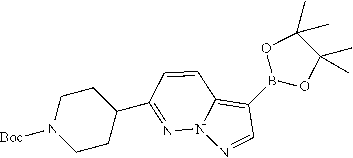 [SMILES: CC(C)(C)OC(=O)N1CCC(c2ccc3c(B4OC(C)(C)C(C)(C)O4)cnn3n2)CC1]

Step 7. A resealable reaction vial was charged with tert-butyl 4-(3-bromopyrazolo[1,5-b]pyridazin-6-yl)piperidine-1-carboxylate (130 mg, 341 mol), Potassium phosphate, tribasic (217 mg, 1.02 mmol), 4,4,4′,4′,5,5,5′,5′-octamethyl-2,2′-bi(1,3,2-dioxaborolane) (520 mg, 2.05 mmol), XPhos Palladacycle (80 mg, 0.102 mmol), 1,4-Dioxane (13 mL) and a stirbar before being evacuated and purged with nitrogen three times, and the mixture was stirred for 1 h at 100° C. After cooling, it was filtered to remove the filter cake and the filtrate was evaporated in vacuo. The residue was purified by reverse flash chromatography with the following conditions: column, C18 silica gel; mobile phase, MeCN in water, 50% to 100% gradient in 20 min; detector, UV 220 nm. Concentration in vacuo resulted in tert-butyl 4-(3-(4,4,5,5-tetramethyl-1,3,2-dioxaborolan-2-yl)pyrazolo[1,5-b]pyridazin-6-yl)piperidine-1-carboxylate (70 mg, 48%) as a white solid. m/z (ES⁺) [M+H]⁺=429.10; HPLC tR=0.833 min.

### tert-butyl 4-(3-(2,5-dichloropyrimidin-4-yl)pyrazolo[1,5-b]pyridazin-6-yl)piperidine-1-carboxylate

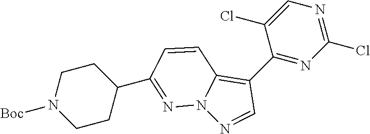 [SMILES: CC(C)(C)OC(=O)N1CCC(c2ccc3c(-c4nc(Cl)ncc4Cl)cnn3n2)CC1]

Step 8. A resealable reaction vial was charged with tert-butyl 4-(3-(4,4,5,5-tetramethyl-1,3,2-dioxaborolan-2-yl)pyrazolo[1,5-b]pyridazin-6-yl)piperidine-1-carboxylate (77 mg, 0.18 mmol), 2,4,5-trichloropyrimidine (33 mg, 0.18 mmol), sodium bicarbonate (45 mg, 0.54 mmol), PdCl₂(dppf)-CH₂Cl₂adduct (15 mg, 0.018 mmol), MeCN (10 mL) and Water (2.0 mL) and a stirbar before being evacuated and purged with nitrogen three times, and the mixture was stirred for 1 hour at 60° C. The residue was purified by reverse flash chromatography with the following conditions: column, C18 silica gel; mobile phase, MeCN in water, 50% to 100% gradient in 20 min; detector, UV 254 nm. Concentration in vacuo resulted in tert-butyl 4-(3-(2,5-dichloropyrimidin-4-yl)pyrazolo[1,5-b]pyridazin-6-yl)piperidine-1-carboxylate (40 mg, 50%) as a yellow solid. m/z (ES⁺) [M+H]⁺=449.00; HPLC tR=0.798 min.

### tert-butyl 4-(3-(5-chloro-2-(((3S,4R)-3-hydroxytetrahydro-2H-pyran-4-yl)amino)pyrimidin-4-yl)pyrazolo[1,5-b]pyridazin-6-yl)piperidine-1-carboxylate

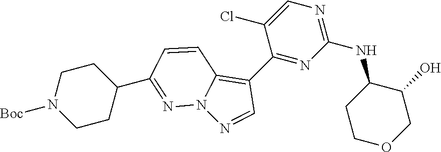 [SMILES: CC(C)(C)OC(=O)N1CCC(c2ccc3c(-c4nc(N[C@@H]5CCOC[C@H]5O)ncc4Cl)cnn3n2)CC1]

Step 9. A resealable reaction vial was charged with tert-butyl 4-(3-(2,5-dichloropyrimidin-4-yl)pyrazolo[1,5-b]pyridazin-6-yl)piperidine-1-carboxylate (67 mg, 0.15 mmol), (3S,4R)-4-aminotetrahydro-2H-pyran-3-ol (21 mg, 0.18 mmol), CsF (0.11 g, 0.75 mmol), DMSO (0.7 mL) and a stirbar before being evacuated and purged with nitrogen three times, and the mixture was stirred for 1 hour at 60° C. The solution was purified by reverse flash chromatography with the following conditions: column, C18 silica gel; mobile phase, MeCN in water, 30% to 100% gradient in 20 min; detector, UV 254 nm, Concentration in vacuo resulted in tert-butyl 4-(3-(5-chloro-2-(((3S,4R)-3-hydroxytetrahydro-2H-pyran-4-yl)amino)pyrimidin-4-yl)pyrazolo[1,5-b]pyridazin-6-yl)piperidine-1-carboxylate (40 mg, 51%) as a white solid. m/z (ES⁺) [M+H]⁺=530.30; HPLC tR=0.913 min.

### (3S,4R)-4-((5-chloro-4-(6-(piperidin-4-yl)pyrazolo[1,5-b]pyridazin-3-yl) pyrimidin-2-yl)amino)tetrahydro-2H-pyran-3-ol

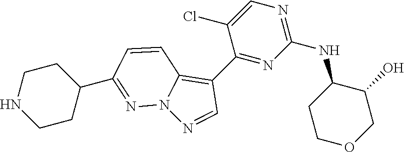 [SMILES: O[C@@H]1COCC[C@H]1Nc1ncc(Cl)c(-c2cnn3nc(C4CCNCC4)ccc23)n1]

Step 10. A round bottom flask was charged with tert-butyl 4-(3-(5-chloro-2-(((3S,4R)-3-hydroxytetrahydro-2H-pyran-4-yl)amino)pyrimidin-4-yl)pyrazolo[1,5-b]pyridazin-6-yl)piperidine-1-carboxylate (40 mg, 75 μmol) and HCl/dioxane (0.4 mL, 4 molar) in MeOH (0.5 mL) and a stirbar, the solution was stirred for 1 hour at 25° C. The solution was concentrated. The mixture was adjusted to pH 8 with aq. NaHCO₃ and was concentrated under reduced pressure to afford (3S,4R)-4-((5-chloro-4-(6-(piperidin-4-yl)pyrazolo[1,5-b]pyridazin-3-yl) pyrimidin-2-yl)amino)tetrahydro-2H-pyran-3-ol (50 mg, crude) as a white solid. m/z (ES⁺) [M+H]⁺=430.45; HPLC tR=0.492 min.

### (3S,4R)-4-((5-chloro-4-(6-(1-methylpiperidin-4-yl)pyrazolo[1,5-b]pyridazin-3-yl)pyrimidin-2-yl)amino)tetrahydro-2H-pyran-3-ol

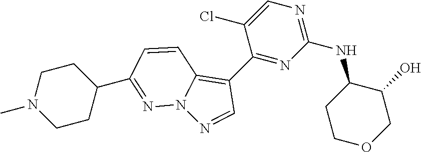 [SMILES: CN1CCC(c2ccc3c(-c4nc(N[C@@H]5CCOC[C@H]5O)ncc4Cl)cnn3n2)CC1]

Step 11. To a mixture of (3S,4R)-4-((5-chloro-4-(6-(piperidin-4-yl)pyrazolo[1,5-b]pyridazin-3-yl)pyrimidin-2-yl)amino)tetrahydro-2H-pyran-3-ol (32 mg, 74 mol) and Paraformaldehyde (22 mg, 0.74 mmol) and sodium cyanotrihydroborate (47 mg, 0.74 mmol) in MeOH (2 mL) was added acetic acid (1.3 mg, 22 mol) in portions at 0° C. The mixture was stirred for 1 h at 25° C. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 15% B to 42% B in 7 min; Wavelength: 254 nm/220 nm nm; RT1 (min): 7.57). Lyophilization yielded (3S,4R)-4-((5-chloro-4-(6-(1-methylpiperidin-4-yl)pyrazolo[1,5-b]pyridazin-3-yl)pyrimidin-2-yl)amino)tetrahydro-2H-pyran-3-ol (9.9 mg, 30%, 99.639% Purity) as a white solid. m/z (ES⁺) [M+H]⁺=444.10; HPLC tR=1.292 min. ¹H NMR (400 MHz, DMSO-d₆) δ 9.19 (s, 1H), 8.89 (s, 1H), 8.35 (s, 1H), 7.53 (s, 2H), 5.04 (s, 1H), 3.89-3.78 (m, 3H), 3.52-3.45 (m, 1H), 3.36-3.35 (s, 1H), 3.09 (s, 1H), 2.88 (dd, J=26.8, 11.4 Hz, 3H), 2.22 (s, 3H), 2.05-1.92 (m, 5H), 1.87-1.77 (m, 2H), 1.52 (s, 1H).

### Example 43

### (3S,4R)-4-((5-chloro-4-(3-(4-methylpiperazin-1-yl)-1,2,4-thiadiazol-5-yl)pyridin-2-yl)amino)tetrahydro-2H-pyran-3-ol (I-1026)

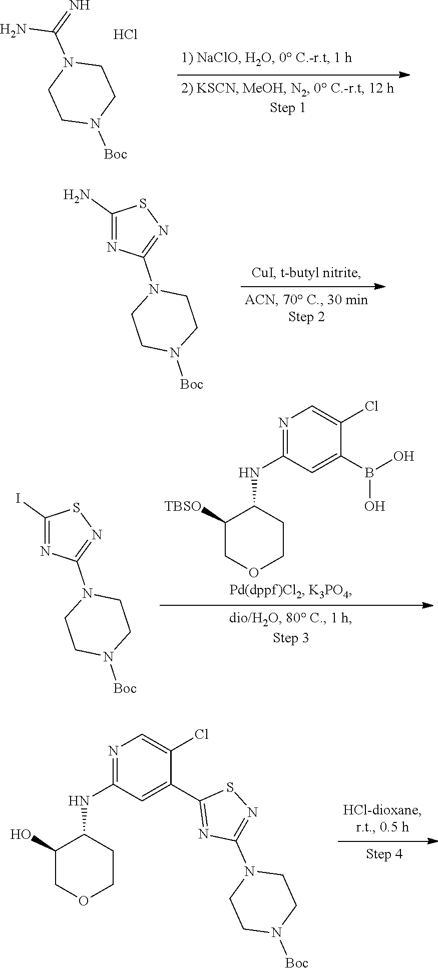 [SMILES: CC(C)(C)OC(=O)N1CCN(C(=N)N)CC1 ; Cl ; CC(C)(C)OC(=O)N1CCN(c2nsc(N)n2)CC1 ; CC(C)(C)OC(=O)N1CCN(c2nsc(I)n2)CC1 ; CC(C)(C)[Si](C)(C)O[C@@H]1COCC[C@H]1Nc1cc(B(O)O)c(Cl)cn1 ; CC(C)(C)OC(=O)N1CCN(c2nsc(-c3cc(N[C@@H]4CCOC[C@H]4O)ncc3Cl)n2)CC1]

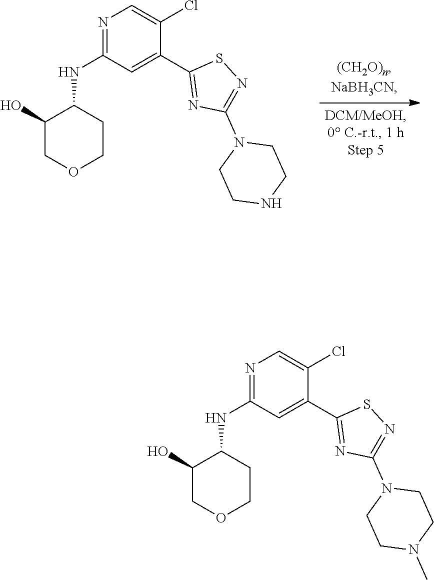 [SMILES: O[C@@H]1COCC[C@H]1Nc1cc(-c2nc(N3CCNCC3)ns2)c(Cl)cn1 ; CN1CCN(c2nsc(-c3cc(N[C@@H]4CCOC[C@H]4O)ncc3Cl)n2)CC1]

### tert-butyl 4-(5-amino-1,2,4-thiadiazol-3-yl)piperazine-1-carboxylate

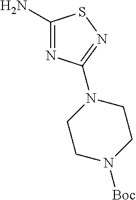 [SMILES: CC(C)(C)OC(=O)N1CCN(c2nsc(N)n2)CC1]

Step 1. To a solution of tert-butyl 4-carbamimidoylpiperazine-1-carboxylate hydrochloride (1 g, 1 Eq, 4 mmol) in water (10 mL) was added sodium hypochlorite (6.1 mL, 0.7M in water) at 0° C. The mixture was stirred at room temperature for 1 h, diluted with water and extracted three times with EtOAc. The combined organic layers were dried over Na₂SO₄, concentrated, filtered and evaporated. The residue was dissolved in MeOH (10 mL) and Potassium thiocyanate (0.4 g, 0.2 mL, 1.1 Eq, 4 mmol) was added at 0° C. with N₂. Then the solution was stirred at room temperature for 12 h and the mixture was concentrated. The residue was dissolved in EtOAc, filtered and the filtrate was concentrated. The residue was purified by silica gel column (PE:EtOAc=2:1-1:3). After solvent evaporation afforded product tert-butyl 4-(5-amino-1,2,4-thiadiazol-3-yl)piperazine-1-carboxylate (120 mg, 421 mol, 10%) as a yellow solid. m/z (ES⁺) [M+H]⁺=286.15; HPLC tR=0.728 min.

### tert-butyl 4-(5-iodo-1,2,4-thiadiazol-3-yl)piperazine-1-carboxylate

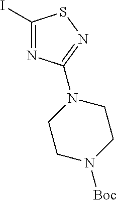 [SMILES: CC(C)(C)OC(=O)N1CCN(c2nsc(I)n2)CC1]

Step 2. To a mixture of tert-butyl 4-(5-amino-1,2,4-thiadiazol-3-yl)piperazine-1-carboxylate (120 mg, 1 Eq, 421 μmol) and copper(I) iodide (320 mg, 4 Eq, 1.68 mmol) in MeCN (2 mL). Tert-butyl nitrite (86.7 mg, 2 Eq, 841 mol) was added dropwise at rt under nitrogen atmosphere. The mixture was stirred for 30 min at 70° C. The resulting mixture was diluted with water and extracted with EtOAc (2×20 ml). The combined organic layers were washed with brine (1×10 mL), dried over anhydrous Na₂SO₄. After filtration, the filtrate was concentrated under reduced pressure. The residue was purified by C18 column chromatography (80 g, eluted with MeCN in water 0-100% in 30 min) to afford tert-butyl 4-(5-iodo-1,2,4-thiadiazol-3-yl)piperazine-1-carboxylate (95 mg, 0.24 mmol, 57%) as a yellow solid. m/z (ES⁺) [M+H-t-Bu]⁺=340.95; HPLC tR=1.005 min.

### tert-butyl 4-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperazine-1-carboxylate

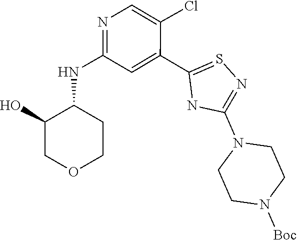

Step 3. To a solution of tert-butyl 4-(5-iodo-1,2,4-thiadiazol-3-yl)piperazine-1-carboxylate (80 mg, 1 Eq, 0.20 mmol) and (2-(((3S,4R)-3-((tert-butyldimethylsilyl)oxy)tetrahydro-2H-pyran-4-yl)amino)-5-chloropyridin-4-yl)boronic acid (0.14 g, 1.8 Eq, 0.36 mmol) in 1,4-Dioxane (1.6 mL) and Water (0.4 mL), Pd(dppf)Cl₂ CH₂Cl₂ (16 mg, 0.1 Eq, 20 mol) and potassium phosphate (0.13 g, 3 Eq, 0.61 mmol) were added. After stirring for 1 hour at 80° C. under a nitrogen atmosphere, the reaction was diluted with water at rt. The resulting mixture was extracted with EtOAc (2×20 ml). The combined organic layers were washed with brine (1×10 mL), dried over anhydrous Na₂SO₄. After filtration, the filtrate was concentrated under reduced pressure. The residue was purified by C 18 chromatography (80 g, eluted with MeCN in water 0-100%) to afford tert-butyl 4-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperazine-1-carboxylate (95 mg, 0.16 mmol, 77%) as a yellow oil. m/z (ES⁺) [M+H]⁺=611.10; HPLC tR=0.950 min.

### (3S,4R)-4-((5-chloro-4-(3-(piperazin-1-yl)-1,2,4-thiadiazol-5-yl)pyridin-2-yl)amino)tetrahydro-2H-pyran-3-ol

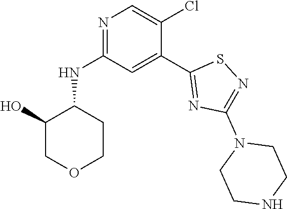 [SMILES: O[C@@H]1COCC[C@H]1Nc1cc(-c2nc(N3CCNCC3)ns2)c(Cl)cn1]

Step 4. A round bottom flask was charged with tert-butyl 4-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperazine-1-carboxylate (90 mg, 1 eq, 0.15 mmol) and HCl-dioxane (10 mL, 4 molar), the solution was stirred for 0.5 h at 28° C. and concentrated under reduced pressure to afford crude (3S,4R)-4-((5-chloro-4-(3-(piperazin-1-yl)-1,2,4-thiadiazol-5-yl)pyridin-2-yl)amino)tetrahydro-2H-pyran-3-ol (55 mg, 0.14 mmol, 94%) as a yellow solid. m/z (ES⁺) [M+H]⁺=397.00; HPLC tR=0.533 min.

### (3S,4R)-4-((5-chloro-4-(3-(4-methylpiperazin-1-yl)-1,2,4-thiadiazol-5-yl)pyridin-2-yl)amino)tetrahydro-2H-pyran-3-ol

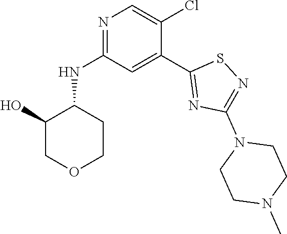 [SMILES: CN1CCN(c2nsc(-c3cc(N[C@@H]4CCOC[C@H]4O)ncc3Cl)n2)CC1]

Step 5. A bottomed round flask was charged with (3S,4R)-4-((5-chloro-4-(3-(piperazin-1-yl)-1,2,4-thiadiazol-5-yl)pyrimidin-2-yl)amino)tetrahydro-2H-pyran-3-ol (50 mg, 1 Eq, 0.13 mmol), paraformaldehyde (19 mg, 19 μL, 5 Eq, 0.63 mmol) and TEA (38 mg, 53 μL, 3 Eq, 0.38 mmol) in DCM (4 mL) and MeOH (0.8 mL), the solution was stirred for 5 min at 0° C. Then the sodium cyanoborohydride (39 mg, 37 μL, 5 Eq, 0.63 mmol) was added in portion. The mixture was warmed up to r.t and stirred for 1 h at 25° C. The resulting mixture was diluted with DCM (2×20 ml) and washed with brine (1×10 mL), dried over anhydrous Na₂SO₄. After filtration, the filtrate was concentrated under reduced pressure. The residue was purified by Prep-TLC (eluted with DCM:MeOH=10:1) to afford crude product 26 mg as a yellow solid. The crude product (26 mg) was purified by Prep-HPLC with the following conditions: (Column: XBridge Shield RP18 OBD Column, 30×150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 25% B to 48% B in 7 min; Wavelength: 254 nm/220 nm nm; RT1 (min): 6.33; Number Of Runs: 1) to afford (3S,4R)-4-((5-chloro-4-(3-(4-methylpiperazin-1-yl)-1,2,4-thiadiazol-5-yl)pyridin-2-yl)amino)tetrahydro-2H-pyran-3-ol (7.5 mg, 18 mol, 15%, 99.9% purity) as a light yellow solid. m/z (ES⁺) [M+H]⁺=411.05; HPLC tR=1.665 min. ¹H NMR (400 MHz, DMSO-d6) 8.20 (s, 1H), 7.48 (s, 1H), 7.15 (d, J=7.5 Hz, 1H), 4.95 (d, J=5.2 Hz, 1H), 3.80 (td, J=11.1, 5.5 Hz, 3H), 3.68 (t, J=5.1 Hz, 4H), 3.45-3.33 (m, 2H), 3.08 (dd, J=11.1, 9.2 Hz, 1H), 2.43 (t, J=5.1 Hz, 4H), 2.24 (s, 3H), 2.05-1.97 (m, 1H), 1.45-1.32 (m, 1H).

### Example 44

### (3S,4R)-4-((5-chloro-4-(6-(1-methylpiperidin-4-yl)pyrazolo[1,5-a]pyridin-3-yl)pyrimidin-2-yl)amino)tetrahydro-2H-pyran-3-ol (I-532)

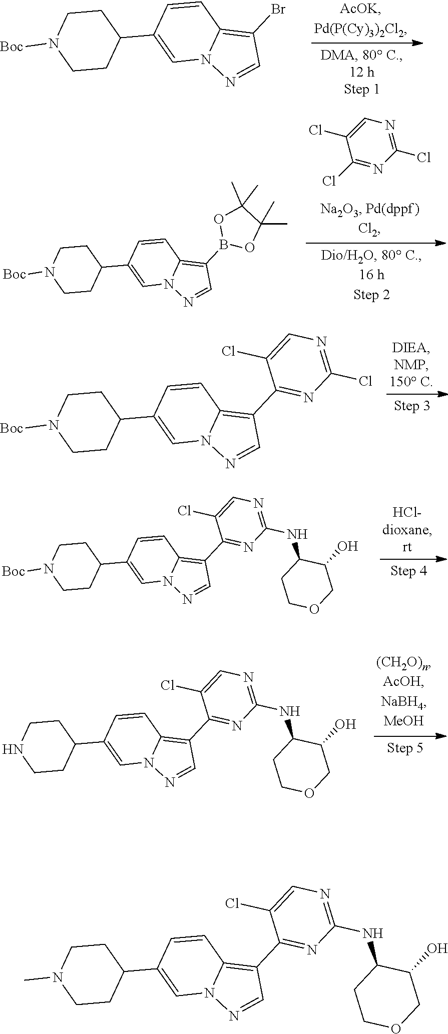

### tert-butyl 4-(3-(4,4,5,5-tetramethyl-1,3,2-dioxaborolan-2-yl)pyrazolo[1,5-a]pyridin-6-yl)piperidine-1-carboxylate

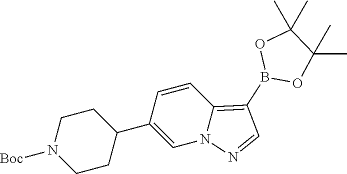 [SMILES: CC(C)(C)OC(=O)N1CCC(c2ccc3c(B4OC(C)(C)C(C)(C)O4)cnn3c2)CC1]

Step 1. To a stirred solution of tert-butyl 4-(3-bromopyrazolo[1,5-a]pyridin-6-yl)piperidine-1-carboxylate (500 mg, 1.31 mmol) and 4,4,4′,4′,5,5,5′,5′-octamethyl-2,2′-bi(1,3,2-dioxaborolane) (668 mg, 2.63 mmol) in DMA (10 mL) was added KOAc (387 mg, 3.94 mmol) and dichloro(tricyclohexylphosphine)palladium (II) (97.1 mg, 0.13 mmol) under N₂. The reaction was stirred at 80° C. for 12 h. The reaction mixture was diluted with water, and the aqueous phase was extracted with EA (30×3 mL). The combined organic layers were washed with brine, dried over sodium sulfate, filtered, and concentrated in vacuo. The residue was purified by reverse flash chromatography with the following conditions: column, C18 silica gel; mobile phase, MeCN in water, 60% to 100% gradient in 10 min; detector, UV 254 nm to afford tert-butyl 4-(3-(4,4,5,5-tetramethyl-1,3,2-dioxaborolan-2-yl)pyrazolo[1,5-a]pyridin-6-yl)piperidine-1-carboxylate (250 mg, 44.5%) as a white solid. m/z (ES⁺) [M+H]⁺=428.40; HPLC tR=1.030 min.

### tert-butyl 4-(3-(2,5-dichloropyrimidin-4-yl)pyrazolo[1,5-a]pyridin-6-yl)piperidine-1-carboxylate

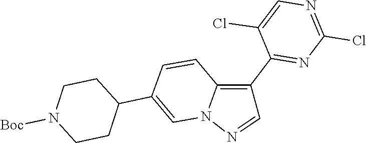 [SMILES: CC(C)(C)OC(=O)N1CCC(c2ccc3c(-c4nc(Cl)ncc4Cl)cnn3c2)CC1]

Step 2. To a stirred solution of tert-butyl 4-(3-(4,4,5,5-tetramethyl-1,3,2-dioxaborolan-2-yl)pyrazolo[1,5-a]pyridin-6-yl)piperidine-1-carboxylate (250 mg, 0.59 mmol) and 2,4,5-trichloropyrimidine (215 mg, 1.17 mmol) in 1,4-Dioxane (10 mL) and H₂O (1 mL) was added Na₂CO₃ (186 mg, 1.76 mmol) and PdCl₂(dppf)-CH₂Cl₂adduct (47.8 mg, 0.06 mmol) under N₂. The reaction was stirred at 80° C. for 16 hours. The reaction mixture was diluted with water, and the aqueous phase was extracted with EA (50×3 mL). The combined organic layers were washed with brine, dried over sodium sulfate, filtered, and concentrated in vacuo. The residue was purified by reverse flash chromatography with the following conditions: column, C18 silica gel; mobile phase, MeCN in water, 60% to 100% gradient in 20 min; detector, UV 254 nm to afford tert-butyl 4-(3-(2,5-dichloropyrimidin-4-yl)pyrazolo[1,5-a]pyridin-6-yl)piperidine-1-carboxylate (200 mg, 76.3%) as a white solid. m/z (ES⁺) [M+H]⁺=448.15; HPLC tR=1.447 min.

### tert-butyl 4-(3-(5-chloro-2-(((3S,4R)-3-hydroxytetrahydro-2H-pyran-4-yl)amino)pyrimidin-4-yl)pyrazolo[1,5-a]pyridin-6-yl)piperidine-1-carboxylate

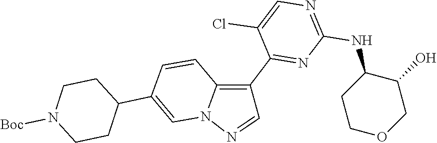 [SMILES: CC(C)(C)OC(=O)N1CCC(c2ccc3c(-c4nc(N[C@@H]5CCOC[C@H]5O)ncc4Cl)cnn3c2)CC1]

Step 3. To a stirred solution of tert-butyl 4-(3-(2,5-dichloropyrimidin-4-yl)pyrazolo[1,5-a]pyridin-6-yl)piperidine-1-carboxylate (195 mg, 0.44 mmol) in NMP (2 mL) were added DIEA (281 mg, 2.17 mmol) and (3S,4R)-4-aminotetrahydro-2H-pyran-3-ol hydrochloride (200 mg, 1.30 mmol). The reaction was stirred at 150° C. for 1 hour. The mixture was purified by reverse flash chromatography with the following conditions: column, C18 silica gel; mobile phase, MeCN in water, 60% to 100% gradient in 20 min; detector, UV 254 nm to afford tert-butyl 4-(3-(5-chloro-2-(((3S,4R)-3-hydroxytetrahydro-2H-pyran-4-yl)amino)pyrimidin-4-yl)pyrazolo[1,5-a]pyridin-6-yl)piperidine-1-carboxylate (100 mg, 43.5%) as a white solid. m/z (ES⁺) [M+H]⁺=529.15; HPLC tR=1.625 min.

### (3S,4R)-4-((5-chloro-4-(6-(piperidin-4-yl)pyrazolo[1,5-a]pyridin-3-yl)pyrimidin-2-yl)amino)tetrahydro-2H-pyran-3-ol

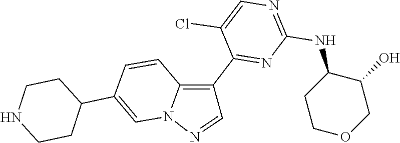 [SMILES: O[C@@H]1COCC[C@H]1Nc1ncc(Cl)c(-c2cnn3cc(C4CCNCC4)ccc23)n1]

Step 4. The tert-butyl 4-(3-(5-chloro-2-(((3S,4R)-3-hydroxytetrahydro-2H-pyran-4-yl)amino)pyrimidin-4-yl)pyrazolo[1,5-a]pyridin-6-yl)piperidine-1-carboxylate (80 mg, 0.15 mmol) in MeOH (2 mL) was added HCl-dioxane (2 mL, 4 molar). The reaction was stirred at 25° C. for 0.5 hour. The mixture was concentrated and used for the next step without any purification. m/z (ES⁺) [M+H]⁺=429.25; HPLC tR=0.822 min.

### (3S,4R)-4-((5-chloro-4-(6-(1-methylpiperidin-4-yl)pyrazolo[1,5-a]pyridin-3-yl)pyrimidin-2-yl)amino)tetrahydro-2H-pyran-3-ol

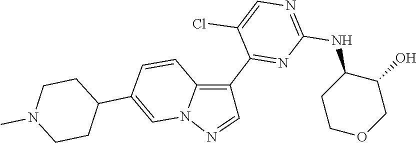 [SMILES: CN1CCC(c2ccc3c(-c4nc(N[C@@H]5CCOC[C@H]5O)ncc4Cl)cnn3c2)CC1]

Step 5. To a stirred solution of (3S,4R)-4-((5-chloro-4-(6-(piperidin-4-yl)pyrazolo[1,5-a]pyridin-3-yl)pyrimidin-2-yl)amino)tetrahydro-2H-pyran-3-ol (60 mg, 0.14 mmol) in MeOH (3 mL) was added AcOH (8.4 mg, 0.14 mmol) and Paraformaldehyde (13 mg, 0.42 mmol) under N₂. The reaction was stirred at 25° C. for 0.5 hour. To the reaction was added NaBH₄ (26 mg, 0.70 mmol) under N₂. The reaction was stirred at 25° C. for 16 hours. The mixture was quenched with water and extracted with EA (3×20 ml) three times. The combined organic layers were washed with brine, dried over sodium sulfate and concentrated. The residue was purified by Prep-HPLC (Column: XBridge Prep OBD C18 Column, 30×150 mm, 5 μm; Mobile Phase A: Water (00 mmol/L NH₄HCO₃+0.05% NH₃H₂O), Mobile Phase B: ACN; Flow rate: 60 mL/min mL/min; Gradient: 20% B to 40% B in 7 min; Wavelength: 220 nm; RT1 (min): 7.82). Lyophilization yielded (3S,4R)-4-((5-chloro-4-(6-(1-methylpiperidin-4-yl)pyrazolo[1,5-a]pyridin-3-yl)pyrimidin-2-yl)amino)tetrahydro-2H-pyran-3-ol (3.8 mg, 6.0%) as an off-white amorphous solid. m/z (ES⁺) [M+H]⁺=443.20; HPLC tR=1.158 min. ¹H NMR (400 MHz, DMSO-d6) δ 8.88 (s, 1H), 8.68 (s, 2H), 8.29 (s, 1H), 7.44 (d, J=84.4 Hz, 2H), 5.02 (s, 1H), 3.94-3.78 (m, 3H), 3.52 (td, J=9.4, 4.8 Hz, 1H), 3.39 (s, 1H), 3.09 (s, 1H), 2.89 (dt, J=11.4, 3.2 Hz, 2H), 2.68-2.56 (m, 1H), 2.21 (s, 3H), 1.98 (td, J=11.5, 2.7 Hz, 3H), 1.87-1.69 (m, 4H), 1.50 (qd, J=11.9, 4.5 Hz, 1H).

### Example 45

### (3S,4R)-4-((5-chloro-4-(6-(1-methylazetidin-3-yl)pyrazolo[1,5-a]pyridin-3-yl)pyrimidin-2-yl)amino)tetrahydro-2H-pyran-3-ol (I-977)

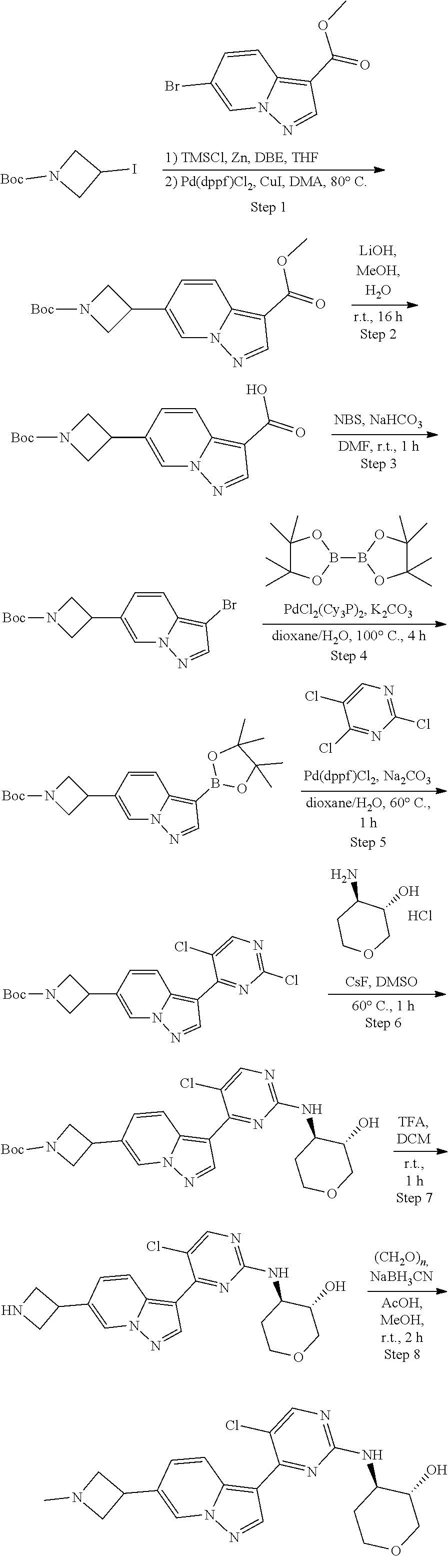

### methyl 6-(1-(tert-butoxycarbonyl)azetidin-3-yl)pyrazolo[1,5-a]pyridine-3-carboxylate

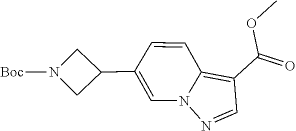 [SMILES: COC(=O)c1cnn2cc(C3CN(C(=O)OC(C)(C)C)C3)ccc12]

Step 1. A round bottom flask was charged with zinc (4.2 g, 3.3 Eq, 64 mmol), DMA (10 mL) and a stirbar before being evacuated and purged with nitrogen three times. 1,2-dibromoethane (0.36 g, 0.1 Eq, 1.9 mmol) and TMS-C₁ (0.21 g, 0.24 mL, 0.1 Eq, 1.9 mmol) was added. The mixture was stirred at 20° C. for 30 min. Tert-butyl 3-iodoazetidine-1-carboxylate (12 g, 2.2 Eq, 42 mmol) in DMA (10 mL) was added dropwise. The mixture was stirred at 20° C. for 2 hours to result in the Zn reagent. A resealable reaction vial was charged with methyl 6-bromopyrazolo[1,5-a]pyridine-3-carboxylate (5 g, 1 Eq, 0.02 mol), Pd (dppf) Cl₂ (1.4 g, 0.1 Eq, 1.9 mmol), CuI (0.37 g, 0.1 Eq, 1.9 mmol), DMA (10 mL) and a stirbar before being evacuated and purged with nitrogen three times. The Zn-reagent was filtered and the filtrate was added into the above solution. The mixture was stirred at 80° C. for 2 hours. The mixture was diluted with water (200 mL), and the aqueous phase was extracted with EA (50 mL) three times. The combined organic layers were washed with brine, dried over sodium sulfate, filtered, concentrated in vacuo. The residue was purified by Flash (acetonitrile/water). Concentration in vacuo yielded methyl 6-(1-(tert-butoxycarbonyl)azetidin-3-yl)pyrazolo[1,5-a]pyridine-3-carboxylate (5.9 g, 18 mmol, 92%) as a yellow oil. m/z (ES⁺) [M+H]⁺=332.05; HPLC tR=0.921 min.

### 6-(1-(tert-butoxycarbonyl)azetidin-3-yl)pyrazolo[1,5-a]pyridine-3-carboxylic acid

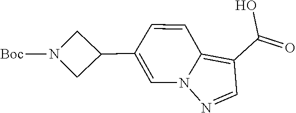 [SMILES: CC(C)(C)OC(=O)N1CC(c2ccc3c(C(=O)O)cnn3c2)C1]

Step 2. A round bottom flask was charged with methyl 6-(1-(tert-butoxycarbonyl)azetidin-3-yl)pyrazolo[1,5-a]pyridine-3-carboxylate (5.9 g, 1 Eq, 18 mmol), MeOH (200 mL) and a stirbar. LiOH (3.4 g, 8 Eq, 0.14 mol) was dissolved in Water (100 mL) and added into the mixture. The solution was stirred at 20° C. for 16 hours. Concentration in vacuo. The reaction mixture was adjusted the pH value to 5-6 with 1M hydrochloric acid. The mixture was extracted with EA (100 mL) three times. The combined organic layers were washed with brine, dried over sodium sulfate, filtered, and concentrated in vacuo. This yielded 6-(1-(tert-butoxycarbonyl)azetidin-3-yl)pyrazolo[1,5-a]pyridine-3-carboxylic acid (5.1 g, 16 mmol, 90%) as a yellow solid. m/z (ES⁺) [M+H]⁺=318.05; HPLC tR=0.521 min.

### tert-butyl 3-(3-bromopyrazolo[1,5-a]pyridin-6-yl)azetidine-1-carboxylate

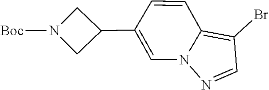 [SMILES: CC(C)(C)OC(=O)N1CC(c2ccc3c(Br)cnn3c2)C1]

Step 3. A resealable reaction vial was charged with 6-(1-(tert-butoxycarbonyl)azetidin-3-yl)pyrazolo[1,5-a]pyridine-3-carboxylic acid (5.1 g, 1 Eq, 16 mmol), NaHCO₃ (4.1 g, 3 Eq, 48 mmol), DMF (100 mL) and a stirbar. NBS (3.1 g, 1.1 Eq, 18 mmol) was added in portions at 0° C. The solution was stirred at 20° C. for 1 hour. The mixture was diluted with water (500 mL), and the aqueous phase was extracted with EA (200 mL) three times. The combined organic layers were washed with brine, dried over sodium sulfate, filtered, and concentrated in vacuo. This resulted in tert-butyl 3-(3-bromopyrazolo[1,5-a]pyridin-6-yl)azetidine-1-carboxylate (5.8 g, 16 mmol, 98%, 96% Purity) as a yellow solid. m/z (ES⁺) [M+H+ACN]⁻=392.95; HPLC tR=0.783 min.

### tert-butyl 3-(3-(4,4,5,5-tetramethyl-1,3,2-dioxaborolan-2-yl)pyrazolo[1,5-a]pyridin-6-yl)azetidine-1-carboxylate

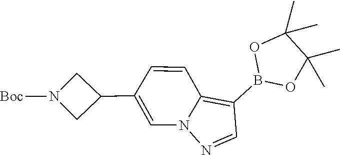 [SMILES: CC(C)(C)OC(=O)N1CC(c2ccc3c(B4OC(C)(C)C(C)(C)O4)cnn3c2)C1]

Step 4. A resealable reaction vial was charged with tert-butyl 3-(3-bromopyrazolo[1,5-a]pyridin-6-yl)azetidine-1-carboxylate (1 g, 1 Eq, 3 mmol), 4,4,4′,4′,5,5,5′,5′-octamethyl-2,2′-bi(1,3,2-dioxaborolane) (1 g, 2 Eq, 6 mmol), K₂CO₃ (1 g, 3 Eq, 9 mmol), Dichloro(tricyclohexylphosphine)palladium (II) (0.4 g, 0.2 Eq, 0.6 mmol), 1,4-Dioxane (20 mL) and a stirbar before being evacuated and purged with nitrogen three times. The mixture was stirred at 100° C. for 4 hours. Concentration in vacuo. The resulting crude material was purified by Flash (acetonitrile/water). Concentration in vacuo yielded tert-butyl 3-(3-(4,4,5,5-tetramethyl-1,3,2-dioxaborolan-2-yl)pyrazolo[1,5-a]pyridin-6-yl)azetidine-1-carboxylate (600 mg, 1.50 mmol, 50%) as a yellow solid. m/z (ES⁺) [M+H]⁺=400.15; HPLC tR=1.101 min.

### tert-butyl 3-(3-(2,5-dichloropyrimidin-4-yl)pyrazolo[1,5-a]pyridin-6-yl)azetidine-1-carboxylate

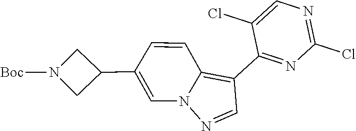 [SMILES: CC(C)(C)OC(=O)N1CC(c2ccc3c(-c4nc(Cl)ncc4Cl)cnn3c2)C1]

Step 5. A resealable reaction vial was charged with tert-butyl 3-(3-(4,4,5,5-tetramethyl-1,3,2-dioxaborolan-2-yl)pyrazolo[1,5-a]pyridin-6-yl)azetidine-1-carboxylate (500 mg, 1 Eq, 1.25 mmol), 2,4,5-trichloropyrimidine (276 mg, 1.2 Eq, 1.50 mmol), Na₂CO₃ (398 mg, 3 Eq, 3.76 mmol), Pd(dppf)Cl₂ (183 mg, 0.2 Eq, 250 mol), 1,4-Dioxane (4 mL), water (1 mL) and a stirbar before being evacuated and purged with nitrogen three times. The mixture was stirred at 60° C. for 1 hour. The resulting crude material was purified by Flash (acetonitrile/water). Concentration in vacuo yielded tert-butyl 3-(3-(2,5-dichloropyrimidin-4-yl)pyrazolo[1,5-a]pyridin-6-yl)azetidine-1-carboxylate (240 mg, 0.37 mmol, 29%, 64% Purity) as a yellow solid. m/z (ES⁺) [M+H]⁺=419.95; HPLC tR=0.789 min.

### tert-butyl 3-(3-(5-chloro-2-(((3S,4R)-3-hydroxytetrahydro-2H-pyran-4-yl)amino)pyrimidin-4-yl)pyrazolo[1,5-a]pyridin-6-yl)azetidine-1-carboxylate

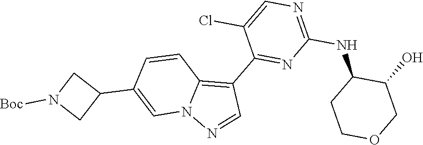 [SMILES: CC(C)(C)OC(=O)N1CC(c2ccc3c(-c4nc(N[C@@H]5CCOC[C@H]5O)ncc4Cl)cnn3c2)C1]

Step 6. A resealable reaction vial was charged with tert-butyl 3-(3-(2,5-dichloropyrimidin-4-yl)pyrazolo[1,5-a]pyridin-6-yl)azetidine-1-carboxylate (230 mg, 64% Wt, 1 Eq, 350 μmol), (3S,4R)-4-aminotetrahydro-2H-pyran-3-ol hydrochloride (80.7 mg, 1.5 Eq, 525 μmol), DMSO (8 mL) and a stirbar. The mixture was stirred at 60° C. for 1 hour. The mixture was diluted with water (50 mL), and the aqueous phase was extracted with EA (10 mL) three times. The combined organic layers were washed with brine, dried over sodium sulfate, filtered, and concentrated in vacuo. The resulting crude material was purified by Flash (acetonitrile/water). Concentration in vacuo yielded tert-butyl 3-(3-(5-chloro-2-(((3S,4R)-3-hydroxytetrahydro-2H-pyran-4-yl)amino)pyrimidin-4-yl)pyrazolo[1,5-a]pyridin-6-yl)azetidine-1-carboxylate (100 mg, 200 mol, 57.0%) as a yellow solid. m/z (ES⁺) [M+H]⁺=501.15; HPLC tR=0.961 min.

### (3S,4R)-4-((4-(6-(azetidin-3-yl)pyrazolo[1,5-a]pyridin-3-yl)-5-chloropyrimidin-2-yl)amino)tetrahydro-2H-pyran-3-ol

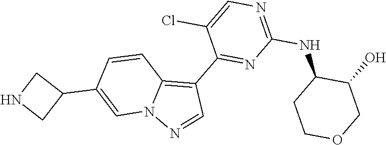 [SMILES: O[C@@H]1COCC[C@H]1Nc1ncc(Cl)c(-c2cnn3cc(C4CNC4)ccc23)n1]

Step 7. A resealable reaction vial was charged with tert-butyl 3-(3-(5-chloro-2-(((3S,4R)-3-hydroxytetrahydro-2H-pyran-4-yl)amino)pyrimidin-4-yl)pyrazolo[1,5-a]pyridin-6-yl)azetidine-1-carboxylate (95 mg, 1 Eq, 0.19 mmol), DCM (2 mL) and a stirbar. TFA (1 mL) in DCM (2 mL) was added dropwise into the above solution at 0° C. The mixture was stirred at 20° C. for 1 hour. Concentration in vacuo. The resulting crude material was purified by Flash (acetonitrile/water (0.01% hydrochloric acid)). Concentration in vacuo yielded (3S,4R)-4-((4-(6-(azetidin-3-yl)pyrazolo[1,5-a]pyridin-3-yl)-5-chloropyrimidin-2-yl)amino)tetrahydro-2H-pyran-3-ol (150 mg, 0.15 mmol, 79%, 40% Purity) as a yellow solid. m/z (ES⁺) [M+H]⁺=401.05; HPLC tR=0.561 min.

### (3S,4R)-4-((5-chloro-4-(6-(1-methylazetidin-3-yl)pyrazolo[1,5-a]pyridin-3-yl)pyrimidin-2-yl)amino)tetrahydro-2H-pyran-3-ol

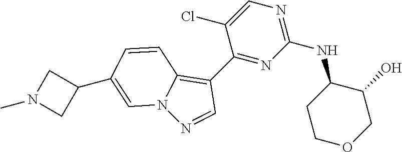 [SMILES: CN1CC(c2ccc3c(-c4nc(N[C@@H]5CCOC[C@H]5O)ncc4Cl)cnn3c2)C1]

Step 8. A resealable reaction vial was charged with (3S,4R)-4-((4-(6-(azetidin-3-yl)pyrazolo[1,5-a]pyridin-3-yl)-5-chloropyrimidin-2-yl)amino)tetrahydro-2H-pyran-3-ol (150 mg, 40% Wt, 1 Eq, 150 μmol), AcOH (1.80 mg, 1.71 μL, 0.2 Eq, 29.9 μmol), paraformaldehyde (89.9 mg, 20 Eq, 2.99 mmol), MeOH (5 mL) and a stirbar. Sodium cyanotrihydroborate (94.1 mg, 10 Eq, 1.50 mmol) was added. The solution was stirred at 25° C. for 2 hours. Concentration in vacuo. The resulting crude material was purified by prep-HPLC (Column: XBridge Prep OBD C18 Column, 30×150 mm, 5 μm; Mobile Phase A: Water (10 mmol/LNH₄HCO₃)+0.05% NH₃·H₂O, Mobile Phase B: ACN; Flow rate: 60 mL/min mL/min; Gradient: 13% B to 40% B in 7 min; Wavelength: 254 nm/220 nm nm; RT1 (min): 7.68). Lyophilization yielded (3S,4R)-4-((5-chloro-4-(6-(1-methylazetidin-3-yl)pyrazolo[1,5-a]pyridin-3-yl)pyrimidin-2-yl)amino)tetrahydro-2H-pyran-3-ol (17.2 mg, 41.5 μmol, 27.7%) as a white solid. m/z (ES⁺) [M+H]⁺=415.10; HPLC tR=0.858 min. ¹H NMR (400 MHz, DMSO-d6) 8.89 (s, 1H), 8.80 (s, 2H), 8.30 (s, 1H), 7.67 (s, 1H), 7.34 (s, 1H), 5.02 (s, 1H), 3.86 (dd, J=10.9, 4.6 Hz, 3H), 3.70 (dd, J=14.1, 7.2 Hz, 1H), 3.60 (t, J=7.1 Hz, 2H), 3.52 (dt, J=9.4, 4.4 Hz, 1H), 3.40 (s, 1H), 3.20 (t, J=6.4 Hz, 2H), 3.11 (s, 1H), 2.30 (s, 3H), 2.00 (d, J=13.1 Hz, 1H), 1.60-1.43 (m, 1H).

### Example 46

### (3S,4R)-4-((5-chloro-4-(7-methyl-6-(1-methylpiperidin-4-yl)pyrazolo[1,5-a]pyridin-3-yl)pyrimidin-2-yl)amino)tetrahydro-2H-pyran-3-ol (I-980)

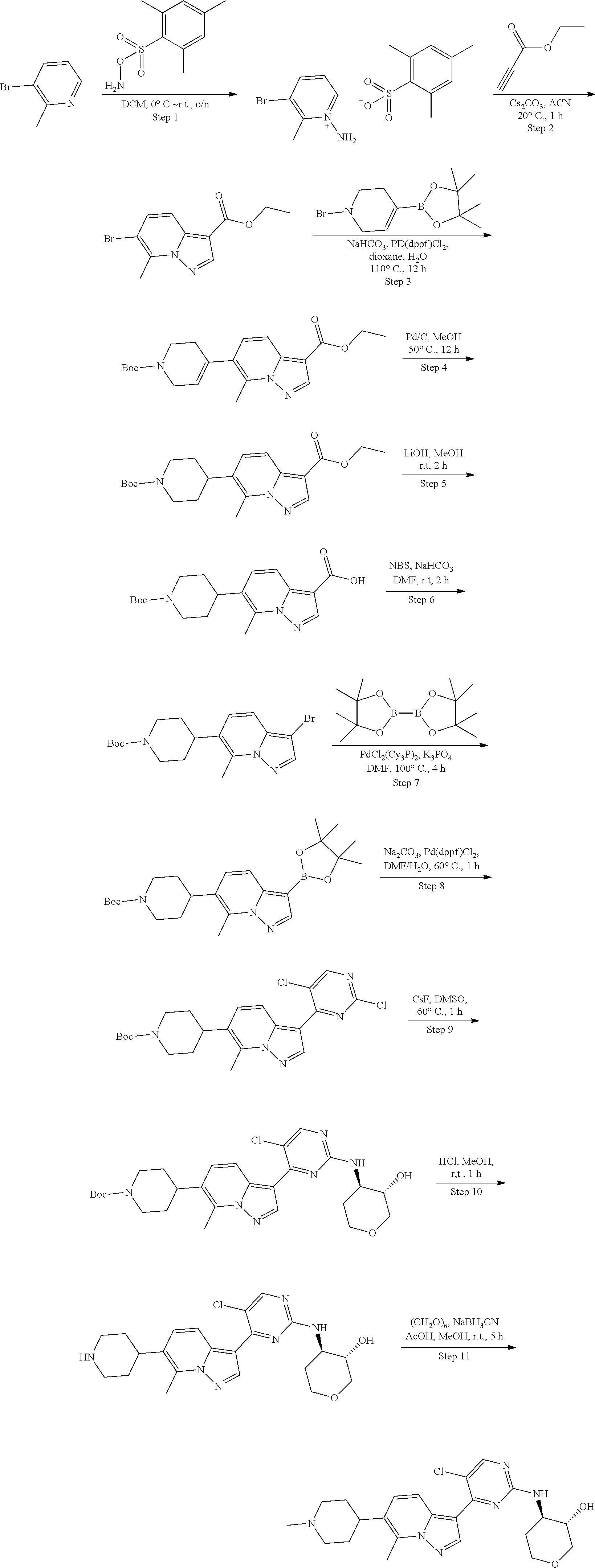

### 1-amino-3-bromopyridin-1-ium 2,4,6-trimethylbenzenesulfonate

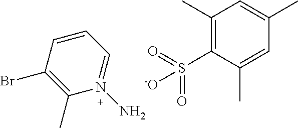

Step 1. A mixture of 3-bromo-2-methylpyridine (4.000 g, 1 Eq, 23.25 mmol) and O-(mesitylsulfonyl) hydroxylamine (5.005 g, 1 Eq, 23.25 mmol) in DCM (80 mL) was stirred for 10 mins at 0° C. under N₂ atmosphere. The reaction is stirred at RT for 2 hours. Desired product could be detected by LCMS. The resulting mixture was concentrated under vacuum. This resulted 1-amino-3-bromopyridin-1-ium 2,4,6-trimethylbenzenesulfonate as a crude white amorphous solid (9.0 g, crude). m/z (ES⁺) [M+H]⁺=186.95; HPLC tR=0.538 min.

### ethyl 6-bromo-7-methylpyrazolo[1,5-a]pyridine-3-carboxylate

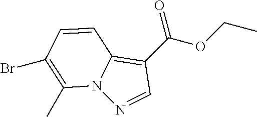 [SMILES: CCOC(=O)c1cnn2c(C)c(Br)ccc12]

Step 2. A mixture of 1-amino-3-bromopyridin-1-ium 2,4,6-trimethylbenzenesulfonate (9 g, 1 Eq, 0.05 mol), ethyl propiolate (7 g, 1.5 Eq, 0.07 mol) and Cs₂CO₃ (0.05 kg, 3 Eq, 0.1 mol) in ACN (150 mL) was stirred for 1 hour at 20° C. Desired product could be detected by LCMS. The resulting mixture was concentrated under vacuum. The residue was purified by reversed-phase flash chromatography with the following conditions: column, C18 silica gel; mobile phase, ACN in water, 30% to 80% gradient in 20 min; detector, UV 254 nm. This resulted in ethyl 6-bromo-7-methylpyrazolo[1,5-a]pyridine-3-carboxylate (1.7 g, 30%) as a yellow oil. m/z (ES⁺) [M+H]⁺=283.00; HPLC tR=1.138 min.

### ethyl 6-(1-(tert-butoxycarbonyl)-1,2,3,6-tetrahydropyridin-4-yl)-7-methylpyrazolo[1,5-a]pyridine-3-carboxylate

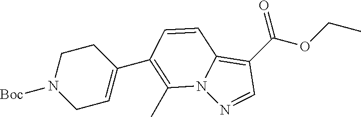 [SMILES: CCOC(=O)c1cnn2c(C)c(C3=CCN(C(=O)OC(C)(C)C)CC3)ccc12]

Step 3. A mixture of ethyl 6-bromo-7-methylpyrazolo[1,5-a]pyridine-3-carboxylate (1.7 g, 1 Eq, 6.0 mmol), tert-butyl 4-(4,4,5,5-tetramethyl-1,3,2-dioxaborolan-2-yl)-3,6-dihydropyridine-1(2H)-carboxylate (2.8 g, 1.5 Eq, 9.0 mmol), NaHCO₃ (1.5 g, 3 Eq, 18 mmol) and Pd(dppf)Cl₂ (0.50 g, 0.1 Eq, 0.60 mmol) in 1,4-dioxane (30 mL) and water (6 mL) was stirred for 12 hours at 110° C. under N₂ atmosphere. Desired product could be detected by LCMS. The resulting mixture was concentrated under vacuum. The residue was purified by reversed-phase flash chromatography with the following conditions: column, C18 silica gel; mobile phase, ACN in water, 30% to 70% gradient in 20 min; detector, UV 254 nm. This resulted in ethyl 6-(1-(tert-butoxycarbonyl)-1,2,3,6-tetrahydropyridin-4-yl)-7-methylpyrazolo[1,5-a]pyridine-3-carboxylate (1.6 g, 65%) as a yellow oil. m/z (ES⁺) [M+H]⁺=386.30; HPLC tR=0.929 min.

### ethyl 6-(1-(tert-butoxycarbonyl)piperidin-4-yl)-7-methylpyrazolo[1,5-a]pyridine-3-carboxylate

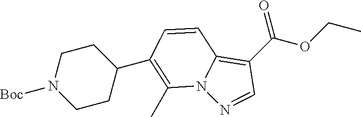 [SMILES: CCOC(=O)c1cnn2c(C)c(C3CCN(C(=O)OC(C)(C)C)CC3)ccc12]

Step 4. A mixture of ethyl 6-(1-(tert-butoxycarbonyl)-1,2,3,6-tetrahydropyridin-4-yl)-7-methylpyrazolo[1,5-a]pyridine-3-carboxylate (1.5 g, 1 Eq, 3.9 mmol) and Pd/C (0.69 g, 1.5 Eq, 5.8 mmol) in MeOH (10 mL) was stirred for 12 hours at 50° C. under H₂ atmosphere. Desired product could be detected by LCMS. The resulting mixture was filtered, the filter cake was washed with MeOH (3×50 mL). The filtrate was concentrated under reduced pressure. This resulted in ethyl 6-(1-(tert-butoxycarbonyl)piperidin-4-yl)-7-methylpyrazolo[1,5-a]pyridine-3-carboxylate (1.4 g, 95%) as a yellow oil. m/z (ES⁺) [M+H]⁺=388.25; HPLC tR=0.808 min.

### 6-(1-(tert-butoxycarbonyl) piperidin-4-yl)-7-methylpyrazolo[1,5-a]pyridine-3-carboxylic acid

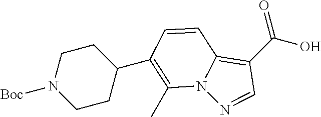 [SMILES: Cc1c(C2CCN(C(=O)OC(C)(C)C)CC2)ccc2c(C(=O)O)cnn12]

Step 5. A mixture of ethyl 6-(1-(tert-butoxycarbonyl)piperidin-4-yl)-7-methylpyrazolo[1,5-a]pyridine-3-carboxylate (1.3 g, 1 Eq, 3.4 mmol) in MeOH (20 mL), and treated with potassium hydroxide (0.56 g, 3 Eq, 10 mmol) in water (10 mL). The mixture is stirred at 20° C. for 2 hours. Desired product could be detected by LCMS. The resulting mixture was concentrated under vacuum. The reaction mixture was diluted with H₂O (100 mL), then adjusted to pH 5-6 with 1M HCl and the aqueous phase was extracted with EA (100 mL) three times. The combined organic layers were washed with brine, dried over sodium sulfate, filtered and concentrated in vacuo. This resulted in 6-(1-(tert-butoxycarbonyl) piperidin-4-yl)-7-methylpyrazolo[1,5-a]pyridine-3-carboxylic acid (1.2 g, 95%) as a yellow oil. m/z (ES⁺) [M+ACN]⁺=401.05; HPLC tR=0.708 min.

### tert-butyl 4-(3-bromo-7-methylpyrazolo[1,5-a]pyridin-6-yl)piperidine-1-carboxylate

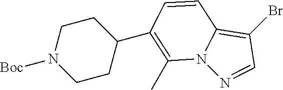 [SMILES: Cc1c(C2CCN(C(=O)OC(C)(C)C)CC2)ccc2c(Br)cnn12]

Step 6. A mixture of 6-(1-(tert-butoxycarbonyl)piperidin-4-yl)-7-methylpyrazolo[1,5-a]pyridine-3-carboxylic acid (1.2 g, 1 Eq, 3.3 mmol), NaHCO₃ (0.84 g, 3 Eq, 10 mmol) and N-(12-boraneylidene)thiohydroxylamine (0.23 g, 1.2 Eq, 4.0 mmol) in DMF (20 mL) was stirred for 2 hours at 20° C. atmosphere. Desired product could be detected by LCMS. The reaction mixture was diluted with H₂O (100 mL), and the aqueous phase was extracted with EA (300 mL) three times. The combined organic layers were washed with brine, dried over sodium sulfate, filtered and concentrated in vacuo. After filtration, the filtrate was concentrated under reduced pressure. The resulting mixture was concentrated under vacuum. The residue was purified by reversed-phase flash chromatography with the following conditions: column, C18 silica gel; mobile phase, ACN in water, 30% to 80% gradient in 20 min; detector, UV 254 nm. This resulted in tert-butyl 4-(3-bromo-7-methylpyrazolo[1,5-a]pyridin-6-yl)piperidine-1-carboxylate (300 mg, 23%) as a yellow oil. m/z (ES⁺) [M+H]⁺=394.05; HPLC tR=0.998 min.

### tert-butyl tert-butyl 4-(7-methyl-3-(4,4,5,5-tetramethyl-1,3,2-dioxaborolan-2-yl)pyrazolo[1,5-a]pyridin-6-yl)piperidine-1-carboxylate

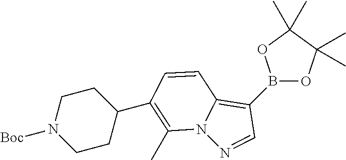 [SMILES: Cc1c(C2CCN(C(=O)OC(C)(C)C)CC2)ccc2c(B3OC(C)(C)C(C)(C)O3)cnn12]

Step 7. A mixture of tert-butyl 4-(3-bromo-7-methylpyrazolo[1,5-a]pyridin-6-yl)piperidine-1-carboxylate (200 mg, 1 Eq, 507 μmol) and 4,4,4′,4′,5,5,5′,5′-octamethyl-2,2′-bi(1,3,2-dioxaborolane) (167 mg, 1.3 Eq, 659 mol), K₃PO₄ (323 mg, 3 Eq, 1.52 mmol), PdCl₂(Cy₃P)₂ (120 mg, 0.3 Eq, 152 μmol) in DMF (3 mL) was stirred for 4 hours at 100° C. under N₂ atmosphere. Desired product could be detected by LCMS. The reaction mixture was diluted with H₂O (30 mL), and the aqueous phase was extracted with EA (30 mL) three times. The combined organic layers were washed with brine, dried over sodium sulfate, filtered and concentrated in vacuo. After filtration, the filtrate was concentrated under reduced pressure. The resulting mixture was concentrated under vacuum. The residue was purified by reversed-phase flash chromatography with the following conditions: column, C18 silica gel; mobile phase, ACN in water, 30% to 80% gradient in 20 min; detector, UV254 nm. This resulted in tert-butyl tert-butyl 4-(7-methyl-3-(4,4,5,5-tetramethyl-1,3,2-dioxaborolan-2-yl)pyrazolo[1,5-a]pyridin-6-yl)piperidine-1-carboxylate (100 mg, 36%) as a yellow oil. m/z (ES⁺) [M+H]⁺=442.25; HPLC tR=1.298 min.

### tert-butyl 4-(3-(2,5-dichloropyrimidin-4-yl)-7-methylpyrazolo[1,5-a]pyridin-6-yl)piperidine-1-carboxylate

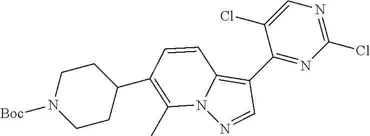 [SMILES: Cc1c(C2CCN(C(=O)OC(C)(C)C)CC2)ccc2c(-c3nc(Cl)ncc3Cl)cnn12]

Step 8. A mixture of tert-butyl 4-(7-methyl-3-(4,4,5,5-tetramethyl-1,3,2-dioxaborolan-2-yl)pyrazolo[1,5-a]pyridin-6-yl)piperidine-1-carboxylate (100 mg, 1 Eq, 227 μmol), 2,4,5-trichloropyrimidine (62.3 mg, 1.5 Eq, 340 mol), Pd(dppf)Cl₂ (16.9 mg, 0.1 Eq, 22.7 μmol) and Na₂CO₃ (72.0 mg, 3 Eq, 680 mol) in DMF (3 mL) and water (0.6 mL) was stirred for 1 hour at 60° C. atmosphere. Desired product could be detected by LCMS. The resulting mixture was concentrated under vacuum. The residue was purified by reversed-phase flash chromatography with the following conditions: column, C18 silica gel; mobile phase, ACN in water, 40% to 80% gradient in 20 min; detector, UV 254 nm. This resulted in tert-butyl 4-(3-(2,5-dichloropyrimidin-4-yl)-7-methylpyrazolo[1,5-a]pyridin-6-yl)piperidine-1-carboxylate (65 mg, 50%) as a white solid. m/z (ES⁺) [M+H]⁻=462.00; HPLC tR=0.942 min.

### tert-butyl 4-(3-(5-chloro-2-(((3S,4R)-3-hydroxytetrahydro-2H-pyran-4-yl)amino)pyrimidin-4-yl)-7-methylpyrazolo[1,5-a]pyridin-6-yl)piperidine-1-carboxylate

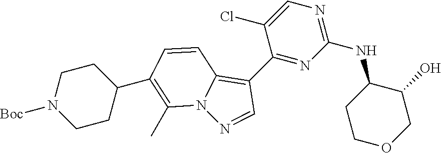 [SMILES: Cc1c(C2CCN(C(=O)OC(C)(C)C)CC2)ccc2c(-c3nc(N[C@@H]4CCOC[C@H]4O)ncc3Cl)cnn12]

Step 9. A mixture of tert-butyl 4-(3-(2,5-dichloropyrimidin-4-yl)-7-methylpyrazolo[1,5-a]pyridin-6-yl)piperidine-1-carboxylate (65 mg, 1 Eq, 0.14 mmol), (3S,4R)-4-aminotetrahydro-2H-pyran-3-ol (25 mg, 1.5 Eq, 0.21 mmol) and CsF (0.11 g, 5 Eq, 0.70 mmol) in DMSO (3 mL) was stirred for 1 hour at 60° C. atmosphere. Desired product could be detected by LCMS. The resulting mixture was concentrated under vacuum. The residue was purified by reversed-phase flash chromatography with the following conditions: column, C18 silica gel; mobile phase, ACN in water, 40% to 80% gradient in 20 min; detector, UV 254 nm. This resulted in tert-butyl 4-(3-(5-chloro-2-(((3S,4R)-3-hydroxytetrahydro-2H-pyran-4-yl)amino)pyrimidin-4-yl)-7-methylpyrazolo[1,5-a]pyridin-6-yl)piperidine-1-carboxylate (55 mg, 72%) as a colorless oil.

### (3S,4R)-4-((5-chloro-4-(7-methyl-6-(piperidin-4-yl) pyrazolo[1,5-a]pyridin-3-yl) pyrimidin-2-yl) amino) tetrahydro-2H-pyran-3-ol

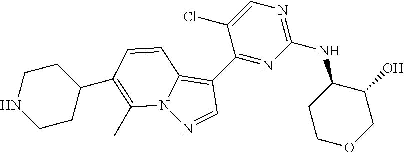 [SMILES: Cc1c(C2CCNCC2)ccc2c(-c3nc(N[C@@H]4CCOC[C@H]4O)ncc3Cl)cnn12]

Step 10. A mixture of tert-butyl 4-(3-(5-chloro-2-(((3S,4R)-3-hydroxytetrahydro-2H-pyran-4-yl) amino) pyrimidin-4-yl)-7-methylpyrazolo[1,5-a]pyridin-6-yl) piperidine-1-carboxylate (50 mg, 1 Eq, 92 μmol) and HCl (2M in 1,4-Dioxane, 0.5 mL) in MeOH (2 mL) was stirred for 1 hour at 20° C. atmosphere. Desired product could be detected by LCMS. The resulting mixture was concentrated under vacuum. This resulted in (3S,4R)-4-((5-chloro-4-(7-methyl-6-(piperidin-4-yl) pyrazolo[1,5-a]pyridin-3-yl) pyrimidin-2-yl) amino) tetrahydro-2H-pyran-3-ol (40 mg, 95%) as a white solid. m/z (ES⁺) [M+H]⁺=443.05; HPLC tR=0.542 min.

### (3S,4R)-4-((5-chloro-4-(7-methyl-6-(1-methylpiperidin-4-yl)pyrazolo[1,5-a]pyridin-3-yl)pyrimidin-2-yl)amino)tetrahydro-2H-pyran-3-ol

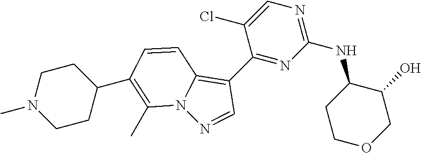 [SMILES: Cc1c(C2CCN(C)CC2)ccc2c(-c3nc(N[C@@H]4CCOC[C@H]4O)ncc3Cl)cnn12]

Step 11. A mixture of (3S,4R)-4-((5-chloro-4-(7-methyl-6-(piperidin-4-yl)pyrazolo[1,5-a]pyridin-3-yl)pyrimidin-2-yl)amino)tetrahydro-2H-pyran-3-ol (30 mg, 1 Eq, 68 μmol), NaBH₃CN (43 mg, 10 Eq, 0.68 mmol), AcOH (1.2 mg, 1.2 μL, 0.3 Eq, 20 μmol) and (CH₂O)~(31 mg, 10 Eq, 0.68 mmol) in MeOH (3 mL) was stirred for 5 hour at 20° C. atmosphere. Desired product could be detected by LCMS. The resulting mixture was concentrated under vacuum. The residue was purified by reversed-phase flash chromatography with the following conditions: column, C18 silica gel; mobile phase, ACN in water, 10% to 50% gradient in 10 min; detector, UV 254 nm. The fractions were concentration. The product was purified by Prep-HPLC: Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 24% B to 50% B in 7 min; Wavelength: 254 nm/220 nm nm; RT1 (min): 7.35. This resulted in (3S,4R)-4-((5-chloro-4-(7-methyl-6-(1-methylpiperidin-4-yl)pyrazolo[1,5-a]pyridin-3-yl)pyrimidin-2-yl)amino)tetrahydro-2H-pyran-3-ol (3.1 mg, 8%) as a white solid. m/z (ES⁺) [M+H]⁺=457.15; HPLC tR=1.302 min. ¹H NMR (400 MHz, DMSO-d6) 8.93 (s, 1H), 8.67 (s, 1H), 8.28 (s, 1H), 7.43 (d, J=92.1 Hz, 2H), 5.01 (d, J=5.3 Hz, 1H), 3.91-3.79 (m, 3H), 3.52 (s, 2H), 3.11 (s, 1H), 2.91 (d, J=11.3 Hz, 3H), 2.80 (s, 3H), 2.23 (s, 3H), 2.11-2.03 (m, 3H), 1.82 (d, J=12.4 Hz, 2H), 1.69 (d, J=12.3 Hz, 2H), 1.50 (qd, J=12.1, 4.6 Hz, 2H).

### Example 47

### (3S,4R)-4-((5-chloro-4-(1-methyl-2-(1-methylpiperidin-4-yl)-1H-imidazol-5-yl) pyrimidin-2-yl) amino) tetrahydro-2H-pyran-3-ol (I-929)

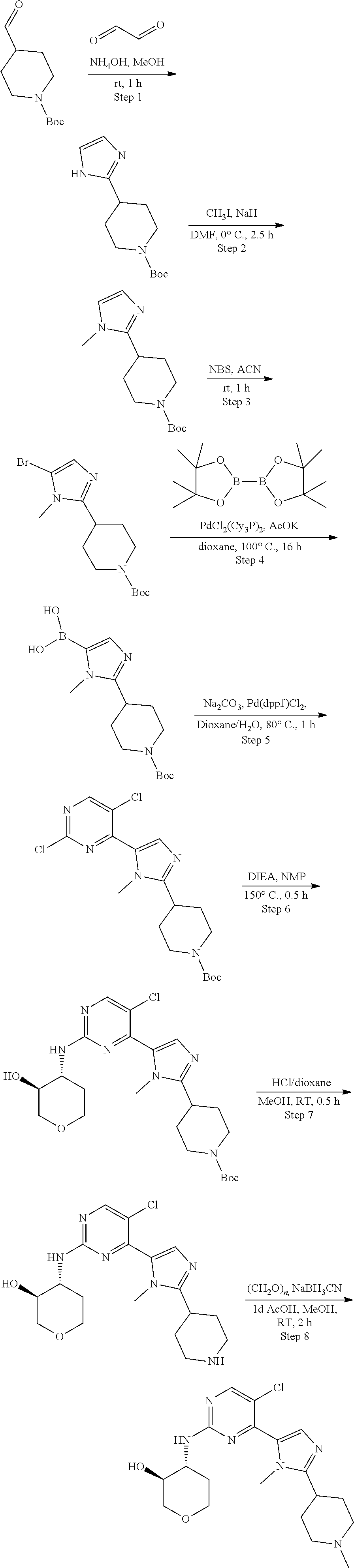

### tert-butyl 4-(1H-imidazol-2-yl) piperidine-1-carboxylate

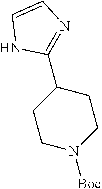 [SMILES: CC(C)(C)OC(=O)N1CCC(c2ncc[nH]2)CC1]

Step 1. A solution of tert-butyl 4-formylpiperidine-1-carboxylate (5.1 g, 24 mmol) in MeOH (10 mL) was treated with Ammonia water (25 mL, 0.19 mol), followed by oxalaldehyde (3 mL, 0.03 mol). The contents were allowed to stir at room temperature for 1 h before it was evaporated to remove methanol. The remains were treated with brine (150 ml) three times and extracted with dichloromethane (50 ml) three times. The organics were dried over sodium sulfate and concentrated to afford tert-butyl 4-(1H-imidazol-2-yl) piperidine-1-carboxylate (5.69 g, 22.6 mmol, 95%) as an off-white solid. m/z (ES⁺) [M+H]⁺=252.20; HPLC tR=0.637 min.

### tert-butyl 4-(1-methyl-1H-imidazol-2-yl) piperidine-1-carboxylate

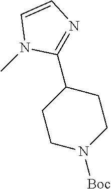 [SMILES: Cn1ccnc1C1CCN(C(=O)OC(C)(C)C)CC1]

Step 2. To an ice cold solution of tert-butyl 4-(1H-imidazol-2-yl) piperidine-1-carboxylate (5.69 g, 22.6 mmol) in DMF (55 mL), NaH (1.18 g, 29.4 mmol) was added. After 30 min, Methyl iodide (3.86 g, 27.2 mmol) was added. The mixture was stirred at 0° C. for 2 h. The mixture was quenched by water and extracted with ethyl acetate (3×200 ml), dried over Na₂SO₄ and evaporated in vacuo. Solvent evaporation afforded tert-butyl 4-(1-methyl-1H-imidazol-2-yl) piperidine-1-carboxylate (5.8 g, 22 mmol, 97%) as an orange oil. m/z (ES⁺) [M+H]⁺=266.20; HPLC tR=0.730 min.

### tert-butyl 4-(5-bromo-1-methyl-1H-imidazol-2-yl) piperidine-1-carboxylate

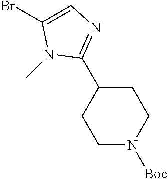 [SMILES: Cn1c(Br)cnc1C1CCN(C(=O)OC(C)(C)C)CC1]

Step 3. To a solution of tert-butyl 4-(1-methyl-1H-imidazol-2-yl) piperidine-1-carboxylate (4.8 g, 18 mmol) in ACN (60 mL), NBS (2.6 g, 14 mmol) was added. The mixture was stirred at room temperature for 1 hour. The reaction was evaporated in vacuo. The crude residue was purified by flash (Mobile Phase A: Water (10 mmol/L NH₄HCO₃), Mobile Phase B: ACN; Flow rate: 80 mL/min; Gradient: 70% B to 100% B in 7 min). Solvent evaporation afforded tert-butyl 4-(5-bromo-1-methyl-1H-imidazol-2-yl) piperidine-1-carboxylate (4.3 g, 12 mmol, 69%) as an orange oil. m/z (ES⁺) [M+H]⁺=344.15; HPLC tR=0.838 min.

### (2-(1-(tert-butoxycarbonyl) piperidin-4-yl)-1-methyl-1H-imidazol-5-yl) boronic acid

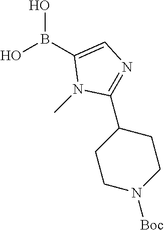 [SMILES: Cn1c(B(O)O)cnc1C1CCN(C(=O)OC(C)(C)C)CC1]

Step 4. 4,4,4′,4′,5,5,5′,5′-octamethyl-2,2′-bi(1,3,2-dioxaborolane) (6 g, 0.02 mol) and potassium acetate (5 g, 0.05 mol) were added to a solution of tert-butyl 4-(5-bromo-1-methyl-1H-imidazol-2-yl) piperidine-1-carboxylate (4 g, 0.01 mol) in Dioxane (80 mL). After bubbling nitrogen through the reaction mixture for 1 minute, dichloro(tricyclohexylphosphine)palladium (II) (4 g, 6 mmol) was added. The reaction mixture was heated at 100° C. overnight with vigorous stirring. After cooling, the crude solution was purified by flash (Mobile Phase A: Water (0.1% TFA), Mobile Phase B: ACN; Flow rate: 80 mL/min; Gradient: 23% B to 40% B in 8 min). Solvent evaporation afforded (2-(1-(tert-butoxycarbonyl) piperidin-4-yl)-1-methyl-1H-imidazol-5-yl) boronic acid (694 mg, 2.24 mmol, 20%) crude as a white solid. m/z (ES⁺) [M+H]-=310.15; HPLC tR=0.763 min.

### tert-butyl 4-(5-(2,5-dichloropyrimidin-4-yl)-1-methyl-1H-imidazol-2-yl) piperidine-1-carboxylate

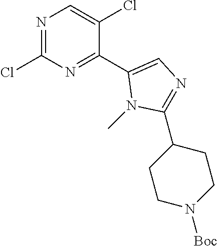 [SMILES: Cn1c(-c2nc(Cl)ncc2Cl)cnc1C1CCN(C(=O)OC(C)(C)C)CC1]

Step 5. 2,4,5-trichloropyrimidine (443 mg, 2.42 mmol) and Na₂CO₃ (698 ng, 6.59 mmol) were added to a solution of (2-(1-(tert-butoxycarbonyl) piperidin-4-yl)-1-methyl-1H-imidazol-5-yl) boronic acid (679 mg, 2.20 mmol) in dioxane (6.0 mL) and water (1.5 mL). After bubbling nitrogen through the reaction mixture for 1 minute, PdCl₂(dppf)-CH₂Cl₂ adduct (269 mg, 329 μmol) was added. The reaction mixture was heated at 80° C. for 1 hour with vigorous stirring. After cooling, the solution was purified by flash (Mobile Phase A: Water (10 mmol/L NH₄HCO₃), Mobile Phase B: ACN; Flow rate: 70 mL/min; Gradient: 42% B to 80% B in 8 min). Solvent evaporation afforded tert-butyl 4-(5-(2,5-dichloropyrimidin-4-yl)-1-methyl-1H-imidazol-2-yl) piperidine-1-carboxylate (140 mg, 340 mol, 15.5%) as a light yellow solid. m/z (ES⁺) [M+H]⁺=412.05; HPLC tR=0.981 min.

### tert-butyl 4-(5-(5-chloro-2-(((3S,4R)-3-hydroxytetrahydro-2H-pyran-4-yl) amino) pyrimidin-4-yl)-1-methyl-1H-imidazol-2-yl) piperidine-1-carboxylate

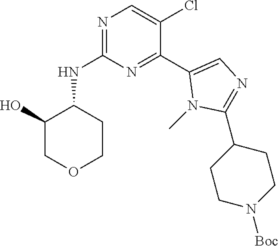 [SMILES: Cn1c(-c2nc(N[C@@H]3CCOC[C@H]3O)ncc2Cl)cnc1C1CCN(C(=O)OC(C)(C)C)CC1]

Step 6. To a solution of tert-butyl 4-(5-(2,5-dichloropyrimidin-4-yl)-1-methyl-1H-imidazol-2-yl) piperidine-1-carboxylate (135 mg, 327 μmol) in NMP (2 mL), (3S,4R)-4-aminotetrahydro-2H-pyran-3-ol hydrochloride (75.4 mg, 491 μmol) and N-ethyl-N-isopropylpropan-2-amine (169 mg, 1.31 mmol) were added. The mixture was heated under 150° C. for 0.5 hour. After cooling, the crude residue was purified by flash (Mobile Phase A: Water (10 mmol/L NH₄HCO₃), Mobile Phase B: ACN; Flow rate: 70 mL/min; Gradient: 40% B to 80% B in 8 min). Solvent evaporation afforded tert-butyl 4-(5-(5-chloro-2-(((3S,4R)-3-hydroxytetrahydro-2H-pyran-4-yl) amino) pyrimidin-4-yl)-1-methyl-1H-imidazol-2-yl) piperidine-1-carboxylate (125 mg, 254 mol, 77.4%) as a brown solid. m/z (ES⁺) [M+H]⁺=493.20; HPLC tR=0.761 min.

### (3S,4R)-4-((5-chloro-4-(1-methyl-2-(piperidin-4-yl)-1H-imidazol-5-yl)pyrimidin-2-yl) amino) tetrahydro-2H-pyran-3-ol

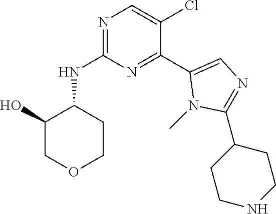 [SMILES: Cn1c(-c2nc(N[C@@H]3CCOC[C@H]3O)ncc2Cl)cnc1C1CCNCC1]

Step 7. To a solution of tert-butyl 4-(5-(5-chloro-2-(((3S,4R)-3-hydroxytetrahydro-2H-pyran-4-yl) amino) pyrimidin-4-yl)-1-methyl-1H-imidazol-2-yl) piperidine-1-carboxylate (115 mg, 233 mol) in MeOH (3 mL), HCl/dioxane (1.2 mL, 4 molar) was added. The mixture was stirred at room temperature for 0.5 hour. The reaction was adjusted PH=8 by NaHCO₃ aqueous solution. After solvent evaporation afforded (3S,4R)-4-((5-chloro-4-(1-methyl-2-(piperidin-4-yl)-1H-imidazol-5-yl)pyrimidin-2-yl) amino) tetrahydro-2H-pyran-3-ol (90 mg, 0.23 mmol, 98%) crude as an off-white solid. m/z (ES⁺) [M+H]⁺=393.20; HPLC tR=0.508 min.

### (3S,4R)-4-((5-chloro-4-(1-methyl-2-(1-methylpiperidin-4-yl)-1H-imidazol-5-yl) pyrimidin-2-yl) amino) tetrahydro-2H-pyran-3-ol

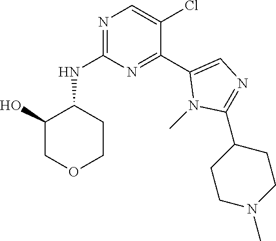 [SMILES: CN1CCC(c2ncc(-c3nc(N[C@@H]4CCOC[C@H]4O)ncc3Cl)n2C)CC1]

Step 8. To a solution of (3S,4R)-4-((5-chloro-4-(1-methyl-2-(piperidin-4-yl)-1H-imidazol-5-yl) pyrimidin-2-yl) amino)tetrahydro-2H-pyran-3-ol (90 mg, 0.23 mmol, crude) in MeOH (3 mL), Paraformaldehyde (0.28 g, 9.2 mmol), Sodium cyanoborohydride (0.14 g, 2.3 mmol) and 1 drop of AcOH were added. The mixture was stirred at room temperature for 2 h. The mixture was purified by Prep-HPLC (Column: YMC-Actus Triart C18, 19×250 mm, 5 μm; Mobile Phase A: Water (10 mmol/L NH₄HCO₃)+0.05% NH₃·H₂O, Mobile Phase B: ACN; Flow rate: 25 mL/min mL/min; Gradient: 33% B to 58% B in 7 min; Wavelength: 254 nm/220 nm nm; RT1 (min): 7.07). Lyophilization yielded (3S,4R)-4-((5-chloro-4-(1-methyl-2-(1-methylpiperidin-4-yl)-1H-imidazol-5-yl) pyrimidin-2-yl) amino) tetrahydro-2H-pyran-3-ol (26.4 mg, 64.9 mol, 28%) as an off-white solid. m/z (ES⁺) [M+H]⁺=407.15; HPLC tR=0.533 min. ¹H NMR (400 MHz, DMSO-d₆) δ 8.33 (s, 1H), 7.60 (s, 1H), 7.33 (d, J=8.3 Hz, 1H), 4.92 (d, J=5.2 Hz, 1H), 3.81 (s, 6H), 3.46 (dq, J=9.4, 4.8 Hz, 1H), 3.35 (s, 1H), 3.03 (dd, J=11.1, 9.7 Hz, 1H), 2.89-2.73 (m, 3H), 2.19 (s, 3H), 1.99 (td, J=11.3, 4.5 Hz, 2H), 1.88 (d, J=12.9 Hz, 1H), 1.79 (td, J=10.3, 9.3, 3.4 Hz, 4H), 1.48 (qd, J=12.0, 4.5 Hz, 1H).

### Example 48

### (3S,4R)-4-((5-chloro-4-(2-(1-methylpiperidin-4-yl)oxazol-5-yl)pyridin-2-yl)amino)tetrahydro-2H-pyran-3-ol (I-970)

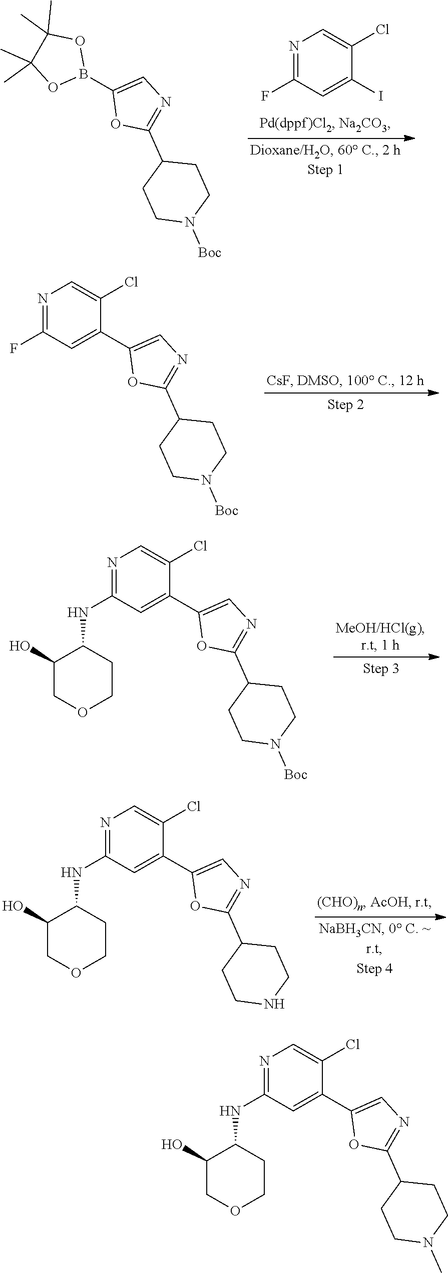

### tert-butyl 4-(5-(5-chloro-2-fluoropyridin-4-yl)oxazol-2-yl)piperidine-1-carboxylate

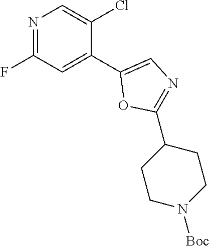 [SMILES: CC(C)(C)OC(=O)N1CCC(c2ncc(-c3cc(F)ncc3Cl)o2)CC1]

Step 1. A resealable reaction vial was charged with tert-butyl 4-(5-(4,4,5,5-tetramethyl-1,3,2-dioxaborolan-2-yl)oxazol-2-yl)piperidine-1-carboxylate (900 mg, 2.38 mmol), 5-chloro-2-fluoro-4-iodopyridine (919 mg, 3.57 mmol), Na₂CO₃ (757 mg, 7.14 mmol), Pd(dppf)Cl₂ (194 mg, 0.24 mmol), Dioxane/H₂O (1 mL) and a stirbar before being evacuated and purged with nitrogen three times, and the mixture was stirred for 2 h at 60° C. The solution was purified by reverse flash chromatography with the following conditions: column, C18 silica gel; mobile phase, MeCN in water, 10% to 50% gradient in 20 min; detector, UV 254 nm. Concentration in vacuo resulted in tert-butyl 4-(5-(5-chloro-2-fluoropyridin-4-yl)oxazol-2-yl)piperidine-1-carboxylate (66 mg, 73%) as a colorless oil. m/z (ES⁺) [M+H]⁺=382.25; HPLC tR=0.888 min.

### tert-butyl 4-(5-(5-chloro-2-(((3S,4R)-3-hydroxytetrahydro-2H-pyran-4-yl)amino)pyridin-4-yl)oxazol-2-yl)piperidine-1-carboxylate

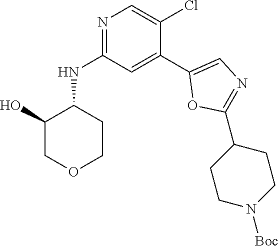 [SMILES: CC(C)(C)OC(=O)N1CCC(c2ncc(-c3cc(N[C@@H]4CCOC[C@H]4O)ncc3Cl)o2)CC1]

Step 2. A resealable reaction vial was charged with tert-butyl 4-(5-(5-chloro-2-fluoropyridin-4-yl)oxazol-2-yl)piperidine-1-carboxylate (41 mg, 0.11 mmol), (3S,4R)-4-aminotetrahydro-2H-pyran-3-ol hydrochloride (18 mg, 0.12 mmol), CsF (82 mg, 0.54 mmol), DMSO (1 mL) and a stirbar before being evacuated and purged with nitrogen three times, and the mixture was stirred for 12 h at 100° C. The mixture was purified by reverse flash chromatography with the following conditions: column, C18 silica gel; mobile phase, MeCN in water, 10% to 50% gradient in 20 min; detector, UV 254 nm. Concentration in vacuo resulted in tert-butyl 4-(5-(5-chloro-2-(((3S,4R)-3-hydroxytetrahydro-2H-pyran-4-yl)amino)pyridin-4-yl)oxazol-2-yl)piperidine-1-carboxylate (16 mg, 31%) as a white solid. m/z (ES⁺) [M+H]⁺=479.25; HPLC tR=0.630 min.

### (3S,4R)-4-((5-chloro-4-(2-(piperidin-4-yl)oxazol-5-yl)pyridin-2-yl)amino)tetrahydro-2H-pyran-3-ol

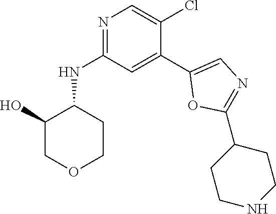 [SMILES: O[C@@H]1COCC[C@H]1Nc1cc(-c2cnc(C3CCNCC3)o2)c(Cl)cn1]

Step 3. A resealable reaction vial was charged with tert-butyl 4-(5-(5-chloro-2-(((3S,4R)-3-hydroxytetrahydro-2H-pyran-4-yl)amino)pyridin-4-yl)oxazol-2-yl)piperidine-1-carboxylate (16 mg, 33 mol), MeOH/HCl(g) (1 mL, 4 molar) and a stirbar before being evacuated and purged with nitrogen three times, and the mixture was stirred for 1 hour at 25° C. and concentrated in vacuo, then adjusted to pH 7-8 with sodium bicarbonate solution. Concentration in vacuo resulted in crude (3S,4R)-4-((5-chloro-4-(2-(piperidin-4-yl)oxazol-5-yl)pyridin-2-yl)amino)tetrahydro-2H-pyran-3-ol (13 mg, 100%) as a white solid. m/z (ES⁺) [M+H]⁺=379.00; HPLC tR=0.117 min.

### (3S,4R)-4-((5-chloro-4-(2-(1-methylpiperidin-4-yl)oxazol-5-yl)pyridin-2-yl)amino)tetrahydro-2H-pyran-3-ol

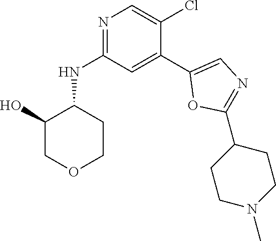 [SMILES: CN1CCC(c2ncc(-c3cc(N[C@@H]4CCOC[C@H]4O)ncc3Cl)o2)CC1]

Step 4. To a mixture of (3S,4R)-4-((4-(2-(piperidin-4-yl)oxazol-5-yl)pyridin-2-yl)amino)tetrahydro-2H-pyran-3-ol (13 mg, 0.04 mmol) and AcOH (0.2 mg, 0.004 mmol) in MeOH (1 mL) was added Paraformaldehyde (3 mg, 0.11 mmol) in portions at 25° C. under nitrogen atmosphere. The mixture was stirred for 30 min at 0° C. prior addition of NaBH₃CN (7.1 mg, 0.11 mmol). The mixture was stirred for 1 h at 25° C. The solution was purified by reverse flash chromatography with the following conditions: column, C18 silica gel; mobile phase, MeCN in water, 10% to 50% gradient in 20 min; detector, UV 254 nm, Concentration in vacuo. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 13% B to 40% B in 7 min; Wavelength: 254 nm/220 nm nm; RT1 (min): 7.4). Lyophilization yielded (3S,4R)-4-((5-chloro-4-(2-(1-methylpiperidin-4-yl)oxazol-5-yl)pyridin-2-yl)amino)tetrahydro-2H-pyran-3-ol (2 mg, 10%) as a white solid. m/z (ES⁺) [M+H]⁺=393.05; HPLC tR=1.318 min. ¹H NMR (400 MHz, DMSO-d₆) δ 8.06 (s, 1H), 7.80 (s, 1H), 6.95 (d, J=8.3 Hz, 2H), 5.01 (d, J=5.2 Hz, 1H), 3.79 (td, J=11.2, 4.5 Hz, 3H), 3.38 (d, J=4.2 Hz, 2H), 3.07 (dd, J=11.1, 9.3 Hz, 1H), 2.93-2.84 (m, 1H), 2.77 (d, J=11.2 Hz, 2H), 2.18 (s, 3H), 2.05-1.97 (m, 5H), 1.84-1.72 (m, 2H), 1.43-1.31 (m, 1H).

### Example 49

### (3S,4R)-4-((5-chloro-4-(4-methyl-3-(1-methylpiperidin-4-yl)isothiazol-5-yl)pyridin-2-yl)amino)tetrahydro-2H-pyran-3-ol (I-1043)

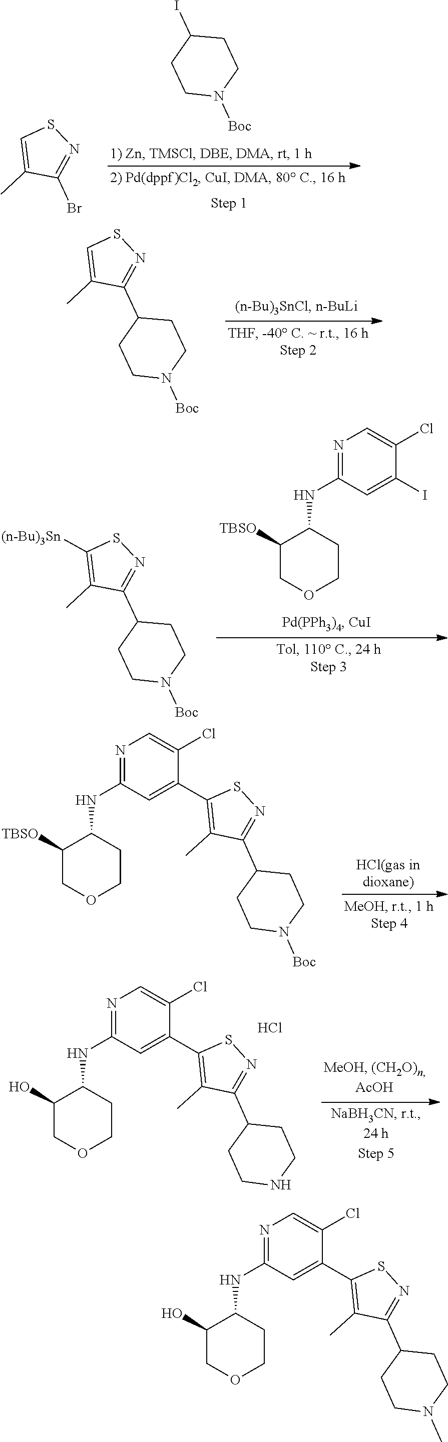

### tert-butyl 4-(4-methylisothiazol-3-yl)piperidine-1-carboxylate

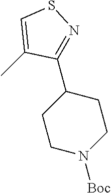 [SMILES: Cc1csnc1C1CCN(C(=O)OC(C)(C)C)CC1]

Step 1. A round bottomed flask was charged with zinc (0.8 g, 2.2 Eq, 0.01 mol), DMA (14 mL) and a stirbar before being evacuated and purged with nitrogen three times. 1,2-dibromoethane (0.1 g, 0.1 Eq, 0.6 mmol) and TMS-C₁ (0.06 g, 0.07 mL, 0.1 Eq, 0.6 mmol) were added. The mixture was stirred at 20° C. for 30 min. Tert-butyl 4-iodopiperidine-1-carboxylate (3 g, 1.5 Eq, 8 mmol) in DMA (14 mL) was added dropwise into the mixture. The mixture was stirred at 20° C. for 2 hours to result in Zn-reagent. A resealable reaction vial was charged with 3-bromo-4-methylisothiazole (1 g, 1 Eq, 6 mmol), Pd (dppf) Cl₂ (0.4 g, 0.1 Eq, 0.6 mmol), CuI (0.1 g, 0.1 Eq, 0.6 mmol), DMA (14 mL) and a stirbar before being evacuated and purged with nitrogen three times. The Zn-reagent was filtered and the filtrate was added into the above solution. The mixture was stirred at 80° C. for 16 hours. The mixture was diluted with water (250 mL), and the aqueous phase was extracted with EA (100 mL) three times. The combined organic layers were washed with brine, dried over sodium sulfate, filtered, concentrated in vacuo, and purified by Flash (acetonitrile/water). Concentration in vacuo. The resulting crude material was purified by prep-TLC (PE/EA=4:1). Concentration in vacuo resulted in tert-butyl 4-(4-methylisothiazol-3-yl)piperidine-1-carboxylate (200 mg, 708 mol, 10%) as a colorless oil. m/z (ES⁺) [M+H]⁺=283.00; HPLC tR=1.021 min.

### tert-butyl 4-(4-methyl-5-(tributylstannyl)isothiazol-3-yl)piperidine-1-carboxylate

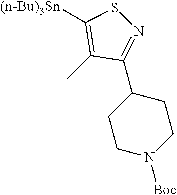

Step 2. A resealable reaction vial was charged with tert-butyl 4-(4-methylisothiazol-3-yl)piperidine-1-carboxylate (200 mg, 1 Eq, 708 mol), THE (5 mL) and a stirbar before being evacuated and purged with nitrogen three times. N-butyllithium (2.5M in hexane, 0.85 mL, 3 Eq, 2.12 mmol) was added dropwise at −40° C. The mixture was stirred at −40° C. for 30 min. Tributylchlorostannane (1.38 g, 6 Eq, 4.25 mmol) was added dropwise at −40° C. The mixture was stirred at 20° C. for 16 hours. The reaction mixture was quenched with sat. NH₄Cl and purified by flash chromatography (acetonitrile/water). Concentration in vacuo yielded tert-butyl 4-(4-methyl-5-(tributylstannyl)isothiazol-3-yl)piperidine-1-carboxylate (280 mg, 490 μmol, 69.2%) as a colorless oil. m/z (ES⁺) [M+H]⁺=573.30; HPLC tR=1.243 min.

### tert-butyl 4-(5-(2-(((3S,4R)-3-((tert-butyldimethylsilyl)oxy)tetrahydro-2H-pyran-4-yl)amino)-5-chloropyridin-4-yl)-4-methylisothiazol-3-yl)piperidine-1-carboxylate

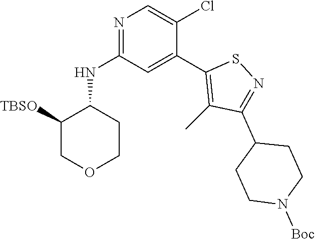 [SMILES: Cc1c(C2CCN(C(=O)OC(C)(C)C)CC2)nsc1-c1cc(N[C@@H]2CCOC[C@H]2O[Si](C)(C)C(C)(C)C)ncc1Cl]

Step 3. A resealable reaction vial was charged with tert-butyl 4-(4-methyl-5-(tributylstannyl)isothiazol-3-yl)piperidine-1-carboxylate (220 mg, 1 eq, 385 mol), N-((3S,4R)-3-((tert-butyldimethylsilyl)oxy) tetrahydro-2H-pyran-4-yl)-5-chloro-4-iodopyridin-2-amine (199 mg, 1.1 Eq, 423 μmol), CuI (147 mg, 2 Eq, 770 μmol), Pd(PPh₃)₄ (44.5 mg, 0.1 Eq, 38.5 mol), Toluene (10 mL) and a stirbar before being evacuated and purged with nitrogen three times. The resulting mixture was stirred for 24 hours at 110° C. The crude product was concentrated under vacuum and purified by reverse flash chromatography with the following conditions: column, C18 silica gel; mobile phase, MeCN in water, 75% to 100% gradient in 20 min; detector, UV 254 nm and concentrated in vacuo to afford tert-butyl 4-(5-(2-(((3S,4R)-3-((tert-butyldimethylsilyl)oxy)tetrahydro-2H-pyran-4-yl)amino)-5-chloropyridin-4-yl)-4-methylisothiazol-3-yl)piperidine-1-carboxylate (300 mg, 0.34 mmol, 88%, 70% purity) as yellow oil. m/z (ES⁺) [M+H]⁺=623.45; HPLC tR=1.212 min.

### (3S,4R)-4-((5-chloro-4-(4-methyl-3-(piperidin-4-yl)isothiazol-5-yl)pyridin-2-yl)amino)tetrahydro-2H-pyran-3-ol hydrochloride

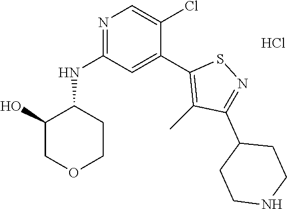 [SMILES: Cc1c(C2CCNCC2)nsc1-c1cc(N[C@@H]2CCOC[C@H]2O)ncc1Cl ; Cl]

Step 4. To a solution of tert-butyl 4-(5-(2-(((3S,4R)-3-((tert-butyldimethylsilyl)oxy)tetrahydro-2H-pyran-4-yl)amino)-5-chloropyridin-4-yl)-4-methylisothiazol-3-yl)piperidine-1-carboxylate (295 mg, 1 Eq, 473 μmol) in MeOH (1.5 mL) was added HCl (4M in 1,4-Dioxane) (1.5 mL). The solution was stirred for 1 hour at 25° C. The resulting mixture was concentrated under vacuum to afford (3S,4R)-4-((5-chloro-4-(4-methyl-3-(piperidin-4-yl)isothiazol-5-yl)pyridin-2-yl)amino)tetrahydro-2H-pyran-3-ol hydrochloride (240 mg, 0.38 mmol, 80%, 70% Purity) as yellow oil. m/z (ES⁺) [M+H]⁺=409.05; HPLC tR=0.540 min.

### (3S,4R)-4-((5-chloro-4-(4-methyl-3-(1-methylpiperidin-4-yl)isothiazol-5-yl)pyridin-2-yl)amino)tetrahydro-2H-pyran-3-ol

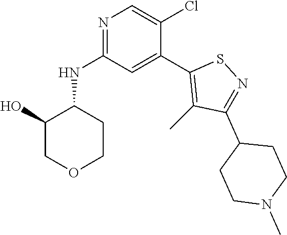 [SMILES: Cc1c(C2CCN(C)CC2)nsc1-c1cc(N[C@@H]2CCOC[C@H]2O)ncc1Cl]

Step 5. To a solution of (3S,4R)-4-((5-chloro-4-(4-methyl-3-(piperidin-4-yl)isothiazol-5-yl)pyridin-2-yl)amino)tetrahydro-2H-pyran-3-ol hydrochloride (120 mg, 1 Eq, 269 mol) in MeOH (8 mL) was added formaldehyde (162 mg, 20 Eq, 5.39 mmol) and AcOH (1.62 mg, 1.54 μL, 0.1 Eq, 26.9 mol) at 25° C. Then sodium cyanotrihydroborate (169 mg, 10 Eq, 2.69 mmol) was added to the above solution at 0° C. in portions. The mixture was stirred for 24 hours at 25° C. The resulting crude material was purified by Prep-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 18% B to 45% B in 7 min; Wavelength: 254 nm/220 nm nm; RT1 (min): 7.67). Lyophilization yielded (3S,4R)-4-((5-chloro-4-(4-methyl-3-(1-methylpiperidin-4-yl)isothiazol-5-yl)pyridin-2-yl)amino)tetrahydro-2H-pyran-3-ol (10.6 mg, 24.4 μmol, 9.06%, 97.388% purity) as a white solid. m/z (ES⁺) [M+H]⁺=423.25; HPLC tR=1.358 min. ¹H NMR (400 MHz, DMSO-d₆) 8.12 (s, 1H), 6.93 (d, J=7.5 Hz, 1H), 6.55 (s, 1H), 5.00 (d, J=5.4 Hz, 1H), 3.85-3.67 (m, 3H), 3.39 (dd, J=9.3, 4.1 Hz, 2H), 3.06 (dd, J=11.1, 9.5 Hz, 1H), 2.88 (d, J=10.7 Hz, 2H), 2.79 (s, 1H), 2.21 (s, 3H), 2.09 (s, 3H), 1.99 (d, J=14.0 Hz, 3H), 1.78 (d, J=14.6 Hz, 4H), 1.51-1.31 (m, 1H).

### Example 50

### (3S,4R)-4-((5-chloro-4-(3-(1-methylpiperidin-4-yl)-1,2,4-thiadiazol-5-yl)pyridin-2-yl)amino)tetrahydro-2H-pyran-3-ol (I-978)

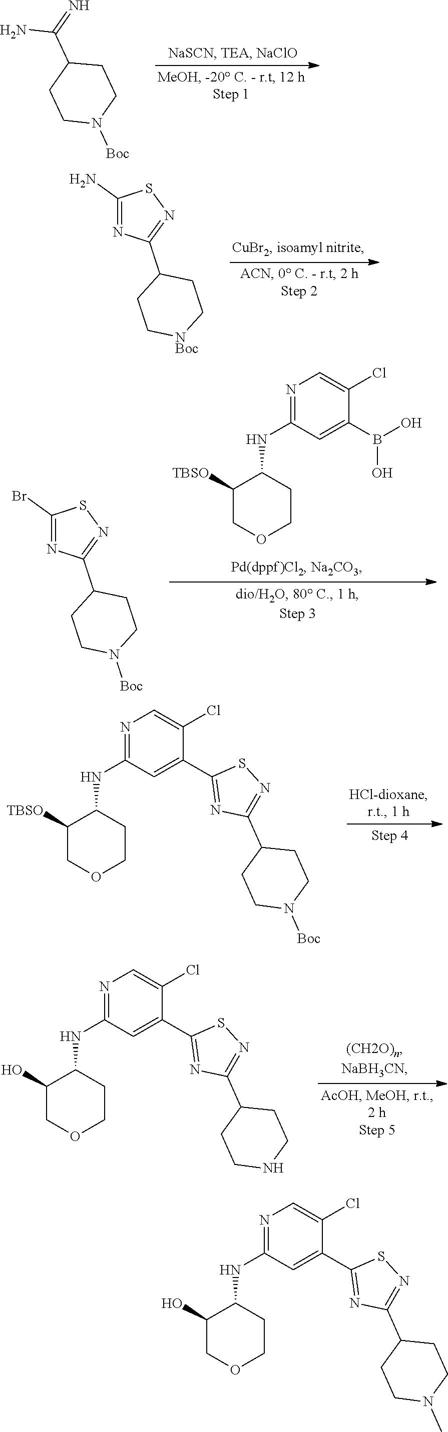

### tert-butyl 4-(5-amino-1,2,4-thiadiazol-3-yl)piperidine-1-carboxylate

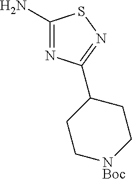 [SMILES: CC(C)(C)OC(=O)N1CCC(c2nsc(N)n2)CC1]

Step 1. A mixture of sodium thiocyanate (0.70 g, 1.3 Eq, 8.6 mmol) in MeOH (20 mL) was stirred for 5 min at −20° C. Triethylamine (1 g, 1.4 Eq, 10 mmol) and tert-butyl 4-carbamimidoylpiperidine-1-carboxylate (1.5 g, 1 Eq, 6.6 mmol) were added in portions at −20° C. under nitrogen atmosphere. The mixture was stirred for 45 min at −20° C. Then to the above mixture triethylamine (0.5 g, 0.8 Eq, 5 mmol) and sodium hypochlorite (0.49 g, 1 Eq, 6.6 mmol) in water (5%) were added, and the resulting reaction was stirred at −20° C. for 2 hours and another 10 hours at 28° C. The mixture was concentrated under reduced pressure. The residue was diluted with water and extracted with EtOAc (3×30 ml). The combined organic layers were washed with brine (1×20 mL), dried over anhydrous Na₂SO₄. After filtration, the filtrate was concentrated under reduced pressure to afford crude tert-butyl 4-(5-amino-1,2,4-thiadiazol-3-yl)piperidine-1-carboxylate (800 mg, 2.81 mmol, 43%) as a white solid. m/z (ES⁺) [M+H-t-Bu]⁺=229.00; HPLC tR=0.738 min.

### tert-butyl 4-(5-bromo-1,2,4-thiadiazol-3-yl)piperidine-1-carboxylate

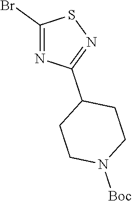 [SMILES: CC(C)(C)OC(=O)N1CCC(c2nsc(Br)n2)CC1]

Step 2. To a mixture of tert-butyl 4-(5-amino-1,2,4-thiadiazol-3-yl)piperidine-1-carboxylate (0.73 g, 1 Eq, 2.6 mmol) and copper(II) bromide (0.86 g, 1.5 Eq, 3.9 mmol) in MeCN (10 mL), isopentyl nitrite (0.45 g, 1.5 Eq, 3.9 mmol) was added dropwise at 0° C. under nitrogen atmosphere. The mixture was stirred for 2 h at 28° C. The mixture was concentrated under reduced pressure. The residue was purified by silica gel column chromatography (eluted with PE:EA=8:1) to afford tert-butyl 4-(5-bromo-1,2,4-thiadiazol-3-yl)piperidine-1-carboxylate (0.38 g, 1.1 mmol, 43%) as a white solid. m/z (ES⁺) [M+H-t-Bu]⁺=291.90; HPLC tR=1.077 min.

### tert-butyl 4-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperidine-1-carboxylate

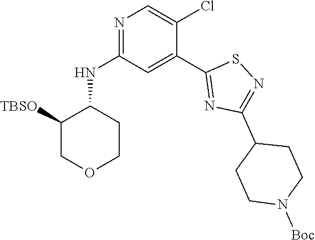 [SMILES: CC(C)(C)OC(=O)N1CCC(c2nsc(-c3cc(N[C@@H]4CCOC[C@H]4O[Si](C)(C)C(C)(C)C)ncc3Cl)n2)CC1]

Step 3. To a solution of (2-(((3S,4R)-3-((tert-butyldimethylsilyl)oxy)tetrahydro-2H-pyran-4-yl)amino)-5-chloropyridin-4-yl)boronic acid (999 mg, 2.5 Eq, 2.58 mmol) and tert-butyl 4-(5-bromo-1,2,4-thiadiazol-3-yl)piperidine-1-carboxylate (360 mg, 1 Eq, 1.03 mmol) in 1,4-dioxane (4 mL) and Water (1 mL), sodium carbonate (329 mg, 3 Eq, 3.10 mmol) and Pd(dppf)Cl₂ CH₂Cl₂ (84.4 mg, 0.1 Eq, 103 μmol) were added. After stirring for 1 hour at 80° C. under a nitrogen atmosphere. The reaction was diluted with water (10 ml), and then the mixture was extracted with EtOAc (2×50 ml). The combined organic extracts were washed with brine 40 ml, dried over anhydrous Na₂SO₄, and concentrated under vacuum. The residue was purified by silica gel column chromatography (eluted with PE:EA=10:1) to afford tert-butyl 4-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperidine-1-carboxylate (380 mg, 623 mol, 60.2%) as a white solid. m/z (ES⁺) [M+H]⁺=610.20; HPLC tR=1.217 min.

### (3S,4R)-4-((5-chloro-4-(3-(piperidin-4-yl)-1,2,4-thiadiazol-5-yl)pyridin-2-yl)amino)tetrahydro-2H-pyran-3-ol

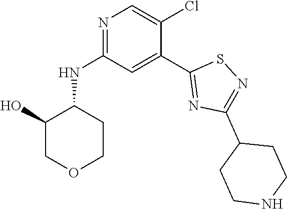 [SMILES: O[C@@H]1COCC[C@H]1Nc1cc(-c2nc(C3CCNCC3)ns2)c(Cl)cn1]

Step 4. A round bottom flask was charged with tert-butyl 4-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperidine-1-carboxylate (370 mg, 1 Eq, 606 mol) in HCl-dioxane (20 mL, 4 molar), the solution was stirred for 5 hours at 28° C. The mixture was concentrated under reduced pressure to afford product (3S,4R)-4-((5-chloro-4-(3-(piperidin-4-yl)-1,2,4-thiadiazol-5-yl)pyridin-2-yl)amino)tetrahydro-2H-pyran-3-ol (260 mg, 0.53 mmol, 87%, 80% Purity) as a white solid. m/z (ES⁺) [M+H]⁺=396.15; HPLC tR=0.855 min.

### (3S,4R)-4-((5-chloro-4-(3-(1-methylpiperidin-4-yl)-1,2,4-thiadiazol-5-yl)pyridin-2-yl)amino)tetrahydro-2H-pyran-3-ol

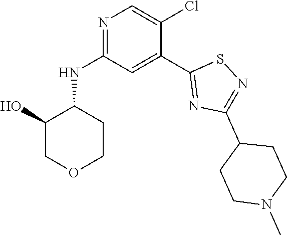 [SMILES: CN1CCC(c2nsc(-c3cc(N[C@@H]4CCOC[C@H]4O)ncc3Cl)n2)CC1]

Step 5. A round bottom flask was charged with (3S,4R)-4-((5-chloro-4-(3-(piperidin-4-yl)-1,2,4-thiadiazol-5-yl)pyridin-2-yl)amino)tetrahydro-2H-pyran-3-ol (260 mg, 1 Eq, 657 mol) and Paraformaldehyde (394 mg, 396 μL, 20 Eq, 13.1 mmol) in MeOH (10 mL), the solution was stirred for 5 min at 0° C. Then the Sodium cyanoborohydride (413 mg, 382 μL, 10 Eq, 6.57 mmol) was added in portion. The mixture was warmed up to rt and stirred for 5 h at 28° C. The reaction was quenched with H₂O, the resulting mixture was filtered, the filter cake was washed with MeOH (30 ml), the filtrate was concentrated under reduced pressure. The residue was purified by C18 chromatography (120 g, eluted with MeCN in water; ratio: 0-100%) to afford crude product (200 mg). The crude product (200 mg) was purified by Prep-HPLC with the following conditions: (Column: XBridge Prep OBD C18 Column, 30×150 mm, 5 μm; Mobile Phase A: Water (10 mmol/L NH₄HCO₄+0.05% NH₃H₂O), Mobile Phase B: ACN; Flow rate: 60 mL/min mL/min; Gradient: 21% B to 46% B in 8 min; Wavelength: 220 nm nm; RT1 (min): 7.42) to afford (3S,4R)-4-((5-chloro-4-(3-(1-methylpiperidin-4-yl)-1,2,4-thiadiazol-5-yl)pyridin-2-yl)amino)tetrahydro-2H-pyran-3-ol (85.1 mg, 207 μmol, 31.5%, 99.6% Purity) as a yellow solid. m/z (ES⁺) [M+H]⁺=410.10; HPLC tR=1.325 min. ¹H NMR (400 MHz, DMSO-d₆) 8.25 (s, 1H), 7.54 (s, 1H), 7.25 (d, J=7.6 Hz, 1H), 5.01 (d, J=5.2 Hz, 1H), 3.80 (td, J=10.8, 4.2 Hz, 3H), 3.44-3.37 (m, 2H), 3.09 (dd, J=11.1, 9.2 Hz, 1H), 2.98 (ddt, J=11.6, 7.8, 3.8 Hz, 1H), 2.89-2.79 (m, 2H), 2.21 (s, 3H), 2.10-1.96 (m, 5H), 1.93-1.78 (m, 2H), 1.38 (qd, J=11.0, 4.4 Hz, 1H).

### Example 51

### (R)-((1R,3R,4R)-4-((5-chloro-4-(2-(1-methylpiperidin-4-yl) oxazol-5-yl)pyrimidin-2-yl) amino)-3-hydroxycyclohexyl) (imino)(methyl)-λ⁶-sulfanone (I-1035), &

### (S)-((1R,3R,4R)-4-((5-chloro-4-(2-(1-methylpiperidin-4-yl) oxazol-5-yl)pyrimidin-2-yl) amino)-3-hydroxycyclohexyl) (imino)(methyl)-λ⁶-sulfanone (I-1036)

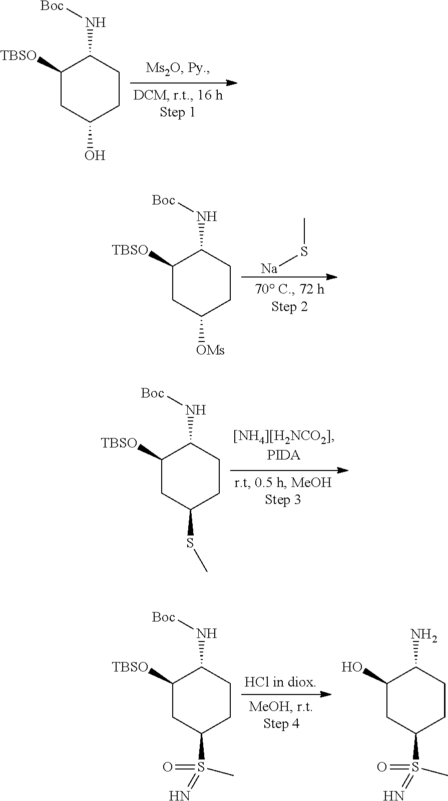

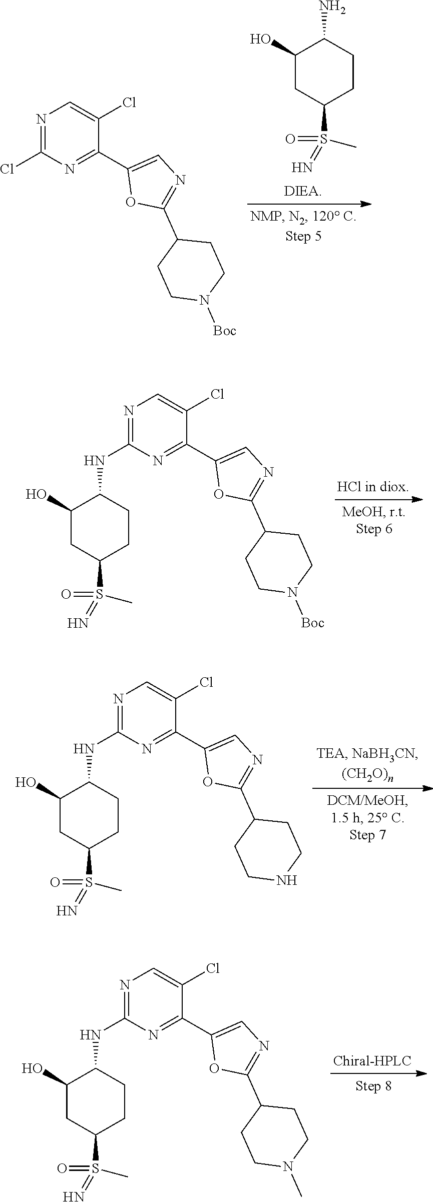 [SMILES: CC(C)(C)OC(=O)N1CCC(c2ncc(-c3nc(Cl)ncc3Cl)o2)CC1 ; CS(=N)(=O)[C@@H]1CC[C@@H](N)[C@H](O)C1 ; CC(C)(C)OC(=O)N1CCC(c2ncc(-c3nc(N[C@@H]4CC[C@@H](S(C)(=N)=O)C[C@H]4O)ncc3Cl)o2)CC1 ; CS(=N)(=O)[C@@H]1CC[C@@H](Nc2ncc(Cl)c(-c3cnc(C4CCNCC4)o3)n2)[C@H](O)C1 ; CN1CCC(c2ncc(-c3nc(N[C@@H]4CC[C@@H](S(C)(=N)=O)C[C@H]4O)ncc3Cl)o2)CC1]

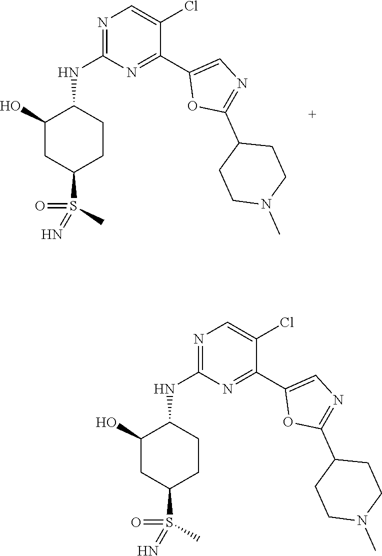 [SMILES: CN1CCC(c2ncc(-c3nc(N[C@@H]4CC[C@@H]([S@](C)(=N)=O)C[C@H]4O)ncc3Cl)o2)CC1 ; CN1CCC(c2ncc(-c3nc(N[C@@H]4CC[C@@H]([S@@](C)(=N)=O)C[C@H]4O)ncc3Cl)o2)CC1]

### (1S,3R,4R)-4-((tert-butoxycarbonyl) amino)-3-((tert-butyldimethylsilyl) oxy) cyclohexyl methanesulfonate

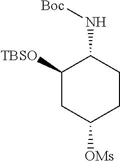 [SMILES: CC(C)(C)OC(=O)N[C@@H]1CC[C@H](OS(C)(=O)=O)C[C@H]1O[Si](C)(C)C(C)(C)C]

Step 1. To a mixture of tert-butyl ((1R,2R,4S)-2-((tert-butyldimethylsilyl) oxy)-4-hydroxycyclohexyl) carbamate (1.8 g, 5.2 mmol) in DCM (12 mL) was added pyridine (2.1 g, 2.1 mL, 26 mmol), then methanesulfonic anhydride (2.7 g, 6 mmol) was added at 0° C. under nitrogen atmosphere. The mixture was stirred at 25° C. for 16 hours. The reaction mixture was concentrated in vacuo. The residue was purified by reverse flash chromatography with the following conditions: column, C18 silica gel; mobile phase, ACN in water, 60% to 87% gradient in 15 min; detector, UV 200 nm. The combined organic layers were concentrated in vacuo and get (1S,3R,4R)-4-((tert-butoxycarbonyl) amino)-3-((tert-butyldimethylsilyl) oxy) cyclohexyl methanesulfonate (1.9 g, 4.5 mmol, 86%) as a clear oil. m/z (ES⁺) [M+H]⁺=424.25; HPLC tR=1.005 min. m/z (ES⁺) [M+H]⁺=424.25; HPLC tR=1.005 min.

### tert-butyl ((1R,2R,4R)-2-((tert-butyldimethylsilyl) oxy)-4-(methylthio) cyclohexyl) carbamate

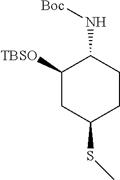 [SMILES: CS[C@@H]1CC[C@@H](NC(=O)OC(C)(C)C)[C@H](O[Si](C)(C)C(C)(C)C)C1]

Step 2. A mixture of sodium methanethiolate (3.1 g, 45 mmol) in MeOH (4.5 mL) was stirred at r.t. for 10 min. (1S,3R,4R)-4-((tert-butoxycarbonyl)amino)-3-((tert-butyldimethylsilyl)oxy)cyclohexyl methanesulfonate (1.9 g, 4.5 mmol) in EtOH (9 mL) was added at room temperature. The mixture was stirred at 70° C. for 72 hours. The reaction mixture was concentrated in vacuo. The residue was purified by reverse flash chromatography with the following conditions: column, C18 silica gel; mobile phase, ACN in water, 60% to 100% gradient in 20 min; detector, UV 200 nm. The combined organic layers were concentrated in vacuo to get tert-butyl ((1R,2R,4R)-2-((tert-butyldimethylsilyl) oxy)-4-(methylthio) cyclohexyl) carbamate (1.11 g, 2.95 mmol, 66%) as a yellow oil. m/z (ES⁺) [M+H]⁺=376.30; HPLC tR=1.130 min.

### tert-butyl ((1R,2R,4R)-2-((tert-butyldimethylsilyl) oxy)-4-(S-methylsulfonimidoyl) cyclohexyl) carbamate

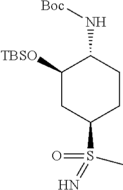 [SMILES: CC(C)(C)OC(=O)N[C@@H]1CC[C@@H](S(C)(=N)=O)C[C@H]1O[Si](C)(C)C(C)(C)C]

Step 3. To a mixture of tert-butyl ((1R,2R,4R)-2-((tert-butyldimethylsilyl)oxy)-4-(methylthio)cyclohexyl)carbamate (1.1 g, 2.9 mmol) in MeOH (10 mL) were added ammonium carbamate (0.69 g, 8.8 mmol) and iodosobenzene diacetate (2.8 g, 8.8 mmol). The mixture was stirred at 25° C. for 0.5 hour under nitrogen atmosphere. The reaction mixture was concentrated in vacuo. The residue was purified by reverse flash chromatography with the following conditions: column, C18 silica gel; mobile phase, ACN in water, 30% to 80% gradient in 20 min; detector, UV 200 nm. The combined organic layers were concentrated in vacuo to get tert-butyl ((1R,2R,4R)-2-((tert-butyldimethylsilyl) oxy)-4-(S-methylsulfonimidoyl) cyclohexyl) carbamate (460 mg, 1.13 mmol, 39%) as a yellow solid. m/z (ES⁺) [M+H]⁺=407.30; HPLC tR=0.813 min.

### ((1R,3R,4R)-4-amino-3-hydroxycyclohexyl) (imino)(methyl)-16-sulfanone

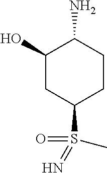

Step 4. A solution of tert-butyl ((1R,2R,4R)-2-((tert-butyldimethylsilyl)oxy)-4-(S-methylsulfonimidoyl) cyclohexyl)carbamate (110 mg, 270 μmol) in MeOH (0.6 mL) and hydrogen chloride in 1,4-dioxane solution (0.3 mL, 4M) was stirred at 25° C. for 3 hours. The solution was concentrated under vacuum to give the ((1R,3R,4R)-4-amino-3-hydroxycyclohexyl) (imino)(methyl)-X⁶-sulfanone (49 mg, 0.25 mmol, 94%) crude as a yellow solid. m/z (ES⁺) [M+H]=193.00; HPLC tR=0.138 min.

### tert-butyl 4-(5-(5-chloro-2-(((1R,2R,4R)-2-hydroxy-4-(S-methylsulfonimidoyl) cyclohexyl) amino) pyrimidin-4-yl) oxazol-2-yl)piperidine-1-carboxylate

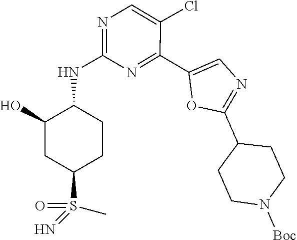 [SMILES: CC(C)(C)OC(=O)N1CCC(c2ncc(-c3nc(N[C@@H]4CC[C@@H](S(C)(=N)=O)C[C@H]4O)ncc3Cl)o2)CC1]

Step 5. To a solution of ((1R,3R,4R)-4-amino-3-hydroxycyclohexyl)(imino)(methyl)-λ⁶-sulfanone (52 mg, 0.27 mmol) in NMP (1 mL) were added N-ethyl-N-isopropylpropan-2-amine (0.35 g, 2.7 mmol), tert-butyl 4-(5-(2,5-dichloropyrimidin-4-yl) oxazol-2-yl) piperidine-1-carboxylate (0.11 g, 0.27 mmol). The mixture was stirred at 120° C. for 1 hour. The solution was purified by reverse flash chromatography with the following conditions: column, C18 silica gel; mobile phase, ACN in water, 0% to 100% gradient in 20 min; detector, UV 254 nm. Lyophilization yielded tert-butyl 4-(5-(5-chloro-2-(((1R,2R,4R)-2-hydroxy-4-(S-methylsulfonimidoyl) cyclohexyl) amino) pyrimidin-4-yl) oxazol-2-yl)piperidine-1-carboxylate (105 mg, 0.19 mmol, 69%, 98% Purity) as a light brown solid. m/z (ES⁺) [M+H]⁺=555.25; HPLC tR=0.756 min.

### ((1R,3R,4R)-4-((5-chloro-4-(2-(piperidin-4-yl) oxazol-5-yl)pyrimidin-2-yl)amino)-3-hydroxycyclohexyl)(imino)(methyl)-λ⁶-sulfanone

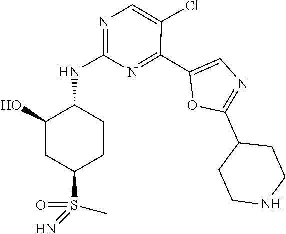 [SMILES: CS(=N)(=O)[C@@H]1CC[C@@H](Nc2ncc(Cl)c(-c3cnc(C4CCNCC4)o3)n2)[C@H](O)C1]

Step 6. A solution of tert-butyl 4-(5-(5-chloro-2-(((1R,2R,4R)-2-hydroxy-4-(S-methylsulfonimidoyl) cyclohexyl)amino)pyrimidin-4-yl)oxazol-2-yl)piperidine-1-carboxylate (105 mg, 189 μmol) in MeOH (0.5 mL) and hydrogen chloride in 1,4-dioxane solution (0.5 mL, 4M) was stirred at 25° C. for 1 hour. The solution was concentrated under vacuum to give the ((1R,3R,4R)-4-((5-chloro-4-(2-(piperidin-4-yl) oxazol-5-yl)pyrimidin-2-yl)amino)-3-hydroxycyclohexyl)(imino)(methyl)-λ⁶-sulfanone (80 mg, 0.18 mmol, 93%) crude as a yellow solid. m/z (ES⁺) [M+H]⁺=455.10, HPLC tR=0.538 min.

### ((1R,3R,4R)-4-((5-chloro-4-(2-(1-methylpiperidin-4-yl) oxazol-5-yl) pyrimidin-2-yl) amino)-3-hydroxycyclohexyl) (imino)(methyl)-46-sulfanone

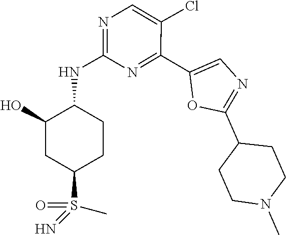 [SMILES: CN1CCC(c2ncc(-c3nc(N[C@@H]4CC[C@@H](S(C)(=N)=O)C[C@H]4O)ncc3Cl)o2)CC1]

Step 7. To a solution of ((1R,3R,4R)-4-((5-chloro-4-(2-(piperidin-4-yl) oxazol-5-yl) pyrimidin-2-yl) amino)-3-hydroxycyclohexyl)(imino)(methyl)-λ⁶-sulfanone (81 mg, 0.18 mmol) in DCM (1.5 mL) and MeOH (0.30 mL), triethylamine (72 mg, 0.71 mmol) was added. Then the solution was cooled to 0° C., sodium cyanoborhydride (34 mg, 0.53 mmol) was added. The mixture was stirred at 25° C. for 1.5 hours. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 10% B to 31% B in 7 min; Wavelength: 254 nm/220 nm nm; RT1 (min): 7.03). Lyophilization yielded ((1R,3R,4R)-4-((5-chloro-4-(2-(1-methylpiperidin-4-yl) oxazol-5-yl) pyrimidin-2-yl) amino)-3-hydroxycyclohexyl) (imino)(methyl)-λ⁶-sulfanone (40 mg, 85 μmol, 43%) as an off-white solid. m/z (ES⁺) [M+H]⁺=469.20; HPLC tR=0.482 min.

### (R)-((1R,3R,4R)-4-((5-chloro-4-(2-(1-methylpiperidin-4-yl) oxazol-5-yl) pyrimidin-2-yl) amino)-3-hydroxycyclohexyl) (imino)(methyl)-λ⁶-sulfanone &

### (S)-((1R,3R,4R)-4-((5-chloro-4-(2-(1-methylpiperidin-4-yl) oxazol-5-yl) pyrimidin-2-yl) amino)-3-hydroxycyclohexyl) (imino)(methyl)-λ⁶-sulfanone

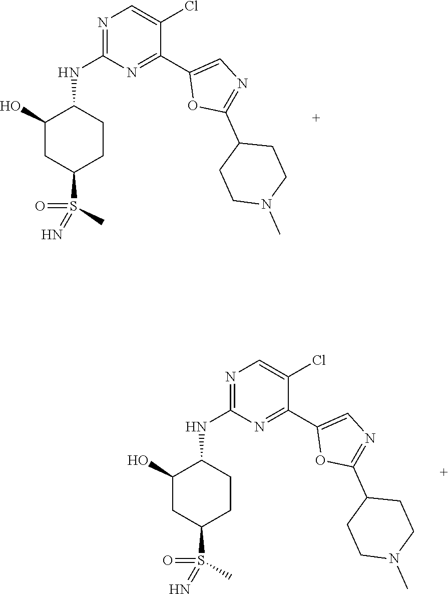 [SMILES: CN1CCC(c2ncc(-c3nc(N[C@@H]4CC[C@@H]([S@](C)(=N)=O)C[C@H]4O)ncc3Cl)o2)CC1 ; CN1CCC(c2ncc(-c3nc(N[C@@H]4CC[C@@H]([S@@](C)(=N)=O)C[C@H]4O)ncc3Cl)o2)CC1]

Step 8. The resulting material was purified by Pre-chiral-HPLC (Column: CHIRALPAK IC, 2×25 cm, 5 μm; Mobile Phase A: Hex(0.5% 2M NH₃-MeOH), Mobile Phase B: IPA:DCM=1:1-HPLC; Flow rate: 20 mL/min; Gradient: 80% B to 80% B in 24 min; Wavelength: 220/254 nm; RT1 (min): 9.813; RT2 (min): 17.926; Sample Solvent: EtOH:DCM=1:1-HPLC; Injection Volume: 1.5 mL; Number Of Runs: 2).

Lyophilization of eluted peak 1, yielded (R)-((1R,3R,4R)-4-((5-chloro-4-(2-(1-methylpiperidin-4-yl) oxazol-5-yl) pyrimidin-2-yl) amino)-3-hydroxycyclohexyl) (imino)(methyl)-λ⁶-sulfanone (I-1035, 24.4 mg, 52.0 mol, 90%, 99.9% Purity) as an off-white solid. m/z (ES⁺) [M+H]⁺=469.10; HPLC tR=0.618 min. ¹H NMR (400 MHz, DMSO-d₆) 8.41 (s, 1H), 8.00 (s, 1H), 7.41 (s, 1H), 4.93 (d, J=4.9 Hz, 1H), 3.59 (s, 3H), 3.01 (t, J=12.4 Hz, 1H), 2.84 (s, 4H), 2.77 (d, J=11.0 Hz, 2H), 2.30 (s, 1H), 2.19 (s, 3H), 2.01 (d, J=15.5 Hz, 6H), 1.78 (q, J=11.6 Hz, 2H), 1.46 (dq, J=23.5, 12.2 Hz, 2H), 1.30 (m, 1H).

Lyophilization of eluted peak 2 yielded (S)-((1R,3R,4R)-4-((5-chloro-4-(2-(1-methylpiperidin-4-yl) oxazol-5-yl)pyrimidin-2-yl)amino)-3-hydroxycyclohexyl)(imino)(methyl)-λ⁶-sulfanone (I-1036, 15.7 mg, 33.3 mol, 87%, 99.6% Purity) as an off-white solid. m/z (ES⁺) [M+H]⁺=469.10; HPLC tR=0.618 min. ¹H NMR (400 MHz, DMSO-d₆) 8.40 (s, 1H), 8.00 (s, 1H), 7.41 (s, 1H), 4.94 (s, 1H), 3.58 (s, 3H), 3.01 (t, J=12.2 Hz, 1H), 2.84 (s, 4H), 2.76 (s, 2H), 2.28 (d, J=12.2 Hz, 1H), 2.19 (s, 3H), 2.01 (d, J=16.9 Hz, 6H), 1.78 (q, J=11.4 Hz, 2H), 1.47 (q, J=16.7, 14.5 Hz, 2H), 1.32 (t, J=12.1 Hz, 1H).

### Example 52

### (3S,4R)-4-((5-chloro-4-(3-(5-(2-hydroxypropan-2-yl)-4-isopropyl-4H-1,2,4-triazol-3-yl)bicyclo[1.1.1]pentan-1-yl)pyrimidin-2-yl)amino)tetrahydro-2H-pyran-3-ol (I-1049)

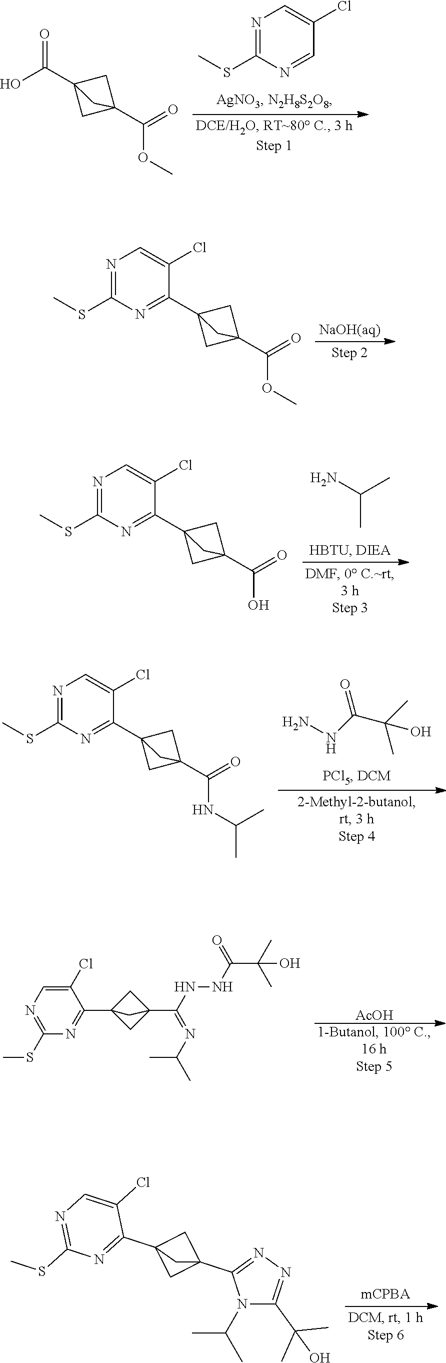

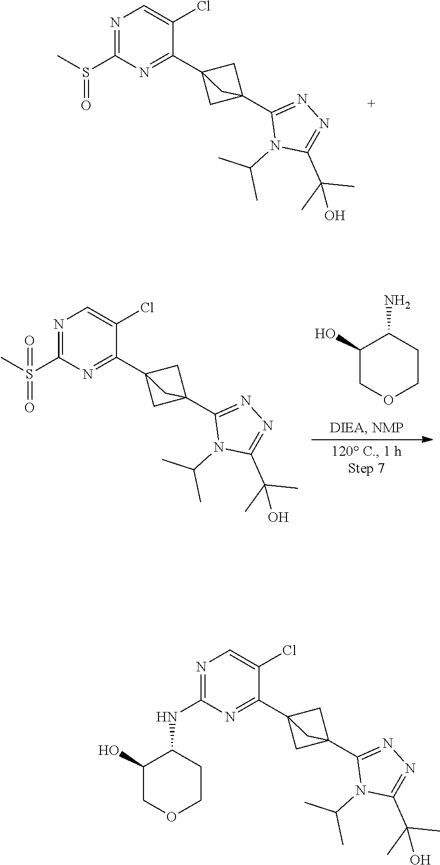

### methyl 3-(5-chloro-2-(methylthio) pyrimidin-4-yl) bicyclo[1.1.1]pentane-1-carboxylate

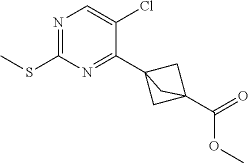 [SMILES: COC(=O)C12CC(c3nc(SC)ncc3Cl)(C1)C2]

Step 1. 5-chloro-2-(methylthio)pyrimidine (3.6 g, 22 mmol) and Ammonium peroxydisulfate (13 g, 56 mmol) were added to a solution of 3-(methoxycarbonyl)bicyclo[1.1.1]pentane-1-carboxylic acid (11 g, 67 mmol) in DCE (96 mL) and water (96 mL). Then silver nitrate (0.76 g, 4.5 mmol) was added at room temperature. The mixture was stirred at 80° C. for 3 h. After cooling, the reaction was extraction with DCM, dried over Na₂SO₄ and evaporated in vacuo. The crude residue was purified by flash (Mobile Phase A: Water (0.1% TFA), Mobile Phase B: ACN; Flow rate: 85 mL/min; Gradient: 75% B to 100% B in 4 min); After solvent evaporation afforded methyl 3-(5-chloro-2-(methylthio) pyrimidin-4-yl) bicyclo[1.1.1]pentane-1-carboxylate (1 g, 4 mmol, 20%) as a brown solid. m/z (ES⁺) [M+H]⁺=285.00; HPLC tR=1.113 min.

### 3-(5-chloro-2-(methylthio)pyrimidin-4-yl)bicyclo[1.1.1]pentane-1-carboxylic acid

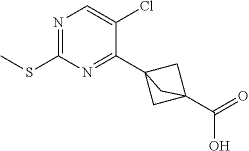 [SMILES: CSc1ncc(Cl)c(C23CC(C(=O)O)(C2)C3)n1]

Step 2. To a solution of methyl 3-(5-chloro-2-(methylthio)pyrimidin-4-yl)bicyclo[1.1.1]pentane-1-carboxylate (1 g, 4 mmol) in MeOH (12 mL), NaOH (0.6 g, 0.01 mol) was added. The mixture was stirred at room temperature for 1 hour. The reaction was worked up by addition of 20 mL water, adjust PH=2 by 3M HCl(aq) and extraction with ethyl acetate, dried over Na₂SO₄. Solvent evaporation afforded 3-(5-chloro-2-(methylthio)pyrimidin-4-yl)bicyclo[1.1.1]pentane-1-carboxylic acid (930 mg, 3.44 mmol, 100%) crude as a dark yellow solid. m/z (ES⁺) [M+H]⁺=271.05; HPLC tR=0.997 min.

### 3-(5-chloro-2-(methylthio)pyrimidin-4-yl)-N-isopropylbicyclo[1.1.1]pentane-1-carboxamide

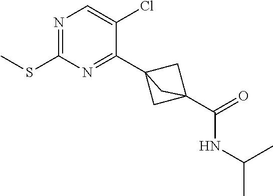 [SMILES: CSc1ncc(Cl)c(C23CC(C(=O)NC(C)C)(C2)C3)n1]

Step 3. To an ice cold solution of 3-(5-chloro-2-(methylthio)pyrimidin-4-yl)bicyclo[1.1.1]pentane-1-carboxylic acid (930 mg, 3.44 mmol) crude, propan-2-amine (609 mg, 10.3 mmol) and DIEA (1.33 g, 10.3 mmol) in DMF (8 mL), HBTU (2.61 g, 6.87 mmol) was added. The mixture was stirred at room temperature for 3 h. The crude residue was purified by flash (Mobile Phase A: Water, Mobile Phase B: ACN; Flow rate: 80 mL/min; Gradient: 45% B to 65% B in 8 min). Solvent evaporation afforded 3-(5-chloro-2-(methylthio)pyrimidin-4-yl)-N-isopropylbicyclo[1.1.1]pentane-1-carboxamide (722 mg, 2.32 mmol, 67.4%) as an off-white solid. m/z (ES⁺) [M+H]⁺=312.00; HPLC tR=0.918 min.

### (E)-N′-((3-(5-chloro-2-(methylthio)pyrimidin-4-yl)bicyclo[1.1.1]pentan-1-yl) (isopropylimino)methyl)-2-hydroxy-2-methylpropanehydrazide

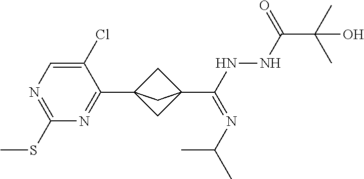 [SMILES: CSc1ncc(Cl)c(C23CC(/C(=N\C(C)C)NNC(=O)C(C)(C)O)(C2)C3)n1]

Step 4. A solution of 3-(5-chloro-2-(methylthio)pyrimidin-4-yl)-N-isopropylbicyclo[1.1.1]pentane-1-carboxa mide (720 mg, 2.31 mmol) in dichloromethane (3 mL) was slowly added to a slurry of pentachloro-15-phosphane (2.40 g, 11.5 mmol) in dichloromethane (4 mL) at 0° C., while keeping the temperature below 10° C. And the mixture was allowed to warm to ambient temperature for over 2 hours. The resulting yellow solution was cooled to 0° C., and a solution of 2-hydroxy-2-methyl propanehydrazide (1.36 g, 11.5 mmol) in 2-Methyl-2-butanol (7 mL) was slowly added while keeping the temperature below 10° C. The reaction was stirred at 30° C. for 1 hour. The reaction was evaporated in vacuo. The crude residue was purified by flash (Mobile Phase A: Water (0.1% TFA), Mobile Phase B: ACN; Flow rate: 60 mL/min; Gradient: 35% B to 55% B in 8 min). Solvent evaporation afforded (E)-N′-((3-(5-chloro-2-(methylthio)pyrimidin-4-yl)bicyclo[1.1.1]pentan-1-yl) (isopropylimino)methyl)-2-hydroxy-2-methylpropanehydrazide (200 mg, 485 mol, 21.0%) as a colorless solid. m/z (ES⁺) [M+H]⁺=412.20; HPLC tR=0.563 min.

### 2-(5-(3-(5-chloro-2-(methylthio)pyrimidin-4-yl)bicyclo[1.1.1]pentan-1-yl)-4-isopropyl-4H-1,2,4-triazol-3-yl)propan-2-ol

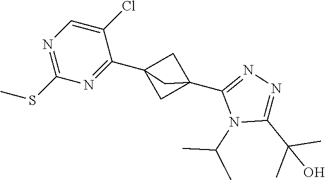 [SMILES: CSc1ncc(Cl)c(C23CC(c4nnc(C(C)(C)O)n4C(C)C)(C2)C3)n1]

Step 5. To a solution of (E)-N′-((3-(5-chloro-2-(methylthio)pyrimidin-4-yl)bicyclo[1.1.1]pentan-1-yl) (isopropylimino)methyl)-2-hydroxy-2-methylpropanehydrazide (200 mg, 485 μmol) in 1-Butanol (3 mL), was added AcOH (87.5 mg, 1.46 mmol). The mixture was heated under 100° C. for 16 h. After cooling, the crude residue was purified by flash (Mobile Phase A: Water (0.1% TFA), Mobile Phase B: ACN; Flow rate: 60 mL/min; Gradient: 35% B to 55% B in 9 min); After solvent evaporation afforded 115 mg crude. The crude product obtained was purified by Prep-TLC (eluted with ¼ petroleum ether/ethyl acetate). Solvent evaporation afforded 2-(5-(3-(5-chloro-2-(methylthio)pyrimidin-4-yl)bicyclo[1.1.1]pentan-1-yl)-4-isopropyl-4H-1,2,4-triazol-3-yl)propan-2-ol (70 mg, 0.18 mmol, 37%) as a colorless solid. m/z (ES⁺) [M+H]⁺=394.15; HPLC tR=0.638 min.

### 2-(5-(3-(5-chloro-2-(methylsulfinyl)pyrimidin-4-yl) bicycle[1.1.1]pentan-1-yl)-4-isopropyl-4H-1,2,4-triazol-3-yl)propan-2-ol &

### 2-(5-(3-(5-chloro-2-(methylsulfonyl)pyrimidin-4-yl)bicyclo[1.1.1]pentan-1-yl)-4-isopropyl-4H-1,2,4-triazol-3-yl)propan-2-ol

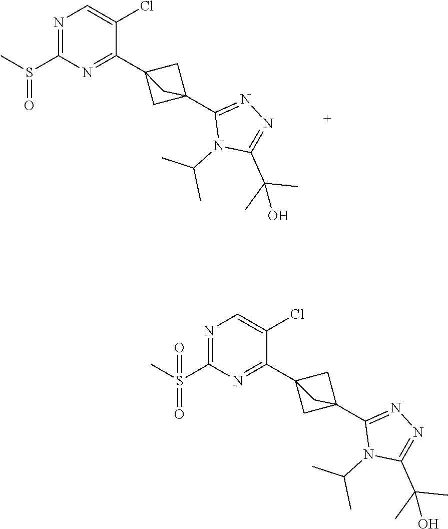

Step 6. To an ice cold solution of 2-(5-(3-(5-chloro-2-(methylthio)pyrimidin-4-yl)bicyclo[1.1.1]pentan-1-yl)-4-isopropyl-4H-1,2,4-triazol-3-yl)propan-2-ol (65 mg, 0.17 mmol) in DCM (1 mL), 3-chloro benzoperoxoic acid (31 mg, 0.18 mmol) was added. The mixture was stirred at room temperature for 1 h. The reaction was worked up by addition of 1 mL water and extraction with DCM, dried over Na₂SO₄. After solvent evaporation afforded 50 mg crude of 2-(5-(3-(5-chloro-2-(methylsulfinyl)pyrimidin-4-yl) bicycle[1.1.1]pentan-1-yl)-4-isopropyl-4H-1,2,4-triazol-3-yl)propan-2-ol and 2-(5-(3-(5-chloro-2-(methylsulfonyl)pyrimidin-4-yl)bicyclo[1.1.1]pentan-1-yl)-4-isopropyl-4H-1,2,4-triazol-3-yl)propan-2-ol. m/z (ES⁺) [M+H]=410.10; HPLC tR=0.563 min & m/z (ES⁺) [M+H]⁺=426.10; HPLC tR=0.655 min.

### (3S,4R)-4-((5-chloro-4-(3-(5-(2-hydroxypropan-2-yl)-4-isopropyl-4H-1,2,4-triazol-3-yl)bicyclo[1.1.1]pentan-1-yl)pyrimidin-2-yl)amino)tetrahydro-2H-pyran-3-ol

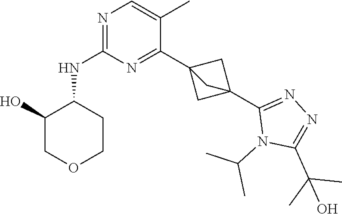 [SMILES: Cc1cnc(N[C@@H]2CCOC[C@H]2O)nc1C12CC(c3nnc(C(C)(C)O)n3C(C)C)(C1)C2]

Step 7. 50 mg crude of 2-(5-(3-(5-chloro-2-(methylsulfinyl)pyrimidin-4-yl)bicycle[1.1.1]pentan-1-yl)-4-isopro pyl-4H-1,2,4-triazol-3-yl)propan-2-ol and 2-(5-(3-(5-chloro-2-(methylsul fonyl)pyrimidin-4-yl)bicycle[1.1.1]pentan-1-yl)-4-isopropyl-4H-1,2,4-triazol-3-yl)propan-2-ol was dissolved in NMP (0.5 mL), (3 S,4R)-4-amino tetrahydro-2H-pyran-3-ol hydrochloride (42 mg, 0.27 mmol) and DIEA (85 mg, 0.11 mL, 6 Eq, 0.66 mmol) were added. The mixture was heated under 120° C. for 1 hour. After cooling, the crude residue was purified by Prep-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 20% B to 45% B in 7 min; Wavelength: 254 nm/220 nm nm; RT1 (min): 6.2); Lyophilization yielded (3S,4R)-4-((5-chloro-4-(3-(5-(2-hydroxypropan-2-yl)-4-isopropyl-4H-1,2,4-triazol-3-yl)bicyclo[1.1.1]pentan-1-yl)pyrimidin-2-yl)amino)tetrahydro-2H-pyran-3-ol (11.6 mg, 25.1 mol, 23%) as an off-white solid. m/z (ES⁺) [M+H]-=463.15; HPLC tR=0.600 min. ¹H NMR (400 MHz, DMSO-d₆) δ 8.23 (s, 1H), 7.34 (d, J=8.0 Hz, 1H), 5.61 (s, 1H), 5.42 (s, 1H), 4.91 (s, 1H), 3.83-3.68 (m, 3H), 3.52-3.44 (m, 1H), 3.29 (d, J=11.6 Hz, 1H), 3.01 (t, J=10.3 Hz, 1H), 2.66 (s, 6H), 1.87 (s, 1H), 1.55 (s, 6H), 1.45 (d, J=7.1 Hz, 7H).

### Example 53

### 3-(5-chloro-2-(((3S,4R)-3-hydroxytetrahydro-2H-pyran-4-yl)amino)pyrimidin-4-yl)-N-isopropylbicyclo[1.1.1]pentane-1-carboxamide (I-1081)

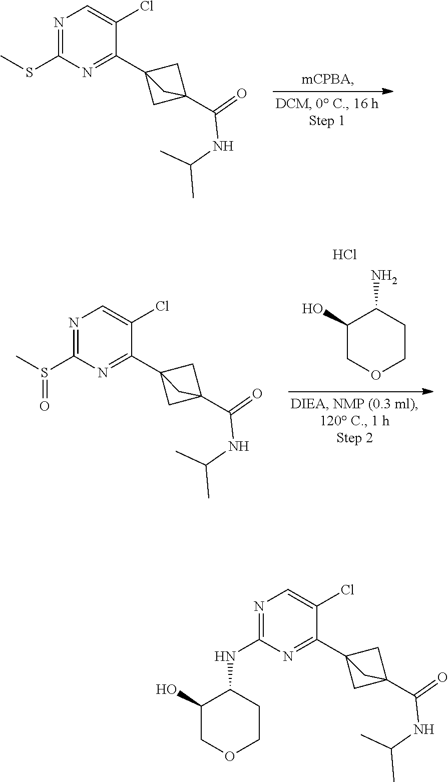 [SMILES: CSc1ncc(Cl)c(C23CC(C(=O)NC(C)C)(C2)C3)n1 ; Cl ; CC(C)NC(=O)C12CC(c3nc(S(C)=O)ncc3Cl)(C1)C2 ; N[C@@H]1CCOC[C@H]1O ; CC(C)NC(=O)C12CC(c3nc(N[C@@H]4CCOC[C@H]4O)ncc3Cl)(C1)C2]

### 3-(5-chloro-2-(methylsulfinyl)pyrimidin-4-yl)-N-isopropylbicyclo[1.1.1]pentane-1-carboxamide

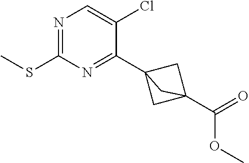 [SMILES: COC(=O)C12CC(c3nc(SC)ncc3Cl)(C1)C2]

Step 1. To a stirred solution of 3-(5-chloro-2-(methylthio)pyrimidin-4-yl)-N-isopropylbicyclo[1.1.1]pentane-1-carboxamide (50 mg, 0.16 mmol) in DCM (2 mL) was added m-CPBA (36 mg, 85% Wt, 0.18 mmol) under 0° C. The reaction was stirred at 0° C. for 1 hour. The reaction mixture was diluted with water (3 mL), and the aqueous phase was extracted with DCM (5 mL) three times. The combined organic layers were washed with brine, dried over sodium sulfate, filtered, and concentrated in vacuo. The residue was purified by Prep-TLC (ACN/EA; ratio: 1/1) to afford 3-(5-chloro-2-(methylsulfinyl)pyrimidin-4-yl)-N-isopropylbicyclo[1.1.1]pentane-1-carboxamide (40 mg, 0.12 mmol, 76%) as an off-white solid. m/z (ES⁺) [M+H]⁻=327.95; HPLC tR=0.550 min.

### 3-(5-chloro-2-(((3S,4R)-3-hydroxytetrahydro-2H-pyran-4-yl)amino)pyrimidin-4-yl)-N-isopropylbicyclo[1.1.1]pentane-1-carboxamide

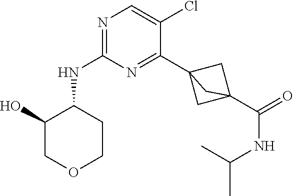 [SMILES: CC(C)NC(=O)C12CC(c3nc(N[C@@H]4CCOC[C@H]4O)ncc3Cl)(C1)C2]

Step 2. A round bottom flask was charged with 3-(5-chloro-2-(methylsulfinyl)pyrimidin-4-yl)-N-isopropylbicyclo[1.1.1]pentane-1-carboxamide (35 mg, 0.11 mmol) in NMP (0.3 mL) was added (3S,4R)-4-aminotetrahydro-2H-pyran-3-ol hydrochloride (49 mg, 0.32 mmol), DIEA (83 mg, 0.64 mmol) and a stirbar. The solution was stirred for 1 hour at 120° C. The mixture was cooled to 25° C. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 12% B to 39% B in 7 min; Wavelength: 254 nm/220 nm nm; RT1 (min): 7.53). Lyophilization yielded 3-(5-chloro-2-(((3S,4R)-3-hydroxytetrahydro-2H-pyran-4-yl)amino)pyrimidin-4-yl)-N-isopropylbicyclo[1.1.1]pentane-1-carboxamide (9.1 mg, 24 μmol, 22%, 99.8% Purity) as an off-white solid. m/z (ES⁺) [M+H]⁺=381.05; HPLC tR=0.659 min. ¹H NMR (400 MHz, DMSO-d₆) 8.18 (s, 1H), 7.65 (d, J=7.9 Hz, 1H), 7.30 (d, J=7.9 Hz, 1H), 4.91 (s, 1H), 3.83 (ddt, J=24.3, 12.4, 6.7 Hz, 4H), 3.48 (s, 1H), 3.29 (s, 1H), 3.00 (t, J=10.4 Hz, 1H), 2.31 (s, 6H), 1.90 (d, J=38.4 Hz, 1H), 1.55-1.37 (m, 1H), 1.06 (d, J=6.6 Hz, 6H).

### Example 54

### (3S,4R)-4-((5-chloro-4-((2-(2-hydroxypropan-2-yl)-1-isopropyl-1H-imidazol-5-yl)ethynyl)pyrimidin-2-yl)amino)tetrahydro-2H-pyran-3-ol (I-1058)

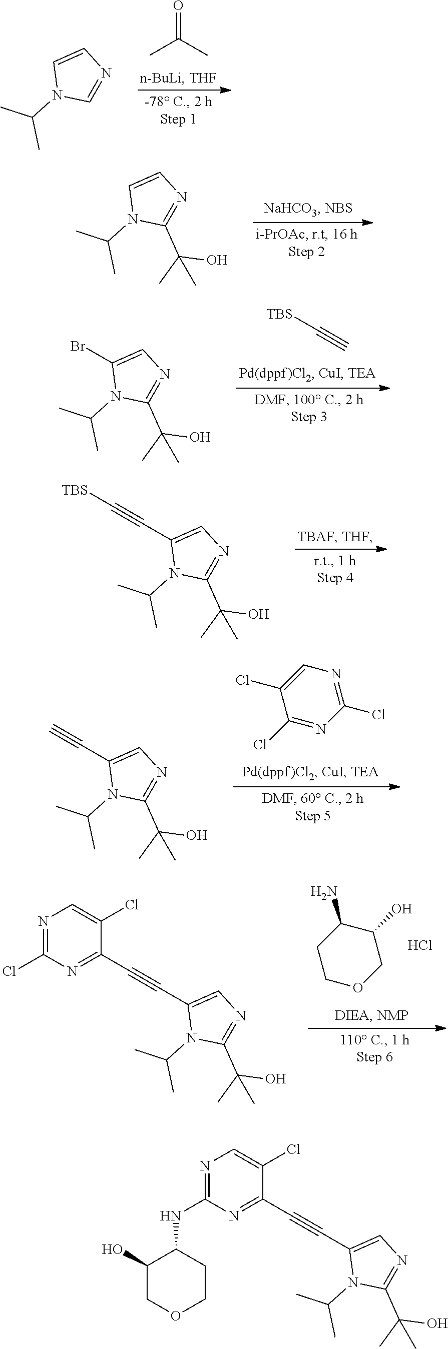

### 2-(1-isopropyl-1H-imidazol-2-yl)propan-2-ol

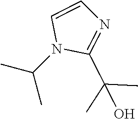

Step 1. To a solution of 1-isopropyl-1H-imidazole (7.9 g, 1 Eq, 72 mmol) in THE (50 mL) was added dropwise n-butyllithium (57 mL, 2 Eq, 2.5 mol/L in n-hexane) at −78° C. under N₂ atmosphere. The reaction mixture was stirred at −78° C. for 1 hour. Then propan-2-one (9.2 g, 2.2 Eq, 0.16 mol) was added dropwise and the mixture was stirred for another 1 hour at −78° C. The reaction was quenched with saturated NH₄C₁(aq.)(1 mL). The crude product was concentrated under vacuum and purified by silica gel chromatography (eluting with PE/EA; ratio: 2/1). The combined organic extracts were concentrated under vacuum to afford 2-(1-isopropyl-1H-imidazol-2-yl)propan-2-ol (7.2 g, 43 mmol, 60%) as a white solid. m/z (ES⁺) [M+H]⁺=169.15; HPLC tR=0.527 min.

### 2-(5-bromo-1-isopropyl-1H-imidazol-2-yl)propan-2-ol

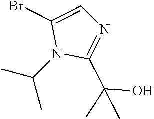

Step 2. To a solution of 2-(1-isopropyl-1H-imidazol-2-yl)propan-2-ol (1.4 g, 1 Eq, 8.3 mmol) in isoamyl acetate (14 mL) was added sodium hydrogen carbonate (0.84 g, 1.2 Eq, 10 mmol) at 25° C. Then 1-bromopyrrolidine-2,5-dione (1.5 g, 1 Eq, 8.3 mmol) was added to above solution in portions. The mixture was stirred for 16 hours at 25° C. The solution was concentrated. The residue was purified by Prep-TLC (PE/EA; ratio: 5/1) to afford 2-(5-bromo-1-isopropyl-1H-imidazol-2-yl)propan-2-ol (560 mg, 2.27 mmol, 27%) as a white solid. m/z (ES⁺) [M+H]⁺=247.05; HPLC tR=0.710 min.

### 2-(5-((tert-butyldimethylsilyl)ethynyl)-1-isopropyl-1H-imidazol-2-yl)propan-2-ol

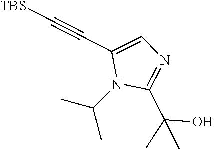

Step 3. A resealable reaction vial was charged with 2-(5-bromo-1-isopropyl-1H-imidazol-2-yl)propan-2-ol (600 mg, 1 Eq, 2.43 mmol), TEA (1.23 g, 1.69 mL, 5 Eq, 12.1 mmol), Pd(dppf)Cl₂ (178 mg, 0.1 Eq, 243 μmol), CuI (46.2 mg, 0.1 Eq, 243 mol), tert-butyl(ethynyl)dimethylsilane (1.70 g, 5 Eq, 12.1 mmol), DMF (10 mL) and a stirbar before being evacuated and purged with nitrogen three times. The mixture was stirred for 2 hours at 100° C. The solution was purified by reverse flash chromatography with the following conditions (mobile phase, MeCN in water, 70% to 90% gradient in 20 min; detector, UV 254 nm) to afford 2-(5-((tert-butyldimethylsilyl)ethynyl)-1-isopropyl-1H-imidazol-2-yl)propan-2-ol (465 mg, 1.52 mmol, 62.5%) as a yellow oil. m/z (ES⁺) [M+H]⁺=307.20; HPLC tR=1.132 min.

### 2-(5-ethynyl-1-isopropyl-1H-imidazol-2-yl)propan-2-ol

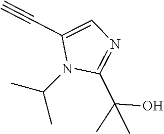

Step 4. To a solution of 2-(5-((tert-butyldimethylsilyl)ethynyl)-1-isopropyl-1H-imidazol-2-yl)propan-2-ol (460 mg, 1 Eq, 1.50 mmol) in THF (2 mL) was added tetrabutylammonium fluoride (471 mg, 1.2 Eq, 1.80 mmol). The solution was stirred for 1 hour at 25° C. The mixture was purified by reverse flash chromatography with the following conditions: (120 g, mobile phase, MeCN in water, 30% to 60% gradient in 15 min; detector, UV 254 nm) to afford 2-(5-ethynyl-1-isopropyl-1H-imidazol-2-yl)propan-2-ol (170 mg, 884 μmol, 58.9%) as a yellow oil. m/z (ES⁺) [M+H]⁺=193.15; HPLC tR=0.673 min.

### 2-(5-((2,5-dichloropyrimidin-4-yl)ethynyl)-1-isopropyl-1H-imidazol-2-yl)propan-2-ol

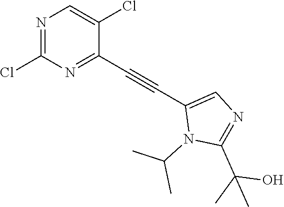 [SMILES: CC(C)n1c(C#Cc2nc(Cl)ncc2Cl)cnc1C(C)(C)O]

Step 5. A resealable reaction vial was charged with 2-(5-ethynyl-1-isopropyl-1H-imidazol-2-yl)propan-2-ol (160 mg, 1 Eq, 832 mol), triethylamine (253 mg, 3 Eq, 2.50 mmol), copper(I) iodide (15.8 mg, 0.1 Eq, 83.2 mol), 2,4,5-trichloropyrimidine (229 mg, 1.5 Eq, 1.25 mmol), Pd(dppf)Cl₂ (60.9 mg, 0.1 Eq, 83.2 μmol), DMF (8 mL) and a stirbar before being evacuated and purged with nitrogen three times. The mixture was stirred for 2 hours at 60° C. The solution was purified by reverse flash chromatography with the following conditions: column, C18 silica gel; mobile phase, MeCN in water, 20% to 55% gradient in 20 min; detector, UV 254 nm. Concentration in vacuo resulted in 2-(5-((2,5-dichloropyrimidin-4-yl)ethynyl)-1-isopropyl-1H-imidazol-2-yl)propan-2-ol (108 mg, 318 μmol, 38.3%) as a yellow oil. m/z (ES⁺) [M+H]⁺=338.95; HPLC tR=0.600 min.

### (3S,4R)-4-((5-chloro-4-((2-(2-hydroxypropan-2-yl)-1-isopropyl-1H-imidazol-5-yl)ethynyl)pyrimidin-2-yl)amino)tetrahydro-2H-pyran-3-ol

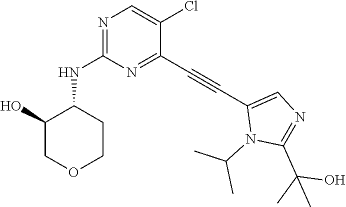 [SMILES: CC(C)n1c(C#Cc2nc(N[C@@H]3CCOC[C@H]3O)ncc2Cl)cnc1C(C)(C)O]

Step 6. To a solution of 2-(5-((2,5-dichloropyrimidin-4-yl)ethynyl)-1-isopropyl-1H-imidazol-2-yl)propan-2-ol (100 mg, 1 Eq, 295 μmol) in NMP (1 mL) were added (3S,4R)-4-aminotetrahydro-2H-pyran-3-ol hydrochloride (54.3 mg, 1.2 Eq, 354 μmol) and N-ethyl-N-isopropylpropan-2-amine (114 mg, 3 Eq, 884 μmol) at 25° C. The solution was stirred for 1 hour at 110° C. The resulting crude material was purified by Prep-HPLC (Column: XBridge Shield RP18 OBD Column, 30×150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 20% B to 43% B in 7 min; Wavelength: 254 nm/220 nm nm; RT1 (min): 6.65; Number Of Runs: 1). Lyophilization yielded (3S,4R)-4-((5-chloro-4-((2-(2-hydroxypropan-2-yl)-1-isopropyl-1H-imidazol-5-yl)ethynyl)pyrimidin-2-yl)amino)tetrahydro-2H-pyran-3-ol (39.5 mg, 94.1 μmol, 31.9%) as a white solid. m/z (ES⁺) [M+H]⁺=420.15; HPLC tR=0.878 min. ¹H NMR (400 MHz, DMSO-d₆) 8.40 (s, 1H), 7.53 (s, 1H), 7.43 (s, 1H), 5.61 (s, 1H), 5.57-5.50 (m, 1H), 4.93 (d, J=5.3 Hz, 1H), 3.80 (d, J=9.5 Hz, 3H), 3.45 (s, 1H), 3.29 (s, 1H), 3.03 (t, J=10.4 Hz, 1H), 1.89 (d, J=13.1 Hz, 1H), 1.62 (d, J=6.8 Hz, 6H), 1.54 (s, 6H), 1.46 (d, J=12.3 Hz, 1H).

### Example 55

### (3S,4R)-4-((5-chloro-4-(2-((R)-1-methylpyrrolidin-3-yl) thiazol-5-yl) pyrimidin-2-yl) amino) tetrahydro-2H-pyran-3-ol (I-1000), &

### (3S,4R)-4-((5-chloro-4-(2-((S)-1-methylpyrrolidin-3-yl) thiazol-5-yl) pyrimidin-2-yl) amino) tetrahydro-2H-pyran-3-ol (I-1002)

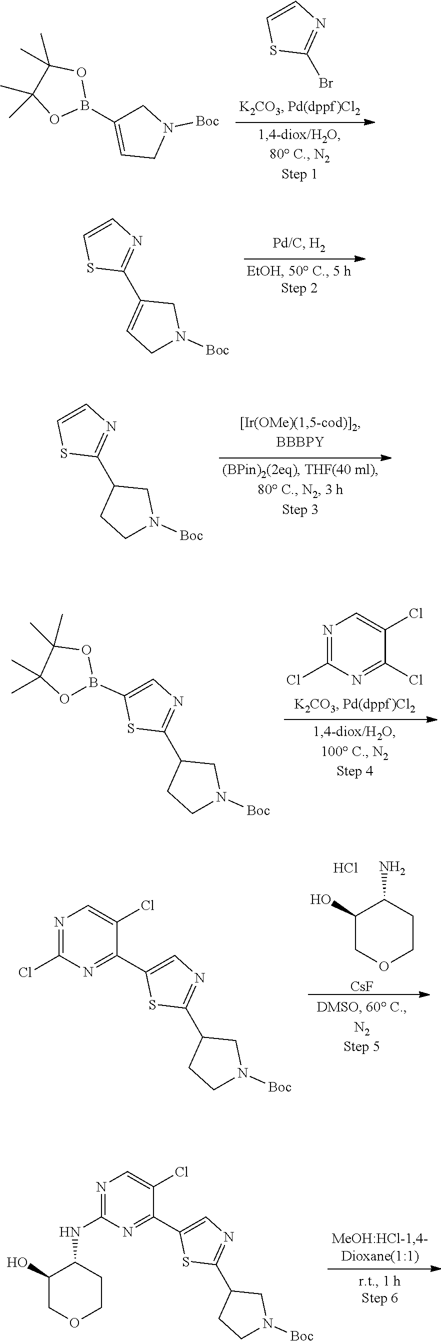

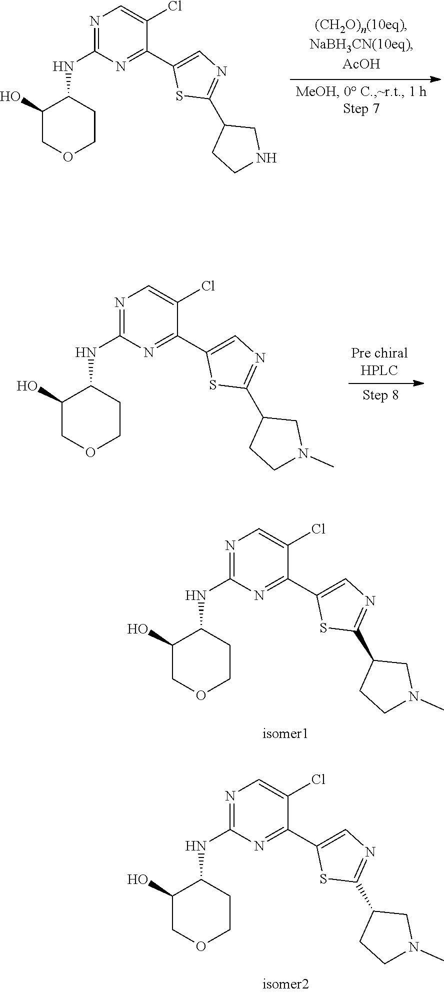 [SMILES: O[C@@H]1COCC[C@H]1Nc1ncc(Cl)c(-c2cnc(C3CCNC3)s2)n1 ; CN1CCC(c2ncc(-c3nc(N[C@@H]4CCOC[C@H]4O)ncc3Cl)s2)C1 ; CN1CC[C@@H](c2ncc(-c3nc(N[C@@H]4CCOC[C@H]4O)ncc3Cl)s2)C1 ; CN1CC[C@H](c2ncc(-c3nc(N[C@@H]4CCOC[C@H]4O)ncc3Cl)s2)C1]

### tert-butyl 3-(thiazol-2-yl)-2,5-dihydro-1H-pyrrole-1-carboxylate

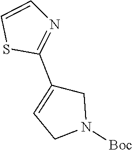 [SMILES: CC(C)(C)OC(=O)N1CC=C(c2nccs2)C1]

Step 1. A resealable reaction vial was charged with 2-bromothiazole (2 g, 0.01 mol) in 1,4-dioxane (12 mL) and water (3.0 mL), tert-butyl 3-(4,4,5,5-tetramethyl-1,3,2-dioxaborolan-2-yl)-2,5-dihydro-1H-pyrrole-1-carboxylate (3 g, 0.01 mol), K₂CO₃ (4 g, 0.03 mol), and a stirbar before being evacuated and purged with nitrogen three times. PdCl₂(dppf)-CH₂Cl₂ adduct (2 g, 2 mmol) was added at r.t. The resulting mixture was stirred at 80° C. for 2 hours under nitrogen atmosphere. The reaction mixture was diluted with water (30 mL), and the aqueous phase was extracted with EA (50 mL) three times. The combined organic layers were washed with brine, dried over sodium sulfate, filtered, and concentrated in vacuo. The residue was purified by reverse flash chromatography with the following conditions: column, C18 silica gel; mobile phase, ACN in water, 10% to 100% gradient in 20 min; detector, UV 254 nm. Concentration in vacuo to afford tert-butyl 3-(thiazol-2-yl)-2,5-dihydro-1H-pyrrole-1-carboxylate (1.8 g, 6.8 mmol, 70%, 95% Purity) as a dark yellow solid. m/z (ES⁺) [M+H-tBu]⁺=197.05; HPLC tR=0.730 min.

### tert-butyl 3-(thiazol-2-yl) pyrrolidine-1-carboxylate

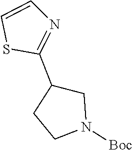 [SMILES: CC(C)(C)OC(=O)N1CCC(c2nccs2)C1]

Step 2. A solution of tert-butyl 3-(thiazol-2-yl)-2,5-dihydro-1H-pyrrole-1-carboxylate (1.7 g, 6.7 mmol) in EtOH (5 mL) was bubbled nitrogen through the reaction mixture for 3 times, and then added Pd/C (0.72 g, 6.7 mmol) under N₂ atmosphere, then bubbled H₂ for 3 times. The mixture was stirred at 50° C. for 5 hours under H₂. The reaction mixture was filtered, and the and the filtrate was concentrated in vacuo. The crude product was purified by silica gel chromatography (10 g column; eluting with PE/EA; ratio: 30/1). Concentration in vacuo resulted in tert-butyl 3-(thiazol-2-yl) pyrrolidine-1-carboxylate (1.3517 g, 4.45 mmol, 66%, 83.7% purity) as a yellow oil. m/z (ES⁺) [M+H]⁺=255.00; HPLC tR=0.861 min.

### tert-butyl 3-(5-(4,4,5,5-tetramethyl-1,3,2-dioxaborolan-2-yl) thiazol-2-yl) pyrrolidine-1-carboxylate

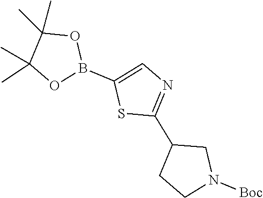 [SMILES: CC(C)(C)OC(=O)N1CCC(c2ncc(B3OC(C)(C)C(C)(C)O3)s2)C1]

Step 3. To a solution of tert-butyl 3-(thiazol-2-yl) pyrrolidine-1-carboxylate (800 mg, 3.15 mmol) in THE (40 mL) was added 4,4,4′,4′,5,5,5′,5′-octamethyl-2,2′-bi(1,3,2-dioxaborolane) (1.41 g, 5.54 mmol), dimethyliridium(1+) methanolate-(1Z,5Z)-1,5-cyclooctadiene (208 mg, 315 mol) and 4,4′-di-tert-butyl-2,2′-bipyridyl (338 mg, 1.26 mmol). The mixture was stirred at 80° C. under nitrogen atmosphere for 3 hours. The reaction mixture was concentrated in vacuo. The residue was purified by Prep-TLC (EA/PE; ratio: 2/3) to afford tert-butyl 3-(5-(4,4,5,5-tetramethyl-1,3,2-dioxaborolan-2-yl) thiazol-2-yl) pyrrolidine-1-carboxylate (1.1 g, 1.7 mmol, 55%, 59.5% Purity) as a yellow oil. m/z (ES⁺) [M+H]⁺=381.10; HPLC tR=0.778 min.

### tert-butyl 3-(5-(2,5-dichloropyrimidin-4-yl) thiazol-2-yl) pyrrolidine-1-carboxylate

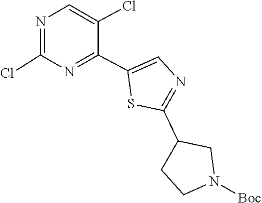 [SMILES: CC(C)(C)OC(=O)N1CCC(c2ncc(-c3nc(Cl)ncc3Cl)s2)C1]

Step 4. To a mixture of tert-butyl 3-(5-(4,4,5,5-tetramethyl-1,3,2-dioxaborolan-2-yl) thiazol-2-yl) pyrrolidine-1-carboxylate (1.1 g, 2.9 mmol) in 1,4-Dioxane (8.9 mL) was added 2,4,5-trichloropyrimidine (0.64 g, 3.5 mmol), then PdCl₂(dppf) (0.42 g, 0.58 mmol), K₂CO₃ (1.6 g, 12 mmol) and H₂O (0.89 mL) under nitrogen atmosphere. The mixture was stirred at 100° C. for 2 hours. The mixture was purified by reverse flash chromatography with the following conditions: column, C18 silica gel; mobile phase, MeCN in water, 0% to 100% gradient in 30 min; detector, UV 254 nm. Concentration in vacuo resulted in tert-butyl 3-(5-(2,5-dichloropyrimidin-4-yl) thiazol-2-yl) pyrrolidine-1-carboxylate (188 mg, 14%, 86.2% Purity) as a yellow oil. m/z (ES⁺) [M+H]⁺=401.00; HPLC tR=1.095 min.

### tert-butyl 3-(5-(5-chloro-2-(((3S,4R)-3-hydroxytetrahydro-2H-pyran-4-yl) amino) pyrimidin-4-yl) thiazol-2-yl) pyrrolidine-1-carboxylate

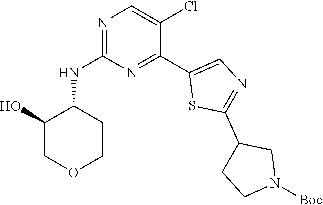 [SMILES: CC(C)(C)OC(=O)N1CCC(c2ncc(-c3nc(N[C@@H]4CCOC[C@H]4O)ncc3Cl)s2)C1]

Step 5. To a solution of tert-butyl 3-(5-(2,5-dichloropyrimidin-4-yl) thiazol-2-yl) pyrrolidine-1-carboxylate (178 mg, 444 mol) in DMSO (3 mL) were added (3S,4R)-4-aminotetrahydro-2H-pyran-3-ol hydrochloride (102 mg, 665 mol), CsF (337 mg, 2.22 mmol) under nitrogen atmosphere. The mixture was stirred at 60° C. for 3 hours. The residue was purified by reverse flash chromatography with the following conditions: column, C18 silica gel; mobile phase, MeCN in water, 0% to 100% gradient in 15 min; detector, UV 254 nm. And then concentrated in vacuo resulted in tert-butyl 3-(5-(5-chloro-2-(((3S,4R)-3-hydroxytetrahydro-2H-pyran-4-yl) amino) pyrimidin-4-yl) thiazol-2-yl) pyrrolidine-1-carboxylate (143 mg, 297 μmol, 66.9%) as a yellow solid. m/z (ES⁺) [M+H]⁺=482.15; HPLC tR=0.875 min.

### (3S,4R)-4-((5-chloro-4-(2-(pyrrolidin-3-yl) thiazol-5-yl) pyrimidin-2-yl) amino) tetrahydro-2H-pyran-3-ol

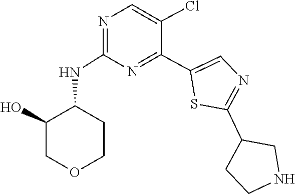 [SMILES: O[C@@H]1COCC[C@H]1Nc1ncc(Cl)c(-c2cnc(C3CCNC3)s2)n1]

Step 6. A solution of tert-butyl 3-(5-(5-chloro-2-(((3S,4R)-3-hydroxytetrahydro-2H-pyran-4-yl) amino) pyrimidin-4-yl) thiazol-2-yl) pyrrolidine-1-carboxylate (133 mg, 276 mol) in MeOH (1.5 mL) and hydrogen chloride in 1,4-dioxane (1.5 mL, 4M), then was stirred at 25° C. for 1 hour. Concentration in vacuo resulted in (3S,4R)-4-((5-chloro-4-(2-(pyrrolidin-3-yl) thiazol-5-yl) pyrimidin-2-yl) amino) tetrahydro-2H-pyran-3-ol (105 mg, 274 mol, 99.3%, 99.7% Purity) as a white solid. m/z (ES⁺) [M+H]⁺=382.05; HPLC tR=0.545 min.

### (3S,4R)-4-((5-chloro-4-(2-(1-methylpyrrolidin-3-yl) thiazol-5-yl) pyrimidin-2-yl) amino) tetrahydro-2H-pyran-3-ol

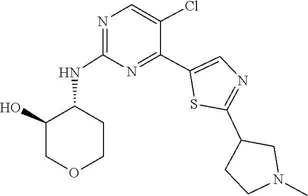 [SMILES: CN1CCC(c2ncc(-c3nc(N[C@@H]4CCOC[C@H]4O)ncc3Cl)s2)C1]

Step 7. To a mixture of (3S,4R)-4-((5-chloro-4-(2-(pyrrolidin-3-yl) thiazol-5-yl) pyrimidin-2-yl) amino) tetrahydro-2H-pyran-3-ol (124 mg, 325 mol), AcOH (19.5 mg, 18.6 μL, 325 μmol) and CH₂O (48.7 mg, 1.62 mmol) in MeOH (5 mL) was added Sodium cyanoborohydride (102 mg, 1.62 mmol) at 0° C. under nitrogen atmosphere. The mixture was stirred for 1 hour at 25° C. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 12% B to 39% B in 7 min; Wavelength: 254 nm/220 nm nm; RT1 (min): 7.73). Lyophilization yielded (3S,4R)-4-((5-chloro-4-(2-(1-methylpyrrolidin-3-yl) thiazol-5-yl) pyrimidin-2-yl) amino) tetrahydro-2H-pyran-3-ol (64 mg, 0.16 mmol, 50%, 99.9% Purity) as a yellow solid. m/z (ES⁺) [M+H]⁺=396.10; HPLC tR=0.637 min.

### (3S,4R)-4-((5-chloro-4-(2-((R)-1-methylpyrrolidin-3-yl) thiazol-5-yl) pyrimidin-2-yl) amino) tetrahydro-2H-pyran-3-ol &

### (3S,4R)-4-((5-chloro-4-(2-((S)-1-methylpyrrolidin-3-yl) thiazol-5-yl) pyrimidin-2-yl) amino) tetrahydro-2H-pyran-3-ol

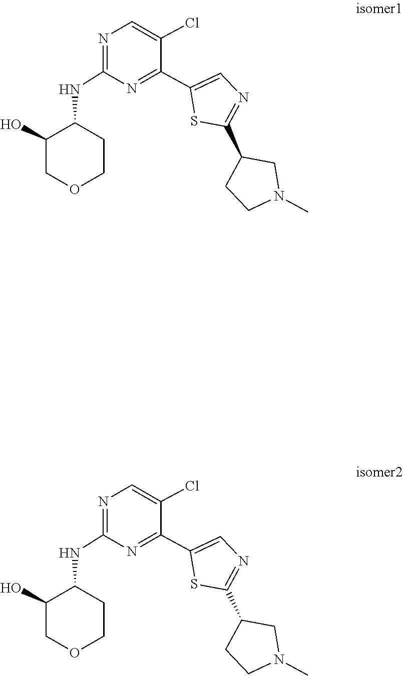 [SMILES: CN1CC[C@@H](c2ncc(-c3nc(N[C@@H]4CCOC[C@H]4O)ncc3Cl)s2)C1 ; CN1CC[C@H](c2ncc(-c3nc(N[C@@H]4CCOC[C@H]4O)ncc3Cl)s2)C1]

Step 8. The resulting material was purified by Pre-chiral-HPLC (Column: CHIRALPAK IE, 2×25 cm, 5 μm; Mobile Phase A: MTBE (0.5% 2M NH₃-MeOH), Mobile Phase B: IPA: DCM=1:1-HPLC; Flow rate: 20 mL/min; Gradient: 15% B to 15% B in 26 min; Wavelength: 220/254 nm; RT1 (min): 19.138; RT2 (min): 22.138; Sample Solvent: EtOH:DCM=1:1-HPLC; Injection Volume: 0.7 mL; Number Of Runs: 7).

Isomer 1: Lyophilization of eluted peak 1 yielded (3S,4R)-4-((5-chloro-4-(2-((R)-1-methylpyrrolidin-3-yl) thiazol-5-yl) pyrimidin-2-yl) amino) tetrahydro-2H-pyran-3-ol (I-1000, 21.2 mg, 66%, 99.74% Purity) as an off-white solid. m/z (ES⁺) [M+H]⁺=396.05; HPLC tR=0.698 min. ¹H NMR (400 MHz, DMSO-d₆) 8.59 (s, 1H), 8.41 (s, 1H), 7.47 (s, 1H), 4.94 (d, J=5.4 Hz, 1H), 3.81 (d, J=12.4 Hz, 4H), 3.50 (s, 1H), 3.34 (s, 2H), 3.04 (s, 1H), 2.84 (s, 1H), 2.71 (dd, J=9.2, 5.1 Hz, 2H), 2.32 (s, 4H), 1.99 (s, 2H), 1.47 (q, J=11.6, 11.2 Hz, 1H).

Isomer 2: Lyophilization of eluted peak 2 yielded (3S,4R)-4-((5-chloro-4-(2-((S)-1-methylpyrrolidin-3-yl) thiazol-5-yl) pyrimidin-2-yl) amino) tetrahydro-2H-pyran-3-ol (I-1002, 20.9 mg, 52.6 μmol, 65%, 99.70% Purity) as an off-white solid. m/z (ES⁺) [M+H]⁺=396.05; HPLC tR=0.698 min. ¹H NMR (400 MHz, DMSO-d₆) 8.59 (s, 1H), 8.41 (s, 1H), 7.48 (s, 1H), 4.94 (d, J=5.3 Hz, 1H), 3.92-3.66 (m, 4H), 3.50 (s, 1H), 3.34 (s, 2H) 3.04 (s, 1H), 2.85 (s, 1H), 2.73 (dd, J=9.3, 5.2 Hz, 2H), 2.33 (s, 4H), 1.95 (d, J=36.8 Hz, 2H), 1.47 (q, J=14.8, 12.8 Hz, 1H).

### Example 56

### (3S,4R)-4-((5-chloro-4-(3-((R)-1-methylpiperidin-3-yl)-1,2,4-thiadiazol-5-yl)pyridin-2-yl)amino)tetrahydro-2H-pyran-3-ol (I-1076) &

### (3S,4R)-4-((5-chloro-4-(3-((S)-1-methylpiperidin-3-yl)-1,2,4-thiadiazol-5-yl)pyridin-2-yl)amino)tetrahydro-2H-pyran-3-ol (I-1074)

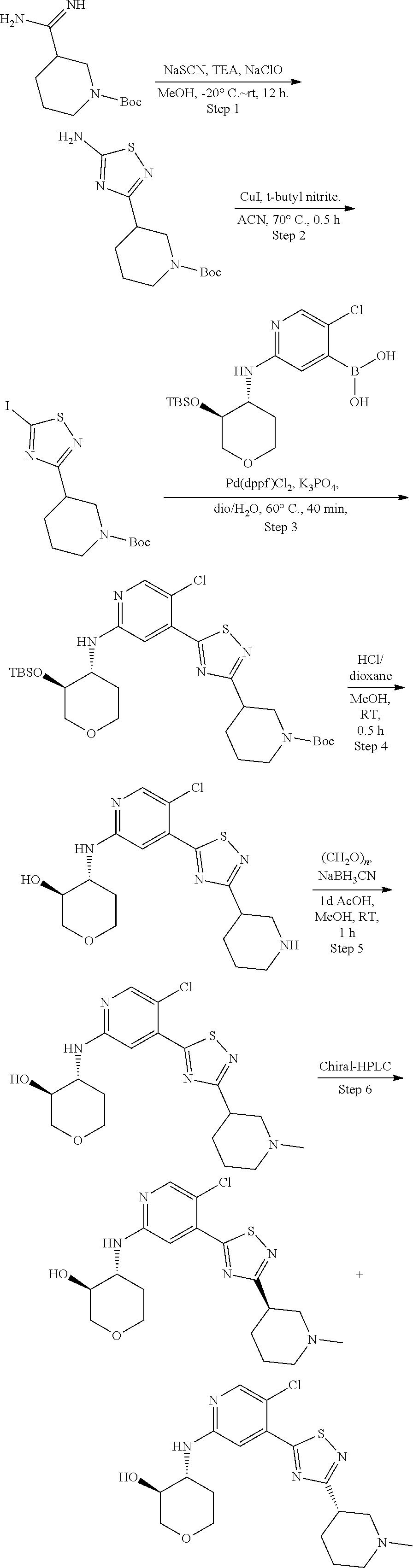

### tert-butyl 3-(5-amino-1,2,4-thiadiazol-3-yl)piperidine-1-carboxylate

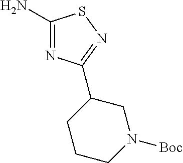 [SMILES: CC(C)(C)OC(=O)N1CCCC(c2nsc(N)n2)C1]

Step 1. The mixture of sodium thiocyanate (372 mg, 5.03 mmol) in MeOH (20 ml) was stirred for 5 min at −20° C. Then TEA (469 mg, 4.64 mmol) and tert-butyl 3-carbamimidoylpiperidine-1-carboxylate (880 mg, 3.87 mmol) were added in portions at −20° C. under nitrogen atmosphere. The mixture was stirred at −20° C. for 45 min. Then TEA (391 mg, 3.87 mmol) and sodium hypochlorite (345 mg, 4.26 mmol) were added and the resulting reaction was stirred for another 1 hour at −20° C. At last the reaction was stirred for 12 hours at rt. The reaction was evaporated in vacuo and worked up by addition of 10 ml water and extraction with ethyl acetate, dried over Na₂SO₄, filtered, the filtrate was concentrated. Solvent evaporation afforded tert-butyl 3-(5-amino-1,2,4-thiadiazol-3-yl)piperidine-1-carboxylate (400 mg) crude as a colorless solid. m/z (ES⁺) [M+H]⁺=285.10; HPLC tR=0.655 min.

### tert-butyl 3-(5-iodo-1,2,4-thiadiazol-3-yl)piperidine-1-carboxylate

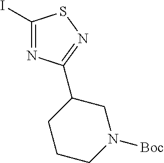 [SMILES: CC(C)(C)OC(=O)N1CCCC(c2nsc(I)n2)C1]

Step 2. The solution of tert-butyl 3-(5-amino-1,2,4-thiadiazol-3-yl)piperidine-1-carboxylate (390 mg, 1.37 mmol) crude and CuI (1.04 g, 5.49 mmol) in acetonitrile (5 mL) was bubbling nitrogen through the reaction mixture, then tert-Butyl nitrite (283 mg, 2.74 mmol) was added dropwise to the reaction. The mixture was stirred at 70° C. for 0.5 hour. After cooling, the solution was purified by flash (Mobile Phase A: Water, Mobile Phase B: ACN; Flow rate: 70 mL/min; Gradient: 70% B to 100% B in 5 min). Solvent evaporation afforded tert-butyl 3-(5-iodo-1,2,4-thiadiazol-3-yl)piperidine-1-carboxylate (33 mg, 83 μmol, 6.1%) as a tan solid. m/z (ES⁺) [M+H]⁺=396.00; HPLC tR=1.113 min.

### tert-butyl 3-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperid ine-1-carboxylate

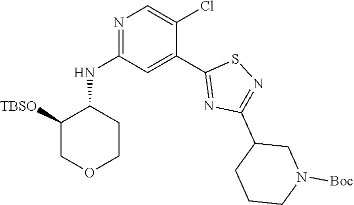 [SMILES: CC(C)(C)OC(=O)N1CCCC(c2nsc(-c3cc(N[C@@H]4CCOC[C@H]4O[Si](C)(C)C(C)(C)C)ncc3Cl)n2)C1]

Step 3. (2-(((3S,4R)-3-((tert-butyldimethylsilyl)oxy)tetrahydro-2H-pyran-4-yl)amino)-5-chloropyridin-4-yl) boronic acid (29 mg, 76 mol) and K₃PO₄ (32 mg, 0.15 mmol) were added to a solution of tert-butyl 3-(5-iodo-1,2,4-thiadiazol-3-yl)piperidine-1-carboxylate (20 mg, 51 mol) in dioxane/H₂O=3:1 (1 ml). After bubbling nitrogen through the reaction mixture for 1 minutes, Pd(dppf)Cl₂ (6.2 mg, 7.6 μmol) was added. The reaction mixture was heated at 60° C. for 40 min with vigorous stirring. The solution was worked up by addition of 3 ml water and extracted with ethyl acetate (3 ml) three times, dried over Na₂SO₄ and evaporated in vacuo. The crude product obtained was purified by Prep-TLC (eluted with a 4/1 petroleum ether/ethyl acetate). Solvent evaporation afforded tert-butyl 3-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)piperid ine-1-carboxylate (28 mg, 46 μmol, 91%) as a yellow solid. m/z (ES⁺) [M+H]⁺=610.40; HPLC tR=1.197 min.

### (3S,4R)-4-((5-chloro-4-(3-(piperidin-3-yl)-1,2,4-thiadiazol-5-yl)pyridin-2-yl)amino)tetrahydro-2H-pyran-3-ol

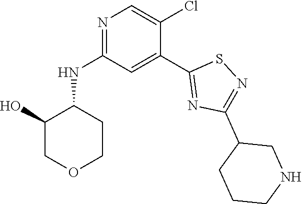 [SMILES: O[C@@H]1COCC[C@H]1Nc1cc(-c2nc(C3CCCNC3)ns2)c(Cl)cn1]

Step 4. To a solution of tert-butyl 3-(5-(2-(((3S,4R)-3-((tert-butyldimethylsilyl)oxy)tetrahydro-2H-pyran-4-yl) amino)-5-chloropyridin-4-yl)-1,2,4-thiadiazol-3-yl)piperidine-1-carboxylate (26 mg, 43 mol) in MeOH (0.3 ml), HCl/Dioxane (0.2 ml, 0.4 molar) was added. The mixture was stirred at RT for 0.5 hour. Most was product in LCMS. The reaction was concentrated and adjusted to PH=8 by NaHCO₃ aqueous solution. Solvent evaporation afforded (3S,4R)-4-((5-chloro-4-(3-(piperidin-3-yl)-1,2,4-thiadiazol-5-yl)pyridin-2-yl)amino)tetrahydro-2H-pyran-3-ol (40 mg, 0.10 mmol) crude as a yellow solid. m/z (ES⁺) [M+H]=396.20; HPLC tR=0.513 min.

### (3S,4R)-4-((5-chloro-4-(3-(1-methylpiperidin-3-yl)-1,2,4-thiadiazol-5-yl)pyridin-2-yl)amino) tetrahydro-2H-pyran-3-ol

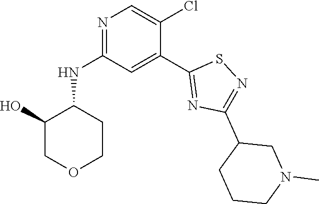 [SMILES: CN1CCCC(c2nsc(-c3cc(N[C@@H]4CCOC[C@H]4O)ncc3Cl)n2)C1]

Step 5. To a solution of (3S,4R)-4-((5-chloro-4-(3-(piperidin-3-yl)-1,2,4-thiadiazol-5-yl)pyridin-2-yl)amino) tetrahydro-2H-pyran-3-ol (30 mg, 76 mol) crude in MeOH (1.5 ml), formaldehyde (18 mg, 0.61 mmol) was added. Then the mixture was cooled to 0° C. and sodium cyanotrihydroborate (24 mg, 0.38 mmol) and 1 drop of AcOH were added. The reaction was stirred at room temperature for 1 hour. The reaction was filtered to remove the filter cake. The filtrate was purified by Prep-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 27% B to 50% B in 7 min; Wavelength: 254 nm/220 nm nm; RT1 (min): 6.77). Solvent evaporation afforded (3S,4R)-4-((5-chloro-4-(3-(1-methylpiperidin-3-yl)-1,2,4-thiadiazol-5-yl)pyridin-2-yl)amino) tetrahydro-2H-pyran-3-ol (10 mg, 24 μmol, 32%) as a yellow solid. [M+H]⁺=410.05; HPLC tR=0.898 min.

### (3S,4R)-4-((5-chloro-4-(3-((R)-1-methylpiperidin-3-yl)-1,2,4-thiadiazol-5-yl)pyridin-2-yl)amino)tetrahydro-2H-pyran-3-ol &

### (3S,4R)-4-((5-chloro-4-(3-((S)-1-methylpiperidin-3-yl)-1,2,4-thiadiazol-5-yl)pyridin-2-yl)amino)tetrahydro-2H-pyran-3-ol

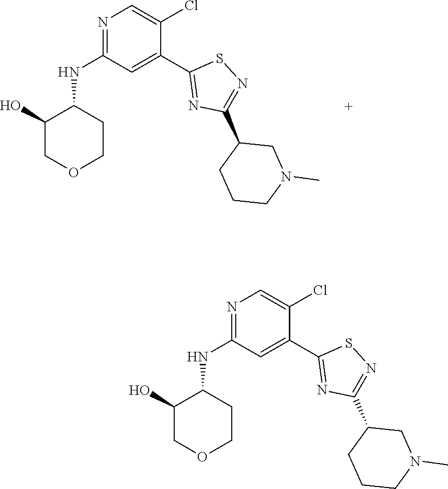 [SMILES: CN1CCC[C@@H](c2nsc(-c3cc(N[C@@H]4CCOC[C@H]4O)ncc3Cl)n2)C1 ; CN1CCC[C@H](c2nsc(-c3cc(N[C@@H]4CCOC[C@H]4O)ncc3Cl)n2)C1]

Step 6. 3S,4R)-4-((5-chloro-4-(3-(1-methylpiperidin-3-yl)-1,2,4-thiadiazol-5-yl)pyridin-2-yl)amino)tetrahydro-2H-pyran-3-ol (10 mg, 24 mol) was purified by Chiral-HPLC (Column: CHIRALPAK IC, 2×25 cm, 5 μm; Mobile Phase A: Hex(0.5% 2M NH₃-MeOH), Mobile Phase B: EtOH:DCM=1:1-HPLC, Flow rate: 20 mL/min; Gradient: 40% B to 40% B in 7 min, Wavelength: 220/254 nm; RT1 (min): 5.199; RT2 (min): 5.959; Sample Solvent: EtOH:DCM=1:1-HPLC; Injection Volume: 1.0 mL).

Lyophilization of eluted peak 1 yielded (3S,4R)-4-((5-chloro-4-(3-((R)-1-methylpiperidin-3-yl)-1,2,4-thiadiazol-5-yl)pyridin-2-yl)amino)tetrahydro-2H-pyran-3-ol (2 mg, mol, 40%) as a yellow solid. [M+H]⁺=410.05; HPLC tR=0.978 min. ¹H NMR (400 MHz, DMSO-d₆) δ 8.25 (s, 1H), 7.53 (s, 1H), 7.25 (d, J=7.6 Hz, 1H), 4.98 (d, J=5.2 Hz, 1H), 3.80 (td, J=10.9, 4.3 Hz, 3H), 3.46-3.34 (m, 2H), 3.31 (s, 1H), 3.15-3.04 (m, 2H), 2.75 (d, J=11.2 Hz, 1H), 2.27 (d, J=24.3 Hz, 1H), 2.22 (s, 3H), 2.11 (d, J=11.0 Hz, 1H), 2.05-1.89 (m, 2H), 1.80-1.72 (m, 1H), 1.61 (q, J=12.4 Hz, 2H), 1.46-1.31 (m, 1H).

Lyophilization of eluted peak 2 yielded (3S,4R)-4-((5-chloro-4-(3-((S)-1-methylpiperidin-3-yl)-1,2,4-thiadiazol-5-yl)pyridin-2-yl)amino)tetrahydro-2H-pyran-3-ol (2.2 mg, 5.4 μmol, 40%) as a yellow solid. [M+H]⁺=410.10; HPLC tR=0.978 min. ¹H NMR (400 MHz, DMSO-d₆) δ 8.25 (s, 1H), 7.53 (s, 1H), 7.25 (d, J=7.6 Hz, 1H), 4.98 (d, J=5.3 Hz, 1H), 3.81 (dt, J=14.5, 7.4 Hz, 3H), 3.44-3.34 (m, 2H), 3.31 (s, 1H), 3.14-3.04 (m, 2H), 2.76 (s, 1H), 2.24 (s, 4H), 2.11 (d, J=10.5 Hz, 1H), 2.06-1.89 (m, 2H), 1.80-1.71 (m, 1H), 1.60 (q, J=12.4 Hz, 2H), 1.47-1.33 (m, 1H).

### Example 57

### (3S,4R)-4-((5-fluoro-4-(2-(1-methylpiperidin-4-yl)thiazol-5-yl)pyrimidin-2-yl)amino)tetrahydro-2H-pyran-3-ol (I-1042)

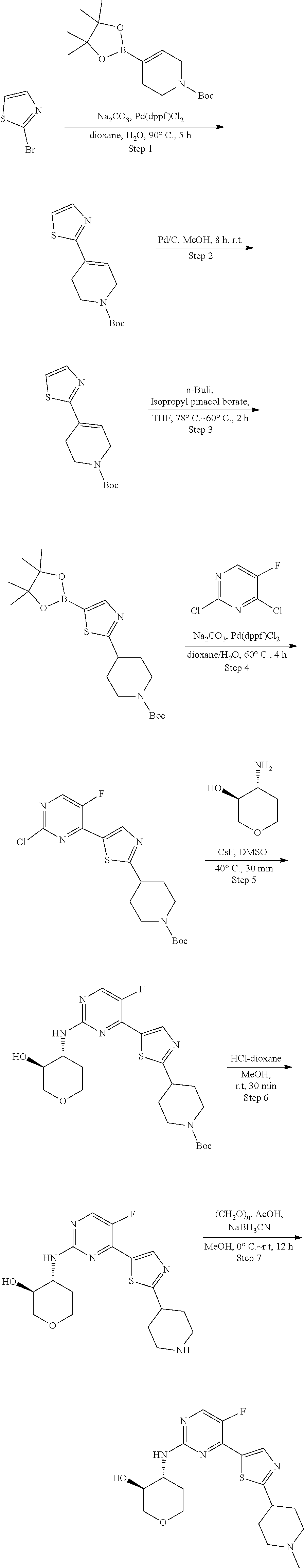

### tert-butyl 4-(thiazol-2-yl)-3,6-dihydropyridine-1(2H)-carboxylate

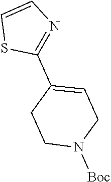 [SMILES: CC(C)(C)OC(=O)N1CC=C(c2nccs2)CC1]

Step 1. A round bottom flask was charged with tert-butyl 4-(4,4,5,5-tetramethyl-1,3,2-dioxaborolan-2-yl)-3,6-dihydropyridine-1(2H)-carboxylate (21 g, 67 mmol), 2-bromothiazole (10 g, 61 mmol), PdCl₂(dppf) (4.5 g, 6.1 mmol), Na₂CO₃ (19 g, 180 mmol), 1,4-dioxane (160 mL) and H₂O (40 mL) and a stirbar before being evacuated and purged with nitrogen three times, and the mixture was stirred for 5 hours at 90° C. The reaction mixture was diluted with water (200 mL), and the aqueous phase was extracted with EA (100 mL) three times. The combined organic layers were washed with brine, dried over sodium sulfate, filtered, and concentrated in vacuo. The residue was purified by silica gel chromatography eluting with PE/EA; ratio: 4/1. Concentration in vacuo resulted in tert-butyl 4-(thiazol-2-yl)-3,6-dihydropyridine-1(2H)-carboxylate (7.5 g, 46%) as a yellow oil. m/z (ES⁺) [M+H]⁺=267.05; HPLC tR=0.948 min.

### tert-butyl 4-(thiazol-2-yl)piperidine-1-carboxylate

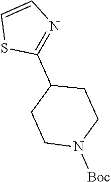 [SMILES: CC(C)(C)OC(=O)N1CCC(c2nccs2)CC1]

Step 2. A stirred mixture of tert-butyl 4-(thiazol-2-yl)-3,6-dihydropyridine-1(2H)-carboxylate (7.5 g, 28 mmol) and Pd/C (0.60 g, 5.6 mmol) in MeOH (75 mL) was treated with H₂ for 8 hours at r.t. The reaction mixture was filtered (through a pad of Celite*), the pad was washed with MeOH, and the filtrate was concentrated in vacuo to get the tert-butyl 4-(thiazol-2-yl)piperidine-1-carboxylate (6.7 g, 89%) as a yellow oil. m/z (ES⁺) [M+H]⁻=269.10; HPLC tR=0.705 min.

### tert-butyl 4-(5-(4,4,5,5-tetramethyl-1,3,2-dioxaborolan-2-yl)thiazol-2-yl)piperidine-1-carboxylate

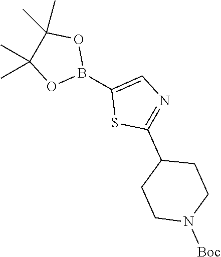 [SMILES: CC(C)(C)OC(=O)N1CCC(c2ncc(B3OC(C)(C)C(C)(C)O3)s2)CC1]

Step 3. The solution of tert-butyl 4-(thiazol-2-yl)piperidine-1-carboxylate (2.8 g, 10 mmol) in THE (28 mL) was bubbling nitrogen through the reaction mixture for 30 minutes and cooled to −78° C., butyllithium (26 mmol, 1.9 mL) was added dropwise to the reaction. The mixture was stirred at −78° C. for 1 hour. Then 2-isopropoxy-4,4,5,5-tetramethyl-1,3,2-dioxaborolane (5.8 g, 31 mmol) was added at −78° C.~−60° C. for 2 hours. The reaction was then quenched by the addition of 5 mL of water. The solution was purified by reverse flash chromatography with the following conditions: column, C18 silica gel; mobile phase, MeCN in water, 40% to 80% gradient in 30 min; detector, UV 254 nm. Concentration in vacuo resulted in tert-butyl 4-(5-(4,4,5,5-tetramethyl-1,3,2-dioxaborolan-2-yl)thiazol-2-yl)piperidine-1-carboxylate (3.01 g, 73%) as a yellow oil. m/z (ES⁺) [M+H]>=395.15; HPLC tR=0.920 min.

### tert-butyl 4-(5-(2-chloro-5-fluoropyrimidin-4-yl)thiazol-2-yl)piperidine-1-carboxylate

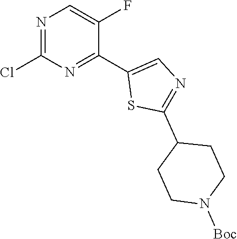 [SMILES: CC(C)(C)OC(=O)N1CCC(c2ncc(-c3nc(Cl)ncc3F)s2)CC1]

Step 4. A resealable reaction vial was charged with tert-butyl 4-(5-(4,4,5,5-tetramethyl-1,3,2-dioxaborolan-2-yl)thiazol-2-yl)piperidine-1-carboxylate (600 mg, 1.52 mmol), 2,4-dichloro-5-fluoropyrimidine (305 mg, 1.83 mmol), Na₂CO₃ (484 mg, 4.56 mmol), PdCl₂(dppf) (111 mg, 0.15 mmol), 1, 4-Dioxane (4.5 mL) and H₂O (1.1 mL) and a stirbar before being evacuated and purged with nitrogen three times, and the mixture was stirred for 4 hour at 60° C. The solution was purified by reverse flash chromatography with the following conditions: column, C18 silica gel; mobile phase, MeCN in water, 50% to 100% gradient in 20 min; detector, UV 254 nm. Concentration in vacuo resulted in tert-butyl 4-(5-(2-chloro-5-fluoropyrimidin-4-yl)thiazol-2-yl)piperidine-1-carboxylate (382 mg, 59%) as a yellow solid. m/z (ES⁺) [M+H]⁺=399.20; HPLC tR=1.013 min.

### tert-butyl 4-(5-(5-fluoro-2-(((3S,4R)-3-hydroxytetrahydro-2H-pyran-4-yl)amino)pyrimidin-4-yl)thiazol-2-yl)piperidine-1-carboxylate

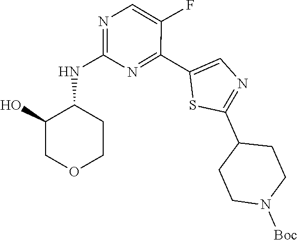 [SMILES: CC(C)(C)OC(=O)N1CCC(c2ncc(-c3nc(N[C@@H]4CCOC[C@H]4O)ncc3F)s2)CC1]

Step 5. A resealable reaction vial was charged with tert-butyl 4-(5-(2-chloro-5-fluoropyrimidin-4-yl)thiazol-2-yl)piperidine-1-carboxylate (252 mg, 0.63 mmol), (3S,4R)-4-aminotetrahydro-2H-pyran-3-ol (74.0 mg, 1.63 mmol), CsF (480 mg, 3.16 mmol), DMSO (2.5 mL) and a stirbar before being evacuated and purged with nitrogen three times, and the mixture was stirred for 30 min at 40° C. The solution was purified by reverse flash chromatography with the following conditions: column, C18 silica gel, mobile phase, MeCN in water, 60% to 80% gradient in 20 min; detector, UV 254 nm. Concentration in vacuo resulted in tert-butyl 4-(5-(5-fluoro-2-(((3S,4R)-3-hydroxytetrahydro-2H-pyran-4-yl)amino)pyrimidin-4-yl)thiazol-2-yl)piperidine-1-carboxylate (260.8 mg, 84%) as a yellow solid. m/z (ES⁺) [M+H]⁺=480.15; HPLC tR=0.878 min.

### (3S,4R)-4-((5-fluoro-4-(2-(piperidin-4-yl)thiazol-5-yl)pyrimidin-2-yl)amino)tetrahydro-2H-pyran-3-ol

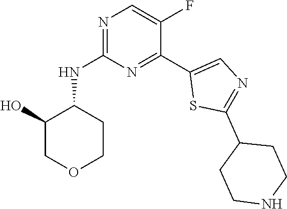 [SMILES: O[C@@H]1COCC[C@H]1Nc1ncc(F)c(-c2cnc(C3CCNCC3)s2)n1]

Step 6. To a mixture of tert-butyl 4-(5-(5-fluoro-2-(((3S,4R)-3-hydroxytetrahydro-2H-pyran-4-yl)amino)pyrimidin-4-yl)thiazol-2-yl)piperidine-1-carboxylate (200 mg, 0.42 mmol) in MeOH (0.5 mL) was added hydrogen chloride in dioxane (2 mL, 50 mmol) dropwise at 25° C. under nitrogen atmosphere. The mixture was stirred for 30 min at 25° C. The solution was concentrated and the pH value of the residue was adjusted to 6~7 with aq.NaHCO₃. The solvent was evaporated in vacuo to give (3S,4R)-4-((5-fluoro-4-(2-(piperidin-4-yl)thiazol-5-yl)pyrimidin-2-yl)amino)tetrahydro-2H-pyran-3-ol (158 mg, 99.8%, crude) as a white solid. m/z (ES⁺) [M+H]⁺=380.10; HPLC tR=0.438 min.

### (3S,4R)-4-((5-fluoro-4-(2-(1-methylpiperidin-4-yl)thiazol-5-yl)pyrimidin-2-yl)amino)tetrahydro-2H-pyran-3-ol

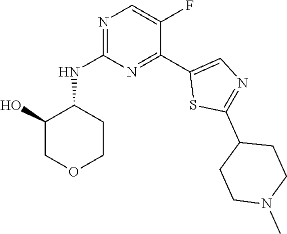 [SMILES: CN1CCC(c2ncc(-c3nc(N[C@@H]4CCOC[C@H]4O)ncc3F)s2)CC1]

Step 7. A mixture of (3S,4R)-4-((5-fluoro-4-(2-(piperidin-4-yl)thiazol-5-yl)pyrimidin-2-yl)amino)tetrahydro-2H-pyran-3-ol (200 mg, 0.53 mmol) and Paraformaldehyde (79.1 mg, 2.64 mmol) in MeOH (10 mL) was stirred for 30 min at 0° C. under nitrogen atmosphere. Then sodium cyanoborohydride (99.4 mg, 1.58 mmol) was added in portions. The mixture was warmed up to rt and stirred for 12 h at r.t. The reaction was quenched with H₂O, the resulting mixture was filtered, the filter cake was washed with MeOH (30 ml), the filtrate was concentrated under reduced pressure. The residue was purified by C18 chromatography (120 g, eluted with MeCN in water; ratio: 0-100%) to afford crude product (200 mg). The crude product (200 mg) was purified by Prep-HPLC with the following conditions (Column: XBridge Prep OBD C18 Column, 30×150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 12% B to 37% B in 8 min; Wavelength: 220 nm nm; RT1 (min): 7.53). Concentration afforded (3S,4R)-4-((5-fluoro-4-(2-(1-methylpiperidin-4-yl)thiazol-5-yl)pyrimidin-2-yl)amino)tetrahydro-2H-pyran-3-ol (59.6 mg, 28.6%) as a white solid. m/z (ES⁺) [M+H]⁺=394.10; HPLC tR=1.478 min. ¹H NMR (400 MHz, DMSO-d₆) δ 8.44 (d, J=3.0 Hz, 1H), 8.32 (d, J=2.0 Hz, 1H), 7.22 (d, J=7.8 Hz, 1H), 4.92 (d, J=5.3 Hz, 1H), 3.86-3.77 (m, 2H), 3.72 (s, 1H), 3.49 (tt, J=9.8, 5.1 Hz, 1H), 3.31 (s, 1H), 3.08-2.95 (m, 2H), 2.83 (d, J=11.8 Hz, 2H), 2.19 (s, 3H), 2.08-1.89 (m, 5H), 1.80-1.69 (m, 2H), 1.45 (qd, J=12.0, 4.6 Hz, 1H).

### Example 58

### (3S,4R)-4-((5-chloro-4-(1-methyl-2-(tetrahydro-2H-pyran-4-yl)-1H-imidazol-5-yl)pyrimidin-2-yl)amino)tetrahydro-2H-pyran-3-ol (I-1079)

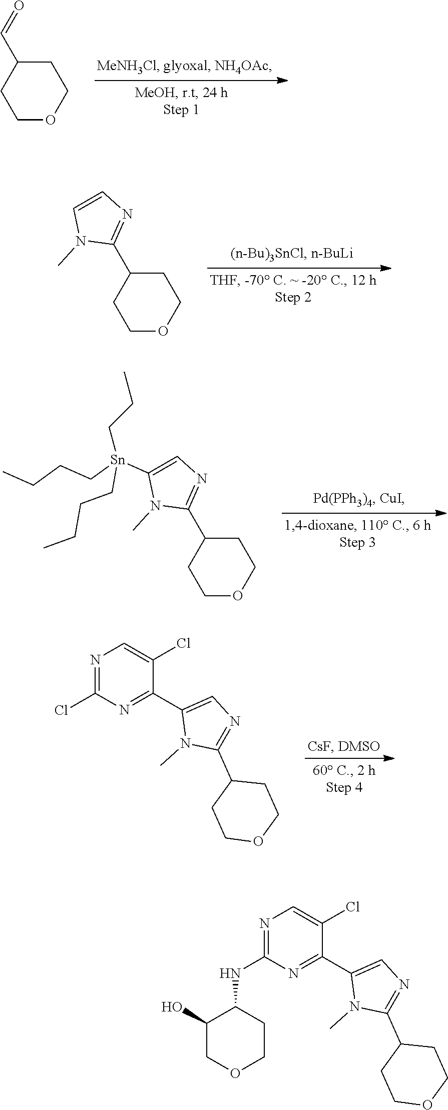

### 1-methyl-2-(tetrahydro-2H-pyran-4-yl)-1H-imidazole

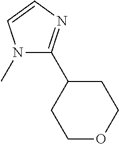

Step 1. A resealable reaction vial was charged with tetrahydro-2H-pyran-4-carbaldehyde (10 g, 88 mmol), Methylamine hydrochloride (7.1 g, 0.11 mol), Ammonium acetate (6.8 g, 6.2 mL, 88 mmol), Glyoxal (5.1 g, 4.0 mL, 88 mmol), MeOH (100 mL) and a stirbar before being evacuated and purged with nitrogen three times, and the mixture was stirred for 24 hours at 25° C. The solvent was removed by distillation under vacuum. The residue was purified by silica gel chromatography (eluting with DCM/MeOH; ratio: 20/1). Concentration in vacuo resulted in 1-methyl-2-(tetrahydro-2H-pyran-4-yl)-1H-imidazole (4.3 g, 24%) as a yellow solid. m/z (ES⁺) [M+H]⁺=167.10; HPLC tR=0.472 min.

### 1-methyl-2-(tetrahydro-2H-pyran-4-yl)-5-(tributylstannyl)-1H-imidazole

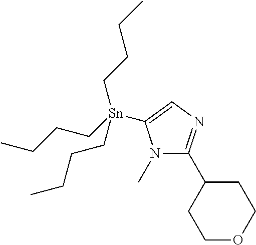 [SMILES: CCC[CH2][Sn]([CH2]CCC)([CH2]CCC)[c]1cnc(C2CCOCC2)n1C]

Step 2. The solution of 1-methyl-2-(tetrahydro-2H-pyran-4-yl)-1H-imidazole (600 mg, 3.61 mmol) in THF (6 mL) was bubbled nitrogen for 3 minutes and cooled to −70° C., butyllithium (1.4 mL, 18.0 mmol) was added dropwise to the reaction. The mixture was stirred at −70° C. for 1 hour. Then tributyltin chloride (7.05 g, 21.7 mmol) was added and the solution was warmed to −20° C. for 12 hours gradually. The reaction was then quenched by the addition of 2 mL of water. The residue was purified by reverse flash chromatography with the following conditions: column, C18 silica gel; mobile phase, MeCN in water, 50% to 100% gradient in 30 min; detector, UV 220 nm. Concentration in vacuo resulted in 1-methyl-2-(tetrahydro-2H-pyran-4-yl)-5-(tributylstannyl)-1H-imidazole (1.07 g, 51%) as a yellow oily liquid. m/z (ES⁺) [M+H]⁺=457.00; HPLC tR=0.750 min.

### 2,5-dichloro-4-(1-methyl-2-(tetrahydro-2H-pyran-4-yl)-1H-imidazol-5-yl)pyrimidine

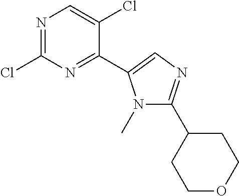 [SMILES: Cn1c(-c2nc(Cl)ncc2Cl)cnc1C1CCOCC1]

Step 3. A resealable reaction vial was charged with 1-methyl-2-(tetrahydro-2H-pyran-4-yl)-5-(tributylstannyl)-1H-imidazole (500 mg, 1.10 mmol) in 1,4-Dioxane (5 mL), CuI (20.9 mg, 0.11 mmol), 2,4,5-trichloropyrimidine (242 mg, 1.32 mmol) and a stirbar before being evacuated and purged with nitrogen three times. Pd(Ph₃P)₄ (127 mg, 0.11 mmol) was added at r.t. The resulting mixture was stirred at 110° C. for 6 hours under nitrogen atmosphere. The reaction mixture was diluted with water (20 mL), and the aqueous phase was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 30% to 60% gradient in 20 min; detector, UV 254 nm. Concentration in vacuo resulted in 2,5-dichloro-4-(1-methyl-2-(tetrahydro-2H-pyran-4-yl)-1H-imidazol-5-yl)pyrimidine (332 mg, 76%) as a yellow liquid. m/z (ES⁺) [M+H]⁺=313.05; HPLC tR=0.472 min.

### (3S,4R)-4-((5-chloro-4-(1-methyl-2-(tetrahydro-2H-pyran-4-yl)-1H-imidazol-5-yl)pyrimidin-2-yl)amino)tetrahydro-2H-pyran-3-ol

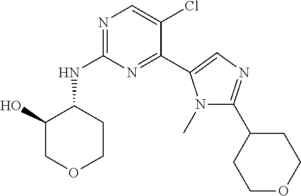 [SMILES: Cn1c(-c2nc(N[C@@H]3CCOC[C@H]3O)ncc2Cl)cnc1C1CCOCC1]

Step 4. A resealable reaction vial was charged with 2,5-dichloro-4-(1-methyl-2-(tetrahydro-2H-pyran-4-yl)-1H-imidazol-5-yl)pyrimidine (100 mg, 0.32 mmol), (3S,4R)-4-aminotetrahydro-2H-pyran-3-ol (37.4 mg, 0.32 mmol), CsF (243 mg, 1.60 mmol), DMSO (1 mL) and a stirbar before being evacuated and purged with nitrogen three times, and the mixture was stirred for 2 hours at 60° C. The resulting crude material was purified by Pre-HPLC (Column: XBridge Prep OBD C18 Column, 30×150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 10% B to 32% B in 7 min; Wavelength: 254 nm/220 nm nm; RT1 (min): 7.55). Lyophilization yielded (3S,4R)-4-((5-chloro-4-(1-methyl-2-(tetrahydro-2H-pyran-4-yl)-1H-imidazol-5-yl)pyrimidin-2-yl)amino)tetrahydro-2H-pyran-3-ol (35.5 mg, 28.1%) as a white solid. m/z (ES⁺) [M+H]⁺=394.10; HPLC tR=0.778 min. ¹H NMR (400 MHz, DMSO-d₆) δ 8.34 (s, 1H), 7.62 (s, 1H), 7.36 (d, J=8.3 Hz, 1H), 4.94 (d, J=5.2 Hz, 1H), 3.94 (dt, J=11.2, 3.2 Hz, 2H), 3.84 (s, 6H), 3.52-3.42 (m, 3H), 3.33-3.27 (m, 1H), 3.15 (td, J=9.8, 4.7 Hz, 1H), 3.02 (dd, J=11.1, 9.7 Hz, 1H), 1.92-1.73 (m, 5H), 1.48 (q, J=11.3 Hz, 1H).

### Example 59

### (3S,4R)-4-((4-(3-(4-amino-4-methylpiperidin-1-yl)-1,2,4-thiadiazol-5-yl)-5-chloropyridin-2-yl)amino) tetrahydro-2H-pyran-3-ol (I-1027)

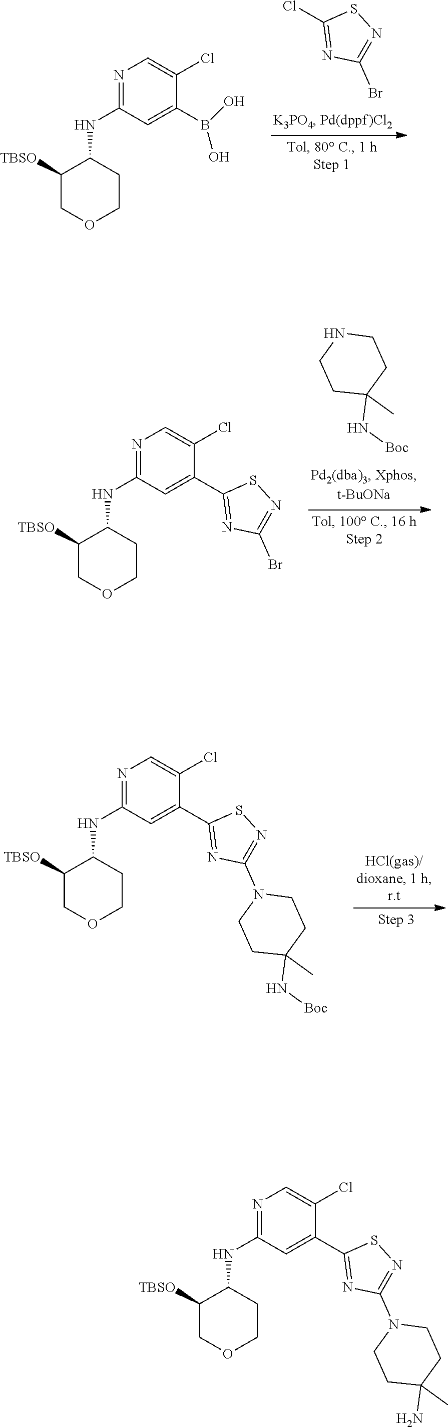 [SMILES: CC(C)(C)[Si](C)(C)O[C@@H]1COCC[C@H]1Nc1cc(B(O)O)c(Cl)cn1 ; Clc1nc(Br)ns1 ; CC(C)(C)[Si](C)(C)O[C@@H]1COCC[C@H]1Nc1cc(-c2nc(Br)ns2)c(Cl)cn1 ; CC1(NC(=O)OC(C)(C)C)CCNCC1 ; CC1(NC(=O)OC(C)(C)C)CCN(c2nsc(-c3cc(N[C@@H]4CCOC[C@H]4O[Si](C)(C)C(C)(C)C)ncc3Cl)n2)CC1 ; CC1(N)CCN(c2nsc(-c3cc(N[C@@H]4CCOC[C@H]4O[Si](C)(C)C(C)(C)C)ncc3Cl)n2)CC1]

### 4-(3-bromo-1,2,4-thiadiazol-5-yl)-N-((3S,4R)-3-((tert-butyldimethylsilyl)oxy)tetrahydro-2H-pyran-4-yl)-5-chloropyridin-2-amine

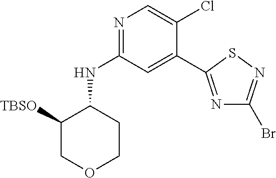 [SMILES: CC(C)(C)[Si](C)(C)O[C@@H]1COCC[C@H]1Nc1cc(-c2nc(Br)ns2)c(Cl)cn1]

Step 1. To a solution of (2-(((3S,4R)-3-((tert-butyldimethylsilyl)oxy)tetrahydro-2H-pyran-4-yl)amino)-5-chloropyridin-4-yl)boronic acid (3 g, 8 mmol) and 3-bromo-5-chloro-1,2,4-thiadiazole (2 g, 8 mmol) in Toluene (50 mL), Potassium phosphate, tribasic (5 g, 20 mmol) and PdCl₂(dppf)-CH₂Cl₂adduct (0.6 g, 0.8 mmol) were added. After stirring for 1 hour at 80° C. under a nitrogen atmosphere, the solution was concentrated under reduced pressure. The residue was purified by reversed-phase flash chromatography with the following conditions: column, C18 silica gel; mobile phase, MeCN in Water, 10% to 100% gradient in 10 min; detector, UV 254 nm to afford 4-(3-bromo-1,2,4-thiadiazol-5-yl)-N-((3S,4R)-3-((tert-butyldimethylsilyl)oxy)tetrahydro-2H-pyran-4-yl)-5-chloropyridin-2-amine (2.7 g, 60%) as a brown solid. m/z (ES⁺) [M+H]⁺=505.10; HPLC tR=1.155 min.

### tert-butyl (1-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)-4-methylpiperidin-4-yl)carbamate

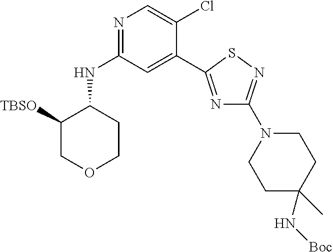 [SMILES: CC1(NC(=O)OC(C)(C)C)CCN(c2nsc(-c3cc(N[C@@H]4CCOC[C@H]4O[Si](C)(C)C(C)(C)C)ncc3Cl)n2)CC1]

Step 2. To a solution of 4-(3-bromo-1,2,4-thiadiazol-5-yl)-N-((3S,4R)-3-((tert-butyldimethylsilyl)oxy) tetrahydro-2H-pyran-4-yl)-5-chloropyridin-2-amine (210 mg, 0.415 mmol) and tert-butyl (4-methylpiperidin-4-yl)carbamate (89.0 mg, 0.415 mmol) in toluene (2 mL), 2-(Dicyclohexylphosphanyl)-2′,4′,6′-tris(isopropyl)biphenyl (19.8 mg, 0.0415 mmol) sodium 2-methylpropan-2-olate (120 mg, 1.25 mmol), and Pd₂(dba)₃ (38.0 mg, 0.0415 mmol) were added. After stirring for 16 hours at 100° C. under a nitrogen atmosphere. The solution was concentrated under reduced pressure. The residue was purified by reversed-phase flash chromatography with the following conditions: column, C18 silica gel; mobile phase, MeCN in Water, 10% to 100% gradient in 10 min; detector, UV 254 nm to afford tert-butyl (1-(5-(2-(((3S,4R)-3-((tert-butyldimethylsilyl)oxy)tetrahydro-2H-pyran-4-yl)amino)-5-chloropyridin-4-yl)-1,2,4-thiadiazol-3-yl)-4-methylpiperidin-4-yl)carbamate (190 mg, 71.6%) as a yellow solid. m/z (ES⁺) [M+H]⁺=639.45; HPLC tR=1.172 min.

### (3S,4R)-4-((4-(3-(4-amino-4-methylpiperidin-1-yl)-1,2,4-thiadiazol-5-yl)-5-chloropyridin-2-yl)amino) tetrahydro-2H-pyran-3-ol

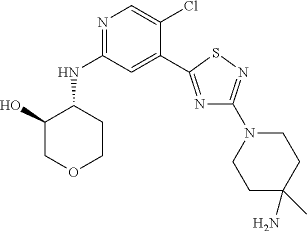 [SMILES: CC1(N)CCN(c2nsc(-c3cc(N[C@@H]4CCOC[C@H]4O)ncc3Cl)n2)CC1]

Step 3. A round bottom flask was charged with tert-butyl (1-(5-(2-(((3S,4R)-3-((tert-butyldimethylsilyl)oxy)tetrahydro-2H-pyran-4-yl)amino)-5-hydroxypyridin-4-yl)-1,2,4-thiadiazol-3-yl)-4-methylpiperidin-4-yl)carbamate (185 mg, 0.298 mmol) and HCl in 1,4-Dioxane (5 mL, 4 molar) and a stirbar, the solution was stirred for 1 hour at 25° C. The resulting mixture was concentrated under vacuum. The resulting crude material was purified by Pre-HPLC (Column: XBridge Shield RP18 OBD Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 25% B to 45% B in 7 min; Wave Length: 254 nm/220 nm nm; RT1 (min): 6.33; Number Of Runs: 3). Lyophilization yielded (3S,4R)-4-((4-(3-(4-amino-4-methylpiperidin-1-yl)-1,2,4-thiadiazol-5-yl)-5-chloropyridin-2-yl)amino) tetrahydro-2H-pyran-3-ol (28.5 mg, 22.5%) as an yellow solid. m/z (ES⁺) [M+H]⁺=425.10; HPLC tR=0.798 min. ¹H NMR (400 MHz, DMSO-d₆) δ 8.20 (s, 1H), 7.49 (s, 1H), 7.19 (d, J=7.5 Hz, 1H), 4.98 (d, J=5.2 Hz, 1H), 3.81 (dt, J=15.5, 8.3 Hz, 5H), 3.66 (ddd, J=12.9, 8.8, 4.0 Hz, 2H), 3.39 (s, 2H), 3.08 (dd, J=11.1, 9.3 Hz, 1H), 2.05-1.96 (m, 1H), 1.76 (s, 1H), 1.54-1.33 (m, 5H), 1.10 (s, 3H).

### Example 60

### (4S,5R)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol (I-982), &

### (4R,5S)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol (I-984)

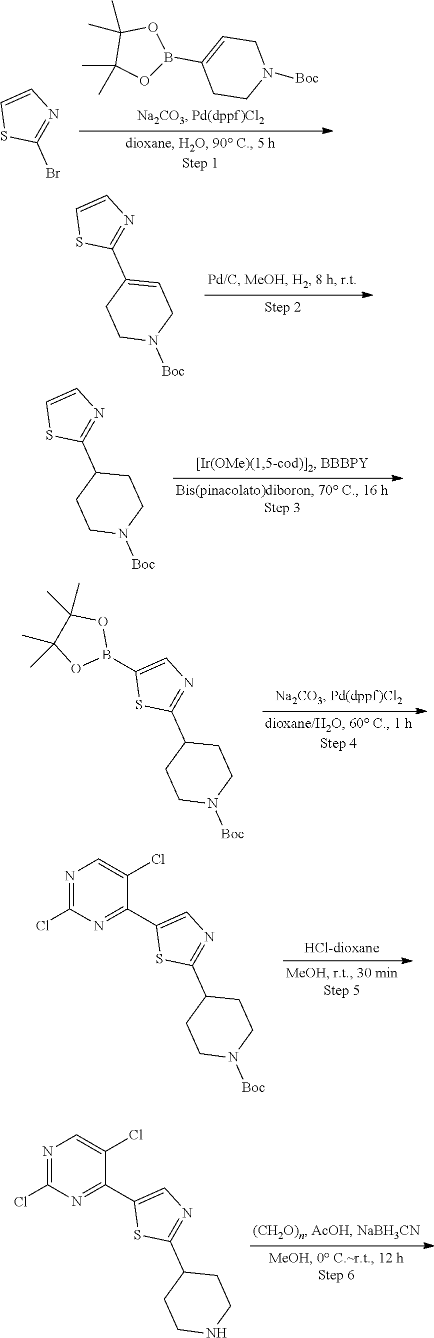

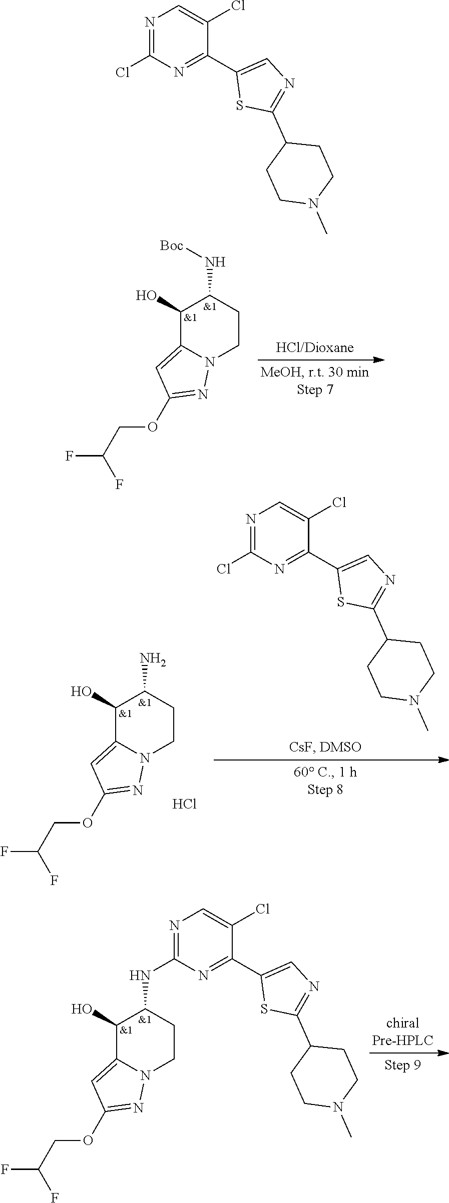 [SMILES: CN1CCC(c2ncc(-c3nc(Cl)ncc3Cl)s2)CC1 ; CC(C)(C)OC(=O)N[C@@H]1CCn2nc(OCC(F)F)cc2[C@H]1O ; N[C@@H]1CCn2nc(OCC(F)F)cc2[C@H]1O ; Cl ; CN1CCC(c2ncc(-c3nc(Cl)ncc3Cl)s2)CC1 ; CN1CCC(c2ncc(-c3nc(N[C@@H]4CCn5nc(OCC(F)F)cc5[C@H]4O)ncc3Cl)s2)CC1]

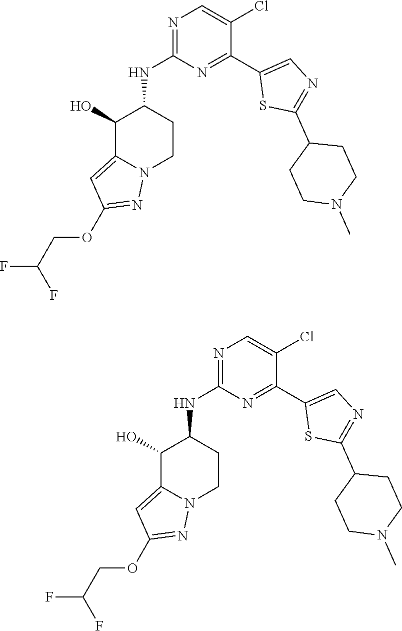 [SMILES: CN1CCC(c2ncc(-c3nc(N[C@@H]4CCn5nc(OCC(F)F)cc5[C@H]4O)ncc3Cl)s2)CC1 ; CN1CCC(c2ncc(-c3nc(N[C@H]4CCn5nc(OCC(F)F)cc5[C@@H]4O)ncc3Cl)s2)CC1]

### tert-butyl 4-(thiazol-2-yl)-3,6-dihydropyridine-1(2H)-carboxylate

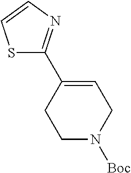 [SMILES: CC(C)(C)OC(=O)N1CC=C(c2nccs2)CC1]

Step 1. A round bottom flask was charged with tert-butyl 4-(4,4,5,5-tetramethyl-1,3,2-dioxaborolan-2-yl)-3,6-dihydropyridine-1(2H)-carboxylate (21 g, 67 mmol), 2-bromothiazole (10 g, 61 mmol), PdCl₂(dppf) (4.5 g, 6.1 mmol), Na₂CO₃ (19 g, 180 mmol), 1,4-Dioxane (160 mL) and H₂O (40 mL) and a stirbar before being evacuated and purged with nitrogen three times, and the mixture was stirred for 5 hours at 90° C. The reaction mixture was diluted with water (200 mL), and the aqueous phase was extracted with EA (100 mL) three times. The combined organic layers were washed with brine, dried over sodium sulfate, filtered, and concentrated in vacuo. The residue was purified by silica gel chromatography eluting with PE/EA; ratio: 4/1. Concentration in vacuo resulted in tert-butyl 4-(thiazol-2-yl)-3,6-dihydropyridine-1(2H)-carboxylate (7.5 g, 46%) as a yellow oil. m/z (ES⁺) [M+H]⁺=267.05; HPLC tR=0.948 min.

### tert-butyl 4-(thiazol-2-yl)piperidine-1-carboxylate

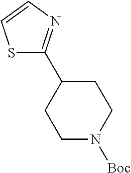 [SMILES: CC(C)(C)OC(=O)N1CCC(c2nccs2)CC1]

Step 2. A stirred mixture of tert-butyl 4-(thiazol-2-yl)-3,6-dihydropyridine-1(2H)-carboxylate (7.5 g, 28 mmol) and Pd/C (0.60 g, 5.6 mmol) in MeOH (75 mL) was treated with H₂ for 8 hours at r.t. The reaction mixture was filtered (through a pad of Celite), the pad was washed with MeOH, and the filtrate was concentrated in vacuo to get tert-butyl 4-(thiazol-2-yl)piperidine-1-carboxylate (6.7 g, 89%) as a yellow oil. m/z (ES⁺) [M+H]⁺=269.10; HPLC tR=0.705 min.

### tert-butyl 4-(5-(4,4,5,5-tetramethyl-1,3,2-dioxaborolan-2-yl)thiazol-2-yl)piperidine-1-carboxylate

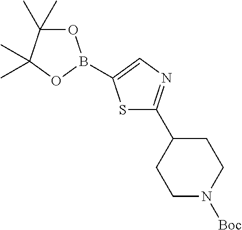 [SMILES: CC(C)(C)OC(=O)N1CCC(c2ncc(B3OC(C)(C)C(C)(C)O3)s2)CC1]

Step 3. A resealable reaction vial was charged with Bis(pinacolato)diborane (3 g, 10 mmol), tert-butyl 4-(thiazol-2-yl)piperidine-1-carboxylate (3 g, 10 mmol), 4,4′-Di-tert-butyl-2,2′-dipyridyl (0.6 g, 2 mmol), Dimethyliridium(1+)methanolate-(1Z,5Z)-1,5-cyclooctadiene (0.4 g, 0.6 mmol), THF (50 mL) and a stirbar before being evacuated and purged with nitrogen three times, and the mixture was stirred for 16 hours at 70° C. The solvent was removed by distillation under vacuum to get the crude tert-butyl 4-(5-(4,4,5,5-tetramethyl-1,3,2-dioxaborolan-2-yl)thiazol-2-yl)piperidine-1-carboxylate (3 g, 70%) as a black oil. m/z (ES⁺) [M+H]⁺=395.20; HPLC tR=1.023 min.

### tert-butyl 4-(5-(2,5-dichloropyrimidin-4-yl)thiazol-2-yl)piperidine-1-carboxylate

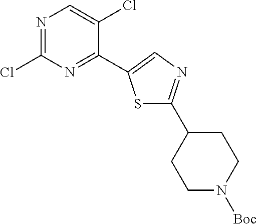 [SMILES: CC(C)(C)OC(=O)N1CCC(c2ncc(-c3nc(Cl)ncc3Cl)s2)CC1]

Step 4. A resealable reaction vial was charged with tert-butyl 4-(5-(4,4,5,5-tetramethyl-1,3,2-dioxaborolan-2-yl)thiazol-2-yl)piperidine-1-carboxylate (3 g, 8 mmol), 2,4,5-trichloropyrimidine (2 g, 10 mmol), PdCl₂(dppf) (0.6 g, 0.8 mmol), Na₂CO₃ (2 g, 20 mmol), 1,4-Dioxane (30 mL) and H₂O (7.5 mL) and a stirbar before being evacuated and purged with nitrogen three times, and the mixture was stirred for 1 hours at 60° C. The reaction mixture was diluted with water (200 mL), and the aqueous phase was extracted with EA (80 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 0% to 100% gradient in 30 min; detector, UV 254 nm. Concentration in vacuo resulted in tert-butyl 4-(5-(2,5-dichloropyrimidin-4-yl)thiazol-2-yl)piperidine-1-carboxylate (1.3 g, 40%) as a yellow oil. m/z (ES⁺) [M+H]⁺=415.10; HPLC tR=1.150 min.

### 5-(2,5-dichloropyrimidin-4-yl)-2-(piperidin-4-yl)thiazole

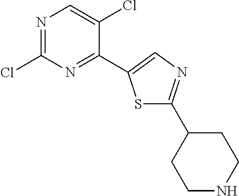 [SMILES: Clc1ncc(Cl)c(-c2cnc(C3CCNCC3)s2)n1]

Step 5. To a mixture of tert-butyl 4-(5-(2,5-dichloropyrimidin-4-yl)thiazol-2-yl)piperidine-1-carboxylate (1.28 g, 3.08 mmol) in MeOH (23 mL) was added hydrogen chloride in dioxane (23.1 mL, 55.7 mmol) dropwise at 25° C. under nitrogen atmosphere. The mixture was stirred for 30 min at 25° C. The solution was concentrated and the pH value of the residue was adjusted to 6-7 with aq.NaHCO₃. The solvent was evaporated in vacuo to give 5-(2,5-dichloropyrimidin-4-yl)-2-(piperidin-4-yl)thiazole (1.4 g, 86%, crude) as a white solid. m/z (ES⁺) [M+H]⁺=314.90; HPLC tR=0.581 min.

### 5-(2,5-dichloropyrimidin-4-yl)-2-(1-methylpiperidin-4-yl)thiazole

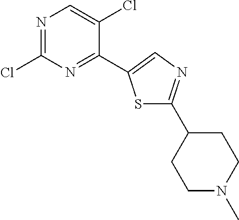 [SMILES: CN1CCC(c2ncc(-c3nc(Cl)ncc3Cl)s2)CC1]

Step 6. To a mixture of 5-(2,5-dichloropyrimidin-4-yl)-2-(piperidin-4-yl)thiazole (1.3 g, 4.1 mmol) and Paraformaldehyde (0.37 g, 0.37 mL, 12 mmol) in MeOH (10 mL) was added AcOH (0.74 g, 0.71 mL, 12 mmol) dropwise at 25° C. under air atmosphere. The mixture was stirred for 1 hour at 0° C. prior addition of NaCNBH₃ (0.78 g, 12 mmol). The mixture was stirred for 10 hours at 25° C. The reaction mixture was purified by silica gel chromatography (eluting with DCM/MeOH, ratio: 10/1). Concentration in vacuo resulted in 5-(2,5-dichloropyrimidin-4-yl)-2-(1-methylpiperidin-4-yl)thiazole (1.1 g, 81%) as a yellow solid. m/z (ES⁺) [M+H]⁺=329.00; HPLC tR=0.838 min.

### rac-(4R,5S)-5-amino-2-(2,2-difluoroethoxy)-4,5,6,7-tetrahydropyrazolo[1,5-a]pyridin-4-ol hydrochloride

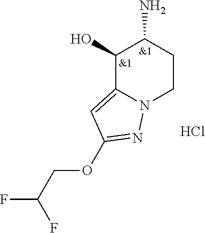 [SMILES: N[C@@H]1CCn2nc(OCC(F)F)cc2[C@H]1O ; Cl]

Step 7. To a mixture of rac-tert-butyl ((4R,5S)-2-(2,2-difluoroethoxy)-4-hydroxy-4,5,6,7-tetrahydropyrazolo[1,5-a]pyridin-5-yl)carbamate (300 mg, 0.90 mmol) in MeOH (0.5 mL) was added hydrogen chloride in dioxane (1.5 mL, 37.5 mmol) dropwise at 25° C. under nitrogen atmosphere. The mixture was stirred for 30 min at 25° C. The solution was concentrated. This resulted in rac-(4R,5S)-5-amino-2-(2,2-difluoroethoxy)-4,5,6,7-tetrahydropyrazolo[1,5-a]pyridin-4-ol hydrochloride (212 mg, 78%, crude) as a white solid. m/z (ES⁺) [M+H]⁺=234.00; HPLC tR=0.358 min.

### rac-(4R,5S)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol

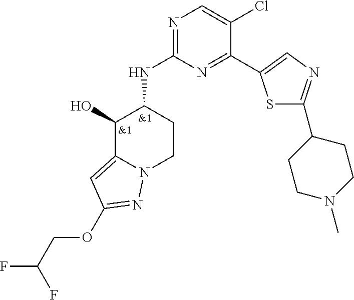 [SMILES: CN1CCC(c2ncc(-c3nc(N[C@@H]4CCn5nc(OCC(F)F)cc5[C@H]4O)ncc3Cl)s2)CC1]

Step 8. A resealable reaction vial was charged with rac-(4R,5S)-5-amino-2-(2,2-difluoroethoxy)-4,5,6,7-tetrahydropyrazolo[1,5-a]pyridin-4-ol hydrochloride (151 mg, 0.56 mmol), 5-(2,5-dichloropyrimidin-4-yl)-2-(1-methylpiperidin-4-yl)thiazole (184 mg, 0.56 mmol), CsF (425 mg, 2.80 mmol), DMSO (2 mL) and the mixture was stirred for 1 hour at 60° C. The reaction mixture was diluted with water (20 mL), and the aqueous phase was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 50% to 100% gradient in 20 min; detector, UV 254 nm. Concentration in vacuo resulted in rac-(4R,5S)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol (40 mg, 13%) as a white solid. m/z (ES⁺) [M+H]⁺=526.15; HPLC tR=0.728 min.

### (4S,5R)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol &

### (4R,5S)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol

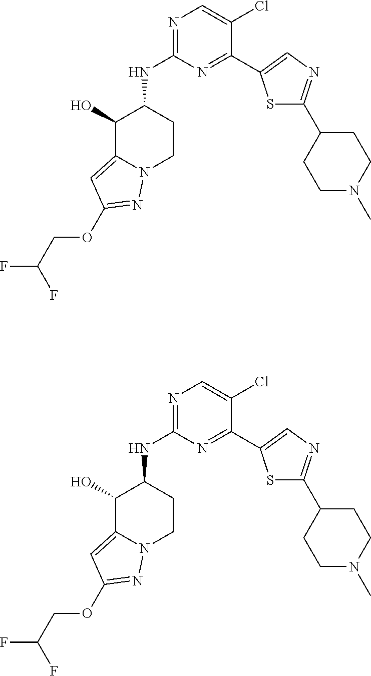 [SMILES: CN1CCC(c2ncc(-c3nc(N[C@@H]4CCn5nc(OCC(F)F)cc5[C@H]4O)ncc3Cl)s2)CC1 ; CN1CCC(c2ncc(-c3nc(N[C@H]4CCn5nc(OCC(F)F)cc5[C@@H]4O)ncc3Cl)s2)CC1]

Step 9. rac-(4R,5S)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol (25 mg, 0.05 mmol) was purified by chiral Pre-HPLC (Column: CHIRALPAK ID, 2*25 cm, 5 μm; Mobile Phase A: Hex(0.5% 2M NH₃-MeOH), Mobile Phase B: EtOH:DCM=1:1-HPLC; Flow rate: 20 mL/min; Gradient: 40% B to 40% B in 20 min; Wave Length: 220/254 nm; RT1 (min): 11.02; RT2 (min): 15.23; Sample Solvent: EtOH:DCM=1:1-HPLC; Injection Volume: 1.4 mL; Number Of Runs: 3).

Lyophilization of eluted peak 1 yielded (4S,5R)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol (5.8 mg, 23%,) as a white solid. m/z (ES⁺) [M+H]⁺=526.10; HPLC tR=0.898 min. ¹H NMR (400 MHz, DMSO-d₆) δ 8.68 (s, 1H), 8.47 (s, 1H), 7.65 (s, 1H), 6.34 (s, 1H), 5.78-5.64 (m, 2H), 4.68 (s, 1H), 4.33 (td, J=15.0, 3.5 Hz, 2H), 4.07 (s, 3H), 2.99 (d, J=11.8 Hz, 1H), 2.86 (d, J=11.2 Hz, 2H), 2.22 (s, 4H), 2.12-1.91 (m, 5H), 1.75 (d, J=12.3 Hz, 2H).

Lyophilization of eluted peak 2 yielded (4R,5S)-5-((5-chloro-4-(2-(1-methylpiperidin-4-yl)thiazol-5-yl)pyrimidin-2-yl)amino)-2-(2,2-difluoroethoxy)-4,5,6,7-tetrahydropyrazolo[1,5-a]pyridin-4-ol (7.5 mg, 29%,) as a white solid. m/z (ES⁺) [M+H]⁺=526.10; HPLC tR=1.325 min. ¹H NMR (400 MHz, DMSO-d₆) δ 8.68 (s, 1H), 8.47 (s, 1H), 7.66 (s, 1H), 6.51-6.18 (m, 1H), 5.75-5.68 (m, 2H), 4.68 (s, 1H), 4.33 (td, J=14.9, 3.6 Hz, 2H), 4.17-3.88 (m, 3H), 3.03 (s, 1H), 2.89 (s, 2H), 2.29 (d, J=32.1 Hz, 4H), 2.02 (dd, J=30.6, 11.4 Hz, 5H), 1.76 (d, J=12.7 Hz, 2H).

### Example 61

### (3S,4R)-4-((9-(1-methylpiperidin-4-yl)-6,7-dihydrothiazolo[5′,4′:4,5]oxepino[3,2-d]pyrimidin-2-yl)amino)tetrahydro-2H-pyran-3-ol (I-1073)

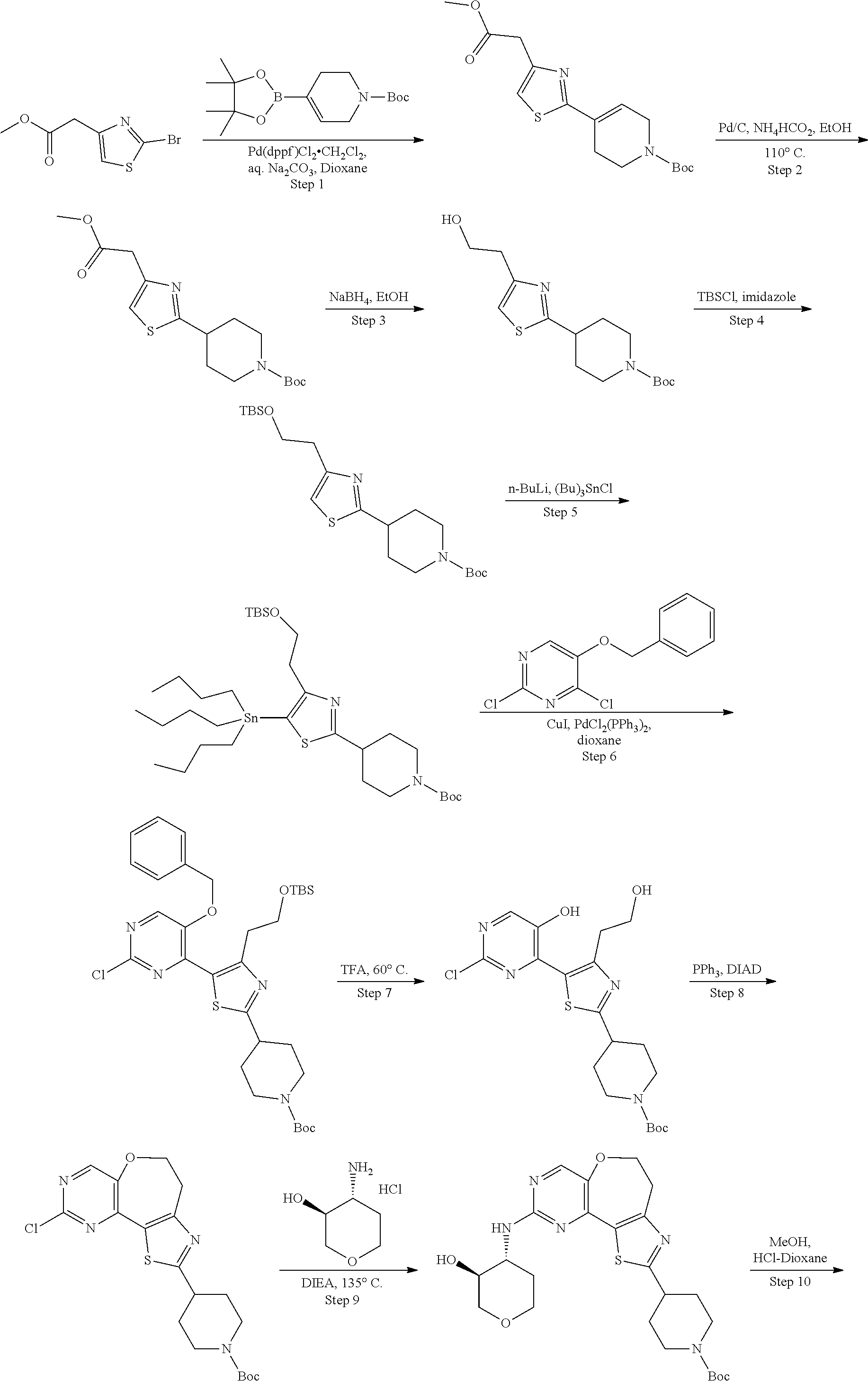

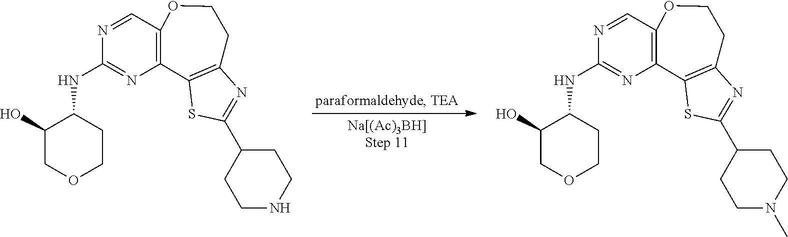 [SMILES: O[C@@H]1COCC[C@H]1Nc1ncc2c(n1)-c1sc(C3CCNCC3)nc1CCO2 ; CN1CCC(c2nc3c(s2)-c2nc(N[C@@H]4CCOC[C@H]4O)ncc2OCC3)CC1]

### tert-butyl 4-(4-(2-methoxy-2-oxoethyl)thiazol-2-yl)-3,6-dihydropyridine-1(2H)-carboxylate

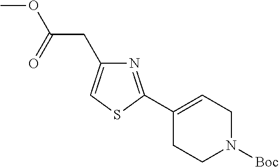 [SMILES: COC(=O)Cc1csc(C2=CCN(C(=O)OC(C)(C)C)CC2)n1]

Step 1. To a mixture of sodium carbonate (2.020 g, 3.000 Eq, 19.06 mmol) in Water (5.0 mL) and 1,4-dioxane (20 mL) in a sealable vessel, with nitrogen being bubbled in, were added sequentially tert-butyl 4-(4,4,5,5-tetramethyl-1,3,2-dioxaborolan-2-yl)-3,6-dihydropyridine-1 (2H)-carboxylate (2.947 g, 1.500 Eq, 9.531 mmol), methyl 2-(2-bromothiazol-4-yl)acetate (1.500 g, 1.000 Eq, 6.354 mmol), and [1,1′-Bis(diphenylphosphino)ferrocene]dichloropalladium (II) complex with dichloromethane (518.9 mg, 0.100 Eq, 635.4 μmol). The vessel was sealed, and the mixture was stirred at 90° C. for 90 min. The reaction mixture was cooled to R.T. The solvents were decanted (the cake was washed several times with EtOAc) and dried over sodium sulfate, filtered, and concentrated down. The dark crude oil was purified by silica gel chromatography a heptane to EtOAc gradient to yield tert-butyl 4-(4-(2-methoxy-2-oxoethyl)thiazol-2-yl)-3,6-dihydropyridine-1(2H)-carboxylate (2.0988 g, 6.2018 mmol, 97.61%) as a yellow oil. LCMS RT=1.46 min., ESI MS (M+H) m/z=339.11.

### tert-butyl 4-(4-(2-methoxy-2-oxoethyl)thiazol-2-yl)piperidine-1-carboxylate

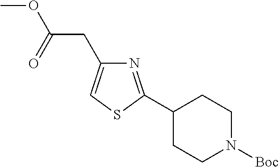 [SMILES: COC(=O)Cc1csc(C2CCN(C(=O)OC(C)(C)C)CC2)n1]

Step 2. A mixture of tert-butyl 4-(4-(2-methoxy-2-oxoethyl)thiazol-2-yl)-3,6-dihydropyridine-1(2H)-carboxylate (2.0988 g, 1.000 Eq, 6.2018 mmol), ammonium formate (7.822 g, 6.173 mL, 20.00 Eq, 124.04 mmol), and palladium on carbon (10 wt %, wet) (2.6400 g, 10% Wt, 0.400 Eq, 2.4807 mmol) in Ethanol (70 mL) was stirred at 110° C. for 150 min in a round bottom flask open to air and fitted with a condenser. The mixture was cooled to R.T. and filtered through a celite pad with EtOAc. The filtrate was concentrated down and dissolved into EtOAc (~120 mL) and water (~60 mL). The layers were separated, and the aqueous layer was extracted with fresh EtOAc (50 mL). The organic layers were washed once with brine (60 mL), combined, dried over sodium sulfate, filtered, concentrated down, and dried to yield tert-butyl 4-(4-(2-methoxy-2-oxoethyl)thiazol-2-yl)piperidine-1-carboxylate (1.637 g, 4.7 mmol, 75%, 97% Purity) as a light yellow oil. LCMS RT=1.42 min., ESI MS (M-56) m/z=285.11.

### tert-butyl 4-(4-(2-hydroxyethyl)thiazol-2-yl)piperidine-1-carboxylate

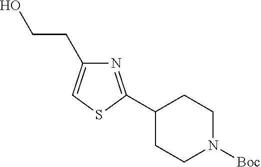 [SMILES: CC(C)(C)OC(=O)N1CCC(c2nc(CCO)cs2)CC1]

Step 3. A mixture of tert-butyl 4-(4-(2-methoxy-2-oxoethyl)thiazol-2-yl)piperidine-1-carboxylate (1.637 g, 97% Wt, 1.000 Eq, 4.664 mmol) and sodium borohydride (352.9 mg, 2.000 Eq, 9.328 mmol) in ethanol (20 mL) was stirred for 72 hours at 80° C. Additional sodium borohydride was added 4 times during the reaction for an additional total of 3.53 g, 10.0 Eq, 93.3 mmol. The reaction mixture was then cooled to R.T., treated with saturated aqueous ammonium chloride (100 mL), water (100 mL), and ethyl acetate (200 mL). The layers were separated, and the aqueous layer was extracted with fresh ethyl acetate (200 mL). The combined organic layers were brine-washed, dried over sodium sulfate, filtered, and concentrated down. The dark gray crude solid was purified by silica gel chromatography with DCM to EtOAc gradient to yield tert-butyl 4-(4-(2-hydroxyethyl)thiazol-2-yl)piperidine-1-carboxylate (1.034 g, 3.310 mmol, 70.96%) as a clear oil, which became a white solid upon standing. LCMS RT=1.24 min., ESI MS (M+1) m/z=313.16.

### tert-butyl 4-(4-(2-((tert-butyldimethylsilyl)oxy)ethyl)thiazol-2-yl)piperidine-1-carboxylate

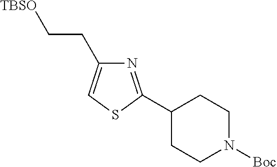 [SMILES: CC(C)(C)OC(=O)N1CCC(c2nc(CCO[Si](C)(C)C(C)(C)C)cs2)CC1]

Step 4. To a 0° C. stirred solution of tert-butyl 4-(4-(2-hydroxyethyl)thiazol-2-yl)piperidine-1-carboxylate (1.034 g, 1.000 Eq, 3.310 mmol) in DCM (10 mL) was added imidazole (277.1 mg, 1.230 Eq, 4.071 mmol) followed by TBS-C₁ (613.5 mg, 1.230 Eq, 4.071 mmol). The mixture was warmed to R.T. After about 30 minutes, the reaction was diluted with DCM (10 mL) and water (10 mL). The layers were separated, and the aqueous layer was extracted with fresh DCM (20 mL). The combined organic layers were dried over sodium sulfate, filtered, concentrated down, and dried. The clear crude oil was purified by silica gel chromatography with a heptane to 50% EtOAc/heptane gradient over 22 minutes. The major product peak eluted between 15% and 27% EtOAc/heptane to yield tert-butyl 4-(4-(2-((tert-butyldimethylsilyl)oxy)ethyl)thiazol-2-yl)piperidine-1-carboxylate (1.399 g, 3.279 mmol, 99.07%) as a clear oil. LCMS RT=2.01 min., ESI MS (M+1) m/z=427.32.

### tert-butyl 4-(4-(2-((tert-butyldimethylsilyl)oxy)ethyl)-5-(tributylstannyl)thiazol-2-yl)piperidine-1-carboxylate

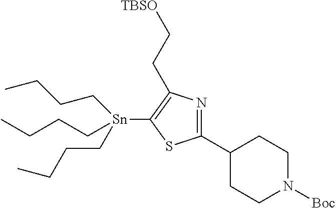 [SMILES: CCC[CH2][Sn]([CH2]CCC)([CH2]CCC)[c]1sc(C2CCN(C(=O)OC(C)(C)C)CC2)nc1CCO[Si](C)(C)C(C)(C)C]

Step 5. To a stirred −65° C. solution of tert-butyl 4-(4-(2-((tert-butyldimethylsilyl)oxy)ethyl)thiazol-2-yl)piperidine-1-carboxylate (1.399 g, 1.000 Eq, 3.279 mmol) in THF (25 mL) under nitrogen was added n-butyllithium (2.5 N in hexanes) (283.5 mg, 1.771 mL, 2.5 molar, 1.350 Eq, 4.426 mmol). The reaction was stirred for 1 hour at −65° C. before dropwise addition over 15 minutes of tributyltin chloride (1.174 g, 0.98 mL, 1.100 Eq, 3.607 mmol). After addition, the reaction was stirred for 1 hour at −65° C. The reaction was monitored by TLC (1:5 EtOAc/Heptane): the product had an Rf=~0.4. TLC indicated >70% consumption of starting material. The product was too non-polar for LC-MS detection. The reaction was quenched with water (6-7 mL) and warmed to R.T., then diluted with EtOAc (60 mL) and water (20 mL). The layers were separated, and the organic layer was brine-washed (20 mL), dried over sodium sulfate, filtered, and concentrated down. The crude thick, yellow oil was purified by silica gel chromatography with a heptane to 30% EtOAc/heptane gradient to yield tert-butyl 4-(4-(2-((tert-butyldimethylsilyl)oxy)ethyl)-5-(tributylstannyl)thiazol-2-yl)piperidine-1-carboxylate (1.149 g, 1.605 mmol, 48.96%) as a light yellow oil.

### tert-butyl 4-(5-(5-(benzyloxy)-2-chloropyrimidin-4-yl)-4-(2-((tert-butyldimethylsilyl)oxy)ethyl)thiazol-2-yl)piperidine-1-carboxylate

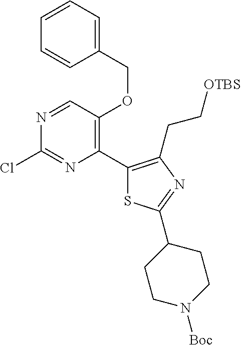 [SMILES: CC(C)(C)OC(=O)N1CCC(c2nc(CCO[Si](C)(C)C(C)(C)C)c(-c3nc(Cl)ncc3OCc3ccccc3)s2)CC1]

Step 6. A mixture of 5-(benzyloxy)-2,4-dichloropyrimidine (778.1 mg, 1.900 Eq, 3.050 mmol), tert-butyl 4-(4-(2-((tert-butyldimethylsilyl)oxy)ethyl)-5-(tributylstannyl)thiazol-2-yl)piperidine-1-carboxylate (1.149 g, 1.000 Eq, 1.605 mmol), copper(I) iodide (24.46 mg, 0.08 Eq, 128.4 mol), and Bis-(triphenylphosphino)-palladous chloride (90.14 mg, 0.08 Eq, 128.4 mol) in 1,4-Dioxane (11.0 mL) was degassed with nitrogen in a sealable vessel. The vessel was then sealed and stirred at 110° C. for 120 min. The reaction was cooled to R.T. and quenched with a solution of KF in water (0.6 g in 20 mL). After stirring for 5 minutes, the mixture was diluted with water (20 mL). A sticky dark green solid was isolated by decanting and filtration, dried under vacuum, mostly dissolved into 5% MeOH/DCM (140 mL), filtered, and concentrated down. This crude was treated with DCM (~10 mL). The solids were removed by filtration and washed with DCM (3×5 mL). The combined filtrate was concentrated down to yield a light yellow crude solid (~1.6 g). This crude was purified by silica gel chromatography with a heptane to 60% EtOAc/heptane gradient to yield tert-butyl 4-(5-(5-(benzyloxy)-2-chloropyrimidin-4-yl)-4-(2-((tert-butyldimethylsilyl)oxy)ethyl)thiazol-2-yl)piperidine-1-carboxylate (705.4 mg, 1.0 mmol, 65%, 95% Purity) as an off-white solid. LCMS RT=2.16 min., ESI MS (M+1) m/z=646.03.

### tert-butyl 4-(5-(2-chloro-5-hydroxypyrimidin-4-yl)-4-(2-hydroxyethyl)thiazol-2-yl)piperidine-1-carboxylate

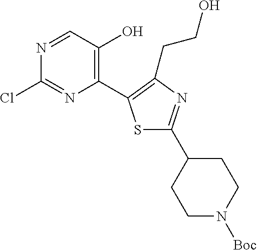 [SMILES: CC(C)(C)OC(=O)N1CCC(c2nc(CCO)c(-c3nc(Cl)ncc3O)s2)CC1]

Step 7. In a sealed tube, a mixture of tert-butyl 4-(5-(5-(benzyloxy)-2-chloropyrimidin-4-yl)-4-(2-((tert-butyldimethylsilyl)oxy)ethyl)thiazol-2-yl)piperidine-1-carboxylate (705.4 mg, 95% Wt, 1.000 Eq, 1.038 mmol) in TFA (1.776 g, 1.200 mL, 15.000 Eq, 15.58 mmol) was stirred at 60° C. for 65 hours, then at 90° C. for 30 minutes. The mixture was concentrated down, then dissolved into MeOH (5 mL), and treated with HCl (4N in Dioxane) (567.9 mg, 3.894 mL, 4 molar, 15.000 Eq, 15.58 mmol). This mixture was stirred at room temperature for 40 minutes, then concentrated down, and dried. To the crude residue were added DCM (10 mL), TEA (2.627 g, 3.62 mL, 25.00 Eq, 25.96 mmol), and Boc anhydride (226.6 mg, 239 μL, 1.000 Eq, 1.038 mmol). This mixture was stirred for 15 minutes, then concentrated down. The crude was purified by silica gel chromatography with isocratic EtOAc followed by a quick EtOAc to 20% MeOH/EtOAc gradient, then isocratic 20% MeOH/EtOAc to yield tert-butyl 4-(5-(2-chloro-5-hydroxypyrimidin-4-yl)-4-(2-hydroxyethyl)thiazol-2-yl)piperidine-1-carboxylate (149.3 mg, 0.32 mmol, 31%, 95% Purity) as dark yellow, foamy solid. LCMS RT=1.34 min., ESI MS (M+1) m/z=441.17.

### tert-butyl 4-(2-chloro-6,7-dihydrothiazolo[5′,4′:4,5]oxepino[3,2-d]pyrimidin-9-yl)piperidine-1-carboxylate

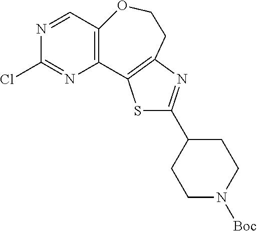 [SMILES: CC(C)(C)OC(=O)N1CCC(c2nc3c(s2)-c2nc(Cl)ncc2OCC3)CC1]

Step 8. To a 0° C. stirred solution of tert-butyl 4-(5-(2-chloro-5-hydroxypyrimidin-4-yl)-4-(2-hydroxyethyl)thiazol-2-yl)piperidine-1-carboxylate (149.3 mg, 95% Wt, 1.000 Eq, 321.7 μmol) and triphenylphosphine (109.7 mg, 92.64 μL, 1.300 Eq, 418.2 μmol) in THF (10 mL) was added diisopropyl diazene-1,2-dicarboxylate (84.56 mg, 82.49 μL, 1.300 Eq, 418.2 μmol) over 5 minutes. The water bath was removed, and the reaction mixture was warmed to room temperature while stirring for 40 minutes. The solvents were removed under reduced pressure, and the crude was purified by silica gel chromatography with a heptane to EtOAc gradient to yield tert-butyl 4-(2-chloro-6,7-dihydrothiazolo[5′,4′:4,5]oxepino[3,2-d]pyrimidin-9-yl)piperidine-1-carboxylate (41.5 mg, 93 μmol, 29%, 95% Purity) as an off-white foamy solid. LCMS RT=1.64 min., ESI MS (M-56) m/z=367.06.

### tert-butyl 4-(2-chloro-6,7-dihydrothiazolo[5′,4′:4,5]oxepino[3,2-d]pyrimidin-9-yl)piperidine-1-carboxylate

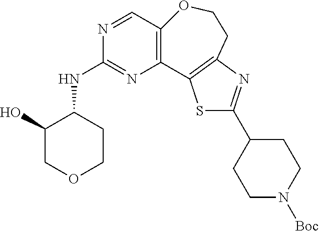 [SMILES: CC(C)(C)OC(=O)N1CCC(c2nc3c(s2)-c2nc(N[C@@H]4CCOC[C@H]4O)ncc2OCC3)CC1]

Step 9. In a sealed tube, a mixture of tert-butyl 4-(2-chloro-6,7-dihydrothiazolo[5′,4′:4,5]oxepino[3,2-d]pyrimidin-9-yl)piperidine-1-carboxylate (41.5 mg, 95% Wt, 1.000 Eq, 93.2 μmol), (3S,4R)-4-Amino-tetrahydropyran-3-ol hydrochloride (25.8 mg, 1.800 Eq, 168 mol), and hunig'sbase (60.2 mg, 81.2 μL, 5.000 Eq, 466 mol) in DMSO (1 mL) was stirred at 135° C. for 20 hours, then at 145° C. for 5 hours. The reaction mixture was cooled to room temperature, treated with water (15 mL), and extracted with EtOAc (2×30 mL). The combined organic layers were washed with water (2×10 mL) and then with brine (2×10 mL), dried over sodium sulfate, filtered, concentrated down, and dried. The crude was purified by silica gel chromatography with a DCM to EtOAc gradient followed by isocratic EtOAc. The partially pure product was then purified by silica gel thin layer preparative chromatography with 5% MeOH/EtOAc to yield tert-butyl 4-(2-(((3S,4R)-3-hydroxytetrahydro-2H-pyran-4-yl)amino)-6,7-dihydrothiazolo[5′,4′:4,5]oxepino[3,2-d]pyrimidin-9-yl)piperidine-1-carboxylate (2.1 mg, 4.0 μmol, 4.2%, 95% Purity) as an amber film. LCMS RT=1.34 min., ESI MS (M+1) m/z=504.22.

### (3S,4R)-4-((9-(piperidin-4-yl)-6,7-dihydrothiazolo[5′,4′:4,5]oxepino[3,2-d]pyrimidin-2-yl)amino)tetrahydro-2H-pyran-3-ol

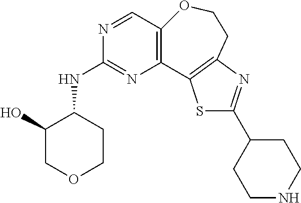 [SMILES: O[C@@H]1COCC[C@H]1Nc1ncc2c(n1)-c1sc(C3CCNCC3)nc1CCO2]

Step 10. A mixture of tert-butyl 4-(2-(((3S,4R)-3-hydroxytetrahydro-2H-pyran-4-yl)amino)-6,7-dihydrothiazolo[5′,4′:4,5]oxepino[3,2-d]pyrimidin-9-yl)piperidine-1-carboxylate (2.1 mg, 95% Wt, 1.000 Eq, 4.0 mol) in MeOH (0.3 mL) and hydrogen chloride (4N in dioxane) (72 mg, 0.50 mL, 4 molar, 500 Eq, 2.0 mmol) was stirred vigorously for 15 min at 23° C. The light amber solution was concentrated down and dried under high vacuum to yield crude (3S,4R)-4-((9-(piperidin-4-yl)-6,7-dihydrothiazolo[5′,4′:4,5]oxepino[3,2-d]pyrimidin-2-yl)amino)tetrahydro-2H-pyran-3-ol, HCl as a thick, light amber oil, which was used immediately for the next step. LCMS RT=0.82 min., ESIMS (M+1) m/z=404.16.

### (3S,4R)-4-((9-(1-methylpiperidin-4-yl)-6,7-dihydrothiazolo[5′,4′:4,5]oxepino[3,2-d]pyrimidin-2-yl)amino)tetrahydro-2H-pyran-3-ol

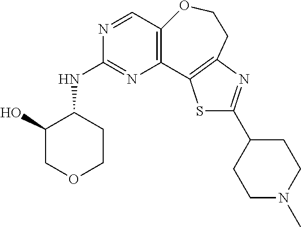 [SMILES: CN1CCC(c2nc3c(s2)-c2nc(N[C@@H]4CCOC[C@H]4O)ncc2OCC3)CC1]

Step 11. To a stirred solution of (3S,4R)-4-((9-(piperidin-4-yl)-6,7-dihydrothiazolo[5′,4′:4,5]oxepino[3,2-d]pyrimidin-2-yl)amino)tetrahydro-2H-pyran-3-ol, HCl (1.8 mg, 1.000 Eq, 4.0 mol) and triethylamine (2.0 mg, 2.8 μL, 5.000 Eq, 20 mol) in DCM (0.5 mL) and MeOH (0.05 mL) was added Paraformaldehyde (2.4 mg, 2.3 μL, 20.0 Eq, 80 mol). The mixture was stirred 30 minutes before addition of sodium triacetoxyborohydride (4.2 mg, 5.00 Eq, 20 mol). The reaction was stirred for 30 minutes after the addition of reducing agent. The reaction was quenched with aqueous 2N NaOH (~0.7 mL), stirred for 10 minutes, and then extracted with DCM (4×2 mL). The combined organic extracts were dried over sodium sulfate, filtered, concentrated down, and purified by silica gel thin layer preparative chromatography with 10:90:0.5 MeOH/DCM/NH₄OH to yield (3S,4R)-4-((9-(1-methylpiperidin-4-yl)-6,7-dihydrothiazolo[5′,4′:4,5]oxepino[3,2-d]pyrimidin-2-yl)amino)tetrahydro-2H-pyran-3-ol (1.8 mg, 4.1 μmol, 100%, 95% Purity). LCMS RT=0.81 min., ESI MS (M+1) m/z=418.22.

### Example 62

### (3S,4R)-4-((2-(1-methylpiperidin-4-yl)-4,5-dihydrothiazolo[4,5-h]quinazolin-8-yl)amino)tetrahydro-2H-pyran-3-ol (I-1050)

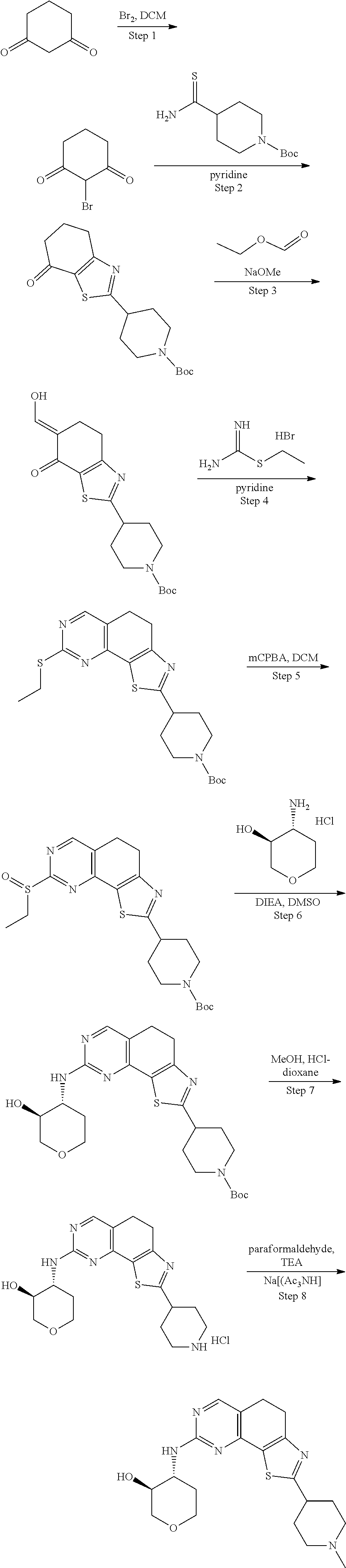

### 2-bromocyclohexane-1,3-dione

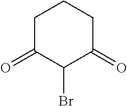

Step 1. To a stirred 0° C. solution of cyclohexane-1,3-dione (10.04 g, 1.000 Eq, 89.54 mmol) in DCM (22 mL) was added dropwise over 10-15 minutes a solution of bromine (11.13 g, 3.589 mL, 0.778 Eq, 69.66 mmol) in DCM (3.4 mL). After addition, the ice water bath was removed, and the reaction was stirred for 60 minutes. The light beige solid was isolated by filtration and washed with DCM (2×60 mL), followed by toluene (40 mL), and dried. This solid, which weighed about 20 g, was treated with water (~100 mL). An off-white solid was isolated by filtration, washed with water (3×50 mL), and dried to yield 2-bromocyclohexane-1,3-dione (13.892 g, 65 mmol, 73%, 90% Purity). LCMS RT=0.57 min., ESI MS (M+1) m/z=191.00.

### tert-butyl 4-(7-oxo-4,5,6,7-tetrahydrobenzo[d]thiazol-2-yl)piperidine-1-carboxylate

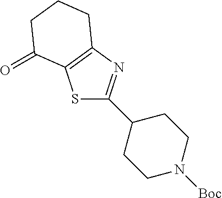 [SMILES: CC(C)(C)OC(=O)N1CCC(c2nc3c(s2)C(=O)CCC3)CC1]

Step 2. A mixture of tert-butyl 4-carbamothioylpiperidine-1-carboxylate (1.500 g, 1.000 Eq, 6.139 mmol) and 2-bromocyclohexane-1,3-dione (1.303 g, 90% Wt, 1.000 Eq, 6.139 mmol) in pyridine (11.3 mL) was stirred 18 hours at 50° C. The mixture became a yellow solution. After 18 hours, the bulk of pyridine was removed under reduced pressure. The crude was purified by silica gel chromatography requiring two columns, the first one eluted with a DCM to 70% EtOAc/DCM gradient, and the second column eluted with a heptane to EtOAc gradient, to yield tert-butyl 4-(7-oxo-4,5,6,7-tetrahydrobenzo[d]thiazol-2-yl)piperidine-1-carboxylate (1.241 g, 3.689 mmol, 60.09%) as a thick oil. LCMS RT=1.35 min., ESI MS (M-56) m/z=281.16.

### tert-butyl (E)-4-(6-(hydroxymethylene)-7-oxo-4,5,6,7-tetrahydrobenzo[d]thiazol-2-yl)piperidine-1-carboxylate

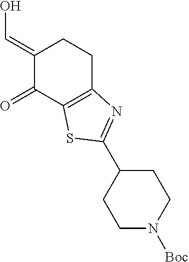 [SMILES: CC(C)(C)OC(=O)N1CCC(c2nc3c(s2)C(=O)/C(=C/O)CC3)CC1]

Step 3. A mixture of tert-butyl 4-(7-oxo-4,5,6,7-tetrahydrobenzo[d]thiazol-2-yl)piperidine-1-carboxylate (681.1 mg, 1.000 Eq, 2.024 mmol) and sodium methoxide (2.187 g, 20.0 Eq, 40.49 mmol) in THE (14 mL) was stirred at R.T. for 20 minutes before being placed over an ice water bath. Ethyl formate (2.999 g, 3.27 mL, 20.00 Eq, 40.49 mmol) was added dropwise over 5 minutes to the cooled dark brown mixture. After addition, the ice water bath was removed, and the reaction mixture was stirred for 45 minutes at 23° C. The solvent was then removed under reduced pressure. The residue was treated with saturated aqueous NH₄Cl so that pH=−6. The aqueous mixture was extracted with EtOAc (2×30 mL). The combined organic layers were dried over sodium sulfate, filtered, concentrated down, and dried to yield tert-butyl (E)-4-(6-(hydroxymethylene)-7-oxo-4,5,6,7-tetrahydrobenzo[d]thiazol-2-yl)piperidine-1-carboxylate (950.3 mg, 2.1 mmol, 100%, 80% Purity) as a tan, foamy oil. LCMS RT=1.38 min., ESI MS (M-56) m/z=309.11.

### tert-butyl 4-(8-(ethylthio)-4,5-dihydrothiazolo[4,5-h]quinazolin-2-yl)piperidine-1-carboxylate

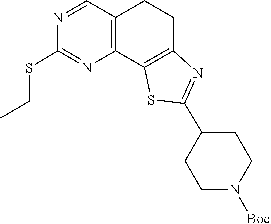 [SMILES: CCSc1ncc2c(n1)-c1sc(C3CCN(C(=O)OC(C)(C)C)CC3)nc1CC2]

Step 4. A mixture of tert-butyl (E)-4-(6-(hydroxymethylene)-7-oxo-4,5,6,7-tetrahydrobenzo[d]thiazol-2-yl)piperidine-1-carboxylate (897.0 mg, 80% Wt, 1.000 Eq, 1.969 mmol) and ethyl carbamimidothioate hydrobromide (911.0 mg, 766 μL, 2.500 Eq, 4.922 mmol) in pyridine (3.660 g, 3.74 mL, 23.5 Eq, 46.27 mmol) was stirred for 20 minutes at 23° C., and then for 28 hours at 70° C. During the 70° C. portion, additional ethyl carbamimidothioate hydrobromide was added twice, at 5 hours (600 mg) and at 23 hours (600 mg). After cooling down the reaction to R.T., the bulk of pyridine was removed by dissolving into a mix of EtOAc (~80 mL) and dilute aqueous citric acid solution (pH=~4, ~30 mL). The layers were separated, and the organic layer was washed successively with additional citric acid followed by aqueous 2N NaOH, and then brine. The organic layer was dried over sodium sulfate, filtered, concentrated down, and dried under high vacuum. The crude amber oil was purified by silica gel chromatography with a heptane to EtOAc gradient to yield tert-butyl 4-(8-(ethylthio)-4,5-dihydrothiazolo[4,5-h]quinazolin-2-yl)piperidine-1-carboxylate (176.1 mg, 407.1 mol, 20.67%) as yellow oil. LCMS RT=1.71 min., ESI MS (M+1) m/z=433.17.

### tert-butyl 4-(8-(ethylsulfinyl)-4,5-dihydrothiazolo[4,5-h]quinazolin-2-yl)piperidine-1-carboxylate

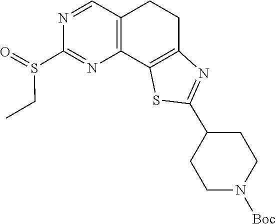 [SMILES: CCS(=O)c1ncc2c(n1)-c1sc(C3CCN(C(=O)OC(C)(C)C)CC3)nc1CC2]

Step 5. To a stirred 0° C. solution of tert-butyl 4-(8-(ethylthio)-4,5-dihydrothiazolo[4,5-h]quinazolin-2-yl)piperidine-1-carboxylate (176.1 mg, 1.000 Eq, 407.1 mol) in DCM (4 mL) was added mCPBA (100.3 mg, 77% Wt, 1.100 Eq, 447.8 mol). The reaction was stirred at 0° C. for 10 minutes, and for 40 minutes after removal of ice water bath. The reaction solution was quenched with 20% aqueous sodium thiosulfate (~3 mL) and stirred for 3 minutes, then treated with saturated aqueous NaHCO3 (4 mL) and DCM (5 mL). The layers were separated, and the aqueous layer was extracted with fresh DCM (2×5 mL). The combined organic layers were dried over sodium sulfate, filtered, concentrated down, and dried to yield tert-butyl 4-(8-(ethylsulfinyl)-4,5-dihydrothiazolo[4,5-h]quinazolin-2-yl)piperidine-1-carboxylate (181.1 mg, 403.7 mol, 99.17%) as a thick, yellow foamy oil. LCMS RT=1.29 min., ESI MS (M-56) m/z=393.11.

### tert-butyl 4-(8-(((3S,4R)-3-hydroxytetrahydro-2H-pyran-4-yl)amino)-4,5-dihydrothiazolo[4,5-h]quinazolin-2-yl)piperidine-1-carboxylate

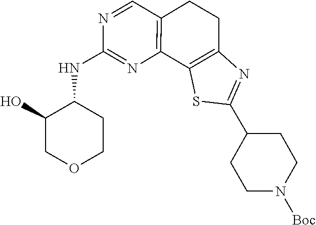 [SMILES: CC(C)(C)OC(=O)N1CCC(c2nc3c(s2)-c2nc(N[C@@H]4CCOC[C@H]4O)ncc2CC3)CC1]

Step 6. A mixture of tert-butyl 4-(8-(ethylsulfinyl)-4,5-dihydrothiazolo[4,5-h]quinazolin-2-yl)piperidine-1-carboxylate (181.1 mg, 1.000 Eq, 403.7 mol), (3S,4R)-4-aminotetrahydro-2H-pyran-3-ol HCl (111.6 mg, 1.800 Eq, 726.7 mol), and DIEA (208.7 mg, 281 μL, 4.000 Eq, 1.615 mmol) in DMSO (2 mL) was stirred 16 hour at 135° C. in a sealed tube. The reaction was cooled to R.T., treated with water (~25 mL). A light orange, sticky solid was formed. Water was removed by decanting (including 2×5 mL rinses). The light orange crude was dried under vacuum and by silica gel chromatography with a DCM to 10:90:0.5 MeOH/DCM/NH4 gradient to yield tert-butyl 4-(8-(((3S,4R)-3-hydroxytetrahydro-2H-pyran-4-yl)amino)-4,5-dihydrothiazolo[4,5-h]quinazolin-2-yl)piperidine-1-carboxylate (13.9 mg, 27 μmol, 6.7%, 95% Purity) as a light yellow, thick oil. LCMS RT=1.19 min., ESI MS (M+1) m/z=488.22.

### (3S,4R)-4-((2-(piperidin-4-yl)-4,5-dihydrothiazolo[4,5-h]quinazolin-8-yl)amino)tetrahydro-2H-pyran-3-ol hydrochloride

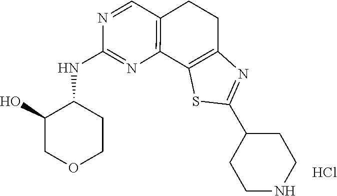 [SMILES: O[C@@H]1COCC[C@H]1Nc1ncc2c(n1)-c1sc(C3CCNCC3)nc1CC2 ; Cl]

Step 7. A mixture of tert-butyl 4-(8-(((3S,4R)-3-hydroxytetrahydro-2H-pyran-4-yl)amino)-4,5-dihydrothiazolo[4,5-h]quinazolin-2-yl)piperidine-1-carboxylate (13.9 mg, 95% wt, 1.000 Eq, 27.1 μmol) in MeOH (1 mL) and HCl (4N in dioxane) (49.4 mg, 339 μL, 4 molar, 50 Eq, 1.35 mmol) was stirred vigorously for 15 minutes at 23° C. The light amber solution was concentrated down and dried under high vacuum to yield crude (3S,4R)-4-((2-(piperidin-4-yl)-4,5-dihydrothiazolo[4,5-h]quinazolin-8-yl)amino)tetrahydro-2H-pyran-3-ol HCl (25.5 mg, 27 μmol, 100%, 45% Purity) as a thick, light amber oil, which was used immediately for the next step. LCMS RT=0.66 min., ESI MS (M+1) m/z=388.21.

### (3S,4R)-4-((2-(1-methylpiperidin-4-yl)-4,5-dihydrothiazolo[4,5-h]quinazolin-8-yl)amino)tetrahydro-2H-pyran-3-ol

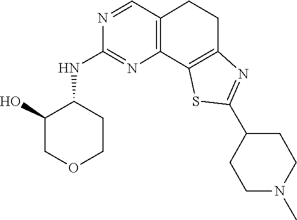 [SMILES: CN1CCC(c2nc3c(s2)-c2nc(N[C@@H]4CCOC[C@H]4O)ncc2CC3)CC1]

Step 8. To a stirred solution of (3S,4R)-4-((2-(piperidin-4-yl)-4,5-dihydrothiazolo[4,5-h]quinazolin-8-yl)amino)tetrahydro-2H-pyran-3-ol, HCl (11.5 mg, 1.000 Eq, 27.1 μmol) and triethylamine (13.7 mg, 18.9 μL, 5.000 Eq, 136 μmol) in DCM (1.5 mL) and MeOH (0.15 mL) was added paraformaldehyde (16.3 mg, 15.9 μL, 20.0 Eq, 542 mol). The mixture was stirred for 30 minutes before addition of sodium triacetoxyborohydride (28.7 mg, 5.00 Eq, 136 μmol). The reaction was stirred for 30 minutes after the addition of reducing agent. The reaction was quenched with aqueous 2N NaOH (~3 mL), stirred for 10 minutes, and then extracted with DCM (3×4 mL). The combined organic extracts were dried over sodium sulfate, filtered, concentrated down, and purified by silica gel chromatography with a DCM to 10:90:0.5 MeOH/DCM/NH₄OH gradient to yield (3S,4R)-4-((2-(1-methylpiperidin-4-yl)-4,5-dihydrothiazolo[4,5-h]quinazolin-8-yl)amino)tetrahydro-2H-pyran-3-ol (7.4 mg, 18 μmol, 65%, 95% Purity) as a white, foamy solid. LCMS RT=0.70 min., ESI MS (M+1) m/z=402.26. ¹H NMR (400 MHz, DMSO) δ 8.10 (s, 1H), 6.90 (d, J=7.6 Hz, 1H), 4.94 (d, J=5.1 Hz, 1H), 3.82-3.70 (m, 3H), 3.51-3.40 (m, 1H), 3.29-3.26 (m, 1H), 3.02 (dd, J=11.1, 9.7 Hz, 1H), 2.98-2.76 (m, 7H), 2.17 (s, 3H), 2.05-1.87 (m, 5H), 1.71 (qd, J=12.1, 3.7 Hz, 2H), 1.43 (qd, J=11.8, 4.5 Hz, 1H).

### Example 63

Compounds of the present disclosure were tested based on a CDK4/Cyclin D1 fluorescence-based microfluidic mobility shift assay (PerkinElmer).

The active wild type CDK4/Cyclin D1 complex was purchased from Eurofins (14-475). Compound stocks were prepared in DMSO and were serially diluted into 11 concentrations by 3-fold dilution. Into a 384-well plate (Greiner, 781201) 200 nL compounds were transferred to individual wells prior to adding 15 μL of 1.3× enzyme and ATP solution containing 0.13 nM CDK4/CyclinE and 2660 uM ATP in 1× reaction buffer containing 10 mM Hepes pH 7.5, 0.01% Brij-35, 10 mM MgCl₂, 1 mM EGTA, 0.05% BSA and 2 mM DTT. Compounds were incubated in the presence of CDK4/Cyclin D1 complex and ATP for 30 minutes at room temperature. Catalytic reaction was initiated with the addition of 5 μL of a 4× peptide solution containing 6 uM fluorescently label substrate peptide, FL Peptide 18 (aa sequence 5-FAM-QSPKKG, PerkinElmer, 760362) in 1× reaction buffer.

The final reaction components were 0.1 nM CDK4/Cyclin D1, 2000 uM ATP and 1.5 uM FL Peptide 18 and 1% DMSO. Reactions were incubated RT for 20 hr and terminated with 75 μL of a stopping solution containing 0.5 M EDTA. Samples were analyzed using a LabChip EZ reader (PerkinElmer).

Results of the CDK4 Biochemical Caliper Assay are presented in Table 1. Compounds having an IC50 less than or equal to 100 nM are represented as “A”; compounds having an IC50 greater than 100 nM but less than or equal to 250 nM are represented as “B”; compounds having an IC50 greater than 250 nM but less than or equal to 1 μM are represented as “C”; and compounds having an IC50 greater than 1 μM but less than or equal to 100 μM are represented as “D”.

### Example 64

Compounds described herein were also tested in a nanoBRET TE Intracellular Kinase Assay. Test compounds were prepared in a DMSO stock solution. 45 μL of stock solution was transferred to a 384 well plate (Greiner, 781201), and a 3-fold 11-point dilution was performed. 293-NB2 cells expressing target tagged with nanoLuciferase were diluted in cell growth media (DMEM+10% FBS+1% Pen/Strep media) and a 40 uL cell solution of 7.5E4 cells/mL was seeded in a separate 384 well cell culture plate and incubated overnight at at 37° C./5% CO₂/100% humidity. 2 μL of 20× Complete NanoBRET K-10 Tracer (Promega NanoBRET) was added to each well of the cell plate except control wells. 80 nL of each concentration of compound was transferred from the compound plate to a corresponding well in the cell culture plate by Echo550 liquid handler. The plates were incubated for 2 hrs at 37° C./5% CO₂/100% humidity. 20 μL of 3× Complete Nano-Glo Substrate & ECD NanoLuc Inhibitor (Promega NanoBRET) was added into each well. Chemiluminescence was then read on an EnVision reader (PerkinElmer).

Results of the Cell nanoBRET Assay are presented in Table 1. Compounds having an IC50 less than or equal to 100 nM are represented as “A”; compounds having an IC50 greater than 100 nM but less than or equal to 250 nM are represented as “B”; compounds having an IC50 greater than 250 nM but less than or equal to 1 μM are represented as “C”; and compounds having an IC50 greater than 1 μM but less than or equal to 100 μM are represented as “D”.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

LENGTHY TABLES
The patent application contains a lengthy table section. A copy of the table is available in electronic form from the USPTO web site (https://seqdata.uspto.gov/docdetail?docId=US20260193248A1). An electronic copy of the table will also be available from the USPTO upon request and payment of the fee set forth in 37 CFR 1.19(b)(3).

What is claimed is:

1. A compound of formula I′: 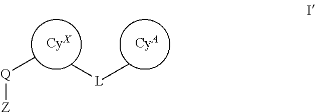
or a pharmaceutically acceptable salt thereof, wherein:
Q is L¹;
Z is H, halogen, or Cy^{B};
L is a covalent bond or a bivalent C₁₋₃ aliphatic group, wherein one carbon is optionally replaced by —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, —S(O)₂—, C₃₋₆ cycloalkylene, or 3-6 membered heterocycloalkylene having 1-2 heteroatoms independently selected from nitrogen, oxygen, and sulfur; wherein each of said C₃₋₆ cycloalkylene and 3-6 membered heterocycloalkylene is optionally substituted with one instance of R¹ or C₁₋₆ aliphatic;
Cy^{A} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{A} is substituted with m instances of R^{A};
Cy^{B} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{B} is substituted with n instances of R^{B};
Cy^{X} is phenyl, naphthyl, a 5-6 membered monocyclic heteroaryl ring having 1-4 heteroatoms independently selected from nitrogen, oxygen, phosphorus, and sulfur; an 8-12 membered bicyclic heteroaryl ring having 1-4 heteroatoms independently selected from nitrogen, oxygen, phosphorus, and sulfur; a 7-12 membered saturated, partially unsaturated, or aromatic bicyclic heterocyclic ring having 1-4 heteroatoms independently selected from nitrogen, oxygen, phosphorus, and sulfur; or a 10-15 membered saturated, partially unsaturated, or aromatic tricyclic heterocyclic ring having 1-6 heteroatoms independently selected from nitrogen, oxygen, phosphorus, and sulfur;
wherein Cy^{X} is substituted with u instances of L²-X⁰;
X⁰ is hydrogen, a halogen, oxo, CN, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{X};
Y is N or CH;
each instance of R^{A}, R^{B}, and R^{X} is independently R¹ or R², wherein R^{A} is substituted by q^{A} instances of R³, R^{B} is substituted by q^{B} instances of R³, and R^{X} is substituted with q^{X} instances of R³; or
two instances of R^{A}, two instances of R^{B}, two instances of R^{X}, two instances of X⁰, an instance of X⁰ and an instance of R^{A}, or an instance of R^{A} and an instance of R^{X} are taken together with their intervening atoms to form a 3-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R or —P(O)R²;
each instance of R² is independently C₁₋₇ aliphatic; —O—C₁₋₇ aliphatic; C₁₋₄ haloalkyl; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{X}, r, t, and u is independently 0, 1, 2, 3, or 4.

2. The compound of claim 1, wherein the compound is of formula I: 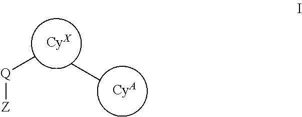
or a pharmaceutically acceptable salt thereof, wherein:
Q is L¹;
Z is H or Cy^{B};
Cy^{A} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur; wherein Cy^{A} is substituted with m instances of R^{A};
Cy^{B} is a phenyl; a 5-6 membered monocyclic heteroaryl ring having 1-4 heteroatoms independently selected from nitrogen, oxygen, phosphorus, and sulfur; an 8-10 membered bicyclic heteroaryl ring having 1-4 heteroatoms independently selected from nitrogen, oxygen, phosphorus, and sulfur; a 3-8 membered saturated or partially unsaturated monocyclic carbocyclic ring; a 3-8 membered saturated or partially unsaturated monocyclic heterocyclic ring having 1-2 heteroatoms independently selected from nitrogen, oxygen, phosphorus, and sulfur; a 7-12 membered saturated or partially unsaturated bicyclic heterocyclic ring having 1-4 heteroatoms independently selected from nitrogen, oxygen, phosphorus, and sulfur; a 10-14 membered saturated or partially unsaturated tricyclic heterocyclic ring having 1-6 heteroatoms independently selected from nitrogen, oxygen, phosphorus, and sulfur; or a 10-14 membered tricyclic heteroaryl ring having 1-6 heteroatoms independently selected from nitrogen, oxygen, and sulfur wherein Cy^{B} is substituted with n instances of R^{B};
Cy^{X} is 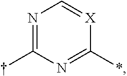
wherein [symbol] represents a bond to Q, and [symbol] represents a bond to Cy^{A};
X is N or CL²-X⁰;
X⁰ is hydrogen, a halogen, or a group selected from C₁₋₈ aliphatic, a saturated or partially unsaturated 3-14 membered monocyclic or bicyclic carbocyclic ring, phenyl, a 3-10 membered saturated or partially unsaturated monocyclic or bicyclic heterocyclic ring having 1-4 heteroatoms independently selected from nitrogen, oxygen, and sulfur, and a 5-14 membered monocyclic or bicyclic heteroaryl ring having 1-4 heteroatoms independently selected from nitrogen, oxygen, and sulfur; wherein X⁰ is substituted with p instances of R^{X};
Y is N or CH;
each instance of R^{A}, R^{B}, and R^{X} is independently R¹ or R², wherein R^{A} is substituted by q^{A} instances of R³, R^{B} is substituted by q^{B} instances of R³, and R^{X} is substituted with q^{X} instances of R³; or
two instances of R^{A}, two instances of R^{B}, two instances of R^{X}, or an instance of R^{A} and an instance of R^{X} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene having 1-2 heteroatoms independently selected from nitrogen, oxygen, and sulfur, 5-6 membered heteroarylene having 1-3 heteroatoms independently selected from nitrogen, oxygen, and sulfur, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; —O—C₁₋₇ aliphatic; C₁₋₄ haloalkyl; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{X}, r, and t is independently 0, 1, 2, 3, or 4.

3. The compound of claim 1 or 2, wherein Z is Cy^{B}.

4. The compound of claim 1 or 2, wherein Z is hydrogen.

5. The compound of any one of claims 1 to 4, wherein Q is a covalent bond, or a C₁₋₆ saturated or unsaturated, straight or branched hydrocarbon chain wherein one or two methylene units of the chain are optionally and independently replaced by —CH(R^{L})—, —C(R^{L})₂—, C₃₋₆ cycloalkylene, —NH—, —N(R^{L})—, or —O—.

6. The compound of any one of claims 1 to 5, Q is —NH—, 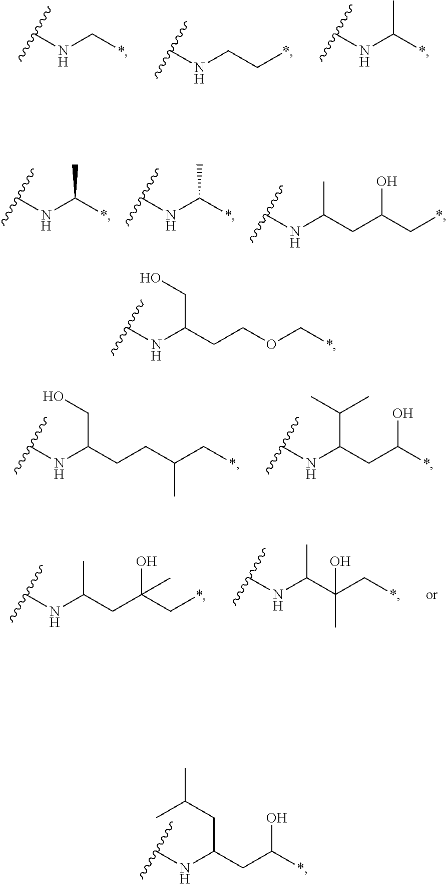
wherein 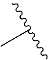
represents a covalent bond to Cy^{X} and [symbol] represents a covalent bond to Cy^{B} or Z.

7. The compound of claim 1, wherein the compound is of formula I-A: 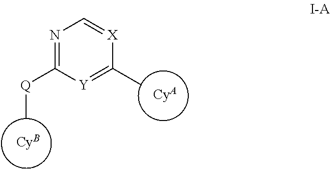
or a pharmaceutically acceptable salt thereof.

8. The compound of any one of claims 1-7, wherein Y is N.

9. The compound of any one of claims 1-7, wherein Y is CH.

10. The compound of any one of claims 1-9, wherein X is-CL²-X⁰.

11. The compound of any one of claims 1-10, wherein L² is a covalent bond.

12. The compound of claim 1, wherein the compound is of formula II, III, or IV: 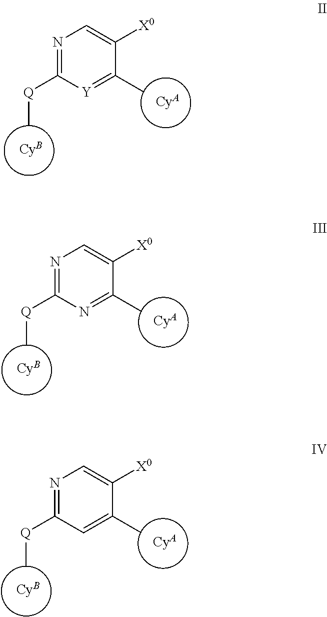
or a pharmaceutically acceptable salt thereof.

13. The compound of any one of claims 1-12, wherein Cy^{A} is an 8-10 membered bicyclic heteroaryl ring having 1-4 heteroatoms independently selected from nitrogen, oxygen, and sulfur.

14. The compound of any one of claims 1-12, wherein Cy^{A} is

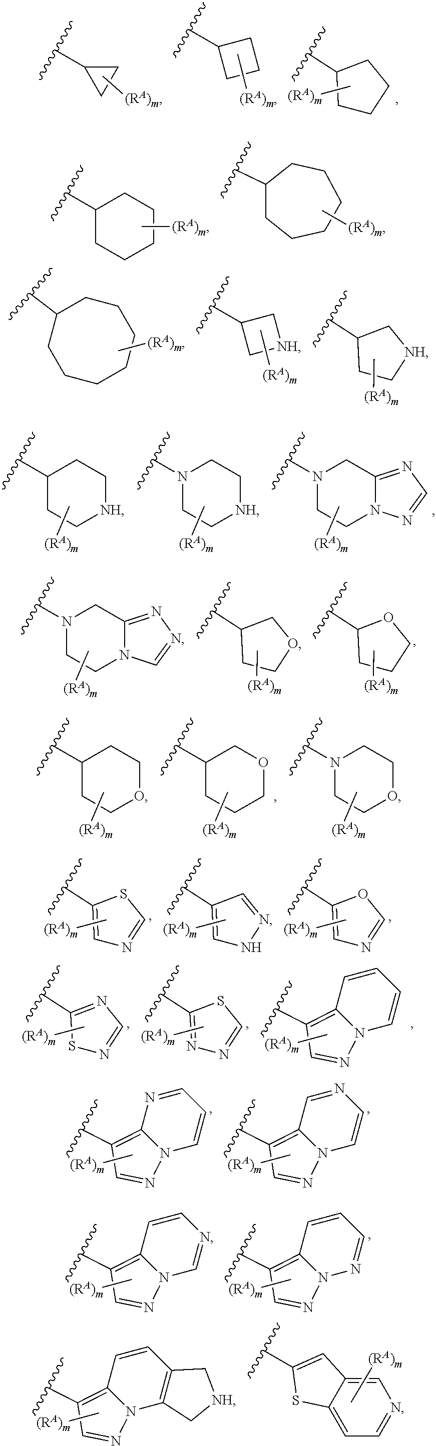

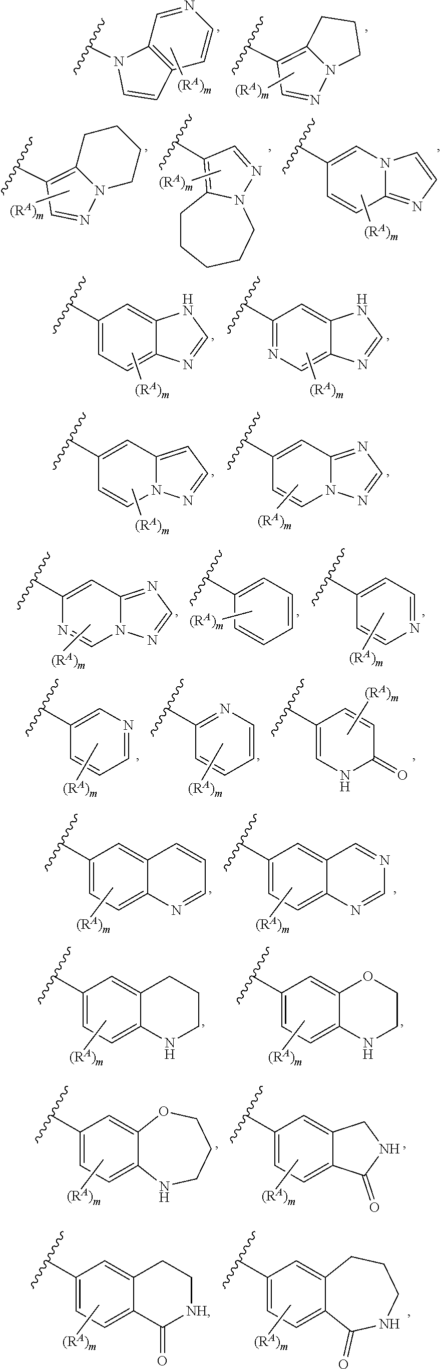

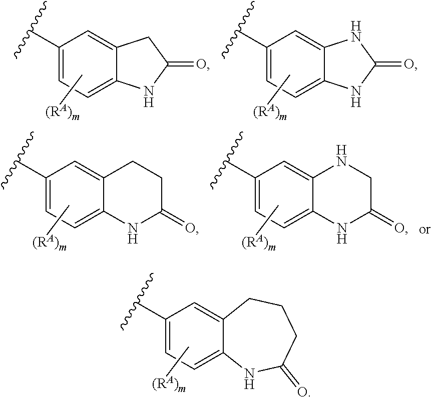

15. The compound of claim 1, wherein the compound is of formula II-A, II-B, II-C, II-D, II-E, II-X, II-Y, II-Z, II-AA, II-BB, II-CC, II-DD, II-EE, II-FF, II-GG, II-HH, II-II, II-JJ, II-KK, II-LL, II-MM, II-NN, II-OO, II-PP, II-QQ, II-RR, II-SS, II-TT, II-UU, or II-VV: 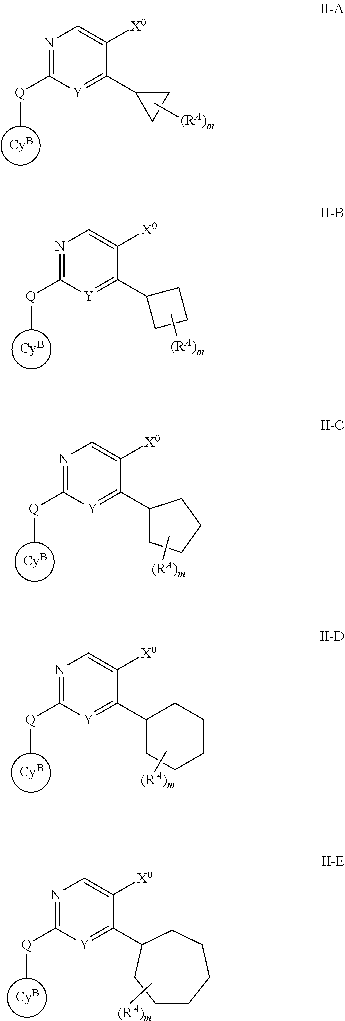 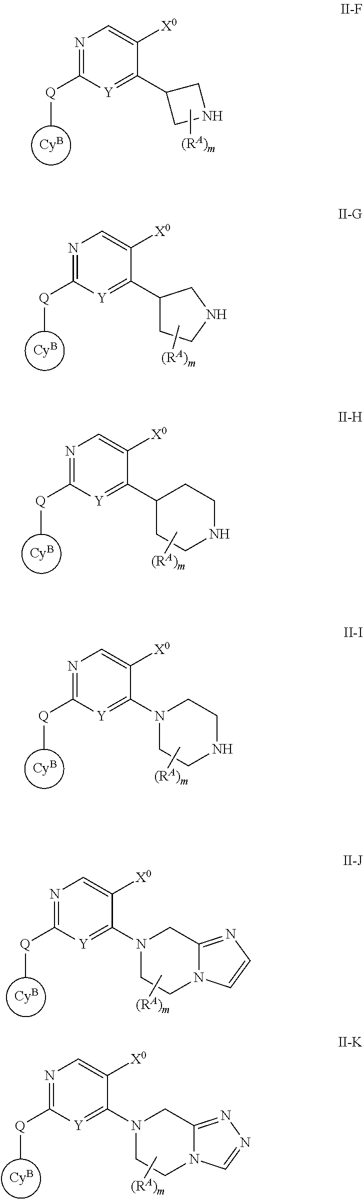 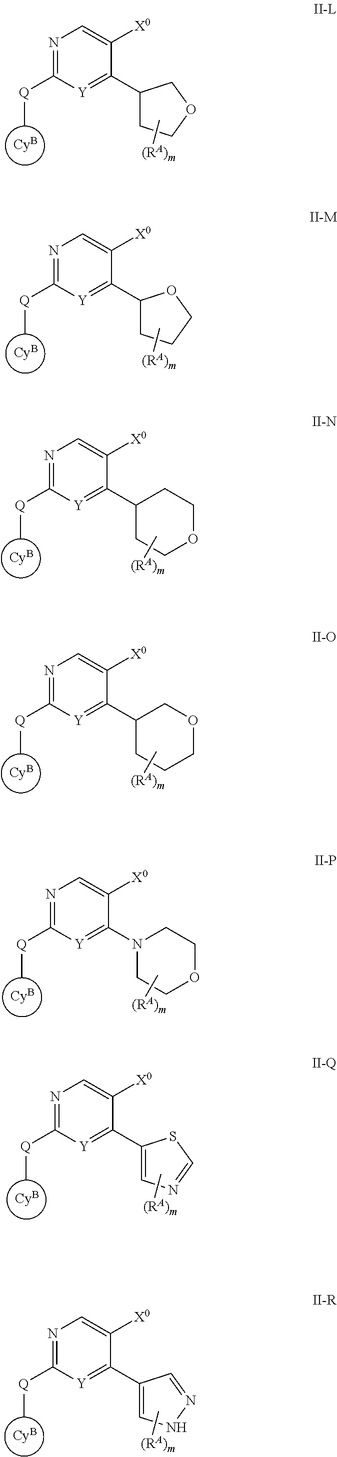 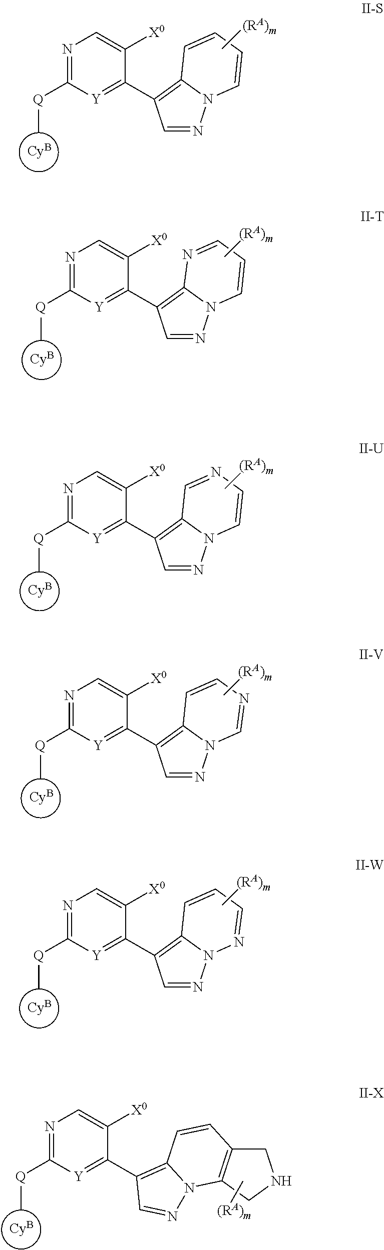 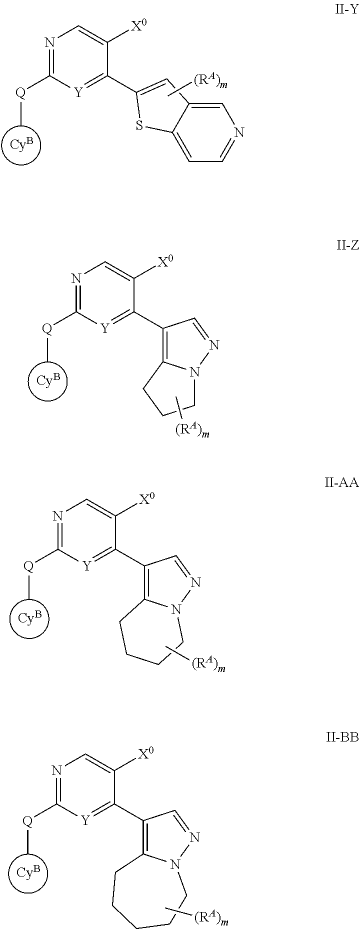 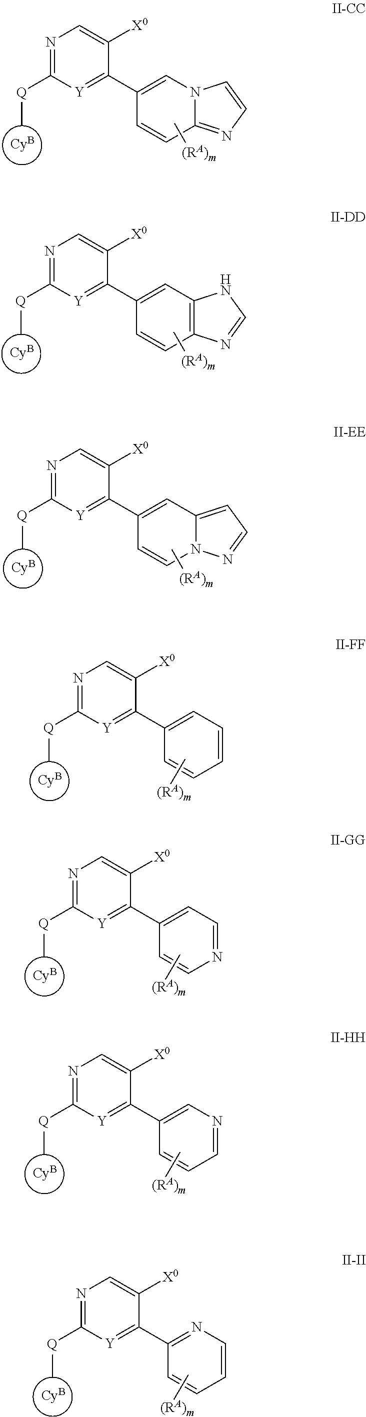 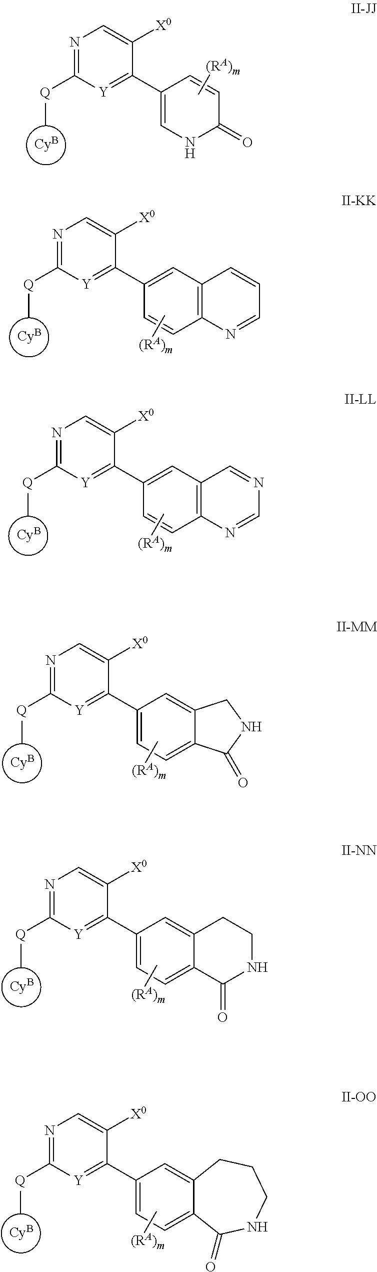 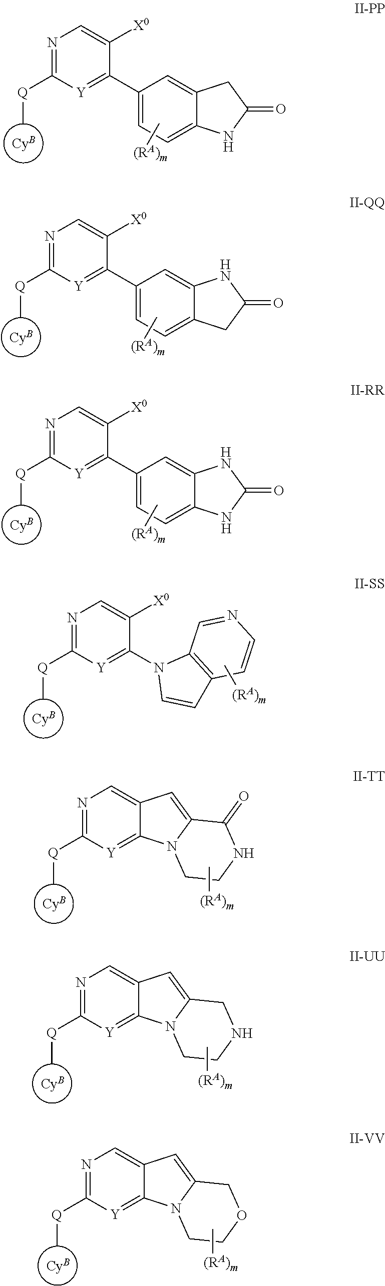
or a pharmaceutically acceptable salt thereof.

16. The compound of any one of claims 1-15, wherein Cy^{B} is a 3-8 membered saturated carbocyclic ring, wherein said ring is substituted with n instances of R^{B}.

17. The compound of any one of claims 1-15, wherein Cy^{B} is a 5-6 membered monocyclic heteroaryl ring having 1-4 heteroatoms independently selected from nitrogen, oxygen, and sulfur, wherein said ring is substituted with n instances of R^{B}.

18. The compound of claim 1, wherein the compound is of formula II-A1, II-B1, II-C1, II-D1, II-E1, II-F1, II-G1, II-H1, II-I1, II-J1, II-K1, II-L1, II-M1, II-N1, II-O1, II-P1, or II-Q1: 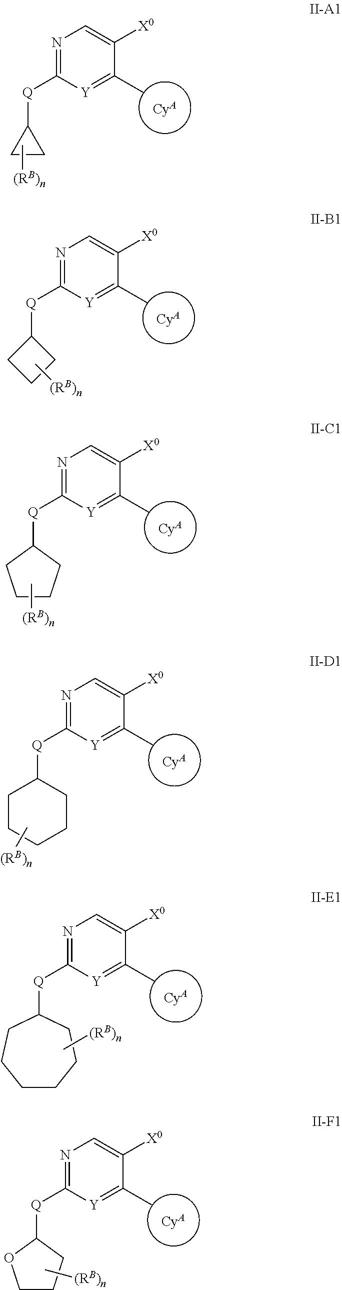 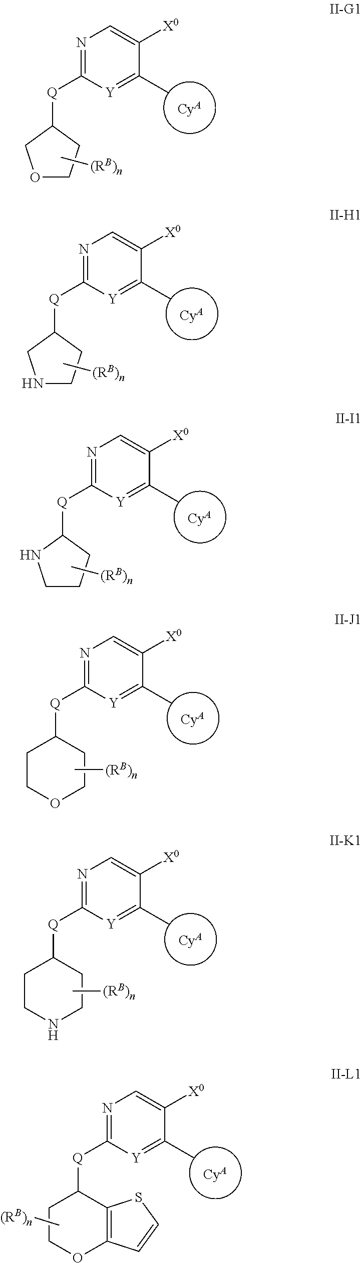 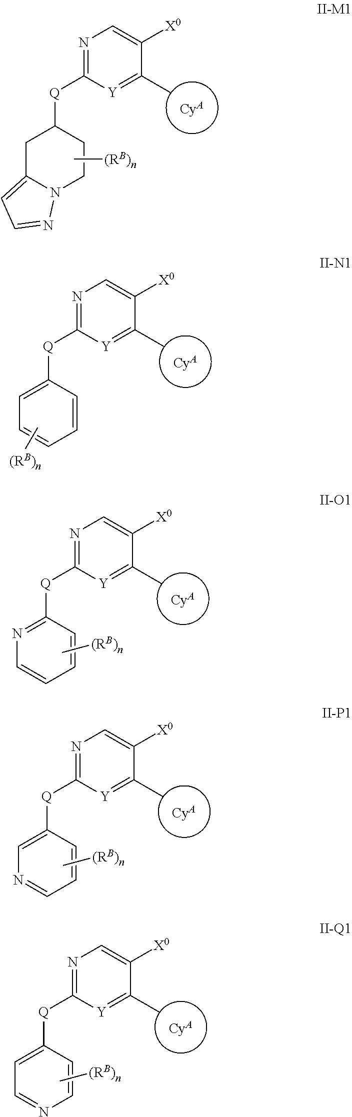
or a pharmaceutically acceptable salt thereof.

19. The compound of claim 1, wherein the compound is of formula II-A2, II-B2, II-C2, II-D2, II-E2, II-F2, II-G2, or II-H2: 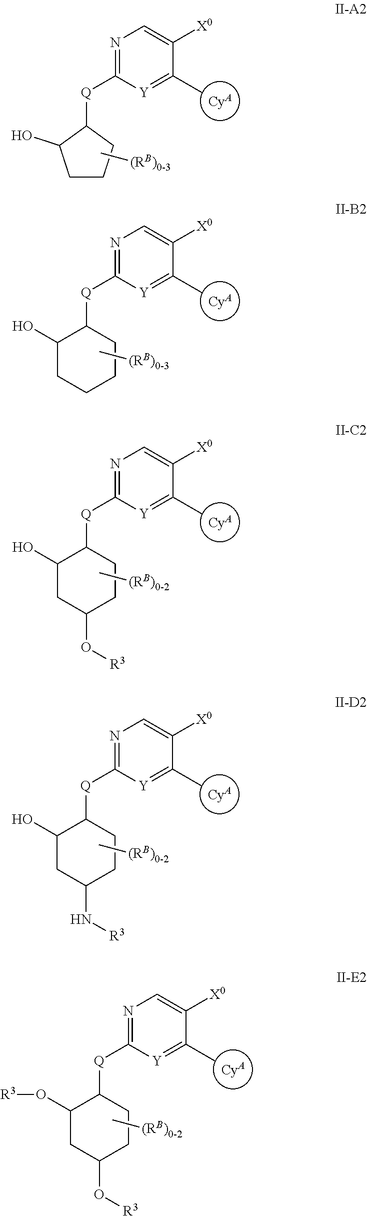 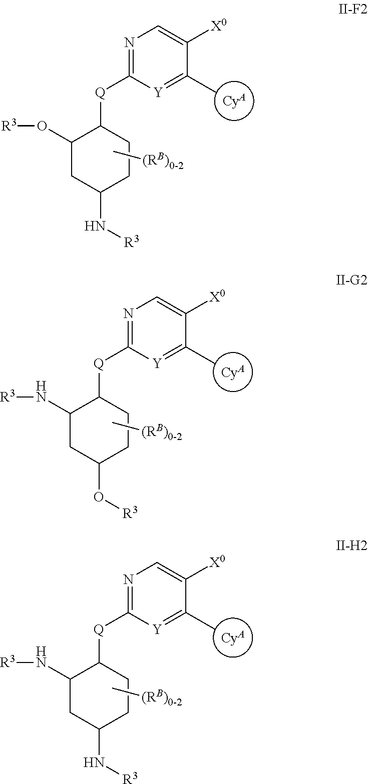
or a pharmaceutically acceptable salt thereof.

20. The compound of claim 1, wherein the compound is of formula V, VI, VII, VIII, IX, X, XI, XII, XIII, XIV, XV, XVI, XVII, XVIII, XIX, XX, XXI, or XXII: 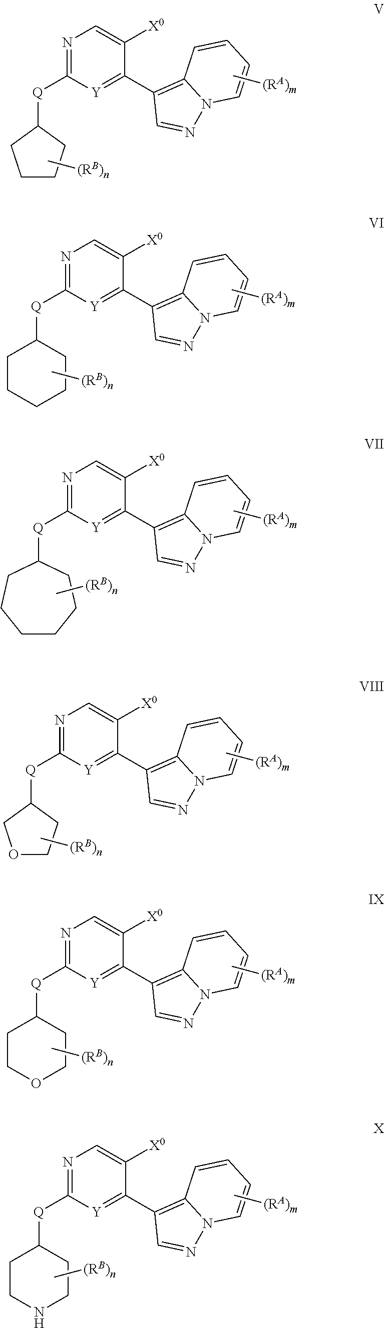 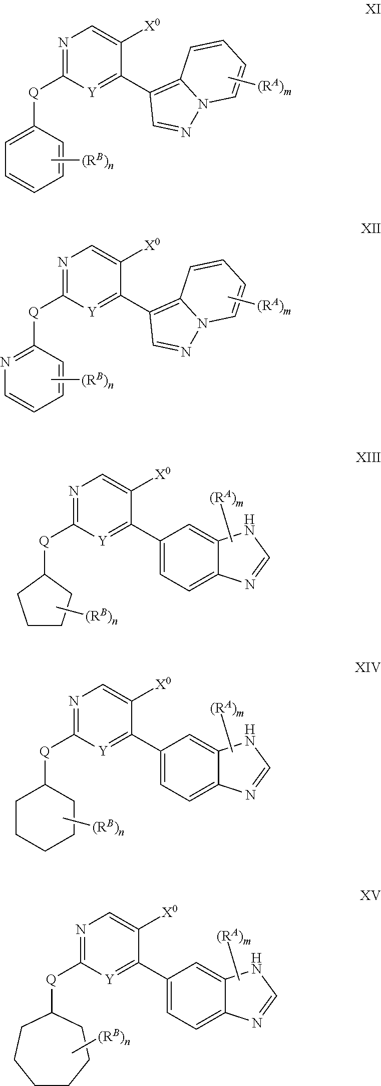 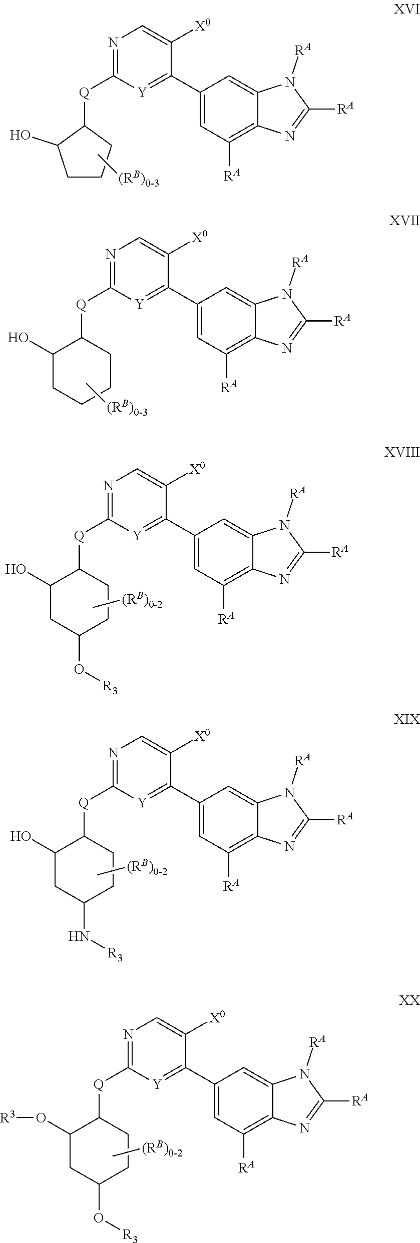 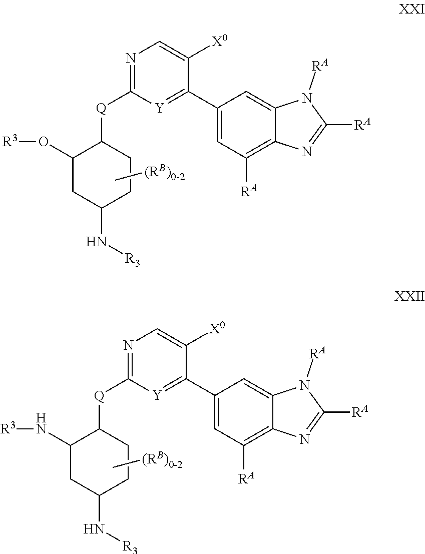
or a pharmaceutically acceptable salt thereof.

21. The compound of claim 1, wherein the compound is of formula XXIII, XXIV, XXV, XXVI, or XXVII: 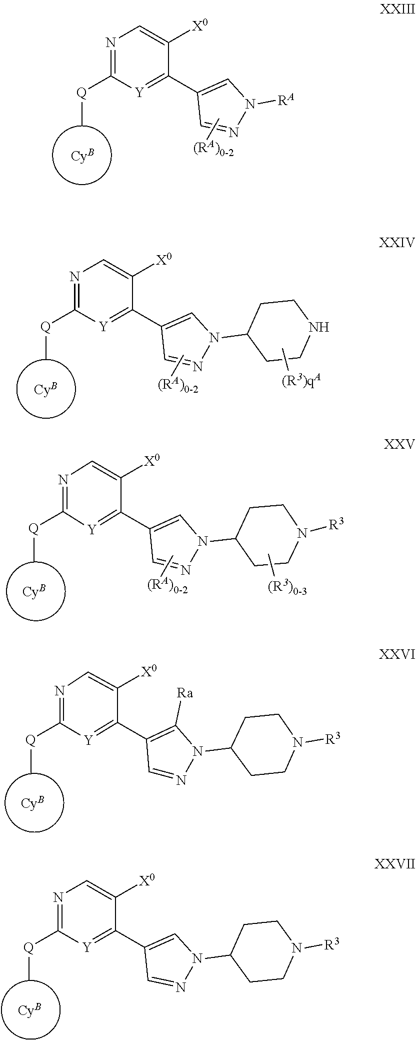
or a pharmaceutically acceptable salt thereof.

22. The compound of claim 1, wherein the compound is of formula XXVIII, XXIX, XXX, XXXI, XXXII, XXXIII, XXXIV, XXXV, XXXVI, XXXVII, XXXVIII, or XXXIX: 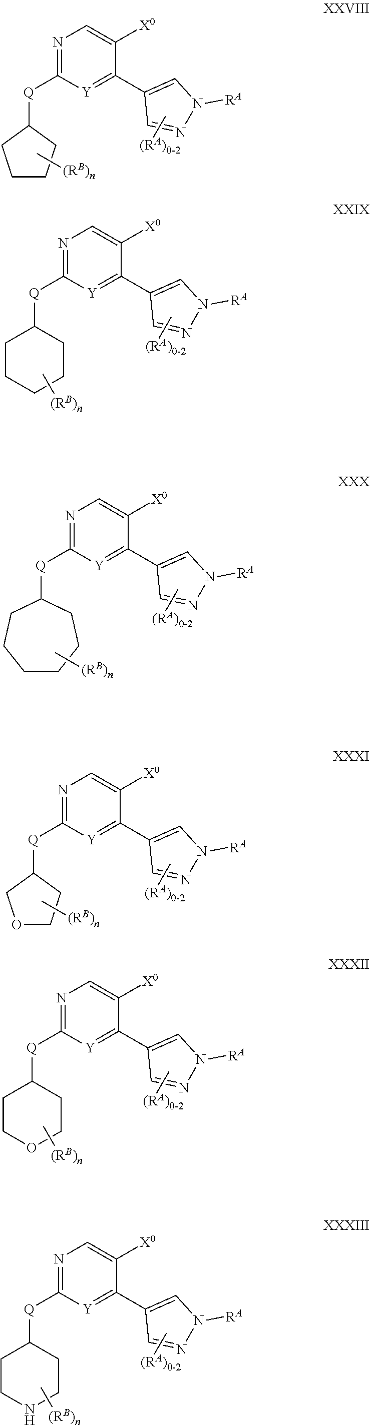 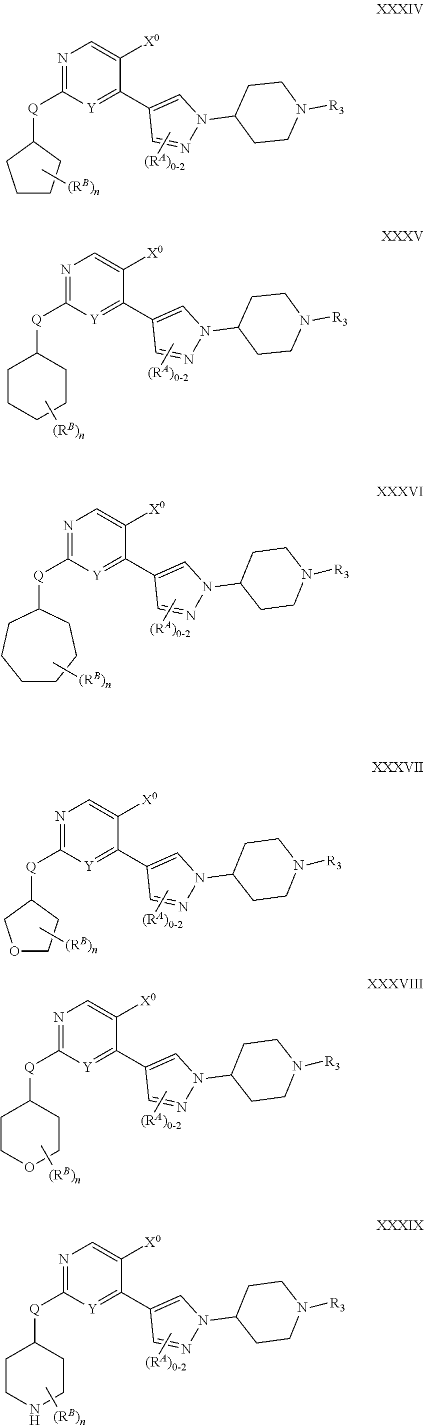
or a pharmaceutically acceptable salt thereof.

23. The compound of claim 1, wherein the compound is of formula XL, XLI, XLII, or XLIII: 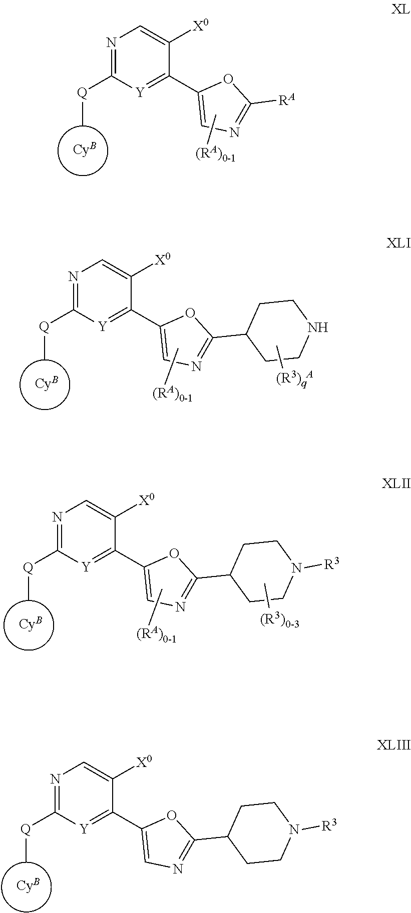
or a pharmaceutically acceptable salt thereof.

24. The compound of claim 1, wherein the compound is of formula XLIV, XLV, XLVI, XLVII, XLVIII, XLIX, L, LI, LII, LIII, LIV, or LV: 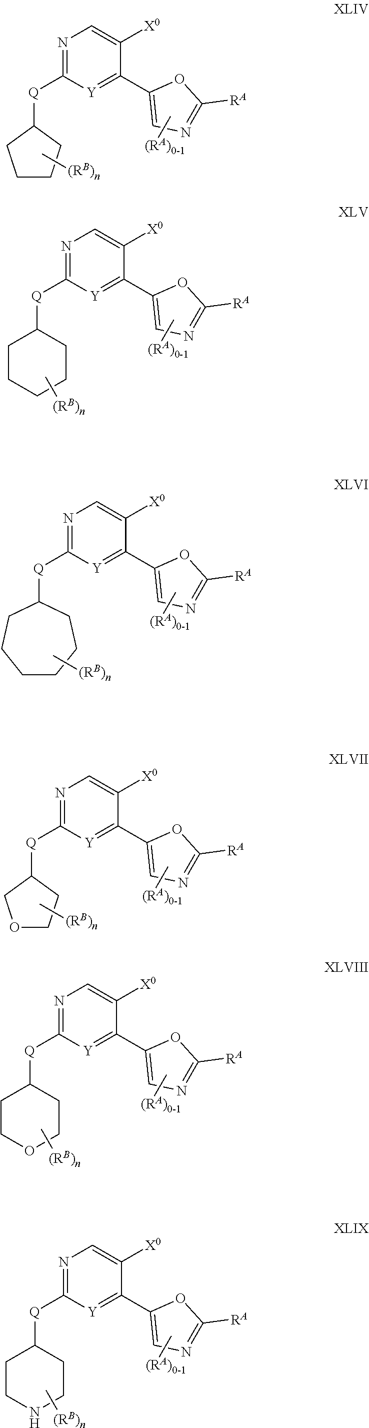 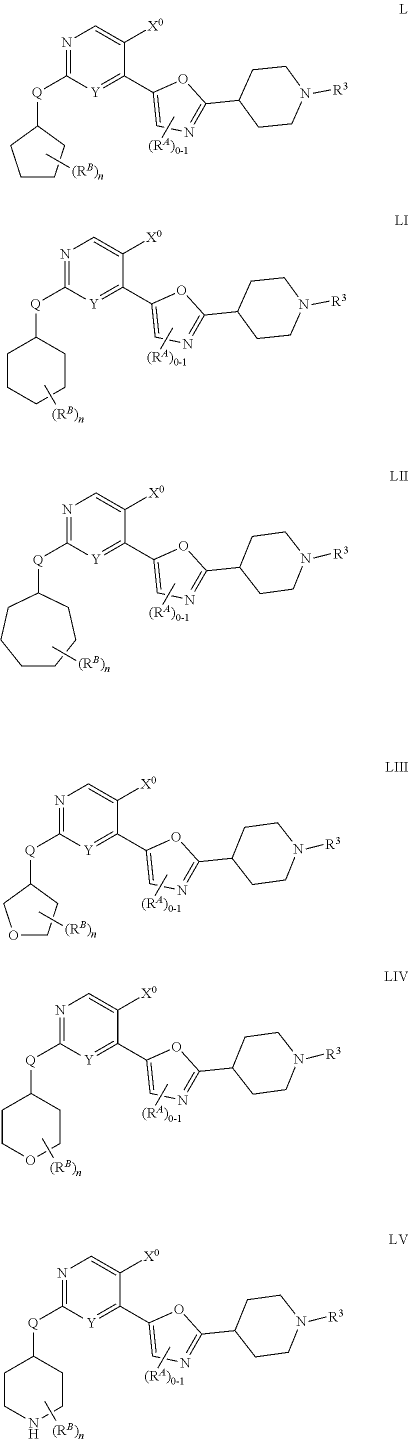
or a pharmaceutically acceptable salt thereof.

25. The compound of claim 1, wherein the compound is of formula LVI, LVII, LVIII, or LIX: 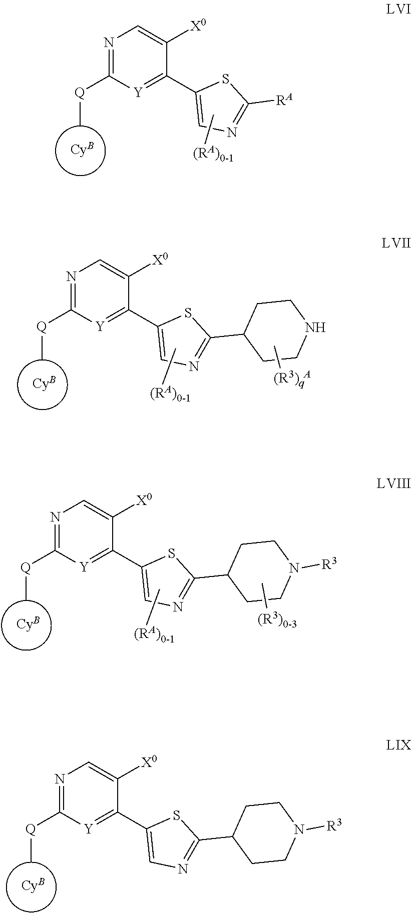
or a pharmaceutically acceptable salt thereof.

26. The compound of claim 1, wherein the compound is of formula LX, LXI, LXII, LXIII, LXIV, LXV, LXVI, LXVII, LXVIII, LXIX, LXX, or LXXI: 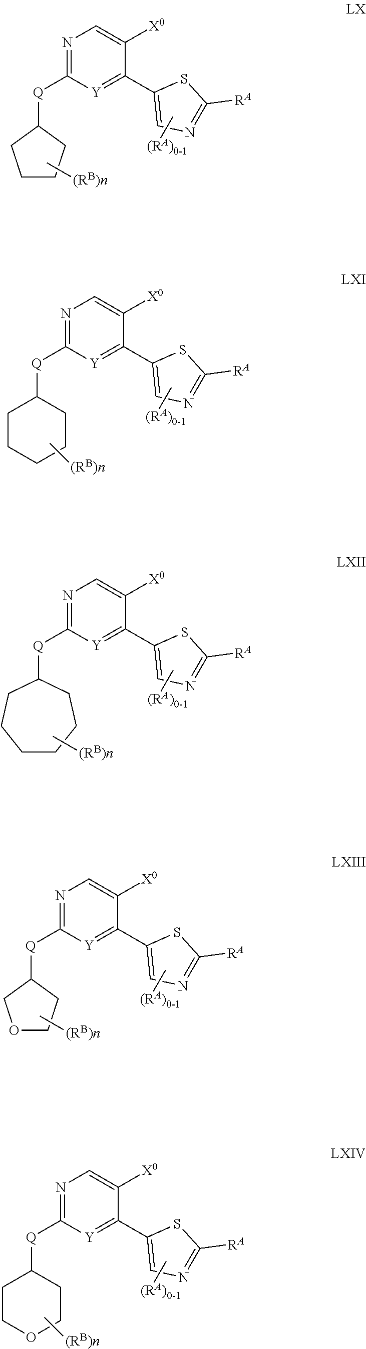 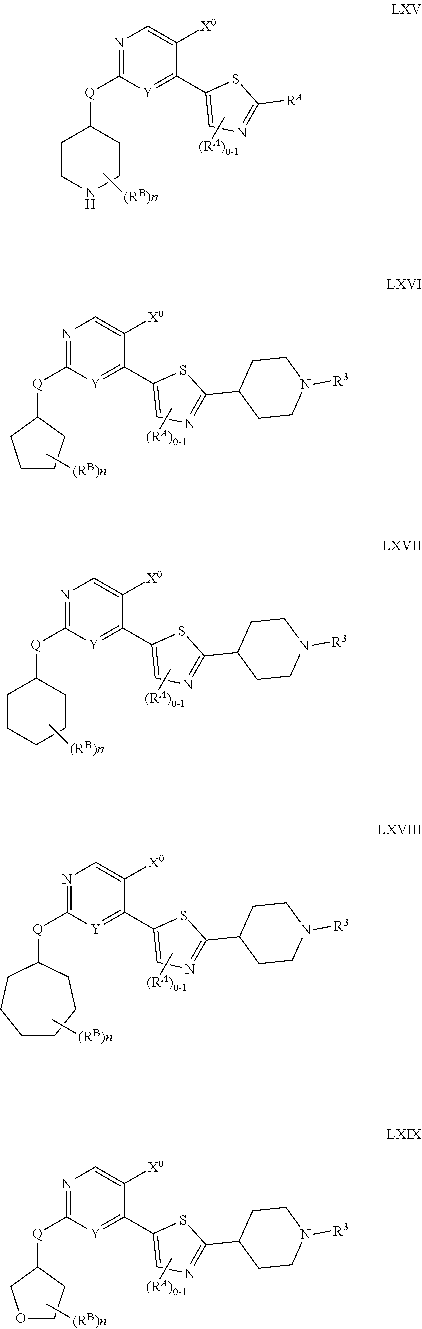 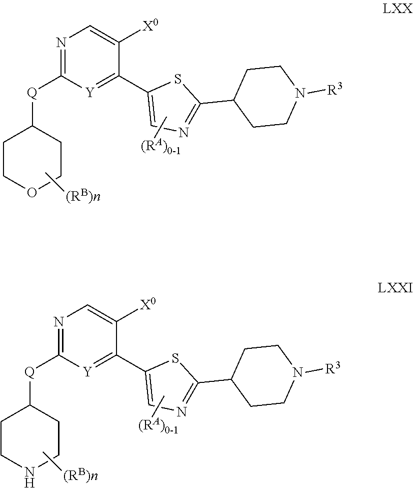
or a pharmaceutically acceptable salt thereof.

27. The compound of claim 1, wherein the compound is of formula LXXII, LXXIII, LXXIV, or LXXV: 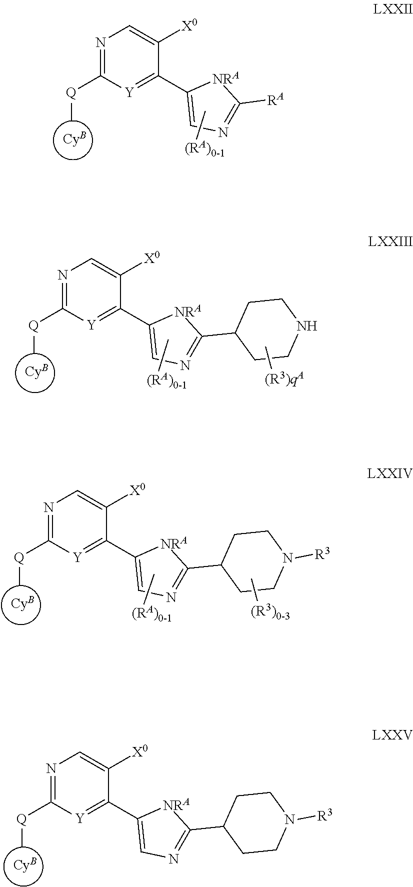
or a pharmaceutically acceptable salt thereof.

28. The compound of claim 1, wherein the compound is of formula LXXVI, LXXVII, LXXVIII, LXXIX, LXXX, LXXXI, LXXXII, LXXXIII, LXXXIV, LXXXV, LXXXVI, or LXXXVII: 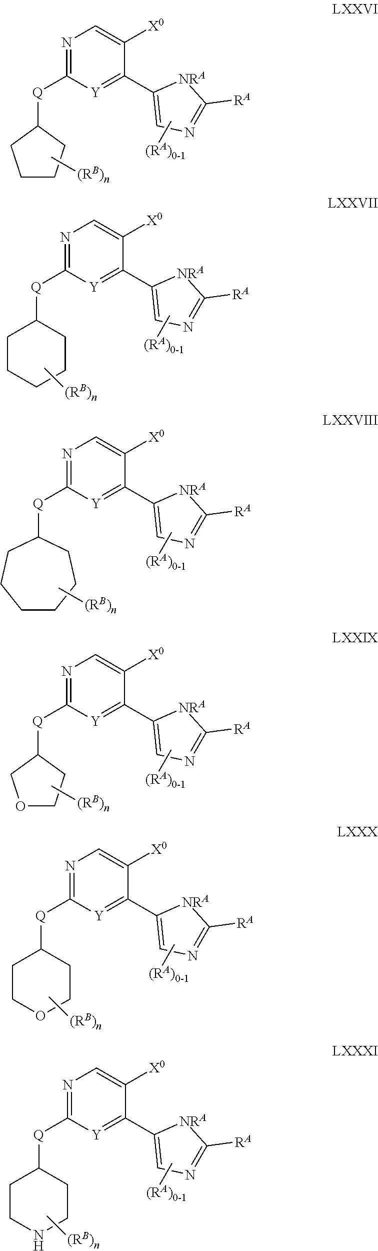 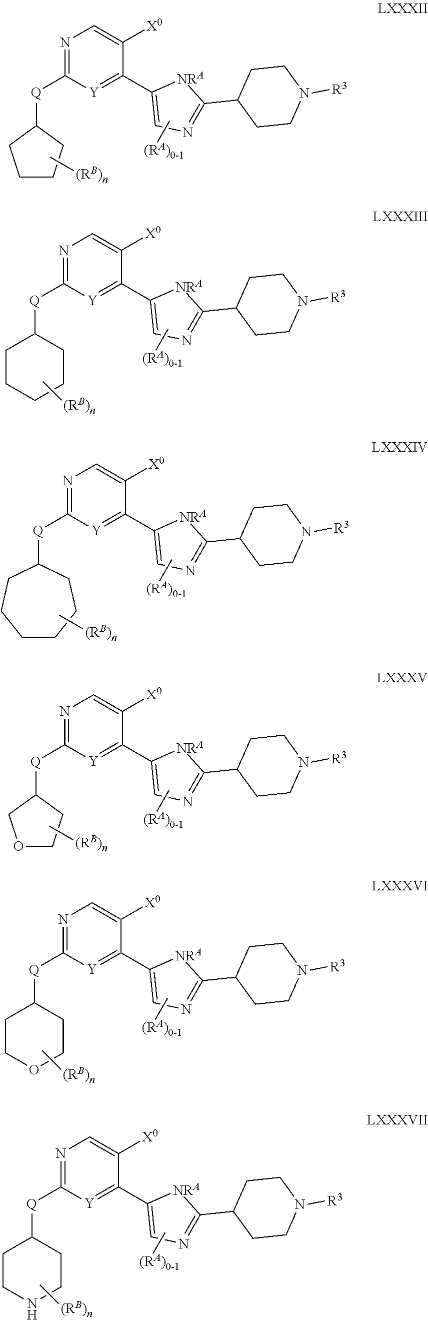
or a pharmaceutically acceptable salt thereof.

29. The compound of claim 1, wherein the compound is of formula LXXXVIII, LXXXIX, XC, or XCI: 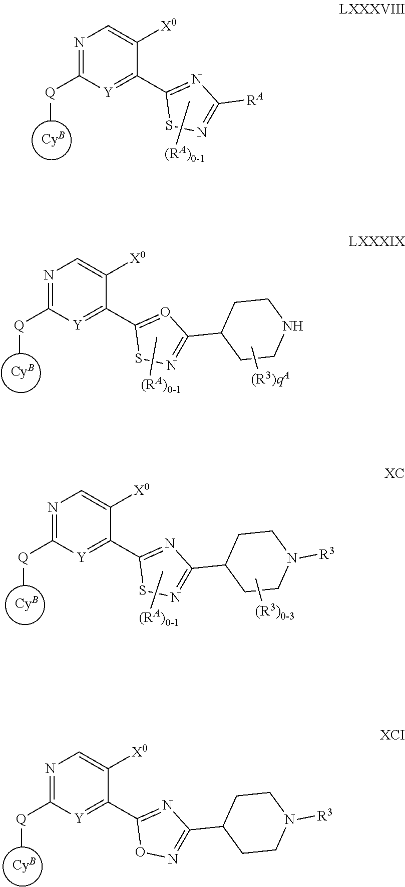
or a pharmaceutically acceptable salt thereof.

30. The compound of any one of claims 1-29, wherein X⁰ is halogen.

31. The compound of claim 30, wherein X⁰ is Cl.

32. The compound of claim 30, wherein X⁰ is F.

33. The compound of any one of claims 1-32, wherein Q is —NH—.

34. The compound of any one of claims 1-33, wherein at least one instance of R^{B} is oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.

35. The compound of any one of claims 1-33, wherein at least one instance of R^{B} is —OH.

36. The compound of any one of claims 1-35, wherein at least one instance of R^{A} is oxo, halogen, —CN, —NO₂, —OR, —SR, —NR₂, —S(O)₂R, —S(O)₂NR₂, —S(O)R, —S(O)NR₂, —SR(O)NR, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.

37. The compound of claim 36, wherein at least one instance of R^{A} is halogen.

38. The compound of any one of claims 1-35, wherein at least one instance of R^{A} is C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

39. The compound of claim 38, wherein at least one instance of R^{A} is C₁₋₇ aliphatic substituted with q^{A} instances of R³.

40. The compound of any one of claims 1-39, wherein at least one instance of R³ is oxo, halogen, —CN, —NO₂, —OR, —SR, —NR₂, —S(O)₂R, —S(O)₂NR₂, —S(O)R, —S(O)NR₂, —SR(O)NR, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.

41. The compound of any one of claims 1-40, wherein at least one instance of R³ is halogen.

42. The compound of any one of claims 1-40, wherein at least one instance of R^{is —S(O)}₂R, —S(O)₂NR₂, —C(O)OR, or —C(O)NR₂.

43. The compound of any one of claims 1-39, wherein at least one instance of R³ is an optionally substituted C₁₋₆ aliphatic.

44. The compound of claim 42, wherein at least one instance of R³ is methyl.

45. The compound of any one of claims 1-44, wherein the compound is selected from those depicted in Table 1, or a pharmaceutically acceptable salt thereof.

46. A pharmaceutical composition, comprising a compound of any one of claims 1-45, and a pharmaceutically acceptable carrier.

47. A method of inhibiting CDK4 signaling activity in a subject, comprising administering a therapeutically effective amount of a compound of any one of claims 1-45, or the pharmaceutical composition of claim 46, to a subject in need thereof.

48. A method of treating an CDK4-mediated disorder in a subject, comprising administering a therapeutically effective amount of a compound of any one of claims 1-45, or the pharmaceutical composition of claim 46, to a subject in need thereof.

49. A method of treating a disorder in a subject, wherein the disorder is ovarian cancer, endometrial cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, or pancreatic cancer, the method comprising administering to the subject a therapeutically effective amount of a compound of any one of claims 1-45, or the pharmaceutical composition of claim 46, to a subject in need thereof.

50. The method of claim 49, wherein the bladder cancer is urothelial carcinoma.

51. The method of claim 49, wherein the liver cancer is hepatocellular carcinoma.

52. The method of claim 49, wherein the lung cancer is lung squamous cell carcinoma or non-small cell lung cancer.

53. The method of claim 49, wherein the laryngeal cancer is laryngeal squamous cell carcinoma.

54. The method of claim 49, wherein the skin cancer is melanoma.
